(19) United States
(12) Patent Application Publication
(10) Pub. No.: US 2010/0184021 A1
(43) Pub. Date: Jul. 22, 2010

(54) NOVEL NUCLEOTIDE AND AMINO ACID SEQUENCES, AND METHODS OF USE THEREOF FOR DIAGNOSIS

(75) Inventors: Osnat Sella-Tavor, Kfar Kish (IL); Sarah Pollack, Tel-Aviv (IL); Lily Bazak, Givatayim (IL); Elena Tsypkin, Tel-Aviv (IL); Dan Sztybel, Tel-Aviv (IL); Shirley Sameah-Greenwald, Kfar Saba (IL)

Correspondence Address: FENNEMORE CRAIG, 3003 NORTH CENTRAL AVENUE, SUITE 2600, PHOENIX, AZ, 85012, US

(73) Assignee: COMPUGEN LTD.

(21) Appl. No.: 12/161,158

(22) PCT Filed: Jan. 16, 2007
(86) PCT No.: PCT/IL07/00056

§ 371 Date: Mar. 22, 2010

Related U.S. Application Data
(60) Provisional application No. 60/852,391, filed on Oct. 18, 2006.

(30) Foreign Application Priority Data
Jan. 16, 2006 (IL) 173173
Mar. 21, 2006 (IL) 174465

(51) Int. Cl. C12Q 1/68; C07H 21/04; C07K 14/00; C07K 16/00; C12N 15/63; C12N 5/10; C12P 21/02

(52) U.S. Cl. 435/6; 536/23.1; 530/324; 530/387.9; 435/320.1; 435/325; 435/69.1

(57) ABSTRACT

The present invention relates to diagnostic markers comprising novel splice variants of known proteins and polynucleotides encoding same, useful in the qualitative and/or quantitative detection of various diseases and/or pathological conditions in a subject, and to the use of known proteins and polynucleotides encoding same for diagnosis. Particularly, the invention relates to the diagnosis of a disease in a sample of body fluid or secretion obtained from the subject, and to the diagnosis of cancer.

### FIELD OF THE INVENTION

The present invention is related to novel nucleotide and protein sequences, and assays and methods of use thereof.

### BACKGROUND OF THE INVENTION

Diagnostic markers are important for early diagnosis of many diseases, as well as for predicting a response to treatment, monitoring treatment progress and determining prognosis of the disease.

Serum markers are examples of diagnostic markers, and are used for diagnosis of many different diseases. Typically, serum markers encompass secreted proteins and/or peptides; however, some serum markers may be released to the blood upon tissue lysis, for example from myocardial infarction (Troponin-I being a specific example). Serum markers can also be used as indicative risk factors of a disease (for example base-line levels of CRP, as a predictor of cardiovascular disease); to monitor disease activity and progression (for example, determination of CRP levels to monitor acute phase inflammatory response); and to predict and monitor drug response (for example, as shedded fragments of the protein Erb-B2).

Immunohistochemistry (IHC) is the study of the distribution of an antigen of choice in a sample based on specific antibody-antigen binding, typically performed on tissue slices. The antibody features a label which can be detected, for example as a stain which is detectable under a microscope. Preparation of the tissue slices for the assay involves fixation; IHC is therefore particularly suitable for antibody-antigen reactions that are not disturbed or destroyed by the process of fixing the tissue slices.

IHC permits determining the localization of the bound antibody-antigen, and hence mapping the presence of the antigen within the tissue and even within different compartments in the cell. Such mapping can provide useful diagnostic information, including:
1) The histological type of the tissue sample
2) The presence of specific cell types within the sample
3) Information regarding the physiological and/or pathological state of cells (e.g. which phase of the cell-cycle they are in)
4) The presence of disease related changes within the sample
5) Differentiation between specific disease subtypes where it is already known that the tissue is diseased (for example, the differentiation between different tumor types when it is already known the sample was taken from cancerous tissue).

IHC information is valuable for more than diagnosis. It can also be used to determine prognosis and progression of a therapy treatment (for example, as in the case of HER-2 in breast cancer) as well as to monitor the disease state.

IHC protein markers could be from any cellular location. Most often these markers are membrane proteins but secreted proteins or intracellular proteins (including intranuclear) can also be used as an IHC marker.

Although widely used as diagnostic tool, the IHC technique has at least two major disadvantages. It is performed on tissue samples and therefore a tissue sample has to be collected from the patient, which most often requires invasive procedures like biopsy associated with pain, discomfort, hospitalization and risk of infection. In addition, the interpretation of the result is observer dependent and therefore subjective. There is no measured value but rather only an estimation (on a scale of 1-4) of how prevalent the antigen is on the target.

Thus, there is a recognized need for, and it would be highly advantageous to have, an alternative diagnostic tool for diagnosing and monitoring diseases.

### SUMMARY OF THE INVENTION

The present invention provides novel nucleic acid and amino acid sequences, which can be used as diagnostic markers.

According to one aspect, the present invention provides a number of novel variants of known proteins which are found in serum and can be used as diagnostic markers. The:present invention overcomes the many deficiencies of the background art with regard to the need to obtain tissue samples and subjective interpretations of results. In certain embodiments of the present invention, tissue specific markers are identifiable in serum or plasma. Thus, according to the teachings of the present invention, a simple blood test can provide qualitative and/or quantitative indication of various diseases and/or pathological conditions, according to the expression of certain marker(s).

According to another aspect, the present invention discloses the novel use of known proteins as diagnostic markers. In some embodiments, the markers disclosed can also be used for in-vivo imaging applications.

It is disclosed in the present invention for the first time that the protein variants of the invention are useful as diagnostic markers for various diseases and/or pathological conditions as described in greater detail below. The variants themselves are described by “cluster” or by gene, as these variants are splice variants of known proteins. Therefore, as used in the present invention, the term “marker-detectable disease” refers to a disease that may be detected by a particular marker, with regard to the description of the disease provided herein below. The markers of the present invention, alone or in combination, show a high degree of differential diagnosis between disease and non-disease states.

The present invention further relates to diagnostic assays for detecting a disease, particularly in a sample taken from a subject (patient), preferably a blood sample or a body secretion sample. According to certain embodiments, the diagnostic assays disclosed in the present invention are NAT (nucleic acid amplification technology)-based assays, including, for example, PCR or variations thereof, e.g. real-time PCR. According to other embodiments, the assays encompass nucleic acid hybridization assays. The diagnostic assays can be qualitative or quantitative.

According to certain embodiments, the present invention provides a diagnostic marker comprising a novel splice variant of a known protein or a polynucleotide encoding same, wherein the protein is selected from the group consisting of Delta-like Protein 3 Precursor (DLL3), Complement Factor B Precursor, Serine/Threonine-Protein Kinase TNNI3K, Cardiomyopathy Associated 4 (CMYA4) and Myosin Regulatory Light Chain 2, Atrial Isoform. According to certain embodiments, the diagnostic marker is found in a body fluid or secretion.

According to one embodiment, the novel splice variant is an isolated polynucleotide comprising a nucleic acid having a nucleic acid sequence as set forth in any one of SEQ. ID NOs:37-39, 78-88, 156-160, 162-164, 167-170, 240-241, 276-281, or a sequence homologous thereto. According to one embodiment, the isolated polynucleotide is at least 85% homologous to any one of SEQ. ID NOs: 37-39, 78-88, 156-160, 162-164, 167-170, 240-241, 276-281.

According to another embodiment, the novel splice variant is an isolated polynucleotide comprising a nucleic acid having a nucleic acid sequence as set forth in any one of SEQ. ID NOs: 40-53, 89-130, 171-208, 242-266, 282-301, Or a sequence homologous thereto. According to one embodiment, the isolated polynucleotide is at least 85% homologous to any one of SEQ. ID NOs: 40-53, 89-130, 171-208, 242-266, 282-301.

According to certain embodiments, the present invention also encompasses isolated polynucleotides having a sequence complementary to any one of the nucleic acid sequences listed herein. According to other embodiments, this invention provides an oligonucleotide of at least about 12 nucleotides, specifically hybridizable with the polynucleotides of this invention. The present invention further provides vectors, cells, liposomes and compositions comprising the isolated polynucleotides of this invention.

According to yet another embodiment, the novel splice variant is an isolated protein or polypeptide having an amino acid sequence as set forth in any one of SEQ. ID NOs: 57-58, 135, 137, 138, 140-142, 212-220, 222-229, 268, 269, 303-308, or a sequence homologous thereto. According to one embodiment, the isolated protein or polypeptide is at least 85% homologous to any one of SEQ. ID NOs: 57-58, 135, 137, 138, 140-142, 212-220, 222-229, 268, 269, 303-308.

According to some embodiments, the sample taken from a subject (patient) to perform the diagnostic assay according to the present invention is selected from the group consisting of a body fluid or secretion including but not limited to blood, serum, urine, plasma, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, cyst fluid, secretions of the breast ductal system (and/or lavage thereof), broncho alveolar lavage, lavage of the reproductive system and lavage of any other part of the body or system in the body; samples of any organ including isolated cell(s) or tissue(s), wherein the cell or tissue can be obtained from an organ selected from, but not limited to lung, colon, ovarian and/or breast tissue; stool or a tissue sample, or any combination thereof. In some embodiments, the term encompasses samples of in vivo cell culture constituents. Prior to be subjected to the diagnostic assay, the sample can optionally be diluted with a suitable eluant.

The term “homology”, as used herein, refers to a degree of sequence similarity in terms of shared amino acid or nucleotide sequences. There may be partial homology or complete homology (i.e., identity). For amino acid sequence homology amino acid similarity matrices may be used as are known in different bioinformatics programs (e.g. BLAST, Smith Waterman). Different results may be obtained when performing a particular search with a different matrix. Homologous peptide or polypeptides are characterized by one or more amino acid substitutions, insertions or deletions, such as, but not limited to, conservative substitutions, provided that these changes do not affect the biological activity of the peptide or polypeptide as described herein.

Degrees of homology for nucleotide sequences are based upon identity matches with penalties made for gaps or insertions required to optimize the alignment, as is well known in the art (e.g. Altschul S. F. et al., 1990, J Mol Biol 215(3):403-10; Altschul S. F. et al., 1997, Nucleic Acids Res.

25:3389-3402). The degree of sequence homology is presented in terms of percentage, e.g. “70% homology”. As used herein, the term “at least” with regard to a certain degree of homology encompasses any degree of homology from the specified percentage up to 100%.

The terms “correspond” or “corresponding to” or “correspondence with” are used herein to indicate identity between two corresponding amino acid or nucleic acid sequences.

In some embodiments, the proteins or polypeptides of this invention comprise chimeric protein or polypeptides.

As used herein, the terms “chimeric protein or polypeptide”, or “chimeric polynucleotide” or “chimera” refers to an assembly or a string of amino acids in a particular sequence, or nucleotides encoding the same, respectively, which does not correspond in their entirety to the sequence of the known (wild type) polypeptide or protein, or the nucleic acid encoding same.

In some embodiments, the variants of this invention are derived from two exons, or an exon and an intron of a known protein, or fragments thereof, or segments having sequences with the indicated homology.

According to certain embodiments, the present invention now discloses a novel cluster designated herein N43992, comprising novel amino acid and nucleic acid sequences that are variants of the known Delta-like protein 3 precursor (DLL3, SEQ. ID NO: 54, SwissProt accession identifier DLL3_HUMAN), known also according to the synonym Drosophila Delta homolog 3. The novel variant polynucleotides and polypeptides described by the present invention are useful as diagnostic markers, preferably as serum markers.

Surprisingly, the present invention now shows that the wild type DLL3 as well as its variants are overexpressed in cancerous tissues, particularly in cancerous lung tissues, and thus can be used for the diagnosis, prognosis, treatment selection, and treatment monitoring and/or assessment of cancers, particularly lung cancer, as is described in a greater detail below. According to certain embodiments of the present invention, the wild type DLL3 polynucleotides and polypeptides are useful as diagnostic markers, preferably as IHC markers and for in-vivo imaging.

According to one embodiment, the present invention provides an isolated polypeptide comprising an edge portion of N43992_P13 (SEQ. ID NO: 57), wherein the edge portion comprises an amino acid sequence being at least 70%, optionally at least about 80%, preferably at least about 85%, more preferably at least about 90% and most preferably at least about 95% homologous to the amino acid sequence APLPPLLQSLPEAWALRGGRRVPVRPGRGAECARTGLHRAARSARA (SEQ. ID NO: 326), corresponding to amino acids 44-89 of N43992_P13 (SEQ. ID NO: 57).

According to one embodiment, the isolated polypeptide is a chimeric polypeptides comprising a first amino acid sequence being at least about 90% homologous to the amino acid sequence MVSPRMSGLLSQTVILALIELPQTRPAGVFELQIHSFGPGPGP, corresponding to amino acids 1-43 of DLL3_HUMAN (SEQ. ID NO: 54), also corresponding to amino acids 1-43 of N43992_P13 (SEQ. ID NO: 57), and a second amino acid sequence being at least about 70%, optionally at least about 80%, preferably at least about 85%, more preferably at least about 90% and most preferably at least about 95% homologous to the amino acid sequence APLPPLLQSLPEAWALRGGRRVPVRPGRGAECARTGLHRAARSARA (SEQ. ID NO: 326), corresponding to amino acids 44-89 of N43992_P13 (SEQ. ID NO: 57), wherein the first amino acid sequence and the second amino acid sequence are contiguous and in a sequential order.

According to another embodiment, the isolated polypeptide comprises an amino acid sequence as set forth in SEQ. ID NO:57 (N43992_P13).

According to another embodiment, the present invention provides an isolated polypeptide comprising an edge portion of N43992_P14 (SEQ. ID NO: 58), wherein the edge portion comprises an amino acid sequence being at least 70%, optionally at least about 80%, preferably at least about 85%, more preferably at least about 90% and most preferably at least about 95% homologous to the amino acid sequence VRARHGPLASSSCRSTLSGRVQALGPRGPPAAPGSPAASSSESA (SEQ. ID NO: 327), corresponding to amino acids 24-67 of N43992_P14 (SEQ. ID NO: 58).

According to one embodiment, the isolated polypeptides is a chimeric polypeptide comprising a first amino acid sequence being at least about 90% homologous to the amino acid sequence MVSPRMSGLLSQTVELALIFLPQ corresponding to amino acids 1-23 of DLL3_HUMAN (SEQ. ID NO: 54), also corresponding to amino acids 1-23 of N43992_P14 (SEQ. ID NO: 58), and a second amino acid sequence being at least about 70%, optionally at least about 80%, ₚreferably at least about 85%, more preferably at least about 90% and most preferably at least about 95% homologous to a polypeptide having the sequence VRARHGPLASSSCRSTLSGRVQALGPRGPPAAPGSPAASSSESA (SEQ. ID NO: 327) corresponding to amino acids 24-67 of N43992_P14 (SEQ. ID NO: 58), wherein the first amino acid sequence and the second amino acid sequence are contiguous and in a sequential order.

According to another embodiment, the isolated polypeptide comprises an amino acid sequence as set forth in SEQ. ID NO:58 (N43992_P14).

According to a further embodiment the present invention provides an isolated polypeptide comprising an edge portion of N43992_P16 (SEQ. ID NO: 59), wherein the edge portion comprises an amino acid sequence being at least 70%, optionally at least about 80%, preferably at least about 85%, more preferably at least about 90% and most preferably at least about 95% homologous to the amino acid sequence EAWRPERRGMGWGSWMAQTVQGWNPGFDSSNPRAWGPDLPPASL (SEQ. ID NO: 328), corresponding to amino acids 366-409 of N43992_P16 (SEQ. ID NO: 59).

According to one embodiment, the isolated polypeptide is a chimeric polypeptides comprising a first amino acid sequence being at least about 90% homologous to the amino acid sequence MVSPRMSGLLSQTVILALIFLPQTRPAGVFELQMISFGPGPGPGAPRSPCSARLPCRLFFRVCLK PGLSEEAAESPCALGAALSARGPVYTEQPGAPAPDLPLPDGLLQVPFRDAWPGTFSFBETWRE ELGDQIGGPAWSLLARVAGRRRLAAGGPWARDIQRAGAWELRFSYRARCEPPAVGTACTRL CRPRSAPSRCGPGLRPCAPLEDECEAPLVCRAGCSPEHGFCEQPGECRCLEGWTGPLCTVPVS TSSCLSPRGPSSATTGCLVPGPGPCDGNPCANGGSCSETPRSFECTCPRGFYGLRCEVSGVTCA DGPCFNGGLCVGGADPDSAYICHCPPGFQGSNCEKRVDRCSLQPCRNG corresponding to amino acids 1-365 of DLL3_HUMAN (SEQ. ID NO: 54), also corresponding to amino acids 1-365 of N43992_P16 (SEQ. ID NO: 59), and a second amino acid sequence being at least about 70%, optionally at least about 80%, preferably at least about 85%, more preferably at least about 90% and most preferably at least about 95% homologous to the amino acid sequence EAWRPERRGMGWGSWMAQTVQGWNPGFDSSNPRAWGPDLPPASL (SEQ. ID NO: 328) corresponding to amino acids 366-409 of N43992_P16 (SEQ. ID NO: 59), wherein said first amino acid sequence and second amino acid sequence are contiguous and in a sequential order.

According to another embodiment, the isolated chimeric polypeptides comprises a first amino acid sequence being at least about 90% homologous to the amino acid sequence MVSPRMSGLLSQTVILALIFLPQTRPAGVFELQIHSFGPGPGPGAPRSPCSARLPCRLFFRVCLK PGLSEEAAESPCALGAALSARGPVYTEQPGAPAPDLPLPDGLLQVPFRDAWPGTFSFIIETWRE ELGDQIGGPAWSLLARVAGRRRLAAGGPWARDIQRAGAWELR corresponding to amino acids 35. 1-171 of Q8NBS4_HUMAN (SEQ. ID NO: 55), also corresponding to amino acids 1-171 of N43992_P16 (SEQ. ID NO: 59), a first bridging amino acid F corresponding to amino acid 172 of N43992_P16 (SEQ. ID NO: 59), a second amino acid sequence being at least about 90% homologous to the amino acid sequence SYRARCEPPAVGTACTRLCRPRSAPSRCGPGLRPCAPLEDECEAP corresponding to amino acids 173-217 of Q8NBS4_HUMAN (SEQ. ID NO: 55), also corresponding to amino acids 173-217 of N43992_P16 (SEQ. ID NO: 59), a second bridging amino acid L corresponding to amino acid 218 of N43992_P16 (SEQ. ID NO: 59), a third amino acid sequence being at least 90% homologous to the amino acid sequence VCRAGCSPEHGFCEQPGECRCLEGWTOPLCTVPVSTSSCLSPRGPSSATTGCLVPGPGPCDGN PCANGGSCSETPRSFECTCPRGFYGLRCEVSGVTCA corresponding to amino acids 219-317 of Q8NBS4_HUMAN (SEQ. ID NO: 55), also corresponding to amino acids 219-317 of N43992_P16 (SEQ. ID NO: 59), a third bridging amino acid D corresponding to amino acid 318 of N43992_P16 (SEQ. ID NO: 59), a fourth amino acid sequence being at least 90% homologous to the amino acid sequence GPCFNGGLCVGGADPDSAYICHCPPGFQGSNCEKRVDRCSLQPCRNG corresponding to amino acids 319-365 of Q8NBS4_HUMAN (SEQ. ID NO: 55), also corresponding to amino acids 319-365 of N43992_P16 (SEQ. ID NO: 59), and a fifth amino acid sequence being at least about 70%, optionally at least about 80%, preferably at least about 85%, more preferably at least about 90% and most preferably at least about 95% homologous to the amino acid sequence EAWRPERRGMGWGSWMAQTVQGWNPGFDSSNPRAWGPDLPPASL (SEQ. ID NO: 328) corresponding to amino acids 366-409 of N43992_P16 (SEQ. ID NO: 59), wherein the first amino acid sequence, first bridging amino acid, second amino acid sequence, second bridging amino acid, third amino acid sequence, third bridging amino acid, fourth amino acid sequence and fifth amino acid sequence are contiguous and in a sequential order.

According to yet other embodiments, the present invention now discloses a novel cluster designated herein D12115, comprising novel amino acid and nucleic acid sequences that are variants of the known Complement factor B precursor (CFAB_HUMAN (SEQ. ID NO: 395)). The novel polynucleotides and polypeptides described by the present invention are useful as diagnostic markers, preferably as serum markers.

Surprisingly, the present invention now shows that the D12115 variants are overexpressed in cancerous tissues, particularly in cancerous lung, cancerous breast and cancerous ovarian tissues, and thus can be used for the diagnosis, prognosis, treatment selection and treatment monitoring and/or assessment of cancers, particularly lung cancer, breast cancer and ovarian cancer, as described in a greater detail below.

According to one embodiment, the present invention provides an isolated polypeptide comprising an edge portion of D12115_P3 (SEQ. ID NO:134), comprising an amino acid sequence being at least about 70%, optionally at least about 80%, preferably at least about 85%, more preferably at least about 90% and most preferably at least about 95% homologous to the sequence AALAEGETPR (SEQ. ID NO: 329).

According to one embodiment, the isolated polypeptide is a chimeric polypeptides comprising a first amino acid sequence being at least about 90% homologous to amino acids 1-730 of CFAB_HUMAN (SEQ. ID NO: 395), also corresponding to amino acids 1-730 of D12115_P3 (SEQ. ID NO:134), and a second amino acid sequence being at least about 70%, optionally at least about 80%, preferably at least about 85%, more preferably at least about 90% and most preferably at least about 95% homologous to a polypeptide having the sequence AALAEGETPR (SEQ. ID NO: 329) corresponding to amino acids 731-740 of D12115_P3 (SEQ. ID NO:134), wherein said first amino acid sequence and second amino acid sequence are contiguous and in a sequential order.

According to one embodiment, the isolated polypeptide is a chimeric polypeptides comprising a first amino acid sequence being at least about 90% homologous to amino acids 1-31 of NP_{—}001701 (SEQ. ID NO:133), also corresponding to amino acids 1-31 of D12115_P3 (SEQ. ID NO:134), a bridging amino acid R corresponding to amino acid 32 of D12115_P3 (SEQ. ID NO:134), a second amino acid sequence being at least about 90% homologous to amino acids 33-730 of NP_{—}001701 (SEQ. ID NO:133), also corresponding to amino acids 33-730 of D12115_P3 (SEQ. ID NO:134), and a third amino acid sequence being at least about 70%, optionally at least about 80%, preferably at least about 85%, more preferably at least about 90% and most preferably at least about 95% homologous to a polypeptide having the sequence AALAEGETPR (SEQ. ID NO: 329) corresponding to amino acids 731-740 of D12115_P3 (SEQ. ID NO:134), wherein said first amino acid sequence, bridging amino acid, second amino acid sequence and third amino acid sequence are contiguous and in a sequential order.

According to one embodiment, the present invention provides an isolated polypeptide comprising an edge portion of D12115_P3 (SEQ. ID NO:134), comprising an amino acid sequence being at least about 70%, optionally at least about 80%, preferably at least about 85%, more preferably at least about 90% and most preferably at least about 95% homologous to the sequence—GEKRDLEIEVVLFHPNYNINGKKEAGIPEFYDYDVALIKLICNKLKYGQTIRPICLPCTEGTTRA LRLPPITTCQQQKEELLPAQDIKALFVSEEEKKLTRKEVYIKNGDKKGSCERDAQYAPGYDK VKDISEVVTPRFLCTGGVSPYADPNTCRGDSGGPLIVHKRSRFIQVGVISWGVVDVCKNQKRA ALAEGETPR (SEQ. ID NO: 330).

According to one embodiment, the isolated polypeptide is a chimeric polypeptides comprising a first amino acid sequence being at least about 90% homologous to amino acids 1-542 of P00751-2 (SEQ. ID NO:132), also corresponding to amino acids 1-542 of D12115_P3 (SEQ. ID NO:134), and a second amino acid sequence being at least about 70%, optionally at least about 80%, preferably at least about 85%, more preferably at least about 90% and most preferably at least about 95% homologous to a polypeptidethe having sequence GEKRDLEIEVVLFHPNYMINGICKEAGIPEFYDYDVALIKLKNKLKYGQTIRPICLPCTEGTTRA LRLPPTTTCQQQICEELLPAQDIKALFVSEEEKICLTRKEVYIKNGDKKGSCERDAQYAPGYDK VKDISEVVTPRFLCTGGVSPYADPNTCRGDSGGPLIVHKRSRFIQVGVISWGVVDVCKNQKRA ALAEGETPR (SEQ. ID NO: 330) corresponding to amino acids 543-740 of D12115_P3 (SEQ. ID NO:134), wherein said first amino acid sequence and second amino acid sequence are contiguous and in a sequential order.

In some embodiments, the isolated chimeric proteins or polypeptides of the invention may comprise an amino acid sequence corresponding to or homologous to D12115_P5 (SEQ. ID NO:135). In some embodiments, such isolated chimeric proteins or polypeptides comprise a first amino acid sequence being at least about 90% homologous to amino acids 1-390 of CFAB_HUMAN (SEQ. ID NO: 395), which also corresponds to amino acids 1-390 of D12115_P5 (SEQ. ID NO:135), and a second amino acid sequence being at least about 70%, optionally at least about 80%, preferably at least about 85%, more preferably at least about 90% and most preferably at least about 95% homologous to a polypeptide having the sequence QKGPLSCPSLPTFSDQHVALKSTCNTIPMVGALNVTHSWLFISPVTLHKEFFLSPVINYL (SEQ. ID NO: 331) corresponding to amino acids 391-450 of D12115_P5 (SEQ. ID NO:135), wherein said first amino acid sequence and second amino acid sequence are contiguous and in a sequential order.

In some embodiments, this invention provides an isolated polypeptide comprising an edge portion of D12115_P5 (SEQ. ID NO:135), comprising an amino acid sequence being at least about 70%, optionally at least about 80%, preferably at least about 85%, more preferably at least about 90% and most preferably at least about 95% homologous to the sequence—QKGPLSCPSLPTFSDQHVALKSTCNTIPMVGALNVTHSWLFISPVTLHKEFFLSPVINYL (SEQ. ID NO: 331) of D12115_P5 (SEQ. ID NO:135).

In some embodiments, such isolated chimeric proteins or polypeptides comprise a first amino acid sequence being at least about 90% homologous to amino acids 1-390 of P00751-2 (SEQ. ID NO:132), which also corresponds to amino acids 1-390 of D12115_P5 (SEQ. ID NO:135), and a second amino acid sequence being at least about 70%, optionally at least about 80%, preferably at least about 85%, more preferably at least about 90% and most preferably at least about 95% homologous to a polypeptide having the sequence QKGPLSCPSLPTFSDQHVALKSTCNTIPMVGALNVTHSWLFISPVTLHKEFFLSPVINYL (SEQ. ID NO: 331) corresponding to amino acids 391-450 of D12115_P5 (SEQ. ID NO:135), wherein sai first amino acid sequence and second amino acid sequence are contiguous and in a sequential order.

In some embodiments, this invention provides an isolated polypeptide comprising an edge portion of D12115_P5 (SEQ. ID NO:135), comprising an amino acid sequence being at least about 70%, optionally at least about 80%, preferably at least about 85%, more preferably at least about 90% and most preferably at least about 95% homologous to the sequence—QKGPLSCPSLPTFSDQHVALKSTCNTIPMVGALNVTHSWLFISPVTLHKEFFLSPVINYL (SEQ. ID NO: 331) of D12115_P5 (SEQ. ID NO:135). In some embodiments, such isolated chimeric proteins or polypeptides comprise a first amino acid sequence being at least about 90% homologous to MGSNLSPQLCLMPFILGLLSGGVTTTPWSLA corresponding to amino acids 1-31 of NP_{—}001701 (SEQ. ID NO:133), which also corresponds to amino acids 1-31 of D12115_P5 (SEQ. ID NO:135), a bridging amino acid R corresponding to amino acid 32 of D12115_P5 (SEQ. ID NO:135), a second amino acid sequence being at least about 90% homologous to amino acids 33-390 of NP_{—}001701 (SEQ. ID NO:133), which also corresponds to amino acids 33-390 of D12115_P5 (SEQ. ID NO:135), and a third amino acid sequence being at least about 70%, optionally at least about 80%, preferably at least about 85%, more preferably at least about 90% and most preferably at least about 95% homologous to a polypeptide having the sequence QKGPLSCPSLPTFSDQHVALKSTCNTIPMVGALNVTHSWLFISPVTLEKEFFLSPVINYL (SEQ. ED NO: 331) corresponding to amino acids 391-450 of D12115_P5 (SEQ. ID NO:135), wherein said first amino acid sequence, bridging amino acid, second amino acid sequence and third amino acid sequence are contiguous and in a sequential order.

In some embodiments, this invention provides an isolated polypeptide comprising an edge portion of D12115_P5 (SEQ. ID NO:135), comprising an amino acid sequence being at least about 70%, optionally at least about 80%, preferably at least about 85%, more preferably at least about 90% and most preferably at least about 95% homologous to the sequence—QKGPLSCPSLPTFSDQHVALKSTCNTIPMVGALNVTHSWLFISPVTLHKEFFLSPVINYL (SEQ. ID NO: 331) of D12115_P5 (SEQ. ID NO:135).

In some embodiments, the isolated chimeric proteins or polypeptides of the invention comprise an amino acid sequence corresponding to or homologous to D12115_P12 (SEQ. ID NO:136). In some embodiments, such isolated chimeric proteins or polypeptides comprise a first amino acid sequence being at least about 90% homologous to amino acids 1-714 of CFAB_HUMAN (SEQ. ID NO: 395), which also corresponds to amino acids 1-714 of D12115_P12 (SEQ. ID NO:136), and a second amino acid sequence being at least about 70%, optionally at least about 80%, preferably at least about 85%, more preferably at least about 90% and most preferably at least about 95% homologous to a polypeptide having the sequence SPPFPIWGDAKWSAWAPKQESSMHVASNSR (SEQ. ID NO: 332) corresponding to amino acids 715-744 of D12115_P12 (SEQ. ID NO:136), wherein said first amino acid sequence and second amino acid sequence are contiguous and in a sequential order.

In some embodiments, this invention provides an isolated polypeptide an edge portion of D12115_P12 (SEQ. ID NO:136), comprising an amino acid sequence being at least about 70%, optionally at least about 80%, preferably at least about 85%, more preferably at least about 90% and most preferably at least about 95% homologous to the sequence SPPFPIWGDAKWSAWAPKQESSMHVASNSR (SEQ. ID NO: 332) of D12115_P12 (SEQ. ID NO:136).

In some embodiments, such isolated chimeric proteins or polypeptides comprise a first amino acid sequence being at least about 90% homologous to MGSNLSPQLCLYLPFELGLLSGGVTTTPWSLA corresponding to amino acids 1-31 of NP_{—}001701 (SEQ. ID NO:133), which also corresponds to amino acids 1-31 of D12115_P12 (SEQ. ID NO:136), a bridging amino acid R corresponding to amino acid 32 of D12115_P12 (SEQ. ID NO:136), a second amino acid sequence being at least about 90% homologous to amino acids 33-714 of NP_{—}001701 (SEQ. ID NO:133), which also corresponds to amino acids 33-714 of D12115_P12 (SEQ. ID NO:136), and a third amino acid sequence being at least about 70%, optionally at least about 80%, preferably at least about 85%, more preferably at least about 90% and most preferably at least about 95% homologous to a polypeptide having the sequence SPPFPIWGDAKWSAWAPKQESSMHVASNSR (SEQ. ID NO: 332) corresponding to amino acids 715-744 of D12115_P12 (SEQ. ID NO:136), wherein said first amino acid sequence, bridging amino acid, second amino acid sequence and third amino acid sequence are contiguous and in a sequential order.

In some embodiments, this invention provides an isolated polypeptide an edge portion of D12115_P12 (SEQ. ID NO:136), comprising an amino acid sequence being at least about 70%, optionally at least about 80%, preferably at least about 85%, more preferably at least about 90% and most preferably at least about 95% homologous to the sequence SPPFPIWGDAKWSAWAPKQESSMHVASNSR (SEQ. ID NO: 332) of D12115_P12 (SEQ. ID NO:136).

In some embodiments, such isolated chimeric proteins or polypeptides comprise comprising a first amino acid sequence being at least about 90% homologous to amino acids 1-542 of P00751-2 (SEQ. ID NO:132), which also corresponds to amino acids 1-542 of D12115_P12 (SEQ. ID NO:136), and a second amino acid sequence being at least about 70%, optionally at least about 80%, preferably at least about 85%, more preferably at least about 90% and most preferably at least about 95% homologous to a polypeptide having the sequence GEKRDLEIEVVLFHPNYNINGKKEAGIPEFYDYDVALIKLKNKLKYGQTIRPICLPCTEGTTRA LRLPPTTTCQQQICEELLPAQDIKALFVSEEEKKLTRKEVYIKNGDKKGSCERDAQYAPGYDK VKDISEVVTPRFLCTGGVSPYADPNTCRGDSGGPLIVHKRSRFIQVSPPFPIWGDAKWSAWAP KQESSMHVASNSR (SEQ. ID NO: 333) corresponding to amino acids 543-744 of D12115_P12 (SEQ. ID NO:136), wherein said first amino acid sequence and second amino acid sequence are contiguous and in a sequential order.

In some embodiments, this invention provides an isolated polypeptide comprising an edge portion of D12115_P12 (SEQ. ID NO:136), comprising an amino acid sequence being at least about 70%, optionally at least about 80%, preferably at least about 85%, more preferably at least about 90% and most preferably at least about 95% homologous to the sequence—GEKRDLEIEVVLFHPNYNINGKKEAGIPEFYDYDVALIKLKNKLKYGQTIRPICLPCTEGTTRA LRLPFITTCQQQKEELLPAQDIKALFVSEEEKKLTRKEVYIICNGDICKGSCERDAQYAPGYDK VKDISEVVTPRFLCTGGVSPYADPNTCRGDSGGPLIVHKRSRFIQVSPPFPIWGDAKWSAWAP KQESSMHVASNSR (SEQ. ID NO: 333) of D12115_P12 (SEQ. ID NO:136).

In some embodiments, the isolated chimeric proteins or polypeptides of the invention comprise an amino acid sequence corresponding to or homologous to D12115_P13 (SEQ. ID NO:137).

In some embodiments, such isolated chimeric proteins or polypeptides comprise a first amino acid sequence being at least about 90% homolbgous to amino acids 1-618 of CFAB_HUMAN (SEQ. ID NO: 395), which also corresponds to amino acids 1-618 of D12115_P13 (SEQ. ID NO:137), and a second amino acid sequence being at least about 70%, optionally at least about 80%, preferably at least about 85%, more preferably at least about 90% and most preferably at least about 95% homologous to a polypeptide having the sequence RRAAPCTGYQSSVCV (SEQ. ID NO: 334) corresponding to amino acids 619-633 of D12115_P13 (SEQ. ID NO:137), wherein said first amino acid sequence and second amino acid sequence are contiguous and in a sequential order.

In some embodiments, this invention provides an isolated polypeptide comprising an edge portion of D12115_P13 (SEQ. ID NO:137), comprising an amino acid sequence being at least about 70%, optionally at least about 80%, preferably at least about 85%, more preferably at least about 90% and most preferably at least about 95% homologous to the sequenCe RRAAPCTGYQSSVCV (SEQ. ID NO: 334) of D12115_P13 (SEQ. ID NO:137).

In some embodiments, such isolated chimeric proteins or polypeptides comprise a first amino acid sequence being at least about 90% homologous to amino acids 1-31 of NP_{—}001701 (SEQ. ID NO:133), which also corresponds to amino acids 1-31 of D12115_P13 (SEQ. ID NO:137), a bridging amino acid R corresponding to amino acid 32 of D12115_P13 (SEQ. ID NO:137), a second amino acid sequence being at least about 90% homologous to amino acids 33-618 of NP_{—}001701 (SEQ. ID NO:133), which also corresponds to amino acids 33-618 of D12115_P13 (SEQ. ID NO:137), and a third amino acid sequence being at least about 70%, optionally at least about 80%, preferably at least about 85%, more preferably at least about 90% and most preferably at least about 95% homologous to a polypeptide having the sequence RRAAPCTGYQSSVCV (SEQ. ID NO: 334) corresponding to amino acids 619-633 of D12115_P13 (SEQ. ID NO:137), wherein said first amino acid sequence, bridging amino acid, second amino acid sequence and third amino acid sequence are contiguous and in a sequential order.

In some embodiments, this invention provides an isolated polypeptide comprising an edge portion of D12115_P13 (SEQ. ID NO:137), comprising an amino acid sequence being at least about 70%, optionally at least about 80%, preferably at least about 85%, more preferably at least about 90% and most preferably at least about 95% homologous to the sequence RRAAPCTGYQSSVCV (SEQ. ID NO: 334) of D12115_P13 (SEQ. ID NO:137).

In some embodiments, such isolated chimeric proteins or polypeptides comprise a first amino acid sequence being at least about 90% homologous to amino acids 1-542 of P00751-2 (SEQ. ID NO:132), which also corresponds to amino acids 1-542 of D12115_P13 (SEQ. ID NO:137), and a second amino acid sequence being at least about 70%, optionally at least about 80%, preferably at least about 85%, more preferably at least about 90% and most preferably at least about 95% homologous to a polypeptide having the sequence GEKRDLELEVVLFHPNYNINGKKEAGIPEFYDYDVALIKLKNKLKYGQTIRPICLPCTEGTTRA LRLPPTTTCQQQRRAAPCTGYQSSVCV (SEQ. ID NO: 335) corresponding to amino acids 543-633 of D12115_P13 (SEQ. ID NO:137), wherein said first amino acid sequence and second amino acid sequence are contiguous and in a sequential order.

In some embodiments, this invention provides an isolated polypeptide comprising an edge portion of D12115_P13 (SEQ. ID NO:137), comprising an amino acid sequence being at least about 70%, optionally at least about 80%, preferably at least about 85%, more preferably at least about 90% and most preferably at least about 95% homologous to the sequence—GEKRDLEIEVVLFHPNYNINGKICEAGEPEFYDYDVALIKLKNKLKYGQTIRPICLPCTEGTTRA LRLPPTTTCQQQRRAAPCTGYQSSVCV (SEQ. ID NO: 335) of D12115_P13 (SEQ. ID NO:137).

In some embodiments, the isolated chimeric proteins or polypeptides of the invention comprise an amino acid sequence corresponding to or homologous to D12115_P15 (SEQ. ID NO:138).

In some embodiments, such isolated chimeric proteins or polypeptides comprise a first amino acid sequence being at least about 90% homologous to amino acids 1-593 of CFAB_HUMAN (SEQ. ID NO: 395), which also corresponds to amino acids 1-593 of D12115_P15 (SEQ. ID NO:138), and a second amino acid sequence being at least about 70%, optionally at least about 80%, preferably at least about 85%, more preferably at least about 90% and most preferably at least about 95% homologous to a polypeptide having the sequence GRAAPCTGYQSSVCV (SEQ. ID NO: 336) corresponding to amino acids 594-608 of D12115_P15 (SEQ. ID NO:138), wherein said first amino acid sequence and second amino acid sequence are contiguous and in a sequential order.

In some embodiments, this invention provides an isolated polypeptide comprising an edge portion of D12115_P15 (SEQ. ID NO:138), comprising an amino acid sequence being at least about 70%, optionally at least about 80%, preferably at least about 85%, more preferably at least about 90% and most preferably at least about 95% homologous to the sequence GRAAPCTGYQSSVCV (SEQ. ID NO: 336) of D12115_P15 (SEQ. ID NO:138).

In some embodiments, such isolated chimeric proteins or polypeptides comprise a first amino acid sequence being at least about 90% homologous to amino acids 1-31 of NP_{—}001701 (SEQ. ID NO:133), which also corresponds to amino acids 1-31 of D12115_P15 (SEQ. ID NO:138), a bridging amino acid R corresponding to amino acid 32 of D12115_P15 (SEQ. ID NO:138), a second amino acid sequence being at least about 90% homologous to amino acids 33-593 of NP_{—}001701 (SEQ. ID NO:133), which also corresponds to amino acids 33-593 of D12115_P15 (SEQ. ID NO:138), and a third amino acid sequence being at least about 70%, optionally at least about 80%, preferably at least about 85%, more preferably at least about 90% and most preferably at least about 95% homologous to a polypeptide having the sequence GRAAPCTGYQSSVCV (SEQ. ID NO: 336) corresponding to amino acids 594-608 of D12115_P15 (SEQ. ID NO:138), wherein said first amino acid sequence, bridging amino acid, second amino acid sequence and third amino acid sequence are contiguous and in a sequential order.

In some embodiments, this invention provides an isolated polypeptide an edge portion of D12115_P15 (SEQ. ID NO:138), comprising an amino acid sequence being at least about 70%, optionally at least about 80%, preferably at least about 85%, more preferably at least about 90% and most preferably at least about 95% homologous to the sequence GRAAPCTGYQSSVCV (SEQ. ID NO: 336) of D12115_P15 (SEQ. ID NO:138).

In some embodiments, such isolated chimeric proteins or polypeptides comprise a first amino acid sequence being at least about 90% homologous to amino acids 1-542 of P00751-2 (SEQ. ID NO:132), which also corresponds to amino acids 1-542 of D12115_P15 (SEQ. ID NO:138), and a second amino acid sequence being at least about 70%, optionally at least about 80%, preferably at least about 85%, more preferably at least about 90% and most preferably at least about 95% homologous to a polypeptide having the sequence GEICRDLEIEVVLFHPNYNINGICKEAGEPEFYDYDVALIKLKNKLKYGQTIRGRAAPCTGYQSS VCV (SEQ. ID NO: 337) corresponding to amino acids 543-608 of D12115_P15 (SEQ. ID NO:138), wherein said first amino acid sequence and second amino acid sequence are contiguous and in a sequential order.

In some embodiments, this invention provides an isolated polypeptide comprising an edge portion of D12115_P15 (SEQ. ID NO:138), comprising an amino acid sequence being at least about 70%, optionally atleast about 80%, preferably at least about 85%, more preferably at least about 90% and most preferably at least about 95% homologous to the sequence—GEKRDLEILEVVLFHPNYNINGICKEAGIPEFYDYDVALIKLKNKLKYGOIRGRAAPCTGYQSS VCV (SEQ. ID NO: 337) of D12115_P15 (SEQ. ID NO:138).

In some embodiments, the isolated chimeric proteins or polypeptides of the invention comprise an amino acid sequence corresponding to or homologous to D12115_P16 (SEQ. ID NO:139). In some embodiments, such isolated chimeric proteins or polypeptides comprise a first amino acid sequence being at least about 90% homologous to amino acids 1-652 of CFAB_HUMAN (SEQ. ID NO: 395), which also corresponds to amino acids 1-652 of D12115_P16 (SEQ. ID NO:139), and a second amino acid sequence being at least about 70%, optionally at least about 80%, preferably at least about 85%, more preferably at least about 90% and most preferably at least about 95% homologous to a polypeptide having the sequence VRNGHPKEAL (SEQ. ID NO: 338) corresponding to amino acids 653-662 of D12115_P16 (SEQ. ID NO:139), wherein said first amino acid sequence and second amino acid sequence are contiguous and in a sequential order.

In some embodiments, this invention provides an isolated polypeptide comprising an edge portion of D12115_P16 (SEQ. ID NO:139), comprising an amino acid sequence being at least about 70%, optionally at least about 80%, preferably at least about 85%, more preferably at least about 90% and most preferably at least about 95% homologous to the sequence VRNGHPKEAL (SEQ. ID NO: 338) of D12115_P16 (SEQ. ID NO:139).

In some embodiments, such isolated chimeric proteins or polypeptides comprise a first amino acid sequence being at least about 90% homologous to amino acids 1-31 of NP_{—}001701 (SEQ. ID NO:133), which also corresponds to amino acids 1-31 of D12115_P16 (SEQ. ID NO:139), a bridging amino acid R corresponding to amino acid 32 of D12115_P16 (SEQ. ID NO:139), a second amino acid sequence being at least about 90% homologous to amino acids 33-652 of NP_{—}001701 (SEQ. ID NO:133), which also corresponds to amino acids 33-652 of D12115_P16 (SEQ. ID NO:139), and a third amino acid sequence being at least about 70%, optionally at least about 80%, preferably at least about 85%, more preferably at least about 90% and most preferably at least about 95% homologous to a polypeptide having the sequence VRNGHPKEAL (SEQ. ID NO: 338) corresponding to amino acids 653-662 of D12115_P16 (SEQ. ID NO:139), wherein said first amino acid sequence, bridging amino acid, second amino acid sequence and third amino acid sequence are contiguous and in a sequential order.

In some embodiments, this invention provides an isolated polypeptide comprising an edge portion of D12115_P16 (SEQ. ID NO:139), comprising an amino acid sequence being at least about 70%, optionally at least about 80%, preferably at least about 85%, more preferably at least about 90% and most preferably at least about 95% homologous to the sequence VRNGHPKEAL (SEQ. ID NO: 338) of D12115_P16 (SEQ. ID NO:139).

In some embodiments, such isolated chimeric proteins or polypeptides comprise a first amino acid sequence being at least about 90% homologous to amino acids 1-542 of P00751-2 (SEQ. ID NO:132), which also corresponds to amino acids 1-542 of D12115_P16 (SEQ. ID NO:139), and a second amino acid sequence being at least about 70%, optionally at least about 80%, preferably at least about 85%, more preferably at least about 90% and most preferably at least about 95% homologous to a polypeptide having the sequence GEKRDLEIEVVLFHPNYNINGKKEAGIPEFYDYDVALIKLKNKLKYGQTIRPICLPCTEGTTRA LRLPPTTTCQQQKEELLPAQDTKALFVSEEEKKLTRKEVYIKNGDKVRNGHPKEAL (SEQ. ID NO: 339) corresponding to amino acids 543-662 of D12115_P16 (SEQ. ID NO:139), wherein said first amino acid sequence and second amino acid sequence are contiguous and in a sequential order.

In some embodiments, this invention provides an isolated polypeptide comprising an edge portion of D12115_P16 (SEQ. ID NO:139), comprising an amino acid sequence being at least about 70%, optionally at least about 80%, preferably at least about 85%, more preferably at least about 90% and most preferably at least about 95% homologous to the sequence—GEKRDLELEVVLFHPNYNINGKICEAGIPEFYDYDVALJKLICNICLKYGQTIRPICLPCTEGTTRA LRLPPTTTCQQQKEELLPAQDIKALFVSEEEICKLTRICEVYLKNGDKVRNGHPKEAL (SEQ. ID NO: 339) of D12115_P16 (SEQ. ID NO:139).

In some embodiments, the isolated chimeric proteins or polypeptides of the invention comprise an amino acid sequence corresponding to or homologous to D12115_P20 (SEQ. ID NO:140).

In some embodiments, such isolated chimeric proteins or polypeptides comprise a first amino acid sequence being at least about 90% homologous to amino acids 1-541 of CFAB_HUMAN (SEQ. ID NO: 395), which also corresponds to amino acids 1-541 of D12115_P20 (SEQ. ID NO:140), a second bridging amino acid sequence comprising of E, and a third amino acid sequence being at least about 90% homologous to amino acids 620-764 of CFAB_HUMAN (SEQ. ID NO: 395), which also corresponds to amino acids 543-687 of D12115_P20 (SEQ. ID NO:140), wherein said first amino acid sequence, second amino acid sequence and third amino acid sequence are contiguous and in a sequential order.

In some embodiments, this invention provides an isolated polypeptide comprising an edge portion of D12115_P20 (SEQ. ID NO:140), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least 3 amino acids comprise VEE having a structure as follows (numbering according to D12115_P20 (SEQ. ID NO:140)): a sequence starting from any of amino acid numbers 541-x to 541; and ending at any of amino acid numbers 543+((n−3)−x), in which x varies from 0 to n−3.

In some embodiments, such isolated chimeric proteins or polypeptides comprise a first amino acid sequence being at least about 90% homologous to amino acids 1-31 of NP_{—}001701 (SEQ. ID NO:133), which also corresponds to amino acids 1-31 of D12115_P20 (SEQ. ID NO:140), a bridging amino acid R corresponding to amino acid 32 of D12115_P20 (SEQ. ID NO:140), a second amino acid sequence being at least about 90% homologous to amino acids 33-541 of NP_{—}001701 (SEQ. ID NO:133), which also corresponds to amino acids 33-541 of D12115_P20 (SEQ. ID NO:140), a third bridging amino acid sequence comprising of E, and a fourth amino acid sequence being at least about 90% homologous to amino acids 620-764 of NP_{—}001701 (SEQ. ID NO:133), which also corresponds to amino acids 543-687 of D12115_P20 (SEQ. ID NO:140), wherein said first amino acid sequence, bridging amino acid, second amino acid sequence, third amino acid sequence and fourth amino acid sequence are contiguous and in a sequential order.

In some embodiments, such isolated chimeric proteins or polypeptides comprise a first amino acid sequence being at least about 90% homologous to amino acids 1-541 of P00751-2 (SEQ. ID NO:132), which also corresponds to amino acids 1-541 of D12115_P20 (SEQ. ID NO:140), and a second amino acid sequence being at least about 70%, optionally at least about 80%, preferably at least about 85%, more preferably at least about 90% and most preferably at least about 95% homologous to a polypeptide having the sequence EEELLPAMICALFVSEEEICKLTRICEVYIKNGDICKGSCERDAQYAPGYDKVICDISEVVTPRFL CTGGVSPYADPNTCRGDSGGPLIVHECRSRFIQVGVISWGVVDVCICNQKRQKQVPAHARDFHI NLFQVLPWLKEKLQDEDLGFL (SEQ. ID NO: 340) corresponding to amino acids 542-687 of D12115_P20 (SEQ. ID NO:140), wherein said first amino acid sequence and second amino acid sequence are contiguous and in a sequential order.

In some embodiments, this invention provides an isolated polypeptide comprising an edge portion of D12115_P20 (SEQ. ID NO:140), comprising an amino acid sequence being at least about 70%, optionally at least about 80%, preferably at least about 85%, more preferably at least about 90% and most preferably at least about 95% homologous to the sequence—EEELLPAQDIKALFVSEEEKKLTRKEVYIKNGDKKGSCERDAQYAPGYDKVKDISEVVTPRFL CTGGVSPYADPNTCRGDSGGPLIVHKRSRFIQVGVISWGVVDVCKNQKRQKQVPAHARDFHE NLFQVLPWLKEKLQDEDLGFL (SEQ. ID NO: 340) of D12115_P20 (SEQ. ID NO:140).

In some embodiments, the isolated chimeric proteins or polypeptides of the invention comprise an amino acid sequence corresponding to or homologous to D12115_P32 (SEQ. ID NO:141). In some embodiments, such isolated chimeric proteins or polypeptides comprise a first amino acid sequence being at least about 90% homologous to amino acids 1-469 of CFAB_HUMAN (SEQ. ID NO: 395), which also corresponds to amino acids 1-469 of D12115_P32 (SEQ. ID NO:141), and a second amino acid sequence being at least about 70%, optionally at least about .80%, preferably at least about 85%, more preferably at least about 90% and most preferably at least about 95% homologous to a polypeptide having the sequence GREIQGNKEHNS (SEQ. ID NO: 341) corresponding to amino acids 470-481 of D12115_P32 (SEQ. ID NO:141), wherein said first amino acid sequence and second amino acid sequence are contiguous and in a sequential order.

In some embodiments, this invention provides an isolated polypeptide comprising an edge portion of D12115_P32 (SEQ. ID NO:141), comprising an amino acid sequence being at least about 70%, optionally at least about 80%, preferably at least about 85%, more preferably at least about 90% and most preferably at least about 95% homologous to the sequence GREIQGNKEHNS (SEQ. ID NO: 341) of D12115_P32 (SEQ. ID NO:141).

In some embodiments, such isolated chimeric proteins or polypeptides comprise a first amino acid sequence being at least about 90% homologous to amino acids 1-469 of P00751-2 (SEQ. ID NO:132), which also corresponds to amino acids 1-469 of D12115_P32 (SEQ. ID NO:141), and a second amino acid sequence being at least about 70%, optionally at least about 80%, preferably at least about 85%, more preferably at least about 90% and most preferably at least about 95% homologous to a polypeptide having the sequence GREIQGNKEHNS (SEQ. ID NO: 341) corresponding to amino acids 470-481 of D12115_P32 (SEQ. ID NO:141), wherein said first amino acid sequence and second amino acid sequence are contiguous and in a sequential order.

In some embodiments, this invention provides an isolated polypeptide comprising an edge portion of D12115_P32 (SEQ. ID NO:141), comprising an amino acid sequence being at least about 70%, optionally at least about 80%, preferably at least about 85%, more preferably at least about 90% and most preferably at least about 95% homologous to the sequence GREIQGNKEHNS (SEQ. ID NO: 341) of D12115_P32 (SEQ. ID NO:141).

In some embodiments, such isolated chimeric proteins or polypeptides comprise a first amino acid sequence being at least about 90% homologous to amino acids 1-31 of NP_{—}001701 (SEQ. ID NO:133), which also corresponds to amino acids 1-31 of D12115_P32 (SEQ. ID NO:141), a bridging amino acid R corresponding to amino acid 32 of D12115_P32 (SEQ. ID NO:141), a second amino acid sequence being at least about 90% homologous to amino acids 33-469 of NP_{—}001701 (SEQ. ID NO:133), which also corresponds to amino acids 33-469 of D12115_P32 (SEQ. ID NO:141), and a third amino acid sequence being at least about 70%, optionally at least about 80%, preferably at least about 85%, more preferably at least about 90% and most preferably at least about 95% homologous to a polypeptide having the sequence GREIQGNKEHNS (SEQ. ID NO: 341) corresponding to amino acids 470-481 of D12115_P32 (SEQ. ID NO:141), wherein said first amino acid sequence, bridging amino acid, second amino acid sequence and third amino acid sequence are contiguous and in a sequential order.

In some embodiments, this invention provides an isolated polypeptide comprising an edge portion of D12115_P32 (SEQ. ID NO:141), comprising an amino acid sequence being at least about 70%, optionally at least about 80%, preferably at least about 85%, more preferably at least about 90% and most preferably at least about 95% homologous to the sequence GREIQGNKEHNS (SEQ. ID NO: 341) ofD12115_P32 (SEQ. ID NO:141).

According to a further embodiment, the present invention now discloses a novel cluster designated herein C03950, comprising novel amino acid and nucleic acid sequences that are variants of the known protein Serine/threonine-protein kinase TNNI3K (SwissProt accession identifier TNI3K_HUMAN (SEQ. ID NO: 396).

The novel polynucleotides and polypeptides described by the present invention are useful as diagnostic markers, preferably as serum markers.

Surprisingly, the present invention now shows that the C03950 variants are expressed specifically in heart tissue, and thus can indicate the onset, severity or prognosis of cardiovascular disease in a subject, and can be used for the selection of treatment, treatment monitoring, diagnosis or prognosis assessment of any cardiovascular disease, including, inter alia, myocardial infarct, acute coronary syndrome, coronary artery disease, angina pectoris (stable and unstable), cardiomyopathy, myocarditis, congestive heart failure or any type of heart failure, reinfarction, assessment of thrombolytic therapy, assessment of myocardial infarct size, differential diagnosis between heart-related versus lung-related conditions (such as pulmonary embolism), the differential diagnosis of Dyspnea, cardiac valves related conditions, vascular disease, or any combination thereof, as is described in a greater detail below.

In some embodiments, the isolated chimeric proteins or polypeptides of the invention comprise an amino acid sequence corresponding to or homologous to C03950_{—}3_P5 (SEQ. ID NO:212).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence AVRRGLREGGA (SEQ. ID NO: 342) corresponding to amino acids 1-11 of C03950_{—}3_P5 (SEQ. ID NO:212), a second amino acid sequence being at least about 90% homologous to amino acids 1-691 of TNI3K_HUMAN (SEQ. ID NO: 396), which also corresponds to amino acids 12-702 of C03950_{—}3_P5 (SEQ. ID NO:212), a third amino acid sequence being at least about 70%, optionally at least about 80%, preferably at least about 85%, more preferably at least about 90% and most preferably at least about 95%, homologous to a polypeptide having the sequence RSAITSRIWITHSICIWRGAHYFNREECNFRCMLTSAILK corresponding to amino acids 703-742 of C03950_{—}3_P5 (SEQ. ID NO:212), and a fourth amino acid sequence being at least about 90% homologous to amino acids 710-936 of TNI3K_HUMAN (SEQ. ID NO: 396), which also corresponds to amino acids 743-969 of C03950_{—}3_P5 (SEQ. ID NO:212), wherein said first amino acid sequence, second amino acid sequence, third amino acid sequence and fourth amino acid sequence are contiguous and in a sequential order.

In some embodiments, this invention provides an isolated polypeptide comprising a head of C03950_{—}3_P5 (SEQ. ID NO:212), comprising a polypeptide being at least about 70%, optionally at least about 80%, preferably at least about 85%, more preferably at least about 90% and most preferably at least about 95% homologous to the sequence AVRRGLREGGA (SEQ. ID NO: 342) of C03950_{—}3_P5 (SEQ. ID NO:212).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence—AVRRGLREGGAMAAARDPPEVSLREATQRICLRRFSELRGKLVARGEFWDIVAITAADEKQEL AYNQQLSEICLKRICELPLGVQYHVFVDPAGAKIGNGGSTLCALQCLEKLYGDKWNSFTILLIH S (SEQ. ID NO: 343) corresponding to amino acids 1-125 of C03950_{—}3_P5 (SEQ. ID NO:212), second amino acid sequence being at least about 90% homologous to amino acids 14-590 of NP_{—}057062 (SEQ. ID NO:210), which also corresponds to amino acids 126-702 of C03950_{—}3_P5 (SEQ. ID NO:212), a third amino acid sequence being at least about 70%, optionally at least about 80%, preferably at least about 85%, more preferably at least about 90% and most preferably at least about 95%, homologous to a polypeptide having the sequence RSAITSRIWITHSICIWRGAHYFNREECNFRCMLTSAILK corresponding to amino acids 703-742 of C03950_{—}3_P5 (SEQ. ID NO:212), and a fourth amino acid sequence being at least about 90% homologous to amino acids 609-835 of NP_{—}057062 (SEQ. ID NO:210), which also corresponds to amino acids 743-969 of C03950_{—}3_P5 (SEQ. ID NO:212), wherein said first amino acid sequence, second amino acid sequence, third amino acid sequence and fourth amino acid sequence are contiguous and in a sequential order.

In some embodiments, this invention provides an isolated polypeptide comprising a head of C03950_{—}3_P5 (SEQ. ID NO:212), comprising a polypeptide being at least about 70%, optionally at least about 80%, preferably at least about 85%, more preferably at least about 90% and most preferably at least about 95% homologous to the sequence AVRRGLREGGAMAAARDPPEVSLIZEATQRKLRRFSELRGICLVARGEFWDIVAITAADEKQEL AYNQQLSEKLKRKELPLGVQYHVFVDPAGAKIGNGGSTLCALQCLEKLYGDICWNSFTILLIH S (SEQ. ID NO: 343) of C03950_{—}3_P5 (SEQ. ID NO:212).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence—AVRRGLREGGAMAAARDPPEVSLREATQRKLRRFSELRGKLVARGEFWDIVAITAADEKQEL AYNQQLSEKLKRKELPLGVQYHVFVDPAGAKIGNGGSTLCALQCLEKLYGDKWNSFTILLIH S (SEQ. ID NO: 343) corresponding to amino acids 1-125 of C03950_{—}3_P5 (SEQ. ID NO:212), a second amino acid sequence being at least about 90% homologous to amino acids 14-590 of Q9Y2V6_HUMAN (SEQ. ID NO:210), which also corresponds to amino acids 126-702 of C03950_{—}3_P5 (SEQ. ID NO:212), a third amino acid sequence being at least about 70%, optionally at least about 80%, preferably at least about 85%, more preferably at least about. 90% and most preferably at least about 95%, homologous to a polypeptide having the sequence RSAITSRIWITHSICIWRGAHYPNREECNFRCMLTSAILK corresponding to amino acids 703-742 of C03950_{—}3_P5 (SEQ. ID NO:212), and a fourth amino acid sequence being at least about 90% homologous to amino acids 609-835 of Q9Y2V6_HUMAN (SEQ. ID NO:210), which also corresponds to amino acids 743-969 of C03950_{—}3_P5 (SEQ. ID NO:212), wherein said first amino acid sequence, second amino acid sequence, third amino acid sequence and fourth amino acid sequence are contiguous and in a sequential order.

In some embodiments, this invention provides an isolated polypeptide comprising a head of C03950_{—}3_P5 (SEQ. ID NO:212), comprising a polypeptide being at least about 70%, optionally at least about 80%, preferably at least about 85%, more preferably at least about 90% and most preferably at least about 95% homologous to the sequence AVRRGLREGGAMAAARDPPEVSLREATQRKLRRFSELRGKLVARGEFWDIVAITAADEKQEL AYNQQLSEKLKRKELPLGVQYHVFVDPAGAKIGNGGSTLCALQCLEKLYGDKWNSFTILLIH S (SEQ. ID NO: 343) of C03950_{—}3_P5 (SEQ. ID NO:212).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence AVRRGLR (SEQ. ID NO: 344) corresponding to amino acids 1-7 of C03950_{—}3_P5 (SEQ. ID NO:212), a second amino acid sequence being at least about 90% homologous to amino acids 14-367 of Q6MZS9_HUMAN (SEQ.' ID NO:211), which also corresponds to amino acids 8-361 of C03950_{—}3_P5 (SEQ. ID NO:212), a bridging amino acid I corresponding to amino acid 362 of C03950_{—}3_P5 (SEQ. ID NO:212), a third amino acid sequence being at least about 90% homologous to amino acids 369-484 of Q6MZS9_HUMAN (SEQ. ID NO:211), which also corresponds to amino acids 363-478 of C03950_{—}3_P5 (SEQ. ID NO:212), bridging amino acid N corresponding to amino acid 479 of C03950_{—}3_P5 (SEQ. ID NO:212), a fourth amino acid sequence being at least about 90% homologous to amino acids 486-709 of Q6MZS9_HUMAN (SEQ. ID NO:211), which also corresponds to amino acids 480-703 of C03950_{—}3_P5 (SEQ. ID NO:212), and a fifth amino acid sequence being at least about 70%, optionally at least about 80%, preferably at least about 85%, more preferably at least about 90% and most preferably at least about 95% homologous to a polypeptide having the sequence SAITSRIWITHSICIWRGAHYFNREECNFRCMLTSAILKESRFLQSLDEDNMTKQPGNLRWMA PEVFTQCTRYTIKADVFSYALCLWEILTGEIPFAHLKPAAAAADMAYHORPPIGYSIPKPISSL LIRGWNACPEGRPEFSEVVMKLEECLCNIELMSPASSNSSGSLSPSSSSDCLVNRGGPGRSHVA ALRSRFELEYALNARSYAALSQSAGQYSSQGLSLEEMKRSLQYTPIDKYGYVSDPMSSMETH SCRNSSSFEDSS (SEQ. ID NO: 345) corresponding to amino acids 704-969 of C03950_{—}3_P5 (SEQ. ID NO:212), wherein said first amino acid sequence, second amino acid sequence, bridging amino acid, third amino acid sequence, bridging amino acid, fourth amino acid sequence and fifth amino acid sequence are contiguous and in a sequential order.

In some embodiments, this invention provides an isolated polypeptide comprising a head of C03950_{—}3_P5 (SEQ. ID NO:212), comprising a polypeptide being at least about 70%, optionally at least about 80%, preferably at least about 85%, more preferably at least about 90% and most preferably at least about 95% homologous to the sequence AVRRGLR (SEQ. ID NO: 344) of C03950_{—}3_P5 (SEQ. ID NO:212).

In some embodiments, this invention provides an isolated polypeptide comprising an edge portion of C03950_{—}3_P5 (SEQ. ID NO:212), comprising an amino acid sequence being at least about 70%, optionally at least about 80%, preferably at least about 85%, more preferably at least about 90% and most preferably at least about 95% homologous to the sequence—SAITSRIWITHSICIWRGAHYFNREECNFRCMLTSAILICESRFLQSLDEDNMTKQPGNLRWMA PEVETQCTRYTIKADVFSYALCLWEILTGEIPFAHLKPAAAAADMAYEHIRPPIGYSIPKPISSL LIRGWNACPEGRPEFSEVVMKLEECLCNIELMSPAS SNS SGSL SPS SSSDCLVNRGGPGRSHVA ALRSRFELEYALNARSYAALSQSAGQYSSQGLSLEEMKRSLQYTPIDKYGYVSDPMSSMHFH SCRNSSSFEDSS (SEQ. ID NO: 345) of C03950_{—}3_P5 (SEQ. ID NO:212).

In some embodiments, the isolated chimeric proteins or polypeptides of the invention comprise an amino acid sequence corresponding to or homologous to C03950_{—}3_P7 (SEQ. ID NO:213).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MGNYKSRPTQTCT (SEQ. ID NO: 346) corresponding to amino acids 1-13 of C03950_{—}3_P7 (SEQ. ID NO:213), a second amino acid sequence being at least about 90% homologous to amino acids 115-691 of TNI3K_HUMAN (SEQ. ID NO: 396), which also corresponds to amino acids 14-590 of C03950_{—}3_P7 (SEQ. ID NO:213), a third amino acid sequence being at least about 70%, optionally at least about 80%, preferably at least about 85%, more preferably at least about 90% and most preferably at least about 95%, homologous to a polypeptide having the sequence RSAITSRIWITHSICIVVRGAHYFNREECNFRCMLTSAILK corresponding to amino acids 591-630 of C03950_{—}3_P7 (SEQ. ID NO:213), and a fourth amino acid sequence being at least about 90% homologous to amino acids 710-936 of TNI3K_HUMAN (SEQ. ID NO: 396), which also corresponds to amino acids 631-857 of C03950_{—}3_P7 (SEQ. ID NO:213), wherein said first amino acid sequence, second amino acid sequence, third amino acid sequence and fourth amino acid sequence are contiguous and in a sequential order.

In some embodiments, this invention provides an isolated polypeptide comprising a head of C03950_{—}3_P7 (SEQ. ID NO:213), comprising a polypeptide being at least about 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P7 (SEQ. ID NO:213).

In some embodiments, such isolated chimeric proteins or polypeptides comprise a first amino acid sequence being at least about 90% homologous to amino acids 1-590 of NP_{—}057062 (SEQ. ID NO:210), which also corresponds to amino acids 1-590 of C03950_{—}3_P7 (SEQ. ID NO:213), a second amino acid sequence being at least about 70%, optionally at least about 80%, preferably at least about 85%, more preferably at least about 90% and most preferably at least about 95%, homologous to a polypeptide having the sequence RSAITSRIWITHSICIWRGAHYFNREECNFRCMLTSAELK corresponding to amino acids 591-630 of C03950_{—}3_P7 (SEQ. ID NO:213), and a third amino acid sequence being at least about 90% homologous to amino acids 609-835 of NP_{—}057062 (SEQ. ID NO:210), which also corresponds to amino acids 631-857 of C03950_{—}3_P7 (SEQ. ID NO:213), wherein said first amino acid sequence, second amino acid sequence and third amino acid sequence are contiguous and in a sequential order.

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MGNYKSRPTQTCT (SEQ. ID NO: 346) corresponding to amino acids 1-13 of C03950_{—}3_P7 (SEQ. ID NO:213), a second amino acid sequence being at least about 90% homologchis to amino acids 132-367 of Q6MZS9_HUMAN (SEQ. ID NO:211), which also corresponds to amino acids 14-249 of C03950_{—}3_P7 (SEQ. ID NO:213), a bridging amino acid I corresponding to amino acid 250 of C03950_{—}3_P7 (SEQ. ID NO:213), a third amino acid sequence being at least about 90% homologous to amino acids 369-484 of Q6MZS9_HUMAN (SEQ. ID NO:211), which also corresponds to amino acids 251-366 of C03950_{—}3_P7 (SEQ. ID NO:213), bridging amino acid N corresponding to amino acid 367 of C03950_{—}3_P7 (SEQ. ID NO:213), a fourth amino acid sequence being at least about 90% homologous to amino acids 486-709 of Q6MZS9_HUMAN (SEQ. ID NO:211), which also corresponds to amino acids 368-591 of C03950_{—}3_P7 (SEQ. ID NO:213), and a fifth amino acid sequence being at least about 70%, optionally at least about 80%, preferably at least about 85%, more preferably at least about 90% and most preferably at least about 95% homologous to a polypeptide having the sequence SAITSRIWITHSICIWRGAHYFNREECNFRCMLTSAILKESRFLQSLDEDNMTKQPGNLRWMA PEVFTQCTRYTIKADVFSYALCLWEILTGEIPFAHLKPAAAAADMAYHHIRPPIGYSEPKPISSL LIRGWNACPEGRPEFSEVVMICLEECLCNIELMSPASSNSSGSLSPSSSSDCLVNRGGPGRSHVA ALRSRFELEYALNARSYAALSQSAGQYSSQGLSLEEMICRSLQYTPIDKYGYVSDPMSSIVIEFH SCRNSSSFEDSS (SEQ. ID NO: 345) corresponding to amino acids 592-857 of C03950_{—}3_P7 (SEQ. ID NO:213), wherein said first amino acid sequence, second amino acid sequence, bridging amino acid, third amino acid sequence, bridging amino acid, fourth amino acid sequence and fifth amino acid sequence are contiguous and in a sequential order.

In some embodiments, this invention provides an isolated polypeptide comprising a head of C03950_{—}3_P7 (SEQ. ID NO:213), comprising a polypeptide being at least about 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P7 (SEQ. ID NO:213).

In some embodiments, this invention provides an isolated polypeptide comprising an edge portion of C03950_{—}3_P7 (SEQ. ID NO:213), comprising an amino acid sequence being at least about 70%, optionally at least about 80%, preferably at least about 85%, more preferably at least about 90% and most preferably at least about 95% homologous to the sequence—SAITSRIWITHSICIWRGAHYFNREECNFRCMLTSAILKESRFLQSLDEDNMTKQPGNLRWMA PEVFTQCTRYTIKADVFSYALCLWElLTGELPFAHLKPAAAAADMAYHHIRPPIGYSIPICPISSL LIRGWNACPEGRPEFSEVVMKLEECLCNIELMSPASSNSSGSLSPSSSSDCLVNRGGPGRSHVA ALRSRFELEYALNARSYAALSQSAGQYSSQGLSLEEMKRSLQYTPIDKYGYVSDPMSSMHFH SCRNSSSFEDSS (SEQ. ID NO: 345) of C03950_{—}3_P7 (SEQ. ID NO:213).

In some embodiments, the isolated chimeric proteins or polypeptides of the invention comprise an amino acid sequence corresponding to or homologous to C03950_{—}3_P9 (SEQ. ID NO:214). In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MGNYKSRPTQTCT (SEQ. ID NO: 346) corresponding to amino acids 1-13 of C03950_{—}3_P9 (SEQ. ID NO:214), a second amino acid sequence being at least about 90% homologous to amino acids 115-911 of TNI3K_HUMAN (SEQ. ID NO: 396), which also corresponds to amino acids 14-810 of C03950_{—}3_P9 (SEQ. ID NO:214), and a third amino acid sequence being at least about 70%, optionally at least about 80%, preferably at least about 85%, more preferably at least about 90% and most preferably at least about 95% homologous to a polypeptide having the sequence DVTS (SEQ. ID NO: 349) corresponding to amino acids 811-814 of C03950_{—}3_P9 (SEQ. ID NO:214), wherein said first amino acid sequence, second amino acid sequence and third amino acid sequence are contiguous and in a sequential order.

In some embodiments, this invention provides an isolated polypeptide comprising a head of C03950_{—}3_P9 (SEQ. ID NO:214), comprising a polypeptide being at least about 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P9 (SEQ. ID NO:214).

In some embodiments, this invention provides an isolated polypeptide comprising an edge portion of C03950_{—}3_P9 (SEQ. ID NO:214), comprising an amino acid sequence being at least about 70%, optionally at least about 80%, preferably at least about 85%, more preferably at least about 90% and most preferably at least about 95% homologous to the sequence DVTS (SEQ. ID NO: 349) of C03950_{—}3_P9 (SEQ. ID NO:214).

In some embodiments, such isolated chimeric proteins or polypeptides comprise a first amino acid sequence being at least about 90% homologous to amino acids 1-810 of NP_{—}057062 (SEQ. ID NO:210), which also corresponds to amino acids 1-810 of C03950_{—}3_P9 (SEQ. ID NO:214), an second amino acid sequence being at least about 70%, optionally at least about 80%, preferably at least about 85%, more preferably at least about 90% and most preferably at least about 95% homologous to a polypeptide having the sequence DVTS (SEQ. ID NO: 349) corresponding to amino acids 811-814 of C03950_{—}3_P9 (SEQ. ID NO:214), wherein said first amino acid sequence and second amino acid sequence are contiguous and in a sequential order.

In some embodiments, such isolated chimeric proteins or polypeptides comprise a first amino acid sequence being at least about 90% homologous to amino acids 1-810 of Q9Y2V6_HUMAN (SEQ. ID NO:210), which also corresponds to amino acids 1-810 of C03950_{—}3_P9 (SEQ. ID

NO:214), and a second amino acid sequence being at least about 70%, optionally at least about 80%, preferably at least about 85%, more preferably at least about 90% and most preferably at least about 95% homologous to a polypeptide having the sequence DVTS (SEQ. ID NO: 349) corresponding to amino acids 811-814 of C03950_{—}3_P9 (SEQ. ID NO:214), wherein said first amino acid sequence and second amino acid sequence are contiguous and in a sequential order.

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MGNYKSRPTQTCT (SEQ. ID NO: 346) corresponding to amino acids 1-13 of C03950_{—}3_P9 (SEQ. ID NO:214), a second amino acid sequence being at least about 90% homologous to amino acids 132-367 of Q6MZS9_HUMAN (SEQ. ID NO:211), which also corresponds to amino acids 14-249 of C03950_{—}3_P9 (SEQ. ID NO:214), a bridging amino acid I corresponding to amino acid 250 of C03950_{—}3_P9 (SEQ. ID NO:214), a third amino acid sequence being at least about 90% homologous to amino acids 369-484 of Q6MZS9_HUMAN (SEQ. ID NO:211), which also corresponds to amino acids 251-366 of C03950_{—}3_P9 (SEQ. ID NO:214), bridging amino acid N corresponding to amino acid 367 of C03950_{—}3_P9 (SEQ. ID NO:214), a fourth amino acid sequence being at least about 90% homologous to amino acids 486-708 of Q6MZS9_HUMAN (SEQ. ID NO:211), which also corresponds to amino acids 368-590 of C039503_{—}3_P9 (SEQ. ID NO:214), and a fifth amino acid sequence being at least about 70%, optionally at least about 80%, preferably at least about 85%, more preferably at least about 90% and most preferably at least about 95% homologous to a polypeptide having the sequence SHNILLYEDGHAVVADFGESRFLQSLDEDNMTKQPGNLRWMAPEVFTQCTRYTIKADVFSYA LCLWELLTGEEPFAHLKPAAAAADMAYHEIERPPIGYSTKPISSLLIRGWNACPEGRPEFSEVVM KLEECLCNIELMSPASSNSSGSLSPSSSSDCLVNRGGPGRSHVAALRSRFELEYALNARSYAAL SQSAGQYSSQGLSLEEMKRSLQYTPIDKYDVTS (SEQ. ID NO: 350) corresponding to amino acids 591-814 of C03950_{—}3_P9 (SEQ. ID NO:214), wherein said first amino acid sequence, second amino acid sequence, bridging amino acid, third amino acid sequence, bridging amino kid, fourth amino acid sequence and fifth amino acid sequence are contiguous and in a sequential order.

In some embodiments, this invention provides an isolated polypeptide comprising a head of C03950_{—}3_P9 (SEQ. ID NO:214), comprising a polypeptide being at least about 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P9 (SEQ. ID NO:214).

In some embodiments, this invention provides an isolated polypeptide comprising an edge portion of C03950_{—}3_P9 (SEQ. ID NO:214), comprising an amino acid sequence being at least about 70%, optionally at least about 80%, preferably at least about 85%, more preferably at least about 90% and most preferably at least about 95% homologous to the sequence SHNILLYEDGHAVVADFGESRFLQSLDEDNMTKQPGNLRWMAPEVFTQCTRYTIKADVFSYA LCLWEELTGEEPFAHLKPAAAAADMAYHHERPPIGYSIPKPISSLLIRGWNACPEGRPEFSEVVM KLEECLCNIELMSPASSNSSGSLSPSSSSDCLVNRGGPGRSHVAALRSRFELEYALNARSYAAL SQSAGQYSSQGLSLEEMKRSLQYTPIDKYDVTS (SEQ. ID NO: 350) of C03950_{—}3_P9 (SEQ. ID NO:214).

In some embodiments, the isolated chimeric proteins or polypeptides of the invention comprise an amino acid sequence corresponding to or homologous to C03950_{—}3_P10 (SEQ. ID NO:215).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence PSPCGLHFLIPWLTQ (SEQ. ID NO: 351) corresponding to amino acids 1-15 of C03950_{—}3_P10 (SEQ. ID NO:215), and a second amino acid sequence being at least about 90% homologous to amino acids 213-936 of TNI3K_HUMAN (SEQ. ID NO: 396), which also corresponds to amino acids 16-739 of C03950_{—}3_P10 (SEQ. ID NO:215), wherein said first amino acid sequence and second amino acid sequence are contiguous and in a sequential order.

In some embodiments, this invention provides an isolated polypeptide comprising a head of C03950_{—}3_P10 (SEQ. ID NO:215), comprising a polypeptide being at least about 70%, optionally at least about 80%, preferably at least about 85%, more preferably at least about 90% and most preferably at least about 95% homologous to the sequence PSPCGLHFLIPWLTQ (SEQ. ID NO: 351) of C03950_{—}3_P10 (SEQ. ID NO:215).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence PSPCGLHFLIPWLTQ (SEQ. ID NO: 351) corresponding to amino acids 1-15 of C03950_{—}3_P10 (SEQ. ID NO:215), a second amino acid sequence being at least about 90% homologous to amino acids 230-367 of Q6MZS9_HUMAN (SEQ. ID NO:211), which also corresponds to amino acids 16-153 of C03950_{—}3_P10 (SEQ. ID NO:215), a bridging amino acid I corresponding to amino acid 154 of C03950_{—}3_P10 (SEQ. ID NO:215), a third amino acid sequence being at least about 90% homologous to amino acids 369-484 of Q6MZS9_HUMAN (SEQ. ID NO:211), which also corresponds to amino acids 155-270 of C03950_{—}3_P10 (SEQ. ID NO:215), a bridging amino acid N corresponding to amino acid 271 of C03950_{—}3_P10 (SEQ. ID NO:215), a fourth amino acid sequence being at least about 90% homologous to amino acids 486-708 of Q6MZS9_HUMAN (SEQ. ID NO:211), which also corresponds to amino acids 272-494 of C03950_{—}3_P10 (SEQ. ID NO:215), and a fifth amino acid sequence being at least about 70%, optionally at least about 80%, preferably at least about 85%, more preferably at least about 90% and most preferably at least about 95% homologous to a polypeptide having the sequence SHNILLYEDGHAVVADFGESRFLQSLDEDNMTKQPGNLRWMAPEVFTQCTRYTIKADVFSYA LCLWEILTGEIPFAHLICPAAAAADMAYHHIRPPIGYSIPKPIS SLLIRGWNACPEGRPEFSEVVIVI KLEECLCNIELMSPASSNSSGSLSPSSSSDCINNRGGPGRSHVAALRSRFELEYALNARSYAAL SQSAGQYSSQGLSLEEMKRSLQYTPIDKYGYVSDPMSSMHFHSCRNSS(SEQ. ID NO: 352) corresponding to amino acids 495-739 of C03950_{—}3_P10 (SEQ. ID NO:215), wherein said first amino acid sequence, second amino acid sequence, bridging amino acid, third amino acid sequence, bridging amino acid, fourth amino acid sequence and fifth amino acid sequence are contiguous and in a sequential order.

In some embodiments, this invention provides an isolated polypeptide comprising an edge portion of C03950_{—}3_P10 (SEQ. ID NO:215), comprising an amino acid sequence being at least about 70%, optionally at least about 80%, preferably at least about 85%, more preferably at least about 90% and most preferably at least about 95% homologous to the sequence—SHNILLYEDGHAVVADFGESRFLQSLDEDNMTKQPGNLRWMAPEVFTQCTRYTIKADVFSYA LCLWEILTGEIPFAHLKPAAAAADMAYHHIRPPIGYSIPKPISSLLIR.GWNACPEGRPEFSEWM KLEECLCNIELMSPASSNSSGSLSPSSSSDCLVNRGGPGRSHVAALRSRFELEYALNARSYAAL SQSAGQYSSQGLSLEEMIGISLQYTPIDKYGYVSDPMSSMBFHSCRNSS(SEQ. ID NO: 352) of C03950_{—}3_P10 (SEQ. ID NO:215).

In some embodiments, the isolated chimeric proteins or polypeptides of the invention comprise an amino acid sequence corresponding to or homologous to C03950_{—}3_P11 (SEQ. ID NO:216).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence PSPCGLHFLIPWLTQ (SEQ. ID NO: 351) corresponding to amino acids 1-15 of C03950_{—}3_P11 (SEQ. ID NO:216), a second amino acid sequence being at least about 90% homologous to amino acids 213-691 of TNI3K_HUMAN (SEQ. ID NO: 396), which also corresponds to amino acids 16-494 of C03950_{—}3_P11 (SEQ. ID NO:216), a third amino acid sequence being at least about 70%, optionally at least about 80%, preferably at least about 85%, more preferably at least about 90% and most preferably at least about 95%, homologous to a polypeptide having the sequence RSAITSRIWITHSICIWRGAHYFNREECNFRCMLTSAILK corresponding to amino acids 495-534 of C03950_{—}3_P11 (SEQ. ID NO:216), and a fourth amino acid sequence being at least about 90% homologous to amino acids 710-936 of TNI3K_HUMAN (SEQ. ID NO: 396), which also corresponds to amino acids 535-761 of C03950_{—}3_P11 (SEQ. ID NO:216), wherein said first amino acid sequence, second amino acid sequence, third amino acid sequence and fourth amino acid sequence are contiguous and in a sequential order.

In some embodiments, this invention provides an isolated polypeptide comprising a head of C03950_{—}3_P11 (SEQ. ID NO:216), comprising a polypeptide being at least about 70%, optionally at least about 80%, preferably at least about 85%, more preferably at least about 90% and most preferably at least about 95% homologous to the sequence PSPCGLHFLIPWLTQ (SEQ. ID NO: 351) of C03950_{—}3_P11 (SEQ. ID NO:216).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence PSPCGLHFLIPWLTQ (SEQ. ID NO: 351) corresponding to amino acids 1-15 of C03950_{—}3_P11 (SEQ. ID NO:216), a second amino acid sequence being at least about 90% homologous to amino acids 230-367 of Q6MZS9_HUMAN (SEQ. ID NO:211), which also corresponds to amino acids 16-153 of C03950_{—}3_P11 (SEQ. ID NO:216), a bridging amino acid I corresponding to amino acid 154 of C03950_{—}3_P11 (SEQ. ID NO:216), a third amino acid sequence being at least about 90% homologous to amino acids 369-484 of Q6MZS9_HUMAN (SEQ. ID NO:211), which also corresponds to amino acids 155-270 of C03950_{—}3_P11 (SEQ. ID NO:216), bridging amino acid N corresponding to amino acid 271 of C03950_{—}3_P11 (SEQ. ID NO:216), a fourth amino acid sequence being at least about 90% homologous to amino acids 486-709 of Q6MZS9_HUMAN (SEQ. ID NO:211), which also corresponds to amino acids 272-495 of C03950_{—}3_P11 (SEQ. ID NO:216), and a fifth amino acid sequence being at least about 70%, optionally at least about 80%, preferably at least about 85%, more preferably at least about 90% and most preferably at least about 95% homologous to a polypeptide having the sequence SAITSRIWITHSICIWRGAHYFNREECNFRCMLTSAILKESRFLQSLDEDNMTKQPGNLRWMA PEVFTQCTRYTIKADVFSYALCLWEILTGEIPFAHLKPAAAAADMAWIIIIRPPIGYSIPKPISSL LIRGWNACPEGRPEFSEVVMKLEECLCNIELMSPASSNSSGSLSPSSSSDCLVNRGGPGRSHVA ALRSRFELEYALNARSYAALSQSAGQYSSQGLSLEEMKRSLQYTPIDKYGYVSDPMSSMHFH SCRNSSSFEDSS (SEQ. ID NO: 345) corresponding to amino acids 496-761 of C03950_{—}3_P11 (SEQ. ID NO:216), wherein said first amino acid sequence, second amino acid sequence, bridging amino acid, third amino acid sequence, bridging amino acid, fourth amino acid sequence and fifth amino acid sequence are contiguous and in a sequential order.

In some embodiments, this invention provides an isolated polypeptide comprising a head of C03950_{—}3_P11 (SEQ. ID NO:216), comprising a polypeptide being at least about 70%, optionally at least about 80%, preferably at least about 85%, more preferably at least about 90% and most preferably at least about 95% homologous to the sequence PSPCGLITFLIPWLTQ (SEQ. ID NO: 351) of C03950_{—}3_P11 (SEQ. ID NO:216).

In some embodiments, this invention provides an isolated polypeptide comprising an edge portion of C03950_{—}3_P11 (SEQ. ID NO:216), comprising an amino acid sequence being at least about 70%, optionally at least about 80%, preferably at least about 85%, more preferably at least about 90% and most preferably at least about 95% homologous to the sequence—SAITSRIWITHSICIWRGAHYFNREECNFRCMLTSAILKESRFLQSLDEDNMTKQPGNLRWMA PEVFTQCTRYTIKADVFSYALCLWEILTGETPFAHLKPAAAAADMAYRFECRPPIGYSIPKPISSL LIRGWNACPEGRPEFSEVVMKLEECLCNIELMSPASSNSSGSLSPSSSSDCLVNRGGPGRSHVA ALRSRFELEYALNARSYAALSQSAGQYSSQGLSLEEMKRSLQYTPIDKYGYVSDPMSSMHPH SCRNSSSFEDSS (SEQ. ID NO: 345) of C03950_{—}3_P11 (SEQ. ID NO:216).

In some embodiments, the isolated chimeric proteins or polypeptides of the invention comprise an amino acid sequence corresponding to or homologous to C03950_{—}3_P12 (SEQ. ID NO:217). In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence AVRRGLREGGA (SEQ. ID NO: 342) corresponding to amino acids 1-11 of C03950_{—}3_P12 (SEQ. ID NO:217), a second amino acid sequence being at least about 90% homologous to amino acids 1-808 of TNI3K_HUMAN (SEQ. ID NO: 396), which also corresponds to amino acids 12-819 of C03950_{—}3_P12 (SEQ. ID NO:217), and a third amino acid sequence being at least about 70%, optionally at least about 80%, preferably at least about 85%, more preferably at least about 90% and most preferably at least about 95% homologous to a polypeptide having the sequence AKSRPSHYPVSSVYTETLKKKNEDRFGMWEEYLRR (SEQ. ID NO: 356) corresponding to amino acids 820-854 of C03950_{—}3_P12 (SEQ. ID NO:217), wherein said first amino acid sequence, second amino acid sequence and third amino acid sequence are contiguous and in a sequential order.

In some embodiments, this invention provides an isolated polypeptide comprising a head of C03950_{—}3_P12 (SEQ. ID NO:217), comprising a polypeptide being at least about 70%, optionally at least about 80%, preferably at least about 85%, more preferably at least about 90% and most preferably at least about 95% homologous to the sequence AVRRGLREGGA (SEQ. ID NO: 342) of C03950_{—}3_P12 (SEQ. ID NO:217).

In some embodiments, this invention provides an isolated polypeptide comprising an edge portion of C03950_{—}3_P12 (SEQ. ID NO:217), comprising an amino acid sequence being at least about 70%, optionally at least about 80%, preferably at least about 85%, more preferably at least about 90% and most preferably at least about 95% homologous to the sequence AKSRPSHYPVSSVYTETLKKICNEDRFGMWIEYLRR (SEQ. ID NO: 356) of C03950_{—}3_P12 (SEQ. ID NO:217).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence AVRRGLR (SEQ. ID NO: 344) corresponding to amino acids 1-7 of C03950_{—}3_P12 (SEQ. ID NO:217), a second amino acid sequence being at least about 90% homologous to amino acids 14-367 of Q6MZS9_HUMAN (SEQ. ID NO:211), which also corresponds to amino acids 8-361 of C03950_{—}3_P12 (SEQ. ID NO:217), a bridging amino acid I corresponding to amino acid 362 of C03950_{—}3_P12 (SEQ. ID NO:217), a third amino acid sequence being at least about 90% homologous to amino acids 369-484 of Q6MZS9_HUMAN (SEQ. ID NO:211), which also corresponds to amino acids 363-478 of C03950_{—}3_P12 (SEQ. ID NO:217), a bridging amino acid N corresponding to amino acid 479 of C03950_{—}3_P12 (SEQ. ID NO:217), a fourth amino acid sequence being at least about 90% homologous to amino acids 486-708 of Q6MZS9_HUMAN (SEQ. ID NO:211), which also corresponds to amino acids 480-702 of C03950_{—}3_P12 (SEQ. ID NO:217), and a fifth amino acid sequence being at least about 70%, optionally at least about 80%, preferably at least about 85%, more preferably at least about 90% and most preferably at least about 95% homologous to a polypeptide having the sequence SHNILLYEDGHAVVADFGESRPLQSLDEDNMTKQPGNLRWMAPEVFTQCTRYTIKADVFSYA LCLWEILTGELPFAHLKPAAAAADMAYHHIRPPIGYSIPKPISSLLIRGWNACPEAKSRPSHYPV SSVYTETLKKXNEDRFGMWIEYLRR (SEQ. ID NO: 358) corresponding to amino acids 703-854 of C03950_{—}3_P12 (SEQ. ID NO:217), wherein said first amino acid sequence, second amino acid sequence, bridging amino acid, third amino acid sequence, bridging amino acid, fourth amino acid sequence and fifth amino acid sequence are contiguous and in a sequential order.

In some embodiments, this invention provides an isolated polypeptide comprising a head of C03950_{—}3_P12 (SEQ. ID NO:217), comprising a polypeptide being at least about 70%, optionally at least about 80%, preferably at least about 85%, more preferably at least about 90% and most preferably at least about 95% homologous to the sequence AVRRGLR (SEQ. ID NO: 344) of C03950_{—}3_P12 (SEQ. ID NO:217).

In some embodiments, this invention provides an isolated polypeptide comprising an edge portion of C03950_{—}3_P12 (SEQ. ID NO:217), comprising an amino acid sequence being at least about 70%, optionally at least about 80%, preferably at least about 85%, more preferably at least about 90% and most preferably at least about 95% homologous to the sequence—SHNILLYEDGHAVVADFGESRFLQSLDEDNMTKQPGNLRWMAPEVFTQCTRYTIKADVFSYA LCLWEILTGEIPFAHLKPAAAAADMAYRHIRPPIGYSIPKPIS SLLIRGWNACPEAKSRPSHYPV SSVYTETLKKKNEDRFGMWIEYLRR (SEQ. ID NO: 358) of C03950_{—}3_P12 (SEQ. ID NO:217).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence—AVRRGLREGGAMAAARDPPEVSLREATQRICLRRFSELRGICLVARGEFWDIVAITAADEKQEL AYNQQLSEKLKRKELPLGVQYHVFVDPAGAKIGNGGSTLCALQCLEKLYGDKWNSFIILLITI S (SEQ. ID NO: 343) corresponding to amino acids 1-125 of C03950_{—}3_P12 (SEQ. ID NO:217), a second amino acid sequence being at least about 90% homologous to amino acids 14-707 of NP_{—}057062 (SEQ. ID NO:210), which also corresponds to amino acids 126-819 of C03950_{—}3_P12 (SEQ. ID NO:217), and a third amino acid sequence being at least about 70%, optionally at least about 80%, preferably at least about 85%, more preferably at least about 90% and most preferably at least about 95% homologous to a polypeptide having the sequence AKSRPSHYPVSSVYTETLKKKNEDRFGMWIEYLRR (SEQ. ID NO: 356) corresponding to amino acids 820-854 of C03950_{—}3_P12 (SEQ. ID NO:217), wherein said first amino acid sequence, second amino acid sequence and third amino acid sequence are contiguous and in a sequential order.

In some embodiments, this invention provides an isolated polypeptide comprising a head of C03950_{—}3_P12 (SEQ. ID NO:217), comprising a polypeptide being at least about 70%, optionally at least about 80%, preferably at least about 85%, more preferably at least about 90% and most preferably at least about 95% homologous to the sequence AVRRGLREGGAMAAARDPPEVSLREATQRKLRRFSELRGKINARGEFWDIVAITAADEKQEL AYNQQLSEKLICRICELPLGVQYHVFVDPAGAKIGNGGSTLCALQCLEICLYGDKWNSFTILLIH S (SEQ. ID NO: 343) of C03950_{—}3_P12 (SEQ. ID NO:217).

In some embodiments, the isolated chimeric proteins or polypeptides of the invention comprise an amino acid sequence corresponding to or homologous to In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MGNYKSRPTQTCT (SEQ. ID NO: 346) corresponding to amino acids 1-13 of C03950_{—}3_P13 (SEQ. ID NO:218), a second amino acid sequence being at least about 90% homologous to amino acids 115-808 of TNI3K_HUMAN (SEQ. ID NO: 396), which also corresponds to amino acids 14-707 of C03950_{—}3_P13 (SEQ. ID NO:218), and a third amino acid sequence being at least about 70%, optionally at least about 80%, preferably at least about 85%, more preferably at least about 90% and most preferably at least about 95% homologous to a polypeptide having the sequence AKSRPSHYPVSSVYTETLKKKNEDRFGMWIEYLRR (SEQ. ID NO: 356) corresponding to amino acids 708-742 of C03950_{—}3_P13 (SEQ. ID NO:218), wherein said first amino acid sequence, second amino acid sequence and third amino acid sequence are contiguous and in a sequential order.

In some embodiments, this invention provides an isolated polypeptide comprising a head of C03950_{—}3_P13 (SEQ. ID NO:218), comprising a polypeptide being at least about 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P13 (SEQ. ID NO:218).

In some embodiments, this invention provides an isolated polypeptide comprising an edge portion of C03950_{—}3_P13 (SEQ. ID NO:218), comprising an amino acid sequence being at least about 70%, optionally at least about 80%, preferably at least about 85%, more preferably at least about 90% and most preferably at least about 95% homologous to the sequence AKSRPSHYPVSSVYTETLKKKNEDREGMWEEYLRR (SEQ. ID NO: 356) of C03950_{—}3_P13 (SEQ. ID NO:218).

In some embodiments, such isolated chimeric proteins or polypeptides comprise a first amino acid sequence being at least about 90% homologous to amino acids 1-707 of NP_{—}057062 (SEQ. ID NO:210), which also corresponds to amino acids 1-707 of C03950_{—}3_P13 (SEQ. ID NO:218), and a second amino acid sequence being at least about 70%, optionally at least about 80%, preferably at least about 85%, more preferably at least about 90% and most preferably at least about 95% homologous to a polypeptide having the sequence AKSRPSHYPVSSVYTETLKKICNEDRFGMWIEYLRR (SEQ. ID NO: 356) corresponding to amino acids 708-742 of C03950_{—}3_P13 (SEQ. ID NO:218), wherein said first amino acid sequence and second amino acid sequence are contiguous and in a sequential order.

In some embodiments, such isolated chimeric proteins or polypeptides comprise a first amino acid sequence being at least about 70%, optionally at least about 80%, ₚreferably at least about 85%, more preferably at least about 90% and most preferably at least about 95%, homologous to a polypeptide having the sequence MGNYKSRPTQTCT (SEQ. ID NO: 346) corresponding to amino acids 1-13 of C03950_{—}3_P13 (SEQ. ID NO:218), a second amino acid sequence being at least about 90% homologous to amino acids 132-367 of Q6MZS9 HUMAN (SEQ. ID NO:211), which also corresponds to amino acids 14-249 of C03950_{—}3_P13 (SEQ. ID NO:218), a bridging amino acid I corresponding to amino acid 250 of C03950_{—}3_P13 (SEQ. ID NO:218), a third amino acid sequence being at least about 90% homologous to amino acids 369-484 of Q6MZS9_HUMAN (SEQ. ID NO:211), which also corresponds to amino acids 251-366 of C03950_{—}3P13 (SEQ. ID NO:218), a bridging amino acid N corresponding to amino acid 367 of C03950_{—}3_P13 (SEQ. ID NO:218), a fourth amino acid sequence being at least about 90% homologous to amino acids 486-708 of Q6MZS9_HUMAN (SEQ. ID NO:211), which also corresponds to amino acids 368-590 of C03950_{—}3_P13 (SEQ. ID NO:218), and a fifth amino acid sequence being at least about 70%, optionally at least about 80%, preferably at least about 85%, more preferably at least about 90% and most preferably at least about 95% homologous to a polypeptide having the sequence SHNILLYEDGHAVVADFGESRFLQSLDEDNMTKQPGNLRWMAPEVFTQCTRYTIKADVFSYA LCLWELLTGEIPFAHLKPAAAAADMAYHHIRPPIGYSIPKPISSLLIRGWNACPEAKSRPSHYPV SSVYTETLKKKNEDRFGMWIEYLRR (SEQ. ID NO: 358) corresponding to amino acids 591-742 of C03950_{—}3_P13 (SEQ. ID NO:218), wherein said first amino acid sequence, second amino acid sequence, bridging amino acid, third amino acid sequence, bridging amino acid, fourth amino acid sequence and fifth amino acid sequence are contiguous and in a sequential order.

In some embodiments, this invention provides an isolated polypeptide comprising an edge portion of C03950_{—}3_P13 (SEQ. ID NO:218), comprising an amino acid sequence being at least about 70%, optionally at least about 80%, preferably at least about 85%, more preferably at least about 90% and most preferably at least about 95% homologous to the sequence—SHNILLYEDGHAVVADFGESRFLQSLDEDNMTKQPGNLRWMAPEVFTQCTRYTIKADVFSYA LCLWElLTGEIPFAHLKPAAAAADMAYHRIRPPIGYSIPKPISSLLIRGWNACPEAKSRPSHYPV SSVYTETLKKKNEDRFGMWIEYLRR (SEQ. ID NO: 358) of C03950_{—}3_P13 (SEQ. ID NO:218).

In some embodiments, the isolated chimeric proteins or polypeptides of the invention comprise an amino acid sequence corresponding to or homologous to C03950_{—}3_P15 (SEQ. ID NO:219).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence AVRRGLREGGA (SEQ. ID NO: 342) corresponding to amino acids 1-11 of C03950_{—}3_P15 (SEQ. ID NO:219), a second amino acid sequence being at least about 90% homologous to amino acids 1-691 of TNI3K_HUMAN (SEQ. ID NO: 396), which also corresponds to amino acids 12-702 of C03950_{—}3_P15 (SEQ. ID NO:219), and a third amino acid sequence being at least about 70%, optionally at least about 80%, preferably at least about 85%, more preferably at least about 90% and most preferably at least about 95% homologous to a polypeptide having the sequence RYFFPK (SEQ. ID NO: 364) corresponding to amino acids 703-708 of C03950_{—}3_P15 (SEQ. ID NO:219), wherein said first amino acid sequence, second amino acid sequence and third amino acid sequence are contiguous and in a sequential order.

In some embodiments, this invention provides an isolated polypeptide comprising a head of C03950_{—}3_P15 (SEQ. ID NO:219), comprising a polypeptide being at least about 70%, optionally at least about 80%, preferably at least about 85%, more preferably at least about 90% and most preferably at least about 95% homologous to the sequence AVRRGLREGGA (SEQ. ID NO: 342) of C03950_{—}3_P15 (SEQ. ID NO:219).

In some embodiments, this invention provides an isolated polypeptide comprising an edge portion of C03950_{—}3_P15 (SEQ. ID NO:219), comprising an amino acid sequence being at least about 70%, optionally at least about 80%, preferably at least about 85%, more preferably at least about 90% and most preferably at least about 95% homologous to the sequence RYFFPK (SEQ. ID NO: 364) of C03950_{—}3_P15 (SEQ. ID NO:219).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence AVRRGLR (SEQ. ID NO: 344) corresponding to amino acids 1-7 of C03950_{—}3_P15 (SEQ. ID NO:219), a second amino acid sequence being at least about 90% homologous to amino acids 14-367 of Q6MZS9_HUMAN (SEQ. ID NO:211), which also corresponds to amino acids 8-361 of C03950_{—}3_P15 (SEQ. ID NO:219), a bridging amino acid I corresponding to amino acid 362 of C03950_{—}3_P15 (SEQ. ID NO:219), a third amino acid sequence being at least about 90% homologous to amino acids 369-484 of Q6MZS9_HUMAN (SEQ. ID NO:211), which also corresponds to amino acids 363-478 of C03950_{—}3_P15 (SEQ. ID NO:219), a bridging amino acid N corresponding to amino acid 479 of C03950_{—}3_P15 (SEQ. ID NO:219), and a fourth amino acid sequence being at least about 90% homologous to amino acids 486-714 of Q6MZS9_HUMAN (SEQ. ID NO:211), which also corresponds to amino acids 480-708 of C03950_{—}3_P15 (SEQ. ID NO:219), wherein said first amino acid sequence, second amino acid sequence, bridging amino acid, third amino acid sequence, bridging amino acid and fourth amino acid sequence are contiguous and in a sequential order.

In some embodiments, this invention provides an isolated polypeptide comprising a head of C03950_{—}3_P15 (SEQ. ID NO:219), comprising a polypeptide being at least about 70%, optionally at least about 80%, preferably at least about 85%, more preferably at least about 90% and most preferably at least about 95% homologous to the sequence AVRRGLR (SEQ. ID NO: 344) of C03950_{—}3_P15 (SEQ. ID NO:219).

A. An isolated chimeric polypeptide encoding for C03950_{—}3_P15 (SEQ. ID NO:219), comprising a first amino acid sequence being at least about 70%, optionally at least about 80%, preferably at least about 85%, more preferably at least about 90% and most preferably at least about 95%, homologous to a polypeptide having the sequence AVRRGLREGGAMAAARDPPEVSLREATQRICLRRFSELRGKINARGEFWDIVAITAADEKQEL AYNQQLSEKLKRKELPLGVQYHVFVDPAGAKIGNGGSTLCALQCLEKLYGDKWNSFTILLIII S (SEQ. ID NO: 343) corresponding to amino acids 1-125 of C03950_{—}3_P15 (SEQ. ID NO:219), second amino acid sequence being at least about 90% homologous to amino acids 14-590 of NP_{—}057062 (SEQ. ID NO:210), which also corresponds to amino acids 126-702 of C03950_{—}3_{—}1 (SEQ. ID NO:219), and a third amino acid sequence being at least about 70%, optionally at least about 80%, preferably at least about 85%, more preferably at least about 90% and most preferably at least about 95% homologous to a polypeptide having the sequence RYFFPK (SEQ. ID NO: 364) corresponding to amino acids 703-708 of C03950_{—}3_P15 (SEQ. ID NO:219), wherein said first amino acid sequence, second amino acid sequence and third amino acid sequence are contiguous and in a sequential order.

In some embodiments, this invention provides an isolated polypeptide comprising a head of C03950_{—}3_P15 (SEQ. ID NO:219), comprising a polypeptide being at least about 70%, optionally at least about 80%, preferably at least about 85%, more preferably at least about 90% and most preferably at least about 95% homologous to the sequence AVRRGLREGGAMAAARDPPEVSLREATQRKLRRFSELRGKLVARGEFWDNAITAADEKQEL AYNQQLSEKLKRKELPLGVQYHVFVDPAGAKIGNGGSTLCALQCLEKLYGDKWNSFTILLIE S (SEQ. ID NO: 343) of C03950_{—}3_P15 (SEQ. ID NO:219).

In some embodiments, the isolated chimeric proteins or polypeptides of the invention comprise an amino acid sequence corresponding to or homologous to C03950_{—}3_P17 (SEQ. ID NO:220).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MGNYKSRPTQTCT (SEQ. ID NO: 346) corresponding to amino acids 1-13 of C03950_{—}3_P17 (SEQ. ID NO:220), a second amino acid sequence being at least about 90% homologous to amino acids 115-691 of TNI3K_HUMAN (SEQ. ID NO: 396), which also corresponds to amino acids 14-590 of C03950_{—}3_P17 (SEQ. ID NO:220), and a third amino acid sequence being at least about 70%, optionally at least about 80%, preferably at least about 85%, more preferably at least about 90% and most preferably at least about 95% homologous to a polypeptide having the sequence RCCTGWLSCYHPD (SEQ. ID NO: 368) corresponding to amino acids 591-603 of C03950_{—}3_P17 (SEQ. ID NO:220), wherein said first amino acid sequence, second amino acid sequence and third amino acid sequence are contiguous and in a sequential order.

In some embodiments, this invention provides an isolated polypeptide comprising a head of C03950_{—}3_P17 (SEQ. ID NO:220), comprising a polypeptide being at least about 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P17 (SEQ. ID NO:220).

In some embodiments, this invention provides an isolated polypeptide comprising an edge portion of C03950_{—}3_P17 (SEQ. ID NO:220), comprising an amino acid sequence being at least about 70%, optionally at least about 80%, preferably at least about 85%, more preferably at least about 90% and most preferably at least about 95% homologous to the sequence RCCTGWLSCYHPD (SEQ. ID NO: 368) of C03950_{—}3_P17 (SEQ. ID NO:220).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MGNYKSRPTQTCT (SEQ. ID NO: 346) corresponding to amino acids 1-13 of C03950_{—}3_P17 (SEQ. ID NO:220), a second amino acid sequence being at least about 90% homologous to amino acids 132-367 of Q6MZS9_HUMAN (SEQ. ID NO:211), which also corresponds to amino acids 14-249 of C03950_{—}3_P17 (SEQ. ID NO:220), a bridging amino acid I corresponding to amino acid 250 of C03950_{—}3_P17 (SEQ. ID NO:220), a third amino acid sequence being at least about 90% homologous to amino acids 369-484 of Q6MZS9_HUMAN (SEQ. ID NO:211), which also corresponds to amino acids 251-366 of C03950_{—}3_P17 (SEQ. ID NO:220), a bridging amino acid N corresponding to amino acid 367 of C03950_{—}3_P17 (SEQ. ID NO:220), a fourth amino acid sequence being at least about 90% homologous to amino acids 486-709 of Q6MZS9_HUMAN (SEQ. ID NO:211), which also corresponds to amino acids 368-591 of C03950_{—}3_P17 (SEQ. ID NO:220), and a fifth amino acid sequence being at least about 70%, optionally at least about 80%, preferably at least about 85%, more preferably at least about 90% and most preferably at least about 95% homologous to a polypeptide having the sequence CCTGWLSCYHPD (SEQ. ID NO: 369) corresponding to amino acids 592-603 of C03950_{—}3_P17 (SEQ. ID NO:220), wherein said first amino acid sequence, second amino acid sequence, bridging amino acid, third amino acid sequence, bridging amino acid, fourth amino acid sequence and fifth amino acid sequence are contiguous and in a sequential order.

In some embodiments, this invention provides an isolated polypeptide comprising an edge portion of C03950_{—}3_P17 (SEQ. ID NO:220), comprising an amino acid sequence being at least about 70%, optionally at least about 80%, preferably at least about 85%, more preferably at least about 90% and most preferably at least about 95% homologous to the sequence CCTGWLSCYHPD (SEQ. ID NO: 369) of C03950_{—}3_P17 (SEQ. ID NO:220).

In some embodiments, such isolated chimeric proteins or polypeptides comprise a first amino acid sequence being at least about 90% homologous to amino acids 1-590 of NP_{—}057062 (SEQ. ID NO:210), which also corresponds to amino acids 1-590 of C03950_{—}3_P17 (SEQ. ID NO:220) second amino acid sequence being at least about 70%, optionally at least about 80%, preferably at least about 85%, more preferably at least about 90% and most preferably at least about 95% homologous to a polypeptide having the sequence RCCTGWLSCYHPD (SEQ. ID NO: 368) corresponding to amino acids 591-603 of C03950_{—}3_P17 (SEQ. ID NO:220), wherein said first amino acid sequence and second amino acid sequence are contiguous and in a sequential order.

In some embodiments, the isolated chimeric proteins or polypeptides of the invention comprise an amino acid sequence corresponding to or homologous to C03950_{—}3_P19 (SEQ. ID NO:221).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MGNYKSRPTQTCT (SEQ. ID NO: 346) corresponding to amino acids 1-13 of C03950_{—}3_P19 (SEQ. ID NO:221), a second amino acid sequence being at least about 90% homologous to amino acids 115-691 of TNI3K_HU1VIAN (SEQ. ID NO: 396), which also corresponds to amino acids 14-590 of C03950_{—}3_P19 (SEQ. ID NO:221), and a third amino acid sequence being at least about 70%, optionally at least about 80%, preferably at least about 85%, more preferably at least about 90% and most preferably at least about 95% homologous to a polypeptide having the sequence RAS (SEQ. ID NO: 370) corresponding to amino acids 591-593 of C03950_{—}3_P19 (SEQ. ID NO:221), wherein said first amino acid sequence, second amino acid sequence and third amino acid sequence are contiguous and in a sequential order.

In some embodiments, this invention provides an isolated polypeptide comprising an edge portion of C03950_{—}3_P19 (SEQ. ID NO:221), comprising an amino acid sequence being at least about 70%, optionally at least about 80%, preferably at least about 85%, more preferably at least about 90% and most preferably at least about 95% homologous to the sequence RAS (SEQ. ID NO: 370) of C03950_{—}3_P19 (SEQ. ID NO:221).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MGNYKSRPTQTCT (SEQ. ID NO: 346) corresponding to amino acids 1-13 of C03950_{—}3_P19 (SEQ. ID NO:221), a second amino acid sequence being at least about 90% homologous to amino acids 132-367 of Q6MZS9_HUMAN (SEQ. ID NO:211), which also corresponds to amino acids 14-249 of C03950_{—}3_P19 (SEQ. ID NO:221), a bridging amino acid I corresponding to amino acid 250 of C03950_{—}3_P19 (SEQ. ID NO:221), a third amino acid sequence being at least about 90% homologous to amino acids 369-484 of Q6MZS9_HUMAN (SEQ. ID NO:211), which also corresponds to amino acids 251-366 of C03950_{—}3_P19 (SEQ. ID NO:221), bridging amino acid N corresponding to amino acid 367 of C03950_{—}3_P19 (SEQ. ID NO:221), a fourth amino acid sequence being at least about 90% homologous to amino acids 486-709 of Q6MZS9_HUMAN (SEQ. ID NO:211), which also corresponds to amino acids 368-591 of C03950_{—}3_P19 (SEQ. ID NO:221), and a fifth amino acid sequence being at least about 70%, optionally at least about 80%, preferably at least about 85%, more preferably at least about 90% and most preferably at least about 95% homologous to a polypeptide having the sequence AS corresponding to amino acids 592-593 of C03950_{—}3_P19 (SEQ. ID NO:221), wherein said first amino acid sequence, second amino acid sequence, bridging amino acid, third amino acid sequence, bridging amino acid, fourth amino acid sequence and fifth amino acid sequence are contiguous and in a sequential order.

In some embodiments, such isolated chimeric proteins or polypeptides comprise a first amino acid sequence being at least about 90% homologous to amino acids 1-590 of NP_{—}057062 (SEQ. ID

NO:210), which also corresponds to amino acids 1-590 of C03950_{—}3_P19 (SEQ. ID NO:221) second amino acid sequence being at least about 70%, optionally at least about 80%, preferably at least about 85%, more preferably at least about 90% and most preferably at least about 95% homologous to a polypeptide having the sequence RAS (SEQ. ID NO: 370) corresponding to amino acids 591-593 of C03950_{—}3_P19 (SEQ. ID NO:221), wherein said first amino acid sequence and second amino acid sequence are contiguous and in a sequential order.

In some embodiments, the isolated chimeric proteins or polypeptides of the invention comprise an amino acid sequence corresponding to or homologous to C03950_{—}3_P20 (SEQ. ID NO:222).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence AVRRGLREGGA (SEQ. ID NO: 342) corresponding to amino acids 1-11 of C03950_{—}3_P20 (SEQ. ID NO:222), a second amino acid sequence being at least about 90% homologous to amino acids 1-657 of TNI3K_HUMAN (SEQ. ID NO: 396), which also corresponds to amino acids 12-668 of C03950_{—}3_P20 (SEQ. ID NO:222), and a third amino acid sequence being at least about 70%, optionally at least about 80%, preferably at least about 85%, more preferably at least about 90% and most preferably at least about 95% homologous to a polypeptide having the sequence YGSFVLIYPWTFRRNYSCNTSEGFPLDEPSPFEI (SEQ. ID NO: 372) corresponding to amino acids 669-702 of C03950_{—}3_P20 (SEQ. ID NO:222), wherein said first amino acid sequence, second amino acid sequence and third amino acid sequence are contiguous and in a sequential order.

In some embodiments, this invention provides an isolated polypeptide comprising a head of C03950_{—}3_P20 (SEQ. ID NO:222), comprising a polypeptide being at least about 70%, optionally at least about 80%, preferably at least about 85%, more preferably at least about 90% and most preferably at least about 95% homologous to the sequence AVRRGLREGGA (SEQ. ID NO: 342) of C03950_{—}3_P20 (SEQ. ID NO:222).

In some embodiments, this invention provides an isolated polypeptide comprising an edge portion of C03950_{—}3_P20 (SEQ. ID NO:222), comprising an amino acid sequence being at least about 70%, optionally at least about 80%, preferably at least about 85%, more preferably at least about 90% and most preferably at least about 95% homologous to the sequence YGSFVUYPWTFRRNYSCNTSEGFPLDEPSPFEI (SEQ. ID NO: 372) of C03950_{—}3_P20 (SEQ. ID NO:222).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence AVRRGLR (SEQ. ID NO: 344) corresponding to amino acids 1-7 of C03950_{—}3_P20 (SEQ. ID NO:222), a second amino acid sequence being at least about 90% homologous to amino acids 14-367 of Q6MZS9_HUMAN (SEQ. ID NO:211), which also corresponds to amino acids 8-361 of C03950_{—}3_P20 (SEQ. ID NO:222), a bridging amino acid I corresponding to amino acid 362 of C03950_{—}3_P20 (SEQ. ID NO:222), a third amino acid sequence being at least about 90% homologous to amino acids 369-484 of Q6MZS9_HUMAN (SEQ. ID NO:211), which also corresponds to amino acids 363-478 of C03950_{—}3_P20 (SEQ. ID NO:222), a bridging amino acid N corresponding to amino acid 479 of C03950_{—}3_P20 (SEQ. ID NO:222), a fourth amino acid sequence being at least about 90% homologous to amino acids 486-674 of Q6MZS9_HUMAN (SEQ. ID NO:211), which also corresponds to amino acids 480-668 of C03950_{—}3_P20 (SEQ. ID NO:222), and a fifth amino acid sequence being at least about 70%, optionally at least about 80%, preferably at least about 85%, more preferably at least about 90% and most preferably at least about 95% homologous to a polypeptide having the sequence YGSFVLIYPWTFRRNYSCNTSEGFPLDEPSPFEI (SEQ. ID NO: 372) corresponding to amino acids 669-702 of C03950_{—}3_P20 (SEQ. ID NO:222), wherein said first amino acid sequence, second amino acid sequence, bridging amino acid, third amino acid sequence, bridging amino acid, fourth amino acid sequence and fifth amino acid sequence are contiguous and in a sequential order.

In some embodiments, this invention provides an isolated polypeptide comprising a head of C03950_{—}3_P20 (SEQ. ID NO:222), comprising a polypeptide being at least about 70%, optionally at least about 80%, preferably at least about 85%, more preferably at least about 90% and most preferably at least about 95% homologous to the sequence AVRRGLR (SEQ. ID NO: 344) of C03950_{—}3_P20 (SEQ. ID NO:222).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence—AVRRGLREGGAMAAARDPPEVSLREATQRICIARFSELRGKLVARGEFWDIVAITAADEKQEL AYNQQLSEKLICRICELPLGVQYHVFVDPAGAKIGNGGSTLCALQCLEKLYGDKWNSFTILLIH S (SEQ. ID NO: 343) corresponding to amino acids 1-125 of C03950_{—}3_P20 (SEQ. ID NO:222), a second amino acid sequence being at least about 90% homologous to amino acids 14-556 of NP_{—}057062 (SEQ. ID NO:210), which also corresponds to amino acids 126-668 of C03950_{—}3_P20 (SEQ. ID NO:222), and a third amino acid sequence being at least about 70%, optionally at least about 80%, preferably at least about 85%, more preferably at least about 90% and most preferably at least about 95% homologous to a polypeptide having the sequence YGSFVLIYPWTFRRNYSCNTSEGFPLDEPSPFEI (SEQ. ID NO: 372) corresponding to amino acids 669-702 of C03950_{—}3_P20 (SEQ. ID NO:222), wherein said first amino acid sequence, second amino acid sequence and third amino acid sequence are contiguous and in a sequential order.

In some embodiments, this invention provides an isolated polypeptide comprising a head of C03950_{—}3_P20 (SEQ. ID NO:222), comprising a polypeptide being at least about 70%, optionally at least about 80%, preferably at least about 85%, more preferably at least about 90% and most preferably at least about 95% homologous to the sequence—AVRRGLREGGAMAAARDPPEVSLREATQRICLRRFSELRGKLVARGEFWDIVAITAADEKQEL AYNQQLSEKLKRKELPLGVQYHVFVDPAGAKIGNGGSTLCALQCLEKLYGDKWNSFTILLIH S (SEQ. ID NO: 343) of C03950_{—}3_P20 (SEQ. ID NO:222).

In some embodiments, the isolated chimeric proteins or polypeptides of the invention comprise an amino acid sequence corresponding to or homologous to C03950_{—}3_P21 (SEQ. ID NO:223). In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MGNYKSRPTQTCT (SEQ. ID NO: 346) corresponding to amino acids 1-13 of C03950_{—}3_P21 (SEQ. ID NO:223), a second amino acid sequence being at least about 90% homologous to amino acids 115-657 of TNI3K_HUMAN (SEQ. ID NO: 396), which also corresponds to amino acids 14-556 of C03950_{—}3_P21 (SEQ. ID NO:223), and a third amino acid sequence being at least about 70%, optionally at least about 80%, preferably at least about 85%, more preferably at least about 90% and most preferably at least about 95% homologous to a polypeptide having the sequence YGSFVLIYPWTFRRNYSCNTSEGFPLDEPSPFEI (SEQ. ID NO: 372) corresponding to amino acids 557-590 of C03950_{—}3_P21 (SEQ. ID NO:223), wherein said first amino acid sequence, second amino acid sequence and third amino acid sequence are contiguous and in a sequential order.

In some embodiments, this invention provides an isolated polypeptide comprising a head of C03950_{—}3_P21 (SEQ. ID NO:223), comprising a polypeptide being at least about 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P21 (SEQ. ID NO:223).

In some embodiments, this invention provides an isolated polypeptide comprising an edge portion of C03950_{—}3_P21 (SEQ. ID NO:223), comprising an amino acid sequence being at least about 70%, optionally at least about 80%, preferably at least about 85%, more preferably at least about 90% and most preferably at least about 95% homologous to the sequence YGSFVLIYPWTFRRNYSCNTSEGFPLDEPSPFEI (SEQ. ID NO: 372) of C03950_{—}3_P21 (SEQ. ID NO:223).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MGNYKSRPTQTCT (SEQ. ID NO: 346) corresponding to amino acids 1-13 of C03950_{—}3_P21 (SEQ. ID NO:223), a second amino acid sequence being at least about 90% homologous to amino acids 132-367 of Q6MZS9_HUMAN (SEQ. ID NO:211), which also corresponds to amino acids 14-249 of C03950_{—}3_P21 (SEQ. ID NO:223), a bridging amino acid I corresponding to amino acid 250 of C03950_{—}3_P21 (SEQ. ID NO:223), a third amino acid sequence being at least about 90% homologous to amino acids 369-484 of Q6MZS9_HUMAN (SEQ. ID NO:211), which also corresponds to amino acids 251-366 of C03950_{—}3_P21 (SEQ. ID NO:223), a bridging amino acid N corresponding to amino acid 367 of C03950_{—}3_P21 (SEQ. ID NO:223), a fourth amino acid sequence being at least about 90% homologous to amino acids 486-674 of Q6MZS9_HUMAN (SEQ. ID NO:211), which also corresponds to amino acids 368-556 of C03950_{—}3_P21 (SEQ. ID NO:223), and a fifth amino acid sequence being at least about 70%, optionally at least about 80%, preferably at least about 85%, more preferably at least about 90% and most preferably at least about 95% homologous to a polypeptide having the sequence YGSFVLIYPWTFRRNYSCNTSEGFPLDEPSPFEI (SEQ. ID NO: 372) corresponding to amino acids 557-590 of C03950_{—}3_P21 (SEQ. ID NO:223), wherein said first amino acid sequence, second amino acid sequence, bridging amino acid, third amino acid sequence, bridging amino acid, fourth amino acid sequence and fifth amino acid sequence are contiguous and in a sequential order.

In some embodiments, such isolated chimeric proteins or polypeptides comprise a first amino acid sequence being at least about 90% homologous to amino acids 1-556 of NP_{—}057062 (SEQ. ID NO:210), which also corresponds to amino acids 1-556 of C03950_{—}3_P21 (SEQ. ID NO:223), a second amino acid sequence being at least about 70%, optionally at least about 80%, preferably at least about 85%, more preferably at least about 90% and most preferably at least about 95% homologous to a polypeptide having the sequence YGSFVLIYPWTFRRNYSCNTSEGFPLDEPSPFEI (SEQ. ID NO: 372) corresponding to amino acids 557-590 of C03950_{—}3_P21 (SEQ. ID NO:223), wherein said first amino acid sequence and second amino acid sequence are contiguous and in a sequential order.

In some embodiments, the isolated chimeric proteins or polypeptides of the invention comprise an amino acid sequence corresponding to or homologous to C03950_{—}3_P23 (SEQ. ID NO:224).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MGNYKSRPTQTCT (SEQ. ID NO: 346) corresponding to amino acids 1-13 of C03950_{—}3_P23 (SEQ. ID NO:224), a second amino acid sequence being at least about 90% homologous to amino acids 132-367 of Q6MZS9_HUMAN (SEQ. ID NO:211), which also corresponds to amino acids 14-249 of C03950_{—}3_P23 (SEQ. ID NO:224), a bridging amino acid I corresponding to amino acid 250 of C03950_{—}3_P23 (SEQ. ID NO:224), a third amino acid sequence being at least about 90% homologous to amino acids 369-484 of Q6MZS9_HUMAN (SEQ. ID NO:211), which also corresponds to amino acids 251-366 of C03950_{—}3_P23 (SEQ. ID NO:224), a bridging amino acid N corresponding to amino acid 367 of C03950_{—}3_P23 (SEQ. ID NO:224), a fourth amino acid sequence being at least about 90% homologous to amino acids 486-590 of Q6MZS9_HUMAN (SEQ. ID NO:211), which also corresponds to amino acids 368-472 of C03950_{—}3_P23 (SEQ. ID NO:224), and a fifth amino acid sequence being at least about 70%, optionally at least about 80%, preferably at least about 85%, more preferably at least about 90% and most preferably at least about 95% homologous to a polypeptide having the sequence NLK (SEQ. ID NO: 378) corresponding to amino acids 473-475 of C03950_{—}3_P23 (SEQ. ID NO:224), wherein first amino acid sequence, second amino acid sequence, bridging amino acid, third amino acid sequence, bridging amino acid, fourth amino acid sequence and fifth amino acid sequence are contiguous and in a sequential order.

In some embodiments, this invention provides an isolated polypeptide comprising a head of C03950_{—}3_P23 (SEQ. ID NO:224), comprising a polypeptide being at least about 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P23 (SEQ. ID NO:224).

In some embodiments, this invention provides an isolated polypeptide comprising an edge portion of C03950_{—}3_P23 (SEQ. ID NO:224), comprising an amino acid sequence being at least about 70%, optionally at least about 80%, preferably at least about 85%, more preferably at least about 90% and most preferably at least about 95% homologous to the sequence NLK (SEQ. ID NO: 378) of C03950_{—}3_P23 (SEQ. ID NO:224).

In some embodiments, such isolated chimeric proteins or polypeptides comprise a first amino acid sequence being at least about 90% homologous to amino acids 1-472 of NP_{—}057062 (SEQ. ID NO:210), which also corresponds to amino acids 1-472 of C03950_{—}3_P23 (SEQ. ID NO:224), a second amino acid sequence being at least about 70%, optionally at least about 80%, preferably at least about 85%, more preferably at least about 90% and most preferably at least about 95% homologous to a polypeptide having the sequence NLK (SEQ. ID NO: 378) corresponding to amino acids 473-475 of C03950_{—}3_P23 (SEQ. ID NO:224), wherein said first amino acid sequence and second amino acid sequence are contiguous and in a sequential order.

In some embodiments, the isolated chimeric proteins or polypeptides of the invention comprise an amino acid sequence corresponding to or homologous to C03950_{—}3_P28 (SEQ. ID NO:227).

In some embodiments, such isolated chimeric proteins or polypeptides comprise a first amino acid sequence being at least about 90% homologous to amino acids 1-691 of TNI3K_HUMAN (SEQ. ID NO: 396), which also corresponds to amino acids 1-691 of C03950_{—}3_P28 (SEQ. ID NO:227), a second amino acid sequence being at least about 70%, optionally at least about 80%, preferably at least about 85%, more preferably at least about 90% and most preferably at least about 95%, homologous to a pOlypeptide having the sequence RSAITSRIWITHSICIWRGAHYFNREECNFRCMLTSAILK corresponding to amino acids 692-731 of C03950_{—}3_P28 (SEQ. ID NO:227), and a third amino acid sequence being at least about 90% homologous to amino acids 710-936 of TNI3K_HUMAN (SEQ. ID NO: 396), which also corresponds to amino acids 732-958 of C03950_{—}3_P28 (SEQ. ID NO:227), wherein said first amino acid sequence, second amino acid sequence and third amino acid sequence are contiguous and in a sequential order.

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence—MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHS (SEQ. ID NO: 379) corresponding to amino acids 1-114 of C03950_{—}3_P28 (SEQ. ID NO:227), a second amino acid sequence being at least about 90% homologous to amino acids 14-590 of NP_{—}057062 (SEQ. ID NO:210), which also corresponds to amino acids 115-691 of C03950_{—}3_P28 (SEQ. ID NO:227), third amino acid sequence being at least about 70%, optionally at least about 80%, preferably at least about 85%, more preferably at least about 90% and most preferably at least about 95%, homologous to a polypeptide having the sequence RSAITSRIWITHSICIWRGAHYFNREECNFRCMLTSAILK corresponding to amino acids 692-731 of C03950_{—}3_P28 (SEQ. ID NO:227), and a fourth amino acid sequence being at least about 90% homologous to amino acids 609-835 of NP_{—}057062 (SEQ. ID NO:210), which also corresponds to amino acids 732-958 of C03950_{—}3_P28 (SEQ. ID NO:227), wherein said first amino acid sequence, second amino acid sequence, third amino acid sequence and fourth amino acid sequence are contiguous and in a sequential order.

In some embodiments, this invention provides an isolated polypeptide comprising a head of C03950_{—}3_P28 (SEQ. ID NO:227), comprising a polypeptide being at least about 70%, optionally at least about 80%, preferably at least about 85%, more preferably at least about 90% and most preferably at least about 95% homologous to the sequence MAAARDPPEVSLREATQRICLRRFSELRGICLVARGEFWDIVAITAADEKQELAYNQQLSEICLK RKELPLGVQYHVEVDPAGAKIGNGGSTLCALQCLEKLYGDKWNSFTILLIHS (SEQ. ID NO: 379) of C03950_{—}3_P28 (SEQ. ID NO:227).

In some embodiments, such isolated chimeric proteins or polypeptides comprise a first amino acid sequence being at least about 70%, optionally at least about 80%, preferably at least about 85%, more preferably at least about 90% and most preferably at least about 95%, hₒmologous to a polypeptide having the sequence MAAARDPPEVSLREATQRICLRRFSELRGKLVARGEFWDIVAITAADEKQELAYNQQLSEICLK RKELPLGVQYHVFVDPAGAKIGNGGSTLCALQCLEKLYGDKWNSFTILLIHS (SEQ. ID NO: 379) corresponding to amino acids 1-114 of C03950_{—}3_P28 (SEQ. ID NO:227), a second amino acid sequence being at least about 90% homologous to amino acids 14-590 of Q9Y2V6_HUMAN (SEQ. ID NO:210), which also corresponds to amino acids 115-691 of C03950_{—}3_P28 (SEQ. ID NO:227), a third amino acid sequence being at least about 70%, optionally at least about 80%, preferably at least about 85%, more preferably at least about 90% and most preferably at least about 95%, homologous to a polypeptide having the sequence RSAITSRIWITHSICIWRGAHYFNREECNERCMLTSAILK corresponding to amino acids 692-731 of C03950_{—}3_P28 (SEQ. ID NO:227), and a fourth amino acid sequence being at least about 90% homologous to amino acids 609-835 of Q9Y2V6_HUMAN (SEQ. ID NO:210), which also corresponds to amino acids 732-958 of C039503_P28 (SEQ. ID NO:227), wherein said first amino acid sequence, second amino acid sequence, third amino acid sequence and fourth amino acid sequence are contiguous and in a sequential order.

In some embodiments, such isolated chimeric proteins or polypeptides comprise a first amino acid sequence being at least about 90% homologous to amino acids 18-367 of Q6MZS9_HUMAN (SEQ. ID NO:211), which also corresponds to amino acids 1-350 of C03950_{—}3_P28 (SEQ. ID NO:227), a bridging amino acid I corresponding to amino acid 351 of C03950_{—}3_P28 (SEQ. ID NO:227), a second amino acid sequence being at least about 90% homologous to amino acids 369-484 of Q6MZS9_HUMAN (SEQ. ID NO:211), which also corresponds to amino acids 352-467 of C03950_{—}3_P28 (SEQ. ID NO:227), a bridging amino acid N corresponding to amino acid 468 of C03950_{—}3_P28 (SEQ. ID NO:227), a third amino acid sequence being at least about 90% homologous to amino acids 486-709 of Q6MZS9_HUMAN (SEQ. ID NO:211), which also corresponds to amino acids 469-692 of C03950_{—}3_P28 (SEQ. ID NO:227), and a fourth amino acid sequence being at least about 70%, optionally at least about 80%, preferably at least about 85%, more preferably at least about 90% and most preferably at least about 95% homologous to a polypeptide having the sequence SAITSRIWITHSICIWRGAHYFNREECNERCMLTSAILKESRFLQSLDEDNMTKQPGNLRWMA PEVFTQCTRYTIKADVFSYALCIAVEILTGEIPFAHLICPAAAAADMAYHHIRPPIGYSIPKPISSL LERGWNACPEGRPEFSEVVMICLEECLCNIELMSPASSNSSGSLSPSSSSDCLVNRGGPGRSHVA ALRSRFELEYALNARSYAALSQSAGQYSSQGLSLEEMKRSLQYTPEDKYGYVSDPMSSMHFH SCRNSSSFEDSS (SEQ. ID NO: 345) corresponding to amino acids 693-958 of C03950_{—}3_P28 (SEQ. ID NO:227), wherein said, first amino acid sequence, bridging amino acid, second amino acid sequence, bridging amino acid, third amino acid sequence and fourth amino acid sequence are contiguous and in a sequential order.

In some embodiments, this invention provides an isolated polypeptide comprising an edge portion of C03950_{—}3_P28 (SEQ. ID NO:227), comprising an amino acid sequence being at least about 70%, optionally at least about 80%, preferably at least about 85%, more preferably at least about 90% and most preferably at least about 95% homologous to the sequence—SAITSRIWITHSICIWRGAHYFNREECNFRCMLTSAILKESRFLQSLDEDNMTKQPGNLRWMA PEVFTQCTRYTIKADVFSYALCLWEILTGELPFAHLKPAAAAADMAYHHIRPPIGYSIPKPISSL LIRGWNACPEGRPEFSEVVMKLEECLCNIELMSPASSNSSGSLSPSSSSDCLVNRGGPGRSHVA ALRSRFELEYALNARSYAALSQSAGQYSSQGLSLEEMKRSLQYTPIDKYGYVSDPMSSMHPH SCRNSSSFEDSS (SEQ. ID NO: 345) of C03950_{—}3_P28 (SEQ. ID NO:227).

In some embodiments, such isolated chimeric proteins or polypeptides comprise a first amino acid sequence being at least about 90% homologous to amino acids 1-808 of TNI3K_HUMAN (SEQ. ID NO: 396), which also corresponds to amino acids 1-808 of C03950_{—}3_P31 (SEQ. ID NO:228), and a second amino acid sequence being at least about 70%, optionally at least about 80%, preferably at least about 85%, more preferably at least about 90% and most preferably at least about 95% homologous to a polypeptide having the sequence AKSRPSHYPVSSVYTETLKICKNEDRFGMWIEYLRR (SEQ. ID NO: 356) corresponding to amino acids 809-843 of C03950_{—}3_P31 (SEQ. ID NO:228), wherein said first amino acid sequence and second amino acid sequence are contiguous and in a sequential order.

In some embodiments, this invention provides an isolated polypeptide comprising an edge portion of C03950_{—}3_P31 (SEQ. ID NO:228), comprising an amino acid sequence being at least about 70%, optionally at least about 80%, preferably at least about 85%, more preferably at least about 90% and most preferably at least about 95% homologous to the sequence AKSRPSHYPVSSVYTETLKKKNEDRFGMWTEYLRR (SEQ. ID NO: 356) of C03950_{—}3_P31 (SEQ. ID NO:228).

In some embodiments, such isolated chimeric proteins or polypeptides comprise a first amino acid sequence being at least about 90% homologous to amino acids 18-367 of Q6MZS9_HUMAN (SEQ. ID NO:211), which also corresponds to amino acids 1-350 of C03950_{—}3_P31 (SEQ. ID NO:228), a bridging amino acid I corresponding to amino acid 351 of C03950_{—}3_P_P31 (SEQ. ID NO:228), a second amino acid sequence being at least about 90% homologous to amino acids 369-484 of Q6MZS9_HUMAN (SEQ. ID NO:211), which also corresponds to amino acids 352-467 of C03950_{—}3_P31 (SEQ. ID NO:228), a bridging amino acid N corresponding to amino acid 468 of C03950_{—}3_P31 (SEQ. ID NO:228), a third amino acid sequence being at least about 90% homologous to amino acids 486-708 of Q6MZS9_HUMAN (SEQ. ID NO:211), which also corresponds to amino acids 469-691 of C03950_{—}3_P31 (SEQ. ID NO:228), and a fourth amino acid sequence being at least about 70%, optionally at least about 80%, preferably at least about 85%, more preferably at least about 90% and most preferably at least about 95% homologous to a polypeptide having the sequence SHNILLYEDGHAVVADFGESRFLQSLDEDNMTKQPGNLRWMAPEVFTQCTRYTIKADVFSYA LCLWEILTGEIPFAHLKPAAAAADMAYHITIRPPIGYSIPKPISSLLIRGWNACPEAKSRPSHYPV SSVYTETLKKKNEDRFGMWIEYLRR (SEQ. ID NO: 358) corresponding to amino acids 692-843 of C03950_{—}3_P31 (SEQ. ID NO:228), wherein said, first amino acid sequence, bridging amino acid, second amino acid sequence, bridging amino acid, third amino acid sequence and fourth amino acid sequence are contiguous and in a sequential order.

In some embodiments, this invention provides an isolated polypeptide comprising an edge portion of C03950_{—}3_P31 (SEQ. ID NO:228), comprising an amino acid sequence being at least about 70%, optionally at least about 80%, preferably at least about 85%, more preferably at least about 90% and most preferably at least about 95% homologous to the sequence—SHNILLYEDGHAVVADFGESRFLQSLDEDNMTICQPGNLRWMAPEVFTQCTRYTIKADVESYA LCLWEILTGEIPFAHLKPAAAAADMAYHHIRPPIGYSIPKPIS SLLIRGWNACPEAKSRPSHYPV SSVYTETLICKKNEDREGMWIEYLRR (SEQ. ID NO: 358) of C03950_{—}3_P31 (SEQ. ID NO:228).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence—MAAARDPPEVSLREATQRKLRRESELRGKLVARGEFWDIVAITAADEKQELAYNQQLSEKLK RICELPLGVQYHVFVDPAGAKIGNGGSTLCALQCLEKLYGDKWNSFTILLTEIS (SEQ. ID NO: 379) corresponding to amino acids 1-114 of C03950_{—}3_P31 (SEQ. ID NO:228), a second amino acid sequence being at least about 90% homologous to amino acids 14-707 of NP_{—}057062 (SEQ. ID NO:210), which also corresponds to amino acids 115-808 of C03950_{—}3_P31 (SEQ. ID NO:228), and a third amino acid sequence being at least about 70%, optionally at least about 80%, preferably at least about 85%, more preferably at least about 90% and most preferably at least about 95% homologous to a polypeptide having the sequence AKSRPSHYPVSSVYTETLKICKNEDRFGMWIEYLRR (SEQ. ID NO: 356) corresponding to amino acids 809-843 of C03950_{—}3_P31 (SEQ. ID NO:228), wherein first amino acid sequence, second amino acid sequence and third amino acid sequence are contiguous and in a sequential order.

In some embodiments, this invention provides an isolated polypeptide comprising a head of C03950_{—}3_P31 (SEQ. ID NO:228), comprising a polypeptide being at least about 70%, optionally at least about 80%, preferably at least about 85%, more preferably at least about 90% and most preferably at least about 95% homologous to the sequence MAAARDPPEVSLREATQRKLRRFSELRGKLVARGEFWDIVAITAADEKQELAYNQQLSEKLK RICELPLGVQYHVFVDPAGAKIGNGGSTLCALQCLEKLYGDKWNSFTILLIHS (SEQ. ID NO: 379) of C03950_{—}3_P31 (SEQ. ID NO:228).

In some embodiments, this invention provides an isolated polypeptide comprising an edge portion of C03950_{—}3_P31 (SEQ. ID NO:228), comprising an amino acid sequence being at least about 70%, optionally at least about 80%, preferably at least about 85%, more preferably at least about 90% and most preferably at least about 95% homologous to the sequence AKSRPSHYPVSSVYTETLICKKNEDRFGMWIEYLRR (SEQ. ID NO: 356) of C03950_{—}3_P31 (SEQ. ID NO:228).

In some embodiments, the isolated chimeric proteins or polypeptides of the invention comprise an amino acid sequence corresponding to or homologous to C03950_{—}3_P33 (SEQ. ID NO:229).

In some embodiments, such isolated chimeric proteins or polypeptides comprise a first amino acid sequence being at least about 90% homologous to amino acids 1-691 of TNI3K_HUMAN (SEQ. ID NO: 396), which also corresponds to amino acids 1-691 of C03950_{—}3_P33 (SEQ. ID NO:229), and a second amino acid sequence being at least about 70%, optionally at least about 80%, preferably at least about 85%, more preferably at least about 90% and most preferably at least about 95% homologous to a polypeptide having the sequence RYFFPK (SEQ. ID NO: 364) corresponding to amino acids 692-697 of C03950_{—}3_P33 (SEQ. ID NO:229), wherein said first amino acid sequence and second amino acid sequence are contiguous and in a sequential order.

In some embodiments, this invention provides an isolated polypeptide comprising an edge portion of C03950_{—}3_P33 (SEQ. ID NO:229), comprising an amino acid sequence being at least about 70%, optionally at least about 80%, preferably at least about 85%, more preferably at least about 90% and most preferably at least about 95% homologous to the sequence RYFFPK (SEQ. ID NO: 364) of C03950_{—}3_P33 (SEQ. ID NO:229).

In some embodiments, such isolated chimeric proteins or polypeptides comprise a first amino acid sequence being at least about 90% homologous to amino acids 18-367 of Q6MZS9_HUMAN (SEQ. ID NO:211), which also corresponds to amino acids 1-350 of C03950_{—}3_P33 (SEQ. ID NO:229), a bridging amino acid I corresponding to amino acid 351 of C03950_{—}3_P33 (SEQ. ID NO:229), a second amino acid sequence being at least about 90% homologous to amino acids 369-484 of Q6MZS9_HUMAN (SEQ. ID NO:211), which also corresponds to amino acids 352-467 of C03950_{—}3_P33 (SEQ. ID NO:229), a bridging amino acid N corresponding to amino acid 468 of C03950_{—}3_P33 (SEQ. ID NO:229), and a third amino acid sequence being at least about 90% homologous to amino acids 486-714 of Q6MZS9_HUMAN (SEQ. ID NO:211), which also corresponds to amino acids 469-697 of C03950_{—}3_P33 (SEQ. ID NO:229), wherein said, first amino acid sequence, bridging amino acid, second amino acid sequence, bridging amino acid and third amino acid sequence are contiguous and in a sequential order.

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSETILLIHS (SEQ. ID NO: 379) corresponding to amino acids 1-114 of C03950_{—}3_P33 (SEQ. ID NO:229), a second amino acid sequence being at least about 90% homologous to amino acids 14-590 of NP_{—}057062 (SEQ. ID NO:210), which also corresponds to amino acids 115-691 of C03950_{—}3_P33 (SEQ. ID NO:229), a third amino acid sequence being at least about 70%, optionally at least about 80%, preferably at least about 85%, more preferably at least about 90% and most preferably at least about 95% homologous to a polypeptide having the sequence RYFFPK (SEQ. ID NO: 364) corresponding to amino acids 692-697 of C03950_{—}3_P33 (SEQ. ID NO:229), wherein said first amino acid sequence, second amino acid sequence and third amino acid sequence are contiguous and in a sequential order.

In some embodiments, this invention provides an isolated polypeptide comprising a head of C03950_{—}3_P33 (SEQ. ID NO:229), comprising a polypeptide being at least about 70%, optionally at least about 80%, preferably at least about 85%, more preferably at least about 90% and most preferably at least about 95% homologous to the sequence MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHS (SEQ. ID NO: 379) of C03950_{—}3_P33 (SEQ. ID NO:229).

In some embodiments, this invention provides an isolated polypeptide comprising an edge portion of C03950_{—}3_P33 (SEQ. ID NO:229), comprising an amino acid sequence being at least about 70%, optionally at least about 80%, preferably at least about 85%, more preferably at least about 90% and most preferably at least about 95% homologous to the sequence RYFFPK (SEQ. ID NO: 364) of C03950_{—}3_P33 (SEQ. ID NO:229).

In some embodiments, the isolated chimeric proteins or polypeptides of the invention comprise an amino acid sequence corresponding to or homologous to C03950_{—}3_P35 (SEQ. ID NO:230).

In some embodiments, such isolated chimeric proteins or polypeptides comprise a first amino acid sequence being at least about 90% homologous to amino acids 1-657 of TNI3K_HUMAN (SEQ. ID NO: 396), which also corresponds to amino acids 1-657 of C03950_{—}3_P35 (SEQ. ID NO:230), and a second amino acid sequence being at least about 70%, optionally at least about 80%, preferably at least about 85%, more preferably at least about 90% and most preferably at least about 95% homologous to a polypeptide having the sequence YGSFVLIYPWTFRRNYSCNTSEGFPLDEPSPFEI (SEQ. ID NO: 372) corresponding to amino acids 658-691 of C03950_{—}3_P35 (SEQ. ID NO:230), wherein said first amino acid sequence and second amino acid sequence are contiguous and in a sequential order.

In some embodiments, this invention provides an isolated polypeptide comprising an edge portion of C03950_{—}3_P35 (SEQ. ID NO:230), comprising an amino acid sequence being at least about 70%, optionally at least about 80%, preferably at least about 85%, more preferably at least about 90% and most preferably at least about 95% homologous to the sequence YGSFVLIYPWTFRRNYSCNTSEGFPLDEPSPFEI (SEQ. ID NO: 372) of C03950_{—}3_P35 (SEQ. ID NO:230).

In some embodiments, such isolated chimeric proteins or polypeptides comprise a first amino acid sequence being at least about 90% homologous to amino acids 18-367 of Q6MZS9_HUMAN (SEQ. ID NO:211), which also corresponds to amino acids 1-350 of C03950_{—}3_P35 (SEQ. ID NO:230), a bridging amino acid I corresponding to amino acid 351 of C03950_{—}3_P35 (SEQ. ID NO:230), a second amino acid sequence being at least about 90% homologous to amino acids 369-484 of Q6MZS9_HUMAN (SEQ. ID NO:211), which also corresponds to amino acids 352-467 of C03950_{—}3_P35 (SEQ. ID NO:230), a bridging amino acid N corresponding to amino acid 468 of C03950_{—}3_P35 (SEQ. ID NO:230), a third amino acid sequence being at least about 90% homologous to amino acids 486-674 of Q6MZS9_HUMAN (SEQ. ID NO:211), which also corresponds to amino acids 469-657 of C03950_{—}3_P35 (SEQ. ID NO:230), and a fourth amino acid sequence being at least about 70%, optionally at least about 80%, preferably at least about 85%, more preferably at least about 90% and most preferably at least about 95% homologous to a polypeptide having the sequence YGSFVLIYPWTFRRNYSCNTSEGFPLDEPSPFEI (SEQ. ID NO: 372) corresponding to amino acids 658-691 of C03950_{—}3_P35 (SEQ. ID NO:230), wherein said , first amino acid sequence, bridging amino acid, second amino acid sequence, bridging amino acid, third amino acid sequence and fourth amino acid sequence are contiguous and in a sequential order.

In some embodiments, this invention provides an isolated polypeptide comprising an edge portion of C03950_{—}3_P35 (SEQ. ID NO:230), comprising an amino acid sequence being at least about 70%, optionally at least about 80%, preferably at least about 85%, more preferably at least about 90% and most preferably at least about 95% homologous to the sequence YGSFVLIYPWTFRRNYSCNTSEGFPLDEPSPFEI (SEQ. ID NO: 372) of C03950_{—}3_P35 (SEQ. ID NO:230).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence—MAAARDPPEVSLREATQRKLRRFSELRGICLVARGEFWDIVAITAADEKQELAYNQQLSEICLK RICELPLGVQYHVFVDPAGAKIGNGGSTLCALQCLEKLYGDKWNSFTILLIHS (SEQ. ID NO: 379) corresponding to amino acids 1-114 of C03950_{—}3_P35 (SEQ. ID NO:230), a second amino acid sequence being at least about 90% homologous to amino acids 14-556 of NP_{—}057062 (SEQ. ID NO:210), which also corresponds to amino acids 115-657 of C03950_{—}3_P35 (SEQ. ID NO:230), a third amino acid sequence being at least about 70%, optionally at least about 80%, preferably at least about 85%, more preferably at least about 90% and most preferably at least about 95% homologous to a polypeptide having the sequence YGSFVLIYPWTFRRNYSCNTSEGFPLDEPSPFEI (SEQ. ID NO: 372) corresponding to amino acids 658-691 of C03950_{—}3_P35 (SEQ. ID NO:230), wherein said amino acid sequence, second amino acid sequence and third amino acid sequence are contiguous and in a sequential order.

In some embodiments, this invention provides an isolated polypeptide comprising a head of C03950_{—}3_P35 (SEQ. ID NO:230), comprising a polypeptide being at least about 70%, optionally at least about 80%, preferably at least about 85%, more preferably at least about 90% and most preferably at least about 95% homologous to the sequence—MAAARDPPEVSLREATQRKLRRFSELRGICLVARGEFWDIVAITAADEKQELAYNQQLSEICLK RKELPLGVQYHVFVDPAGAKIGNGGSTLCALQCLEKLYGDKWNSFTILLIHS (SEQ. ID NO: 379) of C03950_{—}3_P35 (SEQ. ID NO:230).

According to yet a further embodiment, the present invention now discloses a novel cluster designated herein R15601, comprising novel amino acid and nucleic acid sequences that are variants of the known protein cardiomyopathy associated 4 (SEQ. ID NO:267).

The novel polynucleotides and polypeptides described by the present invention are useful as diagnostic markers, preferably as serum markers.

Surprisingly, the present invention now shows that the R15601 variants are expressed specifically in heart tissues, and thus can indicate the onset, severity or prognosis of cardiovascular disease in a subject, and can be used for the selection of treatment, treatment monitoring, diagnosis or prognosis assessment of any cardiovascular disease, including, inter alia, myocardial infarct, acute coronary syndrome, coronary artery disease, angina pectoris (stable and unstable), cardiomyopathy, myocarditis, congestive heart failure or any type of heart failure, reinfarction, assessment of thrombolytic therapy, assessment of myocardial infarct size, differential diagnosis between heart-related versus lung-related conditions (such as pulmonary embolism), the differential diagnosis of Dyspnea, cardiac valves related conditions, vascular disease, or any combination thereof, as is described in a greater detail below.

In some embodiments, the isolated chimeric proteins or polypeptides of the invention comprise an amino acid sequence corresponding to or homologous to R15601_P2 (SEQ. ID NO:268).

In some embodiments, such isolated chimeric proteins or polypeptides comprise a first amino acid sequence being at least about 70%, optionally at least about 80%, preferably at least about 85%, more preferably at least about 90% and most preferably at least about 95%, homologous to a polypeptide having the sequence MPRKDRNSSRAESAQCQVLSCVIEIGILLMAREIAVVVLPLSQ (SEQ. ID NO: 388) corresponding to amino acids 1-42 of R15601_P2 (SEQ. ID NO:268), and a second amino acid sequence being at least about 90% homologous to amino acids 57-931 of NP_{—}775259 (SEQ. ID NO: 397), which also corresponds to amino acids 43-917 of R15601_P2 (SEQ ID NO:268), wherein said first amino acid sequence and second amino acid sequence are contiguous and in a sequential order.

In some embodiments, this invention provides an isolated polypeptide comprising a head of R15601_P2 (SEQ. ID NO:268), comprising a polypeptide being at least about 70%, optionally at least about 80%, preferably at least about 85%, more preferably at least about 90% and most preferably at least about 95% homologous to the sequence MPRKDRNSSRAESAQCQVLSCVIHGILLMAREIAVVVLPLSQ (SEQ. ID NO: 388) of R15601_P2 (SEQ. ID NO:268).

In some embodiments, the isolated chimeric proteins or polypeptides of the invention comprise an amino acid sequence corresponding to or homologous to R15601_P3 (SEQ. ID NO:269).

In some embodiments, such isolated chimeric proteins or polypeptides comprise a first amino acid sequence being at least about 90% homologous to amino acids 1-565 of NP_{—}775259 (SEQ. ID NO: 397), which also corresponds to amino acids 1-565 of R15601_P3 (SEQ. ID NO:269), and a second amino acid sequence being at least about 70%, optionally at least about 80%, preferably at least about 85%, more preferably at least about 90% and most preferably at least about 95% homologous to a polypeptide having the sequence VGESGPTTNLRKGLLGPDPQGMDPSLPPGSTPYPONMIGYFPLSGKIFT (SEQ. ID NO: 389) corresponding to amino acids 566-615 of R15601 P3 (SEQ. ID NO:269), wherein said first amino acid sequence and second amino acid sequence are contiguous and in a sequential order.

In some embodiments, this invention provides an isolated polypeptide comprising an edge portion of R15601_P3 (SEQ. ID NO:269), comprising an amino acid sequence being at least about 70%, optionally at least about 80%, preferably at least about 85%, more preferably at least about 90% and most preferably at least about 95% homologous to the sequence VGESGPTTNLRKGLLGPDPQGMDPSLPPGSTPYPCINMIGYFPLSGPHFT (SEQ. ID NO: 389) of R15601_P3 (SEQ. ID NO:269).

According to an additional embodiment, the present invention now discloses a novel cluster designated herein T11811, comprising novel amino acid and nucleic acid sequences that are variants of the known protein Myosin regulatory light chain 2, atrial isoform (SwissProt accession identifier MLRA_HUMAN (SEQ. ID NO: 398).

The novel polynucleotides and polypeptides described by the present invention are useful as diagnostic markers, preferably as serum markers.

Surprisingly, the present invention now shows that the T11811 variants are expressed specifically in heart tissue, and thus can indicate the onset, severity or prognosis of cardiovascular disease in a subject, and can be used for the selection of treatment, treatment monitoring, diagnosis or prognosis assessment of any cardiovascular disease, including, inter alia, myocardial infarct, acute coronary syndrome, coronary artery disease, angina pectoris (stable and unstable), cardiomyopathy, myocarditis, congestive heart failure or any type of heart failure, reinfarction, assessment of thrombolytic therapy, assessment of myocardial infarct size, differential diagnosis between heart-related versus lung-related conditions (such as pulmonary embolism), the differential diagnosis of Dyspnea, cardiac valves related conditions, vascular disease, or any combination thereof, as is described in a greater detail below.

In some embodiments, the isolated chimeric proteins or polypeptides of the invention comprise an amino acid sequence corresponding to or homologous to T11811_P2 (SEQ. ID NO:303).

In some embodiments, such isolated chimeric proteins or polypeptides comprise a first amino acid sequence being at least about 90% homologous to amino acids 1-142 of MLRA_HUMAN (SEQ. ID NO: 398), which also corresponds to amino acids 1-142 of T11811_P2 (SEQ. ID NO:303), a second amino acid sequence being at least about 70%, optionally at least about 80%, preferably at least about 85%, more preferably at least about 90% and most preferably at least about 95% homologous to a polypeptide having the sequence VRLPSPFNTHPQHLLWAFTHDPEPSTSEAVAGR (SEQ. ID NO: 390) corresponding to amino acids 143-175 of T11811_P2 (SEQ. ID NO:303), and a third amino acid sequence being at least about 90% homologous to amino acids 143-175 of MIRA_HUMAN (SEQ. ID NO: 398), which also corresponds to amino acids 176-208 of T11811_P2 (SEQ. ID NO:303), wherein said first amino acid sequence, second amino acid sequence and third amino acid sequence are contiguous and in a sequential order.

In some embodiments, this invention provides an isolated polypeptide comprising an edge portion of T11811_P2 (SEQ. ID NO:303), comprising an amino acid sequence being at least about 70%, optionally at least about 80%, preferably at least about 85%, more preferably at least about 90% and most preferably at least about 95% homologous to the sequence VRLPSPFNTHPQHLLWAFTHDPEPSTSEAVAGR (SEQ. ID NO: 390) of T11811_P2 (SEQ. ID NO:303).

In some embodiments, the isolated chimeric proteins or polypeptides of the invention comprise an amino acid sequence corresponding to or homologous to T11811_P4 (SEQ. ID NO:304).

In some embodiments, such isolated chimeric proteins or polypeptides comprise a first amino acid sequence being at least about 90% homologous to amino acids 1-125 of MLRA_HUMAN (SEQ. ID NO: 398), which also corresponds to amino acids 1-125 of T11811_P4 (SEQ. ID NO:304), and a second amino acid sequence being at least about 70%, optionally at least about 80%, preferably at least about 85%, more preferably at least about 90% and most preferably at least about 95% homologous to a polypeptide having the sequence DQPFPAPWEPPYPPSLCSHSPAVSCSDPPHPPGSSSFS (SEQ. ID NO: 391) corresponding to amino acids 126-163 of T11811_P4 (SEQ. II) NO:304), wherein said first amino acid sequence and second amino acid sequence are contiguous and in a sequential order.

In some embodiments, this invention provides an isolated polypeptide comprising an edge portion of T11811_P4 (SEQ. ID NO:304), comprising an amino acid sequence being at least about 70%, optionally at least about 80%, preferably at least about 85%, _{more pre}fₑrably at least about 90% and most preferably at least about 95% homologous to the sequence DQPFPAPWEPPYPPSLCSHSPAVSCSDPPHPPGSSSFS (SEQ. ID NO: 391) of T11811_P4 (SEQ. ID NO:304).

In some embodiments, the isolated chimeric proteins or polypeptides of the invention comprise an amino acid sequence corresponding to or homologous to T11811_P7 (SEQ. ID NO:305).

In some embodiments, such isolated chimeric proteins or polypeptides comprise a first amino acid sequence being at least about 90% homologous to amino acids 1-39 of MLRA_HUMAN (SEQ. ID NO: 398), which also corresponds to amino acids 1-39 of T11811_P7 (SEQ. ID NO:305), a second amino acid sequence being at least about 70%, oₚtionally at least about 80%, preferably at least about 85%, more preferably at least about 90% and most preferably at least about 95% homologous to a polypeptide having the sequence VSPPPPTFPRAGGCSHLKAPIPQ (SEQ. ID NO:

392) corresponding to amino acids 40-62 of T11811_P7 (SEQ. ID NO:305), and a third amino acid sequence being at least about 90% homologous to amino acids 40-175 of MLRA_HUMAN (SEQ. ID NO: 398), which also corresponds to amino acids 63-198 of T11811_P7 (SEQ. ID NO:305), wherein said first amino acid sequence, second amino acid sequence and third amino acid sequence are contiguous and in a sequential order.

In some embodiments, this invention provides an isolated polypeptide comprising an edge portion of T11811_P7 (SEQ. ID NO:305), comprising an amino acid sequence being at least about 70%, optionally at least about 80%, preferably at least about 85%, more preferably at least about 90% and most preferably at least about 95% homologous to the sequence VSPPPPTFPRAGGCSHLKAPIPQ (SEQ. ID NO: 392) of T11811_P7 (SEQ. ID NO:305).

In some embodiments, the isolated chimeric proteins or polypeptides of the invention comprise an amino acid sequence corresponding to or homologous to T11811_P8 (SEQ. ID NO:306).

In some embodiments, such isolated chimeric proteins or polypeptides comprise a first amino acid sequence being at least about 90% homologous to amino acids 1-126 of MLRA_HUMAN (SEQ. ID NO: 398), which also corresponds to amino acids 1-126 of T11811_P8 (SEQ. ID NO:306), and a second amino acid sequence being at least about 70%, optionally at least about 80%, preferably at least about 85%, more preferably at least about 90% and most preferably at least about 95% homologous to a polypeptide having the sequence WSRCSP (SEQ. ID NO: 393) corresponding to amino acids 127-132 of T11811_P8 (SEQ. ID NO:306), wherein said first amino acid sequence and second amino acid sequence are contiguous and in a sequential order.

In some embodiments, this invention provides an isolated polypeptide comprising an edge portion of T11811_P3 (SEQ. ID NO:306), comprising an amino acid sequence being at least about 70%, optionally at least about 80%, preferably at least about 85%, more preferably at least about 90% and most preferably at least about 95% homologous to the sequence WSRCSP (SEQ. ID NO: 393) of T11811_P8 (SEQ. ID NO:306).

According to certain embodiments, the polypeptides of this invention comprise variants of known proteins, and in other embodiments the polypeptides of this invention comprise splice variants of native proteins expressed in a given subject. In some embodiments, the polypeptides may be obtained through known protein evolution techniques available in the art. In other embodiments, the polypeptides of this invention may be obtained via rational design, based on a particular native polypeptide sequence.

According to another aspect the present invention provides antibodies or antibody fragments specifically interacting with or recognizing a polypeptide of this invention.

According to certain embodiments, the antibody recognizes one or more epitopes (antigen determinants) contained within the polypeptides of this invention, wherein such that binding of the antibody to an epitope distinguish between the splice variants of the present invention and a known polypeptide or protein. Reference to the antibody property of “specific interaction” or “recognition” is to be understood as including covalent and non-covalent associations with a variance of affinity over several orders of magnitude. These terms are to be understood as relative with respect to an index molecule, for which the antibody is thought to have little to no specific interaction or recognition. In one embodiment, the antibodies specifically interact or recognize a particular antigen determinant.

In certain embodiments, the antibodies or antibody fragments of this invention recognize or interact with a polypeptide or protein of the invention, while not substantially recognize or interact with other molecules, even when present in the same sample, for example a biological sample. According to one embodiment, the antibodies of this invention have a specificity such that the specific interaction with or binding to the antigen is at least about 2, or in another embodiment, at least about 5, or in still further embodiment, at least about 10-fold greater than interaction or binding observed under the same reaction conditions with a molecule that does not include the antigenic determinant.

According to certain embodiments, the antibodies are useful in detecting qualitative and/or quantitative changes in the expression of the polypeptides or polynucleotides of this invention. In some embodiments, changes in expression are associated with a particular disease or disorder, such that detection of the changes comprises a diagnostic method of the present invention.

According to other embodiments, the present invention provides an antibody capable of specifically binding to at least one epitope of a polypeptide comprising an amino acid sequence as set forth in any one of SEQ. ID NOs: 57-59, 63, 76, 77, 134-143, 212-230, 268-269, 303-308, 326-394.

According to additional aspect the present invention provides a diagnostic kit for detecting a disease, comprising markers and reagents for detecting qualitative and/or quantitative changes in the expression of a polypeptide or a polynucleotide of this invention.

According to on embodiment, the kit comprises markers and reagents for detecting the changes by employing a NAT-based technology. In one embodiment, the NAT-based assay is selected from the group consisting of a PCR, Real-Time PCR, LCR, Self-Sustained Synthetic Reaction, Q-Beta Replicase, Cycling Probe Reaction, Branched DNA, RFLP analysis, DGGE/TGGE, Single-Strand Conformation Polymorphism, Dideoxy Fingerprinting, Microarrays, Fluorescence In Situ Hybridization or Comparative Genomic Hybridization.

According to certain currently preferred embodiments, the kit comprises at least one nucleotide probe or primer. In one embodiment, the kit comprises at least one primer pair capable of selectively hybridizing to a nucleic acid sequence according to the teaching of the present invention. In another embodiment, the kit comprises at least one oligonucleotide capable of selectively hybridizing to a nucleic acid sequence according to the teaching of the present invention.

According to other currently preferred embodiments, the kit comprises an antibody capable of recognizing or interacting with a polypeptide or protein of the present invention. According to certain embodiments, the kit further comprises at least one reagent for performing an ELISA, an RIA, a slot blot, an immunohistochemical assay, FACS, in-vivo imaging, a radio-imaging assay, or a Western blot.

The present invention further provides diagnostic methods for screening for a disease, disorder or conditions, comprising the detection of a polypeptide or polynucleotide of this invention, whereby expression, or relative changes in expression of the polypeptide or polynucleotide herald the onset, severity, or prognosis of an individual with regard to a particular disease, disorder or condition. The detection may comprise detection of the expression of a specific splice variant, or other polypeptide or polynucleotide of this invention, via any means known in the art, and as described herein.

As used herein, the term “screening for a disease” encompasses diagnosing the presence of a disease, its prognosis and/or severity, as well as selecting a treatment and monitoring the treatment of the disease. According to certain currently preferred embodiments, the disease is a marker-detectable disease, wherein the marker is a polynucleotide, polypeptide or protein according to the present invention.

Thus, according to certain aspects, the present invention provides methods for screening for a marker detectable disease, comprising detecting in a subject or in a sample obtained from the subject at least one transcript and/or protein or polypeptide being a member of a cluster selected from the group consisting of cluster N43992, cluster D12115, cluster C03950, cluster R15601, cluster T11811, or any combination thereof. According to certain currently preferred embodiments, the method comprises detecting the expression of a splice variant transcript or a product thereof.

According to one aspect, the present invention provide a method for screening for a marker detectable disease in a subject, comprising (a) obtaining a sample from the subject and (b) detecting in the sample at least one polynucleotide and/or polypeptide being a member of a cluster selected from the group consisting of cluster C03950, cluster R15601, cluster T11811, or any combination thereof. According to one embodiment, the presence of the polynucleotide or polypeptide in the sample is indicative of the presence of the disease and/or its severity and/or its progress. According to another embodiment, a change in the level of the polynucleotide or polypeptide in the sample compared to its level in a sample obtained from a healthy subject is indicative of the presence of the disease and/or its severity. According to another embodiment, a change in the level of the polynucleotide or polypeptide in the sample compared to its level in a sample previously obtained from said subject is indicative of the presence of the disease, its severity and/or the progress of the disease.

According to one embodiment, the present invention provides a method for screening for a cardiovascular disease in a subject, comprising (a) obtaining a sample from the subject and (b) detecting in the sample at least one polypeptide being a member of a cluster selected from the group consisting of cluster C03950, cluster R15601, cluster T11811, or any combination thereof. According to one embodiment, the presence of the polypeptide in the sample is indicative of the presence of the disease and/or its severity and/or its progress. According to another embodiment, a change in the level of the polypeptide in the sample compared to its level in a sample obtained from a healthy subject is indicative of the presence of the disease and/or its severity. According to another embodiment, a change in the level of the polypeptide in the sample compared to its level in a sample previously obtained from said subject is indicative of the presence of the disease, its severity and/or the progress of the disease. According to currently preferred embodiments, the sample is a serum sample.

According to other embodiments, the cardiovascular disease include inter alia, myocardial infarct, acute coronary syndrome, coronary artery disease, angina pectoris (stable and unstable), cardiomyopathy, myocarditis, congestive heart failure or any type of heart failure and reinfarction. According to other embodiments, the method is useful for the assessment of thrombolytic therapy, assessment of myocardial infarct size, differential diagnosis between heart-related versus lung-related conditions (such as pulmonary embolism), the differential diagnosis of Dyspnea, cardiac valves related conditions, vascular disease, or any combination thereof. In further embodiments, the polypeptides of any one of the clusters C03950, R15601, T11811, or a combination thereof, are useful in the diagnosis, treatment or assessment of the prognosis of a subject with congestive heart failure (CHF). According to still other embodiments, they are useful in the diagnosis, treatment or assessment of the prognosis of a subject with sudden cardiac death, from arrhythmia or any other heart related reason; rejection of a transplanted heart; conditions that lead to heart failure including but not limited to myocardial infarction, angina, arrhythmias, valvular diseases, atrial and/or ventricular septal defects; conditions that cause atrial and or ventricular wall volume overload, including but not limited to systemic arterial hypertension, pulmonary hypertension and pulmonary embolism; conditions which have similar clinical symptoms as heart failure and as states that cause atrial and or ventricular pressure-overload, where the differential diagnosis between these conditions to the latter is of clinical importance including but not limited to breathing difficulty and/or hypoxia due to pulmonary disease, anemia or anxiety.

Each polypeptide of the C03950 variants, R15601 variants, and/or T11811 variants described herein as a marker for cardiovascular conditions, can be used alone or in combination with one or more other variant markers described herein, and/or in combination with known markers for cardiovascular conditions, including but not limited to Heart-type fatty acid binding protein (H-FABP), Angiotensin, C-reactive protein (CRP), myeloperoxidase (MPO), and/or in combination with the known protein(s) for the variant marker as described herein.

The present invention further discloses that surprisingly, detecting in a subject at least one polypeptide or polynucleotide of cluster N43992, including the known N43992 polypeptide and/or polynucleotide sequences as well as variants thereof as disclosed in the present invention, are indicative of cancer, particularly lung cancer. Detecting the presence of the polynucleotide or polypeptide in the subject or detecting a relative change in their expression and/or level compared to a healthy subject or compared to their expression and/or level in said subject at an earlier stage is indicative of the presence, onset, severity or prognosis, and/or staging, and/or progression, of lung cancer in said subject. These polynucleotides and polypeptides of cluster N43992 are also useful for treatment selection and treatment monitoring of lung cancer, which may be an invasive lung cancer and/or metastatic lung cancer.

Thus, according to another aspect, the present invention provides a method for screening for a cancer in a subject, comprising detecting in the subject at least one polynucleotide and/or polypeptide being a member of cluster N43992.

According to one embodiment, the polypeptide or polynucleotide is at least 85% homologous to the wild type protein DLL3 or a polynucleotide encoding same, respectively, or a fragment thereof. As the wild type DLL3 protein is a type I membrane protein it is used as a diagnostic marker preferably with in vivo imaging technologies, including but not limited to magnetic resonance imaging, computed tomography scanning, PET, SPECT and the like. Optionally, according to the present invention, the wild type DLL3 protein diagnostic marker is used as IHC marker.

According to another embodiment, the polypeptide or polynucleotide is at least 85% homologous to a secreted splice variant of protein DLL3 or a polynucleotide encoding same, respectively, or a fragment thereof. According to this embodiment, the method for screening for a cancer is performed in vitro with a sample obtained from the subject.

According to another aspect, the present invention provides a method for screening for cancer in a subject, comprising detecting in the subject a polypeptide comprising an amino acid sequence at least 85% homologous to the amino acid sequence set forth in any one of SEQ. ID NOs: 54-59, 63, 76 and 77. According to one embodiment, the method comprises detecting a polypeptide comprising an amino acid sequence as set forth in any one of SEQ. ID NOs: 54-59, 63, 76 and 77.

According to yet another aspect, the present invention provides a method for screening for cancer in a subject, comprising detecting in the subject a polynucleotide comprising a nucleic acid sequence at least 85% homologous to the nucleic acid sequence set forth in any one of SEQ. ID NOs: 37-39, 40-53, 74-75. According to one embodiment, the method comprises detecting a polynucleotide comprising a nucleic acid sequence as set forth in any one of SEQ. ID NOs: 37-39, 40-53, 74-75.

According to one embodiment, the cancer is a lung cancer, wherein the lung cancer can be invasive or metastatic.

In other aspects, the present invention discloses that detection of polypeptides or polynucleotides of cluster D12115 variants, or relative changes in expression and/or level of these variants and their products is indicative of the presence, onset, severity or prognosis, and/or staging, and/or progression of cancer, including but not limited to ovarian cancer, lung cancer or breast cancer, in a subject. In some embodiments, the polypeptides, polynucleotides and/or methods of this invention may be useful in the treatment selection and monitoring, diagnosis or prognosis assessment of cancer, including but not limited to ovarian cancer, lung cancer or breast cancer, or ovarian, breast or lung cancer invasion and metastasis.

With regard to lung cancer, the disease is selected from the group consisting of invasive or metastatic lung cancer; squamous cell lung carcinoma, lung adenocarcinoma, carcinoid, small cell lung cancer or non-small cell lung cancer; detection of overexpression in lung metastasis (vs. primary tumor); detection of overexpression in lung cancer, for example non small cell lung cancer, for example adenocarcinoma, squamous cell cancer or carcinoid, or large cell carcinoma; identification of a metastasis of unknown origin which originated from a primary lung cancer; assessment of a malignant tissue residing in the lung that is from a non-lung origin, including but not limited to: osteogenic and soft tissue sarcomas; colorectal, uterine, cervix and corpus tumors; head and neck, breast, testis and salivary gland cancers; melanoma; and bladder and kidney tumors; distinguishing between different types of lung cancer, therefore potentially affecting treatment choice (e.g. small cell vs. non small cell tumors); analysis of unexplained dyspnea and/or chronic cough and/or hemoptysis; differential diagnosis of the origin of a pleural effusion; diagnosis of conditions which have similar symptoms, signs and complications as lung cancer and where the differential diagnosis between them and lung cancer is of clinical importance including but not limited to: non-malignant causes of lung symptoms and signs, including but not limited to: lung lesions and infiltrates, wheeze, stridor, tracheal obstruction, esophageal compression, dysphagia, recurrent laryngeal nerve paralysis, hoarseness, phrenic nerve paralysis with elevation of the hemidiaphragm and Horner syndrome; or detecting a cause of any condition suggestive of a malignant tumor including but not limited to anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.

The polypeptides and/or polynucleotides of cluster D12115 and/or N43992 used as markers for lung cancer can be used alone or in combination with one or more alternative polynucleotides or polypeptides described herein, and/or in combination with known markers for lung cancer, including but not limited to CEA, CA15-3, Beta-2-microglobulin, CA19-9, TPA, and/or in combination with the known protein(s) for the variant marker as described herein.

With regard to ovarian cancer, the polypeptides and/or polynucleotide of cluster D12115 of the present invention can be used in the diagnosis, treatment or prognostic assessment of invasive or metastatic ovarian cancer; correlating stage and malignant potential; identification of a metastasis of unknown origin which originated from a primary ovarian cancer; differential diagnosis between benign and malignant ovarian cysts; diagnosing a cause of infertility, for example differential diagnosis of various causes thereof; detecting of one or more non-ovarian cancer conditions that may elevate serum levels of ovary related markers, including but not limited to: cancers of the endometrium, cervix, fallopian tubes, pancreas, breast, lung and colon; nonmalignant conditions such as pregnancy, endometriosis, pelvic inflammatory disease and uterine fibroids; diagnosing conditions which have similar symptoms, signs and complications as ovarian cancer and where the differential diagnosis between them and ovarian cancer is of clinical importance including but not limited to: non-malignant causes of pelvic mass, including, but not limited to: benign (functional) ovarian cyst, uterine fibroids, endometriosis, benign ovarian neoplasms and inflammatory bowel lesions; determining a cause of any condition suggestive of a malignant tumor including but not limited to anorexia, cachexia, weight loss, fever, hypercalcemia, skeletal or abdominal pain, paraneoplastic syndrome, or ascites.

The polypeptides and/or polynucleotides of cluster D12115 used in the diagnosis, treatment or prognostic assessment of ovarian cancer can be used alone or in combination with one or more polypeptides and/or polynucleotides of this invention, and/or in combination with known markers for ovarian cancer, including but not limited to CEA, CA125 (Mucin 16), CA72-4TAG, CA-50, CA 54-61, CA-195 and CA 19-9 in combination with CA-125, and/or in combination with the known protein(s) associated with the indicated polypeptide or polynucleotide, as described herein.

With regard to breast cancer, the polypeptides and/or polynucleotides of cluster D12115 are useful in determining a probable outcome in breast cancer; identification of a metastasis of unknown origin which originated from a primary breast cancer tumor; assessing lymphadenopathy, and in particular axillary lymphadenopathy; distinguishing between different types of breast cancer, therefore potentially affect treatment choice (e.g. as HER-2); differentially diagnosing between a benign and malignant breast mass; as a tool in the assessment of conditions affecting breast skin (e.g. Paget's disease) and their differentiation from breast cancer; differential diagnosis of breast pain or discomfort resulting from either breast cancer or other possible conditions (e.g. mastitis, Mondors syndrome); non-breast cancer conditions which have similar symptoms, signs and complications as breast cancer and where the differential diagnosis between them and breast cancer is of clinical importance including but not limited to: abnormal mammogram and/or nipple retraction and/or nipple discharge due to causes other than breast cancer, including but not limited to benign breast masses, melanoma, trauma and technical and/or anatomical variations; determining a cause of any condition suggestive of a malignant tumor including but not limited to anorexia, cachexia, weight loss, fever, hypercalcemia, paraneoplastic syndrome; or determining a cause of lymphadenopathy, weight loss and other signs and symptoms associated with breast cancer but originate from diseases different from breast cancer including but not limited to other malignancies, infections and autoimmune diseases.

Each variant marker of the present invention described herein as potential marker for breast cancer can be used alone or in combination with one or more other variant breast cancer described herein, and/or in combination with known markers for breast cancer, including but not limited to Calcitonin, CA15-3 (Mucin1), CA27-29, TPA, a combination of CA 15-3 and CEA, CA 27.29 (monoclonal antibody directed against MUC1), Estrogen 2 (beta), HER-2 (c-erbB2), and/or in combination with the known protein(s) for the variant marker as described herein.

According to certain embodiments, a combination of anyone of the polynucleotides or polypeptides markers of the present invention with another marker can be used for determining a ratio between a quantitative or semi-quantitative measurement of any marker described herein to any other marker described herein, and/or any other known marker, and/or any other marker. With regard to such a ratio between any marker described herein (or a combination thereof) and a known marker, the known marker preferably comprises the “known protein” as described in greater detail below with regard to each cluster or gene.

It is to be understood that any polynucleotide or polypeptide of this invention may be useful as a marker for a disease, disorder or condition, and such use is to be considered a part of this invention.

According to certain embodiments, detecting the expression of a polynucleotide or polypeptide according to the teaching of the present invention is performed by employing a NAT-based technology (optionally by employing at least one nucleotide probe or primer), or by employing an immunoassay (optionally by employing an antibody according to any of the embodiments described herein), respectively.

In some embodiments, this invention provides a method for screening for a disease in a subject, comprising detecting in the subject or in a sample obtained from said subject at least one polypeptide or polynucleotide selected from the group consisting of:
a. a polypeptide having an amino acid sequence as set forth in any one of SEQ. ID NOs: 54-59, 134-143, 212-230, 268-269, 303-308, or a homologue or a fragment thereof;
b. a polypeptide comprising a bridge, edge portion, tail, or head portion, wherein the polypeptide has an amino acid sequence as set forth in any one of SEQ. ID NOs: 326-394, or a homologue or a fragment thereof;
c. a polynucleotide having a nucleic acid sequence as set forth in any one of SEQ. ID NOs: 37-39, 74-75, 78-88, 156-170, 240-241, 276-281, or a homologue or a fragment thereof;
d. a polynucleotide comprising a node having a nucleic acid sequence as set forth in any one of SEQ. ID NOs: 40-53, 89-130, 171-208, 242-266, 282-301;
e. an oligonucleotide having a nucleic acid sequence as set forth in SEQ. ID NOs: 62, 64, 67, 68, 70, 71, 73, 146, 149, 152, 155, 233, 236, 239, 272, 275, 311, 314, 317, 320, 323-325.

According to one embodiment, detecting the presence of the polypeptide or polynucleotide is indicative of the presence of the disease and/or its severity and/or its progress. According to another embodiment, a change in the expression and/or the level of the polynucleotide or polypeptide compared to its expression and/or level in a healthy subject or a sample obtained therefrom is indicative of the presence of the disease and/or its severity and/or its progress. According to a further embodiment, a change in the expression and/or level of the polynucleotide or polypeptide compared to its level and/or expression in said subject or in a sample obtained therefrom at earlier stage is indicative of the progress of the disease. According to still further embodiment, detecting the presence and/or relative change in the expression and/or level of the polynucleotide or polypeptide is useful for selecting a treatment and/or monitoring a treatment of the disease.

According to one embodiment, detecting a polynucleotide of the invention comprises employing a primer pair, comprising a pair of isolated oligonucleotides capable of specifically hybridizing to at least a portion of a polynucleotide having a nucleic acid sequence as set forth in SEQ. ID NOs: 62, 67, 70, 73, 146, 149, 152, 155, 233, 236, 239, 272, 275, 311, 314, 317, 320, 323 or polynucleotides homologous thereto.

According to another embodiment, detecting a polynucleotide of the invention comprises employing a primer pair, comprising a pair of isolated oligonucleotides as set forth in SEQ. ID NOs:60-61, 65-66, 69, 72, 144-145, 147-148, 150-151, 153-154, 231-232, 234-235, 237-238, 270-271, 273-274, 309-310, 312-313, 315-316, 318-319, 321-322.

According to further embodiment, detecting a polypeptide of the invention comprises employing an antibody capable of specifically binding to at least one epitope of a polypeptide comprising an amino acid sequence as set forth in any one of SEQ. ID NOs: 57-59, 63, 76, 77, 134-143, 212-230, 268-269, 303-308, 326-394.

In some embodiments, a method of this invention may make use of a polynucleotide, polypeptide, vector, antibody, biomarker, or combination thereof, as described herein, including any embodiments thereof.

In some embodiments, the methods of this invention are conducted on a whole body. According to other embodiments, the methods of the present invention are conducted with a sample isolated from a subject having, predisposed to, or suspected of having the disease, disorder or condition. According to certain embodiments, the sample is a cell or tissue or a body fluid sample. In some embodiments, the methods are directed to the monitoring of disease progression and/or treatment efficacy and/or relapse of the indicated disease, disorder or condition.

In another embodiment, this invention provides methods for the selection of a particular therapy, or optimization of a given therapy for a disease, disorder or condition, the method comprising quantitatively and/or qualitatively determining or assessing expression of the polypeptides and/or polynucleotides, whereby differences in expression from an index sample, or a sample taken from a subject prior to the initiation of the therapy, or during the course of therapy, is indicative of the efficacy, or optimal activity of the therapy.

According to still further aspect, the present invention provides a method for detecting a splice variant nucleic acid sequence in a biological sample, comprising: hybridizing the isolated splice variant nucleic acid molecules or oligonucleotide fragments thereof of at least about 12 nucleotides to a nucleic acid material of the biological sample and detecting a hybridization complex; wherein the presence of the hybridization complex correlates with the presence of said splice variant nucleic acid sequence in the biological sample.

The nucleic acid sequences and/or amino acid sequences shown herein as embodiments of the present invention relate, in some embodiments, to their isolated form, as isolated polynucleotides (including for all transcripts), oligonucleotides (including for all segments, amplicons and primers), peptides (including for all tails, bridges, insertions or heads, optionally including other antibody epitopes as described herein) and/or polypeptides (including for all proteins). It should be noted that the terms “oligonucleotide” and “polynucleotide” and “nucleic acid molecule”, or “peptide” and “polypeptide” and “protein”, may optionally be used interchangeably.

All technical and scientific terms used herein should be understood to have the meaning commonly understood by a person skilled in the art to which this invention belongs, as well as any other specified description. The following references provide one of skill in the art with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows a schematic description of the cancer biomarker selection engine.

FIG. 2 shows a schematic illustration, depicting grouping of transcripts of a given cluster based on presence or absence of unique sequence regions.

FIG. 3 shows a schematic presentation of the oligonucleotide based microarray fabrication.

FIG. 4 shows a schematic summary of the oligonucleotide based microarray experimental flow.

FIG. 5 shows a schematic summary of quantitative real-time PCR analysis.

FIG. 6 shows a graph of cancer and cell-line vs. normal tissue expression for N43992.

FIGS. 7A-B is a histogram showing over expression of the Homo sapiens delta-like 3 (Drosophila) (DLL3) N43992 transcripts which are detectable by Taqman probes as depicted in sequence names N43992-T4 (SEQ. ID NO: 64) and N43992-T4II (SEQ. ID NO: 68) in normal and cancerous Lung tissues. FIG. 7A is a histogram showing the results using N43992-T4 (SEQ. ID NO: 64) probe. FIG. 7B is a histogram showing the results using N43992T4II (SEQ. ID NO: 68) probe.

FIG. 8A is a histogram showing over expression of the Homo sapiens delta-like 3 (Drosophila) (DLL3) N43992 transcripts which are detectable by amplicon as depicted in sequence name N43992_seg12WTF2R2 (SEQ. ID NO: 62) in normal and cancerous Lung tissues.

FIG. 8B is a histogram showing Expression of Homo sapiens delta-like 3 (Drosophila) (DLL3) N43992 transcripts which are detectable by Taqman probe as depicted in sequence names N43992T3 (SEQ. ID NO: 71) in normal and cancerous Lung tissues.

FIG. 9 is a histogram showing expression of Homo sapiens delta-like 3 (Drosophila) (DLL3) N43992 transcripts which are detectable by Taqman probe as depicted in sequence names N43992-T4 (SEQ. ID NO: 64) in different normal tissues.

FIG. 10A is a histogram showing over expression of the Homo sapiens delta-like 3 (Drosophila) (DLL3) N43992 transcripts which are detectable by amplicon as depicted in sequence name N43992_seg12WTF2R2 (SEQ. ID NO: 62) in different normal tissues.

FIG. 10B is a histogram showing expression of Homo sapiens delta-like 3 (Drosophila) (DLL3) N43992 transcripts which are detectable by Taqman probe as depicted in sequence names N43992T3 (SEQ. ID NO: 71) in different normal tissues.

FIG. 11 shows a graph of cancer and cell-line vs. normal tissue expression for D12115.

FIG. 12 is a histogram showing expression of Homo sapiens B-factor, properdin (BF) D12115 transcripts which are detectable by junction 0-2 and segment 6 in normal and cancerous ovary (FIG. 12A) and breast (FIG. 12B) tissues.

FIG. 13 is a histogram showing over expression of the Homo sapiens B-factor, properdin (BF) D12115 transcripts which are detectable by amplicon as depicted in sequence name D12115_seg4 (SEQ. ID NO: 146) in cancerous Breast samples relative to the normal samples.

FIG. 14 is a histogram showing expression of the Homo sapiens B-factor, properdin (BF) D12115 transcripts which are detectable by amplicon as depicted in sequence name D12115_seg4 (SEQ. ID NO: 146) in different normal samples.

FIG. 15 is a histogram showing over expression of the Homo sapiens B-factor, properdin (BF) D12115 transcripts which are detectable by amplicon as depicted in sequence name D12115_seg4 (SEQ. ID NO: 146) in cancerous ovarian samples relative to the normal samples.

FIG. 16 is a histogram showing over expression of the Homo sapiens B-factor, properdin (BF) D12115 transcripts which are detectable by amplicon as depicted in sequence name D12115_seg6 (SEQ. ID NO: 149) in cancerous Breast samples relative to the normal samples.

FIG. 17 is a histogram showing over expression of the Homo sapiens B-factor, properdin (BF) D12115 transcripts which are detectable by amplicon as depicted in sequence name D12115_seg6 (SEQ. ID NO: 149) in cancerous lung samples relative to the normal samples.

FIG. 18 is a histogram showing expression of the Homo sapiens B-factor, properdin (BF) D12115 transcripts which are detectable by amplicon as depicted in sequence name D12115_seg6 (SEQ. ID NO: 149) in different normal samples.

FIG. 19 is a histogram showing over expression of the Homo sapiens B-factor, properdin (BF) D12115 transcripts which are detectable by amplicon as depicted in sequence name D12115_seg6 (SEQ. ID NO: 149) in cancerous ovarian samples relative to the normal samples.

FIG. 20 is a histogram showing expression of the Homo sapiens B-factor, properdin (BF) D12115 transcripts which are detectable by amplicon as depicted in sequence name D12115 seg40WT (SEQ. ID NO: 152) in different normal samples.

FIG. 21 is a histogram showing expression of the Homo sapiens B-factor, properdin (BF) D12115 transcripts which are detectable by amplicon as depicted in sequence name D12115_{—} seg46-47 (SEQ. ID NO: 155) in different normal samples.

FIG. 22 is a histogram showing over expression of the Homo sapiens B-factor, properdin (BF) D12115 transcripts which are detectable by amplicon as depicted in sequence name D12115_seg46-47 (SEQ. ID NO: 155) in cancerous ovarian samples relative to the normal samples.

FIG. 23 shows a graph of cancer and cell-line vs. normal tissue expression for C03950.

FIG. 24 is a histogram showing relative expression of the above-indicated Homo sapiens TNNI3 interacting kinase (TNNI3K) transcripts which are detectable by amplicon as depicted in sequence name C03950_seg44WT (SEQ. ID NO: 233) in heart tissue samples as opposed to other tissues.

FIG. 25 is a histogram showing relative expression of the above-indicated Homo sapiens TNNI3 interacting kinase (TNNI3K) transcripts which are detectable by amplicon as depicted in sequence name C03950_seg51 (SEQ. ID NO: 236) in heart tissue samples as opposed to other tissues.

FIG. 26 is a histogram showing relative expression of the above-indicated Homo sapiens TNNI3 interacting kinase (TNNI3K) transcripts which are detectable by amplicon as depicted in sequence name C03950_seg67F2R2 (SEQ. ID NO: 239) in heart tissue samples as opposed to other tissues.

FIG. 27 shows a graph of cancer and cell-line vs. normal tissue expression for R15601.

FIG. 28 is a histogram showing relative expression of the above-indicated Homo sapiens cardiomyopathy associated 4 (CMYA4) transcripts, which are detectable by amplicon as depicted in sequence name R15601_seg28 (SEQ. ID NO: 272) in heart tissue samples as opposed to other tissues.

FIG. 29 is a histogram showing relative expression of the above-indicated Homo sapiens cardiomyopathy associated 4 (CMYA4) transcripts, which are detectable by amplicon as depicted in sequence name R15601_seg30WT (SEQ. ID NO: 275) in heart tissue samples as opposed to other tissues.

FIG. 30 is a histogram showing relative expression of the above-indicated Homo sapiens myosin, light polypeptide 7, regulatory (MYL7) transcripts, transcripts which are detectable by amplicon as depicted in sequence name T11811_seg14WT (SEQ. ID NO: 311) in heart tissue samples as opposed to other tissues.

FIG. 31 is a histogram showing relative expression of the above-indicated Homo sapiens myosin, light polypeptide 7, regulatory (MYL7) transcripts, transcripts which are detectable by amplicon as depicted in sequence name T11811_seg7-8-9 (SEQ. ID NO: 314) in heart tissue samples as opposed to other tissues.

FIG. 32 is a histogram showing relative expression of the above-indicated Homo sapiens myosin, light polypeptide 7, regulatory (MYL7) transcripts, transcripts which are detectable by amplicon as depicted in sequence name T11811_seg23 (SEQ. ID NO: 317) in heart tissue samples as opposed to other tissues.

### DESCRIPTION OF EMBODIMENTS

The present invention provides polynucleotides, polypeptides, particularly variants of known proteins, and uses thereof, particularly as diagnostic markers.

In some embodiments, the polypeptides and polynucleotides of the present invention are useful as diagnostic markers for certain diseases, and as such the term “marker-detectable” or “variant-detectable” with regard to a disease is to be understood as encompassing use of the described polynucleotides and/or polypeptides for diagnosis.

In some embodiments, certain diseases are associated with differential expression, qualitatively or quantitatively, of the polynucleotides and polypeptides of this invention. Assessment of such expression, in turn, can therefore serve as a marker for a particular disease state, susceptibility to a disease, pathogenesis, etc., including any desired disease-specific event, whose analysis is useful, as will be appreciated by one skilled in the art. In one embodiment, such use as a marker is also referred to herein as the polynucleotides and polypeptides being “variant disease markers”.

The markers of the present invention, alone or in combination, can be used for prognosis, prediction, screening, early diagnosis, staging, therapy selection and treatment monitoring of a marker-detectable disease. For example, optionally and preferably, these markers may be used for staging the disease in patient (for example if the disease features cancer) and/or monitoring the progression of the disease. Furthermore, the markers of the present invention, alone or in combination, can be used for detection of the source of metastasis found in anatomical places other than the originating tissue, again in the example of cancer. Also, one or more of the markers may optionally be used in combination with one or more other disease markers (other than those described herein).

Biomolecular sequences (amino acid and/or nucleic acid sequences) uncovered using the methodology of the present invention and described herein can be efficiently utilized as tissue or pathological markers and/or as drugs or drug targets for treating or preventing a disease.

In some embodiments, these markers are specifically released to the bloodstream under conditions of a particular disease, and/or are otherwise expressed at a much higher level and/or specifically expressed in tissue or cells afflicted with or demonstrating the disease. The measurement of these markers, alone or in combination, in patient samples provides information that the diagnostician can correlate with a probable diagnosis of a particular disease and/or a condition that is indicative of a higher risk for a particular disease.

The present invention provides, in some embodiments, diagnostic assays for a marker-detectable disease and/or an indicative condition, and methods of use of such markers for detection of marker-detectable disease and/or an indicative condition, for example in a sample taken from a subject (patient), which in some embodiments, is a blood sample.

Some embodiments of this invention have been exemplified herein wherein cellular localization was determined according to four different software programs: (i) tmhmm (from Center for Biological Sequence Analysis, Technical University of Denmark DTU, http://www.cbs.dtu.dk/services/TMHMM/TMHMM2.0b.guide.php) or (ii) tmpred (from EMBnet, maintained by the ISREC Bionformatics grdup and the LICR Information Technology Office, Ludwig Institute for Cancer Research, Swiss Institute of Bioinformatics, http://www.ch.embnet.org/software/TMPRED_form.html). for transmembrane region prediction; (iii) signalp_hmm or (iv) signalp_nn (both from Center for Biological Sequence Analysis, Technical University of Denmark DTU, http://www.cbs.dtu.dk/services/SignalP/background/prediction.php) for signal peptide prediction. The terms “signalp_hmm” and “signalp_nn” refer to two modes of operation for the program SignalP: hmm refers to Hidden Markov Model, while nn refers to neural networks. Localization was also determined through manual inspection of known protein localization and/or gene structure, and the use of heuristics by the individual inventor. In some cases for the manual inspection of cellular localization prediction inventors used the ProLoc computational platform [Einat Hazkani-Covo, Erez Levanon, Galit Rotman, Dan Graur and Amit Novik; (2004) “Evolution of multicellularity in metazoa: comparative analysis of the subcellular localization of proteins in Saccharomyces, Drosophila and Caenorhabditis.” Cell Biology International 2004; 28(3):171-8.], which predicts protein localization based on various parameters including, protein domains (e.g., prediction of trans-membranous regions and localization thereof within the protein), pI, protein length, amino acid composition, homology to pre-annotated proteins, recognition of sequence patterns which direct the protein to a certain organelle (such as, nuclear localization signal, NLS, mitochondria localization signal), signal peptide and anchor modeling and using unique domains from Pfam that are specific to a single compartment.

Information is given in the text with regard to SNPs (single nucleotide polymorphisms). A description of the abbreviations is as follows. “T→C”, for example, means that the SNP results in a change at the position given in the table from T to C. Similarly, “M→Q”, for example, means that the SNP has caused a change in the corresponding amino acid sequence, from methionine (M) to glutamine (Q). If, in place of a letter at the right hand side for the nucleotide sequence SNP, there is a space, it indicates that a frame shift has occurred. A frame shift may also be indicated with a hyphen (-). A stop codon is indicated with an asterisk at the right hand side (*). As part of the description of an SNP, a comment may be found in parentheses after the above description of the SNP itself. This comment may include an FTId, which is an identifier to a SwissProt entry that was created with the indicated SNP. An FTId is a unique and stable feature identifier, which allows construction of links directly from position-specific annotation in the feature table to specialized protein-related databases. The FTId is always the last component of a feature in the description field, as follows: FTId=XXX_number, in which XXX is the 3-letter code for the specific feature key, separated by an underscore from a 6-digit number. In the table of the amino acid mutations of the wild type proteins of the selected splice variants of the invention, the header of the first column is “SNP position(s) on amino acid sequence”, representing a position of a known mutation on amino acid sequence. SNPs may optionally be used as diagnostic markers according to the present invention, alone or in combination with one or more other SNPs and/or any other diagnostic marker. Preferred embodiments of the present invention comprise such SNPs, including but not limited to novel SNPs on the known (WT or wild type) protein sequences given below, as well as novel nucleic acid and/or amino acid sequences formed through such SNPs, and/or any SNP on a variant amino acid and/or nucleic acid sequence described herein.

Information given in the text with regard to the Homology to the known proteins was determined by Smith-Waterman version 5.1.2 using special (non default) parameters as follows:
model=sw.model
GAPEXT=0
GAPOP=100.0
   MATRIX=blosum100

Information is given with regard to overexpression of a cluster in cancer based on ESTs. A key to the p values with regard to the analysis of such overexpression is as follows:
Library-based statistics: P-value without including the level of expression in cell-lines (P1)
Library based statistics: P-value including the level of expression in cell-lines (P2)
EST clone statistics: P-value without including the level of expression in cell-lines (SP1)
EST clone statistics: predicted overexpression ratio without including the level of expression in cell-lines (R3)
EST clone statistics: P-value including the level of expression in cell-lines (SP2)
EST clone statistics: predicted overexpression ratio including the level of expression in cell-lines (R4)

Library-based statistics refer to statistics over an entire library, while EST clone statistics refer to expression only for ESTs from a particular tissue or cancer.

Some embodiments of this invention have been exemplified herein wherein overexpression of a cluster in cancer was a determination based on microarray use. As a microarray reference, in the specific segment paragraphs, the unabbreviated tissue name was used as the reference to the type of chip for which expression was measured. There are two types of microarray results: those from microarrays prepared according to a design by the present inventors, for which the microarray fabrication procedure is described in detail in Materials and Experimental Procedures section herein; and those results from microarrays using Affymetrix technology. As a microarray reference, in the specific segment paragraphs, the unabbreviated tissue name was used as the reference to the type of chip for which expression was measured. For microarrays prepared according to a design by the present inventors, the probe name begins with the name of the cluster (gene), followed by an identifying number.

Oligonucleotide microarray results taken from Affymetrix data were from chips available from Affymetrix Inc, Santa Clara, Calif., USA (see for example data regarding the Human Genome U133 (HG-U133) Set at www.affymetrix.com/products/arrays/specific/hgu133.affx; GeneChip Human Genome U133A 2.0 Array at www.affymetrix.com/products/arrays/specific/hgu133av2.affx; and Human Genome U133 Plus 2.0 Array at www.affymetrix.com/products/arrays/specific/hgu133plus.affx). The probe names follow the Affymetrix naming convention. The data is available from NCBI Gene Expression Omnibus (see www.ncbi.nlm.nih.gov/projects/geo/ and Edgar et al, Nucleic Acids Research, 2002, Vol. 30, No. 1 207-210). The dataset (including results) is available from www.ncbi.nlm.nih.gov/geo/query/acc.cgi?acc=GSE1133 for the Series GSE1133 database (published on March 2004); a reference to these results is as follows: Su et al (Proc Natl Acad Sci USA. 2004 Apr. 20; 101(16):6062-7. Epub 2004 Apr. 9).

Oligonucleotide microarray results taken from Affymetrix data were from chips available from Affymetrix Inc, Santa Clara, Calif., USA (see for example data regarding the Human Genome U133 (HG-U133) Set at www.affymetrix.com/products/arrays/specific/hgu133.affx; GeneChip Human Genome U133A 2.0 Array at www.affymetrix.com/products/arrays/specific/hgu133av2.affx; and Human Genome U133 Plus 2.0 Array at www.affymetrix.com/products/arrays/specific/hgu133plus.affx). The probe names follow the Affymetrix naming convention. The data is available from NCBI Gene Expression Omnibus (see www.ncbi.nlm.nih.gov/projects/geo/ and Edgar et al, Nucleic Acids Research, 2002, Vol. 30, No. 1 207-210). The dataset (including results) is available from www.ncbi.nlm.nih.gov/geo/query/acc.cgi?acc=GSE1133 for the Series GSE1133 database (published on March 2004); a reference to these results is as follows: Su et al (Proc Natl Acad Sci USA. 2004 Apr. 20; 101(16):6062-7. Epub 2004 Apr. 9).

The following list of abbreviations for tissues was used in the TAA histograms. The term “TAA” stands for “Tumor Associated Antigen”, and the TAA histograms, given in the text, represent the cancerous tissue expression pattern as predicted by the biomarkers selection engine, as described in detail in examples 1-5 below (the first word is the abbreviation while the second word is the full name):
(“BONE”, “bone”);
(“COL”, “colon”);
(“EPI”, “epithelial”);
(“GEN”, “general”);
(“LIVER”, “liver”);
(“LUN”, “lung”);
(“LYMPH”, “lymph nodes”);
(“MARROW”, “bone marrow”);
(“OVA”, “ovary”);
(“PANCREAS”, “pancreas”);
(“PRO”, “prostate”);
(“STOMACH”, “stomach”);
(“TCELL”, “T cells”);
(“THYROID”, “Thyroid”);
(“MAM”, “breast”);
(“BRAIN”, “brain”);
(“UTERUS”, “uterus”);
(“SKIN”, “skin”);
(“KIDNEY”, “kidney”);
(“MUSCLE”, “muscle”);
(“ADREN”, “adrenal”);
(“HEAD”, “head and neck”);
(“BLADDER”, “bladder”);

It should be noted that the terms “segment”, “seg” and “node” (abbreviated as “N” in the names of nodes) are used interchangeably in reference to nucleic acid sequences of the present invention, they refer to portions of nucleic acid sequences that were shown to have one or more properties as described herein. They are also the building blocks that were used to construct complete nucleic acid sequences as described in greater detail elsewhere herein. Optionally and preferably, they are examples of oligonucleotides which are embodiments of the present invention, for example as amplicons, hybridization units and/or from which primers and/or complementary oligonucleotides may optionally be derived, and/or for any other use.

In some embodiments, the phrase “disease” refers to its commonly understood meaning, and includes, inter alia, any type of pathology and/or damage, including both chronic and acute damage, as well as a progress from acute to chronic damage.

In some embodiments, the phrase “marker” in the context of the present invention refers to a nucleic acid fragment, a peptide, or a polypeptide, which is differentially present in a sample taken from patients (subjects) having one of the herein-described diseases or conditions, as compared to a comparable sample taken from subjects who do not have one the above-described diseases or conditions.

In some embodiments, the term “polypeptide” is to be understood to refer to a molecule comprising from at least 2 to several thousand or more amino acids. The term “polypeptide” is to be understood to include, inter alia, native peptides (either degradation products, synthetically synthesized peptides or recombinant peptides), peptidomimetics, such as peptoids and semipeptoids or peptide analogs, which may comprise, for example, any desirable modification, including, inter alia, modifications rendering the peptides more stable while in a body or more capable of penetrating into cells, or others as will be appreciated by one skilled in the art. Such modifications include, but are not limited to N terminus modification, C terminus modification, peptide bond modification, backbone modifications, residue modification, or others. Inclusion of such peptides within the polypeptides of this invention may produce a polypeptide sharing identity with the polypeptides described herein, for example, those provided in the sequence listing.

In some embodiments, the phrase “differentially present” refers to differences in the quantity or quality of a marker present in a sample taken from patients having one of the herein-described diseases or conditions as compared to a comparable sample taken from patients who do not have one of the herein-described diseases or conditions. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present. Optionally, a relatively low amount of up-regulation may serve as the marker, as described herein. One of ordinary skill in the art could easily determine such relative levels of the markers; further guidance is provided in the description of each individual marker below.

In some embodiments, the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

In some embodiments, the phrase “qualitative” when in reference to differences in expression levels of a polynucleotide, polypeptide or cluster as described herein, refers to the presence versus absence of expression, or in some embodiments, the temporal regulation of expression, or in some embodiments, the timing of expression, or in some embodiments, the variant expressed, or in some embodiments, any post-translational modifications to the expressed molecule, and others, as will be appreciated by one skilled in the art. In some embodiments, the phrase “quantitative” when in reference to differences in expression levels of a polynucleotide, polypeptide or cluster as described herein, refers to absolute differences in quantity of expression, as determined by any means, known in the art, or in other embodiments, relative differences, which may be statistically significant, or in some embodiments, when viewed as a whole or over a prolonged period of time, etc., indicate a trend in terms of differences in expression.

In some embodiments, the term “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the present invention can, in some embodiments, be affected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

In some embodiments, the term “level” refers to expression levels of RNA and/or protein or to DNA copy number of a marker of the present invention.

Typically the level of the marker in a biological sample obtained from the subject is different (i.e., increased or decreased) from the level of the same variant in a similar sample obtained from a healthy individual (examples of biological samples are described herein).

Numerous well known tissue or fluid collection methods can be utilized to collect the biological sample from the subject in order to determine the level of DNA, RNA and/or polypeptide of the variant of interest in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

Determining the level of the same variant in normal tissues of the same origin is preferably effected along-side to detect an elevated expression and/or amplification and/or a decreased expression, of the variant as opposed to the normal tissues.

In some embodiments, the term “test amount” of a marker refers to an amount of a marker in a subject's sample that is consistent with a diagnosis of a particular disease or condition. A test amount can be either in absolute amount (e.g., microgram/m1) or a relative amount (e.g., relative intensity of signals).

In some embodiments, the term “control amount” of a marker can be any amount or a range of amounts to be compared against a test amount of a marker. For example, a control amount of a marker can be the amount of a marker in a patient with a particular disease or condition or a person without such a disease or condition. A control amount can be either in absolute amount (e.g., microgram/ml) or a relative amount (e.g., relative intensity of signals).

In some embodiments, the term “detect” refers to identifying the presence, absence or amount of the object to be detected.

In some embodiments, the term “label” includes any moiety or item detectable by spectroscopic, photo chemical, biochemical, immunochemical, or chemical means. For example, useful labels include ³²P, ³⁵S, fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavadin. The label may be directly or indirectly detectable. Indirect detection can involve the binding of a second label to the first label, directly or indirectly. For example, the label can be the ligand of a binding partner, such as biotin, which is a binding partner for streptavadin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner; Bio/Technology 6:1165 (1988)). Quantitation of the signal is achieved by, e.g., scintillation counting, densitometry, or flow cytometry.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” or “specifically interacts or binds” when referring to a protein or peptide (or other epitope), refers, in some embodiments, to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

In another embodiment, the present invention relates to bridges, tails, heads and/or insertions, and/or analogs, homologs and derivatives of such peptides. Such bridges, tails, heads and/or insertions are described in greater detail below with regard to the Examples.

In some embodiments, the term “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding known protein, while at least a second portion of the variant comprises the tail.

In some embodiments, the term “head” refers to a peptide sequence at the beginning of an amino acid sequence that is unique to a splice variant according to the present invention. Therefore, a splice variant having such a head may optionally be considered as a chimera, in that at least a first portion of the splice variant comprises the head, while at least a second portion is typically highly homologous (often 100% identical) to a portion of the corresponding known protein.

In some embodiments, the term “an edge portion” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between an insertion and a “known protein” portion of a variant.

In some embodiments, a bridge between a tail or a head or a unique insertion, and a “known protein” portion of a variant, comprises at least about 10 amino acids, or in some embodiments at least about 20 amino acids, or in some embodiments at least about 30 amino acids, or in some embodiments at least about 40 amino acids, in which at least one amino acid is from the tail/head/insertion and at least one amino acid is from the “known protein” portion of a variant. In some embodiments, the bridge may comprise any number of amino acids from about 10 to about 40 amino acids (for example, 10, 11, 12, 13 . . . 37, 38, 39, 40 amino acids in length, or any number in between).

It should be noted that a bridge cannot be extended beyond the length of the sequence in either direction, and it should be assumed that every bridge description is to be read in such manner that the bridge length does not extend beyond the sequence itself.

Furthermore, bridges are described with regard to a sliding window in certain contexts below. For example, certain descriptions of the bridges feature the following format: a bridge between two edges (in which a portion of the known protein is not present in the variant) may optionally be described as follows: a bridge portion of CONTIG-NAME_P1 (representing the name of the protein), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise XX (2 amino acids in the center of the bridge, one from each end of the edge), having a structure as follows (numbering according to the sequence of CONTIG-NAME_P1): a sequence starting from any of amino acid numbers 49-x to 49 (for example); and ending at any of amino acid numbers 50+((n-2)−x) (for example), in which x varies from 0 to n-2. In this example, it should also be read as including bridges in which n is any number of amino acids between 10-50 amino acids in length. Furthermore, the bridge polypeptide cannot extend beyond the sequence, so it should be read such that 49-x (for example) is not less than 1, nor 50+((n-2)−x) (for example) greater than the total sequence length.

In another embodiment, this invention provides isolated nucleic acid molecules, which in some embodiments encode for splice variants, having a nucleotide sequence as set forth in any one of the sequences listed herein, being homologous to such sequences, at a percent as described herein, or a sequence complementary thereto. In another embodiment, this invention provides an oligonucleotide of at least about 12 nucleotides, which specifically hybridizes with the nucleic acid molecules of this invention. In another embodiment, this invention provides vectors, cells, liposomes and compositions comprising the isolated nucleic acids or polypeptides of this invention, as appropriate.

In another embodiment, this invention provides a method for detecting the polypeptides of this invention in a biological sample, comprising: contacting a biological sample with an antibody specifically recognizing a splice variant according to the present invention under conditions whereby the antibody specifically interacts with the splice variant in the biological sample but do not recognize known corresponding proteins (wherein the known protein is discussed with regard to its splice variant(s) in the Examples below), and detecting said interaction; wherein the presence of an interaction correlates with the presence of a splice variant in the biological sample.

In another embodiment, this invention provides a method for detecting a polynucleotide of this invention in a biological sample, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of a the polynucleotide in the biological sample.

In some embodiments of the present invention, the polypeptides/polynucleotides described herein are non-limiting examples of markers for diagnosing marker-detectable disease and/or an indicative condition. Each polypeptide/polynucleotide marker of the present invention can be used alone or in combination, for various uses, including but not limited to, prognosis, prediction, screening, early diagnosis, determination of progression, therapy selection and treatment monitoring of marker-detectable disease and/or an indicative condition, including a transition from an indicative condition to marker-detectable disease.

According to some embodiments of the present invention, any marker according to the present invention may optionally be used alone or combination. Such a combination may optionally comprise a plurality of markers described herein, optionally including any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker. With regard to such a ratio between any marker described herein (or a combination thereof) and a known marker, more preferably the known marker comprises the “known protein” as described in greater detail below with regard to each cluster or gene.

In some embodiments of the present invention, there are provided of methods, uses, devices and assays for the diagnosis of a disease or condition. Optionally a plurality of biomarkers (or markers) may be used with the present invention. The plurality of markers may optionally include a plurality of markers described herein, and/or one or more known markers. The plurality of markers is preferably then correlated with the disease or condition. For example, such correlating may optionally comprise determining the concentration of each of the plurality of markers, and individually comparing each marker concentration to a threshold level. Optionally, if the marker concentration is above or below the threshold level (depending upon the marker and/or the diagnostic test being performed), the marker concentration correlates with the disease or condition. Optionally and preferably, a plurality of marker concentrations correlates with the disease or condition.

Alternatively, such correlating may optionally comprise determining the concentration of each of the plurality of markers, calculating a single index value based on the concentration of each of the plurality of markers, and comparing the index value to a threshold level.

Also alternatively, such correlating may optionally comprise determining a temporal change in at least one of the markers, and wherein the temporal change is used in the correlating step.

Also alternatively, such correlating may optionally comprise determining whether at least “X” number of the plurality of markers has a concentration outside of a predetermined range and/or above or below a threshold (as described above). The value of “X” may optionally be one marker, a plurality of markers or all of the markers; alternatively or additionally, rather than including any marker in the count for “X”, one or more specific markers of the plurality of markers may optionally be required to correlate with the disease or condition (according to a range and/or threshold).

Also alternatively, such correlating may optionally comprise determining whether a ratio of marker concentrations for two markers is outside a range and/or above or below a threshold. Optionally, if the ratio is above or below the threshold level and/or outside a range, the ratio correlates with the disease or condition.

Optionally, a combination of two or more these correlations may be used with a single panel and/or for correlating between a plurality of panels.

Optionally, the method distinguishes a disease or condition with a sensitivity of at least 70% at a specificity of at least 85% when compared to normal subjects. As used herein, sensitivity relates to the number of positive (diseased) samples detected out of the total number of positive samples present; specificity relates to the number of true negative (non-diseased) samples detected out of the total number of negative samples present. Preferably, the method distinguishes a disease or condition with a sensitivity of at least 80% at a specificity of at least 90% when compared to normal subjects. More preferably, the method distinguishes a disease or condition with a sensitivity of at least 90% at a specificity of at least 90% when compared to normal subjects. Also more preferably, the method distinguishes a disease or condition with a sensitivity of at least 70% at a specificity of at least 85% when compared to subjects exhibiting symptoms that mimic disease or condition symptoms.

A marker panel may be analyzed in a number of fashions well known to those of skill in the art. For example, each member of a panel may be compared to a “normal” value, or a value indicating a particular outcome. A particular diagnosis/prognosis may depend upon the comparison of each marker to this value; alternatively, if only a subset of markers is outside of a normal range, this subset may be indicative of a particular diagnosis/prognosis. The skilled artisan will also understand that diagnostic markers, differential diagnostic markers, prognostic markers, time of onset markers, disease or condition differentiating markers, etc., may be combined in a single assay or device. Markers may also be commonly used for multiple purposes by, for example, applying a different threshold or a different weighting factor to the marker for the different purpose(s).

In one embodiment, the panels comprise markers for the following purposes: diagnosis of a disease; diagnosis of disease and indication if the disease is in an acute phase and/or if an acute attack of the disease has occurred; diagnosis of disease and indication if the disease is in a non-acute phase and/or if a non-acute attack of the disease has occurred; indication whether a combination of acute and non-acute phases or attacks has occurred; diagnosis of a disease and prognosis of a subsequent adverse outcome; diagnosis of a disease and prognosis of a subsequent acute or non-acute phase or attack; disease progression (for example for cancer, such progression may include for example occurrence or recurrence of metastasis).

The above diagnoses may also optionally include differential diagnosis of the disease to distinguish it from other diseases, including those diseases that may feature one or more similar or identical symptoms.

In certain embodiments, one or more diagnostic or prognostic indicators are correlated to a condition or disease by merely the presence or absence of the indicator(s). In other embodiments, threshold level(s) of a diagnostic or prognostic indicator(s) can be established, and the level of the indicator(s) in a patient sample can simply be compared to the threshold level(s). The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations, and/or by comparison of results from a subject before, during and/or after treatment. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art (see for example Hanley et al., Radiology 143: 29-36 (1982), incorporated by reference as if fully set forth herein).

One or more markers may lack diagnostic or prognostic value when considered alone, but when used as part of a panel, such markers may be of great value in determining a particular diagnosis/prognosis. In some embodiments, particular thresholds for one or more markers in a panel are not relied upon to determine if a profile of marker levels obtained from a subject are indicative of a particular diagnosis/prognosis. Rather, the present invention may utilize an evaluation of the entire marker profile by plotting ROC curves for the sensitivity of a particular panel of markers versus 1-(specificity) for the panel at various cutoffs. In these methods, a profile of marker measurements from a subject is considered together to provide a global probability (expressed either as a numeric score or as a percentage risk) that an individual has had a disease, is at risk for developing such a disease, optionally the type of disease which the individual has had or is at risk for, and so forth etc. In such embodiments, an increase in a certain subset of markers may be sufficient to indicate a particular diagnosis/prognosis in one patient, while an increase in a different subset of markers may be sufficient to indicate the same or a different diagnosis/prognosis in another patient. Weighting factors may also be applied to one or more markers in a panel, for example, when a marker is of particularly high utility in identifying a particular diagnosis/prognosis, it may be weighted so that at a given level it alone is sufficient to signal a positive result. Likewise, a weighting factor may provide that no given level of a particular marker is sufficient to signal a positive result, but only signals a result when another marker also contributes to the analysis.

In some embodiments, markers and/or marker panels are selected to exhibit at least 70% sensitivity, more preferably at least 80% sensitivity, even more preferably at least 85% sensitivity, still more preferably at least 90% sensitivity, and most preferably at least 95% sensitivity, combined with at least 70% specificity, more preferably at least 80% specificity, even more preferably at least 85% specificity, still more preferably at least 90% specificity, and most preferably at least 95% specificity. In some embodiments, both the sensitivity and specificity are at least 75%, more preferably at least 80%, even more preferably at least 85%, still more preferably at least 90%, and most preferably at least 95%. Sensitivity and/or specificity may optionally be determined as described above, with regard to the construction of ROC graphs and so forth, for example.

According to some embodiments of the present invention, individual markers and/or combinations (panels) of markers may optionally be used for diagnosis of time of onset of a disease or condition. Such diagnosis may optionally be useful for a wide variety of conditions, preferably including those conditions with an abrupt onset.

The phrase “determining the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is more likely to occur than not. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. For example, in individuals not exhibiting the condition, the chance of a given outcome may be about 3%. In some embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−1%.

The skilled artisan will understand that associating a prognostic indicator with a predisposition to an adverse outcome is a statistical analysis. For example, a marker level of greater than 80 pg/mL may signal that a patient is more likely to suffer from an adverse outcome than patients with a level less than or equal to 80 pg/mL, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value. See, e.g., Dowdy and Wearden, Statistics for Research, John Wiley & Sons,

New York, 1983. In one embodiment the confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. Exemplary statistical tests for associating a prognostic indicator with a predisposition to an adverse outcome are described hereinafter.

In other embodiments, a threshold degree of change in the level of a prognostic or diagnostic indicator can be established, and the degree of change in the level of the indicator in a patient sample can simply be compared to the threshold degree of change in the level. A preferred threshold change in the level for markers of the invention is about 5%, about 10%, about 15%, about 20%, about 25%, about 30%, about 50%, about 75%, about 100%, and about 150%. The term “about” in this context refers to +/−10%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

Exemplary, non-limiting methods and systems for identification of suitable biomarkers for marker panels are now described. Methods and systems for the identification of one or more markers for the diagnosis, and in particular for the differential diagnosis, of disease have been described previously. Suitable methods for identifying markers useful for the diagnosis of disease states are described in detail in U.S. patent application no. 2004-0126767, entitled METHOD AND SYSTEM FOR DISEASE DETECTION USING MARKER COMBINATIONS, filed Dec. 27, 2002, hereby incorporated by reference in its entirety as if fully set forth herein. One skilled in the art will also recognize that univariate analysis of markers can be performed and the data from the univariate analyses of multiple markers can be combined to form panels of markers to differentiate different disease conditions.

In developing a panel of markers useful in diagnosis, data for a number of potential markers may be obtained from a group of subjects by testing for the presence or level of certain markers. The group of subjects is divided into two sets, and preferably the first set and the second set each have an approximately equal number of subjects. The first set includes subjects who have been confirmed as having a disease or, more generally, being in a first condition state. For example, this first set of patients may be those that have recently had a disease and/or a particular type of the disease. The confirmation of this condition state may be made through more rigorous and/or expensive testing, preferably according to a previously defined diagnostic standard. Hereinafter, subjects in this first set will be referred to as “diseased”.

The second set of subjects is simply those who do not fall within the first set. Subjects in this second set may be “non-diseased;” that is, normal subjects. Alternatively, subjects in this second set may be selected to exhibit one symptom or a constellation of symptoms that mimic those symptoms exhibited by the “diseased” subjects.

The data obtained from subjects in these sets includes levels of a plurality of markers. Preferably, data for the same set of markers is available for each patient. This set of markers may include all candidate markers which may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a marker often is incapable of definitively identifying a patient as either diseased or non-diseased. For example, if a patient is measured as having a marker level that falls within the overlapping region, the results of the test will be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve as described above.

As discussed above, the measurement of the level of a single marker may have limited usefulness. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In an embodiment method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients is measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In an embodiment method, acceptable weighting coefficients may range between zero and one, and an initial weighting coefficient for each marker may be assigned as 0.5. In one embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In some embodiments, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In one embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. In order to develop lower-cost panels which require the measurement of fewer marker levels, certain markers may be eliminated from the panel. In this regard, the effective contribution of each marker in the panel may be determined to identify the relative importance of the markers. In one embodiment, the weighting coefficients resulting from the optimization process may be used to determine the relative importance of each marker. The markers with the lowest coefficients may be eliminated.

Individual panel response values may also be used as markers in the methods described herein. For example, a panel may be constructed from a plurality of markers, and each marker of the panel may be described by a function and a weighting factor to be applied to that marker (as determined by the methods described above). Each individual marker level is determined for a sample to be tested, and that level is applied to the predetermined function and weighting factor for that particular marker to arrive at a sample value for that marker. The sample values for each marker are added together to arrive at the panel response for that particular sample to be tested. For a “diseased” and “non-diseased” group of patients, the resulting panel responses may be treated as if they were just levels of another disease marker.

Measures of test accuracy may be obtained as described in Fischer et al., Intensive Care Med. 29: 1043-51, 2003 (hereby incorporated by reference as if fully set forth herein), and used to determine the effectiveness of a given marker or panel of markers. These measures include sensitivity and specificity, predictive values, likelihood ratios, diagnostic odds ratios, and ROC curve areas. As discussed above, suitable tests may exhibit one or more of the following results on these various measures: at least 75% sensitivity, combined with at least 75% specificity; ROC curve area of at least 0.7, more preferably at least 0.8, even more preferably at least 0.9, and most preferably at least 0.95; and/or a positive likelihood ratio (calculated as sensitivity/(1-specificity)) of at least 5, more preferably at least 10, and most preferably at least 20, and a negative likelihood ratio (calculated as (1-sensitivity)/specificity) of less than or equal to 0.3, more preferably less than or equal to 0.2, and most preferably less than or equal to 0.1.

According to embodiments of the present invention, a splice variant protein or a fragment thereof, or a splice variant nucleic acid sequence or a fragment thereof, may be featured as a biomarker for detecting marker-detectable disease and/or an indicative condition, such that a biomarker may optionally comprise any of the above.

According to still other embodiments, the present invention optionally and preferably encompasses any amino acid sequence or fragment thereof encoded by a nucleic acid sequence corresponding to a splice variant protein as described herein. Any oligopeptide or peptide relating to such an amino acid sequence or fragment thereof may optionally also (additionally or alternatively) be used as a biomarker, including but not limited to the unique amino acid sequences of these proteins that are depicted as tails, heads, insertions, edges or bridges. The present invention also optionally encompasses antibodies capable of recognizing, and/or being elicited by, such oligopeptides or peptides.

The present invention also optionally and preferably encompasses any nucleic acid sequence or fragment thereof, or amino acid sequence or fragment thereof, corresponding to a splice variant of the present invention as described above, optionally for any application.

Non-limiting examples of methods or assays are described below.

The present invention also relates to kits based upon such diagnostic methods or assays.

### Nucleic Acid Sequences and Oligonucleotides

Various embodiments of the present invention encompass nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or artificially induced, either randomly or in a targeted fashion.

The present invention encompasses nucleic acid sequences described herein; fragments thereof, sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth below], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

A “nucleic acid fragment” or an “oligonucleotide” or a “polynucleotide” are used herein interchangeably to refer to a polymer of nucleic acids. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Preferred embodiments of the present invention encompass oligonucleotide probes.

An example of an oligonucleotide probe which can be utilized by the present invention is a single stranded polynucleotide which includes a sequence complementary to the unique sequence region of any variant according to the present invention, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Alternatively, an oligonucleotide probe of the present invention can be designed to hybridize with a nucleic acid sequence encompassed by any of the above nucleic acid sequences, particularly the portions specified above, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases. Preferably, the oligonucleotide of the present invention features at least 17, at least 18, at least 19, at least 20, at least 22, at least 25, at least 30 or at least 40, bases specifically hybridizable with the biomarkers of the present invention.

The oligonucleotides of the present invention may comprise heterocylic nucleosides consisting of purines and the pyrimidines bases, bonded in a 3′ to 5′ phosphodiester linkage.

Preferably used oligonucleotides are those modified at one or more of the backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos.: 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkyiphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623, 070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic includes peptide nucleic acid (PNA). United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention, are disclosed in U.S. Pat. No: 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases particularly useful for increasing the binding affinity of the oligomeric compounds of the invention include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No: 6,303,374.

It is not necessary for all positions in a given oligonucleotide molecule to be uniformly modified, and in fact more than one of the aforementioned modifications may be incorporated in a single compound or even at a single nucleoside within an oligonucleotide.

It will be appreciated that oligonucleotides of the present invention may include further modifications for more efficient use as diagnostic agents and/or to increase bioavailability, therapeutic efficacy and reduce cytotoxicity.

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific, lymphoid specific promoters [Calame at al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Variant Recombinant Expression Vectors and Host Cells

Another aspect of the invention pertains to vectors, preferably expression vectors, containing a nucleic acid encoding a variant protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., variant proteins, mutant forms of variant proteins, fusion proteins, etc.).

The recombinant expression vectors of the invention can be designed for production of variant proteins in prokaryotic or eukaryotic cells. For example, variant proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, to the amino or carboxyl terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, PreScission, TEV and enterokinase. Typical fusion expression vectors include pGEX (Pharmacia Biotech Inc.; Smith and Johnson, 1988. Gene 67: 31-40), pMAL (New England Biolabs, Beverly, Mass.) and pRIT5 (Pharmacia, Piscataway, N.J.) and pTrcHis (Invitrogen Life Technologies) that fuse glutathione S-transferase (GST), maltose E binding protein, protein A or 6xHis, respectively, to the target recombinant protein.

Examples of suitable inducible non-fusion E. coli expression vectors include pTrc (Amrann et al., (1988) Gene 69:301-315).

One strategy to maximize recombinant protein expression in E. coli is to express the protein in host bacteria with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques. Another optional strategy to solve codon bias is by using BL21-codon plus bacterial strains (Invitrogen) or Rosetta bacterial strain (Novagen), as these strains contain extra copies of rare E. coli tRNA genes.

In another embodiment, the expression vector encoding for the variant protein is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, variant protein can be produced in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195), pIRESpuro (Clontech), pUB6 (Invitrogen), pCEP4 (Invitrogen) pREP4 (Invitrogen), pcDNA3 (Invitrogen). When used in mammalian cells, the expression vector's control functions are often provided by viral regulatory elements. For example, commonly used promoters are derived from polyoma, adenovirus 2, cytomegalovirus, Rous Sarcoma Virus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to mRNA encoding for variant protein. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, variant protein can be produced in bacterial cells such as E. coli, insect cells, yeast or mammalian cells (such as Chinese hamster ovary cells (CHO) or COS or 293 cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin, puromycin, blasticidin and methotrexate. Nucleic acids encoding a selectable marker can be introduced into a host cell on the same vector as that encoding variant protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) variant protein. Accordingly, the invention further provides methods for producing variant protein using the host cells of the invention. In one embodiment, the method comprises culturing the host cell of the present invention (into which a recombinant expression vector encoding variant protein has been introduced) in a suitable medium such that variant protein is produced. In another embodiment, the method further comprises isolating variant protein from the medium or the host cell.

For efficient production of the protein, it is preferable to place the nucleotide sequences encoding the variant protein under the control of expression control sequences optimized for expression in a desired host. For example, the sequences may include optimized transcriptional and/or translational regulatory sequences (such as altered Kozak sequences).

### Hybridization Assays

Detection of a nucleic acid of interest in a biological sample may optionally be effected by hybridization-based assays using an oligonucleotide probe (non-limiting examples of probes according to the present invention were previously described).

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Hybridization based assays which allow the detection of a variant of interest (i.e., DNA or RNA) in a biological sample rely on the use of oligonucleotides which can be 10, 15, 20, or 30 to 100 nucleotides long preferably from 10 to 50, more preferably from 40 to 50 nucleotides long.

Thus, the isolated polynucleotides (oligonucleotides) of the present invention are preferably hybridizable with any of the herein described nucleic acid sequences under moderate to stringent hybridization conditions.

Moderate to stringent hybridization conditions are characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1 SDS and 5×10⁶ cpm ³²P labeled probe, at 65° C., with a final wash solution of 1×SSC and 0.1% SDS and final wash at 50° C.

More generally, hybridization of short nucleic acids (below 200 by in length, e.g. 17-40 by in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5 SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of radioactive labels include 3H, 14C, 32P, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cyt, Cy3, Cy3.5, Cy5, Cy5.5, Cy7, FluorX (Amersham) and others [e.g., Kricka et al: (1992), Academic Press San Diego, Calif.] can be attached to the oligonucleotides.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Although the present invention is not specifically dependent on the use of a label for the detection of a particular nucleic acid sequence, such a label might be benefic,ial, by increasing the sensitivity of the detection. Furthermore, it enables automation. Probes can be labeled according to numerous well known methods.

As commonly known, radioactive nucleotides can be incorporated into probes of the invention by several methods. Non-limiting examples of radioactive labels include ³H, ¹⁴C, ³²P, and ³⁵S.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA), and preferably of DNA.

### NAT Assays

Detection of a nucleic acid of interest in a biological sample may also optionally be effected by NAT-based assays, which involve nucleic acid amplification technology, such as PCR for example (or variations thereof such as real-time PCR for example).

As used herein, a “primer” defines an oligonucleotide which is capable of annealing to (hybridizing with) a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14 Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

It will be appreciated that antisense oligonucleotides may be employed to quantify expression of a splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity.

The polymerase chain reaction and other nucleic acid amplification reactions are well known in the art (various non-limiting examples of these reactions are described in greater detail below). The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., id between 3° C. and 0° C.

Polymerase Chain Reaction (PCR): The polymerase chain reaction (PCR), as described in

U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation and ligation amplify a short segment of DNA. LCR has also been used in combination with PCR to achieve enhanced detection of single-base changes: see for example Segev, PCT Publication No. W09001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR/NASBA): The self-sustained sequence replication reaction (3SR) is a transcription-based in vitro amplification system that can exponentially amplify RNA sequences at a uniform temperature. The amplified RNA can then be utilized for mutation detection. In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo-and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In this method, a probe which recognizes the sequence of interest is attached to the replica table RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55 degrees C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (1+X)n =y, where “X” is the mean efficiency (percent copied in each cycle), “n” is the number of cycles, and “y” is the overall efficiency, or yield of the reaction. If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.8520₂₀, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Additional NAT tests are Fluorescence In Situ Hybridization (FISH) and Comparative Genomic Hybridization (CGH). Fluorescence In Situ Hybridization (FISH)—The test uses fluorescent single-stranded DNA probes which are complementary to the DNA sequences that are under examination (genes or chromosomes). These probes hybridize with the complementary DNA and allow the identification of the chromosomal location of genomic sequences of DNA.

Comparative Genomic Hybridization (CGH)—allows a comprehensive analysis of multiple DNA gains and losses in entire genomes. Genomic DNA from the tissue to be investigated and a reference DNA are differentially labeled and simultaneously hybridized in situ to normal metaphase chromosomes. Variations in signal intensities are indicative of differences in the genomic content of the tissue under investigation.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The detection of at least one sequence change according to various preferred embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis or Dideoxy fingerprinting (ddF).

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes has 4 to 6 base-pair recognition sequences, and cleaves too frequently for many large-scale DNA manipulations. Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered. Recently, endonucleases encoded by group I introns have been discovered that might have greater than 12 base-pair specificity, but again, these are few in number.

Allele specific oligonucleotide (ASO): If the change is not in a recognition sequence, then allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations. The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes and gsp/gip oncogenes. Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE. Attaching a GC clamp to one primer is critical to ensure that the amplified sequence has a low dissociation temperature. Modifications of the technique have been developed, using temperature gradients, and the method can be also applied to RNA:RNA duplexes.

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient. TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations.

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations. The ddF technique combines components of

Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

According to a presently preferred embodiment of the present invention the step of searching for any of the nucleic acid sequences described here, in tumor cells or in cells derived from a cancer patient is effected by any suitable technique, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

Detection may also optionally be performed with a chip or other such device. The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for a disease and/or pathological condition both rapidly and easily.

### Amino Acid Sequences and Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The present invention also encompasses polypeptides encoded by the polynucleotide sequences of the present invention, as well as polypeptides according to the amino acid sequences described herein. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth below, as can be determined using BlastP software of the National Center of Biotechnology Information (NCBI) using default parameters, optionally and preferably including the following: filtering on (this option filters repetitive or low-complexity sequences from the query using the Seg (protein) program), scoring matrix is BLOSUM62 for proteins, word size is 3, E value is 10, gap costs are 11, 1 (initialization and extension), and number of alignments shown is 50. Preferably, nucleic acid sequence homology/identity is determined by using BlastN software of the National Center of Biotechnology Information (NCBI) using default parameters, which preferably include using the DUST filter program, and also preferably include having an E value of 10, filtering low complexity sequences and a word size of 11. Finally, the present invention also encompasses fragments of the above described polypeptides and polypeptides having mutations, such as deletions, insertions or substitutions of one or more amino acids, either naturally occurring or artificially induced, either randomly or in a targeted fashion.

It will be appreciated that peptides identified according the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2—NH, CH2—S, CH2—S═O, O═C—NH, CH2—O, CH2—CH2, S═C—NH, CH═CH or CF═CH, backbone modifications, and residue modification. Methods for preparing peptidomimetic compounds are well known in the art and are specified. Further details in this respect are provided hereinunder.

Peptide bonds (—CO—NH—) within the peptide may be substituted, for example, by N-methylated bonds (—N(CH3)—CO—), ester bonds (—C(R)H—C—O—O—C(R)—N—), ketomethylen bonds (—CO—CH2—), α-aza bonds (—NH—N(R)—CO—), wherein R is any alkyl, e.g., methyl, carba bonds (—CH2—NH—), hydroxyethylene bonds (—CH(OH)—CH2—), thioamide bonds (—CS—NH—), olefmic double bonds (—CH═CH—), retro amide bonds (—NH—CO—), peptide derivatives (—N(R)—CH2—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc.).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids. Non-conventional or modified amino acids can be incorporated in the polypeptides of this invention as well, as will be known to one skilled in the art.

Since the peptides of the present invention are preferably utilized in diagnostics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclization does not severely interfere with peptide characteristics, cyclic forms of the peptide can also be utilized.

The peptides of present invention can be biochemically synthesized such as by using standard solid phase techniques. These methods include exclusive solid phase synthesis well known in the art, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Synthetic peptides can be purified by preparative high performance liquid chromatography and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463 and also as described above.

### Antibodies:

“Antibody” refers to a polypeptide ligand that is preferably substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fe” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)2, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Monoclonal antibody development may optionally be performed according to any method that is known in the art. The method described below is provided for the purposes of description only and is not meant to be limiting in any way.

### Antibody Engineering in Phage Display Libraries:

Antibodies of this invention may be prepared through the use of phage display libraries, as is known in the art, for example, as described in PCT Application No. WO 94/18219, U.S. Pat. No. 6,096,551, both of which are hereby fully incorporated by reference, The method involves inducing mutagenesis in a complementarity determining region (CDR) of an immunoglobulin light chain gene for the purpose of producing light chain gene libraries for use in combination with heavy chain genes and gene libraries to produce antibody libraries of diverse and novel immuno-specificities. The method comprises amplifying a CDR portion of an immunoglobulin light chain gene by polymerase chain reaction (PCR) using a PCR primer oligonucleotide. The resultant gene portions are inserted into phagemids for production of a phage display library, wherein the engineered light chains are displayed by the phages, for example for testing their binding specificity.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using Papain produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (1972)]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. A scFv antibody fragment is an engineered antibody derivative that includes heavy- and light chain variable regions joined by a peptide linker. The minimal size of antibody molecules are those that still comprise the complete antigen binding site. ScFv antibody fragments are potentially more effective than unmodified IgG antibodies. The reduced size of 27-30 kDa permits them to penetrate tissues and solid tumors more readily. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)]. Optionally, there may be 1, 2 or 3 CDRs of different chains, but preferably there are 3 CDRs of 1 chain. The chain could be the heavy or the light chain.

Humanized forms of non-human (e.g., murine) antibodies, are chimeric molecules of immunoglobulins, immunoglobulin chains or fragments thereof (such as Fv, Fab, Fab′, F(ab′) or other antigen-binding subsequences of antibodies) which contain minimal sequence derived from non-human immunoglobulin, or fragments thereof may comprise the antibodies of this invention. Humanized antibodies are well known in the art. Methods for humanizing non-human antibodies are well known in the art, for example, as described in Winter and co-workers [Jones et al., Nature, 321:522-525 (1986); Riechmann et al., Nature 332:323-327 (1988); Verhoeyen et al., Science, 239:1534-1536 (1988)], U.S. Pat. No. 4,816,567, Hoogenboom and Winter, J. Mol. Biol., 227:381 (1991); Marks et al., J. Mol. Biol., 222:581 (1991), Cole et al., Monoclonal Antibodies and Cancer Therapy, Alan R. Liss, p. 77 (1985), Boerner et al., J. Immunol., 147(1):86-95 (1991), U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995), all of which are incorporated herein by reference.

Preferably, the antibody of this aspect of the present invention specifically binds at least one epitope of the polypeptide variants of the present invention. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

Optionally, a unique epitope may be created in a variant due to a change in one or more post-translational modifications, including but not limited to glycosylation and/or phosphorylation, as described below. Such a change may also cause a new epitope to be created, for example through removal of glycosylation at a particular site.

An epitope according to the present invention may also optionally comprise part or all of a unique sequence portion of a variant according to the present invention in combination with at least one other portion of the variant which is not contiguous to the unique sequence portion in the linear polypeptide itself, yet which are able to form an epitope in combination. One or more uniqUe sequence portions may optionally combine with one or more other non-contiguous portions of the variant (including a portion which may have high homology to a portion of the known protein) to form an epitope.

### Immunoassays

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

To prepare an antibody that specifically binds to a marker, purified protein markers can be used. Antibodies that specifically bind to a protein marker can be prepared using any suitable methods known in the art.

After the antibody is provided, a marker can be detected and/or quantified using any of a number of well recognized immunological binding assays. Useful assays include, for example, an enzyme immune assay (EIA) such as enzyme-linked immunosorbent assay (ELISA), a radioimmune assay (RIA), a Western blot assay, or a slot blot assay see, e.g., U.S. Pat. Nos. 4,366,241; 4,376,110; 4,517,288; and 4,837,168). Generally, a sample obtained from a subject can be contacted with the antibody that specifically binds the marker.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead. Antibodies can also be attached to a solid support.

After incubating the sample with antibodies, the mixture is washed and the antibody-marker complex formed can be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal.

Preferably used are antibodies which specifically interact with the polypeptides of the present invention and not with wild type proteins or other isoforms thereof, for example. Such antibodies are directed, for example, to the unique sequence portions of the polypeptide variants of the present invention, including but not limited to bridges, heads, tails and insertions described in greater detail below. Preferred embodiments of antibodies according to the present invention are described in greater detail with regard to the section entitled “Antibodies”.

Radio-immunoassay (RIA): In one version, this method involves precipitation of the desired substrate and in the methods detailed hereinbelow, with a specific antibody and radiolabelled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, colorimetric reaction or chemiluminescence. This method allows both quantitation of an amount of Substrate and determination of its identity by a relative position on the membrane which is indicative of a Migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a colorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

### Radio-Imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference as if fully set forth herein.

### Display Libraries

According to still another aspect of the present invention there is provided a display library comprising a plurality of display vehicles (such as phages, viruses or bacteria) each displaying at least 6, at least 7, at least 8, at least 9, at least 10, 10-15, 12-17, 15-20, 15-30 or 20-50 consecutive amino acids derived from the polypeptide sequences of the present invention.

Methods of constructing such display libraries are well known in the art. Such methods are described in, for example, Young A C, et al., “The three-dimensional structures of a polysaccharide binding antibody to Cryptococcus neoformans and its complex with a peptide from a phage display library: implications for the identification of peptide mimotopes” J Mol Biol 1997 Dec. 12; 274(4):622-34; Giebel LB et al. “Screening of cyclic peptide phage libraries identifies ligands that bind streptavidin with high affinities” Biochemistry 1995 Nov. 28 ;34(47):15430-5; Davies E L et al., “Selection of specific phage-display antibodies using libraries derived from chicken immunoglobulin genes” J Immunol Methods 1995 Oct. 12; 186(1):125-35; Jones C R T al. “Current trends in molecular recognition and bioseparation” J Chromatogr A 1995 Jul. 14; 707(1):3-22; Deng S J et al. “Basis for selection of improved carbohydrate-binding single-chain antibodies from synthetic gene libraries” Proc Natl Acad Sci U S A 1995 May 23; 92(11):4992-6; and Deng S J et al. “Selection of antibody single-chain variable fragments with improved carbohydrate binding by phage display” J Biol Chem 1994 Apr. 1; 269(13):9533-8, which are incorporated herein by reference.

### Theranostics:

The term theranostics describes the use of diagnostic testing to diagnose the disease, choose the correct treatment regime according to the results of diagnostic testing and/or monitor the patient response to therapy according to the results of diagnostic testing. Theranostic tests can be used to select patients for treatments that are particularly likely to benefit them and unlikely to produce side-effects. They can also provide an early and objective indication of treatment efficacy in individual patients, so that (if necessary) the treatment can be altered with a minimum of delay. For example: DAKO and Genentech together created HercepTest and Herceptin (trastuzumab) for the treatment of breast cancer, the first theranostic test approved simultaneously with a new therapeutic drug. In addition to HercepTest (which is an immunohistochemical test), other theranostic tests are in development which use traditional clinical chemistry, immunoassay, cell-based technologies and nucleic acid tests. PPGx's recently launched TPMT (thiopurine S-methyltransferase) test, which is enabling doctors to identify patients at risk for potentially fatal adverse reactions to 6-mercaptopurine, an agent used in the treatment of leukemia. Also, Nova Molecular pioneered SNP genotyping of the apolipoprotein E gene to predict Alzheimer's disease patients' responses to cholinomimetic therapies and it is now widely used in clinical trials of new drugs for this indication. Thus, the field of theranostics represents the intersection of diagnostic testing information that predicts the response of a patient to a treatment with the selection of the appropriate treatment for that particular patient.

### Surrogate Markers:

A surrogate marker is a marker, that is detectable in a laboratory and/or according to a physical sign or symptom on the patient, and that is used in therapeutic trials as a substitute for a clinically meaningful endpoint. The surrogate marker is a direct measure of how a patient feels, functions, or survives which is expected to predict the effect of the therapy. The need for surrogate markers mainly arises when such markers can be measured earlier, more conveniently, or more frequently than the endpoints of interest in terms of the effect of a treatment on a patient, which are referred to as the clinical endpoints. Ideally, a surrogate marker should be biologically plausible, predictive of disease progression and measurable by standardized assays (including but not limited to traditional clinical chemistry, immunoassay, cell-based technologies, nucleic acid tests and imaging modalities).

Surrogate endpoints were used first mainly in the cardiovascular area. For example, antihypertensive drugs have been approved based on their effectiveness in lowering blood pressure. Similarly, in the past, cholesterol-lowering agents have been approved based on their ability to decrease serum cholesterol, not on the direct evidence that they decrease mortality from atherosclerotic heart disease. The measurement of cholesterol levels is now an accepted surrogate marker of atherosclerosis. In addition, currently two commonly used surrogate markers in HIV studies are CD4+ T cell counts and quantitative plasma HIV RNA (viral load). In some embodiments of this invention, the polypeptide/polynucleotide expression pattern may serve as a surrogate marker for a particular disease, as will be appreciated by one skilled in the art.

### Monoclonal Antibody Therapy:

In some embodiments, monoclonal antibodies are useful for the identification of cancer cells. In some embodiments, monoclonal antibody therapy is a form of passive immunotherapy useful in cancer treatment. Such antibodies may comprise naked monoclonal antibodies or conjugated monoclonal antibodies—joined to a chemotherapy drug, radioactive particle, or a toxin (a substance that poisons cells). In some embodiments, the former is directly cytotoxic to the target (cancer) cell, or in another embodiment, stimulates or otherwise participates in an immune response ultimately resulting in the lysis of the target cell.

In some embodiments, the conjugated monoclonal antibodies are joined to drugs, toxins, or radioactive atoms. They are used as delivery vehicles to take those substances directly to the cancer cells. The MAb acts as a homing device, circulating in the body until it finds a cancer cell with a matching antigen. It delivers the toxic substance to where it is needed most, minimizing damage to normal cells in other parts of the body. Conjugated MAbs are also sometimes referred to as “tagged,” “labeled,” or “loaded” antibodies. MAbs with chemotherapy drugs attached are generally referred to as chemolabeled. MAbs with radioactive particles attached are referred to as radiolabeled, and this type of therapy is known as radioimmunotherapy (RID. MAbs attached to toxins are called immunotoxins.

An illustrative, non-limiting example is provided herein of a method of treatment of a patient with an antibody to a variant as described herein, such that the variant is a target of the antibody. A patient with breast cancer is treated with a radiolabeled humanized antibody against an appropriate breast cancer target as described herein. The patient is optionally treated with a dosage of labeled antibody ranging from 10 to 30 mCi. Of course any type of therapeutic label may optionally be used.

The following sections relate to Candidate Marker Examples. It should be noted that Table numbering is restarted within each Example, which starts with the words “Description for Cluster”.

### Candidate Marker Examples Section

This Section relates to Examples of sequences according to the present invention, including illustrative methods of selection thereof with regard to cancer; other markers were selected as described below for the individual markers.

### Description of the Methodology Undertaken to Uncover the Biomolecular Sequences of the Present Invention

Human ESTs and cDNAs were obtained from GenBank versions 136 (Jun. 15, 2003 ftp.ncbinih.gov/genbank/release.notes/gb136.release.notes); NCBI genome assembly of April 2003; RefSeq sequences from June 2003; Genbank version 139 (December 2003); Human Genome from NCBI (Build 34) (from October 2003); and RefSeq sequences from December 2003. With regard to GenBank sequences, the human EST sequences from the EST (GBEST) section and the human mRNA sequences from the primate (GBPRI) section were used; also the human nucleotide RefSeq mRNA sequences were used (see for example www.ncbi.nlm.nih.gov/Genbank/GenbankOverview.html and for a reference to the EST section, see www.ncbi.nlm.nih.gov/dbEST/; a general reference to dbEST, the EST database in GenBank, may be found in Boguski et al, Nat Genet. 1993 August; 4(4):332-3; all of which are hereby incorporated by reference as if fully set forth herein).

Novel splice variants were predicted using the LEADS clustering and assembly system as described in Sorek, R., Ast, G. & Graur, D. Alu-containing exons are alternatively spliced. Genome Res 12, 1060-7 (2002); U.S. Pat. No: 6,625,545; and U.S. patent application Ser. No. 10/426,002, published as US20040101876 on May 27, 2004; all of which are hereby incorporated by reference as if fully set forth herein. Briefly, the software cleans the expressed sequences from repeats, vectors and immunoglobulins. It then aligns the expressed sequences to the genome taking alternatively splicing into account and clusters overlapping expressed sequences into “clusters” ₜhat represent genes or partial genes.

These were annotated using the GeneCarta (Compugen, Tel-Aviv, Israel) platform. The GeneCarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

A brief explanation is provided with regard to the method of selecting the candidates. However, it should be noted that this explanation is provided for descriptive purposes only, and is not intended to be limiting in any way. The potential markers were identified by a computational process that was designed to find genes and/or their splice variants that are differentially expressed in cancer tissues as opposed to non-cancerous. Various parameters related to the information in the EST libraries, determined according to classification by library annotation, were used to assist in locating genes and/or splice variants thereof that are specifically and/or differentially expressed in heart tissues. The detailed description of the selection method and of these parameters is presented in Example 1 below.

Selecting Candidates with Regard to Cancer

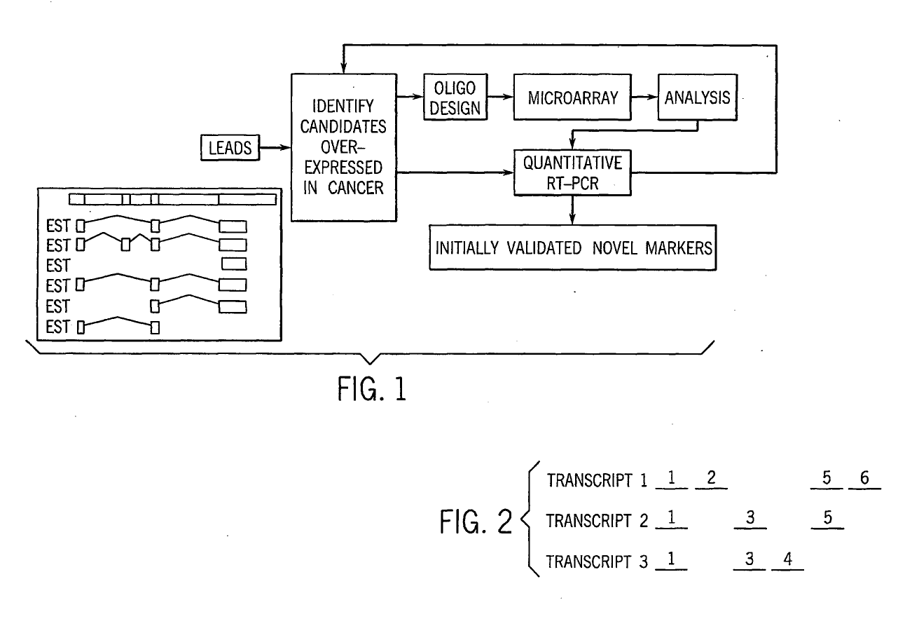

A brief explanation is provided with regard to a non-limiting method of selecting the candidates for cancer diagnostics. However, it should noted that this explanation is provided for descriptive purposes only, and is not intended to be limiting in any way. The potential markers were identified by a computational process that was designed to find genes and/or their splice variants that are over-expressed in tumor tissues, by using databases of expressed sequences. Various parameters related to the information in the EST libraries, determined according to a manual classification process, were used to assist in locating genes and/or splice variants thereof that are over-expressed in cancerous tissues. The detailed description of the selection method is presented in Example 1 below. The cancer biomarkers selection engine and the following wet validation stages are schematically summarized in FIG. 1.

### Example 1

### Identification of Differentially Expressed Gene Products—Algorithm

In order to distinguish between differentially expressed gene products and constitutively expressed genes (i.e., house keeping genes) an algorithm based on an analysis of frequencies was configured. A specific algorithm for identification of transcripts over expressed in cancer is described hereinbelow.

Dry Analysis

Library annotation—EST libraries are manually classified according to:
(i) Tissue origin
(ii) Biological source—Examples of frequently used biological sources for construction of EST libraries include cancer cell-lines; normal tissues; cancer tissues; fetal tissues; and others such as normal cell lines and pools of normal cell-lines, cancer cell-lines and combinations thereof. A specific description of abbreviations used below with regard to these tissues/cell lines etc. is given above.
(iii) Protocol of library construction—various methods are known in the art for library construction including normalized library construction; non-normalized library construction; subtracted libraries; ORESTES and others. It will be appreciated that at times the protocol of library construction is not indicated.

The following rules are followed:

EST libraries originating from identical biological samples are considered as a single library.

EST libraries which include above-average levels of DNA contamination are eliminated.

Dry computation—development of engines which are capable of identifying genes and splice variants that are temporally and spacially expressed.

Clusters (genes) having at least five sequences including at least two sequences from the tissue of interest are analyzed.

### Example 2

### Identification of Genes Over Expressed in Cancer.

Two different scoring algorithms were developed.

Libraries score—candidate sequences which are supported by a number of cancer libraries, are more likely to serve as specific and effective diagnostic markers.

The basic algorithm—for each cluster the number of cancer and normal libraries contributing sequences to the cluster was counted. Fisher exact test was used to check if cancer libraries are significantly over-represented in the cluster as compared to the total number of cancer and normal libraries.

Library counting: Small libraries (e.g., less than 1000 sequences) were excluded from consideration unless they participate in the cluster. For this reason, the total number of libraries is actually adjusted for each cluster.

Clones no. score—Generally, when the number of ESTs is much higher in the cancer libraries relative to the normal libraries it might indicate actual over-expression.

The algorithm—

Clone counting: For counting EST clones each library protocol class was given a weight based on our belief of how much the protocol reflects actual expression levels:

(i) non-normalized: 1

(ii) normalized: 0.2

(iii) all other classes: 0.1

Clones number score—The total weighted number of EST clones from cancer libraries was compared to the EST clones from normal libraries. To avoid cases where one library contributes to the majority of the score, the contribution of the library that gives most clones for a given cluster was limited to 2 clones.

The score was computed as

$$\frac{c + 1}{C} / \frac{n + 1}{N}$$

where:

c—weighted number of “cancer” clones in the cluster.

C—weighted number of clones in all “cancer” libraries.

n—weighted number of “normal” clones in the cluster.

N—weighted number of clones in all “normal” libraries.

Clones number score significance—Fisher exact test was used to check if EST clones from cancer libraries are significantly over-represented in the cluster as compared to the total number of EST clones from cancer and normal libraries.

Two search approaches were used to find either general cancer-specific candidates or tumor specific candidates.
Libraries/sequences originating from tumor tissues are counted as well as libraries originating from cancer cell-lines (“normal” cell-lines were ignored).
Only libraries/sequences originating from tumor tissues are counted

### Example 3

### Identification of Tissue Specific Genes

For detection of tissue specific clusters, tissue libraries/sequences were compared to the total number of libraries/sequences in cluster. Similar statistical tools to those described in above were employed to identify tissue specific genes. Tissue abbreviations are the same as for cancerous tissues, but are indicated with the header “normal tissue”.

The algorithm—for each tested tissue T and for each tested cluster the following were examined:

1. Each cluster includes at least 2 libraries from the tissue T. At least 3 clones (weighed—as described above) from tissue T in the cluster; and

2. Clones from the tissue T are at least 40% from all the clones participating in the tested cluster

Fisher exact test P-values were computed both for library and weighted clone counts to check that the counts are statistically significant.

### Example 4

### Oligonucleotide-Based Micro-Array Experiment Protocol

### Microarray Fabrication

Microarrays (chips) were printed by pin deposition using the MicroGrid II MGII 600 robot from BioRobotics Limited (Cambridge, UK). 50-mer oligonucleotides target sequences were designed by Compugen Ltd. (Tel-Aviv, as described by A. Shoshan et al, “Optical technologies and informatics”, Proceedings of SPIE. Vol 4266, pp. 86-95 (2001). The designed oligonucleotides were synthesized and purified by desalting with the Sigma-Genosys system (The Woodlands, Tex., US) and all of the oligonucleotides were joined to a C6 amino-modified linker at the 5′ end, or being attached directly to CodeLink slides (Cat #25-6700-01. Amersham Bioscience, Piscataway, N.J., US). The 50-mer oligonucleotides, forming the target sequences, were first suspended in Ultra-pure DDW (Cat #01-866-1A Kibbutz Beit-Haemek, Israel) to a concentration of 50 μM. Before printing the slides, the oligonucleotides were resuspended in 300 mM sodium phosphate (pH 8.5) to final concentration of 150 mM and printed at 35-40% relative humidity at 21° C.

Each slide contained a total of 9792 features in 32 subarrays. Of these features, 4224 features were sequences of interest according to the present invention and negative controls that were printed in duplicate. An additional 288 features (96 target sequences printed in triplicate) contained housekeeping genes from Human Evaluation Library2, Compugen Ltd., Israel. Another 384 features are E. coli spikes 1-6, which are oligos to E-Coli genes which are commercially available in the Array Control product (Array control-sense oligo spots, Ambion Inc. Austin, Tex. Cat #1781, Lot #112K06).

### Post-Coupling Processing of Printed Slides

After the spotting of the oligonucleotides to the glass (CodeLink) slides, the slides were incubated for 24 hours in a sealed saturated NaCl humidification chamber (relative humidity 70-75%).

Slides were treated for blocking of the residual reactive groups by incubating them in blocking solution at 50° C. for 15 minutes (10 ml/slide of buffer containing 0.1M Tris, 50 mM ethanolamine, 0.1% SDS). The slides were then rinsed twice with Ultra-pure DDW (double distilled water). The slides were then washed with wash solution (10 ml/slide. 4×SSC, 0.1% SDS)) at 50° C. for 30 minutes on the shaker. The slides were then rinsed twice with Ultra-pure DDW, followed by drying by centrifugation for 3 minutes at 800 rpm.

Next, in order to assist in automatic operation of the hybridization protocol, the slides were treated with Ventana Discovery hybridization station barcode adhesives. The printed slides were loaded on a Bio-Optica (Milan, Italy) hematology staining device and were incubated for 10 minutes in 50 ml of 3-Aminopropyl Triethoxysilane (Sigma A3648 lot #122K589). Excess fluid was dried and slides were then incubated for three hours in 20 mm/Hg in a dark vacuum desiccator (Petco 2251, Ted Pella, Inc. Redding Calif.).

The following protocol was then followed with the Genisphere 900-RP (random primer), with mini elute columns on the Ventana Discovery HybStation™, to perform the microarray experiments. Briefly, the protocol was performed as described with regard to the instructions and information provided with the device itself. The protocol included cDNA synthesis and labeling. cDNA concentration was measured with the TBS-380 (Turner Biosystems. Sunnyvale, Calif.) PicoFlour, which is used with the OliGreen ssDNA Quantitation reagent and kit.

Hybridization was performed with the Ventana Hybridization device, according to the provided protocols (Discovery Hybridization Station Tuscon Ariz.).

The slides were then scanned with GenePix 4000B dual laser scanner from Axon Instruments Inc., and analyzed by GenePix Pro 5.0 software.

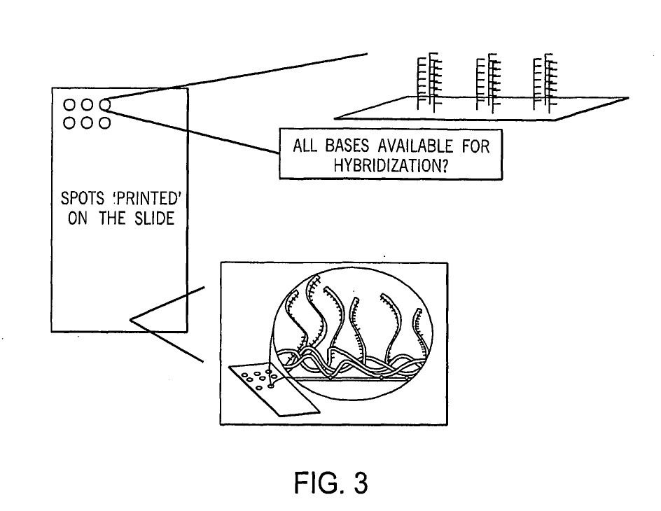

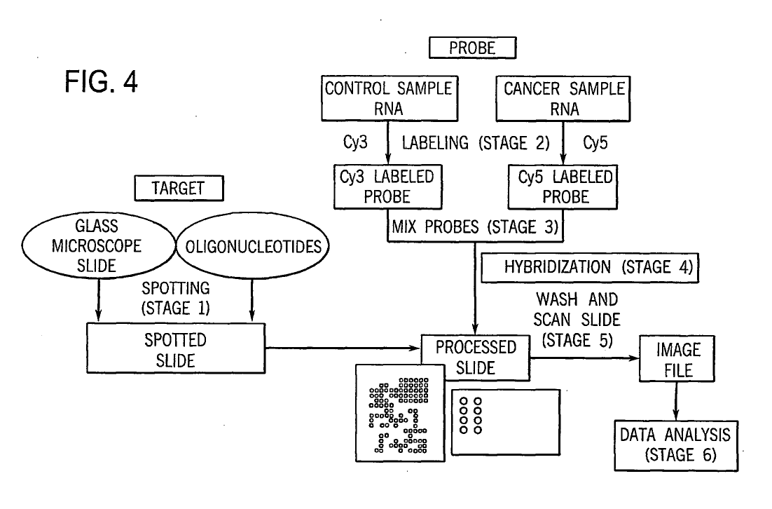

Schematic summary of the oligonucleotide based microarray fabrication and the experimental flow is presented in FIGS. 3 and 4.

Briefly, as shown in FIG. 3, DNA oligonucleotides at 25 uM were deposited (printed) onto Amersham ‘CodeLink’ glass slides generating a well defined ‘spot’. These slides are covered with a long-chain, hydrophilic polymer chemistry that creates an active 3-D surface that covalently binds the DNA oligonucleotides 5′-end via the C6-amine modification. This binding ensures that the full length of the DNA oligonucleotides is available for hybridization to the cDNA and also allows lower background, high sensitivity and reproducibility.

FIG. 4 shows a schematic method for performing the microarray experiments. It should be noted that stages on the left-hand or right-hand side may optionally be performed in any order, including in parallel, until stage 4 (hybridization). Briefly, on the left-hand side, the target oligonucleotides are being spotted on a glass microscope slide (although optionally other materials could be used) to form a spotted slide (stage 1). On the right hand side, control sample RNA and cancer sample RNA are Cy3 and Cy5 labeled, respectively (stage 2), to form labeled probes. It should be noted that the control and cancer samples come from corresponding tissues (for example, normal prostate tissue and cancerous prostate tissue). Furthermore, the tissue from which the RNA was taken is indicated below in the specific examples of data for particular clusters, with regard to overexpression of an oligonucleotide from a “chip” (microarray), as for example “prostate” for chips in which prostate cancerous tissue and normal tissue were tested as described above. In stage 3, the probes are mixed. In stage 4, hybridization is performed to form a processed slidₑ. In stage 5, the slide is washed and scanned to form an image file, followed by data analysis in stage 6.

### Example 5

Diseases and Conditions that may be Diagnosed with One or More Variants According to the Present Invention

### Cardiovascular and Cerebrovascular Conditions

Various examples are listed below for conditions that affect the vascular system, including various cardiovascular and cerebrovascular conditions, for which one or more variants according to the present invention may have a diagnostic utility. Based on these diseases mechanisms and the correlation between the known proteins and the cardiovascular and cerebrovascular conditions, such correlation was predicted also for one or more variants according to the present invention, as described below. Each variant marker of the present invention described herein as potential marker for cardiovascular conditions, might optionally be used alone or in combination with one or more other variant markers described herein, and or in combination with known markers for cardiovascular conditions, including but not limited to Heart-type fatty acid binding protein (H-FABP), Angiotensin, C-reactive protein (CRP), myeloperoxidase (MPO), and/or in combination with the known protein(s) for the variant marker as described herein. Each variant marker of the present invention described herein as potential marker for cerebrovascular conditions, might optionally be used alone or in combination with one or more other variant markers described herein, and or in combination with known markers for cerebrovascular conditions, including but not limited to CRP, S100b, BNGF, CD40, MCP1, N-Acetyl-Aspartate (NAA), N-methyl-d-aspartate (NMDA) receptor antibodies (NR2Ab), and/or in combination with the known protein(s) for the variant marker as described herein.

### Myocardial Infarction

CO3950 variants, R15601 variants and/or T11811 variants are potential markers for myocardial infarction. Other conditions that may be diagnosed by these markers or variants of them include but are not limited to the presence, risk and/or extent of the following:
1. Myocarditis—in myocarditis cardiac muscle cells can go through cell lysis and leakage with the release of intracellular content to the extracellular space and blood, a similar process as happens in myocardial infarction (see also extended description below).
2. Angina—stable or unstable, as the reduction of oxygen delivery to part of the heart often leads to local ischemic conditions that facilitate leakage of intracellular content.
3. Traumatic injury to myocardial tissue—blunt or penetrating, may also result in myocardial cell leakage.
4. Opening an occluded coronary artery following thrombolytic therapy—If such treatment is successful, proteins and other products of the local tissue are washed into the blood and can be detected there.
5. Cardiomyopathy—which is characterized by slow degeneration of the heart muscle (see also extended description below).
6. Myocardial injury after rejection of heart transplant.
7. Congestive heart failure where heart myocytes slowly degenerate (as had been shown for Troponin-I; see also extended description below).
8. Future cardiovascular disease (as a risk factor).
9. Conditions which have similar clinical symptoms as myocardial infarction and where the differential diagnosis between them and myocardial infarction is of clinical importance including but not limited to:
   a. Clinical symptoms resulting from lung related tissue (e.g. Pleuritis, pulmonary embolism)
   b. Musculoskeletal origin of pain
   c. Clinical symptoms resulting from heart related tissue which are not due to myocardial infarction, e.g. acute pericarditis
   d. Upper abdominal pain from abdominal organs including but nor limited to esophagitis, gastro-esophageal reflux, gastritis, gastric ulcer, duodenitis, duodenal ulcer, enteritis, gastroenteritis, cholecystitis, cholelithiasis, cholangiolithiasis, pancreatitis, splenic infarction, splenic trauma, Aortic dissection.

One or more of these markers (variants according to the present invention) may optionally be used a tool to decide on treatment options e.g. anti platelet inhibitors (as has been shown for Troponin-I); as a tool in the assessment of pericardial effusion; and/or as a tool in the assessment of endocarditis and/or rheumatic fever, where progressive damage to the heart muscle may occur.

### Cardiomyopathy and Myocarditis

Cardiomyopathy may be treated with the polynucleotides/polypeptides and/or methods of this invention. Cardiomyopathy is a general diagnostic term designating primary myocardial disease which may progress to heart failure. The disease comprises inflammatory cardiomyopathies, cardiomyopathies resulting from a metabolic disorder such as a nutritional deficiency or by altered endocrine function, exposure to toxic substances, for example from alcohol or exposure to cobalt or lead, infiltration and deposition of abnormal. In some embodiments, the marker(s) for diagnosis of cardiomyopathy and myocarditis, and related conditions as described herein, may optionally be selected from the group consisting of C03950 variants, R15601 variants and/or T11811 variants

### Congestive Heart Failure (CHF)

C03950 variants, R15601 variants and/or T11811 variants are potential markers for, and may be used to treat, etc., CHF.

The invention provides a means for the identification/prognostication, etc., of a number of conditions including the assessment of the presence, risk and/or extent of the following:
1. A risk factor for sudden cardiac death, from arrhythmia or any other heart related reason.
2. Rejection of a transplanted heart.
3. Conditions that lead to heart failure including but not limited to myocardial infarction, angina, arrhythmias, valvular diseases, atrial and/or ventricular septal defects.
4. Conditions that cause atrial and or ventricular wall volume overload. Wall stretch results in enhanced secretion of cardiac extracellular regulators. Such conditions include but are not limited to systemic arterial hypertension, pulmonary hypertension and pulmonary embolism.
5. Conditions which have similar clinical symptoms as heart failure and as states that cause atrial and or ventricular pressure-overload, where the differential diagnosis between these conditions to the latter is of clinical importance including but not limited to breathing difficulty and/or hypoxia due to pulmonary disease, anemia or anxiety.

### Cancerous Conditions

Various non-limiting examples are given below of cancerous conditions for which one or more variants according to the present invention may have a diagnostic, or therapeutic utility.

### Ovarian Cancer

Ovarian cancer causes more deaths than any other cancer of the female reproductive system, however, only 25% of ovarian cancers are detected in stage I. No single marker has been shown to be sufficiently sensitive or specific to contribute to the diagnosis of ovarian cancer.

In one embodiment, the markers of this invention are utilized alone, or in combination with other markers, for the diagnosis, treatment or assessment of prognosis of ovarian cancer. Such other markers may comprise CA-125 or mucin 16, CA-50, CA 54-61, CA-195 and CA 19-9, STN and TAG-72, kallikreins, cathepsin L, urine gonadotropin, inhibins, cytokeratins, such as TPA and TPS, members of the Transforming Growth Factors (TGF) beta superfamily, Epidermal Growth Factor, p53 and HER-2 or any combination thereof.

Immunohistochemistry may be used to assess the origin of the tumor and staging as part of the methods of this invention, and as protected uses for the polypeptides of this invention.

In some embodiments, this invention provides polypeptides/polynucleotides which serves as markers for ovarian cancer. In some embodiments, the marker is any polypeptide/polynucleotide as described herein. In some embodiments, the marker is D12115, or variants as described herein or markers related thereto. Each variant marker of the present invention described herein may be used alone or in combination with one or more other variant ovarian cancer described herein, and/or in combination with known markers for ovarian cancer, as described herein. Diagnosis of ovarian cancer and/or of other conditions that may be diagnosed by these markers or variants of them, include but are not limited to the presence, risk and/or extent of the following:
1. The identification of a metastasis of unknown origin which originated from a primary ovarian cancer.
2. As a marker to distinguish between different types of ovarian cancer, therefore potentially affect treatment choice (e.g. discrimination between epithelial tumors and germ cell tumors).
3. As a tool in the assessment of abdominal mass and in particular in the differential diagnosis between a benign and malignant ovarian cysts.
4. As a tool for the assessment of infertility.
5. Other conditions that may elevate serum levels of ovary related markers. These include but are not limited to: cancers of the endometrium, cervix, fallopian tubes, pancreas, breast, lung and colon; nonmalignant conditions such as pregnancy, endometriosis, pelvic inflammatory disease and uterine fibroids.
6. Conditions which have similar symptoms, signs and complications as ovarian cancer and where the differential diagnosis between them and ovarian cancer is of clinical importance including but not limited to:
   a. Non-malignant causes of pelvic mass. Including, but not limited to: benign (functional) ovarian cyst, uterine fibroids, endometriosis, benign ovarian neoplasms and inflammatory bowel lesions
   b. Any condition suggestive of a malignant tumor including but not limited to anorexia, cachexia, weight loss, fever, hypercalcemia, skeletal or abdominal pain, paraneoplastic syndrome.
   c. Ascites.
7. Prediction of patient's drug response
8. As surrogate markers for clinical outcome of a treated cancer.
9. Screening for early detection of ovarian cancer.

### Breast Cancer

Breast cancer is the most commonly occurring cancer in women, comprising almost a third of all malignancies in females. In one embodiment, the polypeptides and/or polynucleotides of this invention are utilized alone, or in combination with other markers, for the diagnosis, treatment or assessment of prognosis of breast cancer. In one embodiment, the polypeptides and/or polynucleotides serve as markers of disease.

Such markers may be used alone, or in combination with other known markers for breast cancer, including, inter alis, Mucin1 (measured as CA 15-3), CEA (CarcinoEmbryonic Antigen), HER-2, CA125, CA 19-9, PCNA, Ki-67, E-Cadherin, Cathepsin D, TFF1, epidermal growth factor receptor (EGFR), cyclin E, p53, bcl-2, vascular endothelial growth factor, urokinase-type plasminogen activator-1, survivin, or any combination thereof, and includes use of any compound which detects or quantifies the same. ESR (Erythrocyte Sedimentation Rate) values may be obtained, and comprise the marker panel for breast cancer.

In some embodiments, the polypeptides/polynucleotides of this invention serve as prognosticators, in identifying, inter alia, patients at minimal risk of relapse, patients with a worse prognosis, or patients likely to benefit from specific treatments.

There are some non-cancerous pathological conditions which represent an increased risk factor for development breast cancer, and as such, patients with these conditions may be evaluated using the polypeptides/polynucleotides and according to the methods of this invention, for example, as part of the screening methods of this invention, Some of these conditions include, but are not limited to ductal hyperplasia without atypia, atypical hyperplasia, and others.

In some embodiments, the polypeptides/polynucleotides of this invention serve as markers for breast cancer, including, but not limited to: D12115 or homologues thereof. In some embodiments, the D12115 or polynucleotides encoding the same, can be used alone or in combination with any other desired marker, including, inter alia, Calcitonin, CA15-3 (Mucin1), CA27-29, TPA, a combination of CA 15-3 and CEA, CA 27.29 (monoclonal antibody directed against MUC1), Estrogen 2 (beta), HER-2 (c-erbB2), or any combinations thereof.

In some embodiments, the polypeptides/polynucleotides of this invention may be useful in, inter alia, assessing the presence, risk and/or extent of the following:
1. The identification of a metastasis of unknown origin which originated from a primary breast cancer tumor.
2. In the assessment of lymphadenopathy, and in particular axillary lymphadenopathy.
3. As a marker to distinguish between different types of breast cancer, therefore potentially affect treatment choice (e.g. as HER-2)
4. As a tool in the assessment of palpable breast mass and in particular in the differential diagnosis between a benign and malignant breast mass.
5. As a tool in the assessment of conditions affecting breast skin (e.g. Paget's disease) and their differentiation from breast cancer.
6. As a tool in the assessment of breast pain or discomfort resulting from either breast cancer or other possible conditions (e.g. Mastitis, Mondors syndrome).
7. Other conditions not mentioned above which have similar symptoms, signs and complications as breast cancer and where the differential diagnosis between them and breast cancer is of clinical importance including but not limited to:
   a. Abnormal mammogram and/or nipple retraction and/or nipple discharge due to causes other than breast cancer. Such causes include but are not limited to benign breast masses, melanoma, trauma and technical and/or anatomical variations.
   b. Any condition suggestive of a malignant tumor including but not limited to anorexia, cachexia, weight loss, fever, hypercalcemia, paraneoplastic syndrome.

Lymphadenopathy, weight loss and other signs and symptoms associated with breast cancer but originate from diseases different from breast cancer including but not limited to other malignancies, infections and autoimmune diseases.
8. Prediction of patient's drug response
9. As surrogate markers for clinical outcome of a treated cancer.
10. Screening for early detection of breast cancer.

### Lung Cancer

Lung cancer is the primary cause of cancer death among both men and women in the U. S. In one embodiment, the polypeptides and/or polynucleotides of this invention are utilized alone, or in combination with other markers, for the diagnosis, treatment or assessment of prognosis of lung cancer. In one embodiment, the term “lung cancer” is to be understood as encompassing small cell or non-small cell lung cancers, including adenocarcinomas, bronchoalveolar-alveolar, squamous cell and large cell carcinomas.

In some embodiments, the polypeptides/polynucleotides of this invention are utilized in conjunction with other screening procedures, as well as the use of other markers, for the diagnosis, or assessment of prognosis of lung cancer in a subject. In some embodiments, such screening procedures may comprise the use of chest x-rays, analysis of the type of cells-contained in sputum, fiberoptic examination of the bronchial passages, or any combination thereof. Such evaluation in turn may impact the type of treatment regimen pursued, which in turn may reflect the type and stage of the cancer, and include surgery, radiation therapy and/or chemotherapy.

Current radiotherapeutic agents, chemotherapeutic agents and biological toxins are potent cytotoxins, yet do not discriminate between normal and malignant cells, producing adverse effects and dose-limiting toxicities. In some embodiments of this invention, the polypeptides/polynucleotides provide a means for more specific targeting to neoplastic versus normal cells.

In some embodiments, the polypeptides for use in the diagnosis, treatment and/or assessment of progression of lung cancer may comprise: D12115, N43992 or homologous thereof, or polynucleotides encoding the same. In some embodiments, these polypeptides/polynucleotides may be used alone or in combination with one or more other appropriate markers, including, inter alia, other polypeptides/polynucleotides of this invention. In some embodiments, such use may be in combination with other known markers for lung cancer, including but not limited to CEA, CA15-3, Beta-2-microglobulin, CA19-9, TPA, and/or in combination with native sequences associated with the polypeptides/polynucleotides of this invention, as herein described.

In some embodiments, the polypeptides/polynucleotides of this invention may be useful in, inter alia, assessing the presence, risk and/or extent of the following:
1. The identification of a metastasis of unknown origin which originated from a primary lung cancer.
2. The assessment of a malignant tissue residing in the lung that is from a non-lung origin, including but not limited to: osteogenic and soft tissue sarcomas; colorectal, uterine, cervix and corpus tumors; head and neck, breast, testis and salivary gland cancers; melanoma; and bladder and kidney tumors.
3. Distinguishing between different types of lung cancer, therefore potentially affect treatment choice (e.g. small cell vs. non small cell tumors).
4. Unexplained dyspnea and/or chronic cough and/or hemoptysis, and analysis thereof.
5. Differential diagnosis of the origin of a pleural effusion.
6. Conditions which have similar symptoms, signs and complications as lung cancer and where the differential diagnosis between them and lung cancer is of clinical importance including but not limited to:
   a. Non-malignant causes of lung symptoms and signs. Symptoms and signs include, but are not limited to: lung lesions and infiltrates, wheeze, stridor.
   b. Other symptoms, signs and complications suggestive of lung cancer, such as tracheal obstruction, esophageal compression, dysphagia, recurrent laryngeal nerve paralysis, hoarseness, phrenic nerve paralysis with elevation of the hemidiaphragm and Homer syndrome.
   c. Any condition suggestive of a malignant tumor including but not limited to anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.
7. Prediction of patient's drug response
8. As surrogate markers for clinical outcome of a treated cancer.
9. Screening for early detection of lung cancer.

### Candidate Marker Examples Section

This section relates to examples of sequences according to the present invention, including illustrative methods of selection thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The markers of the present invention were tested with regard to their expression in various cancerous and non-cancerous tissue samples. Unless otherwise noted, all experimental data relates to variants of the present invention, named according to the segment being tested (as expression was tested through RT-PCR as described). A description of the samples used in the ovarian cancer testing panel is provided in Table 1_{—}1 below. A description of the samples used in the lung cancer testing panel is provided in Table 1_{—}2 or Table 1_{—}5 below. A description of the samples used in the breast cancer testing panel is provided in Table 1_{—}3 below. A description of the samples used in the colon cancer testing panel is provided in Table 1_{—}4 below. The key for the table 1_{—}5is listed in tables 1_{—}5_{—}1below. A description of the samples used in the normal tissue panel is provided in Table 1_{—}6 and 1_{—}7 below. A description of samples used for microarray analysis is provided in Table 1_{—}8 for breast panel and 1_{—}9 for ovary panel. Tests were then performed as described in the “Materials and Experimental Procedures” section below.

TABLE 1_1
Tissue samples in ovarian cancer testing panel

| Sample name | Lot number | Source | Pathology | Grade | age |
|---|---|---|---|---|---|
| 33-B-Pap Sero CystAde | A503175 | BioChain | Serous papillary cystadenocarcinoma | 1 | 41 |
| G1 | | | | | |
| 41-G-Mix Sero/Muc/Endo | 98-03-G803 | GOG | Mixed epithelial cystadenocarcinoma with mucinous, | 2 | 38 |
| G2 | | | endometrioid, squamous and papillary serous (Stage2) | | |
| 35-G-Endo Adeno G2 | 94-08-7604 | GOG | Endometrioid adenocarcinoma | 2 | 39 |
| 14-B-Adeno G2 | A501111 | BioChain | Adenocarcinoma | 2 | 41 |
| 12-B-Adeno G3 | A406023 | Biochain | Adenocarcinoma | 3 | 45 |
| 40-G-Mix Sero/Endo G2 | 95-11-G006 | GOG | Papillary serous and endometrioid cystadenocarcinoma | 2 | 49 |
| | | | (Stage3C) | | |
| 4-A-Pap CystAdeno G2 | ILS-7286 | ABS | Papillary cystadenocarcinoma | 2 | 50 |
| 3-A-Pap Adeno G2 | ILS-1431 | ABS | Papillary adenocarcinoma | 2 | 52 |
| 2-A-Pap Adeno G2 | ILS-1408 | ABS | Papillary adenocarcinoma | 2 | 53 |
| 5-G-Adeno G3 | 99-12-G432 | GOG | Adenocarcinoma (Stage3C) | 3 | 46 |
| 11-B-Adeno G3 | A407068 | Biochain | Adenocarcinoma | 3 | 49 |
| 39--G-Mix Sero/Endo G3 | 2001-12-G037 | GOG | Mixed serous and endometrioid adenocarcinoma | 3 | 49 |
| 29-G-Sero Adeno G3 | 2001-12-G035 | GOG | Serous adenocarcinoma (Stage3A) | 3 | 50 |
| 70-G-Pap Sero Adeno G3 | 95-08-G069 | GOG | Papillary serous adenocarcinoma | 3 | 50 |
| 6-A-Adeno G3 | A0106 | ABS | adenocarcinoma | 3 | 51 |
| 31-B-Pap Sero CystAde | A503176 | BioChain | Serous papillary cystadenocarcinoma | 3 | 52 |
| G3 | | | | | |
| 25-A-Pap Sero Adeno G3 | N0021 | ABS | Papillary serous adenocarcinoma (StageT3CN1MX) | 3 | 55 |
| 37-G-Mix Sero/Endo G3 | 2002-05-G513 | GOG | Mixed serous and endometrioid adenocarcinoma | 3 | 56 |
| 7-A-Adeno G3 | IND-00375 | ABS | adenocarcinoma | 3 | 59 |
| 8-B-Adeno G3 | A501113 | BioChain | adenocarcinoma | 3 | 60 |
| 10-B-Adeno G3 | A407069 | Biochain | Adenocarcinoma | 3 | 60 |
| 38-G-Mix Sero/Endo G3 | 2002-05-G509 | GOG | Mixed serous and endometrioid adenocarcinoma of mullerian | 3 | 64 |
| | | | (Stage3C) | | |
| 13-G-Adeno G3 | 94-05-7603 | GOG | Poorly differentiated adenocarcinoma from primary peritoneal | 3 | 67 |
| 24-G-Pap Sero Adeno G3 | 2001-07-G801 | GOG | Papillary serous adenocarcinoma | 3 | 68 |
| 34-G-Pap Endo Adeno G3 | 95-04-2002 | GOG | Papillary endometrioid adenocarcinoma (Stage3C) | 3 | 68 |
| 30-G-Pap Sero Adeno G3 | 2001-08-G011 | GOG | Papillary serous carcinoma (Stage1C) | 3 | 72 |
| 1-A-Pap Adeno G3 | ILS-1406 | ABS | Papillary adenocarcinoma | 3 | 73 |
| 9-G-Adeno G3 | 99-06-G901 | GOG | Adenocarcinoma (maybe serous) | 3 | 84 |
| 32-G-Pap Sero CystAde | 93-09-4901 | GOG | Serous papillary cystadenocarcinoma | 3 | 67 |
| G3 | | | | | |
| 66-G-Pap Sero Adeno G3 | 2000-01-G413 | GOG | Papillary serous carcinoma (metastasis of primary peritoneum) | 3 | 67 |
| SIV | | | (Stage4) | | |
| 19-B-Muc Adeno G3 | A504085 | BioChain | Mucinous adenocarcinoma | 3 | 34 |
| 21-G-Muc CystAde G2-3 | 95-10-G020 | GOG | Mucinous cystadenocarcinoma (Stage2) | 2-3 | 44 |
| 18-B-Muc Adeno G3 | A504083 | BioChain | Mucinous adenocarcinoma | 3 | 45 |
| 20-A-Pap Muc CystAde | USA-00273 | ABS | Papillary mucinous cystadenocarcinoma | | 46 |
| 17-B-Muc Adeno G3 | A504084 | BioChain | Mucinous adenocarcinoma | 3 | 51 |
| 22-A-Muc CystAde G2 | A0139 | ABS | Mucinous cystadenocarcinoma (Stage1C) | 2 | 72 |
| 43-G-Clear cell Adeno G3 | 2001-10-G002 | GOG | Clear cell adenocarcinoma | 3 | 74 |
| 44-G-Clear cell Adeno | 2001-07-G084 | GOG | Clear cell adenocarcinoma (Stage3A) | | 73 |
| 15-B-Adeno G3 | A407065 | BioChain | Carcinoma | 3 | 27 |
| 16-Ct-Adeno | 1090387 | Clontech | Carcinoma NOS | NA | 58 |
| 23-A-Muc CystAde G3 | VNM-00187 | ABS | Mucinous cystadenocarcinoma with low malignant | 3 | 45 |
| 42-G-Adeno borderline | 98-08-G001 | GOG | Epithelial adenocarcinoma of borderline malignancy | | 46 |
| 63-G-Sero | 2000-10-G620 | GOG | Serous CysAdenoFibroma of borderline malignancy | | 71 |
| CysAdenoFibroma | | | | | |
| 62-G-Ben Muc | 99-10-G442 | GOG | Benbin mucinus cysadenoma | | 32 |
| CysAdenoma | | | | | |
| 60-G-Muc CysAdenoma | 99-01-G043 | GOG | Mucinous Cysadenoma | | 40 |
| 56-G-Ben Muc CysAdeno | 99-01-G407 | GOG | Bengin mucinus cysadenoma | | 46 |
| 64-G-Ben Sero | 99-06-G039 | GOG | Bengin Serous CysAdenoma | | 57 |
| CysAdenoma | | | | | |
| 61-G-Muc CysAdenoma | 99-07-G011 | GOG | Mucinous Cysadenoma | | 63 |
| 59-G-Sero | 98-12-G401 | GOG | Serous CysAdenoFibroma | | 77 |
| CysAdenoFibroma | | | | | |
| 51-G-N M41 | 98-03-G803N | GOG | Normal (matched tumor 98-03-G803) | | 38 |
| 75-G-N M60 | 99-01-G043N | GOG | Normal (matched tumor 99-01-G043) | | 40 |
| 49-B-N M14 | A501112 | BioChain | Normal (matched tumor A501111) | | 41 |
| 52-G-N M42 | 98-08-G001N | GOG | Normal (matched tumor 98-08-G001) | | 46 |
| 68-G-N M56 | 99-01-G407N | GOG | Normal (matched bengin 99-01-G407) | | 46 |
| 50-B-N M8 | A501114 | BioChain | Normal (matched tumor A501113) | | 60 |
| 67-G-N M38 | 2002-05-509N | GOG | Normal (matched tumor 2002-05-G509) | | 64 |
| 69-G-N M24 | 2001-07-G801N | GOG | Normal (matched tumor 2001-07-G801) | | 68 |
| 73-G-N M59 | 98-12-G401N | GOG | Normal (matched tumor 98-12-G401) | | 77 |
| 72-G-N M66 | 2000-01-G413N | GOG | Normal (matched tumor 2000-01-G413) | | |
| 45-B-N | A503274 | BioChain | Normal PM | | 41 |
| 46-B-N | A504086 | BioChain | Normal PM | | 41 |
| 71-CG-N | CG-188-7 | Ichilov | Normal PM | | 49 |
| 48-B-N | A504087 | BioChain | Normal PM | | 51 |

TABLE 1_2
Tissue samples in lung cancer testing panel

| sample name | Lot No. | source | pathology | Grade | gender/age |
|---|---|---|---|---|---|
| 1-B-Adeno G1 | A504117 | Biochain | Adenocarcinoma | 1 | F/29 |
| 2-B-Adeno G1 | A504118 | Biochain | Adenocarcinoma | 1 | M/64 |
| 95-B-Adeno G1 | A610063 | Biochain | Adenocarcinoma | 1 | F/54 |
| 12-B-Adeno G2 | A504119 | Biochain | Adenocarcinoma | 2 | F/74 |
| 75-B-Adeno G2 | A609217 | Biochain | Adenocarcinoma | 2 | M/65 |
| 77-B-Adeno G2 | A608301 | Biochain | Adenocarcinoma | 2 | M/44 |
| 13-B-Adeno G2-3 | A504116 | Biochain | Adenocarcinoma | 2-3 | M/64 |
| 89-B-Adeno G2-3 | A609077 | Biochain | Adenocarcinoma | 2-3 | M/62 |
| 76-B-Adeno G3 | A609218 | Biochain | Adenocarcinoma | 3 | M/57 |
| 94-B-Adeno G3 | A610118 | Biochain | Adenocarcinoma | 3 | M/68 |
| 3-CG-Adeno | CG-200 | Ichilov | Adenocarcinoma | | NA |
| 14-CG-Adeno | CG-111 | Ichilov | Adenocarcinoma | | M/68 |
| 15-CG-Bronch adeno | CG-244 | Ichilov | Bronchioloalveolar adenocarcinoma | | M/74 |
| 45-B-Alvelous Adeno | A501221 | Biochain | Alveolus carcinoma | | F/50 |
| 44-B-Alvelous Adeno G2 | A501123 | Biochain | Alveolus carcinoma | 2 | F/61 |
| 19-B-Squamous G1 | A408175 | Biochain | Squamous carcinoma | 1 | M/78 |
| 16-B-Squamous G2 | A409091 | Biochain | Squamous carcinoma | 2 | F/68 |
| 17-B-Squamous G2 | A503183 | Biochain | Squamous carcinoma | 2 | M/57 |
| 21-B-Squamous G2 | A503187 | Biochain | Squamous carcinoma | 2 | M/52 |
| 78-B-Squamous G2 | A607125 | Biochain | Squamous Cell Carcinoma | 2 | M/62 |
| 80-B-Squamous G2 | A609163 | Biochain | Squamous Cell Carcinoma | 2 | M/74 |
| 18-B-Squamous G2-3 | A503387 | Biochain | Squamous Cell Carcinoma | 2-3 | M/63 |
| 81-B-Squamous G3 | A609076 | Biochain | Squamous Carcinoma | 3 | m/53 |
| 79-B-Squamous G3 | A609018 | Biochain | Squamous Cell Carcinoma | 3 | M/67 |
| 20-B-Squamous | A501121 | Biochain | Squamous Carcinoma | | M/64 |
| 22-B-Squamous | A503386 | Biochain | Squamous Carcinoma | | M/48 |
| 88-B-Squamous | A609219 | Biochain | Squamous Cell Carcinoma | | M/64 |
| 100-B-Squamous | A409017 | Biochain | Squamous Carcinoma | | M/64 |
| 23-CG-Squamous | CG-109 (1) | Ichilov | Squamous Carcinoma | | M/65 |
| 24-CG-Squamous | CG-123 | Ichilov | Squamous Carcinoma | | M/76 |
| 25-CG-Squamous | CG-204 | Ichilov | Squamous Carcinoma | | M/72 |
| 87-B-Large cell G3 | A609165 | Biochain | Large Cell Carcinoma | 3 | F/47 |
| 38-B-Large cell | A504113 | Biochain | Large cell | | M/58 |
| 39-B-Large cell | A504114 | Biochain | Large cell | | F/35 |
| 82-B-Large cell | A609170 | Biochain | Large Cell Neuroendocrine | | M/68 |
| | | | Carcinoma | | |
| 30-B-Small cell carci G3 | A501389 | Biochain | small cell | 3 | M/34 |
| 31-B-Small cell carci G3 | A501390 | Biochain | small cell | 3 | F/59 |
| 32-B-Small cell carci G3 | A501391 | Biochain | small cell | 3 | M/30 |
| 33-B-Small cell carci G3 | A504115 | Biochain | small cell | 3 | M |
| 86-B-Small cell carci G3 | A608032 | Biochain | Small Cell Carcinoma | 3 | F/52 |
| 83-B-Small cell carci | A609162 | Biochain | Small Cell Carcinoma | | F/47 |
| 84-B-Small cell carci | A609167 | Biochain | Small Cell Carcinoma | | F/59 |
| 85-B-Small cell carci | A609169 | Biochain | Small Cell Carcinoma | | M/66 |
| 46-B-N M44 | A501124 | Biochain | Normal M44 | | F/61 |
| 47-B-N | A503205 | Biochain | Normal PM | | M/26 |
| 48-B-N | A503206 | Biochain | Normal PM | | M/44 |
| 49-B-N | A503384 | Biochain | Normal PM | | M/27 |
| 50-B-N | A503385 | Biochain | Normal PM | | M/28 |
| 90-B-N | A608152 | Biochain | Normal (Pool 2) PM | | pool 2 |
| 91-B-N | A607257 | Biochain | Normal (Pool 2) PM | | pool 2 |
| 92-B-N | A503204 | Biochain | Normal PM | | m/28 |
| 93-Am-N | 111P0103A | Ambion | Normal PM | | F/61 |
| 96-Am-N | 36853 | Ambion | Normal PM | | F/43 |
| 97-Am-N | 36854 | Ambion | Normal PM | | M/46 |
| 98-Am-N | 36855 | Ambion | Normal PM | | F/72 |
| 99-Am-N | 36856 | Ambion | Normal PM | | M/31 |

TABLE 1_3
Tissue samples in breast cancer testing panel

| sample name | Lot no | source | pathology | grade | age | TNM | stage |
|---|---|---|---|---|---|---|---|
| 14-A-IDC G2 | A0135T | ABS | IDC | 2 | 37 | T2N2Mx | |
| 43-B-IDC G2 | A609183 | Biochain | IDC | 2 | 40 | | |
| 54-B-IDC G2 | A605353 | Biochain | IDC | 2 | 41 | | |
| 55-B-IDC G2 | A609179 | Biochain | IDC | 2 | 42 | | |
| 47-B-IDC G2 | A609221 | Biochain | IDC | 2 | 42 | | |
| 17-A-IDC G2 | 4904020036T | ABS | IDC | 2-3 | 42 | T3N1Mx | |
| 42-A-IDC G3 | 6005020031T | ABS | IDC | 3 | 42 | T1cN0Mx | |
| 7-A-IDC G2 | 7263T | ABS | IDC | 2 | 43 | T1N0M0 | stage 1 |
| 48-B-IDC G2 | A609222 | Biochain | IDC | 2 | 44 | | |
| 53-B-IDC G2 | A605151 | Biochain | IDC | 2 | 44 | | |
| 12-A-IDC G2 | 1432T | ABS | IDC | 2 | 46 | T2N0M0 | stage 2A |
| 61-B-IDC G2 | A610029 | Biochain | IDC | 2 | 46 | | |
| 46-B-Carci G2 | A609177 | Biochain | Carcinoma | 2 | 48 | | |
| 16-A-IDC G2 | 4904020032T | ABS | IDC | 2 | 49 | T3N1Mx | |
| 62-B-IDC G2 | A609194 | Biochain | IDC | 2 | 51 | | |
| 49-B-IDC G2 | A609223 | Biochain | IDC | 2 | 54 | | |
| 32-A-Muc Carci | 7116T | ABS | Mucinous carcinoma | | 54 | T2N0M0 | stage 2A |
| 45-B-IDC G2 | A609181 | Biochain | IDC | 2 | 58 | | |
| 15-A-IDC G2 | 7259T | ABS | IDC | 2 | 59 | T3N1M0 | stage 3A |
| 52-B-ILC G1 | A605360 | Biochain | Invasive Lobular Carcinoma | 1 | 60 | | |
| 6-A-IDC G1 | 7238T | ABS | IDC | 1 | 60 | T2N0M0 | stage 2A |
| 26-A-IDC G3 | 7249T | ABS | IDC | 3 | 60 | T2N0M0 | stage 2A |
| 13-A-IDC G2 | A0133T | ABS | IDC | 2 | 63 | T2N1aMx | |
| 50-B-IDC G2 | A609224 | Biochain | IDC | 2 | 69 | | |
| 44-B-IDC G2 | A609198 | Biochain | IDC | 2 | 77 | | |
| 51-B-IDC G1 | A605361 | Biochain | IDC | 1 | 79 | | |
| 31-CG-IDC | CG-154 | Ichilov | IDC | | 83 | | |
| 27-A-IDC G3 | 4907020072T | ABS | IDC | 3 | 91 | T2N0Mx | |
| 36-A-N M7 | 7263N | ABS | Normal matched to 7T | | 43 | | |
| 40-A-N M12 | 1432N | ABS | Normal matched to 12T | | 46 | | |
| 39-A-N M15 | 7259N | ABS | Normal matched to 15T | | 59 | | |
| 35-A-N M6 | 7238N | ABS | Normal matched to 6T | | 60 | | |
| 41-A-N M26 | 7249N | ABS | Normal matched to 26T | | 60 | | |
| 57-B-N | A609233 | Biochain | Normal PM | | 34 | | |
| 59-B-N | A607155 | Biochain | Normal PM | | 35 | | |
| 60-B-N | A609234 | Biochain | Normal PM | | 36 | | |
| 63-Am-N | 26486 | Ambion | Normal PS | | 43 | | |
| 66-Am-N | 36678 | Ambion | Normal PM | | 45 | | |
| 64-Am-N | 23036 | Ambion | Normal PM | | 57 | | |
| 56-B-N | A609235 | Biochain | Normal PM | | 59 | | |
| 65-Am-N | 31410 | Ambion | Normal PM | | 63 | | |
| 67-Am-N | 073P010602086A | Ambion | Normal PM | | 64 | | |
| 58-B-N | A609232 | Biochain | Normal PM | | 65 | | |

TABLE 1_4
Tissue samples in colon cancer testing panel

| sample name | Lot No. | tissue | source | pathology | Grade | gender/age |
|---|---|---|---|---|---|---|
| 58-B-Adeno G1 | A609152 | Colon | biochain | Adenocarcinoma | 1 | M/73 |
| 59-B-Adeno G1 | A609059 | Colon | biochain | Adenocarcinoma, Ulcer | 1 | M/58 |
| 14-CG-Polypoid Adeno | CG-222 (2) | Rectum | Ichilov | Well polypoid adeocarcinoma Duke's C | | F/49 |
| G1 D-C | | | | | | |
| 17-CG-Adeno G1-2 | CG-163 | Rectum | Ichilov | Adenocarcinoma | 2 | M/73 |
| 10-CG-Adeno G1-2 D-B2 | CG-311 | Sigmod colon | Ichilov | Adenocarcinoma Astler-Coller B2. | 1-2 | M/88 |
| 11-CG-Adeno G1-2 D-C2 | CG-337 | Colon | Ichilov | Adenocarcinoma Astler-Coller C2. | 1-2 | NA |
| 6-CG-Adeno G1-2 D-C2 | CG-303 (3) | Colon | Ichilov | Adenocarcinoma Astler-Coller C2. | 1-2 | F/77 |
| 5-CG-Adeno G2 | CG-308 | Colon Sigma | Ichilov | Adenocarcinoma. | 2 | F/80 |
| 16-CG-Adeno G2 | CG-278C | colon | Ichilov | Adenocarcinoma | 2 | F/60 |
| 56-B-Adeno G2 | A609148 | Colon | biochain | Adenocarcinoma | 2 | F48 |
| 61-B-Adeno G2 | A606258 | Colon | biochain | Adenocarcinoma, Ulcer | 2 | M/41 |
| 60-B-Adeno G2 | A609058 | Colon | biochain | Adenocarcinoma, Ulcer | 2 | M/67 |
| 22-CG-Adeno G2 D-B | CG-229C | Colon | Ichilov | Adenocarcinoma Duke's B | 2 | F/55 |
| 1-CG-Adeno G2 D-B2 | CG-335 | Cecum | Ichilov | Adenocarcinoma Dukes B2. | 2 | F/66 |
| 12-CG-Adeno G2 D-B2 | CG-340 | Colon Sigma | Ichilov | Adenocarcinoma Astler-Coller B2. | 2 | M/66 |
| 28-CG-Adeno G2 D-B2 | CG-284 | sigma | Ichilov | Adenocarcinoma Duke's B2 | 2 | F/72 |
| 2-CG-Adeno G2 D-C2 | CG-307 X2 | Cecum | Ichilov | Adenocarcinoma Astler-Coller C2. | 2 | F/89 |
| 9-CG-Adeno G2 D-D | CG-297 X2 | Rectum | Ichilov | Adenocarcinoma Dukes D. | 2 | M/62 |
| 13-CG-Adeno G2 D-D | CG-290 X2 | Rectosigmoidal | Ichilov | Adenocarcinoma Dukes D. | 2 | M/47 |
| | | colon | | | | |
| 26-CG-Adeno G2 D-D | CG-283 | sigma | Ichilov | Colonic adenocarcinoma Duke's D | 2 | F/63 |
| 4-CG-Adeno G3 | CG-276 | Colon | Ichilov | Carcinoma. | 3 | M/64 |
| 53-B-Adeno G3 | A609161 | Colon | biochain | Adenocarcinoma | 3 | F/53 |
| 54-B-Adeno G3 | A609142 | Colon | biochain | Adenocarcinoma | 3 | M/53 |
| 55-B-Adeno G3 | A609144 | Colon | biochain | Adenocarcinoma | 3 | M/68 |
| 57-B-Adeno G3 | A609150 | Colon | biochain | Adenocarcinoma | 3 | F/45 |
| 72-CG-Adeno G3 | CG-309 | colon | Ichilov | Adenocarcinoma | 3 | F/88 |
| 20-CG-Adeno G3 D-B2 | CG-249 | Colon | Ichilov | Ulcerated adenocarcinoma Duke's B2 | 3 | M/36 |
| 7-CG-Adeno D-A | CG-235 | Rectum | Ichilov | Adenocarcinoma intramucosal Duke's A. | | F/66 |
| 23-CG-Adeno D-C | CG-282 | sigma | Ichilov | Mucinus adenocarcinoma Astler Coller C | | M/51 |
| 3-CG-Muc adeno D-D | CG-224 | Colon | Ichilov | Mucinois adenocarcinoma Duke's D | | M/48 |
| 18-CG-Adeno | CG-22C | Colon | Ichilov | Adenocarcinoma | | NA |
| 19-CG-Adeno | CG-19C | Colon | Ichilov | Adenocarcinoma | | NA |
| | (1) | | | | | |
| 21-CG-Adeno | CG-18C | Colon | Ichilov | Adenocarcinoma | | NA |
| 24-CG-Adeno | CG-12 (2) | Colon | Ichilov | Adenocarcinoma | | NA |
| 25-CG-Adeno | CG-2 | Colon | Ichilov | Adenocarcinoma | | NA |
| 27-CG-Adeno | CG-4 | Colon | Ichilov | Adenocarcinoma | | NA |
| 8-CG-diverticolosis, | CG-291 | Wall of sigma | Ichilov | Diverticolosis and diverticulitis of the | | F/65 |
| diverticulitis | | | | Colon | | |
| 46-CG-Crohn's disease | CG-338C | Cecum | Ichilov | Crohn's disease | | M/22 |
| 47-CG-Crohn's disease | CG-338AC | Colon | Ichilov | Crohn's disease. | | M/22 |
| 42-CG-N M20 | CG-249N | Colon | Ichilov | Normal | | M/36 |
| 43-CG-N M8 | CG-291N | Wall of sigma | Ichilov | Normal | | F/65 |
| 44-CG-N M21 | CG-18N | Colon | Ichilov | Normal | | NA |
| 45-CG-N M11 | CG-337N | Colon | Ichilov | Normal | | M/75 |
| 49-CG-N M14 | CG-222N | Rectum | Ichilov | Normal | | F/49 |
| 50-CG-N M5 | CG-308N | Sigma | Ichilov | Within normal limits | | F/80 |
| 51-CG-N M26 | CG-283N | Sigma | Ichilov | Normal | | F/63 |
| 41-B-N | A501156 | Colon | biochain | Normal PM | | M/78 |
| 52-CG-N | CG-309TR | Colon | Ichilov | Within normal limits | | F/88 |
| 62-B-N | A608273 | Colon | biochain | Normal PM | | M/66 |
| 63-B-N | A609260 | Colon | biochain | Normal PM | | M/61 |
| 64-B-N | A609261 | Colon | biochain | Normal PM | | F/68 |
| 65-B-N | A607115 | Colon | biochain | Normal PM | | M/24 |
| 66-B-N | A609262 | Colon | biochain | Normal PM | | M/58 |
| 67-B-N | A406029 | Colon | biochain | Normal PM (Pool of 10) | | |
| 69-B-N | A411078 | Colon | biochain | Normal PM (Pool of 10) | | F&M |
| 70-Cl-N | 1110101 | Colon | clontech | Normal PM (Pool of 3) | | |
| 71-Am-N | 071P10B | Colon | Ambion | Normal (IC BLEED) | | F/34 |

TABLE 1_5
Tissue samples in lung cancer testing panel

| | | | sample | | | | | | | | | | |
|---|---|---|---|---|---|---|---|---|---|---|---|---|---|
| | | | id | | | | | | | | | | |
| | | | (GCI)/ | | | | | | | | | | |
| | | | case id | TISSUE | | | | | | | | | |
| | | | (Asterand)/ | ID | RNA | | | | | | | | |
| | | | lot | (GCI)/ | ID (GCI)/ | | | | | | | | |
| | | | no. | specimen | Sample | | | | | | | | |
| | Source/ | sample | (old | ID | ID | | Diag | Specimen | | | | Tum | |
| Tissue | Delivery | name | samples) | (Asterand) | (Asterand) | Diag | remarks | location | Gr | TNM | CS | % | Gen |
| LC | GCI | 1-GC- | 7Z9V4 | 7Z9V4AYM | | Aden | BC | | | | IA | 80 | F |
| | | BAC-SIA | | | | | | | | | | | |
| LC | GCI | 2-GC- | ZW2AQ | ZW2AQARP | | Aden | BC | | | | IB | 70 | F |
| | | BAC-SIB | | | | | | | | | | | |
| LC | Bioch | 72-(44)- | A501123 | | | AC | | | 2 | UN | | | F |
| | | Bc-BAC | | | | | | | | | | | |
| LC | Bioch | 73-(45)- | A501221 | | | AC | | | UN | UN | | | F |
| | | Bc-BAC | | | | | | | | | | | |
| LC | GCI | 4-GC- | 3MOPL | 3MOPLA79 | | Aden | | | | | IA | 60 | M |
| | | Adeno- | | | | | | | | | | | |
| | | SIA | | | | | | | | | | | |
| LC | GCI | 5-GC- | KOJXD | KOJXDAV4 | | Aden | | | | | IA | 90 | F |
| | | Adeno- | | | | | | | | | | | |
| | | SIA | | | | | | | | | | | |
| LC | GCI | 6-GC- | X2Q44 | X2Q44A79 | | Aden | | | | | IA | 85 | M |
| | | Adeno- | | | | | | | | | | | |
| | | SIA | | | | | | | | | | | |
| LC | GCI | 7-GC- | 6BACZ | 6BACZAP5 | | Aden | | | | | IA | 60 | F |
| | | Adeno- | | | | | | | | | | | |
| | | SIA | | | | | | | | | | | |
| LC | GCI | 8-GC- | BS9AF | BS9AFA3E | | Aden | | | | | IA | 55 | F |
| | | Adeno- | | | | | | | | | | | |
| | | SIA | | | | | | | | | | | |
| LC | GCI | 9-GC- | UCLOA | UCLOAA9L | | Aden | | | | | IA | 80 | F |
| | | Adeno- | | | | | | | | | | | |
| | | SIA | | | | | | | | | | | |
| LC | GCI | 10-GC- | BVYK3 | BVYK3A7Z | | Aden | | | | | IA | 60 | F |
| | | Adeno- | | | | | | | | | | | |
| | | SIA | | | | | | | | | | | |
| LC | GCI | 11-GC- | U4DM4 | U4DM4AFZ | | Aden | | | | | IB | 65 | F |
| | | Adeno- | | | | | | | | | | | |
| | | SIB | | | | | | | | | | | |
| LC | GCI | 12-GC- | OWX5Y | OWX5YA3S | | Aden | | | | | IB | 90 | M |
| | | Adeno- | | | | | | | | | | | |
| | | SIB | | | | | | | | | | | |
| LC | GCI | 13-GC- | XYY96 | XYY96A6B | | Aden | | | | | IIA | 70 | F |
| | | Adeno- | | | | | | | | | | | |
| | | SIIA | | | | | | | | | | | |
| LC | GCI | 14-GC- | SO7B1 | SO7B1AIJ | | Aden | | | | | IIA | 70 | M |
| | | Adeno- | | | | | | | | | | | |
| | | SIIA | | | | | | | | | | | |
| LC | GCI | 15-GC- | QANSY | QANSYACD | | Aden | | | | | IIIA | 65 | F |
| | | Adeno- | | | | | | | | | | | |
| | | SIIIA | | | | | | | | | | | |
| LC | Bioch | 16-(95)- | A610063 | | | Aden | | | 1 | UN | | | F |
| | | BC- | | | | | | | | | | | |
| | | Adeno | | | | | | | | | | | |
| LC | Bioch | 17-(89)- | A609077 | | | Aden | | | 2-3 | UN | | | M |
| | | Bc- | | | | | | | | | | | |
| | | Adeno | | | | | | | | | | | |
| LC | Bioch | 18-(76)- | A609218 | | | Aden | | | 3 | UN | | | M |
| | | Bc- | | | | | | | | | | | |
| | | Adeno | | | | | | | | | | | |
| LC | Bioch | 74-(2)-Bc- | A504118 | | | Aden | | | 1 | UN | | | M |
| | | Adeno | | | | | | | | | | | |
| LC | Bioch | 76-(75)- | A609217 | | | Aden | | | 2 | UN | | | M |
| | | Bc- | | | | | | | | | | | |
| | | Adeno | | | | | | | | | | | |
| LC | Bioch | 77-(12)- | A504119 | | | Aden | | | 2 | UN | | | F |
| | | Bc- | | | | | | | | | | | |
| | | Adeno | | | | | | | | | | | |
| LC | Bioch | 78-(13)- | A504116 | | | Aden | | | 2-3 | UN | | | M |
| | | Bc- | | | | | | | | | | | |
| | | Adeno | | | | | | | | | | | |
| LC | Bioch | 79-(94)- | A610118 | | | Aden | | | 3 | UN | | | M |
| | | Bc- | | | | | | | | | | | |
| | | Adeno | | | | | | | | | | | |
| LC | Ichilov | 80-(3)-Ic- | CG-200 | | | Aden | | | UN | UN | | | F |
| | | Adeno | | | | | | | | | | | |
| LC | Ichilov | 81-(14)- | CG-111 | | | Aden | | | UN | UN | | | M |
| | | Ic-Adeno | | | | | | | | | | | |
| LC | Aster | 19-As-Sq- | 9220 | 9418 | 9418A1 | SCC | | | 1 | TXN0M0 | Occult | 80 | M |
| | | S0 | | | | | | | | | | | |
| LC | GCI | 20-GC- | U2QHS | U2QHSA2N | | SCC | | | | | IA | 55 | F |
| | | Sq-SIA | | | | | | | | | | | |
| LC | GCI | 21-GC- | TRQR7 | TRQR7ACD | | SCC | | | | | IB | 75 | M |
| | | Sq-SIB | | | | | | | | | | | |
| LC | Aster | 22-As-Sq- | 17581 | 32603 | 32603B1 | SCC | | | 3 | T2N0M0 | IB | 90 | M |
| | | SIB | | | | | | | | | | | |
| LC | Aster | 23-As-Sq- | 18309 | 41454 | 41454B1 | SCC | | | 2 | T2N0MX | IB | 100 | M |
| | | SIB | | | | | | | | | | | |
| LC | Aster | 24-As-Sq- | 9217 | 9415 | 9415B1 | SCC | | | 2 | T2N0M0 | IB | 90 | M |
| | | SIB | | | | | | | | | | | |
| LC | GCI | 25-GC- | RXQ1P | RXQ1PAEA | | SCC | | | | | IIB | 55 | F |
| | | Sq-SIIB | | | | | | | | | | | |
| LC | GCI | 26-GC- | KB5KH | KB5KHA6X | | SCC | | | | | IIB | 65 | M |
| | | Sq-SIIB | | | | | | | | | | | |
| LC | GCI | 27-GC- | LAYMB | LAYMBALF | | SCC | | | | | IIIA | 65 | F |
| | | Sq-SIIIA | | | | | | | | | | | |
| LC | Ichilov | 28-(23)- | CG-109 (1) | | | SCC | | | UN | UN | | | M |
| | | Ic-Sq | | | | | | | | | | | |
| LC | Ichilov | 29-(25)- | CG-204 | | | SCC | | | UN | UN | | | M |
| | | Ic-Sq | | | | | | | | | | | |
| LC | Bioch | 30-(19)- | A408175 | | | SCC | | | 1 | UN | | | M |
| | | Bc-Sq | | | | | | | | | | | |
| LC | Bioch | 31-(78)- | A607125 | | | SCC | | | 2 | UN | | | M |
| | | Bc-Sq | | | | | | | | | | | |
| LC | Bioch | 32-(16)- | A409091 | | | SCC | | | 2 | UN | | | F |
| | | Bc-Sq | | | | | | | | | | | |
| LC | Bioch | 33-(80)- | A609163 | | | SCC | | | 2 | UN | | | M |
| | | Bc-Sq | | | | | | | | | | | |
| LC | Bioch | 34-(18)- | A503387 | | | SCC | | | 2-3 | UN | | | M |
| | | Bc-Sq | | | | | | | | | | | |
| LC | Bioch | 92-(38)- | A504113 | | | LCC | | | UN | UN | | | M |
| | | Bc-LCC | | | | | | | | | | | |
| LC | Bioch | 93-(82)- | A609170 | | | LCNC | | | UN | UN | | | M |
| | | Bc-LCC | | | | | | | | | | | |
| LC | GCI | 42-GC- | QPJQL | QPJQLAF6 | | SMCC | NC | | 3 | | IB | 65 | F |
| | | SCC-SIB | | | | | | | | | | | |
| LC | Bioch | 43-(32)- | A501391 | | | SMCC | | | | UN | | | M |
| | | Bc-SCC | | | | | | | | | | | |
| LC | Bioch | 44-(30)- | A501389 | | | SMCC | | | 3 | UN | | | M |
| | | Bc-SCC | | | | | | | | | | | |
| LC | Bioch | 45-(83)- | A609162 | | | SMCC | | | UN | UN | | | F |
| | | Bc-SCC | | | | | | | | | | | |
| LC | Bioch | 46-(86)- | A608032 | | | SMCC | | | 3 | UN | | | F |
| | | Bc-SCC | | | | | | | | | | | |
| LC | Bioch | 47-(31)- | A501390 | | | SMCC | | | | UN | | | F |
| | | Bc-SCC | | | | | | | | | | | |
| LC | Bioch | 48-(84)- | A609167 | | | SMCC | | | UN | UN | | | F |
| | | Bc-SCC | | | | | | | | | | | |
| LC | Bioch | 49-(85)- | A609169 | | | SMCC | | | UN | UN | | | M |
| | | Bc-SCC | | | | | | | | | | | |
| LC | Bioch | 50-(33)- | A504115 | | | SMCC | | | | UN | | | M |
| | | Bc-SCC | | | | | | | | | | | |
| LN | Aster | 51-As-N- | 9078 | 9275 | 9275B1 | Norm-L | PS | | | | | | M |
| | | PS | | | | | | | | | | | |
| LN | Aster | 52-As-N- | 8757 | 8100 | 8100B1 | Norm-L | PM | (Right), | | | | | F |
| | | PM | | | | | | Lobe | | | | | |
| | | | | | | | | Inferior | | | | | |
| LN | Aster | 53-As-N- | 6692 | 6161 | 6161A1 | Norm-L | PM | | | | | | M |
| | | PM | | | | | | | | | | | |
| LN | Aster | 54-As-N- | 7900 | 7180 | 7180F1 | Norm-L | PM | | | | | | F |
| | | PM | | | | | | | | | | | |
| LN | Aster | 55-As-N- | 8771 | 8163 | 8163A1 | Norm-L | PM | (Left), | | | | | M |
| | | PM | | | | | | Lobe | | | | | |
| | | | | | | | | Superior | | | | | |
| LC | Bioch | 35-(81)- | A609076 | | | SCC | | | 3 | UN | | | M |
| | | Bc-Sq | | | | | | | | | | | |
| LC | Bioch | 82-(21)- | A503187 | | | SCC | | | 2 | UN | | | M |
| | | Bc-Sq | | | | | | | | | | | |
| LC | Bioch | 83-(17)- | A503183 | | | SCC | | | 2 | UN | | | M |
| | | Bc-Sq | | | | | | | | | | | |
| LC | Bioch | 84-(79)- | A609018 | | | SCC | | | 3 | UN | | | M |
| | | Bc-Sq | | | | | | | | | | | |
| LC | Bioch | 85-(22)- | A503386 | | | SCC | | | UN | UN | | | M |
| | | Bc-Sq | | | | | | | | | | | |
| LC | Bioch | 86-(20)- | A501121 | | | SCC | | | UN | UN | | | M |
| | | Bc-Sq | | | | | | | | | | | |
| LC | Bioch | 87-(88)- | A609219 | | | SCC | | | UN | UN | | | M |
| | | Bc-Sq | | | | | | | | | | | |
| LC | Bioch | 88-(100)- | A409017 | | | SCC | | | UN | UN | | | M |
| | | Bc-Sq | | | | | | | | | | | |
| LC | Ichilov | 89-(24)- | CG-123 | | | SCC | | | UN | UN | | | M |
| | | Ic-Sq | | | | | | | | | | | |
| LC | GCI | 36-GC- | AF8AL | AF8ALAAL | | LCC | | | | | IA | 85 | M |
| | | LCC-SIA | | | | | | | | | | | |
| LC | GCI | 37-GC- | O62XU | O62XUA1X | | LCC | | | | | IB | 75 | F |
| | | LCC-SIB | | | | | | | | | | | |
| LC | GCI | 38-GC- | OLOIM | OLOIMAS1 | | LCC | | | | | IB | 70 | M |
| | | LCC-SIB | | | | | | | | | | | |
| LC | GCI | 39-GC- | 1ZWSV | 1ZWSVAB9 | | LCC | | | | | IIB | 50 | M |
| | | LCC-SIIB | | | | | | | | | | | |
| LC | GCI | 40-GC- | 2YHOD | 2YHODA1H | | LCC | NSCC | | | | IIB | 95 | M |
| | | LCC-SIIB | | | | | ... | | | | | | |
| LC | GCI | 41-GC- | 38B4D | 38B4DAQK | | LCC | | | | | IIB | 90 | F |
| | | LCC-SIIB | | | | | | | | | | | |
| LC | Bioch | 90-(39)- | A504114 | | | LCC | | | UN | UN | | | F |
| | | Bc-LCC | | | | | | | | | | | |
| LC | Bioch | 91-(87)- | A609165 | | | LCC | | | 3 | UN | | | F |
| | | Bc-LCC | | | | | | | | | | | |
| LN | Aster | 56-As-N- | 13094 | 19763 | 19763A1 | Norm-L | PM | | | | | | M |
| | | PM | | | | | | | | | | | |
| LN | Aster | 57-As-N- | 19174 | 40654 | 40654A2 | Norm-L | PM | | | | | | F |
| | | PM | | | | | | | | | | | |
| LN | Aster | 58-As-N- | 13128 | 19642 | 19642A1 | Norm-L | PM | | | | | | F |
| | | PM | | | | | | | | | | | |
| LN | Aster | 59-As-N- | 14374 | 20548 | 20548C1 | Norm-L | PM | (Right), | | | | | F |
| | | PM | | | | | | Lobe | | | | | |
| | | | | | | | | Superior | | | | | |
| LN | Amb | 60-(99)- | 36856 | | | N-PM | PM | | | | | | M |
| | | Am-N | | | | | | | | | | | |
| | | PM | | | | | | | | | | | |
| LN | Amb | 61-(96)- | 36853 | | | N-PM | PM | | | | | | F |
| | | Am-N | | | | | | | | | | | |
| | | PM | | | | | | | | | | | |
| LN | Amb | 62-(97)- | 36854 | | | N-PM | PM | | | | | | M |
| | | Am-N | | | | | | | | | | | |
| | | PM | | | | | | | | | | | |
| LN | Amb | 63-(93)- | 111P0103A | | | N-PM | PM- | | | | | | F |
| | | Am-N | | | | | ICH | | | | | | |
| | | PM | | | | | | | | | | | |
| LN | Amb | 64-(98)- | 36855 | | | N-PM | PM | | | | | | F |
| | | Am-N | | | | | | | | | | | |
| | | PM | | | | | | | | | | | |
| LN | Bioch | 67-(50)- | A503385 | | | N-PM | PM | | | | | | M |
| | | Bc-N PM | | | | | | | | | | | |
| LN | Bioch | 68-(92)- | A503204 | | | N-PM | PM | | | | | | M |
| | | Bc-N PM | | | | | | | | | | | |
| LN | Bioch | 69-(91)- | A607257 | | | N-P2- | PM | | | | | | P2 |
| | | Bc-N PM | | | | PM | | | | | | | |
| LN | Bioch | 70-(90)- | A608152 | | | N-P2 | PM | | | | | | P2 |
| | | Bc-N PM | | | | PM | | | | | | | |
| LN | Bioch | 71-(48)- | A503206 | | | N-PM | PM | | | | | | M |
| | | Bc-N PM | | | | | | | | | | | |

| | | | | | | | # of | # | | | | | | | | | |
|---|---|---|---|---|---|---|---|---|---|---|---|---|---|---|---|---|---|
| | | | | | | # | Y. | Y. | | | | | | | | | |
| | | | | | Smok- | Cig. | Use | off | | | | | | | Re- | Cause | |
| Tis- | Source/ | sample | | Ethnic | ing | Per | of To- | To- | Sm | Sm | Dr | # | HT | | covery | of | Exc. |
| sue | Delivery | name | age | B | Status | day | bacco | bacco | PY? | ppl | Al | Dr | (CM) | BMI | Type | Death | Y. |
| LC | GCI | 1-GC- | 63 | WCAU | Prev | 20 | 15 | 27 | N | — | Y | 0 | 165 | 25.3 | Surg | | 2001 |
| | | BAC-SIA | | | U. | | | | | | | | | | | | |
| LC | GCI | 2-GC- | 56 | WCAU | Prev | 15 | 28 | 10 | Y | 1 | Y | 6 | 165 | 23 | Surg | | 2002 |
| | | BAC-SIB | | | U. | | | | | | | | | | | | |
| LC | Bioch | 72-(44)- | 61 | | | | | | | | | | | | | | |
| | | Bc-BAC | | | | | | | | | | | | | | | |
| LC | Bioch | 73-(45)- | 50 | | | | | | | | | | | | | | |
| | | Bc-BAC | | | | | | | | | | | | | | | |
| LC | GCI | 4-GC- | 68 | WCAU | Nev | — | — | — | N | — | N | — | 175 | 27.3 | Surg | | 2001 |
| | | Adeno- | | | U. | | | | | | | | | | | | |
| | | SIA | | | | | | | | | | | | | | | |
| LC | GCI | 5-GC- | 64 | WCAU | Prev | 15 | 40 | 7 | Y | 1 | N | 0 | 157 | 19.6 | Surg | | 2003 |
| | | Adeno- | | | U. | | | | | | | | | | | | |
| | | SIA | | | | | | | | | | | | | | | |
| LC | GCI | 6-GC- | 58 | WCAU | Prev | 10 | 47 | 0 | Y | 2 | N | — | 170 | 24.6 | Surg | | 2004 |
| | | Adeno- | | | U. | | | | | | | | | | | | |
| | | SIA | | | | | | | | | | | | | | | |
| LC | GCI | 7-GC- | 65 | WCAU | Curr | 6 | 30 | — | Y | 1 | N | — | 168 | 21 | Surg | | 2004 |
| | | Adeno- | | | U. | | | | | | | | | | | | |
| | | SIA | | | | | | | | | | | | | | | |
| LC | GCI | 8-GC- | 59 | WCAU | Curr | 20 | 40 | — | N | — | N | — | 160 | 23.9 | Surg | | 2004 |
| | | Adeno- | | | U. | | | | | | | | | | | | |
| | | SIA | | | | | | | | | | | | | | | |
| LC | GCI | 9-GC- | 69 | WCAU | Curr | 30 | 52 | — | Y | 4 | N | — | 157 | 34.8 | Surg | | 2005 |
| | | Adeno- | | | U. | | | | | | | | | | | | |
| | | SIA | | | | | | | | | | | | | | | |
| LC | GCI | 10-GC- | 60 | WCAU | Curr | 40 | 40 | — | N | — | N | — | 163 | 31.8 | Surg | | 2002 |
| | | Adeno- | | | U. | | | | | | | | | | | | |
| | | SIA | | | | | | | | | | | | | | | |
| LC | GCI | 11-GC- | 68 | WCAU | Prev | 5 | 4 | 43 | N | — | N | — | 165 | 22.3 | Surg | | 2003 |
| | | Adeno- | | | U. | | | | | | | | | | | | |
| | | SIB | | | | | | | | | | | | | | | |
| LC | GCI | 12-GC- | 69 | WCAU | Curr | 10 | — | — | | — | N | — | 183 | 30.5 | Surg | | 2002 |
| | | Adeno- | | | U. | | | | | | | | | | | | |
| | | SIB | | | | | | | | | | | | | | | |
| LC | GCI | 13-GC- | 62 | WCAU | Prev | 6 | 40 | 6 | N | — | Y | 0 | 160 | 27 | Surg | | 2004 |
| | | Adeno- | | | U. | | | | | | | | | | | | |
| | | SIIA | | | | | | | | | | | | | | | |
| LC | GCI | 14-GC- | 56 | WCAU | Curr | 30 | 25 | — | Y | 1 | N | — | 180 | 36.4 | Surg | | 2001 |
| | | Adeno- | | | U. | | | | | | | | | | | | |
| | | SIIA | | | | | | | | | | | | | | | |
| LC | GCI | 15-GC- | 61 | WCAU | Curr | 30 | 36 | — | Y | 1 | N | — | 163 | 25.1 | Surg | | 2004 |
| | | Adeno- | | | U. | | | | | | | | | | | | |
| | | SIIIA | | | | | | | | | | | | | | | |
| LC | Bioch | 16-(95)- | 54 | | | | | | | | | | | | | | |
| | | BC- | | | | | | | | | | | | | | | |
| | | Adeno | | | | | | | | | | | | | | | |
| LC | Bioch | 17-(89)- | 62 | | | | | | | | | | | | | | |
| | | Bc- | | | | | | | | | | | | | | | |
| | | Adeno | | | | | | | | | | | | | | | |
| LC | Bioch | 18-(76)- | 57 | | | | | | | | | | | | | | |
| | | Bc- | | | | | | | | | | | | | | | |
| | | Adeno | | | | | | | | | | | | | | | |
| LC | Bioch | 74-(2)-Bc- | 64 | | | | | | | | | | | | | | |
| | | Adeno | | | | | | | | | | | | | | | |
| LC | Bioch | 76-(75)- | 65 | | | | | | | | | | | | | | |
| | | Bc- | | | | | | | | | | | | | | | |
| | | Adeno | | | | | | | | | | | | | | | |
| LC | Bioch | 77-(12)- | 74 | | | | | | | | | | | | | | |
| | | Bc- | | | | | | | | | | | | | | | |
| | | Adeno | | | | | | | | | | | | | | | |
| LC | Bioch | 78-(13)- | 64 | | | | | | | | | | | | | | |
| | | Bc- | | | | | | | | | | | | | | | |
| | | Adeno | | | | | | | | | | | | | | | |
| LC | Bioch | 79-(94)- | 68 | | | | | | | | | | | | | | |
| | | Bc- | | | | | | | | | | | | | | | |
| | | Adeno | | | | | | | | | | | | | | | |
| LC | Ichilov | 80-(3)-Ic- | 56 | | | | | | | | | | | | | | |
| | | Adeno | | | | | | | | | | | | | | | |
| LC | Ichilov | 81-(14)- | 68 | | | | | | | | | | | | | | |
| | | Ic-Adeno | | | | | | | | | | | | | | | |
| LC | Aster | 19-As-Sq- | 67 | CAU | Curr | 11-20 | 31-40 | | | | O | | 163 | 28.6 | Surg | | 2003 |
| | | S0 | | | U. | | | | | | | | | | | | |
| LC | GCI | 20-GC- | 68 | WCAU | Prev | 10 | 20 | 0 | N | — | N | — | 157 | 22.9 | Surg | | 2004 |
| | | Sq-SIA | | | U. | | | | | | | | | | | | |
| LC | GCI | 21-GC- | 62 | WCAU | Prev | 20 | 50 | 0 | Y | 5 | N | — | 175 | 25.5 | Surg | | 2005 |
| | | Sq-SIB | | | U. | | | | | | | | | | | | |
| LC | Aster | 22-As-Sq- | 73 | CAU | Prev | | | | | | O | | 170 | 22.1 | Surg | | 2004 |
| | | SIB | | | U. | | | | | | | | | | | | |
| LC | Aster | 23-As-Sq- | 66 | CAU | Prev | 11-20 | 45 | | | | P | | 178 | 33.8 | Surg | | 2005 |
| | | SIB | | | U. | | | | | | | | | | | | |
| LC | Aster | 24-As-Sq- | 65 | CAU | Curr | 6-10 | 41-50 | | | | O | | 176 | 22 | Surg | | 2002 |
| | | SIB | | | U. | | | | | | | | | | | | |
| LC | GCI | 25-GC- | 44 | WCAU | Prev | 20 | 20 | 0 | Y | 2 | N | — | 155 | 22.7 | Surg | | 2004 |
| | | Sq-SIIB | | | U. | | | | | | | | | | | | |
| LC | GCI | 26-GC- | 68 | WCAU | Prev | 40 | 40 | 0 | Y | 2 | N | — | 170 | 23.2 | Surg | | 2004 |
| | | Sq-SIIB | | | U. | | | | | | | | | | | | |
| LC | GCI | 27-GC- | 58 | WCAU | Prev | 50 | 40 | 1 | Y | 2 | N | — | 173 | 27.4 | Surg | | 2004 |
| | | Sq-SIIIA | | | U. | | | | | | | | | | | | |
| LC | Ichilov | 28-(23)- | 65 | | | | | | | | | | | | | | |
| | | Ic-Sq | | | | | | | | | | | | | | | |
| LC | Ichilov | 29-(25)- | 72 | | | | | | | | | | | | | | |
| | | Ic-Sq | | | | | | | | | | | | | | | |
| LC | Bioch | 30-(19)- | 78 | | | | | | | | | | | | | | |
| | | Bc-Sq | | | | | | | | | | | | | | | |
| LC | Bioch | 31-(78)- | 62 | | | | | | | | | | | | | | |
| | | Bc-Sq | | | | | | | | | | | | | | | |
| LC | Bioch | 32-(16)- | 68 | | | | | | | | | | | | | | |
| | | Bc-Sq | | | | | | | | | | | | | | | |
| LC | Bioch | 33-(80)- | 74 | | | | | | | | | | | | | | |
| | | Bc-Sq | | | | | | | | | | | | | | | |
| LC | Bioch | 34-(18)- | 63 | | | | | | | | | | | | | | |
| | | Bc-Sq | | | | | | | | | | | | | | | |
| LC | Bioch | 92-(38)- | 58 | | | | | | | | | | | | | | |
| | | Bc-LCC | | | | | | | | | | | | | | | |
| LC | Bioch | 93-(82)- | 68 | | | | | | | | | | | | | | |
| | | Bc-LCC | | | | | | | | | | | | | | | |
| LC | GCI | 42-GC- | 62 | WCAU | Prev | 20 | 35 | 0.15 | Y | 2 | N | — | 165 | 19.8 | Surg | | 2003 |
| | | SCC-SIB | | | U. | | | | | | | | | | | | |
| LC | Bioch | 43-(32)- | 30 | | | | | | | | | | | | | | |
| | | Bc-SCC | | | | | | | | | | | | | | | |
| LC | Bioch | 44-(30)- | 34 | | | | | | | | | | | | | | |
| | | Bc-SCC | | | | | | | | | | | | | | | |
| LC | Bioch | 45-(83)- | 47 | | | | | | | | | | | | | | |
| | | Bc-SCC | | | | | | | | | | | | | | | |
| LC | Bioch | 46-(86)- | 52 | | | | | | | | | | | | | | |
| | | Bc-SCC | | | | | | | | | | | | | | | |
| LC | Bioch | 47-(31)- | 59 | | | | | | | | | | | | | | |
| | | Bc-SCC | | | | | | | | | | | | | | | |
| LC | Bioch | 48-(84)- | 59 | | | | | | | | | | | | | | |
| | | Bc-SCC | | | | | | | | | | | | | | | |
| LC | Bioch | 49-(85)- | 66 | | | | | | | | | | | | | | |
| | | Bc-SCC | | | | | | | | | | | | | | | |
| LC | Bioch | 50-(33)- | | | | | | | | | | | | | | | |
| | | Bc-SCC | | | | | | | | | | | | | | | |
| LN | Aster | 51-As-N- | 22 | CAU | Nev | | | | | | NU | | 0 | 0 | Surg | | 2003 |
| | | PS | | | U. | | | | | | | | | | | | |
| LN | Aster | 52-As-N- | 26 | CAU | Nev | | | | | | O | | 170 | 22.1 | Aut | CA | 2003 |
| | | PM | | | U. | | | | | | | | | | | | |
| LN | Aster | 53-As-N- | 37 | CAU | Nev | | | | | | C | | 183 | 20.9 | Aut | MCE | 2002 |
| | | PM | | | U. | | | | | | | | | | | | |
| LN | Aster | 54-As-N- | 76 | CAU | Prev | | | | | | | | 165 | 26.8 | Aut | CPul A | 2002 |
| | | PM | | | U. | | | | | | | | | | | | |
| LN | Aster | 55-As-N- | 81 | CAU | Prev | 41 or | 31-40 | | | | O | | 183 | 30.5 | Aut | CA | 2003 |
| | | PM | | | U. | more | | | | | | | | | | | |
| LC | Bioch | 35-(81)- | 53 | | | | | | | | | | | | | | |
| | | Bc-Sq | | | | | | | | | | | | | | | |
| LC | Bioch | 82-(21)- | 52 | | | | | | | | | | | | | | |
| | | Bc-Sq | | | | | | | | | | | | | | | |
| LC | Bioch | 83-(17)- | 57 | | | | | | | | | | | | | | |
| | | Bc-Sq | | | | | | | | | | | | | | | |
| LC | Bioch | 84-(79)- | 67 | | | | | | | | | | | | | | |
| | | Bc-Sq | | | | | | | | | | | | | | | |
| LC | Bioch | 85-(22)- | 48 | | | | | | | | | | | | | | |
| | | Bc-Sq | | | | | | | | | | | | | | | |
| LC | Bioch | 86-(20)- | 64 | | | | | | | | | | | | | | |
| | | Bc-Sq | | | | | | | | | | | | | | | |
| LC | Bioch | 87-(88)- | 64 | | | | | | | | | | | | | | |
| | | Bc-Sq | | | | | | | | | | | | | | | |
| LC | Bioch | 88-(100)- | 64 | | | | | | | | | | | | | | |
| | | Bc-Sq | | | | | | | | | | | | | | | |
| LC | Ichilov | 89-(24)- | 76 | | | | | | | | | | | | | | |
| | | Ic-Sq | | | | | | | | | | | | | | | |
| LC | GCI | 36-GC- | 45 | WCAU | Prev | 45 | 33 | 0 | Y | 2 | Y | 28 | 178 | 31.9 | Surg | | 2004 |
| | | LCC-SIA | | | U. | | | | | | | | | | | | |
| LC | GCI | 37-GC- | 60 | WCAU | Prev | 30 | 45 | 0 | Y | 3 | N | — | 160 | 16.8 | Surg | | 2004 |
| | | LCC-SIB | | | U. | | | | | | | | | | | | |
| LC | GCI | 38-GC- | 68 | WCAU | Prev | — | 55 | — | Y | — | N | — | 173 | 22.8 | Surg | | 2001 |
| | | LCC-SIB | | | U. | | | | | | | | | | | | |
| LC | GCI | 39-GC- | 51 | WCAU | Prev | 20 | 12 | 22 | Y | 1 | N | — | 183 | 26.6 | Surg | | 2004 |
| | | LCC-SIIB | | | U. | | | | | | | | | | | | |
| LC | GCI | 40-GC- | 62 | WCAU | Prev | 40 | 40 | 0 | Y | 2 | Y | 12 | 185 | 23.1 | Surg | | 2004 |
| | | LCC-SIIB | | | U. | | | | | | | | | | | | |
| LC | GCI | 41-GC- | 70 | WCAU | Prev | 30 | 50 | — | Y | 2 | Y | 13 | 168 | 20.7 | Surg | | 2002 |
| | | LCC-SIIB | | | U. | | | | | | | | | | | | |
| LC | Bioch | 90-(39)- | 35 | | | | | | | | | | | | | | |
| | | Bc-LCC | | | | | | | | | | | | | | | |
| LC | Bioch | 91-(87)- | 47 | | | | | | | | | | | | | | |
| | | Bc-LCC | | | | | | | | | | | | | | | |
| LN | Aster | 56-As-N- | 0 | CAU | Prev | 21-40 | 41-50 | | | | P | | 175 | 25.1 | Aut | IC | |
| | | PM | | | U. | | | | | | | | | | | | |
| LN | Aster | 57-As-N- | 69 | CAU | Curr | 21-40 | 31-50 | | | | P | | 165 | 22.4 | Aut | CPul A | 2005 |
| | | PM | | | U. | | | | | | | | | | | | |
| LN | Aster | 58-As-N- | 75 | CAU | | | | | | | | | 160 | 21.5 | Aut | CPul A | 2004 |
| | | PM | | | | | | | | | | | | | | | |
| LN | Aster | 59-As-N- | 75 | CAU | | | | | | | | | 175 | 32.7 | Aut | Cer A | 2004 |
| | | PM | | | | | | | | | | | | | | | |
| LN | Amb | 60-(99)- | 31 | | | | | | | | | | | | | | |
| | | Am-N | | | | | | | | | | | | | | | |
| | | PM | | | | | | | | | | | | | | | |
| LN | Amb | 61-(96)- | 43 | | | | | | | | | | | | | | |
| | | Am-N | | | | | | | | | | | | | | | |
| | | PM | | | | | | | | | | | | | | | |
| LN | Amb | 62-(97)- | 46 | | | | | | | | | | | | | | |
| | | Am-N | | | | | | | | | | | | | | | |
| | | PM | | | | | | | | | | | | | | | |
| LN | Amb | 63-(93)- | 61 | | | | | | | | | | | | | | |
| | | Am-N | | | | | | | | | | | | | | | |
| | | PM | | | | | | | | | | | | | | | |
| LN | Amb | 64-(98)- | 72 | | | | | | | | | | | | | | |
| | | Am-N | | | | | | | | | | | | | | | |
| | | PM | | | | | | | | | | | | | | | |
| LN | Bioch | 67-(50)- | 28 | | | | | | | | | | | | | | |
| | | Bc-N PM | | | | | | | | | | | | | | | |
| LN | Bioch | 68-(92)- | 28 | | | | | | | | | | | | | | |
| | | Bc-N PM | | | | | | | | | | | | | | | |
| LN | Bioch | 69-(91)- | 24, 29 | | | | | | | | | | | | | | |
| | | Bc-N PM | | | | | | | | | | | | | | | |
| LN | Bioch | 70-(90)- | 27, 28 | | | | | | | | | | | | | | |
| | | Bc-N PM | | | | | | | | | | | | | | | |
| LN | Bioch | 71-(48)- | 44 | | | | | | | | | | | | | | |
| | | Bc-N PM | | | | | | | | | | | | | | | |

TABLE 1_5_1

| Key | Full Name |
|---|---|
| # Cig. Per day | Number of Cigarettes per day |
| # Dr | Number of Drinks |
| # of Y. Use of Tobacco | Number of Years Using Tobacco |
| # Y. off Tobacco | Number of Years Off Tobacco |
| AC | Alveolus carcinoma |
| Aden | ADENOCARCINOMA |
| Amb | Ambion |
| Aster | Asterand |
| Aut | Autopsy |
| BC | BRONCHIOLOALVEOLAR CARCINOMA |
| Bioch | Biochain |
| C | Current Use |
| CA | Cardiac arrest |
| CAU | Caucasian |
| Cer A | Cerebrovascular accident |
| CPul A | Cardiopulmonary arrest |
| CS | Cancer Stage |
| Curr U. | Current Use |
| Diag | Diagnosis |
| Dr Al | Drink Alcohol? |
| Exc Y. | Excision Year |
| Gen | Gender |
| Gr | Grade |
| Height | HT |
| IC | Ischemic cardiomyopathy |
| LC | Lung Cancer |
| LCC | LARGE CELL CARCINOMA |
| LCNC | Large Cell Neuroendocrine Carcinoma |
| LN | Lung Normal |
| MCE | Massive cerebral edema |
| N | No |
| NC | NEUROENDOCRINE CARCINOMA |
| Nev. U. | Never Used |
| Norm-L | Normal Lung |
| N-P2-PM | Normal (Pool 2)-PM |
| N-PM | Normal-PM |
| NSCC . . . | NON-SMALL CELL CARCINOMA WITH |
| | SARCOMUTOUS TRANSFORMTAIO |
| NU | Never used |
| O | Occasional Use |
| P | Previous Use |
| P2 | Pool 2 |
| Prev U. | Previous Use |
| SCC | Squamous Cell Carcinoma |
| Sm P Y? | Have people at home smoked in past 15 yr |
| Sm ppl | If yes, how many? |
| SMCC | SMALL CELL CARCINOMA |
| SMOKE_GROWING_UP | Did people smoke at home while growing up |
| Surg | Surgical |
| Tum % | Tumor Percentage |
| WCAU | White Caucasian |
| Y | Yes |

TABLE 1_6
Tissue samples in normal panel:

| | Lot no. | Source | Tissue | Pathology | Sex/Age |
|---|---|---|---|---|---|
| 1-Am-Colon (C71) | 071P10B | Ambion | Colon | PM IC bleed | F/43 |
| 2-B-Colon (C69) | A411078 | Biochain | Colon | PM-Pool of 10 | M(26-78) & F(53-77) |
| 3-Cl-Colon (C70) | 1110101 | Clontech | Colon | PM-Pool of 3 sudden death | M & F(20-50) |
| 4-Am-Small | 091P0201A | Ambion | Small Intestine | PM ICH | M/85 |
| Intestine | | | | | |
| 5-B-Small Intestine | A501158 | Biochain | Small Intestine | PM | M/63 |
| 6-B-Rectum | A605138 | Biochain | Rectum | PM | M/25 |
| 7-B-Rectum | A610297 | Biochain | Rectum | PM | M/24 |
| 8-B-Rectum | A610298 | Biochain | Rectum | PM | M/27 |
| 9-Am-Stomach | 110P04A | Ambion | Stomach | PM GSW | M/16 |
| 10-B-Stomach | A501159 | Biochain | Stomach | PM | M/24 |
| 11-B-Esophagus | A603814 | Biochain | Esophagus | PM | M/26 |
| 12-B-Esophagus | A603813 | Biochain | Esophagus | PM | M/41 |
| 13-Am-Pancreas | 071P25C | Ambion | Pancreas | PM MVA | F/25 |
| 14-CG-Pancreas | CG-255-2 | Ichilov | Pancreas | PM | M/75 |
| 15-B-Lung | A409363 | Biochain | Lung | PM-Pool of 5 | M(24-28) & F62 |
| 16-Am-Lung (L93) | 111P0103A | Ambion | Lung | PM ICH | F/61 |
| 17-B-Lung (L92) | A503204 | Biochain | Lung | PM | M/28 |
| 19-B-Ovary (O48) | A504087 | Biochain | Ovary | PM | F/51 |
| 20-B-Ovary (O46) | A504086 | Biochain | Ovary | PM | F/41 |
| 75-G-Ovary | L629FRV1 | GCI | Ovary | PS DIGESTIVE HEMORRHAGE | F/47 |
| | | | | (ALCOHOLISM) | |
| 76-G-Ovary | DWHTZRQX | GCI | Ovary | PS LEIOMYOMAS | F/42 |
| 77-G-Ovary | FDPL9NJ6 | GCI | Ovary | PS VAGINAL BLEEDING | F/56 |
| 78-G-Ovary | GWXUZN5M | GCI | Ovary | PS ABNORMAL PAP SMEARS | F/53 |
| 21-Am-Cervix | 101P0101A | Ambion | Cervix | PM Surgery | F/40 |
| 23-B-Cervix | A504089 | Biochain | Cervix | PM-Pool of 5 | F(36-55) |
| 24-B-Uterus | A411074 | Biochain | Uterus | PM-Pool of 10 | F(32-53) |
| 25-B-Uterus | A409248 | Biochain | Uterus | PM | F/35 |
| 26-B-Uterus | A504090 | Biochain | Uterus | PM-Pool of 5 | F(40-53) |
| 28-Am-Bladder | 071P02C | Ambion | Bladder | PM GSW | M/28 |
| 29-B-Bladder | A504088 | Biochain | Bladder | PM-Pool of 5 | M(26-44) & F30 |
| 30-Am-Placenta | 021P33A | Ambion | Placenta | PB | F/33 |
| 31-B-Placenta | A410165 | Biochain | Placenta | PB | F/26 |
| 32-B-Placenta | A411073 | Biochain | Placenta | PB-Pool of 5 | F(24-30) |
| 33-B-Breast (B59) | A607155 | Biochain | Breast | PM | F/36 |
| 34-Am-Breast (B63) | 26486 | Ambion | Breast | PS bilateral breast reduction | F/43 |
| 35-Am-Breast (B64) | 23036 | Ambion | Breast | PM lung cancer | F/57 |
| 36-Cl-Prostate (P53) | 1070317 | Clontech | Prostate | PM-Pool of 47 sudden death | M(14-57) |
| 37-Am-Prostate | 061P04A | Ambion | Prostate | PM IC bleed | M/47 |
| (P42) | | | | | |
| 38-Am-Prostate | 25955 | Ambion | Prostate | PM head trauma | M/62 |
| (P59) | | | | | |
| 39-Am-Testis | 111P0104A | Ambion | Testis | PM GSW | M/25 |
| 40-B-Testis | A411147 | Biochain | Testis | PM | M/74 |
| 41-Cl-Testis | 1110320 | Clontech | Testis | PM-Pool of 45 sudden death | M(14-64) |
| 42-CG-Adrenal | CG-184-10 | Ichilov | Adrenal | PM | F/81 |
| 43-B-Adrenal | A610374 | Biochain | Adrenal | PM | F/83 |
| 44-B-Heart | A411077 | Biochain | Heart | PM-Pool of 5 | M(23-70) |
| 45-CG-Heart | CG-255-9 | Ichilov | Heart focal | PM | M/75 |
| | | | fibrosis | | |
| 46-CG-Heart | CG-227-1 | Ichilov | Heart | PM | F/36 |
| 47-Am-Liver | 081P0101A | Ambion | Liver | PM ICH | M/64 |
| 48-CG-Liver | CG-93-3 | Ichilov | Liver | PM | F/19 |
| 49-CG-Liver | CG-124-4 | Ichilov | Liver of fetus | PM | fetus |
| 50-Cl-BM | 1110932 | Clontech | Bone Marrow | PM-Pool of 8 sudden death | M & F(22-65) |
| 51-CGEN-Blood | WBC#5 | CGEN | Blood | — | M |
| 52-CGEN-Blood | WBC#4 | CGEN | Blood | — | M |
| 53-CGEN-Blood | WBC#3 | CGEN | Blood | — | M |
| 54-CG-Spleen | CG-267 | Ichilov | Spleen | PM | F/25 |
| 55-CG-Spleen | 111P0106B | Ambion | Spleen | PM GSW | M/25 |
| 56-CG-Spleen | A409246 | Biochain | Spleen | PM | F/12 |
| 57-CG-Thymus | CG-98-7 | Ichilov | Thymus | PM | F/28 |
| 58-Am-Thymus | 101P0101A | Ambion | Thymus | PM head injury | M/14 |
| 59-B-Thymus | A409278 | Biochain | Thymus | PM | M/28 |
| 60-B-Thyroid | A610287 | Biochain | Thyroid | PM | M/27 |
| 61-B-Thyroid | A610286 | Biochain | Thyroid | PM | M/24 |
| 62-CG-Thyroid | CG-119-2 | Ichilov | Thyroid | PM | F/66 |
| 63-Cl-Salivary | 1070319 | Clontech | Salivary Gland | PM-Pool of 24 sudden death | M & F 15-60 |
| Gland | | | | | |
| 64-Am-Kidney | 111P0101B | Ambion | Kidney | PM ICH | M 60 |
| 65-Cl-Kidney | 1110970 | Clontech | Kidney | PM-Pool of 14 sudden death | M & F 18-59 |
| 66-B-Kidney | A411080 | Biochain | Kidney | PM-Pool of 5 | M24-46 |
| 67-CG-Cerebellum | CG-183-5 | Ichilov | Cerebellum | PM | M/74 |
| 68-CG-Cerebellum | CG-212-5 | Ichilov | Cerebellum | PM | M/54 |
| 69-B-Brain | A411322 | Biochain | Brain | PM | M/28 |
| 70-Cl-Brain | 1120022 | Clontech | Brain | PM | — |
| 71-B-Brain | A411079 | Biochain | Brain | PM-Pool of 2 | M27-28 |
| 72-CG-Brain | CG-151-1 | Ichilov | Brain | PM | F/86 |
| 73-Am-Skeletal | 101P013A | Ambion | Skeletal Muscle | PM head injury | F/28 |
| Muscle | | | | | |
| 74-Cl-Skeletal | 1061038 | Clontech | Skeletal Muscle | PM-Pool of 2 sudden death | M & F 43-46 |
| Muscle | | | | | |

TABLE 1_7

| | | Sample id(GCI)/case id | Tissue id (GCI)/Specimen | Sample id (Asterand)/RNA id |
|---|---|---|---|---|
| sample name | Source | (Asterand) Lot no. | id (Asternd) | (GCI) |
| 1-(7)-Bc-Rectum | Biochain | A610297 | | |
| 2-(8)-Bc-Rectum | Biochain | A610298 | | |
| 3-GC-Colon | GCI | CDSUV | CDSUVNR3 | |
| 4-As-Colon | Asterand | 16364 | 31802 | 31802B1 |
| 5-As-Colon | Asterand | 22900 | 74446 | 74446B1 |
| 6-GC-Small bowl | GCI | V9L7D | V9L7DN6Z | |
| 7-GC-Small bowl | GCI | M3GVT | M3GVTN5R | |
| 8-GC-Small bowl | GCI | 196S2 | 196S2AJN | |
| 9-(9)-Am-Stomach | Ambion | 110P04A | | |
| 10-(10)-Bc-Stomach | Biochain | A501159 | | |
| 11-(11)-Bc-Esoph | Biochain | A603814 | | |
| 12-(12)-Bc-Esoph | Biochain | A603813 | | |
| 13-As-Panc | Asterand | 8918 | 9442 | 9442C1 |
| 14-As-Panc | Asterand | 10082 | 11134 | 11134B1 |
| 15-(48)-Ic-Liver | Ichilov | CG-93-3 | | |
| 16-As-Liver | Asterand | 7916 | 7203 | 7203B1 |
| 17-(28)-Am-Bladder | Ambion | 071P02C | | |
| 18-(29)-Bc-Bladder | Biochain | A504088 | | |
| 19-(64)-Am-Kidney | Ambion | 111P0101B | | |
| 20-(65)-Cl-Kidney | Clontech | 1110970 | | |
| 21-(66)-Bc-Kidney | Biochain | A411080 | | |
| 22-GC-Kidney | GCI | N1EVZ | N1EVZN91 | |
| 23-GC-Kidney | GCI | BMI6W | BMI6WN9F | |
| 24-(42)-Ic-Adrenal | Ichilov | CG-184-10 | | |
| 25-(43)-Bc-Adrenal | Biochain | A610374 | | |
| 26-(16)-Am-Lung | Ambion | 111P0103A | | |
| 27-(17)-Bc-Lung | Biochain | A503204 | | |
| 28-As-Lung | Asterand | 9078 | 9275 | 9275B1 |
| 29-As-Lung | Asterand | 6692 | 6161 | 6161A1 |
| 30-As-Lung | Asterand | 7900 | 7180 | 7180F1 |
| 31-(75)-GC-Ovary | GCI | L629FRV1 | | |
| 32-(76)-GC-Ovary | GCI | DWHTZRQX | | |
| 33-(77)-GC-Ovary | GCI | FDPL9NJ6 | | |
| 34-(78)-GC-Ovary | GCI | GWXUZN5M | | |
| 35-(21)-Am-Cerix | Ambion | 101P0101A | | |
| 36-GC-cervix | GCI | E2P2N | E2P2NAP4 | |
| 37-(24)-Bc-Uterus | Biochain | A411074 | | |
| 38-(26)-Bc-Uterus | Biochain | A504090 | | |
| 39-(30)-Am-Placen | Ambion | 021P33A | | |
| 40-(32)-Bc-Placen | Biochain | A411073 | | |
| 41-GC-Breast | GCI | DHLR1 | | |
| 42-GC-Breast | GCI | TG6J6 | | |
| 43-GC-Breast | GCI | E6UDD | E6UDDNCF | |
| 44-(38)-Am-Prostate | Ambion | 25955 | | |
| 45-Bc-Prostate | Biochain | A609258 | | |
| 46-As-Testis | Asterand | 13071 | 19567 | 19567B1 |
| 47-As-Testis | Asterand | 19671 | 42120 | 42120A1 |
| 48-GC-Artery | GCI | 7FUUP | 7FUUPAMP | |
| 49-GC-Artery | GCI | YGTVY | YGTVYAIN | |
| 50-Th-Blood-PBMC | Tel- | 52497 | | |
| | Hashomer | | | |
| 51-Th-Blood-PBMC | Tel- | 31055 | | |
| | Hashomer | | | |
| 52-Th-Blood-PBMC | Tel- | 31058 | | |
| | Hashomer | | | |
| 53-(54)-Ic-Spleen | Ichilov | CG-267 | | |
| 54-(55)-Ic-Spleen | Ichilov | 111P0106B | | |
| 55-(57)-Ic-Thymus | Ichilov | CG-98-7 | | |
| 56-(58)-Am-Thymus | Ambion | 101P0101A | | |
| 57-(60)-Bc-Thyroid | Biochain | A610287 | | |
| 58-(62)-Ic-Thyroid | Ichilov | CG-119-2 | | |
| 59-Gc-Sali gland | GCI | NNSMV | NNSMVNJC | |
| 60-(67)-Ic-Cerebellum | Ichilov | CG-183-5 | | |
| 61-(68)-Ic-Cerebellum | Ichilov | CG-212-5 | | |
| 62-(69)-Bc-Brain | Biochain | A411322 | | |
| 63-(71)-Bc-Brain | Biochain | A411079 | | |
| 64-(72)-Ic-Brain | Ichilov | CG-151-1 | | |
| 65-(44)-Bc-Heart | Biochain | A411077 | | |
| 66-(46)-Ic-Heart | Ichilov | CG-227-1 | | |
| 67-(45)-Ic-Heart | Ichilov | CG-255-9 | | |
| (Fibrotic) | | | | |
| 68-GC-Skel Mus | GCI | T8YZS | T8YZSN7O | |
| 69-GC-Skel Mus | GCI | Q3WKA | Q3WKANCJ | |
| 70-As-Skel Mus | Asterand | 8774 | 8235 | 8235G1 |
| 71-As-Skel Mus | Asterand | 8775 | 8244 | 8244A1 |
| 72-As-Skel Mus | Asterand | 10937 | 12648 | 12648C1 |
| 73-As-Skel Mus | Asterand | 6692 | 6166 | 6166A1 |

TABLE 1_8
Breast panel for MA analysis

| | | | | | |
|---|---|---|---|---|---|
| Sample RT # | MA-TAA | sample rename | Lot no | source | pathology |

Cancer

| | | | | | |
|---|---|---|---|---|---|
| RT-1 | BreCa-1 | 14-A-IDC G2 | A0135T | ABS | IDC |
| RT-2 | BreCa-2 | 43-B-IDC G2 | A609183 | Biochain | IDC |
| RT-3 | BreCa-3 | 54-B-IDC G2 | A605353 | Biochain | IDC |
| RT-4 | BreCa-4 | 55-B-IDC G2 | A609179 | Biochain | IDC |
| RT-5 | BreCa-5 | 17-A-IDC G2 | 4904020036T | ABS | IDC |
| RT-6 | BreCa-6 | 42-A-IDC G3 | 6005020031T | ABS | IDC |
| RT-7 | BreCa-7 | 7-A-IDC G2 | 7263T | ABS | IDC |
| RT-8 | BreCa-8 | 48-B-IDC G2 | A609222 | Biochain | IDC |
| RT-9 | BreCa-9 | 12-A-IDC G2 | 1432T | ABS | IDC |
| RT-10 | BreCa-10 | 46-B-Carci G2 | A609177 | Biochain | Carcinoma |
| RT-11 | BreCa-11 | 16-A-IDC G2 | 4904020032T | ABS | IDC |
| RT-12 | BreCa-12 | 49-B-IDC G2 | A609223 | Biochain | IDC |
| RT-13 | BreCa-13 | 32-A-Muc Carci | 7116T | ABS | Mucinous |
| RT-14 | BreCa-14 | 45-B-IDC G2 | A609181 | Biochain | IDC |
| RT-15 | BreCa-15 | 15-A-IDC G2 | 7259T | ABS | IDC |
| RT-16 | BreCa-16 | 6-A-IDC G1 | 7238T | ABS | IDC |
| RT-17 | BreCa-17 | 26-A-IDC G3 | 7249T | ABS | IDC |
| RT-18 | BreCa-18 | 13-A-IDC G2 | A0133T | ABS | IDC |
| RT-19 | BreCa-19 | 50-B-IDC G2 | A609224 | Biochain | IDC |
| RT-20 | BreCa-20 | 44-B-IDC G2 | A609198 | Biochain | IDC |
| RT-21 | BreCa-21 | 51-B-IDC G1 | A605361 | Biochain | IDC |
| RT-22 | BreCa-22 | 27-A-IDC G3 | 4907020072T | ABS | IDC |
| RT-23 | BreCa-23 | 3Z5Z4ANH | 3Z5Z4RVE | GCI | IDC |
| RT-24 | BreCa-24 | 4W2NYAC1 | 4W2NYR9S | GCI | IDC |
| RT-25 | BreCa-25 | 54NTAAKT | 54NTAR75 | GCI | IDC |
| RT-26 | BreCa-26 | I2YLEACP | I2YLERVY | GCI | IDC |
| RT-27 | BreCa-27 | J5MPNA9Q | J5MPNRQI | GCI | IDC |
| RT-28 | BreCa-28 | KIOE7AI9 | KIOE7RWK | GCI | IDC |
| RT-29 | BreCa-29 | OLKL4AO6 | OLKL4RZ9 | GCI | IDC |
| RT-30 | BreCa-30 | RD3F9AFQ | RD3F9RY9 | GCI | IDC |
| RT-31 | BreCa-31 | SE5BKAEQ | SE5BKRHY | GCI | IDC |
| RT-32 | BreCa-32 | VK1EJAQE | VK1EJRKH | GCI | IDC |
| RT-33 | BreCa-33 | YOLOFARG | YOLOFRE7 | GCI | IDC |
| RT-34 | BreCa-34 | YQ1WWAUV | YQ1WWROR | GCI | IDC |
| RT-35 | BreCa-35 | YSZ67A48 | YSZ67ROA | GCI | IDC |
| RT-36 | BreCa-36 | POPHPAZ4 | POPHPRDM | GCI | IDC |
| RT-37 | BreCa-37 | 5IRTKAXT | 5IRTKRTG | GCI | IDC |
| RT-38 | BreCa-38 | DSI52AH3 | DSI52RVW | GCI | IDC |
| RT-39 | BreCa-39 | GETCVAY2 | GETCVRIT | GCI | IDC |
| RT-40 | BreCa-40 | S2GBYAGC | S2GBYRR1 | GCI | IDC |
| RT-41 | BreCa-41 | UT3SEAQY | UT3SERM8 | GCI | IDC |
| RT-42 | BreCa-42 | PVSYXA72 | PVSYXR66 | GCI | IDC |
| RT-43 | BreCa-43 | 17138 | 30697A1 | Asterand | IDC |
| RT-44 | BreCa-44 | 17959 | 31225A1 | Asterand | IDC |
| RT-45 | BreCa-45 | 52-B-ILC G1 | A605360 | Biochain | ILC |
| RT-46 | BreCa-46 | IS84YAAY | IS84YR6E | GCI | ILC |
| RT-47 | BreCa-47 | I35USA9G | I35USR7K | GCI | ILC |
| RT-48 | BreCa-48 | 17090 | 30738A1 | Asterand | ILC |
| RT-49 | BreCa-49 | 42509 | 42509A1 | Asterand | Ductal Carcinoma In |
| | | | | | Situ(DCIS) |

Benign

| | | | | | |
|---|---|---|---|---|---|
| RT-50 | BreBe-1 | NNP3QA4V | NNP3QRCW | GCI | FIBROADENOMA OF THE |
| | | | | | BREAST |
| RT-51 | BreBe-2 | QK8IYALU | QK8IYRW1 | GCI | FIBROADENOMA OF THE |
| | | | | | BREAST |
| RT-52 | BreBe-3 | ZT15MAMR | ZT15MR2Y | GCI | FIBROADENOMA OF THE |
| | | | | | BREAST |
| RT-53 | BreBe-4 | 11975 | 15478B1 | Asterand | Fibroadenoma |

Normal

| | | | | | |
|---|---|---|---|---|---|
| RT-54 | BreNo-1 | 57-B-N | A609233 | Biochain | Normal post mortem |
| RT-55 | BreNo-2 | 59-B-N | A607155 | Biochain | Normal post mortem |
| RT-56 | BreNo-3 | 60-B-N | A609234 | Biochain | Normal post mortem |
| RT-57 | BreNo-4 | 63-Am-N | 26486 | Ambion | Normal post surgery |
| RT-58 | BreNo-5 | 66-Am-N | 36678 | Ambion | Normal post mortem |
| RT-59 | BreNo-6 | 64-Am-N | 23036 | Ambion | Normal post mortem |
| RT-60 | BreNo-7 | 56-B-N | A609235 | Biochain | Normal post mortem |
| RT-61 | BreNo-8 | 65-Am-N | 31410 | Ambion | Normal post mortem |
| RT-62 | BreNo-9 | 67-Am-N | 073P010602086A | Ambion | Normal post mortem |
| RT-63 | BreNo-10 | 58-B-N | A609232 | Biochain | Normal post mortem |
| RT-64 | BreNo-11 | DHLR1NIQ | DHLR1R8J | GCI | Normal post surgery |
| RT-65 | BreNo-12 | 14398 | 20021D1 | Asterand | Normal post surgery |

TABLE 1_9
Ovary panel for MA analysis

| | | | | |
|---|---|---|---|---|
| | TAA2_MA | | | |
| Sample # | ID | Tissue ID | RNA ID | Source |

OvSr = SEROUS
ADENOCARCINOMA

| | | | | |
|---|---|---|---|---|
| 1 | OvSr1 | 2O37OAI3 | 2O37ORTX | GCI |
| 2 | OvSr2 | 3NTISA77 | 3NTISRY4 | GCI |
| 3 | OvSr3 | 4WAABA68 | 4WAABR62 | GCI |
| 4 | OvSr4 | 79Z67AL4 | 79Z67RFA | GCI |
| 5 | OvSr5 | 7B3DPA5S | 7B3DPR3Y | GCI |
| 6 | OvSr6 | 7RMHZAMG | 7RMHZRQ9 | GCI |
| 7 | OvSr7 | CEJUSAVO | CEJUSRZG | GCI |
| 8 | OvSr8 | DDSNLAWD | DDSNLR79 | GCI |
| 9 | OvSr9 | DH8PHAMR | DH8PHRPE | GCI |
| 10 | OvSr10 | 5NCLKA15 | 5NCLKR2O | GCI |
| 11 | OvSr11 | 1HI5HAHH | 1HI5HRE2 | GCI |
| 12 | OvSr12 | 33-B-Pap Sero CystAde G1 | | BioChain |
| 13 | OvSr13 | 31-B-Pap Sero CystAde G3 | | BioChain |
| 14 | OvSr14 | 29-G-Sero Adeno G3 | G035 | GOG |
| 15 | OvSr15 | 9-G-Adeno G3 | 99-06-G901 | GOG |
| 66 | OvSr16 | 18701 | 40773C1 | Asterand |
| 67 | OvSr17 | 13268 | 19832A1 | Asterand |

OvPp = Papillary adenocarcinoma

| | | | | |
|---|---|---|---|---|
| 16 | OvPp1 | 4-A-Pap CystAdeno G2 | ILS-7286 | ABS |
| 17 | OvPp2 | 3-A-Pap Adeno G2 | ILS-1431 | ABS |
| 18 | OvPp3 | 2-A-Pap Adeno G2 | ILS-1408 | ABS |
| 19 | OvPp4 | 25-A-Pap Sero Adeno G3 | N0021 | ABS |
| 20 | OvPp5 | 1-A-Pap Adeno G3 | ILS-1406 | ABS |
| 21 | OvPp6 | 66-G-Pap Sero Adeno G3 SIV | 2000-01- | GOG |
| | | | G413 | |

OvEm = ENDOMETROID
ADENOCARINOMA

| | | | | |
|---|---|---|---|---|
| 22 | OvEm1 | 1U52XAHJ | 1U52XRPE | GCI |
| 23 | OvEm2 | 533DXAHE | 533DXRKV | GCI |
| 24 | OvEm3 | 5895CAXD | 5895CR56 | GCI |
| 25 | OvEm4 | A17WSACA | A17WSR7Y | GCI |
| 26 | OvEm5 | E2WKFA4F | E2WKFRPT | GCI |
| 27 | OvEm6 | HZ2EYAU6 | HZ2EYRC6 | GCI |
| 28 | OvEm7 | PZQXHALS | PZQXHRGN | GCI |
| 29 | OvEm8 | RWOIVALL | RWOIVRI1 | GCI |
| 30 | OvEm9 | 1VT3IAZ6 | 1VT3IRT1 | GCI |
| 31 | OvEm10 | I8VHZALI | I8VHZRR4 | GCI |
| 32 | OvEm11 | 34-G-Pap Endo Adeno G3 | 95-04-2002 | GOG |

OvMu = Mucinous adenocarcinoma

| | | | | |
|---|---|---|---|---|
| 33 | OvMu1 | 22-A-Muc CystAde G2 | A0139 | ABS |
| 34 | OvMu4 | 23-A-Muc CystAde G3 | VNM-00187 | ABS |
| 35 | OvMu6 | 19-B-Muc Adeno G3 | A504085 | BioChain |
| 36 | OvMu3 | 17-B-Muc Adeno G3 | A504084 | BioChain |
| 37 | OvMu5 | IMDA1ANG | IMDA1RQG | GCI |
| 38 | OvMu2 | 21-G-Muc CystAde G2-3 | 95-10-G020 | GOG |
| 68 | OvMu7 | 12742 | 18920A1 | Asterand |
| 69 | OvMu8 | NJM4UAC4 | NJM4URI5 | GCI |
| 70 | OvMu9_BL | 3D5FOA9R | 3D5FORJ9 | GCI |
| 71 | OvMu10_BL | 7JP3FAIH | 7JP3FRCY | GCI |
| 72 | OvMu11_BL | SC656AKT | SC656RN6 | GCI |

OvBe = Benign samples

| | | | | |
|---|---|---|---|---|
| 39 | OvBe1 | 62-G-Ben Muc CysAdenoma | 99-10-G442 | GOG |
| 40 | OvBe2 | 60-G-Muc CysAdenoma | 99-01-G043 | GOG |
| 41 | OvBe3 | 56-G-Ben Muc CysAdeno | 99-01-G407 | GOG |
| 42 | OvBe4 | 64-G-Ben Sero CysAdenoma | 99-06-G039 | GOG |
| 43 | OvBe5 | 59-G-Sero CysAdenoFibroma | 98-12-G401 | GOG |
| 44 | OvBe6 | QLIKYAKS | QLIKYRNG | GCI |
| 45 | OvBe7 | 943ECATN | 943ECRVO | GCI |
| 46 | OvBe8 | 943ECAW7 | 943ECRYK | GCI |
| 47 | OvBe9 | JO8W7AKQ | JO8W7RTI | GCI |
| 48 | OvBe10 | DQQ2FAMC | DQQ2FRAC | GCI |

NOv = Normal Samples

| | | | | |
|---|---|---|---|---|
| 49 | NOv1 | 45-B-N | A503274 | BioChain |
| 50 | NOv2 | 46-B-N | A504086 | BioChain |
| 51 | NOv3 | 48-B-N | A504087 | BioChain |
| 52 | NOv4 | GWXUZN5M | GWXUZRI3 | GCI |
| 53 | NOv5 | IDUVYN9I | IDUVYROT | GCI |
| 54 | NOv6 | L629FN58 | L629FRV1 | GCI |
| 55 | NOv7 | SJ2R2NPS | SJ2R2RFN | GCI |
| 56 | NOv8 | TW9PMN69 | TW9PMR25 | GCI |
| 57 | NOv9 | XLB23NA4 | XLB23RKV | GCI |
| 58 | NOv10 | DWHTZNBF | DWHTZRQX | GCI |
| 59 | NOv11 | FDPL9NJ6 | FDPL9RVC | GCI |
| 60 | NOv12 | TOAE5N2M | TOAE5R37 | GCI |
| 61 | NOv13 | DD73BNIO | DD73BR3V | GCI |

OvExtr = Clear cell & other samples

| | | | | |
|---|---|---|---|---|
| 62 | OvExtr1 | 41-G-Mix Sero/Muc/Endo G2 | 98-03-G803 | GOG |
| 63 | OvExtr2 | 43-G-Clear cell Adeno G3 | 2001-10- | GOG |
| | | | G002 | |
| 64 | OvExtr3 | 44-G-Clear cell Adeno | 2001-07- | GOG |
| | | | G084 | |
| 65 | OvExtr4 | 42-G-Adeno borderline | 98-08-G001 | GOG |

### Materials and Experimental Procedures

RNA preparation—RNA was obtained from ABS (Wilmington, Del. 19801, USA, absbioreagents.com), BioChain Inst. Inc. (Hayward, Calif. 94545 USA biochain.com), GOG for ovary samples—Pediatric Cooperative Human Tissue Network, Gynecologic Oncology Group Tissue Bank, Children Hospital of Columbus (Columbus Ohio 43205 USA), Clontech (Franklin Lakes, N.J. USA 07417, clontech.com), Ambion (Austin, Tex. 78744 USA, ambion.com), Asternad (Detroit, Mich. 48202-3420, USA, asterand.com), and from Genomics Collaborative Inc., a Division of Seracare (Cambridge, Mass. 02139, USA, .genomicsinc.com). Alternatively, RNA was generated from tissue samples using TRI-Reagent (Molecular Research Center), according to Manufacturer's instructions. Tissue and RNA samples were obtained from patients or from postmortem. Total RNA samples were treated with DNaseI (Ambion).

RT PCR—Purified RNA (1 μg) was mixed with 150 ng Random Hexamer primers (Invitrogen) and 500 μM dNTP in a total volume of 15.6 μl. The mixture was incubated for 5 min at 65° C. and then quickly chilled on ice. Thereafter, 5 μl of 5× SuperscriptII first strand buffer (Invitrogen), 2.4 μl 0.1M DTT and 40 units RNasin (Promega) were added, and the mixture was incubated for 10 mM at 25° C., followed by further incubation at 42° C. for 2 mM. Then, 1 μl (200 units) of SuperscriptII (Invitrogen) was added and the reaction (final volume of 25 μl) was incubated for 50 min at 42° C. and then inactivated at 70° C. for 15 min. The resulting cDNA was diluted 1:20 in TE buffer (10 mM Tris pH=8, 1 mM EDTA pH=8).

Real-Time RT-PCR analysis—cDNA (5 μl), prepared as described above, was used as a template in Real-Time PCR reactions using the SYBR Green I assay (PE Applied Biosystem) with specific primers and UNG Enzyme (Eurogentech or ABI or Roche). The amplification was effected as follows: 50° C. for 2 min, 95° C. for 10 min, and then 40 cycles of 95° C. for 15 sec, followed by 60° C. for 1 min. Detection was performed by using the PE Applied Biosystem SDS 7000. The cycle in which the reactions achieved a threshold level (Ct) of fluorescence was registered and was used to calculate the relative transcript quantity in the RT reactions. Non-detected samples were assigned Ct value of 41 and were calculated accordingly. The relative quantity was calculated using the equation Q=efficiencŷ^{-Ct}. The efficiency of the PCR reaction was calculated from a standard curve, created by using serial dilutions of several reverse transcription (RT) reactions. To minimize inherent differences in the RT reaction, the resulting relative quantities were normalized to normalization factor calculated in one of the following methods as indicated in the text:

Method 1—the geometric mean of the relative quantities of the selected housekeeping (HSKP) genes was used as normalization factor.

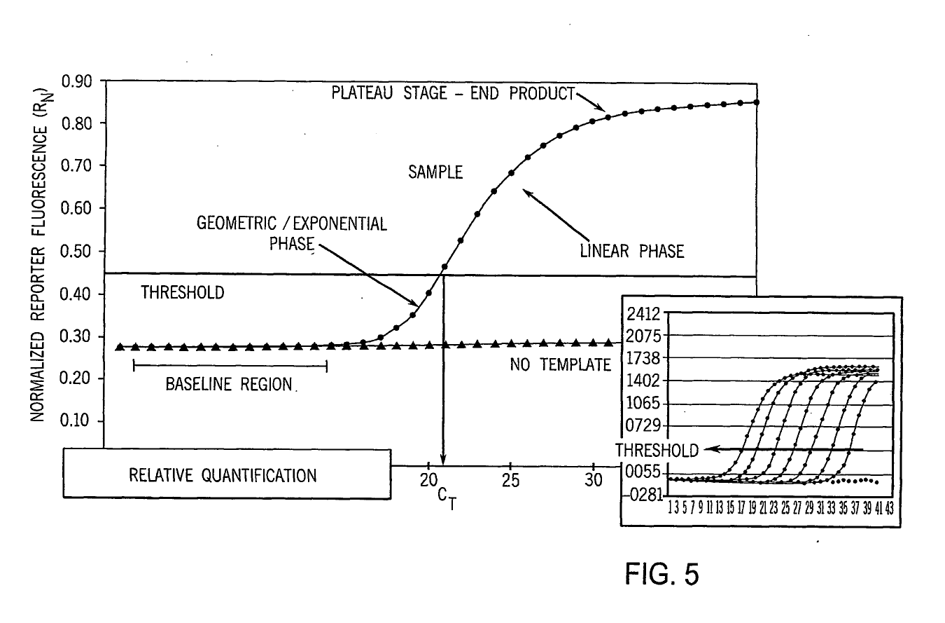

Method 2—The expression of several housekeeping (HSKP) genes was checked on every panel. The relative quantity (Q) of each housekeeping gene in each sample, calculated as described above, was divided by the median quantity of this gene in all panel samples to obtain the “relative Q rel to MED”. Then, for each sample the median of the “relative Q rel to MED” of the selected housekeeping genes was calculated and served as normalization factor of this sample for further calculations. Schematic summary of quantitative real-time PCR analysis is presented in FIG. 5. As shown, the x-axis shows the cycle number. The C_{T}=Threshold Cycle point, which is the cycle that the amplification curve crosses the fluorescence threshold that was set in the experiment. This point is a calculated cycle number in which PCR products signal is above the background level (passive dye ROX) and still in the Geometric/Exponential phase (as shown, once the level of fluorescence crosses the measurement threshold, it has a geometrically increasing phase, during which measurements are most accurate, followed by a linear phase and a plateau phase; for quantitative measurements, the latter two phases do not provide accurate measurements). The y-axis shows the normalized reporter fluorescence. It should be noted that this type of analysis provides relative quantification.

Real-Time RT-PCR analysis using TaqMan® probes—cDNA (5 μl), prepared as described above, was used as a template in Real-Time PCR reactions using the TaqMan Universal PCR Master mix (PE Applied Biosystem) with specific primers and specific TaqMan® MGB probes. The primers were used at a concentration of 500 nM and the probes at a concentration of 200 nM. The amplification was effected as follows: 50° C. for 2 min, 95° C. for 10 min, and then 40 cycles of 95° C. for 15 sec, followed by 60° C. for 1 min. Detection was performed by using the PE Applied Biosystem SDS 7000. The cycle in which the reactions achieved a threshold level (Ct) of fluorescence was registered and was used to calculate the relative transcript quantity in the RT reactions. The relative quantity was calculated using the equation Q=2̂−Ct. To minimize inherent differences in the RT reaction, the resulting relative quantities were normalized using normalization factor calculated as follows: The expression of several housekeeping (HSKP) genes was checked on the RT panel by qRT-PCR using SYBR Green detection. The relative quantity (Q) of each housekeeping gene in each sample, calculated as described above, was divided by the median quantity of this gene in all panel samples to obtain the “relative Q rel to MED”. Then, for each sample the median of the “relative Q rel to MED” of the selected housekeeping genes was calculated and served as normalization factor of this sample for further calculations. Schematic summary of quantitative real-time PCR analysis is presented in FIG. 1. As shown, the x-axis shows the cycle number. The CT=Threshold Cycle point, which is the cycle that the amplification curve crosses the fluorescence threshold that was set in the experiment. This point is a calculated cycle number in which PCR products signal is above the background level (passive dye ROX) and still in the Geometric/Exponential phase (as shown, once the level of fluorescence crosses the measurement threshold, it has a geometrically increasing phase, during which measurements are most accurate, followed by a linear phase and a plateau phase; for quantitative measurements, the latter two phases do not provide accurate measurements). The y-axis shows the normalized reporter fluorescence. It should be noted that this type of analysis provides relative quantification.

The sequences of the housekeeping genes measured in all the examples on ovarian cancer panel were as follows:

SDHA (GenBank Accession No. NM_004168 (SEQ. ID NO: 33);
SDHA Forward primer (SEQ. ID NO: 34): TGGGAACAAGAGGGCATCTG
SDHA Reverse primer (SEQ. ID NO: 35): CCACCACTGCATCAAATTCATG
SDHA-amplicon (SEQ. ID NO: 36): TGGGAACAAGAGGGCATCTGCTAAA
GTTTCAGATTCCATTTCTGCTCAGTATCCAGTAGT
GGATCATGAATTTGATGCAGTGGTGG
PBGD (GenBank Accession No. BC019323 (SEQ. ID NO: 1)),
PBGD Forward primer (SEQ. ID NO: 2): TGAGAGTGATTCGCGTGGG
PBGD Reverse primer (SEQ. ID NO: 3): CCAGGGTACGAGGCTTTCAAT
PBGD-amplicon (SEQ. ID NO: 4): TGAGAGTGATTCGCGTGGGTACCCGCAA
GAGCCAGCTTGCTCGCATACAGACGGACAGT
GTGGTGGCAACATTGAAAGCCTCGTACCCTGG
HPRT1 (GenBank Accession No. NM_000194 (SEQ. ID NO: 5)),
HPRT1 Forward primer (SEQ. ID NO: 6): TGACACTGGCAAAACAATGCA
HPRT1 Reverse primer (SEQ. ID NO: 7): GGTCCTTTTCACCAGCAAGCT
HPRT1-amplicon (SEQ. ID NO: 8): TGACACTGGCAAAACAATGCAGACTTTGC
TTTCCTTGGTCAGGCAGTATAATCCAAAGAT
GGTCAAGGTCGCAAGCTTGCTGGTGAAAAGGACC
GAPDH (GenBank Accession No. BC026907 (SEQ. ID NO: 9))
GAPDH Forward primer (SEQ. ID NO: 10): TGCACCACCAACTGCTTAGC
GAPDH Reverse primer (SEQ. ID NO: 11): CCATCACGCCACAGTTTCC
GAPDH-amplicon (SEQ. ID NO: 12): TGCACCACCAACTGCTTAGCACCCCTGG
CCAAGGTCATCCATGACAACTTTGGTATCGTG
GAAGGACTCATGACCACAGTCCATGCCATCACTGCCACCCAGAAGACTGTGGATGG

The sequences of the housekeeping genes measured in all the examples on colon cancer tissue testing panel were as follows:

PBGD (GenBank Accession No. BC019323 (SEQ. ID NO: 1)),
PBGD Forward primer (SEQ. ID NO: 2): TGAGAGTGATTCGCGTGGG
PBGD Reverse primer (SEQ. ID NO: 3): CCAGGGTACGAGGCTTTCAAT
PBGD-amplicon (SEQ. ID NO: 4): TGAGAGTGATTCGCGTGGGTACCCG
CAAGAGCCAGCTTGCTCGCATACAGACGGACAGT
GTGGTGGCAACATTGAAAGCCTCGTACCCTGG
HPRT1 (GenBank Accession No. NM_000194 (SEQ. ID NO: 5)),
HPRT1 Forward primer (SEQ. ID NO: 6): TGACACTGGCAAAACAATGCA
HPRT1 Reverse primer (SEQ. ID NO: 7): GGTCCTTTTCACCAGCAAGCT
HPRT1-amplicon (SEQ. ID NO: 8): TGACACTGGCAAAACAATGCAGACT
TTGCTTTCCTTGGTCAGGCAGTATAATCCAAAGAT
GGTCAAGGTCGCAAGCTTGCTGGTGAAAAGGACC
G6PD (GenBank Accession No. NM_000402 (SEQ. ID NO: 13))
G6PD Forward primer (SEQ. ID NO: 14): gaggccgtcaccaagaacat
G6PD Reverse primer (SEQ. ID NO: 15): ggacagccggtcagagctc
G6PD-amplicon (SEQ. ID NO: 16): gaggccgtcaccaagaacattcacgagtcctgcatgag
ccagataggctggaaccgcatcatcgtggagaagcccttcgggagggacctgcaga
gctctgaccggctgtcc
RPS27A (GenBank Accession No. NM_002954 (SEQ. ID NO: 17))
RPS27A Forward primer (SEQ. ID NO: 18): CTGGCAAGCAGCTGGAAGAT
RPS27A Reverse primer (SEQ. ID NO: 19): TTTCTTAGCACCACCACGAAGTC
RPS27A-amplicon (SEQ. ID NO: 20): CTGGCAAGCAGCTGGAAGATGGACGTA
CTTTGTCTGACTACAATATTCAAAAGGAGTCTA
CTCTTCATCTTGTGTTGAGACTTCGTGGTGGTGCTAAGAAA

The sequences of the housekeeping genes measured in all the examples in the lung panel were as follows:

Ubiquitin (GenBank Accession No. BC000449 (SEQ. ID NO: 29))
Ubiquitin Forward primer (SEQ. ID NO: 30): ATTTGGGTCGCGGTTCTTG
Ubiquitin Reverse primer (SEQ. ID NO: 31): TGCCTTGACATTCTCGATGGT
Ubiquitin-amplicon (SEQ. ID NO: 32) ATTTGGGTCGCGGTTCTTGTTTGTG
GATCGCTGTGATCGTCACTTGACAATGCAGATCTTC
GTGAAGACTCTGACTGGTAAGACCATCACCCTCGAGG
TTGAGCCCAGTGACACCATCGAGAATGTCAAGGCA
SDHA (GenBank Accession No. NM_004168 (SEQ. ID NO: 33))
SDHA Forward primer (SEQ. ID NO: 34): TGGGAACAAGAGGGCATCTG
SDHA Reverse primer (SEQ. ID NO: 35): CCACCACTGCATCAAATTCATG
SDHA-amplicon (SEQ. ID NO: 36): TGGGAACAAGAGGGCATCTGCTAAA
GTTTCAGATTCCATTTCTGCTCAGTATCCAGTAGT
GGATCATGAATTTGATGCAGTGGTGG
PBGD (GenBank Accession No. BC019323 (SEQ. ID NO: 1)),
PBGD Forward primer (SEQ. ID NO: 2): TGAGAGTGATTCGCGTGGG
PBGD Reverse primer (SEQ. ID NO: 3): CCAGGGTACGAGGCTTTCAAT
PBGD-amplicon (SEQ. ID NO: 4): TGAGAGTGATTCGCGTGGGTACCCGC
AAGAGCCAGCTTGCTCGCATACAGACGGACAGT
GTGGTGGCAACATTGAAAGCCTCGTACCCTGG
HPRT1 (GenBank Accession No. NM_000194 (SEQ. ID NO: 5)),
HPRT1 Forward primer (SEQ. ID NO: 6): TGACACTGGCAAAACAATGCA
HPRT1 Reverse primer (SEQ. ID NO: 7): GGTCCTTTTCACCAGCAAGCT
HPRT1-amplicon (SEQ. ID NO: 8): TGACACTGGCAAAACAATGCAGACT
TTGCTTTCCTTGGTCAGGCAGTATAATCCAAAGAT
GGTCAAGGTCGCAAGCTTGCTGGTGAAAAGGACC

The sequences of the housekeeping genes measured in all the examples on breast cancer panel were as follows:

G6PD (GenBank Accession No. NM_000402 (SEQ. ID NO: 13))
G6PD Forward primer (SEQ. ID NO: 14): gaggccgtcaccaagaacat
G6PD Reverse primer (SEQ. ID NO: 15): ggacagccggtcagagctc
G6PD-amplicon (SEQ. ID NO: 16): gaggccgtcaccaagaacattcacgagtcctgca
tgagccagataggctggaaccgcatcatcgtggagaagcccttcgggagggacctgcaga
gctctgaccggctgtcc
SDHA (GenBank Accession No. NM_004168 (SEQ. ID NO: 33))
SDHA Forward primer (SEQ. ID NO: 34): TGGGAACAAGAGGGCATCTG
SDHA Reverse primer (SEQ. ID NO: 35): CCACCACTGCATCAAATTCATG
SDHA-amplicon (SEQ. ID NO: 36): TGGGAACAAGAGGGCATCTGCTAAA
GTTTCAGATTCCATTTCTGCTCAGTATCCAGTAGT
GGATCATGAATTTGATGCAGTGGTGG
PBGD (GenBank Accession No. BC019323 (SEQ. ID NO: 1)),
PBGD Forward primer (SEQ. ID NO: 2): TGAGAGTGATTCGCGTGGG
PBGD Reverse primer (SEQ. ID NO: 3): CCAGGGTACGAGGCTTTCAAT
PBGD-amplicon (SEQ. ID NO: 4): TGAGAGTGATTCGCGTGGGTACCCG
CAAGAGCCAGCTTGCTCGCATACAGACGGACAGT
GTGGTGGCAACATTGAAAGCCTCGTACCCTGG
HPRT1 (GenBank Accession No. NM_000194 (SEQ. ID NO: 5)),
HPRT1 Forward primer (SEQ. ID NO: 6): TGACACTGGCAAAACAATGCA
HPRT1 Reverse primer (SEQ. ID NO: 7): GGTCCTTTTCACCAGCAAGCT
HPRT1-amplicon (SEQ. ID NO: 8): TGACACTGGCAAAACAATGCAGACTT
TGCTTTCCTTGGTCAGGCAGTATAATCCAAAGAT
GGTCAAGGTCGCAAGCTTGCTGGTGAAAAGGACC

The sequences of the housekeeping genes measured in all the examples on normal tissue samples panel were as follows:

RPL19 (GenBank Accession No. NM_000981 (SEQ. ID NO: 21))
RPL19Forward primer (SEQ. ID NO: 22): TGGCAAGAAGAAGGTCTGGTTAG
RPL19Reverse primer (SEQ. ID NO: 23): TGATCAGCCCATCTTTGATGAG
RPL19-amplicon (SEQ. ID NO: 24): TGGCAAGAAGAAGGTCTGGTTAGAC
CCCAATGAGACCAATGAAATCGCCAATGCCAACT
CCCGTCAGCAGATCCGGAAGCTCATCAAAGATGGGCTGATCA
TATA box (GenBank Accession No. NM_003194 (SEQ. ID NO: 25)),
TATA box Forward primer (SEQ. ID NO: 26): CGGTTTGCTGCGGTAATCAT
TATA box Reverse primer (SEQ. ID NO: 27): TTTCTTGCTGCCAGTCTGGAC
TATA box-amplicon (SEQ. ID NO: 28): CGGTTTGCTGCGGTAATCATGAGGAT
AAGAGAGCCACGAACCACGGCACTGATTTTCAGTTCTGGGAAAATGGTGTG
CACAGGAGCCAAGAGTGAAGAACAGTCCAGACTGGCAGCAAGAAA
Ubiquitin (GenBank Accession No. BC000449 (SEQ. ID NO: 29))
Ubiquitin Forward primer (SEQ. ID NO: 30): ATTTGGGTCGCGGTTCTTG
Ubiquitin Reverse primer (SEQ. ID NO: 31): TGCCTTGACATTCTCGATGGT
Ubiquitin-amplicon (SEQ. ID NO: 32) ATTTGGGTCGCGGTTCTTGTTTGTGGATC
GCTGTGATCGTCACTTGACAATGCAGATCTTCGTGAAGACTCTGACTGGTAA
GACCATCACCCTCGAGGTTGAGCCCAGTGACACCATCGAGAATGTCAAGGCA
SDHA (GenBank Accession No. NM_004168 (SEQ. ID NO: 33))
SDHA Forward primer (SEQ. ID NO: 34): TGGGAACAAGAGGGCATCTG
SDHA Reverse primer (SEQ. ID NO: 35): CCACCACTGCATCAAATTCATG
SDHA-amplicon (SEQ. ID NO: 36): TGGGAACAAGAGGGCATCTGCTAAAGTTTC
AGATTCCATTTCTGCTCAGTATCCAGTAGTGGATCATGAATTTGATGCAGTGGTGG

### Actual Marker Examples

The following examples relate to specific actual marker examples. It should be noted that Table numbering is restarted within each example related to a particular Cluster, as indicated by the titles below.

### Description for Cluster N43992

Cluster N43992 features 3 transcript(s) and 14 segment(s) of interest, the names for which are given in Tables 2 and 3, respectively. The selected encoded protein variants are given in table 4.

TABLE 2
Transcripts of interest
Transcript Name

N43992_T1 (SEQ. ID NO: 37)
N43992_T4 (SEQ. ID NO: 38)
N43992_T9 (SEQ. ID NO: 39)

TABLE 3
Segments of interest
Segment Name

N43992_N0 (SEQ. ID NO: 40)
N43992_N5 (SEQ. ID NO: 41)
N43992_N9 (SEQ. ID NO: 42)
N43992_N12 (SEQ. ID NO: 43)
N43992_N14 (SEQ. ID NO: 44)
N43992_N15 (SEQ. ID NO: 45)
N43992_N17 (SEQ. ID NO: 46)
N43992_N22 (SEQ. ID NO: 47)
N43992_N1 (SEQ. ID NO: 48)
N43992_N3 (SEQ. ID NO: 49)
N43992_N4 (SEQ. ID NO: 50)
N43992_N7 (SEQ. ID NO: 51)
N43992_N10 (SEQ. ID NO: 52)
N43992_N20 (SEQ. ID NO: 53)

TABLE 4
Proteins of interest

| Protein Name | Corresponding Transcript(s) |
|---|---|
| N43992_P13 (SEQ. ID NO: 57) | N43992_T1 (SEQ. ID NO: 37) |
| N43992_P14 (SEQ. ID NO: 58) | N43992_T4 (SEQ. ID NO: 38) |
| N43992_P16 (SEQ. ID NO: 59) | N43992_T9 (SEQ. ID NO: 39) |

These sequences are variants of the known protein Delta-like protein 3 precursor (SEQ. ID NO: 54, SwissProt accession identifier DLL3_HUMAN); known also according to the synonym Drosophila Delta homolog 3, referred to herein as the previously known protein. The nucleic acid sequence of the known protein Delta-like protein 3 precursor is given in SEQ. ID NOs: 54 -56.

Protein Delta-like protein 3 precursor is known or believed to have the following function(s): inhibits primary neurogenesis; may be required to divert neurons along a specific differentiation pathway; play a role in the formation of somite boundaries during segmentation of the paraxial mesoderm (by similarity). Known polymorphisms for this sequence are as shown in Table 5.

TABLE 5
Amino acid mutations for Known Protein

| SNP position(s) on | |
|---|---|
| amino acid sequence | Comment |
| 218 | L -> P (in dbSNP:1110627)./FTId = VAR_016776 |
| 385 | G -> D (in SCDO1)./FTId = VAR_009952 |

Protein Delta-like protein 3 precursor (SEQ. ID NO: 54) localization is believed to be Type I membrane protein.

The following GO Annotation(s) apply to the previously known protein. The following annotation(s) were found: cell fate determination; embryonic development (sensu Mammalia); neurogenesis; Notch signaling pathWay; skeletal development, which are annotation(s) related to Biological Process; Notch binding, which are annotation(s) related to Molecular Function; and integral to membrane, which are annotation(s) related to Cellular Component.

The GO assignment relies on information from one or more of the SwissProt/TremBI Protein knowledgebase, available from <http://www.expasy.ch/sprot/>; or Locuslink, available from <http://www.ncbi.nlm.nih.gov/projects/LocusLink/>.

The present invention provides a number of different novel amino acid and nucleic acid sequences of known DLL3 protein, which may optionally be used as diagnostic markers, preferably as serum markers.

The variant N43992_P16 (SEQ. ID NO: 59) was previously disclosed by the inventors in published PCT application no WO2005/071058, hereby incorporated by reference as if fully set forth herein, but have now been shown to have novel and surprising diagnostic uses as described herein for other variants of cluster N43992.

According to the present invention, the known (wild type) DLL3 protein is used as novel diagnostic marker. According to the present invention, the wild type DLL3 protein diagnostic marker is optionally used with in vivo imaging technologies, including but not limited to magnetic resonance imaging, computed tomography scanning, PET, SPECT and the like. Optionally, according to the present invention, the wild type DLL3 protein diagnostic marker is used as IHC marker.

According to optional but preferred embodiments of the present invention, variants of this cluster according to the present invention (amino acid and/or nucleic acid sequences of N43992) may optionally have one or more of the utilities based on the finding that mutations in DLL3 cause axial skeletal defects in spondylocostal dysostosis (10742114). It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

Other non-limiting exemplary utilities for N43992 variants, according to the present invention are described in greater detail below and also with regard to the previous section on clinical utility.

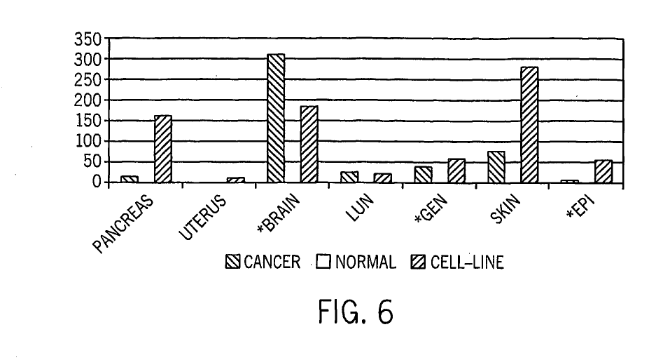

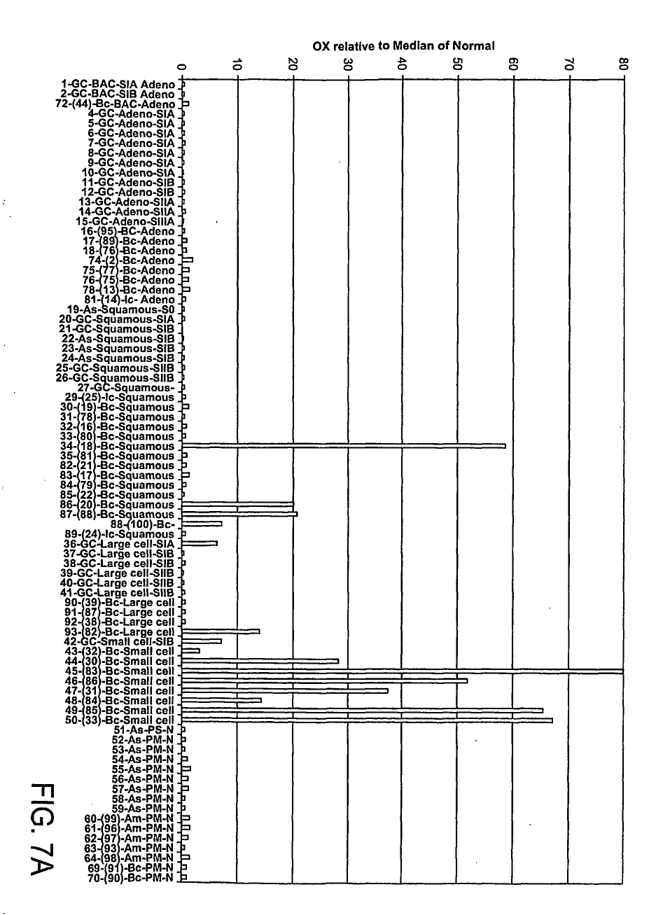

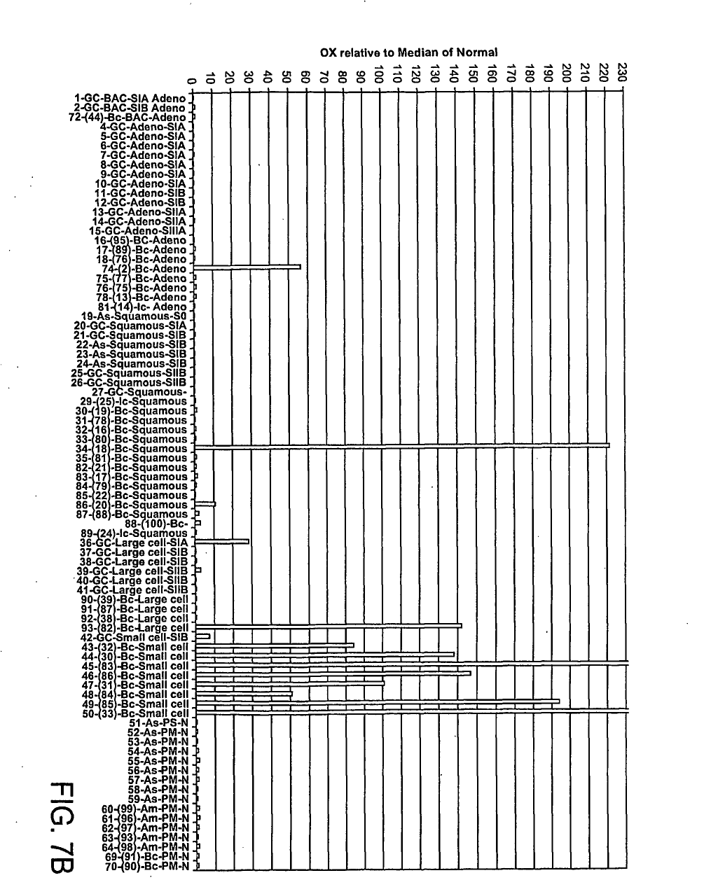

Cluster N43992 can be used as a diagnostic marker according to overexpression of transcripts of this cluster in cancer. Expression of such transcripts in normal tissues is also given according to the previously described methods. The term “number” in the left hand column of the table and the numbers on the y-axis of FIG. 6 refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 6 and Table 6. This cluster is overexpressed (at least at a minimum level) in the following pathological conditions: brain malignant tumors, a mixture of malignant tumors from different tissues and epithelial malignant tumors.

TABLE 6
Normal tissue distribution

| Name of Tissue | Number |
|---|---|
| pancreas | 0 |
| uterus | 0 |
| brain | 3 |
| lung | 0 |
| general | 1 |
| skin | 0 |
| epithelial | 0 |

TABLE 7
P values and ratios for expression in cancerous tissue

| Name of Tissue | P1 | P2 | SP1 | R3 | SP2 | R4 |
|---|---|---|---|---|---|---|
| pancreas | 3.1e−01 | 1.6e−01 | 4.2e−01 | 2.4 | 1.1e−02 | 3.7 |
| uterus | N/A | 3.7e−01 | N/A | N/A | 8.0e−01 | 1.3 |
| brain | 1.5e−05 | 6.0e−06 | 1.0e−19 | 35.3 | 1.1e−21 | 30.3 |
| lung | 4.7e−01 | 3.7e−01 | 4.1e−01 | 3.7 | 2.3e−01 | 3.4 |
| general | 2.4e−05 | 2.9e−05 | 4.2e−17 | 16.6 | 2.2e−28 | 18.4 |
| skin | 3.3e−01 | 2.4e−01 | 1.5e−01 | 6.8 | 1.9e−07 | 2.7 |
| epithelial | 2.3e−01 | 1.0e−01 | 7.8e−02 | 3.7 | 3.8e−08 | 6.0 |

As noted above, cluster N43992 features 3 transcript(s), which were listed in Table 2 above. These transcript(s) encode for protein(s) which are variant(s) of protein Delta-like protein 3 precursor (SEQ. ID NO: 54). A description of each variant protein according to the present invention is now provided.

Variant protein N43992_P13 (SEQ. ID NO: 57) according to the present invention is encoded by transcript N43992_T1 (SEQ. ID NO:37).

### 1. Comparison Report Between N43992_P13 (SEQ. ID NO: 57) and DLL3_HUMAN (SEQ. ID NO: 54):

A. An isolated chimeric polypeptide encoding for N43992_P13 (SEQ. ID NO: 57), comprising a first amino acid sequence being at least 90% homologous to MVSPRMSGLLSQTVILALIFLPQTRPAGVFELQIHSFGPGPGP corresponding to amino acids 1-43 of DLL3_HUMAN (SEQ. ID NO: 54), which also corresponds to amino acids 1-43 of N43992_P13 (SEQ. ID NO: 57), and a second amino acid sequence being at least 70%, optionally at least 80%, preferably at least 85%, more preferably at least 90% and most preferably at least 95% homologous to a polypeptide having the sequence APLPPLLQSLPEAWALRGGRRVPVRPGRGAECARTGLHRAARSARA (SEQ. ID NO: 326) corresponding to amino acids 44-89 of N43992_P13 (SEQ. ID NO: 57), wherein said first amino acid sequence and second amino acid sequence are contiguous and in a sequential order.

B. An isolated polypeptide encoding for an edge portion of N43992_P13 (SEQ. ID NO: 57), comprising an amino acid sequence being at least 70%, optionally at least about 80%, preferably at least about 85%, more preferably at least about 90% and most preferably at least about 95% homologous to the sequence APLPPLLQSLPEAWALRGGRRVPVRPGRGAECARTGLHRAARSARA (SEQ. ID NO: 326) of N43992_P13 (SEQ. ID NO: 57).

The localization of the variant protein was determined according to results from a number of different software programs and analyses, including analyses from SignalP and other specialized programs. The variant protein is secreted.

Variant protein N43992_P13 (SEQ. ID NO: 57) also has the following non-silent SNPs (Single Nucleotide Polymorphisms) as listed in Table 8, (given according to their position(s) on the amino acid sequence, with the alternative amino acid(s) listed; the last column indicates whether the SNP is known or not; the presence of known SNPs in variant protein N43992_P13 (SEQ. ID NO: 57) sequence provides support for the deduced sequence of this variant protein according to the present invention).

TABLE 8
Amino acid mutations

| SNP position(s) on amino acid | |
|---|---|
| sequence | Alternative amino acid(s) |
| 44 | A -> |

Variant protein N43992_P13 (SEQ. ID NO: 57) is encoded by the following transcript(s): N43992_T1 (SEQ. ID NO:37), for which the coding portion starts at position 71 and ends at position 337. The transcript also has the following SNPs as listed in Table 9 (given according to their position on the nucleotide sequence, with the alternative nucleic acid listed; the last column indicates whether the SNP is known or not; the presence of known SNPs in variant protein N43992_P13 (SEQ. ID NO: 57) sequence provides support for the deduced sequence of this variant protein according to the present invention).

TABLE 9
Nucleic acid SNPs

| SNP position(s) on nucleotide | |
|---|---|
| sequence | Alternative nucleic acid(s) |
| 201 | C -> |
| 509 | C -> |
| 556 | T -> G |
| 587 | C -> G |
| 626 | C -> |
| 694 | T -> C |
| 953 | C -> T |
| 1070 | C -> T |
| 1588 | G -> T |
| 1626 | C -> T |
| 1844 | T -> |
| 1890 | G -> T |

### 1. Comparison Report Between N43992_P14 (SEQ. ID NO: 58) and DLL3_HUMAN (SEQ. ID NO: 54):

A. An isolated chimeric polypeptide encoding for N43992_P14 (SEQ. ID NO: 58), comprising a first amino acid sequence being at least 90% homologous to MVSPRMSGLLSQTVILALIFLPQ corresponding to amino acids 1-23 of DLL3_HUMAN (SEQ. ID NO: 54), which also corresponds to amino acids 1-23 of N43992_P14 (SEQ. ID NO: 58), and a second amino acid sequence being at least 70%, optionally at least 80%, preferably at least 85%, more preferably at least 90% and most preferably at least 95% homologous to a polypeptide having the sequence VRARHGPLASSSCRSTLSGRVQALGPRGPPAAPGSPAASSSESA (SEQ. ID NO: 327) corresponding to amino acids 24-67 of N43992_P14 (SEQ. ID NO: 58), wherein said first amino acid sequence and second amino acid sequence are contiguous and in a sequential order.

B. An isolated polypeptide encoding for an edge portion of N43992_P14 (SEQ. ID NO: 58), comprising an amino acid sequence being at least 70%, optionally at least about 80%, preferably at least about 85%, more preferably at least about 90% and most preferably at least about 95% homologous to the sequence VRARHGPLASSSCRSTLSGRVQALGPRGPPAAPGSPAASSSESA (SEQ. ID NO: 327) of N43992_P14 (SEQ. ID NO: 58).

The localization of the variant protein was determined according to results from a number of different software programs and analyses, including analyses from SignalP and other specialized programs. The variant protein is secreted.

Variant protein N43992_P14 (SEQ. ID NO: 58) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein N43992_P14 (SEQ. ID NO: 58) sequence provides support for the deduced sequence of this variant protein according to the present invention).

TABLE 10
Amino acid mutations

| SNP position(s) on amino acid | |
|---|---|
| sequence | Alternative amino acid(s) |
| 57 | G -> |

Variant protein N43992_P14 (SEQ. ID NO: 58) is encoded by the following transcript(s): N43992_T4 (SEQ. ID NO:38), for which the coding portion starts at position 71 and ends at position 271. The transcript also has the following SNPs as listed in Table 11 (given according to their position on the nucleotide sequence, with the alternative nucleic acid listed; the last column indicates whether the SNP is known or not; the presence of known SNPs in variant protein N43992_P14 (SEQ. ID NO: 58) sequence provides support for the deduced sequence of this variant protein according to the present invention).

TABLE 11
Nucleic acid SNPs

| SNP position(s) on nucleotide | |
|---|---|
| sequence | Alternative nucleic acid(s) |
| 241 | C -> |
| 549 | C -> |
| 596 | T -> G |
| 627 | C -> G |
| 666 | C -> |
| 734 | T -> C |
| 993 | C -> T |
| 1110 | C -> T |
| 1628 | G -> T |
| 1666 | C -> T |
| 1884 | T -> |
| 1930 | G -> T |

Variant protein N43992_P16 (SEQ. ID NO: 59) according to the present invention is encoded by transcript(s) N43992_T9 (SEQ. ID NO:39). One or more alignments to one or more previously published Delta-like protein 3 precursor (SEQ. ID NO: 54) protein sequences are given in the alignment table on the attached CD-ROM. A brief description of the relationship of the variant protein according to the present invention to each such aligned protein is as follows:

### 1. Comparison Report Between N43992_P16 (SEQ. ID NO: 59) and DLL3_HUMAN (SEQ. ID NO: 54):

A. An isolated chimeric polypeptide encoding for N43992_P16 (SEQ. ID NO: 59), comprising a first amino acid sequence being at least 90% homologous to MVSPRMSGLLSQTVlLALIFLPQTRPAGVFELQINSFGPGPGPGAPRSPCSARLPCRLFFRVCLK PGLSEEAAESPCALGAALSARGPVYTEQPGAPAPDLPLPDGLLQVPFRDAWPGITSFECETWRE ELGDQIGGPAWSLLARVAGRRRLAAGGPWARDIQRAGAWELRFSYRARCEPPAVGTACTRL CRPRSAPSRCGPGLRPCAPLEDECEAPLVCRAGCSPEHGFCEQPGECRCLEGWTGPLCTVPVS TSSCLSPRGPSSATTGCLVPGPGPCDGNPCANGGSCSETPRSFECTCPRGFYGLRCEVSGVTCA DGPCFNGGLCVGGADPDSAYICHCPPGFQGSNCEKRVDRCSLQPCRNG corresponding to amino acids 1-365 of DLL3_HUMAN (SEQ. ID NO: 54), which also corresponds to amino acids 1-365 of N43992_P16 (SEQ. ID NO: 59), and a second amino acid sequence being at least 70%, optionally at least 80%, preferably at least 85%, more preferably at least 90% and most preferably at least 95% homologous to a polypeptide having the sequence EAWRPERRGMGWGSWMAQTVQGWNPGFDSSNPRAWGPDLPPASL (SEQ. ID NO: 328) corresponding to amino acids 366-409 of N43992_P16 (SEQ. ID NO: 59), wherein said first amino acid sequence and second amino acid sequence are contiguous and in a sequential order.

B. An isolated polypeptide encoding for an edge portion of N43992_P16 (SEQ. ID NO: 59), comprising an amino acid sequence being at least 70%, optionally at least about 80%, preferably at least about 85%, more preferably at least about 90% and most preferably at least about 95% homologous to the sequence EAWRPERRGMGWGSWMAQTVQGWNPGFDSSNPRAWGPDLPPASL (SEQ. ID NO: 328) of N43992_P16 (SEQ. ID NO: 59).

### 3. Comparison Report Between N43992_P16 (SEQ. ID NO: 59) and Q8NBS4_HUMAN (SEQ. ID NO: 55)

A. An isolated chimeric polypeptide encoding for N43992_P16 (SEQ. ID NO: 59), comprising a first amino acid sequence being at least 90% homologous to MVSPRMSGLLSQTVILALIFLPQTRPAGVFELQIHSFGPGPGPGAPRSPCSARLPCRLFFRVCLK PGLSEEAAESPCALGAALSARGPVYTEQPGAPAPDLPLPDGLLQVPFRDAWPGTFSFIIETWRE ELGDQIGGPAWSLLARVAGRRRLAAGGPWARDIQRAGAWELR corresponding to amino acids 1-171 of Q8NBS4_HUMAN (SEQ. ID NO: 55), which also corresponds to amino acids 1-171 of N43992_P16 (SEQ. ID NO: 59), a bridging amino acid F corresponding to amino acid 172 of N43992P16 (SEQ. ID NO: 59), a second amino acid sequence being at least 90% homologous to SYRARCEPPAVGTACTRLCRPRSAPSRCGPGLRPCAPLEDECEAP corresponding to amino acids 173-217 of Q8NBS4_HUMAN (SEQ. ID NO: 55), which also corresponds to amino acids 173-217 of N43992_P16 (SEQ. ID NO: 59), a bridging amino acid L corresponding to amino acid 218 of N43992_P16 (SEQ. ID NO: 59), a third amino acid sequence being at least 90% homologous to VCRAGCSPEHGFCEQPGECRCLEGWTGPLCTVPVSTSSCLSPRGPSSATTGCLVPGPGPCDGN PCANGGSCSETPRSFECTCPRGFYGLRCEVSGVTCA corresponding to amino acids 219-317 of Q8NBS4_HUMAN (SEQ. ID NO: 55), which also corresponds to amino acids 219-317 of N43992_P16 (SEQ. ID NO: 59), a bridging amino acid D corresponding to amino acid 318 of N43992_P16 (SEQ. ID NO: 59), a fourth amino acid sequence being at least 90% homologous to GPCFNGGLCVGGADPDSAYICHCPPGFQGSNCEKRVDRCSLQPCRNG corresponding to amino acids 319-365 of Q8NBS4HUMAN (SEQ. ID NO: 55), which also corresponds to amino acids 319-365 of N43992_P16 (SEQ. ID NO: 59), and a fifth amino acid sequence being at least 70%, optionally at least 80%, preferably at least 85%, more preferably at least 90% and most preferably at least 95% homologous to a polypeptide having the sequence EAWRPERRGMGWGSWMAQTVQGWNPGFDSSNPRAWGPDLPPASL (SEQ. ID NO: 328) corresponding to amino acids 366-409 of N43992_P16 (SEQ. ID NO: 59), wherein said first amino acid sequence, bridging amino acid, second amino acid sequence, bridging amino acid, third amino acid sequence, bridging amino acid, fourth amino acid sequence and fifth amino acid sequence are contiguous and in a sequential order.

B. An isolated polypeptide encoding for an edge portion of N43992_P16 (SEQ. ID NO: 59), comprising an amino acid sequence being at least 70%, optionally at least about 80%, preferably at least about 85%, more preferably at least about 90% and most preferably at least about 95% homologous to the sequence EAWRPERRGMGWGSWMAQTVQGWNPGFDSSNPRAWGPDLPPASL (SEQ. ID NO: 328) of N43992_P16 (SEQ. ID NO: 59).

The localization of the variant protein was determined according to results from a number of different software programs and analyses, including analyses from SignalP and other specialized programs. The variant protein is secreted.

Variant protein N43992_P16 (SEQ. ID NO: 59) also has the following non-silent SNPs (Single Nucleotide Polymorphisms) as listed in Table 12, (given according to their position(s) on the amino acid sequence, with the alternative amino acid(s) listed; the last column indicates whether the SNP is known or not; the presence of known SNPs in variant protein N43992_P16 (SEQ. ID NO: 59) sequence provides support for the deduced sequence of this variant protein according to the present invention).

TABLE 12
Amino acid mutations

| SNP position(s) on amino acid | |
|---|---|
| sequence | Alternative amino acid(s) |
| 54 | L -> |
| 156 | G -> |
| 172 | F -> C |
| 195 | S -> |
| 218 | L -> P |

The variant protein has the following domains, as determined by using InterPro. The domains are described in Table 13:

TABLE 13
InterPro domain(s)

| Domain description | Analysis type | Position(s) on protein |
|---|---|---|
| Type II EGF-like signature | FPrintScan | 274-285, 286-293, |
| | | 335-345, 346-352 |
| EGF-like | HMMPfam | 278-309, 316-350 |
| EGF-like calcium-binding | HMMSmart | 279-310, 316-351 |
| Type I EGF | HMMSmart | 213-249, 277-310, 315-351 |
| EGF-like, subtype 2 | ProfileScan | 278-309, 316-350 |
| EGF-like | ScanRegExp | 237-248, 298-309, 339-350 |
| Myb, DNA-binding | ScanRegExp | 140-162 |
| EGF-like | ScanRegExp | 237-248, 298-309, 339-350 |

Variant protein N43992_P16 (SEQ. ID NO: 59) is encoded by the following transcript(s): N43992_T9 (SEQ. ID NO:39), for which the coding portion starts at position 71 and ends at position 1297. The transcript also has the following SNPs as listed in Table 14 (given according to their position on the nucleotide sequence, with the alternative nucleic acid listed; the last column indicates whether the SNP is known or not; the presence of known SNPs in variant protein N43992_P16 (SEQ. ID NO: 59) sequence provides support for the deduced sequence of this variant protein according to the present invention).

TABLE 14
Nucleic acid SNPs

| SNP position(s) on nucleotide | |
|---|---|
| sequence | Alternative nucleic acid(s) |
| 230 | C -> |
| 538 | C -> |
| 585 | T -> G |
| 616 | C -> G |
| 655 | C -> |
| 723 | T -> C |
| 982 | C -> T |
| 1099 | C -> T |

As noted above, cluster N43992 features 14 segment(s), which were listed in Table 3 above and for which the sequence(s) are given. These segment(s) are portions of nucleic acid sequence(s) which are described herein separately because they are of particular interest. A description of several segments according to the present invention is now provided.

Segment cluster N43992_N0 (SEQ. ID NO: 40) according to the present invention is supported by 15 libraries. The number of libraries was determined as previously described. This segment can be found in the following transcript(s): N43992_T1 (SEQ. ID NO:37), N43992_T4 (SEQ. ID NO:38) and N43992_T9 (SEQ. ID NO:39). Table 15 below describes the starting and ending position of this segment on each transcript.

TABLE 15
Segment location on transcripts

| | Segment | Segment |
|---|---|---|
| Transcript name | starting position | ending position |
| N43992_T1 (SEQ. ID NO: 37) | 1 | 139 |
| N43992_T4 (SEQ. ID NO: 38) | 1 | 139 |
| N43992_T9 (SEQ. ID NO: 39) | 1 | 139 |

Segment cluster N43992_N5 (SEQ. ID NO: 41) according to the present invention is supported by 20 libraries. The number of libraries was determined as previously described. This segment can be found in the following transcript(s): N43992_T1 (SEQ. ID NO:37), N43992_T4 (SEQ. ID NO:38) and N43992_T9 (SEQ. ID NO:39). Table 16 below describes the starting and ending position of this segment on each transcript.

TABLE 16
Segment location on transcripts

| | Segment | Segment |
|---|---|---|
| Transcript name | starting position | ending position |
| N43992_T1 (SEQ. ID NO: 37) | 198 | 392 |
| N43992_T4 (SEQ. ID NO: 38) | 238 | 432 |
| N43992_T9 (SEQ. ID NO: 39) | 227 | 421 |

Segment cluster N43992_N12 (SEQ. ID NO: 43) according to the present invention is supported by 22 libraries. The number of libraries was determined as previously described. This segment can be found in the following transcript(s): N43992_T1 (SEQ. ID NO:37), N43992_T4 (SEQ. ID NO:38) and N43992_T9 (SEQ. ID NO:39). Table 17 below describes the starting and ending position of this segment on each transcript.

TABLE 17
Segment location on transcripts

| | Segment | Segment |
|---|---|---|
| Transcript name | starting position | ending position |
| N43992_T1 (SEQ. ID NO: 37) | 694 | 911 |
| N43992_T4 (SEQ. ID NO: 38) | 734 | 951 |
| N43992_T9 (SEQ. ID NO: 39) | 723 | 940 |

Segment cluster N43992_N15 (SEQ. ID NO: 45) according to the present invention is supported by 2 libraries. The number of libraries was determined as previously described. This segment can be found in the following transcript(s): N43992_T9 (SEQ. ID NO:39). Table 18 below describes the starting and ending position of this segment on each transcript.

TABLE 18
Segment location on transcripts

| | Segment | Segment |
|---|---|---|
| Transcript name | starting position | ending position |
| N43992_T9 (SEQ. ID NO: 39) | 1164 | 1866 |

According to an optional embodiment of the present invention, short segments related to the above cluster are also provided. These segments are up to about 120 by in length, and so are included in a separate description.

Segment cluster N43992_N1 (SEQ. ID NO: 48) according to the present invention is supported by 1 libraries. The number of libraries was determined as previously described. This segment can be found in the following transcript(s): N43992_T4 (SEQ. ID NO:38). Table 19 below describes the starting and ending position of this segment on each transcript.

TABLE 19
Segment location on transcripts

| | Segment | Segment |
|---|---|---|
| Transcript name | starting position | ending position |
| N43992_T4 (SEQ. ID NO: 38) | 140 | 150 |

Segment cluster N43992_N3 (SEQ. ID NO: 49) according to the present invention is supported by 18 libraries. The number of libraries was determined as previously described. This segment can be found in the following transcript(s): N43992_T1 (SEQ. ID NO:37), N43992_T4 (SEQ. ID NO:38) and N43992_T9 (SEQ. ID NO:39). Table 20 below describes the starting and ending position of this segment on each transcript.

TABLE 20
Segment location on transcripts

| | Segment | Segment |
|---|---|---|
| Transcript name | starting position | ending position |
| N43992_T1 (SEQ. ID NO: 37) | 140 | 197 |
| N43992_T4 (SEQ. ID NO: 38) | 151 | 208 |
| N43992_T9 (SEQ. ID NO: 39) | 140 | 197 |

Expression of Homo Sapiens Delta-Like 3 (Drosophila) (DLL3) N43992 Transcripts Which are Detectable by Taqman Probes as Depicted in Sequence Names N43992-T4 (SEQ. ID NO: 64) and N43992-T4II (SEQ. ID NO: 68) in Normal and Cancerous Lung Tissues

Expression of Homo sapiens delta-like 3 (Drosophila) (DLL3) transcripts detectable by or according to junction1-3 was measured by real time PCR with MGB-Taqman probes and primers as follows:
1. Probe: N43992T4 (SEQ. ID NO: 64) and primers Fwd: N43992seg0-1F-TaqDan (SEQ. ID NO: 65) and Rev: N43992seg3R-taq (SEQ. ID NO: 66)
2. Probe: N43992T4II (SEQ. ID NO: 68) and primers Fwd: N43992seg0E-Taq (SEQ. ID NO: 69) and Rev: N43992seg3R-taq (SEQ. ID NO: 66)

In the experiment carried out with probe N43992T4 (SEQ. ID NO: 64) samples 1, 2, 4-20, 22-27, 29-33, 35, 37-41, 51-64, 69, 70, 72, 74-76, 78, 81-85, 89 and 90-92 were undetected. In the experiment carried out with probe N43992T4II (SEQ. ID NO: 68) samples 1, 4-27, 29-33, 35, 37, 38, 40, 41, 51-64, 69, 70, 72, 75, 76, 78, 81-85, 87, 89 and 90-92 were undetected. Undetected samples were assigned a value of 41 and calculated accordingly. In parallel the expression of four housekeeping genes—HPRT1 (GenBank Accession No. NM_{—}000194 (SEQ. ID NO: 5); amplicon—HPRT1-amplicon (SEQ. ID NO: 8)), PBGD (GenBank Accession No. BC019323 (SEQ. ID NO: 1); amplicon—PBGD-amplicon (SEQ. ID NO: 4)), SDHA (GenBank Accession No. NM_{—}004168 (SEQ. ID NO: 33); amplicon—SDHA-amplicon (SEQ. ID NO:36)) and Ubiquitin (GenBank Accession No. BC000449 (SEQ. ID NO: 29); amplicon—Ubiquitin-amplicon (SEQ. ID NO: 32)) was measured by real time PCR with SYBR green detection. For each RT sample, the expression of the above amplicon was normalized to the normalization factor calculated from the expression of these house keeping genes as described in “Real-Time RT-PCR analysis using TaqMan® probes” in the “materials and methods” section. The normalized quantity of each RT sample was then divided by the median of the quantities of the normal samples (sample numbers 51-64, 69, 70 Table 1_{—}5 above), to obtain a value of fold up-regulation for each sample relative to median of the normal samples. In the experiment done with probe N43992T4 (SEQ. ID NO: 64) samples 1, 2, 4-20, 22-27, 29-33, 35, 37-41, 51-64, 69, 70, 72, 74-76, 78, 81-85, 89 and 90-92 were undetected.

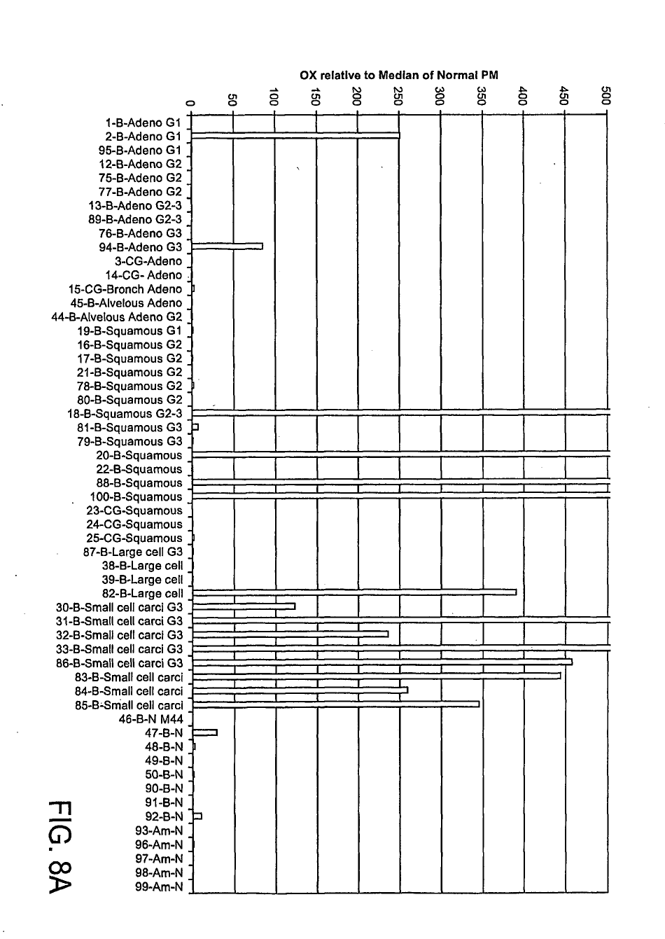

FIGS. 7A and 7B are histograms showing over expression of the above-indicated Homo sapiens delta-like 3 (Drosophila) (DLL3) transcripts in cancerous Lung samples relative to the normal samples using the 2 different Taqman probes. FIG. 7A—results using N43992T4 (SEQ. ID NO: 64) probe, FIG. 7B—results using N43992T4II (SEQ. ID NO: 68) probe.

As is evident from FIGS. 7A and 7B the expression of Homo sapiens delta-like 3 (Drosophila) (DLL3) transcripts detectable by the above primers and probes in small cell carcinoma samples was significantly higher than in the non-cancerous samples (sample numbers 51-64, 69, 70 Table 1_{—}5 above). Notably an over-expression of at least 5 fold was found in 8 out of 9 small cells carcinoma samples and 6 out of 57 non-small cell carcinoma samples (in 4 out of 23 squamous cell carcinoma samples and 2 out of 10 large cell carcinoma samples) when experiment was carried out with N43992T4 (SEQ. ID NO: 64) probe and over-expression of at least 5 fold was found in 9 out of 9 small cell carcinoma samples and 5 out of 57 non-small cell carcinoma samples (1 out of 24 adenocarcinoma samples, 2 out of 23 squamous cell carcinoma samples and 2 out of 10 large cell carcinoma samples) when experiment was carried out with N43992T4II (SEQ. ID NO: 68) probe.

Statistical analysis was applied to verify the significance of these results, as described below.

The P value for the difference in the expression levels of Homo sapiens delta-like 3 (Drosophila) (DLL3) transcripts detectable probes N43992T4 (SEQ. ID NO: 64) and N43992T4II (SEQ. ID NO: 68) in Lung small cell carcinoma samples versus the normal tissue samples was determined by T test as 1.76e-03 and 2.06e-03, respectively.

Threshold of 5 fold over expression of Homo sapiens delta-like 3 (Drosophila) (DLL3) transcripts detectable by probes N43992T4 (SEQ. ID NO: 64) and N43992T4II (SEQ. ID NO: 68) was found to differentiate between small cell carcinoma and normal samples with P value of 8.32e-06 and 4.89e-07, respectively, as checked by exact Fisher test.

The above values demonstrate statistical significance of the results.

Primer pairs and probes are also optionally and preferably encompassed within the present invention; for example, for the above experiment, the following primer pairs and probes were used as a non-limiting illustrative example only of a suitable primer pairs:
1. Probe: N43992T4 (SEQ. ID NO: 64) and primers Fwd: N43992seg0-1F-TaqDan (SEQ. ID NO: 65) and Rev: N43992seg3R-taq (SEQ. ID NO: 66)
2. Probe: N43992T4II (SEQ. ID NO: 68) and primers Fwd: N43992seg0E-Taq (SEQ. ID NO: 69) and Rev: N43992seg3R-taq (SEQ. ID NO: 66)

The present invention also preferably encompasses any amplicon obtained through the use of any suitable primer pair; for example, for the above experiment, the following amplicon was obtained as a non-limiting illustrative example only of a suitable amplicon: N43992_junc1-3 (SEQ. ID NO: 67), and N43992_junc1-3II (SEQ. ID NO: 70).

1. TaqMan″ MGB probe: Name: N43992T4 (SEQ. ID NO: 64) - FAM-AGCCAGACACGGCC-BQ
Forward Primer: N43992seg0-1F-TaqDan (SEQ. ID NO: 65) - TCATTTTCCTCCCCCAGGTC
Reverse Primer: N43992seg3R-taq (SEQ. ID NO: 66) - GTGGATCTGCAGCTCGAAGAC
Amplicon N43992_junc1-3 (SEQ. ID NO: 67): TCATTTTCCTCCCCCAGGTCAGAGCCAGA
CACGGCCCGCTGGCGTCTTCGAGCTGCAGATCCAC
2. TaqMan″ MGB probe: Name: N43992T4II (SEQ. ID NO: 68) - FAM-TCAGAGCCAGACACGG-
BQ
Forward Primer: N43992seg0F-Taq (SEQ. ID NO: 69) - TCCTCTCCCAGACTGTGATCCT
Reverse Primer: N43992seg3R-taq (SEQ. ID NO: 66) - GTGGATCTGCAGCTCGAAGAC
Amplicon N43992_junc1-3II (SEQ. ID NO: 70): TCCTCTCCCAGACTGTGATCCTAGCGCTC
ATTTTCCTCCCCCAGGTCAGAGCCAGACACGGCCCGCTGGCGTCTTCGAGCTGCAGATCCAC

Expression of Homo Sapiens Delta-Like 3 (Drosophila) (DLL3) N43992 Transcripts Which are Detectable by Amplicon as Depicted in Sequence name N43992_seg12WTF2R2 (SEQ. ID NO: 62) in Normal and Cancerous Lung Tissues

Expression of Homo sapiens delta-like 3 (Drosophila) (DLL3) transcripts detectable by or according to seg12WTF2R2-N43992_seg12WTF2R2 (SEQ. ID NO: 62) amplicon and primers N43992_seg12WTF2 (SEQ. ID NO: 60) and N43992_seg12WTR2 (SEQ. ID NO: 61), including but not limited to the known DLL3 transcript SEQ. ID NOs: 74, 75, was measured by real time PCR. In parallel the expression of four housekeeping genes—HPRT1 (GenBank Accession No. NM_{—}000194 (SEQ. ID NO: 5); amplicon—HPRT1-amplicon (SEQ. ID NO: 8), PBGD (GenBank Accession No. BC019323 (SEQ. ID NO: 1); amplicon—PBGD-amplicon (SEQ. ID NO: 4), SDHA (GenBank Accession No. NM_{—}004168 (SEQ. ID NO: 33); amplicon—SDHA-amplicon (SEQ. ID NO:36) and Ubiquitin (GenBank Accession No. BC000449 (SEQ. ID NO: 29); amplicon—Ubiquitin-amplicon (SEQ. ID NO: 32) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 47, 48, 49, 50, 90, 91, 92, 93, 96, 97 and 98, Table 1_{—}2 above), to obtain a value of fold up-regulation for each sample relative to median of the normal PM samples.

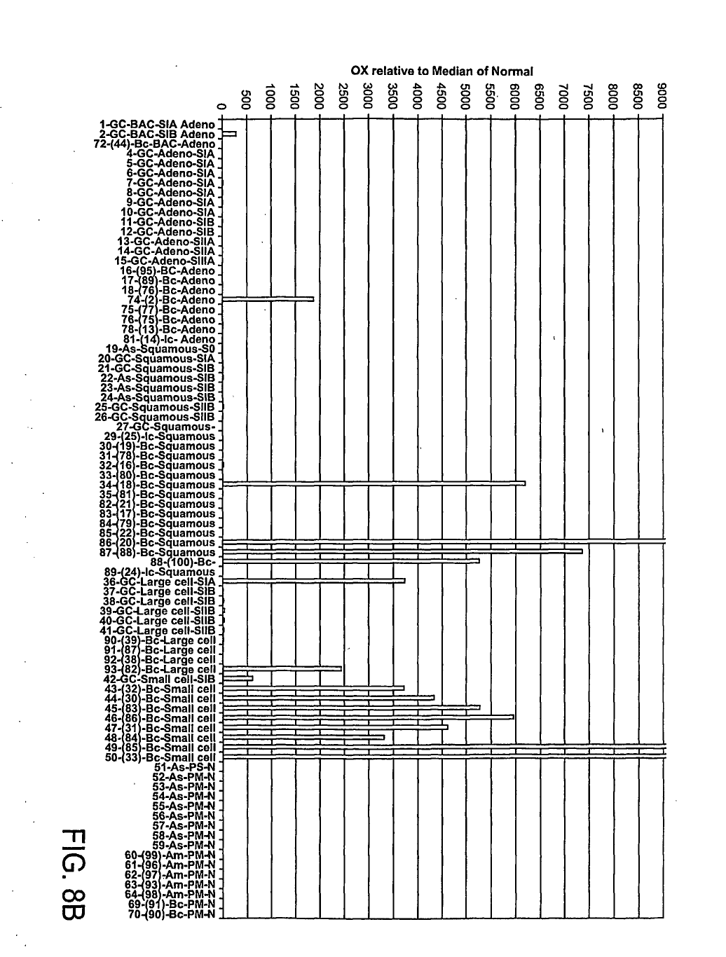

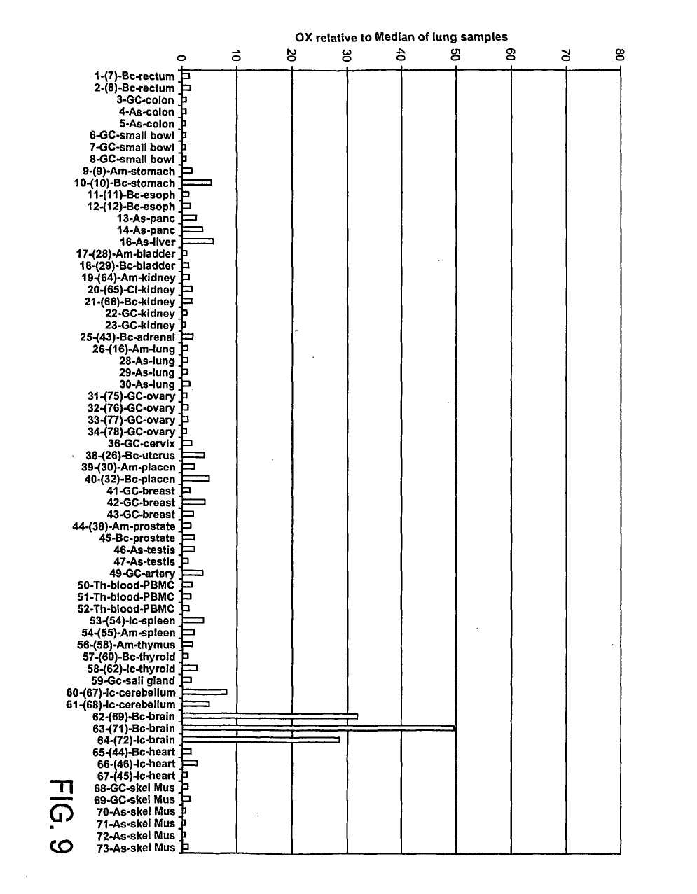

FIG. 8A is a histogram showing over expression of the above-indicated Homo sapiens delta-like 3 (Drosophila) (DLL3) transcripts in cancerous Lung samples relative to the normal samples.

As is evident from FIG. 8A, the expression of Homo sapiens delta-like 3 (Drosophila) (DLL3) transcripts detectable by the above amplicon in small cell carcinoma samples was significantly higher than in the non-cancerous samples (sample numbers 47, 48, 49, 50, 90, 91, 92, 93, 96, 97 and 98, Table 1_{—}2 above) and was higher in a few non-small cell carcinoma samples than in the non-cancerous samples. Notably an over-expression of at least 5 fold was found in 8 out of 8 small cell carcinoma samples, and in 8 out of 27 non-small cell carcinoma samples, specifically in 5 out of 13 squamous cell carcinoma samples.

Statistical analysis was applied to verify the significance of these results, as described below.

The P value for the difference in the expression levels of Homo sapiens delta-like 3 (Drosophila) (DLL3) transcripts detectable by the above amplicon in Lung small cell carcinoma samples versus the normal tissue samples was determined by T test as 1.21e-03. The P value for the difference in the expression levels of Homo sapiens delta-like 3 (Drosophila) (DLL3) transcripts detectable by the above amplicon in Lung non-small cell carcinoma samples versus the normal tissue samples was determined by T test as 1.76e-02. The P value for the difference in the expression levels of Homo sapiens delta-like 3 (Drosophila) (DLL3) transcripts detectable by the above amplicon in Lung squamous cell carcinoma samples versus the normal tissue samples was determined by T test as 4.58e-02.

Threshold of 5 fold over expression was found to differentiate between small cell carcinoma and normal samples with P value of 5.95e-04 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: N43992_seg12WTF2 (SEQ. ID NO: 60) forward primer; and N43992_seg12WTR2 (SEQ. ID NO: 61) reverse primer.

The present invention also preferably encompasses any amplicon obtained through the use of any suitable primer pair; for example, for the above experiment, the following amplicon was obtained as a non-limiting illustrative example only of a suitable amplicon: N43992_seg12WTF2R2 (SEQ. ID NO: 62).

Forward Primer (N43992_seg12WTF2
(SEQ. ID NO: 60))-TGTGAACAGCCCGGTGAA
Reverse Primer (N43992_seg12WTR2
(SEQ. ID NO: 61))-GACAAGGCATCCGGTGGTAG
Amplicon (N43992_seg12WTF2R2
(SEQ. ID NO: 62))
TGTGAACAGCCCGGTGAATGCCGATGCCTAGAGGGCTGGACTGGACCC
CTCTGCAC GGTCCCTGTCTCCACCAGCAGCTGCCTCAGCCCCAGGGGC
CCGTCCTCTGCTACCACCGGATGCCTTGTC

### Expression of Homo Sapiens Delta-Like 3 (Drosophila) (DLL3) N43992 Transcripts Which are Detectable by Taqman Probe as Depicted in Sequence Names N43992T3 (SEQ. ID NO: 71) in Normal and Cancerous Lung Tissues

Expression of Homo sapiens delta-like 3 (Drosophila) (DLL3) transcripts detectable by or according to junction0-3 was measured by real time PCR with MGB-Taqman probe N43992T3 (SEQ. ID NO: 71) and primers N43992seg0E-Taq (SEQ. ID NO: 69) and N43992seg3WT R-Taq (SEQ. ID NO: 72). Samples 4, 6, 14, 16-18, 24, 51-57, 60-62, 64, 69, 70, 72, 75, 78, 81-83 and 89 were undetected. These samples were assigned a value of 41 and calculated accordingly. In parallel the expression of four housekeeping genes—HPRT1 (GenBank Accession No. NM_{—}000194 (SEQ. ID NO: 5); amplicon—FPRT1-amplicon (SEQ. ID NO: 8)), PBGD (GenBank Accession No. BC019323 (SEQ. ID NO: 1); amplicon—PBGD-amplicon (SEQ. ID NO: 4)), SDHA (GenBank Accession No. NM_{—}004168 (SEQ. ID NO: 33); amplicon—SDHA-amplicon (SEQ. ID NO:36)) and Ubiquitin (GenBank Accession No. BC000449 (SEQ. ID NO: 29); amplicon—Ubiquitin-amplicon (SEQ. ID NO: 32)) was measured by real time PCR with SYBR green detection. For each RT sample, the expression of the above amplicon was normalized to the normalization factor calculated from the expression of these house keeping genes as described in “Real-Time RT-PCR analysis using TaqMan® probes” in the “materials and methods” section. The normalized quantity of each RT sample was then divided by the median of the quantities of the normal samples (sample numbers 51-64, 69, 70 Table 1_{—}5 above), to obtain a value of fold up-regulation for each sample relative to median of the normal samples.

FIG. 8B is a histogram showing over expression of the above-indicated Homo sapiens delta-like 3 (Drosophila) (DLL3) transcripts in cancerous Lung samples relative to the normal samples.

As is evident from FIG. 8B, the expression of Homo sapiens delta-like 3 (Drosophila) (DLL3) transcripts detectable by the above primers and probe in small cell carcinoma samples was significantly higher than in the non-cancerous samples (sample numbers 51-64, 69, 70 Table 1_{—}5 above). Notably an over-expression of at least 250 fold was found in 9 out of 9 small cell carcinoma samples and in 8 out 57 non-small cell carcinoma samples (2 out of 24 adenocarcinoma samples, 4 out of 23 squamous cell carcinoma samples and 2 out of 10 large cell carcinoma samples).

Statistical analysis was applied to verify the significance of these results, as described below.

The P value for the difference in the expression levels of Homo sapiens delta-like 3 (Drosophila) (DLL3) transcripts detectable probe N43992T3 (SEQ. ID NO: 71) in Lung small cell carcinoma samples and non-small cell carcinoma versus the normal tissue samples was determined by T test as 3.41e-04 and 9.52e-03, respectively.

Threshold of 250 fold over expression of Homo sapiens delta-like 3 (Drosophila) (DLL3) transcripts detectable by probe N43992T3 (SEQ. ID NO: 71) was found to differentiate between small cell carcinoma and normal samples with P value of 4.89e-07, as checked by exact Fisher test.

The above values demonstrate statistical significance of the results.

Primer pairs and probes are also optionally and preferably encompassed within the present invention; for example, for the above experiment, the following primer pairs and probes were used as a non-limiting illustrative example only of a suitable primer pairs: Probe: N43992T3 (SEQ. ID NO: 71) and primers Fwd: N43992seg0E-Taq (SEQ. ID NO: 69) and Rev: N43992seg3WT_R-Taq (SEQ. ID NO: 72)

The present invention also preferably encompasses any amplicon obtained through the use of any suitable primer pair; for example, for the above experiment, the following amplicon was obtained as a non-limiting illustrative example only of a suitable amplicon: N43992 junc0-3 (SEQ. ID NO: 73)

TaqMan″ MGB probe: Name: N43992T3
(SEQ. ID NO: 71)-VIC-CCTCCCCCAGACACG-BQ
Forward Primer: N43992seg0F-Taq
(SEQ. ID NO: 69)-TCCTCTCCCAGACTGTGATCCT
Reverse Primer: N43992seg3WT_R-Taq
(SEQ. ID NO: 72)-ACCCGGCCCGAAAGAGT
Amplicon N43992_junc0-3
(SEQ. ID NO: 73): TCCTCTCCCAGACTGTGATCCTAGCGCTCAT
TTTCCTCCCCCAGACACGGCCCGCTGGCGTCTTCGAGCTGCA
GATCCACTCTTTCGGGCCGGGT

### Expression of Homo Sapiens Delta-Like 3 (Drosophila) (DLL3) N43992 Transcripts Which are Detectable by Taqman Probe as Depicted in Sequence Names N43992-T4 (SEQ. ID NO: 64) in Different Normal Tissues

Expression of Homo sapiens delta-like 3 (Drosophila) (DLL3) transcripts detectable by or according to junction1-3 was measured by real time PCR with MGB-Taqman probe N43992T4 (SEQ. ID NO: 64) and primers: Fwd: N43992seg0-1F-TaqDan (SEQ. ID NO: 65) and Rev: N43992seg3R-taq (SEQ. ID NO: 66). Samples 1-14, 16-23, 25-27, 28-34, 36, 38-45, 49-54, 56-59 and 65-73 were undetected. These samples were assigned a value of 41 and calculated accordingly. In parallel the expression of three housekeeping genes—SDHA (GenBank Accession No. NM_{—}004168 (SEQ. ID NO: 33); amplicon—SDHA-amplicon (SEQ. ID NO:36)), Ubiquitin (GenBank Accession No. BC000449 (SEQ. ID NO: 29); amplicon—Ubiquitin-amplicon (SEQ. ID NO: 32)), and TATA box (GenBank Accession No. NM_{—}003194 (SEQ. ID NO: 25); TATA amplicon (SEQ. ID NO: 28)) was measured by real time PCR with SYBR green detection. For each RT sample, the expression of the above amplicon was normalized to the normalization factor calculated from the expression of these house keeping genes as described in “Real-Time RT-PCR analysis using TaqMan® probes” in the “materials and methods” section. The normalized quantity of each RT sample was then divided by the median of the quantities of the lung samples (sample numbers 28, 29 and 30, Table 1_{—}7 above), to obtain a value of relative expression of each sample relative to median of the Lung samples.

TaqMan″ MGB probe: Name: N43992T4II
(SEQ. ID NO: 68): FAM-TCAGAGCCAGACACGG-BQ
Forward Primer: N43992seg0F-Taq (SEQ. ID NO: 69)
TCCTCTCCCAGACTGTGATCCT
Reverse Primer: N43992seg3R-taq (SEQ. ID NO: 66)
GTGGATCTGCAGCTCGAAGAC
Amplicon (SEQ. ID NO: 70): TCCTCTCCCAGACTGTGATCCT
AGCGCTCATTTTCCTCCCCCAGGTCAGAGCCAGACACGGCCCGCTGGCGT
CTTCGAGCTGCAGATCCAC

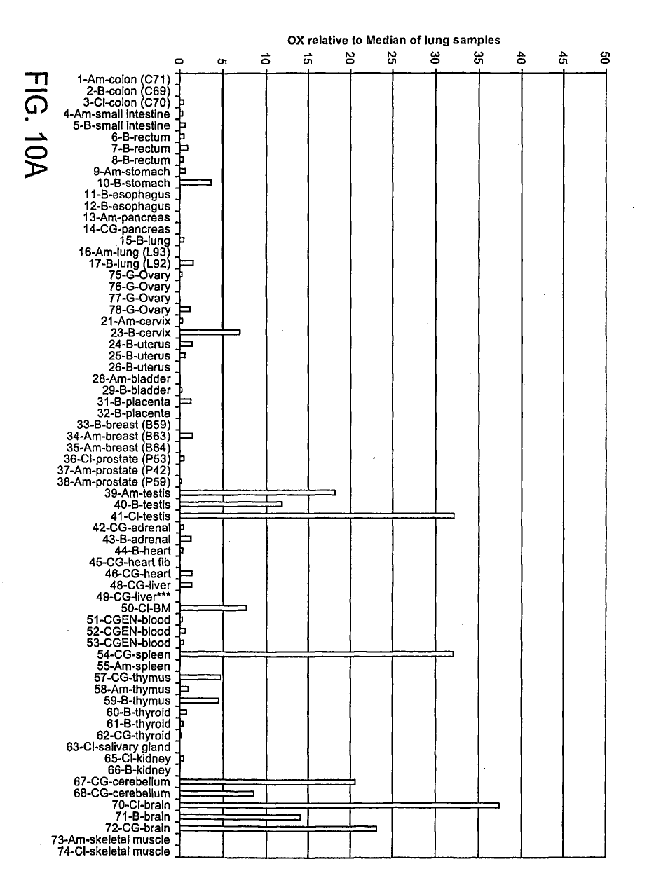

FIG. 9 is a histogram showing expression of Homo sapiens delta-like 3 (Drosophila) (DLL3) N43992 transcripts which are detectable by Taqman probe as depicted in sequence names N43992-T4 (SEQ. ID NO: 64) in different normal tissues.

### Expression of Homo Sapiens Delta-Like 3 (Drosophila) (DLL3) N43992 Transcripts Which are Detectable by Amplicon as Depicted in Sequence Name N43992_seg12WTF2R2 (SEQ. ID NO: 62) in Different Normal Tissues

Expression of Homo sapiens delta-like 3 (Drosophila) (DLL3) transcripts detectable by or according to seg12WTF2R2-N43992_seg12WTF2R2 (SEQ. ID NO: 62) amplicon and primers N43992_seg12WTF2 (SEQ. ID NO: 60) and N43992_seg12WTR2 (SEQ. ID NO: 61), including but not limited to the known DLL3 transcript SEQ. ID NOs: 74, 75, was measured by real time PCR. In parallel the expression of four housekeeping genes—SDHA (GenBank Accession No. NM_{—}004168 (SEQ. ID NO: 33); amplicon—SDHA-amplicon (SEQ. ID NO:36), Ubiquitin (GenBank Accession No. BC000449 (SEQ. ID NO: 29); amplicon—Ubiquitin-amplicon. (SEQ. ID NO: 32), RPL19 (GenBank Accession No. NM_{—}000981 (SEQ. ID NO: 21); RPL19 amplicon (SEQ. ID NO: 24) and TATA box (GenBank Accession No. NM_{—}003194 (SEQ. ID NO: 25); TATA amplicon (SEQ. ID NO: 28) was measured similarly. For each RT sample, the expression of the above amplicon was normalized to the geometric mean of the quantities of the housekeeping genes. The normalized quantity of each RT sample was then divided by the median of the quantities of the lung samples (sample numbers 15 and 17, Table 1_{—}6 above), to obtain a value of relative expression of each sample relative to median of the lung samples.

Forward Primer (N43992_seg12WTF2
(SEQ. ID NO: 60): TGTGAACAGCCCGGTGAA
Reverse Primer (N43992_seg12WTR2
(SEQ. ID NO: 61): GACAAGGCATCCGGTGGTAG
Amplicon (N43992_seg12WTF2R2
(SEQ. ID NO: 62): TGTGAACAGCCCGGTGAATGCCGATGCCTAGA
GGGCTGGACTGGACCCCTCTGCACGGTCCCTGTCTCCACCAGC
AGCTGCCTCAGCCCCAGGGGCCCGTCCTCTGCTACCACCGG
ATGCCTTGTC

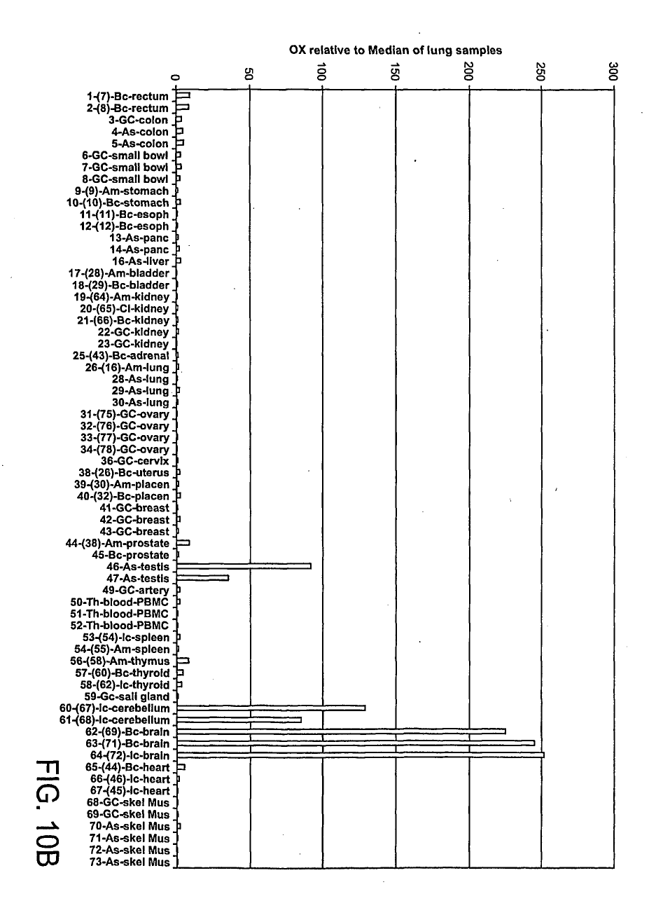

FIG. 10A is a histogram showing over expression of the Homo sapiens delta-like 3 (Drosophila) (DLL3) N43992 transcripts which are detectable by amplicon as depicted in sequence name N43992_seg12WTF2R2 (SEQ. ID NO: 62) in different normal tissues.

### Expression of Homo Sapiens Delta-Like 3 (Drosophila) (DLL3) N43992 Transcripts Which are Detectable by Taqman Probe as Depicted in Sequence Names N43992T3 (SEQ. ID NO: 71) in Different Normal Tissues.

Expression of Homo sapiens delta-like 3 (Drosophila) (DLL3) transcripts detectable by or according to junction0-3 was measured by real time PCR with MGB-Taqman probe N43992T3 (SEQ. ID NO: 71) and primers: N43992seg0E-Taq (SEQ. ID NO: 69) and N43992seg3WT_R-Taq (SEQ. ID NO: 72). Samples 9-14, 16-21, 23, 25, 28, 30-34, 36, 38-43, 45, 49, 51-54, 59, 66-69, 72 and 73 were undetected. These samples were assigned a value of 41 and calculated accordingly. In parallel the expression of three housekeeping genes—SDHA (GenBank Accession No. NM_{—}004168 (SEQ. ID NO: 33); amplicon—SDHA-amplicon (SEQ. ID NO:36)), Ubiquitin (GenBank Accession No. BC000449 (SEQ. ID NO: 29); amplicon—Ubiquitin-amplicon (SEQ. ID NO: 32)), and TATA box (GenBank Accession No. NM_{—}003194 (SEQ. ID NO: 25); TATA amplicon (SEQ. ID NO: 28)) was measured by real time PCR with SYBR green detection. For each RT sample, the expression of the above amplicon was normalized to the normalization factor calculated from the expression of these house keeping genes as described in “Real-Time RT-PCR analysis using TaqMan® probes” in the “materials and methods” section. The normalized quantity of each RT sample was then divided by the median of the quantities of the lung samples (sample numbers 28, 29 and 30, Table 1_{—}7 above), to obtain a value of relative expression of each sample relative to median of the Lung samples.

TaqMan″ MGB probe: Name: N43992T3 (SEQ. ID NO: 71)
VIC-CCTCCCCCAGACACG-BQ
Forward Primer: N43992seg0F-Taq
(SEQ. ID NO: 69)-TCCTCTCCCAGACTGTGATCCT
Reverse Primer: N43992seg3WT_R-Taq
(SEQ. ID NO: 72)-ACCCGGCCCGAAAGAGT
Amplicon (SEQ. ID NO: 73): TCCTCTCCCAGACTGTGATCCT
AGCGCTCATTTTCCTCCCCCAGACACGGCCCGCTGGCGT
CTTCGAGCTGCAGATCCACTCTTTCGGGCCGGGT

FIG. 10B is a histogram showing expression of Homo sapiens delta-like 3 (Drosophila) (DLL3) N43992 transcripts which are detectable by Taqman probe as depicted in sequence names N43992T3 (SEQ. ID NO: 71) in different normal tissues.

### Description for Cluster D12115

Cluster D12115 features 11 transcript(s) and 42 segment(s) of interest, the names for which are given in Tables 21 and 22, respectively. The selected protein variants are given in table 23.

TABLE 21
Transcripts of interest
Transcript Name

D12115_T3 (SEQ. ID NO: 78)
D12115_T5 (SEQ. ID NO: 79)
D12115_T9 (SEQ. ID NO: 80)
D12115_T12 (SEQ. ID NO: 81)
D12115_T13 (SEQ. ID NO: 82)
D12115_T14 (SEQ. ID NO: 83)
D12115_T19 (SEQ. ID NO: 84)
D12115_T22 (SEQ. ID NO: 85)
D12115_T27 (SEQ. ID NO: 86)
D12115_T33 (SEQ. ID NO: 87)
D12115_T36 (SEQ. ID NO: 88)

TABLE 22
Segments of interest
Segment Name

D12115_N0 (SEQ. ID NO: 89)
D12115_N2 (SEQ. ID NO: 90)
D12115_N4 (SEQ. ID NO: 91)
D12115_N5 (SEQ. ID NO: 92)
D12115_N6 (SEQ. ID NO: 93)
D12115_N7 (SEQ. ID NO: 94)
D12115_N26 (SEQ. ID NO: 95)
D12115_N27 (SEQ. ID NO: 96)
D12115_N34 (SEQ. ID NO: 97)
D12115_N41 (SEQ. ID NO: 98)
D12115_N53 (SEQ. ID NO: 99)
D12115_N3 (SEQ. ID NO: 100)
D12115_N11 (SEQ. ID NO: 101)
D12115_N12 (SEQ. ID NO: 102)
D12115_N14 (SEQ. ID NO: 103)
D12115_N15 (SEQ. ID NO: 104)
D12115_N16 (SEQ. ID NO: 105)
D12115_N17 (SEQ. ID NO: 106)
D12115_N20 (SEQ. ID NO: 107)
D12115_N21 (SEQ. ID NO: 108)
D12115_N22 (SEQ. ID NO: 109)
D12115_N23 (SEQ. ID NO: 110)
D12115_N24 (SEQ. ID NO: 111)
D12115_N28 (SEQ. ID NO: 112)
D12115_N29 (SEQ. ID NO: 113)
D12115_N31 (SEQ. ID NO: 114)
D12115_N32 (SEQ. ID NO: 115)
D12115_N33 (SEQ. ID NO: 116)
D12115_N36 (SEQ. ID NO: 117)
D12115_N38 (SEQ. ID NO: 118)
D12115_N39 (SEQ. ID NO: 119)
D12115_N43 (SEQ. ID NO: 120)
D12115_N45 (SEQ. ID NO: 121)
D12115_N46 (SEQ. ID NO: 122)
D12115_N47 (SEQ. ID NO: 123)
D12115_N48 (SEQ. ID NO: 124)
D12115_N49 (SEQ. ID NO: 125)
D12115_N50 (SEQ. ID NO: 126)
D12115_N52 (SEQ. ID NO: 127)
D12115_N54 (SEQ. ID NO: 128)
D12115_N55 (SEQ. ID NO: 129)
D12115_N56 (SEQ. ID NO: 130)

TABLE 23
Proteins of interest

| Protein Name | Corresponding Transcript(s) |
|---|---|
| D12115_P3 (SEQ. ID NO: 134) | D12115_T3 (SEQ. ID NO: 78) |
| D12115_P5 (SEQ. ID NO: 135) | D12115_T36 (SEQ. ID NO: 88); |
| | D12115_T5 (SEQ. ID NO: 79) |
| D12115_P12 (SEQ. ID NO: 136) | D12115_T12 (SEQ. ID NO: 81) |
| D12115_P13 (SEQ. ID NO: 137) | D12115_T13 (SEQ. ID NO: 82) |
| D12115_P15 (SEQ. ID NO: 138) | D12115_T19 (SEQ. ID NO: 84) |
| D12115_P16 (SEQ. ID NO: 139) | D12115_T22 (SEQ. ID NO: 85) |
| D12115_P20 (SEQ. ID NO: 140) | D12115_T27 (SEQ. ID NO: 86) |
| D12115_P32 (SEQ. ID NO: 141) | D12115_T33 (SEQ. ID NO: 87) |
| D12115_P34 (SEQ. ID NO: 142) | D12115_T14 (SEQ. ID NO: 83) |
| D12115_P35 (SEQ. ID NO: 143) | D12115_T9 (SEQ. ID NO: 80) |

These sequences are variants of the known protein Complement factor B precursor (SEQ. ID NO:131) (SwissProt accession identifier CFAB_HUMAN (SEQ. ID NO: 395); known also according to the synonyms EC 3.4.21.47; C3/C5 convertase; Properdin factor B; Glycine-rich beta glycoprotein; GBG; PBF2), referred to herein as the previously known protein.

The variant D12115_P3 (SEQ. ID NO:134) was previously disclosed by the inventors in published PCT application no WO2005/071058, and the variants D12115 P12 (SEQ. ID NO:136) and D12115_P16 (SEQ. ID NO:139) were previously disclosed by the inventors in published PCT applications no WO2005/071058 and WO2004/096979, hereby incorporated by reference as if fully set forth herein, but have now been shown to have novel and surprising diagnostic uses as described herein for other variants of cluster D12115.

Protein Complement factor B precursor (SEQ. ID NO:131) is known or believed to have the following function(s): Factor B which is part of the alternate pathway of the complement system is cleaved by factor D into 2 fragments: Ba and Bb. Bb, a serine protease, then combines with complement factor 3b to generate the C3 or C5 convertase. It has also been implicated in proliferation and differentiation of preactivated B lymphocytes, rapid spreading of peripheral blood monocytes, stimulation of lymphocyte blastogenesis and lysis of erythrocytes. Ba inhibits the proliferation of preactivated B lymphocytes. Known polymorphisms for this sequence are as shown in Table 24.

TABLE 24
Amino acid mutations for Known Protein

| SNP position(s) on | |
|---|---|
| amino acid sequence | Comment |
| 9 | L -> H. /FTId = VAR_016274 |
| 28 | W -> R (in allele S). /FTId = VAR_006492 |
| 28 | W -> Q (in allele FA; requires 2 nucleotide |
| | substitutions)./FTId = VAR_006493 |
| 32 | R -> Q (in allele S). /FTId = VAR_006494 |
| 32 | R -> W. /FTId = VAR_016275 |
| 252 | G -> S. /FTId = VAR_016276 |
| 565 | K -> E. /FTId = VAR_016277 |
| 651 | D -> E. /FTId = VAR_016278 |
| 736 | A -> S (in allele FA). /FTId = VAR_006495 |
| 297 | I -> T |
| 300 | V -> L |
| 328 | D -> V |
| 356-357 | KK -> EE |
| 537 | I -> T |
| 764 | L -> H |

Protein Complement factor B precursor (SEQ. ID NO:131) localization is believed to be Secreted.

The previously known protein also has the following indication(s) and/or potential therapeutic use(s): Infection, general; Traumatic shock. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Complement factor inhibitor. A therapeutic role for a protein represented by the cluster has been predicted. The cluster was assigned this field because there was information in the drug database or the public databases (e.g., described herein above) that this protein, or part thereof, is used or can be used for a potential therapeutic indication: Anti-inflammatory; Cardiovascular; Immunosuppressant; Neuroprotective; Recombinant, other; Septic shock treatment.

According to optional but preferred embodiments of the present invention, variants of this cluster according to the present invention (amino acid and/or nucleic acid sequences of D12115) may optionally have one or more of the following utilities, as described with regard to the Table below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

Table of Utilities for Variants of D12115, related to protein Complement factor B precursor (SEQ. ID NO:131):

TABLE 25

| Utility | Reason | Reference |
|---|---|---|
| Complement factor B | | 16164698 |
| allotypes in the | | |
| susceptibility and severity | | |
| of coeliac disease. | | |
| diagnosis of Alzheimer's | | 15920296 |
| disease (AD), Parkinson's | | |
| disease (PD), and multiple | | |
| sclerosis (MS) in the CSF, | | |
| by a verity of alternative | | |
| splice isoforms. | | |
| diagnosis of ischemic acute | | 15673300 |
| tubular necrosis, in serum | | |
| Complement factor B | | 12974797 |
| allotypes in the | | |
| susceptibility of Chagas | | |
| disease | | |

According to other optional embodiments of the present invention, variants of this cluster according to the present invention (amino acid and/or nucleic acid sequences of D12115) may optionally have one or more of the following utilities, some of which are related to utilities described above. It should be noted that these utilities are optionally and preferably suitable for human and non-human animals as subjects, except where otherwise noted.

The Table below describes diagnostic utilities for the cluster D12115 that were found through microarrays, including the statistical significance thereof and a reference. One or more D12115 variants according to the present invention may optionally have one or more of these utilities.

TABLE 26

| | |
|---|---|
| Differential diagnosis of in | GSE3325 |
| situ prostates cancer vs. | |
| metastasis (lower expression | |
| in metastasis). | |

According to further optional but preferred embodiments of the present invention, variants of this cluster according to the present invention (amino acid and/or nucleic acid sequences of D12115) may optionally have one or more of the following utilities, as described in greater detail below, which may also optionally be related to one or more of the above utilities. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

A non-limiting example of such a utility is the detection, diagnosis and/or determination of any condition that includes activation of the alternative complement pathway. For diagnostic utilities related to the activated form chain Bb, only variants T19 and T33,36 of the present invention are appropriate. The method comprises detecting a D12115 variant, for example a variant protein, protein fragment, peptide, polynucleotide, polynucleotide fragment and/or oligonucleotide as described herein, optionally and preferably in a serum sample. The expression levels of the D12115 variant as determined in a patient can be further compared to those in a normal individual.

For example, the known Protein Complement factor B has been shown to be useful for diagnosis of complement related immune disease, including but not limited to vasculitis, systemic lupus erythematosus, rheumatoid arthritis, myocardial infarction, ischemic/reperfusion injury, cerebrovascular accident, Alzheimer's disease, transplantation rejection (xeno and allo), all antibody-mediated skin diseases, all antibody-mediated organ-specific diseases (including Type I and Type 11 diabetes mellitus, thyroiditis, idiopathic thrombocytopenic purpura and hemolytic anemia, and neuropathies), multiple sclerosis, cardiopulmonary bypass injury, membranoproliferative glomerulonephritis, polyarteritis nodosa, Henoch Schonlein purpura, serum sickness, Goodpasture's disease, systemic necrotizing vasculitis, post streptococcal glomerulonephritis, idiopathic pulmonary fibrosis (usual interstitial pneumonitis) and membranous glomerulonephritis; breast, ovarian and prostate cancer; ovarian malignant hyperplasia.

Antibodies recognizing the known Protein Complement factor B are described as being useful for recognizing proteins in the serum of breast cancer patients that are differentially present with regard to PCT Application No. WO 02/088750, hereby incorporated by reference as if fully set forth herein.

Differential expression of the known Protein Complement factor B in prostate cancer tissues is described with regard to PCT Application No. WO 00/055174, hereby incorporated by reference as if fully set forth herein.

Differential expression of the known Protein Complement factor B in ovarian cancer tissues is described with regard to US Patent Application No. US2005095592, hereby incorporated by reference as if fully set forth herein.

Differential expression of the known Protein Complement factor B in ovarian malignant hyperplasia tissues is described with regard to U.S. Pat. No. 6,316,213, hereby incorporated by reference as if fully set forth herein.

Yet another non-limiting example of a utility is described in PCT Application No. WO 98/32390, hereby incorporated by reference as if fully set forth herein, for distinguishing bacterial meningitis from viral meningitis according to levels of complement B in cerebro-spinal fluid samples.

Yet another non-limiting example of a utility is described in PCT Application No. WO 04/055519, hereby incorporated by reference as if fully set forth herein, in which complement factor B was shown to be upregulated in pancreatic cancer tissue samples.

Yet another non-limiting example of a utility is described in U.S. Pat. No. 6,335,170, hereby incorporated by reference as if fully set forth herein, in which expression of complement factor B was shown to be upregulated in bladder cancer tissue samples.

The following GO Annotation(s) apply to the previously known protein. The following annotation(s) were found: complement activation, alternative pathway, which are annotation(s) related to Biological Process; and complement binding, which are annotation(s) related to Molecular Function.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Other non-limiting exemplary utilities for D12115 variants according to the present invention are described in greater detail below and also with regard to the previous section on clinical utility.

Cluster D12115 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

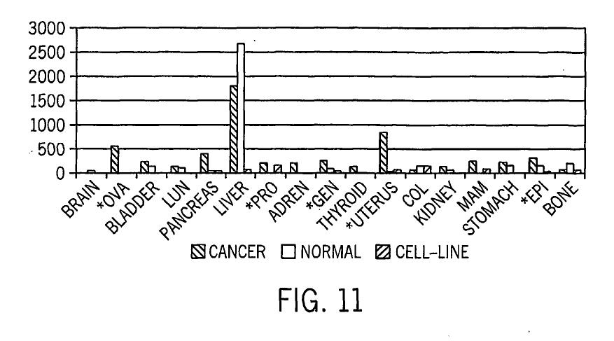

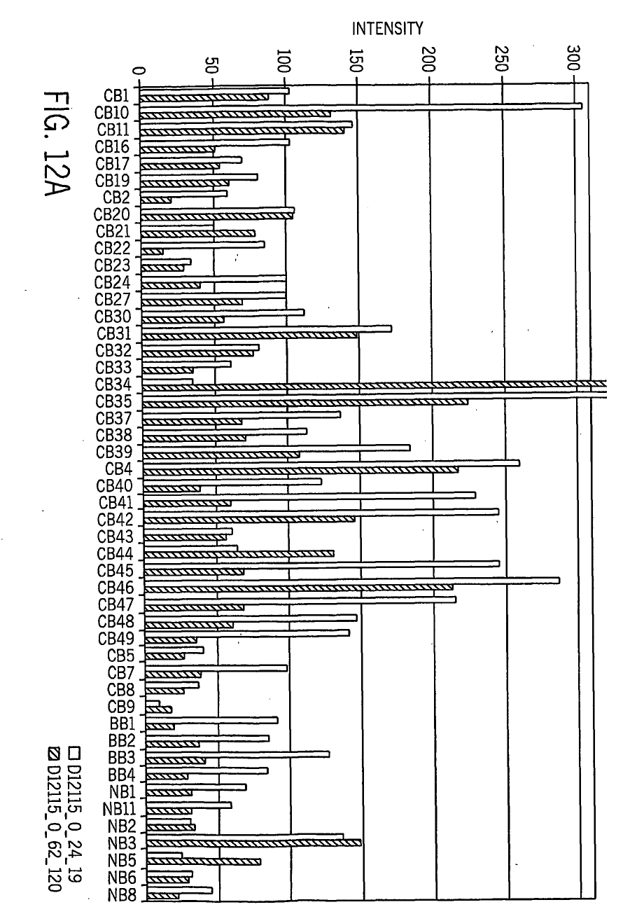

Overall, the following results were obtained as shown with regard to the histograms in FIG. 11 and Table 27. This cluster is overexpressed (at least at a minimum level) in the following pathological conditions: ovarian carcinoma, prostate cancer, a mixture of malignant tumors from different tissues, uterine malignancies and epithelial malignant tumors.

TABLE 27
Normal tissue distribution

| Name of Tissue | Number |
|---|---|
| brain | 31 |
| ovary | 0 |
| bladder | 123 |
| lung | 93 |
| pancreas | 42 |
| liver | 2675 |
| prostate | 1 |
| adrenal | 0 |
| general | 85 |
| Thyroid | 0 |
| uterus | 22 |
| colon | 141 |
| kidney | 65 |
| breast | 17 |
| stomach | 147 |
| epithelial | 159 |
| bone | 189 |

TABLE 28
P values and ratios for expression in cancerous tissue

| Name of Tissue | P1 | P2 | SP1 | R3 | SP2 | R4 |
|---|---|---|---|---|---|---|
| brain | 6.3e−01 | 8.0e−01 | 9.4e−01 | 0.6 | 1.0e+00 | 0.3 |
| ovary | 3.7e−03 | 7.0e−03 | 3.5e−06 | 13.0 | 1.6e−04 | 8.9 |
| bladder | 7.0e−01 | 7.8e−01 | 3.0e−01 | 1.0 | 6.0e−01 | 0.7 |
| lung | 1.1e−01 | 4.6e−01 | 2.9e−01 | 1.2 | 8.5e−01 | 0.6 |
| pancreas | 4.0e−01 | 3.8e−01 | 3.1e−07 | 2.9 | 3.3e−05 | 2.1 |
| liver | 4.9e−01 | 7.9e−01 | 9.2e−01 | 0.3 | 1.0e+00 | 0.1 |
| prostate | 1.1e−01 | 1.6e−01 | 4.9e−04 | 6.9 | 5.3e−04 | 7.1 |
| adrenal | 3.8e−01 | 4.3e−01 | 4.3e−02 | 3.4 | 8.0e−02 | 2.8 |
| general | 9.0e−06 | 8.7e−03 | 1.6e−25 | 2.2 | 7.8e−06 | 1.2 |
| Thyroid | 1.8e−01 | 1.8e−01 | 4.6e−01 | 2.0 | 4.6e−01 | 2.0 |
| uterus | 5.4e−04 | 5.1e−03 | 2.0e−12 | 6.0 | 4.3e−07 | 3.9 |
| colon | 5.9e−01 | 6.5e−01 | 9.4e−01 | 0.5 | 8.4e−01 | 0.5 |
| kidney | 2.3e−01 | 4.1e−01 | 2.4e−01 | 1.8 | 5.6e−01 | 1.2 |
| breast | 8.6e−02 | 8.9e−02 | 1.1e−02 | 4.0 | 4.3e−02 | 2.9 |
| stomach | 3.7e−01 | 8.4e−01 | 3.8e−01 | 0.8 | 9.6e−01 | 0.4 |
| epithelial | 2.8e−04 | 4.7e−02 | 2.4e−09 | 1.5 | 2.5e−01 | 0.9 |
| bone | 3.7e−01 | 3.2e−01 | 9.7e−01 | 0.4 | 9.9e−01 | 0.4 |

As noted above, cluster D12115 features 11 transcript(s), which were listed in Table 21 above. These transcript(s) encode for protein(s) which are variant(s) of protein Complement factor B precursor (SEQ. ID NO:131). A description of each variant protein according to the present invention is now provided.

Variant protein D12115_P3 (SEQ. ID NO:134) according to the present invention is encoded by transcript D12115_T3 (SEQ. ID NO:78). One or more alignments to one or more previously published Complement factor B precursor (SEQ. ID NO:131) protein sequences are given in the alignment table on the attached CD-ROM. A brief description of the relationship of the variant protein according to the present invention to each such aligned protein is as follows:

### 1. Comparison Report Between D12115_P3 (SEQ. ID NO:134) and CFAB_HUMAN (SEQ. ID NO: 395):

A. An isolated chimeric polypeptide encoding for D12115_P³ (SEQ. ID NO:134), comprising a first amino acid sequence being at least 90% homologous to MGSNLSPQLCLMPFILGLLSGGVTTTPWSLARPQGSCSLEGVEIKGGSFRLLQEGQALEYVCPS GFYPYPVQTRTCRSTGSWSTLKTQDQKTVRKAECRAIHCPRPHDFENGEYWPRSPYYNVSDE ISFHCYDGYTLRGSANRTCQVNGRWSGQTAICDNGAGYCSNPGIPIGTRKVGSQYRLEDSVT YHCSRGLTLRGSQRRTCQEGGSWSGTEPSCQDSFMYDTPQEVAEAFLSSLTETIEGVDAEDGH GPGEQQKRKIVLDPSGSMNIYLVLDGSDSIGASNFTGAKKCLVNLIEKVASYGVKPRYGLVTY ATYPKIWVKVSEADSSNADWVTKQLNEINYEDHICLKSGTNTKKALQAVYSMMSWPDDVPP EGWNRTRHVIILMTDGLHNNIGGDPITVIDEIRDLLYIGKDRKNPREDYLDVYVFGVGPLVNQ VNINALASKKDNEQHVFKVKDMENLEDVFYQMIDESQSLSLCGMVWEHRKGTDYFEKQPWQ AKISVIRPSKGHESCMGAVVSEYFVLTAAHCFTVDDKEHSIKVSVGGEICRDLEIEVVLFHPNY NINGKKEAGIPEFYDYDVALIKLKNKLKYGQTIRPICLPCTEGTTRALRLPPITTCQQQKEELL PAQDIKALFVSEEEKKLTRKEVYIKNGDKKGSCERDAQYAPGYDKVKDISEVVTPRFLCIGG VSPYADPNTCRGDSGGPLIVHKRSRFIQVGVISWGVVDVCKNQKR corresponding to amino acids 1-730 of CFAB_HUMAN (SEQ. ID NO: 395), which also corresponds to amino acids 1-730 of D12115_P3 (SEQ. ID NO:134), and a second amino acid sequence being at least 70%, optionally at least 80%, preferably at least 85%, more preferably at least 90% and most preferably at least 95% homologous to a polypeptide having the sequence AALAEGETPR (SEQ. ID NO: 329) corresponding to amino acids 731-740 of D12115_P3 (SEQ. ID NO:134), wherein said first amino acid sequence and second amino acid sequence are contiguous and in a sequential order.

B. An isolated polypeptide encoding for an edge portion of D12115_P3 (SEQ. ID NO:134), comprising an amino acid sequence being at least 70%, optionally at least about 80%, preferably at least about 85%, more preferably at least about 90% and most preferably at least about 95% homologous to the sequence AALAEGETPR (SEQ. ID NO: 329) of D12115_P3 (SEQ. ID NO:134).

### 2. Comparison Report Between D12115_P3 (SEQ. ID NO:134) and NP_{—}001701 (SEQ. ID NO:133):

A. An isolated chimeric polypeptide encoding for D12115_P3 (SEQ. ID NO:134), comprising a first amino acid sequence being at least 90% homologous to MGSNLSPQLCLMPFILGLLSGGVTTTPWSLA corresponding to amino acids 1-31 of NP_{—}001701 (SEQ. ID NO:133), which also corresponds to amino acids 1-31 of D12115_P3 (SEQ. ID NO:134), a bridging amino acid R corresponding to amino acid 32 of D12115_P3 (SEQ. ID NO:134), a second amino acid sequence being at least 90% homologous to PQGSCSLEGVEIKGGSFRLLQEGQALEYVCPSGFYPYPVQTRTCRSTGSWSTLKTQDQKTVRK AECRAEHCPRPHDFENGEYWPRSPYYNVSDEISFHCYDGYTLRGSANRTCQVNGRWSGQTAI CDNGAGYCSNPGIPIGTRKVGSQYRLEDSVTYHCSRGLTLRGSQRRTCQEGGSWSGTEPSCQ DSFMYDTPQEVAEAFLSSLTETIEGVDAEDGHGPGEQQICRICIVLDPSGSMNIYLVLDGSDSIG ASNFTGAKKCLVNLIEKVASYGVKPRYGLVTYATYPKIWVKVSEADSSNADWVTKQLNEIN YEDHICLKSGTNTICKALQAVYSMMSWPDDVPPEGWNRTRHVELMTDGIIINMGGDPITVLDE IRDLLYIGICDRICNPREDYLDVYVFGVGPLVNQVNBNIALASKKDNEQHVFKVKDMENLEDVF YQMEDESQSLSLCGMVWEHRKGTDYHKQPWQAKISVIRPSKGBESCMGAVVSEYFVLTAAH CFTVDDKEHSIKVSVGGEICRDLEIEVVLFHPNYNINGKICEAGEPEFYDYDVALIKLKNICLKYG QTIRPICLPCTEGTTRALRLPPTITCQQQKEELLPAQDLKALFVSEEEKKLTRKEVYIKNGDICK GSCERDAQYAPGYDKVICDISEVVTPRFLCTGGVSPYADPNTCRGDSGGPLIVHKRSRFIQVGV ISWGVVDVCKNQKR corresponding to amino acids 33-730 of NP_{—}001701 (SEQ. ID NO:133), which also corresponds to amino acids 33-730 of D12115_P3 (SEQ. ID NO:134), and a third amino acid sequence being at least 70%, optionally at least 80%, preferably at least 85%, more preferably at least 90% and most preferably at least 95% homologous to a polypeptide having the sequence AALAEGETPR (SEQ. ID NO: 329) corresponding to amino acids 731-740 of D12115_P3 (SEQ. ID NO:134), wherein said first amino acid sequence, bridging amino acid, second amino acid sequence and third amino acid sequence are contiguous and in a sequential order.

### 3. Comparison Report Between D12115_P3 (SEQ. ID NO:134) and P00751-2 (SEQ. ID NO:132):

A. An isolated chimeric polypeptide encoding for D12115_P3 (SEQ. ID NO:134), comprising a first amino acid sequence being at least 90% homologous to MGSNLSPQLCLMPFELGLLSGGVTTTPWSLARPQGSCSLEGVEIKGGSFRLLQEGQALEYVCPS GFYPYPVQTRTCRSTGSWSTLKTQDQKTVRKAECRAEHCPRPHDFENGEYWPRSPYYNVSDE ISFHCYDGYTLRGSANRTCQVNGRWSGQTAICDNGAGYCSNPGIPIGTRKVGSQYRLEDSVT YHCSRGLTLRGSQRRTCQEGGSWSGTEPSCQDSFMYDTPQEVAEAFLSSLTETIEGVDAEDGH GPGEQQKRKTVLDPSGSMNTYLVLDGSDSIGASNFTGAKKCINNUEKVASYGVKPRYGLVTY ATYPKIWVKVSEADSSNADWVTKQLNEINYEDHKLKSGTNTKKALQAVYSMMSWPDDVPP EGWNRTRHVIILMTDGLHNMGGDPITVIDEIRDLLYIGKDRKNPREDYLDVYVFGVGPLVNQ VNINALASICKDNEQHVFKVKDMENLEDVFYQMIDESQSLSLCGMVWEHRKGTDYHKQPWQ AKISVIRPSKGHESCMGAVVSEYFVLTAAHCFTVDDKEHSIKVSVG corresponding to amino acids 1-542 of P00751-2 (SEQ. ID NO:132), which also corresponds to amino acids 1-542 of D12115_P3 (SEQ. ID NO:134), and a second amino acid sequence being at least 70%, optionally at least 80%, preferably at least 85%, more preferably at least 90% and most preferably at least 95% homologous to a polypeptide having the sequence GEKRDLEIEVVLFHPNYNINGICKEAGIPEFYDYDVALIKLKNKLKYGQTIRPICLPCTEGTTRA LRLPPTTTCQQQKEELLPAQDIKALFVSEEEKKLTRKEVYIKNGDKKGSCERDAQYAPGYDK VKDISEVVTPRFLCTGGVSPYADPNTCRGDSGGPLIVIEKRSRFIQVGVISWGVVDVCKNQKRA ALAEGETPR (SEQ. ID NO: 330) corresponding to amino acids 543-740 of D12115_P3 (SEQ. ID NO:134), wherein said first amino acid sequence and second amino acid sequence are contiguous and in a sequential order.

B. An isolated polypeptide encoding for an edge portion of D12115_P3 (SEQ. ID NO:134), comprising an amino acid sequence being at least 70%, optionally at least about 80%, preferably at least about 85%, more preferably at least about 90% and most preferably at least about 95% homologous to the sequence GEKRDLETEVVLFHPNYNINGICKEAGIPEFYDYDVALIKLKNKLKYGQTIRPICLPCTEGTTRA LRLPPTTTCQQQKEELLPAQDIKALFVSEEEKKLTRKEVYTKNGDKKGSCERDAQYAPGYDK VKDISEVVTPRFLCTGGVSPYADPNTCRGDSGGPLIVHKRSRFIQVGVISWGVVDVCKNQKRA ALAEGETPR (SEQ. ID NO: 330) of D12115_P3 (SEQ. ID NO:134).

The localization of the variant protein was determined according to results from a number of different software programs and analyses, including analyses from SignalP and other specialized programs. The variant protein is believed to be secreted.

Variant protein D12115_P3 (SEQ. ID NO:134) also has the following non-silent SNPs (Single Nucleotide Polymorphisms) as listed in Table 29, (given according to their position(s) on the amino acid sequence, with the alternative amino acid(s) listed.

TABLE 29
Amino acid mutations

| SNP position(s) on amino acid | |
|---|---|
| sequence | Alternative amino acid(s) |
| 9 | L -> H |
| 32 | R -> Q |
| 32 | R -> W |
| 118 | S -> A |
| 142 | N -> I |
| 225 | D -> |
| 225 | D -> E |
| 252 | G -> S |
| 254 | G -> |
| 365 | M -> I |
| 428 | F -> |
| 556 | H -> Q |
| 565 | K -> E |
| 598 | P -> A |
| 598 | P -> S |
| 603 | T -> |
| 651 | D -> E |
| 677 | T -> |
| 693 | N -> |
| 729 | K -> R |
| 733 | L -> |

The glycosylation sites of variant protein D12115_P3 (SEQ. ID NO:134), as compared to the known protein Complement factor B precursor (SEQ. ID NO:131), are described in Table 30 (given according to their position(s) on the amino acid sequence in the first column; the second column indicates whether the glycosylation site is present in the variant protein; and the last column indicates whether the position is different on the variant protein).

TABLE 30
Glycosylation site(s)

| Position(s) on known | Present in | Position(s) |
|---|---|---|
| amino acid sequence | variant protein? | on variant protein |
| 122 | Yes | 122 |
| 142 | Yes | 142 |
| 285 | Yes | 285 |
| 291 | Yes | 291 |
| 378 | Yes | 378 |

The variant protein has the following domains, as determined by using InterPro. The domains are described in Table 31:

TABLE 31
InterPro domain(s)

| | Analysis | |
|---|---|---|
| Domain description | type | Position(s) on protein |
| von Willebrand factor, type A | FPrintScan | 269-286, 308-322, 383-391 |
| Peptidase S1A, chymotrypsin | FPrintScan | 512-527, 572-586, 692-704 |
| Sushi | HMMPfam | 37-86, 103-158, 165-218 |
| Peptidase S1, chymotrypsin | HMMPfam | 481-734 |
| von Willebrand factor, type A | HMMPfam | 270-468 |
| Peptidase S1, chymotrypsin | HMMSmart | 481-726 |
| Sushi | HMMSmart | 37-89, 103-158, 165-218 |
| von Willebrand factor, type A | HMMSmart | 268-473 |
| von Willebrand factor, type A | ProfileScan | 270-469 |
| Peptidase S1, chymotrypsin | ProfileScan | 477-740 |
| Peptidase S1, chymotrypsin | ScanRegExp | 522-527 |
| Peptidase S1, chymotrypsin | ScanRegExp | 693-704 |

Variant protein D12115_P3 (SEQ. ID NO:134) is encoded by the following transcript(s): D12115_T3 (SEQ. ID NO:78), for which the coding portion starts at position 514 and ends at position 2733. The transcript also has the following SNPs as listed in Table 32 (given according to their position on the nucleotide sequence, with the alternative nucleic acid listed.

TABLE 32
Nucleic acid SNPs

| SNP position(s) on nucleotide | |
|---|---|
| sequence | Alternative nucleic acid(s) |
| 325 | G -> |
| 539 | T -> A |
| 607 | C -> T |
| 608 | G -> A |
| 865 | T -> G |
| 918 | C -> T |
| 938 | A -> T |
| 963 | G -> A |
| 1017 | G -> A |
| 1185 | C -> T |
| 1188 | C -> A |
| 1188 | C -> |
| 1267 | G -> A |
| 1273 | G -> |
| 1608 | G -> T |
| 1795 | T -> |
| 1878 | C -> T |
| 1894 | C -> T |
| 2181 | C -> A |
| 2184 | C -> T |
| 2206 | A -> G |
| 2305 | C -> G |
| 2305 | C -> T |
| 2320 | A -> |
| 2439 | G -> A |
| 2466 | T -> G |
| 2543 | C -> |
| 2591 | A -> |
| 2619 | C -> T |
| 2699 | A -> G |
| 2710 | C -> |
| 2729 | -> C |
| 2778 | C -> T |

Variant protein D12115_P5 (SEQ. ID NO:135) according to the present invention is encoded by transcripts D12115_T36 (SEQ. ID NO:88) and D12115_T5 (SEQ. ID NO:79). One or more alignments to one or more previously published Complement factor B precursor (SEQ. ID NO:131) protein sequences are given in the alignment table on the attached CD-ROM. A brief description of the relationship of the variant protein according to the present invention to each such aligned protein is as follows:

### 1. Comparison Report Between D12115_P5 (SEQ. ID NO:135) and CFAB_HUMAN (SEQ. ID NO: 395):

A. An isolated chimeric polypeptide encoding for D12115_P5 (SEQ. ID NO:135), comprising a first amino acid sequence being at least 90% homologous to MGSNLSPQLCLMPFELGLLSGGVTTTPWSLARPQGSCSLEGVEIKGGSFRLLQEGQALEYVCPS GFYPYPVQTRTCRSTGSWSTLKTQDQKTVRKAECRAIHCPRPHDFENGEYWPRSPYYNVSDE ISFHCYDGYTLRGSANRTCQVNGRWSGQTAICDNGAGYCSNPGIPIGTRKVGSQYRLEDSVT YHCSRGLTLRGSQRRTCQEGGSWSGTEPSCQDSFMYDTPQEVAEAFLSSLTETIEGVDAEDGH GPGEQQKRICIVLDPSGSMNIYLVLDGSDSIGASNFTGAKKCLVNLIEKVASYGVKPRYGLVTY ATYPKIWVKVSEADSSNADVVVTKQLNEINYEDFIKLKSGTNTKKALQAVYSMMSWPDDVPP EGWNRTRHVIILMTDG corresponding to amino acids 1-390 of CFAB_HUMAN (SEQ. ID NO: 395), which also corresponds to amino acids 1-390 of D12115_P5 (SEQ. ID NO:135), and a second amino acid sequence being at least 70%, optionally at least 80%, preferably at least 85%, more preferably at least 90% and most preferably at least 95% homologous to a polypeptide having the sequence QKGPLSCPSLPTFSDQHVALKSTCNTIPMVGALNVTHSWLFISPVTLHKEFFLSPVINYL (SEQ. ID NO: 331) corresponding to amino acids 391-450 of D12115_P5 (SEQ. ID NO:135), wherein said first amino acid sequence and second amino acid sequence are contiguous and in a sequential order.

B. An isolated polypeptide encoding for an edge portion of D12115_P5 (SEQ. ID NO:135), comprising an amino acid sequence being at least 70%, optionally at least about 80%, preferably at least about 85%, more preferably at least about 90% and most preferably at least about 95% homologous to the sequence QKGPLSCPSLPTFSDQHVALKSTCNTIPMVGALNVTHSWLFISPVTLIIKEFFLSPVINYL (SEQ. ID NO: 331) of D12115_P5 (SEQ. ID NO:135).

### 2. Comparison Report Between D12115_P5 (SEQ. ID NO:135) and P00751-2 (SEQ. ID NO:132):

A. An isolated chimeric polypeptide encoding for D12115_P5 (SEQ. ID NO:135), comprising a first amino acid sequence being at least 90% homologous to MGSNLSPQLCLMPFILGLLSGGVTITPWSLARPQGSCSLEGVEIKGGSFRLLQEGQALEYVCPS GFYPYPVQTRTCRSTGSWSTLKTQDQKTVRKAECRAIHCPRPHDFENGEYWPRSPYYNVSDE ISFHCYDGYTLRGSANRTCQVNGRWSGQTAICDNGAGYCSNPGIPIGTRKVGSQYRLEDSVT YHCSRGLTLRGSQRRTCQEGGSWSGTEPSCQDSFMYDTPQEVAEAFLSSLTETIEGVDAEDGH GPGEQQKRICIVLDPSGSMNEYLVLDGSDSIGASNFTGAICKCINNLIEKVASYGVKPRYGLVTY ATYPKIWVKVSEADSSNADWVTKQLNEINYEDFEKLKSGTNTKKALQAVYSMMSWPDDVPP EGWNRTRHVIILMTDG corresponding to amino acids 1-390 of P00751-2 (SEQ. ID NO:132), which also corresponds to amino acids 1-390 of D12115_P5 (SEQ. ID NO:135), and a second amino acid sequence being at least 70%, optionally at least 80%, preferably at least 85%, more preferably at least 90% and most preferably at least 95% homologous to a polypeptide having the sequence QKGPLSCPSLPTFSDQHVALKSTCNTIPMVGALINIVTHSWLFISPVTLIIKEFFLSPVINYL (SEQ. ID NO: 331) corresponding to amino acids 391-450 of D12115_P5 (SEQ. ID NO:135), wherein said first amino acid sequence and second amino acid sequence are contiguous and in a sequential order.

B. An isolated polypeptide encoding for an edge portion of D12115_P5 (SEQ. ID NO:135), comprising an amino acid sequence being at least 70%, optionally at least about 80%, preferably at least about 85%, more preferably at least about 90% and most preferably at least about 95% homologous to the sequence QKGPLSCPSLPTFSDQHVALKSTCNTIPMVGALNVTHSVVLFISPVTLHKEFFLSPVINYL (SEQ. ID NO: 331) of D12115_P5 (SEQ. ID NO:135).

### 3. Comparison Report Between D12115_P5 (SEQ. ID NO:135) and NP_{—}001701 (SEQ. ID NO:133):

A. An isolated chimeric polypeptide encoding for D12115_P5 (SEQ. ID NO:135), comprising a first amino acid sequence being at least 90% homologous to MGSNLSPQLCLMPFILGLLSGGVTTTPWSLA corresponding to amino acids 1-31 of NP_{—}001701 (SEQ. ID NO:133), which also corresponds to amino acids 1-31 of D12115_P5 (SEQ. ID NO:135), a bridging amino acid R corresponding to amino acid 32 of D12115_P5 (SEQ. ID NO:135), a second amino acid sequence being at least 90% homologous to PQGSCSLEGVEIKGGSFRLLQEGQALEYVCPSGFYPYPVQTRTCRSTGSWSTLKTQDQKTVRK AECRAINCPRPHDFENGEYWPRSPYYNVSDEISFHCYDGYTLRGSANRTCQVNGRWSGQTAI CDNGAGYCSNPGIPIGTRKVGSQYRLEDSVTYHCSRGLTLRGSQRRTCQEGGSWSGTEPSCQ DSFMYDTPQEVAEAFLSSLTETIEGVDAEDGHGPGEQQKRKIVLDPSGSMNIYLVLDGSDSIG ASNFTGAKKCLVNLIEKVASYGVKPRYGLVITATYPICIWVKVSEADSSNADWVTKQLNEIN YEDHKLKSGTNTKKALQAVYSMMSWPDDVPPEGWNRTRHVIILMTDG corresponding to amino acids 33-390 of NP_{—}001701 (SEQ. ID NO:133), which also corresponds to amino acids 33-390 of D121151³5 (SEQ. ID NO:135), and a third amino acid sequence being at least 70%, optionally at least 80%, preferably at least 85%, more preferably at least 90% and most preferably at least 95% homologous to a polypeptide having the sequence QKGPLSCPSLPTFSDQHVALKSTCNTIPMVGALNVTHSWLFISPVTLHKEFFLSPVINYL (SEQ. ID NO: 331) corresponding to amino acids 391-450 of D12115_P5 (SEQ. ID NO:135), wherein said first amino acid sequence, bridging amino acid, second amino acid sequence and third amino acid sequence are contiguous and in a sequential order.

B. An isolated polypeptide encoding for an edge portion of D12115_P5 (SEQ. ID NO:135), comprising an amino acid sequence being at least 70%, optionally at least about 80%, preferably at least about 85%, more preferably at least about 90% and most preferably at least about 95% homologous to the sequence QKGPLSCPSLPTFSDQHVALKSTCNTIPMVGALNVTHSWLFISPVTLHKEFFLSPVINYL (SEQ. ID NO: 331) of D12115_P5 (SEQ. ID NO:135).

The localization of the variant protein was determined according to results from a number of different software programs and analyses, including analyses from SignalP and other specialized programs. The variant protein is believed to be secreted.

Variant protein D12115_P5 (SEQ. ID NO:135) also has the following non-silent SNPs (Single Nucleotide Polymorphisms) as listed in Table 33, (given according to their position(s) on the amino acid sequence, with the alternative amino acid(s) listed.

TABLE 33
Amino acid mutations

| SNP position(s) on amino acid | |
|---|---|
| sequence | Alternative amino acid(s) |
| 9 | L -> H |
| 32 | R -> Q |
| 32 | R -> W |
| 118 | S -> A |
| 142 | N -> I |
| 225 | D -> |
| 225 | D -> E |
| 252 | G -> S |
| 254 | G -> |
| 365 | M -> I |
| 449 | Y -> C |

The glycosylation sites of variant protein D12115_P5 (SEQ. ID NO:135), as compared to the known protein Complement factor B precursor (SEQ. ID NO:131), are described in Table 34 (given according to their position(s) on the amino acid sequence in the first column; the second column indicates whether the glycosylation site is present in the variant protein; and the last column indicates whether the position is different on the variant protein).

TABLE 34
Glycosylation site(s)

| Position(s) on known | Present in | Position(s) |
|---|---|---|
| amino acid sequence | variant protein? | on variant protein |
| 122 | Yes | 122 |
| 142 | Yes | 142 |
| 285 | Yes | 285 |
| 291 | Yes | 291 |
| 378 | Yes | 378 |

The variant protein has the following domains, as determined by using InterPro. The domains are described in Table 35:

TABLE 35
InterPro domain(s)

| | Analysis | |
|---|---|---|
| Domain description | type | Position(s) on protein |
| von Willebrand factor, type A | FPrintScan | 269-286, 308-322, 383-391 |
| Sushi | HMMPfam | 37-86, 103-158, 165-218 |
| von Willebrand factor, type A | HMMPfam | 270-435 |
| Sushi | HMMSmart | 37-89, 103-158, 165-218 |
| von Willebrand factor, type A | HMMSmart | 268-436 |
| von Willebrand factor, type A | ProfileScan | 270-391 |

Variant protein D12115_P5 (SEQ. ID NO:135) is encoded by the following transcript(s): D12115_T36 (SEQ. ID NO:88) and D12115_T5 (SEQ. ID NO:79), for which the coding portion starts at position 514 and ends at position 1863. The transcript also has the following SNPs as listed in Table 36 (given according to their position on the nucleotide sequence, with the alternative nucleic acid listed.

TABLE 36
Nucleic acid SNPs

| SNP position(s) on nucleotide | |
|---|---|
| sequence | Alternative nucleic acid(s) |
| 325 | G -> |
| 539 | T -> A |
| 607 | C -> T |
| 608 | G -> A |
| 865 | T -> G |
| 918 | C -> T |
| 938 | A -> T |
| 963 | G -> A |
| 1017 | G -> A |
| 1185 | C -> T |
| 1188 | C -> A |
| 1188 | C -> |
| 1267 | G -> A |
| 1273 | G -> |
| 1608 | G -> T |
| 1859 | A -> G |
| 1894 | T -> C |
| 1928 | T -> A |
| 2076 | T -> |
| 2159 | C -> T |
| 2175 | C -> T |
| 2209 | A -> C |

The coding portion of transcript D12115_T5 (SEQ. ID NO:79) starts at position 514 and ends at position 1863. The transcript also has the following SNPs as listed in Table 37 (given according to their position on the nucleotide sequence, with the alternative nucleic acid listed.

TABLE 37
Nucleic acid SNPs

| SNP position(s) on nucleotide | |
|---|---|
| sequence | Alternative nucleic acid(s) |
| 325 | G -> |
| 539 | T -> A |
| 607 | C -> T |
| 608 | G -> A |
| 865 | T -> G |
| 918 | C -> T |
| 938 | A -> T |
| 963 | G -> A |
| 1017 | G -> A |
| 1185 | C -> T |
| 1188 | C -> A |
| 1188 | C -> |
| 1267 | G -> A |
| 1273 | G -> |
| 1608 | G -> T |
| 1859 | A -> G |
| 1894 | T -> C |
| 1928 | T -> A |
| 2076 | T -> |
| 2159 | C -> T |
| 2175 | C -> T |
| 2462 | C -> A |
| 2465 | C -> T |
| 2487 | A -> G |
| 2586 | C -> G |
| 2586 | C -> T |
| 2601 | A -> |
| 2720 | G -> A |
| 2747 | T -> G |
| 2824 | C -> |
| 2872 | A -> |
| 2900 | C -> T |
| 2980 | A -> G |
| 3044 | C -> |
| 3063 | -> C |
| 3112 | C -> T |

Variant protein D12115_P12 (SEQ. ID NO:136) according to the present invention is encoded by transcript D12115_T12 (SEQ. ID NO:81). One or more alignments to one or more previously published Complement factor B precursor (SEQ. ID NO:131) protein sequences are given in the alignment table on the attached CD-ROM. A brief description of the relationship of the variant protein according to the present invention to each such aligned proteins is as follows:

### 1. Comparison Report Between D12115_P12 (SEQ. ID NO:136) and CFAB_HUMAN (SEQ. ID NO: 395):

A. An isolated chimeric polypeptide encoding for D12115_P12 (SEQ. ID NO:136), comprising a first amino acid sequence being at least 90% homologous to MGSNLSPQLCLMPFILGLLSGGVTTTPWSLARPQGSCSLEGVEIKGGSFRLLQEGQALEYVCPS GFYPYPVQTRTCRSTGSWSTLKTQDQKTVRKAECRAIHCPRPHDFENGEYWPRSPYYNVSDE ISFHCYDGYTLRGSANRICQVNGRWSGQTAICDNGAGYCSNPGIPIGTRICVGSQYRLEDSVT YHCSRGLTLRGSQRRTCQEGGSWSGTEPSCQDSFMYDTPQEVAEAFLSSLTETLEGVDAEDGH GPGEQQKRKIVLDPSGSIVJNIYLVLDGSDSIGASNFTGAKKCLVNLIEKVASYGVKPRYGLVTY ATYPKIWVKVSEADSSNADWVTKQLNEINYEDHKLKSGTNTKKALQAVYSMMSWPDDVPP EGWNRTRHVIILMTDGLHNMGGDPITVIDEIRDLLYIGKDRKNPREDYLDVYVEGVGPLVNQ VNINALASICKDNEQHVFKVICDMENLEDVEYQMDDESQSLSLCGMVWEHRKGTDYHKQPWQ AKISVIRPSKGRESCMGAVVSEYFVLTAAHCFTVDDKEHSIKVSVGGEKRDLEIEVVLFHPNY NINGICKEAGIPEFYDYDVALIKLKNICLKYGQIIRPICLPCTEGTIRALRLPPTTTCQQQKEELL PAQDIKALFVSEEEKICLTRKEVYIKNGDKKGSCERDAQYAPGYDKVICDISEVVTPRFLCTGG VSPYADPNTCRGDSGGPLIVHKRSRFIQV corresponding to amino acids 1-714 of CFAB_HUMAN (SEQ. ID NO: 395), which also corresponds to amino acids 1-714 of D12115_P12 (SEQ. ID NO:136), and a second amino acid sequence being at least 70%, optionally at least 80%, preferably at least 85%, more preferably at least 90% and most preferably at least 95% homologous to a polypeptide having the sequence SPPFPIWGDAKWSAWAPKQESSMHVASNSR (SEQ. ID NO: 332) corresponding to amino acids 715-744 of D12115_P12 (SEQ. ID NO:136), wherein said first amino acid sequence and second amino acid sequence are contiguous and in a sequential order.

B. An isolated polypeptide encoding for an edge portion of D12115_P12 (SEQ. ID NO:136), comprising an amino acid sequence being at least 70%, optionally at least about 80%, preferably at least about 85%, more preferably at least about 90% and most preferably at least about 95% homologous to the sequence SPPFPIWGDAKWSAWAPKQESSMHVASNSR (SEQ. ID NO: 332) of D12115_P12 (SEQ. ID NO:136).

### 2. Comparison Report Between D12115_P12 (SEQ. ID NO:136) and NP_{—}001701 (SEQ. ID NO:133):

A. An isolated chimeric polypeptide encoding for D12115_P12 (SEQ. ID NO:136), comprising a first amino acid sequence being at least 90% homologous to MGSNLSPQLCLMPFILGLLSGGVTTTPWSLA corresponding to amino acids 1-31 of NP_{—}001701 (SEQ. ID NO:133), which also corresponds to amino acids 1-31 of D12115_P12 (SEQ. ID NO:136), a bridging amino acid R corresponding to amino acid 32 of D12115_P12 (SEQ. ID NO:136), a second amino acid sequence being at least 90% homologous to PQGSCSLEGVEIKGGSFRLLQEGQALEYVCPSGFYPYPVQTRTCRSTGSWSTLKTQDQKTVRIC AECRAIHCPRPHDFENGEYWPRSPYYNVSDEISFHCYDGYTLRGSANRTCQVNGRWSGQTAI CDNGAGYCSNPGIPIGTRKVGSQYRLEDSVTYHCSRGLTLRGSQRRTCQEGGSWSGTEPSCQ DSFMYDTPQEVAEAFLSSLTETIEGVDAEDGHGPGEQQKRKIVLDPSGSMNIYLVLDGSDSIG ASNFTGAKKCLVNLIEKVASYGVKPRYGLVTYATYPKIWVKVSEADSSNADWVTKQLNEIN YEDHKLKSGTNTICKALQAVYSMMSWPDDVPPEGWNRTRHVIILMTDGIIINMGGDPITVIDE IRDLLYIGKDRKNPREDYLDVYVFGVGPLVNQVNINALASKKDNEQHVFKVKDMENLEDVF YQMIDESQSLSLCGMVWEHRKGTDYHKQPWQAKISVIRPSKGHESCMGAVVSEYFVLTAAH CFTVDDKEHSIKVSVGGEICRDLEIEVVLFHPNYNINGKKEAGlPEFYDYDVALIKLICNICLKYG QTIRPICLPCTEGTTRALRLPPTITCQQQICEELLPAQDIKALFVSEEEICKLTRKEVYIKNGDICK GSCERDAQYAPGYDKVKDISEVVTPRFLCTGGVSPYADPNTCRGDSGGPLIVHKRSRFIQV corresponding to amino acids 33-714 of NP_{—}001701 (SEQ. ID NO:133), which also corresponds to amino acids 33-714 of D12115_P12 (SEQ. ID NO:136), and a third amino acid sequence being at least 70%, optionally at least 80%, preferably at least 85%, more preferably at least 90% and most preferably at least 95% homologous to a polypeptide having the sequence SPPFPIWGDAKWSAWAPKQESSMHVASNSR (SEQ. ID NO: 332) corresponding to amino acids 715-744 of D12115_P12 (SEQ. ID NO:136), wherein said first amino acid sequence, bridging amino acid, second amino acid sequence and third amino acid sequence are contiguous and in a sequential order.

B. An isolated polypeptide encoding for an edge portion of D12115_P12 (SEQ. ID NO:136), comprising an amino acid sequence being at least 70%, optionally at least about 80%, preferably at least about 85%, more preferably at least about 90% and most preferably at least about 95% homologous to the sequence SPPFPIWGDAKWSAWAPKQESSMHVASNSR (SEQ. ID NO: 332) of D12115_P12 (SEQ. ID NO:136).

### 3. Comparison Report Between D12115_P12 (SEQ. ID NO:136) and P00751-2 (SEQ. ID NO:132):

A. An isolated chimeric polypeptide encoding for D12115_P12 (SEQ. ID NO:136), comprising a first amino acid sequence being at least 90% homologous to MGSNLSPQLCLMPFILGLLSGGVTITPWSLARPQGSCSLEGVEIKGGSFRLLQEGQALEYVCPS GFYPYPVQTRTCRSTGSWSTLKTQDQKTVRKAECRAIHCPRPHDFENGEYWPRSPYYNVSDE ISFHCYDGYTLRGSANRTCQVNGRWSGQTAICDNGAGYCSNPGIPIGTRKVGSQYRLEDSVT YHCSRGLTLRGSQRRTCQEGGSWSGTEPSCQDSFMYDTPQEVAEAFLSSLTETIEGVDAEDGH GPGEQQICRKIVLDPSGSMNIYLVLDGSDSIGASNFTGAKKCLVNLIEKVASYGVKPRYGLVTY ATYPKIWVKVSEADSSNADWVTKQLNEINYEDHICLKSGTNTKICALQAVYSMNISWPDDVPP EGWNRTRHVIILMTDGLHNMGGDPITVIDEIRDLLYIGKDRICNPREDYLDVYVFGVGPLVNQ VNINALASKKDNEQHVFKVICDMENLEDVFYQMIDESQSLSLCGMVWEHRKGTDYRKQPWQ AKISVIRPSKGHESCMGAVVSEYFVLTAAHCFTVDDKEHSIKVSVG corresponding to amino acids 1-542 of P00751-2 (SEQ. ID NO:132), which also corresponds to amino acids 1-542 of D12115_P12 (SEQ. ID NO:136), and a second amino acid sequence being at least 70%, optionally at least 80%, preferably at least 85%, more preferably at least 90% and most preferably at least 95% homologous to a polypeptide having the sequence GEKRDLEIEVVLFHPNYNINGKKEAGIPEFYDYDVALICKLKNKLKYGQTIRPICLPCTEGTTRA LRLPFITTCQQQKEELLPAQDIKALFVSEEEKKLTRKEVYIKNGDKKGSCERDAQYAPGYDK VKDISEVVTPRFLCTGGVSPYADPNTCRGDSGGPLIVHKRSRFIQVSPPFPIWGDAKWSAWAP KQESSMHVASNSR (SEQ. ID NO: 333) corresponding to amino acids 543-744 of D12115_P12 (SEQ. ID NO:136), wherein said first amino acid sequence and second amino acid sequence are contiguous and in a sequential order.

B. An isolated polypeptide encoding for an edge portion of D12115_P12 (SEQ. ID NO:136), comprising an amino acid sequence being at least 70%, optionally at least about 80%, preferably at least about 85%, more preferably at least about 90% and most preferably at least about 95% homologous to the sequence GEKRDLELEVVLFHPNYNINGICKEAGIPEFYDYDVALEKLICNKLKYGQTIRPICLPCTEGTTRA LRLPPITTCQQQICEELLPAQDIKALFVSEEEKKLTRKEVYIKNGDICKGSCERDAQYAPGYDK VKDISEVVTPRFLCTGGVSPYADPNTCRGDSGGPLIVHKRSRFIQVSPPFPIWGDAKWSAWAP KQESSMHVASNSR (SEQ. ID NO: 333) of D12115_P12 (SEQ. ID NO:136).

The localization of the variant protein was determined according to results from a number of different software programs and analyses, including analyses from SignalP and other specialized programs. The variant protein is believed to be secreted.

Variant protein D12115_P12 (SEQ. ID NO:136) also has the following non-silent SNPs (Single Nucleotide Polymorphisms) as listed in Table 38, (given according to their position(s) on the amino acid sequence, with the alternative amino acid(s) listed.

TABLE 38
Amino acid mutations

| SNP position(s) on amino acid | |
|---|---|
| sequence | Alternative amino acid(s) |
| 9 | L -> H |
| 32 | R -> Q |
| 32 | R -> W |
| 118 | S -> A |
| 142 | N -> I |
| 225 | D -> |
| 225 | D -> E |
| 252 | G -> S |
| 254 | G -> |
| 365 | M -> I |
| 428 | F -> |
| 556 | H -> Q |
| 565 | K -> E |
| 598 | P -> A |
| 598 | P -> S |
| 603 | T -> |
| 651 | D -> E |
| 677 | T -> |
| 693 | N -> |

The glycosylation sites of variant protein D12115_P12 (SEQ. ID NO:136), as compared to the known protein Complement factor B precursor (SEQ. ID NO:131), are described in Table 39 (given according to their position(s) on the amino acid sequence in the first column; the second column indicates whether the glycosylation site is present in the variant protein; and the last column indicates whether the position is different on the variant protein).

TABLE 39
Glycosylation site(s)

| Position(s) on known | Present in | Position(s) |
|---|---|---|
| amino acid sequence | variant protein? | on variant protein |
| 122 | Yes | 122 |
| 142 | Yes | 142 |
| 285 | Yes | 285 |
| 291 | Yes | 291 |
| 378 | Yes | 378 |

The variant protein has the following domains, as determined by using InterPro. The domains are described in Table 40:

TABLE 40
InterPro domain(s)

| | Analysis | |
|---|---|---|
| Domain description | type | Position(s) on protein |
| von Willebrand factor, type A | FPrintScan | 269-286, 308-322, 383-391 |
| Peptidase S1A, chymotrypsin | FPrintScan | 512-527, 572-586, 692-704 |
| Sushi | HMMPfam | 37-86, 103-158, 165-218 |
| Peptidase S1, chymotrypsin | HMMPfam | 481-730 |
| von Willebrand factor, type A | HMMPfam | 270-468 |
| Peptidase S1, chymotrypsin | HMMSmart | 481-720 |
| Sushi | HMMSmart | 37-89, 103-158, 165-218 |
| von Willebrand factor, type A | HMMSmart | 268-473 |
| von Willebrand factor, type A | ProfileScan | 270-469 |
| Peptidase S1, chymotrypsin | ProfileScan | 477-744 |
| Peptidase S1, chymotrypsin | ScanRegExp | 522-527 |
| Peptidase S1, chymotrypsin | ScanRegExp | 693-704 |

Variant protein D12115_P12 (SEQ. ID NO:136) is encoded by the following transcript(s): D12115_T12 (SEQ. ID NO:81), for which the coding portion starts at position 514 and ends at position 2745. The transcript also has the following SNPs as listed in Table 41 (given according to their position on the nucleotide sequence, with the alternative nucleic acid listed.

TABLE 41
Nucleic acid SNPs

| SNP position(s) on nucleotide | |
|---|---|
| sequence | Alternative nucleic acid(s) |
| 325 | G -> |
| 539 | T -> A |
| 607 | C -> T |
| 608 | G -> A |
| 865 | T -> G |
| 918 | C -> T |
| 938 | A -> T |
| 963 | G -> A |
| 1017 | G -> A |
| 1185 | C -> T |
| 1188 | C -> A |
| 1188 | C -> |
| 1267 | G -> A |
| 1273 | G -> |
| 1608 | G -> T |
| 1795 | T -> |
| 1878 | C -> T |
| 1894 | C -> T |
| 2181 | C -> A |
| 2184 | C -> T |
| 2206 | A -> G |
| 2305 | C -> G |
| 2305 | C -> T |
| 2320 | A -> |
| 2439 | G -> A |
| 2466 | T -> G |
| 2543 | C -> |
| 2591 | A -> |
| 2619 | C -> T |
| 2849 | G -> A |
| 2969 | A -> G |
| 3033 | C -> |
| 3052 | -> C |
| 3101 | C -> T |

Variant protein D12115_P13 (SEQ. ID NO:137) according to the present invention is encoded by transcript D12115_T13 (SEQ. ID NO:82). One or more alignments to one or more previously published Complement factor B precursor (SEQ. ID NO:131) protein sequences are given in the alignment table on the attached CD-ROM. A brief description of the relationship of the variant protein according to the present invention to each such aligned protein is as follows:

### 1. Comparison Report Between D12115_P13 (SEQ. ID NO:137) and CFAB_HUMAN (SEQ. ID NO: 395):

A. An isolated chimeric, polypeptide encoding for D12115_P13 (SEQ. ID NO:137), comprising a first amino acid sequence being at least 90% homologous to MGSNLSPQLCLMPFILGLLSGGVTTTPWSLARPQGSCSLEGVEIKGGSFRLLQEGQALEYVCPS GFYPYPVQTRTCRSTGSWSTLKTQDQKTVRKAECRAIRCPRPHDFENGEYWPRSPYYNVSDE ISFHCYDGYTLRGSANRTCQVNGRWSGQTAICDNGAGYCSNPGIPIGTRKVGSQYRLEDSVT YHCSRGLTLRGSQRRTCQEGGSWSGTEPSCQDSFMYDTPQEVAEAFLSSLTEITEGVDAEDGH GPGEQQKRKIVLDPSGSMINTYLVLDGSDSIGASNFTGAKKCLVNLIEKVASYGVKPRYGLVTY ATYPKIWVKVSEADSSNADWVTKQLNEINYEDHKLKSGTNTICKALQAVYSMMSWPDDVPP EGWNRTRHVIILMTDGLIINMGGDPITVIDEIRDLLYIGKDRKNPREDYLDVYVFGVGPLVNQ VNINALASKICDNEQHVFKVKDMENLEDVFYQMIDESQSLSLCGMVWEHRKGTDYFIKQPWQ AKISVIRPSKGHESCMGAVVSEYFVLTAAHCFTVDDKEHSIKVSVGGEKRDLEIEVVLFHPNY NINGKKEAGIPEFYDYDVALIKLKNKLKYGQTIRPICLPCTEGTTRALRLPPTTTCQQQ corresponding to amino acids 1-618 of CFAB_HUMAN (SEQ. ID NO: 395), which also corresponds to amino acids 1-618 of D12115_P13 (SEQ. ID NO:137), and a second amino acid sequence being at least 70%, optionally at least 80%, preferably at least 85%, more preferably at least 90% and most preferably at least 95% homologous to a polypeptide having the sequence RRAAPCTGYQSSVCV (SEQ. ID NO: 334) corresponding to amino acids 619-633 of D12115_P13 (SEQ. ID NO:137), wherein said first amino acid sequence and second amino acid sequence are contiguous and in a sequential order.

B. An isolated polypeptide encoding for an edge portion of D12115_P13 (SEQ. ID NO:137), comprising an amino acid sequence being at least 70%, optionally at least about 80%, preferably at least about 85%, more preferably at least about 90% and most preferably at least about 95% homologous to the sequence RRAAPCTGYQSSVCV (SEQ. ID NO: 334) of D12115_P13 (SEQ. ID NO:137).

### 2. Comparison Report Between D12115_P13 (SEQ. ID NO:137) and NP_{—}001701 (SEQ. ID NO:133):

A. An isolated chimeric polypeptide encoding for D12115_P13 (SEQ. ID NO:137), comprising a first amino acid sequence being at least 90% homologous to MGSNLSPQLCLMPFILGLLSGGVTTTPWSLA corresponding to amino acids 1-31 of NP_{—}001701 (SEQ. ID NO:133), which also corresponds to amino acids 1-31 of D12115_P13 (SEQ. ID NO:137), a bridging amino acid R corresponding to amino acid 32 of D12115_P13 (SEQ. ID NO:137), a second amino acid sequence being at least 90% homologous to PQGSCSLEGVEIKGGSFRLLQEGQALEYVCPSGFYPYPVQTRTCRSTGSWSTLKTQDQKTVRK AECRAIHCPRPHDFENGEYWPRSPYYNVSDEISFHCYDGYTLRGSANRTCQVNGRWSGQTAI CDNGAGYCSNPGIPIGTRKVGSQYRLEDSVTYHCSRGLTLRGSQRRTCQEGGSWSGTEPSCQ DSFMYDTPQEVAEAFLSSLTETIEGVDAEDGHGPGEQQKRKIVLDPSGSMNTYLVLDGSDSIG ASNFTGAKKCLVNLIEKVASYGVKPRYGLVTYATYPKIWVKVSEADSSNADWVTKQLNEIN YEDHICLKSGTNTKKALQAVYSMMSWPDDVPPEGWNRTRHVIILMTDGLHNMGGDPITVIDE IRDLLYIGKDRKNPREDYLDVYVFGVGPLVNQVNINALASKKDNEQHVFKVKDMENLEDVF YQMIDESQSLSLCGMVWEHRKGTDYHKQPWQAKISVIRPSKGRESCMGAVVSEYFVLTAAH CFTVDDKEHSEKVSVGGEKRDLETEVVLFHPNYNINGKKEAGIPEFYDYDVALIKLICNKLKYG QTIRPICLPCTEGTTRALRLPPTTTCQQQ corresponding to amino acids 33-618 of NP_{—}001701 (SEQ. ID NO:133), which also corresponds to amino acids 33-618 of D12115_P13 (SEQ. ID NO:137), and a third amino acid sequence being at least 70%, optionally at least 80%, preferably at least 85%, more preferably at least 90% and most preferably at least 95% homologous to a polypeptide having the sequence RRAAPCTGYQSSVCV (SEQ. ID NO: 334) corresponding to amino acids 619-633 of D12115_P13 (SEQ. ID NO:137), wherein said first amino acid sequence, bridging amino acid, second amino acid sequence and third amino acid sequence are contiguous and in a sequential order.

B. An isolated polypeptide encoding for an edge portion of D12115_P13 (SEQ. ID NO:137), comprising an amino acid sequence being at least 70%, optionally at least about 80%, preferably at least about 85%, more preferably at least about 90% and most preferably at least about 95% homologous to the sequence RRAAPCTGYQSSVCV (SEQ. ID NO: 334) of D12115_P13 (SEQ. ID NO:137).

### 3. Comparison Report Between D12115_P13 (SEQ. ID NO:137) and P00751-2 (SEQ. ID NO:132):

A. An isolated chimeric polypeptide encoding for D12115_P13 (SEQ. ID NO:137), comprising a first amino acid sequence being at least 90% homologous to MGSNLSPQLCLMPFILGLLSGGVTTTPWSLARPQGSCSLEGVETKGGSFRLLQEGQALEYVCPS GFYPYPVQTRTCRSTGSWSTLKTQDQKTVRICAECRAIHCPRPHDFENGEYWPRSPYYNVSDE ISFHCYDGYTLRGSANRTCQVNGRWSGQTAICDNGAGYCSNPGIPIGTRKVGSQYRLEDSVT YHCSRGLTLRGSQRRTCQEGGSWSGTEPSCQDSFMYDTPQEVAEAFLSSLTETLEGVDAEDGH GPGEQQKRKIVLDPSGSMNTYLVLDGSDSIGASNFTGAKKCLVNLIEKVASYGVKPRYGLVTY ATYPKIWVKVSEADSSNADWVTKQLNETNYEDHKLKSGTNTKKALQAVYSMMSWPDDVPP EGWNRTRHVIILMTDGLIINMGGDPITVEDEIRDLLYIGKDRKNPREDYLDVYVFGVGFLVNQ VNINALASKEDNEQHVEKVKDMENLEDVFYQMIDESQSLSLCGMVWEHRKGTDYITKQPWQ AKISVIRPSKGRESCMGAVVSEYFVLTAAHCFTVDDKEHSIKVSVG corresponding to amino acids 1-542 of P00751-2 (SEQ. ID NO:132), which also corresponds to amino acids 1-542 of D12115_P13 (SEQ. ID NO:137), and a second amino acid sequence being at least 70%, optionally at least 80%, preferably at least 85%, more preferably at least 90% and most preferably at least 95% homologous to a polypeptide having the sequence GEKRDLEIEVVLFHPNYNINGKKEAGIPEFYDYDVALIKLICNKLKYGQTIRPICLPCTEGTTRA LRLPPTTTCQQQRRAAPCTGYQSSVCV (SEQ. ID NO: 335) corresponding to amino acids 543-633 of D12115_P13 (SEQ. ID NO:137), wherein said first amino acid sequence and second amino acid sequence are contiguous and in a sequential order.

B. An isolated polypeptide encoding for an edge portion of D12115_P13 (SEQ. ID NO:137), comprising an amino acid sequence being at least 70%, optionally at least about 80%, preferably at least about 85%, more preferably at least about 90% and most preferably at least about 95% homologous to the sequence GEKRDLEIEVVLFHPNYNINGKKEAGIPEFYDYDVALIKLKNKLKYGQTIRPICLPCTEGTTRA LRLPPTTTCQQQRRAAPCTGYQSSVCV (SEQ. ID NO: 335) of D12115_P13 (SEQ. ID NO:137).

The localization of the variant protein was determined according to results from a number of different software programs and analyses, including analyses from SignalP and other specialized programs. The variant protein is believed to be secreted.

Variant protein D12115_P13 (SEQ. ID NO:137) also has the following non-silent SNPs (Single Nucleotide Polymorphisms) as listed in Table 42, (given according to their position(s) on the amino acid sequence, with the alternative amino acid(s) listed.

TABLE 42
Amino acid mutations

| SNP position(s) on amino acid | |
|---|---|
| sequence | Alternative amino acid(s) |
| 9 | L -> H |
| 32 | R -> Q |
| 32 | R -> W |
| 118 | S -> A |
| 142 | N -> I |
| 225 | D -> |
| 225 | D -> E |
| 252 | G -> S |
| 254 | G -> |
| 365 | M -> I |
| 428 | F -> |
| 556 | H -> Q |
| 565 | K -> E |
| 598 | P -> A |
| 598 | P -> S |
| 603 | T -> |

The glycosylation sites of variant protein D12115_P13 (SEQ. ID NO:137), as compared to the known protein Complement factor B precursor (SEQ. ID NO:131), are described in Table 43 (given according to their position(s) on the amino acid sequence in the first column; the second column indicates whether the glycosylation site is present in the variant protein; and the last column indicates whether the position is different on the variant protein).

TABLE 43
Glycosylation site(s)

| Position(s) on know amino | Present in | Position(s) |
|---|---|---|
| acid sequence | variant protein? | on variant protein |
| 122 | Yes | 122 |
| 142 | Yes | 142 |
| 285 | Yes | 285 |
| 291 | Yes | 291 |
| 378 | Yes | 378 |

The variant protein has the following domains, as determined by using InterPro. The domains are described in Table 44:

TABLE 44
InterPro domain(s)

| | Analysis | |
|---|---|---|
| Domain description | type | Position(s) on protein |
| von Willebrand factor, type A | FPrintScan | 269-286, 308-322, 383-391 |
| Peptidase S1A, chymotrypsin | FPrintScan | 512-527, 572-586 |
| Sushi | HMMPfam | 37-86, 103-158, 165-218 |
| Peptidase S1, chymotrypsin | HMMPfam | 481-633 |
| von Willebrand factor, type A | HMMPfam | 270-468 |
| Peptidase S1, chymotrypsin | HMMSmart | 481-633 |
| Sushi | HMMSmart | 37-89, 103-158, 165-218 |
| von Willebrand factor, type A | HMMSmart | 268-473 |
| von Willebrand factor, type A | ProfileScan | 270-469 |
| Peptidase S1, chymotrypsin | ProfileScan | 477-633 |
| Peptidase S1, chymotrypsin | ScanRegExp | 522-527 |

Variant protein D12115_P13 (SEQ. ID NO:137) is encoded by the following transcript(s): D12115_T13 (SEQ. ID NO:82), for which the coding portion starts at position 514 and ends at position 2412. The transcript also has the following SNPs as listed in Table 45 (given according to their position on the nucleotide sequence, with the alternative nucleic acid listed.

TABLE 45
Nucleic acid SNPs

| SNP position(s) on nucleotide | |
|---|---|
| sequence | Alternative nucleic acid(s) |
| 325 | G -> |
| 539 | T -> A |
| 607 | C -> T |
| 608 | G -> A |
| 865 | T -> G |
| 918 | C -> T |
| 938 | A -> T |
| 963 | G -> A |
| 1017 | G -> A |
| 1185 | C -> T |
| 1188 | C -> A |
| 1188 | C -> |
| 1267 | G -> A |
| 1273 | G -> |
| 1608 | G -> T |
| 1795 | T -> |
| 1878 | C -> T |
| 1894 | C -> T |
| 2181 | C -> A |
| 2184 | C -> T |
| 2206 | A -> G |
| 2305 | C -> G |
| 2305 | C -> T |
| 2320 | A -> |
| 2437 | G -> A |
| 2464 | T -> G |
| 2541 | C -> |
| 2589 | A -> |
| 2617 | C -> T |
| 2697 | A -> G |
| 2761 | C -> |
| 2780 | -> C |
| 2829 | C -> T |

Variant protein D12115_P15 (SEQ. ID NO:138) according to the present invention is encoded by transcript(s) D12115_T19 (SEQ. ID NO:84). One or more alignments to one or more previously published Complement factor B precursor (SEQ. ID NO:131) protein sequences are given in the alignment table on the attached CD-ROM. A brief description of the relationship of the variant protein according to the present invention to each such aligned protein is as follows:

### 1. Comparison Report Between D12115_P15 (SEQ. ID NO:138) and CFAB_HUMAN (SEQ. ID NO: 395):

A. An isolated chimeric polypeptide encoding for D12115_P15 (SEQ. ID NO:138), comprising a first amino acid sequence being at least 90% homologous to MGSNLSPQLCLMPFELGLLSGGVTTTPWSLARPQGSCSLEGVEIKGGSFRLLQEGQALEYVCPS GFYPYPVQTRTCRSTGSWSTLKTQDQKTVRICAECRAIHCPRPHDFENGEYWPRSPYYNVSDE ISFHCYDGYTLRGSANRTCQVNGRWSGQTAICDNGAGYCSNPGIPIGTRKVGSQYRLEDSVT YHCSRGLTLRGSQRRTCQEGGSWSGTEPSCQDSFMYDTPQEVAEAFLSSLTETIEGVDAEDGH GPGEQQKRKIVLDPSGSMNIYLVLDGSDSIGASNFTGAICKCLVNLIEKVASYGVKPRYGLVTY ATYPICIWVKVSEADSSNADWVTKQLNEINYEDHKLKSGTNTICKALQAVYSMMSWPDDVPP EGWNRTRHVBLMTDGLHNMGGDPITVIDEIRDLLYIGKDRKNPREDYLDVYVFGVGPLVNQ VNINALASKKDNEQHVFKVICDMENLEDVFYQMIDESQSLSLCGMVWEHRKGTDYHKQPWQ AKISVIRPSKGHESCMGAVVSEYFVLTAAHCFTVDDICEHSIKVSVGGEKRDLEIEVVLFHPNY NINGKKEAGIPEFYDYDVALIKLKNKLKYGQTIR corresponding to amino acids 1-593 of CFAB_HUMAN (SEQ. ID NO: 395), which also corresponds to amino acids 1-593 of D12115_P15 (SEQ. ID NO:138), and a second amino acid sequence being at least 70%, optionally at least 80%, preferably at least 85%, more preferably at least 90% and most preferably at least 95% homologous to a polypeptide having the sequence GRAAPCTGYQSSVCV (SEQ. ID NO: 336) corresponding to amino acids 594-608 of D12115_P15 (SEQ. ID NO:138), wherein said first amino acid sequence and second amino acid sequence are contiguous and in a sequential order.

B. An isolated polypeptide encoding for an edge portion of D12115_P15 (SEQ. ID NO:138), comprising an amino acid sequence being at least 70%, optionally at least about 80%, preferably at least about 85%, more preferably at least about 90% and most preferably at least about 95% homologous to the sequence GRAAPCTGYQSSVCV (SEQ. ID NO: 336) of D12115_P15 (SEQ. ID NO:138).

### 2. Comparison Report Between D12115_P15 (SEQ. ID NO:138) and NP_{—}001701 (SEQ. ID NO:133):

A. An isolated chimeric polypeptide encoding for D12115_P15 (SEQ. ID NO:138), comprising a first amino acid sequence being at least 90% homologous to MGSNLSPQLCLMPFILGLLSGGVTTTPWSLA corresponding to amino acids 1-31 of NP_{—}001701 (SEQ. ID NO:133), which also corresponds to amino acids 1-31 of D12115_P15 (SEQ. ID NO:138), a bridging amino acid R corresponding to amino acid 32 of D12115_P15 (SEQ. ID NO:138), a second amino acid sequence being at least 90% homologous to PQGSCSLEGVEIKGGSFRLLQEGQALEYVCPSGFYPYPVQTRTCRSTGSWSTLKTQDQKTVRK AECRAIHCPRPHDFENGEYWPRSPYYNVSDEISFHCYDGYTLRGSANRTCQVNGRWSGQTAI CDNGAGYCSNPGIPIGTRKVGSQYRLEDSVTYHCSRGLTLRGSQRRTCQEGGSWSGTEPSCQ DSFMYDTPQEVAEAFLSSLTETIEGVDAEDGHGPGEQQICRKIVLDPSGSNINTYLVLDGSDSIG ASNFTGAKKCLVNLIEKVASYGVKPRYGLVTYATYPKIWVKVSEADSSNADWVTKQLNEIN YEDHKLKSGTNTKKALQAVYSMMSWPDDVPPEGWNRTRHVIIIMTDGLHNMGGDPITVIDE IRDLLYIGKDRKNPREDYLDVYVFGVGPLVNQVNINALASKKDNEQHVFKVKDMENLEDVF YQMIDESQSLSLCGMVWEHRKGTDYBKQPWQAKISVIRPSKGFIESCMGAVVSEYFVLTAAH CFTVDDKEHSIKVSVGGEKRDLEIEVVLFHPNYNINGKKEAGIPEFYDYDVALIKLKNKLKYG QTIR corresponding to amino acids 33-593 of NP_{—}001701 (SEQ. ID NO:133), which also corresponds to amino acids 33-593 of D12115_P15 (SEQ. ID NO:138), and a third amino acid sequence being at least 70%, optionally at least 80%, preferably at least 85%, more preferably at least 90% and most preferably at least 95% homologous to a polypeptide having the sequence GRAAPCTGYQSSVCV (SEQ. ID NO: 336) corresponding to amino acids 594-608 of D12115_P15 (SEQ. ID NO:138), wherein said first amino acid sequence, bridging amino acid, second amino acid sequence and third amino acid sequence are contiguous and in a sequential order.

B. An isolated polypeptide encoding for an edge portion of D12115_P15 (SEQ. ID NO:138), comprising an amino acid sequence being at least 70%, optionally at least about 80%, preferably at least about 85%, more preferably at least about 90% and most preferably at least about 95% homologous to the sequence GRAAPCTGYQSSVCV (SEQ. ID NO: 336) of D12115_P15 (SEQ. ID NO:138).

### 3. Comparison Report Between D12115_P15 (SEQ. ID NO:138) and P00751-2 (SEQ. ID NO:132):

A. An isolated chimeric polypeptide encoding for D12115_P15 (SEQ. ID NO:138), comprising a first amino acid sequence being at least 90% homologous to MGSNLSPQLCLMPFILGLLSGGVTTTPWSLARPQGSCSLEGVEIKGGSFRLLQEGQALEYVCPS GFYPYPVQTRTCRSTGSWSTLKTQDQKTVRKAECRAIHCPRPHDFENGEYWPRSPYYNVSDE ISFHCYDGYTLRGSANRTCQVNGRWSGQTAICDNGAGYCSNPGIPIGTRKVGSQYRLEDSVT YHCSRGLTLRGSQRRTCQEGGSWSGTEPSCQDSFMYDTPQEVAEAFLSSLTETEEGVDAEDGH GPGEQQICRKIVLDPSGSIVINTYLVLDGSDSIGASNFTGAICKCLVNLIEKVASYGVICPRYGLVTY ATYPKIWVKVSEADSSNADWVTKQLNEJNYEDHKLKSGTNTKKALQAVYSMIVISWPDDVPP EGWNRTRHVDLMTDGLINMGGDPITVIDEIRDLLYIGICDRKNPREDYLDVYVFGVGPLVNQ VNINALASKKDNEQHVFKVICDMENLEDVFYQMIDESQSLSLCGMVWEHRKGTDYHKQPWQ AKISVIRPSKGHESCMGAVVSEYFVLTAAHCFTVDDKEHSIKVSVG corresponding to amino acids 1-542 of P00751-2 (SEQ. ID NO:132), which also corresponds to amino acids 1-542 of D12115_P15 (SEQ. ID NO:138), and a second amino acid sequence being at least 70%, optionally at least 80%, preferably at least 85%, more preferably at least 90% and most preferably at least 95% homologous to a polypeptide having the sequence GEKRDLELEVVLFHPNYNINGKICEAGIPEFYDYDVALIKLKNKLKYGQIIRGRAAPCTGYQSS VCV (SEQ. ID NO: 337) corresponding to amino acids 543-608 of D12115_P15 (SEQ. ID NO:138), wherein said first amino acid sequence and second amino acid sequence are contiguous and in a sequential order.

B. An isolated polypeptide encoding for an edge portion of D12115_P15 (SEQ. ID NO:138), comprising an amino acid sequence being at least 70%, optionally at least about 80%, preferably at least about 85%, more preferably at least about 90% and most preferably at least about 95% homologous to the sequence GEKRDLEIEVVLFHPNYNINGKKEAGIPEFYDYDVALIKLICNKLKYGQTERGRAAPCTGYQSS VCV (SEQ. ID NO: 337) of D12115_P15 (SEQ. ID NO:138).

The localization of the variant protein was determined according to results from a number of different software programs and analyses, including analyses from SignalP and other specialized programs. The variant protein is believed to be secreted.

Variant protein D12115_P15 (SEQ. ID NO:138) also has the following non-silent SNPs (Single Nucleotide Polymorphisms) as listed in Table 46, (given according to their position(s) on the amino acid sequence, with the alternative amino acid(s) listed.

TABLE 46
Amino acid mutations

| SNP position(s) on amino acid | |
|---|---|
| sequence | Alternative amino acid(s) |
| 9 | L -> H |
| 32 | R -> Q |
| 32 | R -> W |
| 118 | S -> A |
| 142 | N -> I |
| 225 | D -> |
| 225 | D -> E |
| 252 | G -> S |
| 254 | G -> |
| 365 | M -> I |
| 428 | F -> |
| 556 | H -> Q |
| 565 | K -> E |

The glycosylation sites of variant protein D12115_P15 (SEQ. ID NO:138), as compared to the known protein Complement factor B precursor (SEQ. ID NO:131), are described in Table 47 (given according to their position(s) on the amino acid sequence in the first column; the second column indicates whether the glycosylation site is present in the variant protein; and the last column indicates whether the position is different on the variant protein).

TABLE 47
Glycosylation site(s)

| Position(s) on known amino | Present | Position(s) |
|---|---|---|
| acid sequence | in variant protein? | on variant protein |
| 122 | Yes | 122 |
| 142 | Yes | 142 |
| 285 | Yes | 285 |
| 291 | Yes | 291 |
| 378 | Yes | 378 |

The variant protein has the following domains, as determined by using InterPro. The domains are described in Table 48:

TABLE 48
InterPro domain(s)

| | Analysis | |
|---|---|---|
| Domain description | type | Position(s) on protein |
| von Willebrand factor, type A | FPrintScan | 269-286, 308-322, 383-391 |
| Peptidase S1A, chymotrypsin | FPrintScan | 512-527, 572-586 |
| Sushi | HMMPfam | 37-86, 103-158, 165-218 |
| von Willebrand factor, type A | HMMPfam | 270-468 |
| Sushi | HMMSmart | 37-89, 103-158, 165-218 |
| von Willebrand factor, type A | HMMSmart | 268-473 |
| von Willebrand factor, type A | ProfileScan | 270-469 |
| Peptidase S1, chymotrypsin | ProfileScan | 477-602 |
| Peptidase S1, chymotrypsin | ScanRegExp | 522-527 |

Variant protein D12115_P15 (SEQ. ID NO:138) is encoded by the following transcript(s): D12115_T19 (SEQ. ID NO:84), for which the coding portion starts at position 514 and ends at position 2337. The transcript also has the following SNPs as listed in Table 49 (given according to their position on the nucleotide sequence, with the alternative nucleic acid listed.

TABLE 49
Nucleic acid SNPs

| SNP position(s) on nucleotide | |
|---|---|
| sequence | Alternative nucleic acid(s) |
| 325 | G -> |
| 539 | T -> A |
| 607 | C -> T |
| 608 | G -> A |
| 865 | T -> G |
| 918 | C -> T |
| 938 | A -> T |
| 963 | G -> A |
| 1017 | G -> A |
| 1185 | C -> T |
| 1188 | C -> A |
| 1188 | C -> |
| 1267 | G -> A |
| 1273 | G -> |
| 1608 | G -> T |
| 1795 | T -> |
| 1878 | C -> T |
| 1894 | C -> T |
| 2181 | C -> A |
| 2184 | C -> T |
| 2206 | A -> G |
| 2362 | G -> A |
| 2389 | T -> G |
| 2466 | C -> |
| 2514 | A -> |
| 2542 | C -> T |
| 2622 | A -> G |
| 2686 | C -> |
| 2705 | -> C |
| 2754 | C -> T |

Variant protein D12115_P16 (SEQ. ID NO:139) according to the present invention is encoded by transcript D12115_T22 (SEQ. ID NO:85). One or more alignments to one or more previously published Complement factor B precursor (SEQ. ID NO:131) protein sequences are given in the alignment table on the attached CD-ROM. A brief description of the relationship of the variant protein according to the present invention to each such aligned protein is as follows:

### 1. Comparison Report Between D12115_P16 (SEQ. ID NO:139) and CFAB_HUMAN (SEQ. ID NO: 395):

A. An isolated chimeric polypeptide encoding for D12115_P16 (SEQ. ID NO:139), comprising a first amino acid sequence being at least 90% homologous to MGSNLSPQLCLMPFILGLLSGGVTTTPWSLARPQGSCSLEGVETKGGSFRLLQEGQALEYVCPS GFYPYPVQTRTCRSTGSWSTLKTQDQKTVRICAECRAIHCPRPHDFENGEYWPRSPYYNVSDE ISFHCYDGYTLRGSANRTCQVNGRWSGQTAICDNGAGYCSNPGIPIGTRKVGSQYRLEDSVT YHCSRGLTLRGSQRRTCQEGGSWSGTEPSCQDSFMYDTPQEVAEAFLSSLTETIEGVDAEDGH GPGEQQICRKIVLDPSGSMNIYLVLDGSDSIGASNFTGAKKCLVNLIEKVASYGVKPRYGLVTY ATYPKIWVKVSEADSSNADWVTKQLNEINYEDHKLKSGTNTICKALQAVYSMMSWPDDVPP EGWNRTRHVIILMTDGLHNMGGDPITVBJEIRDLLYIGKDRKNPREDYLDVYVFGVGPLVNQ VNINALASICKDNEQHVFKVICDMENLEDVFYQMEDESQSLSLCGMVWEBRKGTDYHKQPWQ AKISVIRPSKGHESCMGAVVSEYFVLTAAHCFTVDDKEHSIKVSVGGEKRDLEIEVVLFHPNY NlNGKKEAGIPEFYDYDVALIKLKNKLKYGQTTRPICLPCTEGTTRALRLPPTTTCQQQKEELL PAQDIKALFVSEEEKKLTRKEVYTKNGDK corresponding to amino acids 1-652 of CFAB_HUMAN (SEQ. ID NO: 395), which also corresponds to amino acids 1-652 of D12115_P16 (SEQ. ID NO:139), and a second amino acid sequence being at least 70%, optionally at least 80%, preferably at least 85%, more preferably at least 90% and most preferably at least 95% homologous to a polypeptide having the sequence VRNGHPKEAL (SEQ. ID NO: 338) corresponding to amino acids 653-662 of D12115_P16 (SEQ. ID NO:139), wherein said first amino acid sequence and second amino acid sequence are contiguous and in a sequential order.

B. An isolated polypeptide encoding for an edge portion of D12115_P16 (SEQ. ID NO:139), comprising an amino acid sequence being at least 70%, optionally at least about 80%, preferably at least about 85%, more preferably at least about 90% and most preferably at least about 95% homologous to the sequence VRNGHPKEAL (SEQ. ID NO: 338) of D12115_{—}1³16 (SEQ. ID NO:139).

### 2. Comparison Report Between D12115_P16 (SEQ. ID NO:139) and NP_{—}001701 (SEQ. ID NO:133):

A. An isolated chimeric polypeptide encoding for D12115_P16 (SEQ. ID NO:139), comprising a first amino acid sequence being at least 90% homologous to MGSNLSPQLCLMPFILGLLSGGVTITPWSLA corresponding to amino acids 1-31 of NP_{—}001701 (SEQ. ID NO:133), which also corresponds to amino acids 1-31 of D12115_P16 (SEQ. ID NO:139), a bridging amino acid R corresponding to amino acid 32 of D12115_P16 (SEQ. ID NO:139), a second amino acid sequence being at least 90% homologous to PQGSCSLEGVELKGGSFRLLQEGQALEYVCPSGFYPYPVQTRTCRSTGSWSTLKTQDQKTVRK AECRAIRCPRPHDFENGEYWPRSPYYNVSDEISFHCYDGYTLRGSANRTCQVNGRWSGQTAI CDNGAGYCSNPGIPIGTRKVGSQYRLEDSVTYHCSRGLTLRGSQRRTCQEGGSWSGTEPSCQ DSFMYDTPQEVAEAFLSSLTETIEGVDAEDGHGPGEQQKRKIVLDPSGSMNTYLVLDGSDSIG ASNFTGAKKCLVNLIEKVASYGVKPRYGLVTYATYPKIWVKVSEADSSNADWVTKQLNEIN YEDHKLKSGTNTKKALQAVYSMMSWPDDVPPEGWNRTRHVIILMTDGLHNMGGDPITVIDE IRDLLYIGKDRKNPREDYLDVYVFGVGPLVNQVNINALASKKDNEQHVFKVKDMENLEDVF YQMIDESQSLSLCGMVWEHRKGTDYHKQPWQAKISVIRPSKGHESCMGAVVSEYFVLTAAH CFTVDDKEHSIKVSVGGEKRDLEIEVVLFHPNYNINGKKEAGIPEFYDYDVALIKLKNKLKYG QTIRPICLPCTEGTTRALRLPPTITCQQQKEELLPAQDIKALFVSEEEKKLTRKEVYIKNGDK corresponding to amino acids 33-652 of NP_{—}001701 (SEQ. ID NO:133), which also corresponds to amino acids 33-652 of D12115_P16 (SEQ. ID NO:139), and a third amino acid sequence being at least 70%, optionally at least 80%, preferably at least 85%, more preferably at least 90% and most preferably at least 95% homologous to a polypeptide having the sequence VRNGHPKEAL (SEQ. ID NO: 338) corresponding to amino acids 653-662 of D12115_P16 (SEQ. ID NO:139), wherein said first amino acid sequence, bridging amino acid, second amino acid sequence and third amino acid sequence are contiguous and in a sequential order.

B. An isolated polypeptide encoding for an edge portion of D12115_P16 (SEQ. ID NO:139), comprising an amino acid sequence being at least 70%, optionally at least about 80%, preferably at least about 85%, more preferably at least about 90% and most preferably at least about 95% homologous to the sequence VRNGHPKEAL (SEQ. ID NO: 338) of D12115_P16 (SEQ. ID NO:139).

### 3. Comparison Report Between D12115_P16 (SEQ. ID NO:139) and P00751-2 (SEQ. ID NO:132):

A. An isolated chimeric polypeptide encoding for D12115_P16 (SEQ. ID NO:139), comprising a first amino acid sequence being at least 90% homologous to MGSNLSPQLCLMPFILGLLSGGVTTTPWSLARPQGSCSLEGVEEKGGSFRLLQEGQALEYVCPS GFYPYPVQTRTCRSTGSWSTLKTQDQKTVRKAECRAIHCPRPHDFENGEYWPRSPYYNVSDE ISFHCYDGYTLRGSANRTCQVNGRWSGQTAICDNGAGYCSNPGIPIGTRKVGSQYRLEDSVT YHCSRGLTLRGSQRRTCQEGGSWSGTEPSCQDSFMYDTPQEVAEAFLSSLTETIEGVDAEDGH GPGEQQKRICIVLDPSGSMNIYLVLDGSDSIGASNFTGAKKCLVNLIEKVASYGVKPRYGLVTY ATYPKIWVKVSEADSSNADWVTKQLNEINYEDHKLKSGTNTKKALQAVYSMMSWPDDVPP EGWNRTRHVIILMTDGLHNMGGDPITVIDEIRDLLYIGKDRKNPREDYLDVYVFGVGPLVNQ VNINALASKKDNEQHVFKVKDMENLEDVFYQMIDESQSLSLCGMVWEBRKGTDYHKQPWQ AKISVIRPSKGHESCMGAVVSEYFVLTAAHCFTVDDKEHSIKVSVG corresponding to amino acids 1-542 of P00751-2 (SEQ. ID NO:132), which also corresponds to amino acids 1-542 of D12115_P16 (SEQ. ID NO:139), and a second amino acid sequence being at least 70%, optionally at least 80%, preferably at least 85%, more preferably at least 90% and most preferably at least 95% homologous to a polypeptide having the sequence GEKRDLEIEVVLFHPNYNINGKKEAGIPEFYDYDVALIICLKNKLKYGQTIRPICLPCTEGTTRA LRLPFITTCQQQKEELLPAQDIKALFVSEEEICKLTRKEVYIKNGDKVRNGHPKEAL (SEQ. ID NO: 339) corresponding to amino acids 543-662 of D12115_P16 (SEQ. ID NO:139), wherein said first amino acid sequence and second amino acid sequence are contiguous and in a sequential order.

B. An isolated polypeptide encoding for an edge portion of D12115_P16 (SEQ. ID NO:139), comprising an amino acid sequence being at least 70%, optionally at least about 80%, preferably at least about 85%, more preferably at least about 90% and most preferably at least about 95% homologous to the sequence GEKRDLEIEVVLFHPNYNINGKKEAGIPEFYDYDVALIKLKNKLKYGQIIRPICLPCTEGTTRA LRLPPTTTCQQQKEELLPAQDIKALFVSEEEKKLTRKEVYIKNGDKVRNGHPKEAL (SEQ. ID NO: 339) of D12115_P16 (SEQ. ID NO:139).

The localization of the variant protein was determined according to results from a number of different software programs and analyses, including analyses from SignalP and other specialized programs. The variant protein is believed to be secreted.

Variant protein D12115_P16 (SEQ. ID NO:139) also has the following non-silent SNPs (Single Nucleotide Polymorphisms) as listed in Table 50, (given according to their position(s) on the amino acid sequence, with the alternative amino acid(s) listed.

TABLE 50
Amino acid mutations

| SNP position(s) on amino acid | |
|---|---|
| sequence | Alternative amino acid(s) |
| 9 | L -> H |
| 32 | R -> Q |
| 32 | R -> W |
| 118 | S -> A |
| 142 | N -> I |
| 225 | D -> |
| 225 | D -> E |
| 252 | G -> S |
| 254 | G -> |
| 365 | M -> I |
| 428 | F -> |
| 556 | H -> Q |
| 565 | K -> E |
| 598 | P -> A |
| 598 | P -> S |
| 603 | T -> |
| 651 | D -> E |

The glycosylation sites of variant protein D12115_P16 (SEQ. ID NO:139), as compared to the known protein Complement factor B precursor (SEQ. ID NO:131), are described in Table 51 (given according to their position(s) on the amino acid sequence in the first column; the second column indicates whether the glycosylation site is present in the variant protein; and the last column indicates whether the position is different on the variant protein).

TABLE 51
Glycosylation site(s)

| Position(s) on known amino | Present | Position(s) |
|---|---|---|
| acid sequence | in variant protein? | on variant protein |
| 122 | Yes | 122 |
| 142 | Yes | 142 |
| 285 | Yes | 285 |
| 291 | Yes | 291 |
| 378 | Yes | 378 |

The variant protein has the following domains, as determined by using InterPro. The domains are described in Table 52:

TABLE 52
InterPro domain(s)

| | Analysis | |
|---|---|---|
| Domain description | type | Position(s) on protein |
| von Willebrand factor, type A | FPrintScan | 269-286, 308-322, 383-391 |
| Peptidase S1A, chymotrypsin | FPrintScan | 512-527, 572-586 |
| Sushi | HMMPfam | 37-86, 103-158, 165-218 |
| Peptidase S1, chymotrypsin | HMMPfam | 481-653 |
| von Willebrand factor, type A | HMMPfam | 270-468 |
| Peptidase S1, chymotrypsin | HMMSmart | 481-653 |
| Sushi | HMMSmart | 37-89, 103-158, 165-218 |
| von Willebrand factor, type A | HMMSmart | 268-473 |
| von Willebrand factor, type A | ProfileScan | 270-469 |
| Peptidase S1, chymotrypsin | ProfileScan | 477-615 |
| Peptidase S1, chymotrypsin | ScanRegExp | 522-527 |

Variant protein D12115_P16 (SEQ. ID NO:139) is encoded by the following transcript(s): D12115_T22 (SEQ. ID NO:85), for which the coding portion starts at position 514 and ends at position 2499. The transcript also has the following SNPs as listed in Table 53 (given according to their position on the nucleotide sequence, with the alternative nucleic acid listed.

TABLE 53
Nucleic acid SNPs

| SNP position(s) on nucleotide | |
|---|---|
| sequence | Alternative nucleic acid(s) |
| 325 | G -> |
| 539 | T -> A |
| 607 | C -> T |
| 608 | G -> A |
| 865 | T -> G |
| 918 | C -> T |
| 938 | A -> T |
| 963 | G -> A |
| 1017 | G -> A |
| 1185 | C -> T |
| 1188 | C -> A |
| 1188 | C -> |
| 1267 | G -> A |
| 1273 | G -> |
| 1608 | G -> T |
| 1795 | T -> |
| 1878 | C -> T |
| 1894 | C -> T |
| 2181 | C -> A |
| 2184 | C -> T |
| 2206 | A -> G |
| 2305 | C -> G |
| 2305 | C -> T |
| 2320 | A -> |
| 2439 | G -> A |
| 2466 | T -> G |
| 2639 | C -> |
| 2687 | A -> |
| 2715 | C -> T |
| 2945 | G -> A |
| 3065 | A -> G |
| 3129 | C -> |
| 3148 | -> C |
| 3197 | C -> T |

Variant protein D12115_P20 (SEQ. ID NO:140) according to the present invention is encoded by transcript D12115_T27 (SEQ. ID NO:86). One or more alignments to one or more previously published Complement factor B precursor (SEQ. ID NO:131) protein sequences are given in the alignment table on the attached CD-ROM. A brief description of the relationship of the variant protein according to the present invention to each such aligned protein is as follows:

### 1. Comparison Report Between D12115_P20 (SEQ. ID NO:140) and CFAB_HUMAN (SEQ. ID NO: 395):

A. An isolated chimeric polypeptide encoding for D12115_P20 (SEQ. ID NO:140), comprising a first amino acid sequence being at least 90% homologous to MGSNLSPQLCLMPFILGLLSGGVTTTPWSLARPQGSCSLEGVEIKGGSFRLLQEGQALEYVCPS GFYPYPVQTRTCRSTGSWSTLKTQDQKTVRKAECRAIHCPRPHDFENGEYWPRSPYYNVSDE ISFHCYDGYTLRGSANRTCQVNGRWSGQTAICDNGAGYCSNPGIPIGTRKVGSQYRLEDSVT YHCSRGLTLRGSQRRTCQEGGSWSGTEPSCQDSFMYDTPQEVAEAFLSSLTETLEGVDAEDGH GPGEQQKRKIVLDPSGSMNIYLVLDGSDSIGASNFTGAKKCLVNLIEKVASYGVKPRYGLVTY ATYPKTWVKVSEADSSNADWVTKQLNEINYEDHKLKSGINTKKALQAVYSMMSWPDDVPP EGWNRTRHVIILMTDGLHNMGGDPITVLDEIRDLLYIGICDRKNPREDYLDVYVFGVGPLVNQ VNINALASKIWNEQHVFKVKDMENLEDVFYQMIDESQSLSLCGMVWEHRKGTDYFIKQPWQ AKISVIRPSKGHESCMGAVVSEYFVLTAAHCFTVDDKEHSIKVSV corresponding to amino acids 1-541 of CFAB_HUMAN (SEQ. ID NO: 395), which also corresponds to amino acids 1-541 of D12115_P20 (SEQ. ID NO:140), a second bridging amino acid sequence comprising of E, and a third amino acid sequence being at least 90% homologous to EELLPAQDIKALFVSEEEKKLTRKEVYIKNGDKKGSCERDAQYAPGYDKVKDISEVVTPRFLC TGGVSPYADPNTCRGDSGGPLIVHKRSRFIQVGVISWGVVDVCICNQKRQKQVPAHARDFHIN LFQVLPWLKEKLQDEDLGFL corresponding to amino acids 620-764 of CFAB_HUMAN (SEQ. ID NO: 395), which also corresponds to amino acids 543-687 of D12115_P20 (SEQ. ID NO:140), wherein said first amino acid sequence, second amino acid sequence and third amino acid sequence are contiguous and in a sequential order.

B. An isolated polypeptide encoding for an edge portion of D12115_P20 (SEQ. ID NO:140), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least 3 amino acids comprise VEE having a structure as follows (numbering according to D12115_P20 (SEQ. ID NO:140)): a sequence starting from any of amino acid numbers 541−x to 541; and ending at any of amino acid numbers 543+((n−3)−x), in which x varies from 0 to n−3.

2. Comparison Report Between D12115_P20 (SEQ. ID NO:140) and NP_{—}001701 (SEQ. ID NO:133):

A. An isolated chimeric polypeptide encoding for D12115_P20 (SEQ. ID NO:140), comprising a first amino acid sequence being at least 90% homologous to MGSNLSPQLCLMPFILGLLSGGVTTTPWSLA corresponding to amino acids 1-31 of NP_{—}001701 (SEQ. ID NO:133), which also corresponds to amino acids 1-31 of D12115_P20 (SEQ. ID NO:140), a bridging amino acid R corresponding to amino acid 32 of D12115_P20 (SEQ. ID NO:140), a second amino acid sequence being at least 90% homologous to PQGSCSLEGVEIKGGSFRLLQEGQALEYVCPSGFYPYPVQTRTCRSTGSWSTLKTQDQKTVRK AECRAIHCPRPHDFENGEYWPRSPYYNVSDEISFHCYDGYTLRGSANRTCQVNGRWSGQTAI CDNGAGYCSNPGIPIGTRKVGSQYRLEDSVTYHCSRGLILRGSQRRTCQEGGSWSGTEPSCQ DSFMYDTPQEVAEAFLSSLTETIEGVDAEDGHGPGEQQKRKIVLDPSGSMNIYLVLDGSDSIG ASNFTGAKKCLVNLIEKVASYGVKPRYGLVTYATYPKIWVKVSEADSSNADWVTKQLNEIN YEDHKLKSGTNTKKALQAVYSMMSWPDDVPPEGWNRTRHVIILMTDGLHNIVIGGDPITVIDE IRDLLYIGKDRKNPREDYLDVYVFGVGPLVNQVNINALASKKDNEQHVFKVKDMENLEDVF YQMIDESQSLSLCGMVWEHRKGTDYHKQPWQAKISVIRPSKGHESCMGAVVSEYFVLTAAH CFTVDDKEHSIKVSV corresponding to amino acids 33-541 of NP_{—}001701 (SEQ. ID NO:133), which also corresponds to amino acids 33-541 of D12115220 (SEQ. ID NO:140), a third bridging amino acid sequence comprising of E, and a fourth amino acid sequence being at least 90% homologous to EELLPAQDIKALFVSEEEKKLTRKEVYIKNGDKKGSCERDAQYAPGYDKVKDISEVVTPRFLC TGGVSPYADPNTCRGDSGGPLIVHKRSRFIQVGVISWGVVDVCKNQKRQKQVPAHARDFHIN LFQVLPWLKEKLQDEDLGFL corresponding to amino acids 620-764 of NP_{—}001701 (SEQ. ID NO:133), which also corresponds to amino acids 543-687 of D12115_P20 (SEQ. ID NO:140), wherein said first amino acid sequence, bridging amino acid, second amino acid sequence, third amino acid sequence and fourth amino acid sequence are contiguous and in a sequential order.

### 3. Comparison Report Between D12115_P20 (SEQ. ID NO:140) and P00751-2 (SEQ. ID NO:132):

A. An isolated chimeric polypeptide encoding for D12115_P20 (SEQ. ID NO:140), comprising a first amino acid sequence being at least 90% homologous to MGSNLSPQLCLMPFILGLLSGGVTTTPWSLARPQGSCSLEGVEIKGGSFRLLQEGQALEYVCPS GFYPYPVQTRTCRSTGSWSTLKTQDQKTVRKAECRAIHCPRPHDFENGEYWPRSPYYNVSDE ISFHCYDGYTLRGSANRTCQVNGRWSGQTAICDNGAGYCSNPGIFIGTRKVGSQYRLEDSVT YHCSRGLTLRGSQRRTCQEGGSWSGTEPSCQDSFMYDTPQEVAEAFLSSLTETIEGVDAEDGH GPGEQQKRKIVLDPSGSMNTYLVLDGSDSIGASNFTGAKKCLVNLIEKVASYGVKPRYGLVTY ATYPKIWVKVSEADSSNADWVTKQLNEINYEDPIKLKSGTNTKKALQAVYSMMSWPDDVPP EGWNRTRHVIILMTDGLHNMGGDPITVIDEIRDLLYIGKDRKNPREDYLDVYVFGVGPLVNQ VNINALASKKDNEQHVFKVIMMENLEDVFYQMIDESQSLSLCGMVWEHRKGTDYIIKQPWQ AKISVIRPSKGHESCMGAVVSEYFVLTAAHCFTVDDKEHSEKVSV corresponding to amino acids 1-541 of P00751-2 (SEQ. ID NO:132), which also corresponds to amino acids 1-541 of D12115_P20 (SEQ. ID NO:140), and a second amino acid sequence being at least 70%, optionally at least 80%, preferably at least 85%, more preferably at least 90% and most preferably at least 95% homologous to a polypeptide having the sequence EEELLPAQDIKALFVSEEEKKLTRKEVYIKNGDKKGSCERDAQYAPGYDKVKDISEVVTPRFL CTGGVSPYADPNTCRGDSGGPLIVHKRSRFIQVGVISWGVVDVCKNQKRQKQVPAHARDFHI NLFQVLPWLKEKLQDEDLGFL (SEQ. ID NO: 340) corresponding to amino acids 542-687 of D12115_P20 (SEQ. ID NO:140), wherein said first amino acid sequence and second amino acid sequence are contiguous and in a sequential order.

B. An isolated polypeptide encoding for an edge portion of D12115_P20 (SEQ. ID NO:140), comprising an amino acid sequence being at least 70%, optionally at least about 80%, preferably at least about 85%, more preferably at least about 90% and most preferably at least about 95% homologous to the sequence EEELLPAQDIKALFVSEEEKKLTRKEVYIKNGDKKGSCERDAQYAPGYDKVKDISEVVTPRFL CTGGVSPYADPNTCRGDSGGPLIVHKRSRFIQVGVISWGVVDVCKNQKRQKQVPAHARDFHI NLFQVLPWLKEKLQDEDLGFL (SEQ. ID NO: 340) of D12115_P20 (SEQ. ID NO:140).

The localization of the variant protein was determined according to results from a number of different software programs and analyses, including analyses from SignalP and other specialized programs. The variant protein is believed to be secreted.

Variant protein D12115_P20 (SEQ. ID NO:140) also has the following non-silent SNPs (Single Nucleotide Polymorphisms) as listed in Table 54, (given according to their position(s) on the amino acid sequence, with the alternative amino acid(s) listed.

TABLE 54
Amino acid mutations

| SNP position(s) on amino acid | |
|---|---|
| sequence | Alternative amino acid(s) |
| 9 | L -> H |
| 32 | R -> Q |
| 32 | R -> W |
| 118 | S -> A |
| 142 | N -> I |
| 225 | D -> |
| 225 | D -> E |
| 252 | G -> S |
| 254 | G -> |
| 365 | M -> I |
| 428 | F -> |
| 574 | D -> E |
| 600 | T -> |
| 616 | N -> |
| 652 | K -> R |
| 673 | P -> |

The glycosylation sites of variant protein D12115_P20 (SEQ. ID NO:140), as compared to the known protein Complement factor B precursor (SEQ. ID NO:131), are described in Table 55 (given according to their position(s) on the amino acid sequence in the first column; the second column indicates whether the glycosylation site is present in the variant protein; and the last column indicates whether the position is different on the variant protein).

TABLE 55
Glycosylation site(s)

| Position(s) on known amino | Present in | Position(s) on |
|---|---|---|
| acid sequence | variant protein? | variant protein |
| 122 | Yes | 122 |
| 142 | Yes | 142 |
| 285 | Yes | 285 |
| 291 | Yes | 291 |
| 378 | Yes | 378 |

The variant protein has the following domains, as determined by using InterPro. The domains are described in Table 56:

TABLE 56
InterPro domain(s)

| Domain description | Analysis type | Position(s) on protein |
|---|---|---|
| von Willebrand factor, type A | FPrintScan | 269-286, 308-322, |
| | | 383-391 |
| Peptidase S1A, chymotrypsin | FPrintScan | 512-527, 615-627 |
| Sushi | HMMPfam | 37-86, 103-158, 165-218 |
| Peptidase S1, chymotrypsin | HMMPfam | 481-675 |
| von Willebrand factor, type A | HMMPfam | 270-468 |
| Peptidase S1, chymotrypsin | HMMSmart | 481-675 |
| Sushi | HMMSmart | 37-89, 103-158, 165-218 |
| von Willebrand factor, type A | HMMSmart | 268-473 |
| von Willebrand factor, type A | ProfileScan | 270-469 |
| Peptidase S1, chymotrypsin | ProfileScan | 477-680 |
| Peptidase S1, chymotrypsin | ScanRegExp | 522-527 |
| Peptidase S1, chymotrypsin | ScanRegExp | 616-627 |

Variant protein D12115_P20 (SEQ. ID NO:140) is encoded by the following transcript(s): D12115_T27 (SEQ. ID NO:86), for which coding portion starts at position 514 and ends at position 2574. The transcript also has the following SNPs as listed in Table 57 (given according to their position on the nucleotide sequence, with the alternative nucleic acid listed.

TABLE 57
Nucleic acid SNPs

| SNP position(s) on nucleotide | |
|---|---|
| sequence | Alternative nucleic acid(s) |
| 325 | G -> |
| 539 | T -> A |
| 607 | C -> T |
| 608 | G -> A |
| 865 | T -> G |
| 918 | C -> T |
| 938 | A -> T |
| 963 | G -> A |
| 1017 | G -> A |
| 1185 | C -> T |
| 1188 | C -> A |
| 1188 | C -> |
| 1267 | G -> A |
| 1273 | G -> |
| 1608 | G -> T |
| 1795 | T -> |
| 1878 | C -> T |
| 1894 | C -> T |
| 2208 | G -> A |
| 2235 | T -> G |
| 2312 | C -> |
| 2360 | A -> |
| 2388 | C -> T |
| 2468 | A -> G |
| 2532 | C -> |
| 2551 | -> C |
| 2600 | C -> T |

Variant protein D12115_P32 (SEQ. ID NO:141) according to the present invention is encoded by transcript D12115_T33 (SEQ. ID NO:87). One or more alignments to one or more previously published Complement factor B precursor (SEQ. ID NO:131) protein sequences are given in the alignment table on the attached CD-ROM. A brief description of the relationship of the variant protein according to the present invention to each such aligned protein is as follows:

### 1. Comparison Report Between D12115_P32 (SEQ. ID NO:141) and CFAB_HUMAN (SEQ. ID NO: 395):

A. An isolated chimeric polypeptide encoding for D12115_P32 (SEQ. ID NO:141), comprising a first amino acid sequence being at least 90% homologous to MGSNLSPQLCLMPFILGLLSGGVTTTPWSLARPQGSCSLEGVEIKGGSFRLLQEGQALEYVCPS GFYPYPVQTRTCRSTGSWSTLKTQDQKTVRKAECRAIHCPRPHDFENGEYWPRSPYYNVSDE ISFHCYDGYTLRGSANRTCQVNGRWSGQTAICDNGAGYCSNPGEPIGTRKVGSQYRLEDSVT YHCSRGLTLRGSQRRTCQEGGSWSGTEPSCQDSFMYDTPQEVAEAFLSSLTETIEGVDAEDGH GPGEQQKRKIVLDPSGSMNIYLVLDGSDSIGASNFTGAKKCLVNLIEKVASYGVKPRYGLVTY ATYPKIWVKVSEADSSNADWVTKQLNEINYEDHKLKSGTNTKKALQAVYSMMSWPDDVPP EGWNRTRHVIILMTDGLHNMGGDPITVIDEIRDLLYIGKDRKNPREDYLDVYVFGVGPLVNQ VNINALASKKDNEQHVFKVKDMENLEDVFYQMI corresponding to amino acids 1-469 of CFAB_HUMAN (SEQ. ID NO: 395), which also corresponds to amino acids 1-469 of D12115_P32 (SEQ. ID NO:141), and a second amino acid sequence being at least 70%, optionally at least 80%, preferably at least 85%, more preferably at least 90% and most preferably at least 95% homologous to a polypeptide having the sequence GREIQGNKEHNS (SEQ. ID NO: 341) corresponding to amino acids 470-481 of D12115_P32 (SEQ. ID NO:141), wherein said first amino acid sequence and second amino acid sequence are contiguous and in a sequential order.

B. An isolated polypeptide encoding for an edge portion of D12115_P32 (SEQ. ID NO:141), comprising an amino acid sequence being at least 70%, optionally at least about 80%, preferably at least about 85%, more preferably at least about 90% and most preferably at least about 95% homologous to the sequence GREIQGNKEHNS (SEQ. ID NO: 341) of D12115_P32 (SEQ. ID NO:141).

### 2. Comparison Report Between D12115_P32 (SEQ. ID NO:141) and P00751-2 (SEQ. ID NO:132):

A. An isolated chimeric polypeptide encoding for D12115_P32 (SEQ. ID NO:141), comprising a first amino acid sequence being at least 90% homologous to MGSNLSPQLCLMWILGLLSGGVTTTPWSLARPQGSCSLEGVEIKGGSFRLLQEGQALEYVCPS GFYPYPVQTRTCRSTGSWSTLKTQDQKTVRKAECRAIHCPRPHDFENGEYWPRSPYYNVSDE ISFHCYDGYTLRGSANRTCQVNGRWSGQTAICDNGAGYCSNPGIPIGTRKVGSQYRLEDSVT YHCSROLTLRGSQRRTCQEGGSWSGTEPSCQDSFMYDTPQEVAEAFLSSLTETIEGVDAEDGH GPGEQQKRKIVLDPSGSMNTYLVLDGSDSIGASNFTGAKKCLVNLIEKVASYGVKPRYGLVTY ATYPKIWVKVSEADSSNADWVTKQLNEINYEDHKLKSGTNTKKALQAVYSMMSWPDDVPP EGWNRTRHVIILMTDGLHNMGGDPITVIDEIRDLLYIGKDRKNPREDYLDVYVFGVGPLVNQ VNINALASKKDNEQHVFKVKDMENLEDVFYQMI corresponding to amino acids 1-469 of P00751-2 (SEQ. ID NO:132), which also corresponds to amino acids 1-469 of D12115_P32 (SEQ. ID NO:141), and a second amino acid sequence being at least 70%, optionally at least 80%, preferably at least 85%, more preferably at least 90% and most preferably at least 95% homologous to a polypeptide having the sequence GREIQGNKEHNS (SEQ. ID NO: 341) corresponding to amino acids 470-481 of D12115_P32 (SEQ. ID NO:141), wherein said first amino acid sequence and second amino acid sequence are contiguous and in a sequential order.

B. An isolated polypeptide encoding for an edge portion of D12115_P32 (SEQ. ID NO:141), comprising an amino acid sequence being at least 70%, optionally at least about 80%, preferably at least about 85%, more preferably at least about 90% and most preferably at least about 95% homologous to the sequence GREIQGNKEHNS (SEQ. ID NO: 341) of D12115_P32 (SEQ. ID NO:141).

### 3. Comparison Report Between D12115_P32 (SEQ. 1:13 NO:141) and NP_{—}001701 (SEQ. ID NO:133)

A. An isolated chimeric polypeptide encoding for D12115_P32 (SEQ. ID NO:141), comprising a first amino acid sequence being at least 90% homologous to MGSNLSPQLCLMPFILGLLSGGVTTTPWSLA corresponding to amino acids 1-31 of NP_{—}001701 (SEQ. ID NO:133), which also corresponds to amino acids 1-31 of D12115_P32 (SEQ. ID NO:141), a bridging amino acid R corresponding to amino acid 32 of D12115_P32 (SEQ. ID NO:141), a second amino acid sequence being at least 90% homologous to PQGSCSLEGVETKGGSFRLLQEGQALEYVCPSGFYPYPVQTRTCRSTGSWSTLKTQDQKTVRK AECRAIHCPRPHDFENGEYWPRSPYYNVSDEISFHCYDGYTLRGSANRTCQVNGRWSGQTAI CDNGAGYCSNPGIPIGTRKVGSQYRLEDSVTYHCSRGLTLRGSQRRTCQEGGSWSGTEPSCQ DSFMYDTPQEVAEAFLSSLTETIEGVDAEDGHGPGEQQKRKIVLDPSGSMNIYLVLDGSDSIG ASNFTGAKKCLVNLIEKVASYGVKPRYGLVTYATYPKIWVKVSEADSSNADWVTKQLNEIN YEDHKLKSGTNTKKALQAVYSMMSWPDDVPPEGWNRTRHVIILMTDGLHNMGGDPITVIDE IRDLLYIGKDRKNPREDYLDVYVFGVGPLVNQVNINALASKKDNEQHVFKVKDMENLEDVF YQMI corresponding to amino acids 33-469 of NP_{—}001701 (SEQ. ID NO:133), which also corresponds to amino acids 33-469 of D12115_P32 (SEQ. ID NO:141), and a third amino acid sequence being at least 70%, optionally at least 80%, preferably at least 85%, more preferably at least 90% and most preferably at least 95% homologous to a polypeptide having the sequence GREIQGNKEHNS (SEQ. ID NO: 341) corresponding to amino acids 470-481 of D12115_P32 (SEQ. ID NO:141), wherein said first amino acid sequence, bridging amino acid, second amino acid sequence and third amino acid sequence are contiguous and in a sequential order.

B. An isolated polypeptide encoding for an edge portion of D12115_P32 (SEQ. ID NO:141), comprising an amino acid sequence being at least 70%, optionally at least about 80%, preferably at least about 85%, more preferably at least about 90% and most preferably at least about 95% homologous to the sequence GREIQGNKEHNS (SEQ. ID NO: 341) of D12115_P32 (SEQ. ID NO:141).

The localization of the variant protein was determined according to results from a number of different software programs and analyses, including analyses from SignalP and other specialized programs. The variant protein is believed to be secreted.

Variant protein D12115_P32 (SEQ. ID NO:141) also has the following non-silent SNPs (Single Nucleotide Polymorphisms) as listed in Table 58, (given according to their position(s) on the amino acid sequence, with the alternative amino acid(s) listed.

TABLE 58
Amino acid mutations

| SNP position(s) on amino acid | |
|---|---|
| sequence | Alternative amino acid(s) |
| 9 | L -> H |
| 32 | R -> Q |
| 32 | R -> W |
| 118 | S -> A |
| 142 | N -> I |
| 225 | D -> |
| 225 | D -> E |
| 252 | G -> S |
| 254 | G -> |
| 365 | M -> I |
| 428 | F -> |
| 472 | E -> A |

The glycosylation sites of variant protein D12115_P32 (SEQ. ID NO:141), as compared to the known protein Complement factor B precursor (SEQ. ID NO:131), are described in Table 59 (given according to their position(s) on the amino acid sequence in the first column; the second column indicates whether the glycosylation site is present in the variant protein; and the last column indicates whether the position is different on the variant protein).

TABLE 59
Glycosylation site(s)

| Position(s) on known amino | Present in | Position(s) on |
|---|---|---|
| acid sequence | variant protein? | variant protein |
| 122 | Yes | 122 |
| 142 | Yes | 142 |
| 285 | Yes | 285 |
| 291 | Yes | 291 |
| 378 | Yes | 378 |

The variant protein has the following domains, as determined by using InterPro. The domains are described in Table 60:

TABLE 60
InterPro domain(s)

| Domain description | Analysis type | Position(s) on protein |
|---|---|---|
| von Willebrand factor, type A | FPrintScan | 269-286, 308-322, |
| | | 383-391 |
| Sushi | HMMPfam | 37-86, 103-158, 165-218 |
| von Willebrand factor, type A | HMMPfam | 270-468 |
| Sushi | HMMSmart | 37-89, 103-158, 165-218 |
| von Willebrand factor, type A | HMMSmart | 268-473 |
| von Willebrand factor, type A | ProfileScan | 270-469 |

Variant protein D12115_P32 (SEQ. ID NO:141) is encoded by the following transcript(s): D12115_T33 (SEQ. ID NO:87), for which the coding portion starts at position 514 and ends at position 1956. The transcript also has the following SNPs as listed in Table 61 (given according to their position on the nucleotide sequence, with the alternative nucleic acid listed.

TABLE 61
Nucleic acid SNPs

| SNP position(s) on nucleotide | |
|---|---|
| sequence | Alternative nucleic acid(s) |
| 325 | G -> |
| 539 | T -> A |
| 607 | C -> T |
| 608 | G -> A |
| 865 | T -> G |
| 918 | C -> T |
| 938 | A -> T |
| 963 | G -> A |
| 1017 | G -> A |
| 1185 | C -> T |
| 1188 | C -> A |
| 1188 | C -> |
| 1267 | G -> A |
| 1273 | G -> |
| 1608 | G -> T |
| 1795 | T -> |
| 1878 | C -> T |
| 1894 | C -> T |
| 1928 | A -> C |

Variant protein D12115_P34 (SEQ. ID NO:142) according to the present invention is encoded by transcript D12115_T14 (SEQ. ID NO:83).

The localization of the variant protein was determined according to results from a number of different software programs and analyses, including analyses from SignalP and other specialized programs. The variant protein is believed to be secreted.

Variant protein D12115_P34 (SEQ. ID NO:142) also has the following non-silent SNPs (Single Nucleotide Polymorphisms) as listed in Table 62, (given according to their position(s) on the amino acid sequence, with the alternative amino acid(s) listed.

TABLE 62
Amino acid mutations

| SNP position(s) on amino acid | |
|---|---|
| sequence | Alternative amino acid(s) |
| 9 | L -> H |
| 32 | R -> Q |
| 32 | R -> W |
| 118 | S -> A |
| 142 | N -> I |
| 220 | R -> Q |
| 276 | T -> M |
| 277 | T -> |
| 277 | T -> K |

The glycosylation sites of variant protein D12115_P34 (SEQ. ID NO:142), as compared to the known protein Complement factor B precursor (SEQ. ID NO:131), are described in Table 63 (given according to their position(s) on the amino acid sequence in the first column; the second column indicates whether the glycosylation site is present in the variant protein; and the last column indicates whether the position is different on the variant protein).

TABLE 63
Glycosylation site(s)

| Position(s) on known amino | Present in | Position(s) on |
|---|---|---|
| acid sequence | variant protein? | variant protein |
| 122 | Yes | 122 |
| 142 | Yes | 142 |
| 285 | No | |
| 291 | No | |
| 378 | No | |

The variant protein has the following domains, as determined by using InterPro. The domains are described in Table 64:

TABLE 64
InterPro domain(s)

| Domain description | Analysis type | Position(s) on protein |
|---|---|---|
| Sushi | HMMPfam | 37-86, 103-158 |
| Sushi | HMMSmart | 37-89, 103-158 |

Variant protein D12115_P34 (SEQ. ID NO:142) is encoded by the following transcript(s): D12115_T14 (SEQ. ID NO:83), for which the coding portion starts at position 514 and ends at position 1380. The transcript also has the following SNPs as listed in Table 65 (given according to their position on the nucleotide sequence, with the alternative nucleic acid listed.

TABLE 65
Nucleic acid SNPs

| SNP position(s) on nucleotide | |
|---|---|
| sequence | Alternative nucleic acid(s) |
| 325 | G -> |
| 539 | T -> A |
| 607 | C -> T |
| 608 | G -> A |
| 865 | T -> G |
| 918 | C -> T |
| 938 | A -> T |
| 963 | G -> A |
| 1172 | G -> A |
| 1340 | C -> T |
| 1343 | C -> A |
| 1343 | C -> |
| 1422 | G -> A |
| 1428 | G -> |
| 1653 | C -> T |
| 1876 | G -> T |
| 2063 | T -> |
| 2146 | C -> T |
| 2162 | C -> T |
| 2449 | C -> A |
| 2452 | C -> T |
| 2474 | A -> G |
| 2573 | C -> G |
| 2573 | C -> T |
| 2588 | A -> |
| 2707 | G -> A |
| 2734 | T -> G |
| 2811 | C -> |
| 2859 | A -> |
| 2887 | C -> T |
| 2967 | A -> G |
| 3031 | C -> |
| 3050 | -> C |
| 3099 | C -> T |

Variant protein D12115_P35 (SEQ. ID NO:143) according to the present invention is encoded by transcript D12115_T9 (SEQ. ID NO:80).

The localization of the variant protein was determined according to results from a number of different software programs and analyses, including analyses from SignalP and other specialized programs. The variant protein is believed to be secreted.

Variant protein D12115_P35 (SEQ. ID NO:143) also has the following non-silent SNPs (Single Nucleotide Polymorphisms) as listed in Table 66, (given according to their position(s) on the amino acid sequence, with the alternative amino acid(s) listed.

TABLE 66
Amino acid mutations

| SNP position(s) on amino acid | |
|---|---|
| sequence | Alternative amino acid(s) |
| 9 | L -> H |
| 32 | R -> Q |
| 32 | R -> W |

The glycosylation sites of variant protein D12115_P35 (SEQ. ID NO:143), as compared to the known protein Complement factor B precursor (SEQ. ID NO:131), are described in Table 67 (given according to their position(s) on the amino acid sequence in the first column; the second column indicates whether the glycosylation site is present in the variant protein; and the last column indicates whether the position is different on the variant protein).

TABLE 67
Glycosylation site(s)

| Position(s) on known amino | Present in | Position(s) on |
|---|---|---|
| acid sequence | variant protein? | variant protein |
| 122 | No | |
| 142 | No | |
| 285 | No | |
| 291 | No | |
| 378 | No | |

The variant protein has the following domains, as determined by using InterPro. The domains are described in Table 68:

TABLE 68
InterPro domain(s)

| Domain description | Analysis type | Position(s) on protein |
|---|---|---|
| Sushi | HMMPfam | 37-86 |

Variant protein D12115_P35 (SEQ. ID NO:143) is encoded by the following transcript(s): D12115_T9 (SEQ. ID NO:80), for which the coding portion starts at position 514 and ends at position 879. The transcript also has the following SNPs as listed in Table 69 (given according to their position on the nucleotide sequence, with the alternative nucleic acid listed.

TABLE 69
Nucleic acid SNPs

| SNP position(s) on nucleotide | |
|---|---|
| sequence | Alternative nucleic acid(s) |
| 325 | G -> |
| 539 | T -> A |
| 607 | C -> T |
| 608 | G -> A |
| 1097 | T -> A |
| 1101 | C -> T |
| 1265 | T -> G |
| 1318 | C -> T |
| 1338 | A -> T |
| 1363 | G -> A |
| 1417 | G -> A |
| 1585 | C -> T |
| 1588 | C -> A |
| 1588 | C -> |
| 1667 | G -> A |
| 1673 | G -> |
| 2008 | G -> T |
| 2195 | T -> |
| 2278 | C -> T |
| 2294 | C -> T |
| 2581 | C -> A |
| 2584 | C -> T |
| 2606 | A -> G |
| 2705 | C -> G |
| 2705 | C -> T |
| 2720 | A -> |
| 2839 | G -> A |
| 2866 | T -> G |
| 2943 | C -> |
| 2991 | A -> |
| 3019 | C -> T |
| 3099 | A -> G |
| 3163 | C -> |
| 3182 | -> C |
| 3231 | C -> T |

As noted above, cluster D12115 features 42 segment(s), which were listed in Table 22 above and for which the sequence(s) are given. These segment(s) are portions of nucleic acid sequence(s) which are described herein separately because they are of particular interest. A description of several segments according to the present invention is now provided.

Segment cluster D12115_N4 (SEQ. ID NO:91) according to the present invention is supported by 11 libraries. The number of libraries was determined as previously described. This segment can be found in the following transcript(s): D12115_T9 (SEQ. ID NO:80). Table 70 below describes the starting and ending position of this segment on each transcript.

TABLE 70
Segment location on transcripts

| | Segment | Segment |
|---|---|---|
| Transcript name | starting position | ending position |
| D12115_T9 | 812 | 1211 |
| (SEQ. ID NO: 80) | | |

Segment cluster D12115_N6 (SEQ. ID NO:93) according to the present invention is supported by 4 libraries. The number of libraries was determined as previously described. This segment can be found in the following transcript(s): D12115_T14 (SEQ. ID NO:83). Table 71 below describes the starting and ending position of this segment on each transcript.

TABLE 71
Segment location on transcripts

| | Segment | Segment |
|---|---|---|
| Transcript name | starting position | ending position |
| D12115_T14 | 998 | 1152 |
| (SEQ. ID NO: 83) | | |

Segment cluster D12115_N27 (SEQ. ID NO:96) according to the present invention is supported by 7 libraries. The number of libraries was determined as previously described. This segment can be found in the following transcript(s): D12145_T36 (SEQ. ID NO:88) and D12115_T5 (SEQ. ID NO:79). Table 72 below describes the starting and ending position of this segment on each transcript.

TABLE 72
Segment location on transcripts

| | Segment | Segment |
|---|---|---|
| Transcript name | starting position | ending position |
| D12115_T36 (SEQ. ID NO: 88) | 1682 | 1962 |
| D12115_T5 (SEQ. ID NO: 79) | 1682 | 1962 |

Segment cluster D12115_N34 (SEQ. ID NO:97) according to the present invention is supported by 13 libraries. The number of libraries was determined as previously described. This segment can be found in the following transcript(s): D12115_T33 (SEQ. ID NO:87) and D12115_T36 (SEQ. ID NO:88). Table 73 below describes the starting and ending position of this segment on each transcript.

TABLE 73
Segment location on transcripts

| | Segment | Segment |
|---|---|---|
| Transcript name | starting position | ending position |
| D12115_T33 (SEQ. ID NO: 87) | 1922 | 1957 |
| D12115_T36 (SEQ. ID NO: 88) | 2203 | 2238 |

Segment cluster D12115_N41 (SEQ. ID NO:98) according to the present invention is supported by 182 libraries. The number of libraries was determined as previously described. This segment can be found in the following transcript(s): D12115_T12 (SEQ. ID NO:81), D12115_T13 (SEQ. ID NO:82), D12115_T14 (SEQ. ID NO:83), D12115_T19 (SEQ. ID NO:84), D12115_T22 (SEQ. ID NO:85), D12115_T3 (SEQ. ID NO:78), D12115_T5 (SEQ. ID NO:79) and D12115_T9 (SEQ. ID NO:80). Table 74 below describes the starting and ending position of this segment on each transcript.

TABLE 74
Segment location on transcripts

| | Segment | Segment |
|---|---|---|
| Transcript name | starting position | ending position |
| D12115_T12 (SEQ. ID NO: 81) | 2138 | 2291 |
| D12115_T13 (SEQ. ID NO: 82) | 2138 | 2291 |
| D12115_T14 (SEQ. ID NO: 83) | 2406 | 2559 |
| D12115_T19 (SEQ. ID NO: 84) | 2138 | 2291 |
| D12115_T22 (SEQ. ID NO: 85) | 2138 | 2291 |
| D12115_T3 (SEQ. ID NO: 78) | 2138 | 2291 |
| D12115_T5 (SEQ. ID NO: 79) | 2419 | 2572 |
| D12115_T9 (SEQ. ID NO: 80) | 2538 | 2691 |

Segment cluster D12115_N53 (SEQ. ID NO:99) according to the present invention is supported by 8 libraries. The number of libraries was determined as previously described. This segment can be found in the following transcript(s): D12115_T12 (SEQ. ID NO:81) and D12115_T22 (SEQ. ID NO:85). Table 75 below describes the starting and ending position of this segment on each transcript.

TABLE 75
Segment location on transcripts

| | Segment | Segment |
|---|---|---|
| Transcript name | starting position | ending position |
| D12115_T12 (SEQ. ID NO: 81) | 2653 | 2922 |
| D12115_T22 (SEQ. ID NO: 85) | 2749 | 3018 |

According to an optional embodiment of the present invention, short segments related to the above cluster are also provided. These segments are up to about 120 by in length, and so are included in a separate description.

Segment cluster D12115_N39 (SEQ. ID NO:119) according to the present invention is supported by 152 libraries. The number of libraries was determined as previously described. This segment can be found in the following transcript(s): D12115_T12 (SEQ. ID NO:81), D12115_T13 (SEQ. ID NO:82), D12115_T14 (SEQ. ID NO:83), D12115_T19 (SEQ. ID NO:84), D12115_T22 (SEQ. ID NO:85), D12115_T27 (SEQ. ID NO:86), D12115_T3 (SEQ. ID NO:78), D12115_T5 (SEQ. ID NO:79) and D12115_T9 (SEQ. ID NO:80). Table 76 below describes the starting and ending position of this segment on each transcript.

TABLE 76
Segment location on transcripts

| | Segment | Segment |
|---|---|---|
| Transcript name | starting position | ending position |
| D12115_T12 (SEQ. ID NO: 81) | 2084 | 2137 |
| D12115_T13 (SEQ. ID NO: 82) | 2084 | 2137 |
| D12115_T14 (SEQ. ID NO: 83) | 2352 | 2405 |
| D12115_T19 (SEQ. ID NO: 84) | 2084 | 2137 |
| D12115_T22 (SEQ. ID NO: 85) | 2084 | 2137 |
| D12115_T27 (SEQ. ID NO: 86) | 2084 | 2137 |
| D12115_T3 (SEQ. ID NO: 78) | 2084 | 2137 |
| D12115_T5 (SEQ. ID NO: 79) | 2365 | 2418 |
| D12115_T9 (SEQ. ID NO: 80) | 2484 | 2537 |

Segment cluster D12115_N43 (SEQ. ID NO:120) according to the present invention is supported by 178 libraries. The number of libraries was determined as previously described. This segment can be found in the following transcript(s): D12115_T12 (SEQ. ID NO:81), D12115_T13 (SEQ. ID NO:82), D12115_T14 (SEQ. ID NO:83), D12115_T22 (SEQ. ID NO:85), D12115_T3 (SEQ. ID NO:78), D12115_T5 (SEQ. ID NO:79) and D12115_T9 (SEQ. ID NO:80). Table 77 below describes the starting and ending position of this segment on each transcript.

TABLE 77
Segment location on transcripts

| | Segment | Segment |
|---|---|---|
| Transcript name | starting position | ending position |
| D12115_T12 (SEQ. ID NO: 81) | 2292 | 2368 |
| D12115_T13 (SEQ. ID NO: 82) | 2292 | 2368 |
| D12115_T14 (SEQ. ID NO: 83) | 2560 | 2636 |
| D12115_T22 (SEQ. ID NO: 85) | 2292 | 2368 |
| D12115_T3 (SEQ. ID NO: 78) | 2292 | 2368 |
| D12115_T5 (SEQ. ID NO: 79) | 2573 | 2649 |
| D12115_T9 (SEQ. ID NO: 80) | 2692 | 2768 |

Segment cluster D12115_N45 (SEQ. ID NO:121) according to the present invention is supported by 171 libraries. The number of libraries was determined as previously described. This segment can be found in the following transcript(s): D12115_T12 (SEQ. ID NO:81), D12115_T14 (SEQ. ID NO:83), D12115_T19 (SEQ. ID NO:84), D12115_T22 (SEQ. ID NO:85), D12115_T27 (SEQ. ID NO:86), D12115_T3 (SEQ. ID NO:78), D12115_T5 (SEQ. ID NO:79) and D12115_T9 (SEQ. ID NO:80). Table 78 below describes the starting and ending position of this segment on each transcript.

TABLE 78
Segment location on transcripts

| | Segment | Segment |
|---|---|---|
| Transcript name | starting position | ending position |
| D12115_T12 (SEQ. ID NO: 81) | 2369 | 2370 |
| D12115_T14 (SEQ. ID NO: 83) | 2637 | 2638 |
| D12115_T19 (SEQ. ID NO: 84) | 2292 | 2293 |
| D12115_T22 (SEQ. ID NO: 85) | 2369 | 2370 |
| D12115_T27 (SEQ. ID NO: 86) | 2138 | 2139 |
| D12115_T3 (SEQ. ID NO: 78) | 2369 | 2370 |
| D12115_T5 (SEQ. ID NO: 79) | 2650 | 2651 |
| D12115_T9 (SEQ. ID NO: 80) | 2769 | 2770 |

Segment cluster D12115_N46 (SEQ. ID NO:122) according to the present invention is supported by 190 libraries. The number of libraries was determined as previously described. This segment can be found in the following transcript(s): D12115_T12 (SEQ. ID NO:81), D12115_T13 (SEQ. ID NO:82), D12115_T14 (SEQ. ID NO:83), D12115_T19 (SEQ. ID NO:84), D12115_T22 (SEQ. ID NO:85), D12115_T27 (SEQ. ID NO:86), D12115_T3 (SEQ. ID NO:78), D12115_T5 (SEQ. ID NO:79) and D12115_T9 (SEQ. ID NO:80). Table 79 below describes the starting and ending position of this segment on each transcript.

TABLE 79
Segment location on transcripts

| | Segment | Segment |
|---|---|---|
| Transcript name | starting position | ending position |
| D12115_T12 (SEQ. ID NO: 81) | 2371 | 2401 |
| D12115_T13 (SEQ. ID NO: 82) | 2369 | 2399 |
| D12115_T14 (SEQ. ID NO: 83) | 2639 | 2669 |
| D12115_T19 (SEQ. ID NO: 84) | 2294 | 2324 |
| D12115_T22 (SEQ. ID NO: 85) | 2371 | 2401 |
| D12115_T27 (SEQ. ID NO: 86) | 2140 | 2170 |
| D12115_T3 (SEQ. ID NO: 78) | 2371 | 2401 |
| D12115_T5 (SEQ. ID NO: 79) | 2652 | 2682 |
| D12115_T9 (SEQ. ID NO: 80) | 2771 | 2801 |

Segment cluster D12115_N47 (SEQ. ID NO:123) according to the present invention is supported by 204 libraries. The number of libraries was determined as previously described. This segment can be found in the following transcript(s): D12115_T12 (SEQ. ID NO:81), D12115_T13 (SEQ. ID NO:82), D12115_T14 (SEQ. ID NO:83), D12115_T19 (SEQ. ID NO:84), D12115_T22 (SEQ. ED NO:85), D12115_T27 (SEQ. ID NO:86), D12115_T3 (SEQ. ID NO:78), D12115_T5 (SEQ. ID NO:79) and D12115_T9 (SEQ. ID NO:80). Table 80 below describes the starting and ending position of this segment on each transcript.

TABLE 80
Segment location on transcripts

| | Segment | Segment |
|---|---|---|
| Transcript name | starting position | ending position |
| D12115_T12 (SEQ. ID NO: 81) | 2402 | 2469 |
| D12115_T13 (SEQ. ID NO: 82) | 24 | 2467 |
| D12115_T14 (SEQ. ID NO: 83) | 2670 | 2737 |
| D12115_T19 (SEQ. ID NO: 84) | 2325 | 2392 |
| D12115_T22 (SEQ. ID NO: 85) | 2402 | 2469 |
| D12115_T27 (SEQ. ID NO: 86) | 2171 | 2238 |
| D12115_T3 (SEQ. ID NO: 78) | 2402 | 2469 |
| D12115_T5 (SEQ. ID NO: 79) | 2683 | 2750 |
| D12115_T9 (SEQ. ID NO: 80) | 2802 | 2869 |

Segment cluster D12115_N48 (SEQ. ID NO:124) according to the present invention is supported by 3 libraries. The number of libraries was determined as previously described. This segment can be found in the following transcript(s): D12115_T22 (SEQ. ID NO:85). Table 81 below describes the starting and ending position of this segment on each transcript.

TABLE 81
Segment location on transcripts

| | Segment | Segment |
|---|---|---|
| Transcript name | starting position | ending position |
| D12115_T22 | 2470 | 2565 |
| (SEQ. ID NO: 85) | | |

Segment cluster D12115_N54 (SEQ. ID NO:128) according to the present invention is supported by 182 libraries. The number of libraries was determined as previously described. This segment can be found in the following transcript(s): D12115_T12 (SEQ. ID NO:81), D12115_T13 (SEQ. ID NO:82), D12115_T14 (SEQ. ID NO:83), D12115_T19 (SEQ. ID NO:84), D12115_T22 (SEQ. ID NO:85), D12115_T27 (SEQ. ID NO:86), D12115_T3 (SEQ. ID NO:78), D12115_T5 (SEQ. ID NO:79) and D12115_T9 (SEQ. ID NO:80). Table 82 below describes the starting and ending position of this segment on each transcript.

TABLE 82
Segment location on transcripts

| | Segment | Segment |
|---|---|---|
| Transcript name | starting position | ending position |
| D12115_T12 (SEQ. ID NO: 81) | 2923 | 2972 |
| D12115_T13 (SEQ. ID NO: 82) | 2651 | 27 |
| D12115_T14 (SEQ. ID NO: 83) | 2921 | 2970 |
| D12115_T19 (SEQ. ID NO: 84) | 2576 | 2625 |
| D12115_T22 (SEQ. ID NO: 85) | 3019 | 3068 |
| D12115_T27 (SEQ. ID NO: 86) | 2422 | 2471 |
| D12115_T3 (SEQ. ID NO: 78) | 2653 | 2702 |
| D12115_T5 (SEQ. ID NO: 79) | 2934 | 2983 |
| D12115_T9 (SEQ. ID NO: 80) | 3053 | 3102 |

Segment cluster D12115_N55 (SEQ. ID NO:129) according to the present invention is supported by 172 libraries. The number of libraries was determined as previously described. This segment can be found in the following transcript(s): D12115_T12 (SEQ. ID NO:81), D12115_T13 (SEQ. ID NO:82), D12115_T14 (SEQ. ID NO:83), D12115_T19 (SEQ. ID NO:84), D12115_T22 (SEQ. ID NO:85), D12115_T27 (SEQ. ID NO:86), D12115_T5 (SEQ. ID NO:79) and D12115_T9 (SEQ. ID NO:80). Table 83 below describes the starting and ending position of this segment on each transcript.

TABLE 83
Segment location on transcripts

| | Segment | Segment |
|---|---|---|
| Transcript name | starting position | ending position |
| D12115_T12 (SEQ. ID NO: 81) | 2973 | 3025 |
| D12115_T13 (SEQ. ID NO: 82) | 2701 | 2753 |
| D12115_T14 (SEQ. ID NO: 83) | 2971 | 3023 |
| D12115_T19 (SEQ. ID NO: 84) | 2626 | 2678 |
| D12115_T22 (SEQ. ID NO: 85) | 3069 | 3121 |
| D12115_T27 (SEQ. ID NO: 86) | 2472 | 2524 |
| D12115_T5 (SEQ. ID NO: 79) | 2984 | 3036 |
| D12115_T9 (SEQ. ID NO: 80) | 3103 | 3155 |

Segment cluster D12115_N56 (SEQ. ID NO:130) according to the present invention is supported by 154 libraries. The number of libraries was determined as previously described. This segment can be found in the following transcript(s): D12115_T12 (SEQ. ID NO:81), D12115_T13 (SEQ. ID NO:82), D12115_T14 (SEQ. ID NO:83), D12115_T19 (SEQ. ID NO:84), D12115_T13 (SEQ. ID NO:85), D12115_T27 (SEQ. ID NO:86), D12115_T3 (SEQ. ID NO:78), D12115_T5 (SEQ. ID NO:79) and D12115_T9 (SEQ. ID NO:80). Table 84 below describes the starting and ending position of this segment on each transcript.

TABLE 84
Segment location on transcripts

| | Segment | Segment |
|---|---|---|
| Transcript name | starting position | ending position |
| D12115_T12 (SEQ. ID NO: 81) | 3026 | 3132 |
| D12115_T13 (SEQ. ID NO: 82) | 2754 | 2860 |
| D12115_T14 (SEQ. ID NO: 83) | 3024 | 3130 |
| D12115_T19 (SEQ. ID NO: 84) | 2679 | 2785 |
| D12115_T22 (SEQ. ID NO: 85) | 3122 | 3228 |
| D12115_T27 (SEQ. ID NO: 86) | 2525 | 2631 |
| D12115_T3 (SEQ. ID NO: 78) | 2703 | 2809 |
| D12115_T5 (SEQ. ID NO: 79) | 3037 | 3143 |
| D12115_T9 (SEQ. ID NO: 80) | 3156 | 3262 |

Expression of Homo sapiens B-Factor, Properdin (BF) D12115 Transcripts Which are Detectable by Junction 0-2 and Segment 6 in Normal and Cancerous Ovary and Breast Tissues

Expression of Homo sapiens B-factor, properdin (BF) transcripts detectable by or according to junction 0-2 and segment 6 was measured with oligonucleotide-based micro-arrays. The results of image intensities for each feature were normalized according to the ninetieth percentile of the image intensities of all the features on the chip. Then, feature image intensities for replicates of the same oligonucleotide on the chip and replicates of the same sample were averaged. Outlying results were discarded.

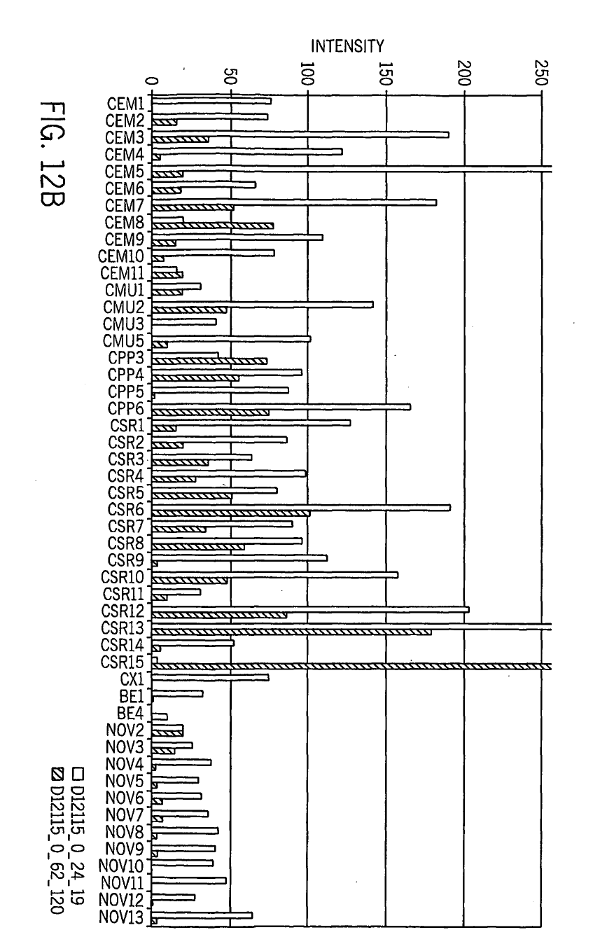

For every oligonucleotide (D12115_{—}0_{—}24_{—}19 (SEQ. ID NO: 324) and D12115_{—}0_{—}62_{—}120 (SEQ. ID NO: 325)) the calculated intensities in the different tissue samples hybridized are presented in figures FIG. 12A for ovary samples and FIG. 12B for breast samples. As is evident from the histogram, the expression of Homo sapiens B-factor, properdin (BF) transcripts detectable with the above oligonucleotides in ovary and breast cancer samples was higher than in the normal samples.

D12115_0_24_19

(SEQ. ID NO: 324)

AGTGGGCACTCGGCTCCGGACACTGTAACTCTTGCTCTCTACCTTGCTCA
D12115_0_62_120

(SEQ. ID NO: 325)

ATGCCCTTTATCTTGGGCCTCTTGTCTGGAGGTGTGACCACCACTCCATG

Expression of Homo sapiens B-factor, properdin (BF) D12115 transcripts, detectable by or according to D12115_seg4 (SEQ. ID NO: 146) amplicon and primers D12115_seg4F (SEQ. ID NO: 144) and D12115_seg4R (SEQ. ID NO: 145); or of Homo sapiens B-factor, properdin (BF) D12115 transcripts, detectable by or according to D12115_seg6 (SEQ. ID NO: 149) amplicon and primers D12115_seg6F (SEQ. ID NO: 147) and D12115_seg6R (SEQ. ID NO: 148); or Homo sapiens B-factor, properdin (BF) transcripts detectable by or according to seg40WT—D12115_seg40WT (SEQ. ID NO: 152) amplicon and primers D12115_seg40WTF (SEQ. ID NO: 150) and D12115_seg40WTR (SEQ. ID NO: 151); or Homo sapiens B-factor, properdin (BF) transcripts detectable by or according to seg46-47—D12115_seg46-47 (SEQ. ID NO:. 155) amplicon and primers D12115_seg46-47F (SEQ. ID NO: 153) and D12115_seg46-47R (SEQ. ID NO: 154); or Homo sapiens B-factor, properdin (BF) transcripts detectable by or according to seg27 and seg34—D12115seg27 (SEQ. ID NO: 320) and D12115seg34 (SEQ. ID NO: 323) amplicons and primers D12115seg27F (SEQ. ID NO: 318), D12115seg27R (SEQ. ID NO: 319), D12115seg34F (SEQ. ID NO: 321) and D12115seg34R (SEQ. ID NO: 322) was measured by real time PCR.

The sequences of corresponding primers and amplicons are given below.

Forward Primer D12115_seg4F (SEQ. ID NO: 144)
(SEQ. ID NO: 144): GTTTGAGGGCAATGAGTGTGG
Reverse Primer D12115_seg4R
(SEQ. ID NO: 145): AAACTGCTCCTACTCCCGGTC
Amplicon D12115_seg4
(SEQ. ID NO: 146): GTTTGAGGGCAATGAGTGTGGGCAGTGGCCT
AAGGCAGAAACAGGGCAGGCGGCAGCAAGGTCAGGACTAGGATGAGACTA
GGCAGGGTGACAAGGTGGGCTGACCGGGAGTAGGAGCAGTTT
Forward Primer D12115_seg6F
(SEQ. ID NO: 147): CTACATTGCTGTCTCCCTGACG
Reverse Primer D12115_seg6R
(SEQ. ID NO: 148): AGGTAAGCACTGAAGCCTGAGG
Amplicon D12115_seg6
(SEQ. ID NO: 149): CTACATTGCTGTCTCCCTGACGGCGCCCAGC
CCGAGGAGTGGGCACTCGGCTCCGGACACTGTAACTCTTGCTCT
CTACCTTGCTCACGGGGCCTCAGGCTTCAGTGCTTACCT
Forward Primer D12115_seg40WTF
(SEQ. ID NO: 150): AGGCAACACCTCCCACTTTCT
Reverse Primer D12115_seg40WTR
(SEQ. ID NO: 151): TTCACGTCTTCCCCCATCC
Amplicon D12115_seg40WT
(SEQ. ID NO: 152): AGGCAACACCTCCCACTTTCTACAGATCCTA
CACTCCACCCATCCTCAATGCAGCCCCATTCCTTGCACCCCAGACC
AGTCAGGGATGGGGGAAGACGTGA
Forward Primer D12115_seg46-47F
(SEQ. ID NO: 153): GAAGAGCTGCTCCCTGCA
Reverse Primer D12115_seg46-47R
(SEQ. ID NO: 154): CCCCATTCTTGATGTAGACCTC
Amplicon (D12115_seg46-47
(SEQ. ID NO: 155): GAAGAGCTGCTCCCTGCACAGGATATCAAAG
CTCTGTTTGTGTCTGAGGAGGAGAAAAAGCTGACTCGGAAGGAGGTCT
ACATCAAGAATGGGG
Forward Primer D12115seg27F
(SEQ. ID NO: 318): TGTCCCAGCCTCCCCAC
Reverse Primer D12115seg27R
(SEQ. ID NO: 319): GAGTCACATTCAGGGCCCC
Amplicon D12115seg27
(SEQ. ID NO: 320): TGTCCCAGCCTCCCCACCTTCTCAGACCAGC
ATGTGGCCCTTAAGTCCACTTGTAACACTATACCCATGGTTGGGGCCCTG
AATGTGACTC
Forward Primer D12115seg34F
(SEQ. ID NO: 321): CAACTCTCCTCAGGTTCCCCT
Reverse Primer D12115seg34R
(SEQ. ID NO: 322): GAGAAGGAGGAATGAAGAAGGCTT
Amplicon D12115seg34
(SEQ. ID NO: 323): CAACTCTCCTCAGGTTCCCCTGAAGTAATTC
ATTCTTCCTCTACACCTGAAGCTCTAGTTGCCTGGAAAGCCTTC
TTCATTCCTCCTTCTC

Expression of Homo sapiens B-factor, properdin (BF) D12115 transcripts which are detectable by amplicon as depicted in sequence name D12115_seg4 (SEQ. ID NO: 146) in normal and cancerous Breast tissues

Expression of Homo sapiens B-factor, properdin (BF) transcripts detectable by or according to seg4-D12115_seg4 (SEQ. ID NO: 146) amplicon and primers D12115_seg4F (SEQ. ID NO: 144) and D12115_seg4R (SEQ. ID NO: 145) was measured by real time PCR. In parallel the expression of four housekeeping genes—G6PD (GenBank Accession No. NM_{—}000402 (SEQ. ID NO: 13); G6PD amplicon (SEQ. ID NO: 16)), HPRT1 (GenBank Accession No. NM_{—}000194 (SEQ. ID NO: 5); amplicon—HPRT1-amplicon (SEQ. ID NO: 8)), PBGD (GenBank Accession No. BC019323 (SEQ. ID NO: 1); amplicon—PBGD-amplicon (SEQ. ID NO: 4)) and SDHA (GenBank Accession No. NM_{—}004168 (SEQ. ID NO: 33); amplicon—SDHA-amplicon (SEQ. ID NO:36)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above), to obtain a value of fold up-regulation for each sample relative to median of the normal PM samples.

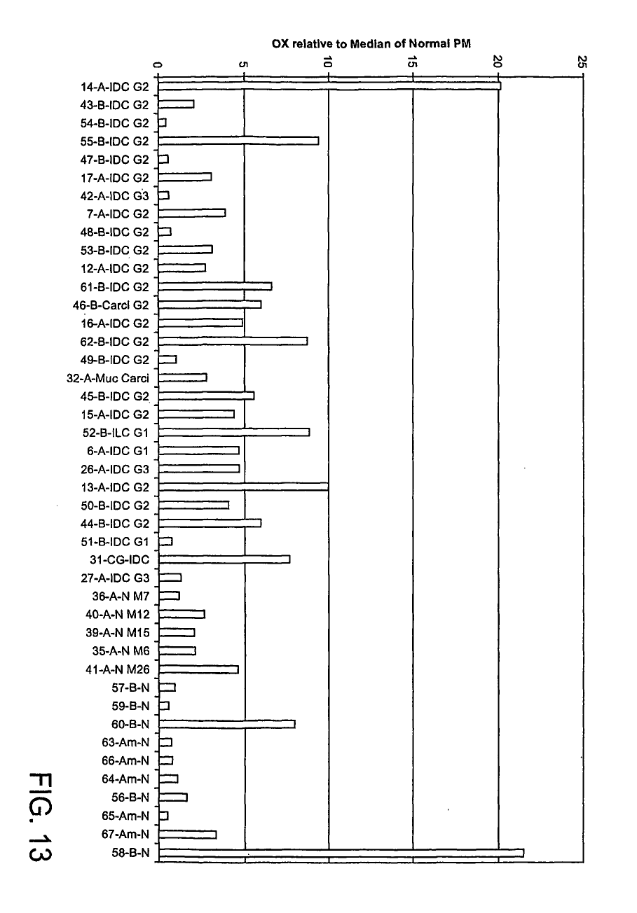

FIG. 13 is a histogram showing over expression of the above-indicated Homo sapiens B-factor, properdin (BF) transcripts in cancerous Breast samples relative to the normal samples.

As is evident from FIG. 13, the expression of Homo sapiens B-factor, properdin (BF) transcripts detectable by the above amplicon in cancer samples was higher than in the non-cancerous samples (sample numbers 57, 59, 60, 63, 66, 64, 56, 65, 67 and 58, Table 1_{—}3 above). Notably an over-expression of at least 5 fold was found in 10 out of 28 adenocarcinoma samples.

Primer pairs are also optionally and preferably encompassed within the present invention; for example, for the above experiment, the following primer pair was used as a non-limiting illustrative example only of a suitable primer pair: D12115_seg4F (SEQ. ID NO: 144) forward primer; and D12115_seg4R (SEQ. ID NO: 145) reverse primer.

The present invention also preferably encompasses any amplicon obtained through the use of any suitable primer pair; for example, for the above experiment, the following amplicon was obtained as a non-limiting illustrative example only of a suitable amplicon: D12115_seg4 (SEQ. ID NO: 146).

Expression of Homo sapiens B-factor, properdin (BF) D12115 transcripts which are detectable by amplicon as depicted in sequence name D12115_seg4 (SEQ. ID NO: 146) in different normal tissues

Expression of Homo sapiens B-factor, properdin (BF) transcripts detectable by or according to seg4-D12115_seg4 (SEQ. ID NO: 146) amplicon and primers D12115_seg4F (SEQ. ID NO: 144) and D12115_seg4R (SEQ. ID NO: 145) was measured by real time PCR. In parallel the expression of four housekeeping genes—SDHA (GenBank Accession No. NM004168 (SEQ. ID NO: 33); amplicon—SDHA-amplicon (SEQ. ID NO:36)), Ubiquitin (GenBank Accession No. BC000449 (SEQ. ID NO: 29); amplicon—Ubiquitin-amplicon (SEQ. ID NO: 32)), RPL19 (GenBank Accession No. NM_{—}000981 (SEQ. ID NO: 21); RPL19 amplicon (SEQ. ID NO: 24)) and TATA box (GenBank Accession No. NM_{—}003194 (SEQ. ID NO: 25); TATA amplicon (SEQ. ID NO: 28)) was measured similarly. For each RT sample, the expression of the above amplicon was normalized to the geometric mean of the quantities of the housekeeping genes. The normalized quantity of each RT sample was then divided by the median of the quantities of the ovary samples (sample numbers 19 and 20, Table 1_{—}6 above), to obtain a value of relative expression of each sample relative to median of the ovary samples.

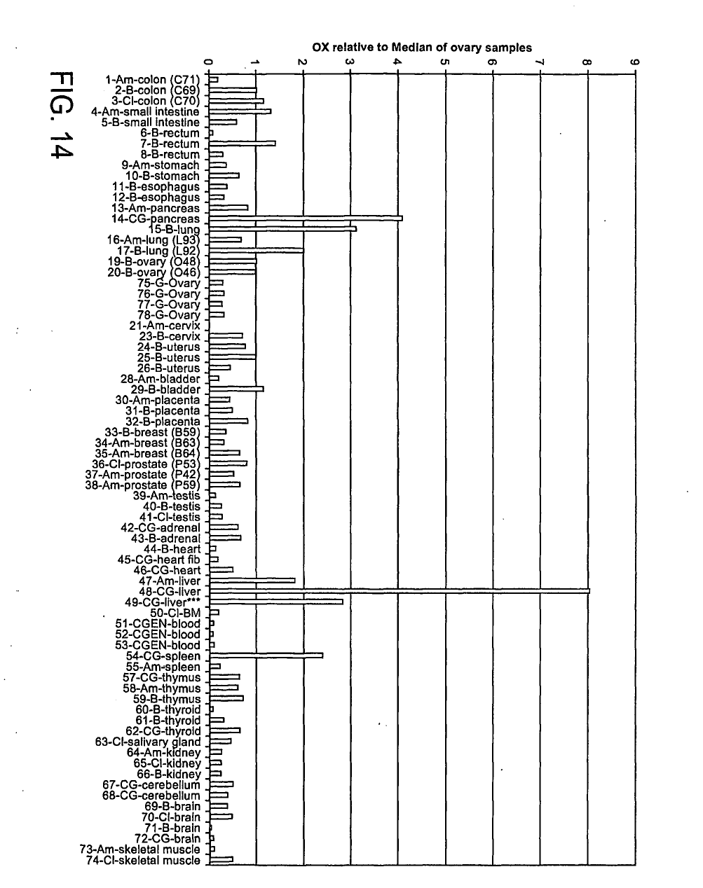

FIG. 14 is a histogram showing expression of the Homo sapiens B-factor, properdin (BF) D12115 transcripts which are detectable by amplicon as depicted in sequence name D12115_seg4 (SEQ. ID NO: 146) in different normal samples.

Expression of Homo sapiens B-factor, properdin (BF) D12115 transcripts which are detectable by amplicon as depicted in sequence name D12115seg4 (SEQ. ID NO: 146) in normal and cancerous Ovary tissues

Expression of Homo sapiens B-factor, properdin (BF) transcripts detectable by or according to seg4-D12115_seg4 (SEQ. ID NO: 146) amplicon and primers D12115_seg4F (SEQ. 113 NO: 144) and D12115_seg4R (SEQ. ID NO: 145) was measured by real time PCR. In parallel the expression of four housekeeping genes—SDHA (GenBank Accession No. NM_{—}004168 (SEQ. ID NO: 33); amplicon—SDHA-amplicon (SEQ. ID NO:36)), HPRT1 (GenBank Accession No. NM_{—}000194 (SEQ. ID NO: 5); amplicon—HPRT1-amplicon (SEQ. ID NO: 8)), PBGD (GenBank Accession No. BC019323 (SEQ. ID NO: 1); amplicon—PBGD-amplicon (SEQ. ID NO: 4)) and GAPDH (GenBank Accession No. BC026907 (SEQ. ID NO: 9); GAPDH amplicon (SEQ. ID NO: 12)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to median of the normal PM samples.

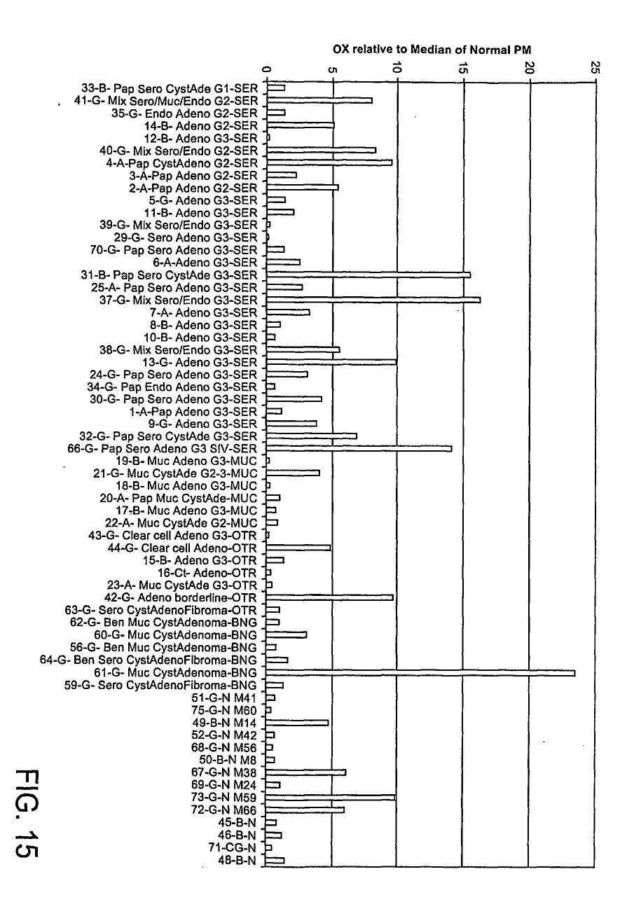

FIG. 15 is a histogram showing over expression of the above-indicated Homo sapiens B-factor, properdin (BF) transcripts in cancerous Ovary samples relative to the normal samples.

As is evident from FIG. 15, the expression of Homo sapiens B-factor, properdin (BF) transcripts detectable by the above amplicon in serous carcinoma samples was higher than in the non-cancerous samples (sample numbers 45, 46, 71 and 48, Table 1_{—}1 above). Notably an over-expression of at least 5 fold was found in 12 out of 43 adenocarcinoma samples, specifically in 11 out of 30 serous carcinoma samples.

Statistical analysis was applied to verify the significance of these results, as described below.

The P value for the difference in the expression levels of Homo sapiens B-factor, properdin (BF) transcripts detectable by the above amplicon in Ovary serous carcinoma samples versus the normal tissue samples was determined by T test as 2.19e-04. The P value for the difference in the expression levels of Homo sapiens B-factor, properdin (BF) transcripts detectable by the above amplicon in Ovary adenocarcinoma samples versus the normal tissue samples was determined by T test as 1.75e-04.

The above values demonstrate statistical significance of the results.

Primer pairs are also optionally and preferably encompassed within the present invention; for example, for the above experiment, the following primer pair was used as a non-limiting illustrative example only of a suitable primer pair: D12115_seg4F (SEQ. ID NO: 144) forward primer; and D12115_seg4R (SEQ. ID NO: 145) reverse primer.

The present invention also preferably encompasses any amplicon obtained through the use of any suitable primer pair; for example, for the above experiment, the following amplicon was obtained as a non-limiting illustrative example only of a suitable amplicon: D12115_seg4 (SEQ. ID NO: 146).

Expression of Homo sapiens B-factor, properdin (BF) D12115 transcripts which are detectable by amplicon as depicted in sequence name D12115_seg6 (SEQ. ID NO: 149) in normal and cancerous Breast tissues

Expression of Homo sapiens B-factor, properdin (BF) transcripts detectable by or according to seg6-D12115_seg6 (SEQ. ID NO: 149) amplicon and primers D12115_seg6F (SEQ. ID NO: 147) and D12115_seg6R (SEQ. ID NO: 148) was measured by real time PCR. In parallel the expression of four housekeeping genes—G6PD (GenBank Accession No. NM_{—}000402 (SEQ. ID NO: 13); G6PD amplicon (SEQ. ID NO: 16)), HPRT1 (GenBank Accession No. NM_{—}000194 (SEQ. ID NO: 5); amplicon—HPRT1-amplicon (SEQ. ID NO: 8)), PBGD (GenBank Accession No. BC019323 (SEQ. ID NO: 1); amplicon—PBGD-amplicon (SEQ. ID NO: 4)) and SDHA (GenBank Accession No. NM_{—}004168 (SEQ. ID NO: 33); amplicon—SDHA-amplicon (SEQ. ID NO:36)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 57, 59, 60, 63, 66, 64, 56, 65, 67 and 58, Table 1_{—}3 above), to obtain a value of fold up-regulation for each sample relative to median of the normal PM samples.

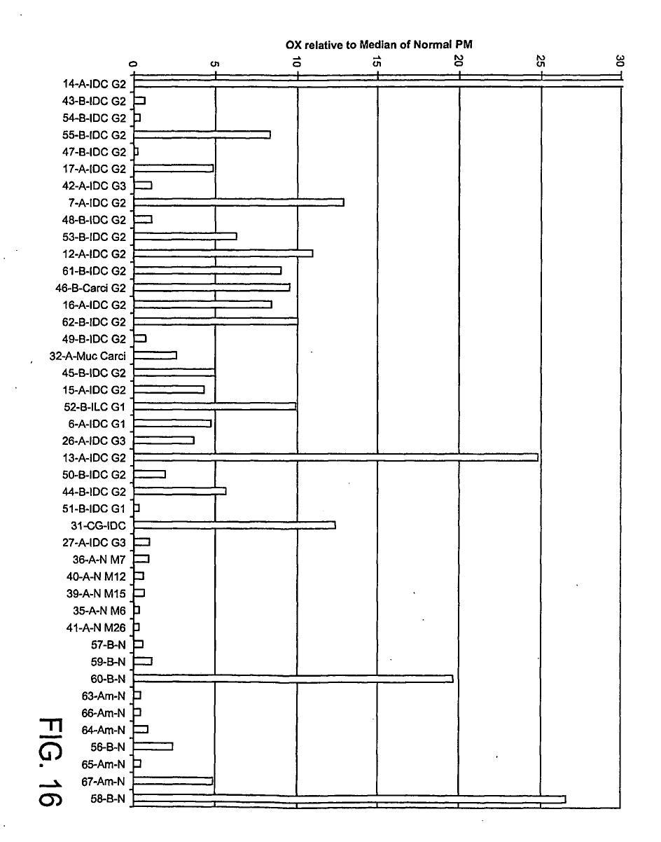

FIG. 16 is a histogram showing over expression of the above-indicated Homo sapiens B-factor, properdin (BF) transcripts in cancerous Breast samples relative to the normal samples.

As is evident from FIG. 16, the expression of Homo sapiens B-factor, properdin (BF) transcripts detectable by the above amplicon in cancer samples was higher than in the non-cancerous samples (sample numbers 57, 59, 60, 63, 66, 64, 56, 65, 67 and 58, Table 1_{—}3 above). Notably an over-expression of at least 5 fold was found in 14 out of 28 adenocarcinoma samples.

Primer pairs are also optionally and preferably encompassed within the present invention; for example, for the above experiment, the following primer pair was used as a non-limiting illustrative example only of a suitable primer pair: D12115_seg6F (SEQ. ID NO: 147) forward primer; and D12115seg6R (SEQ. ID NO: 148) reverse primer.

The present invention also preferably encompasses any amplicon obtained through the use of any suitable primer pair; for example, for the above experiment, the following amplicon was obtained as a non-limiting illustrative example only of a suitable amplicon: D12115_seg6 (SEQ. ID NO: 149).

Expression of Homo sapiens B-factor, properdin (BF) D12115 transcripts which are detectable by amplicon as depicted in sequence name D12115_seg6 (SEQ. ID NO: 149) in normal and cancerous Lung tissues

Expression of Homo sapiens B-factor, properdin (BF) transcripts detectable by or according to seg6-D12115_seg6 (SEQ. ID NO: 149) amplicon and primers D12115_seg6F (SEQ. ID NO: 147) and D12115_seg6R (SEQ. ID NO: 148) was measured by real time PCR. In parallel the expression of four housekeeping genes—HPRT1 (GenBank Accession No. NM_{—}000194 (SEQ. ID NO: 5); amplicon—HPRT1-amplicon (SEQ. ID NO: 8)), PBGD (GenBank Accession No. BC019323 (SEQ. ID NO: 1); amplicon—PBGD-amplicon (SEQ. ID NO: 4)), SDHA (GenBank Accession No. M4004168 (SEQ. ID NO: 33); amplicon—SDHA-amplicon (SEQ. ID NO:36)) and Ubiquitin (GenBank Accession No. BC000449 (SEQ. ID NO: amplicon—Ubiquitin-amplicon (SEQ. ID NO: 32)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 47, 48, 49, 50, 90, 91, 92, 93, 96, 97, 98 and 99, Table 1_{—}2 above), to obtain a value of fold up-regulation for each sample relative to median of the normal PM samples.

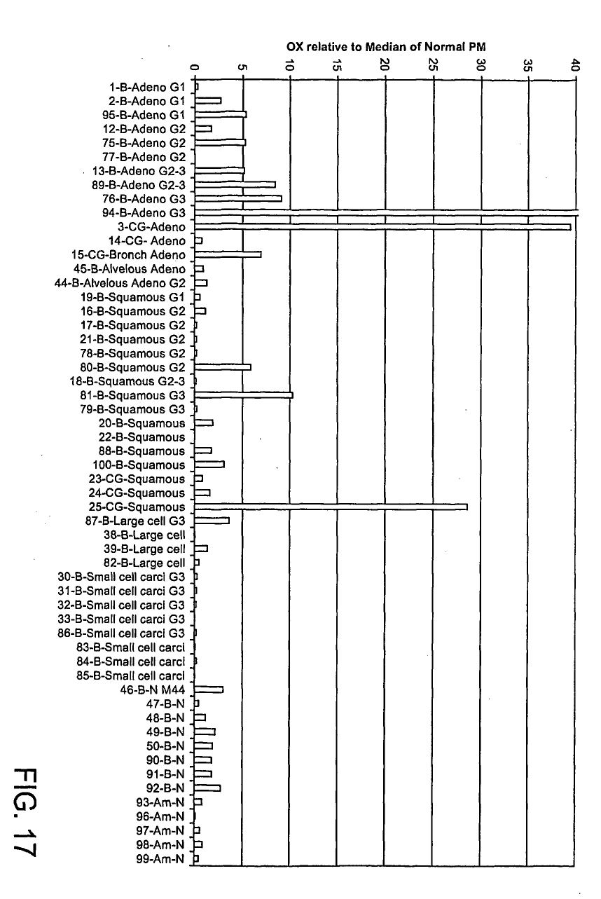

FIG. 17 is a histogram showing over expression of the above-indicated Homo sapiens B-factor, properdin (BF) transcripts in cancerous Lung samples relative to the normal samples.

As is evident from FIG. 17, the expression of Homo sapiens B-factor, properdin (BF) transcripts detectable by the above amplicon in adenocarcinoma and squamous cell carcinoma was higher than in the non-cancerous samples (sample numbers 47, 48, 49, 50, 90, 91, 92, 93, 96, 97, 98 and 99, Table 1_{—}2 above). Notably an over-expression of at least 5 fold was found in 8 out of 15 adenocarcinoma samples and in 3 out of 16 squamous cell carcinoma.

Statistical analysis was applied to verify the significance of these results, as described below.

The P value for the difference in the expression levels of Homo sapiens B-factor, properdin (BF) transcripts detectable by the above amplicon in Lung non-small cell carcinoma samples versus the normal tissue samples was determined by T test as 2.64e-02.

Threshold of 5 fold over expression was found to differentiate between adenocarcinoma and normal samples with P value of 2.90e-03 as checked by exact Fisher test. Threshold of 5 fold over expression was found to differentiate between non-small cell carcinoma and normal samples with P value of 2.40e-02 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: D12115_seg6F (SEQ. ID NO: 147) forward primer; and D12115seg6R (SEQ. ID NO: 148) reverse primer.

The present invention also preferably encompasses any amplicon obtained through the use of any suitable primer pair; for example, for the above experiment, the following amplicon was obtained as a non-limiting illustrative example only of a suitable amplicon: D12115_seg6 (SEQ. ID NO: 149).

Expression of Homo sapiens B-Factor, Properdin (BF) D12115 Transcripts Which are Detectable by Amplicon as Depicted in Sequence Name D12115 Seg6 (SEQ. ID NO: 149) in Different Normal Tissues

Expression of Homo sapiens B-factor, properdin (BF) transcripts detectable by or according to seg6-D12115_seg6 (SEQ. ID NO: 149) amplicon and primers D12115_seg6F (SEQ. ID NO: 147and D12115_seg6R (SEQ. ID NO: 148) was measured by real time PCR. In parallel the expression of four housekeeping genes—SDHA (GenBank Accession No. NM_{—}004168 (SEQ. ID NO: 33); amplicon—SDHA-amplicon (SEQ. ID NO:36)), Ubiquitin (GenBank Accession No. BC000449 (SEQ. ID NO: 29); amplicon—Ubiquitin-amplicon (SEQ. ID NO: 32)), RPL19 (GenBank Accession No. NM_{—}000981 (SEQ. ID NO: 21); RPL19 amplicon (SEQ. ID NO: 24)) and TATA box (GenBank Accession No. NM_{—}003194 (SEQ. ID NO: 25); TATA amplicon (SEQ. ID NO: 28)) was measured similarly. For each RT sample, the expression of the above amplicon was normalized to the geometric mean of the quantities of the housekeeping genes. The normalized quantity of each RT sample was then divided by the median of the quantities of the ovary samples (sample numbers 19 and 20, Table 1_{—}6 above), to obtain a value of relative expression of each sample relative to median of the ovary samples.

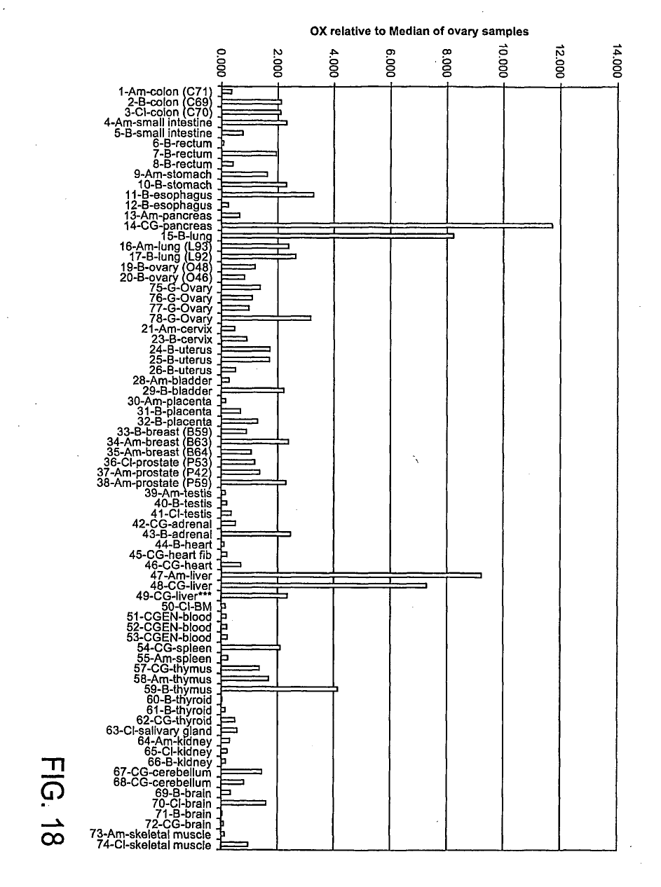

FIG. 18 is a histogram showing expression of the Homo sapiens B-factor, properdin (BF) D12115 transcripts which are detectable by amplicon as depicted in sequence name D12115_seg6 (SEQ. ID NO: 149) in different normal samples.

Expression of Homo sapiens B-factor, properdin (BF) D12115 transcripts which are detectable by amplicon as depicted in sequence name D12115_seg6 (SEQ. ID NO: 149) in normal and cancerous Ovary tissues

Expression of Homo sapiens B-factor, properdin (BF) transcripts detectable by or according to seg6-D12115_seg6 (SEQ. ID NO: 149) amplicon and primers D12115_seg6F (SEQ. ID NO: 147) and D12115_seg6R (SEQ. ID NO: 148) was measured by real time PCR. In parallel the expression of four housekeeping genes—SDHA (GenBank Accession No. NM_{—}004168 (SEQ. ID NO: 33); amplicon—SDHA-amplicon (SEQ. ID NO:36)), HPRT1 (GenBank Accession No. NM 000194 (SEQ. ID NO: 5); amplicon—HPRT1-amplicon (SEQ. ID NO: 8)), PBGD (GenBank Accession No. BC019323 (SEQ. ID NO: 1); amplicon—PBGD-amplicon (SEQ. ID NO: 4)) and GAPDH (GenBank Accession No. BC026907 (SEQ. ID NO: 9); GAPDH amplicon (SEQ. ID NO: 12)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to median of the normal PM samples.

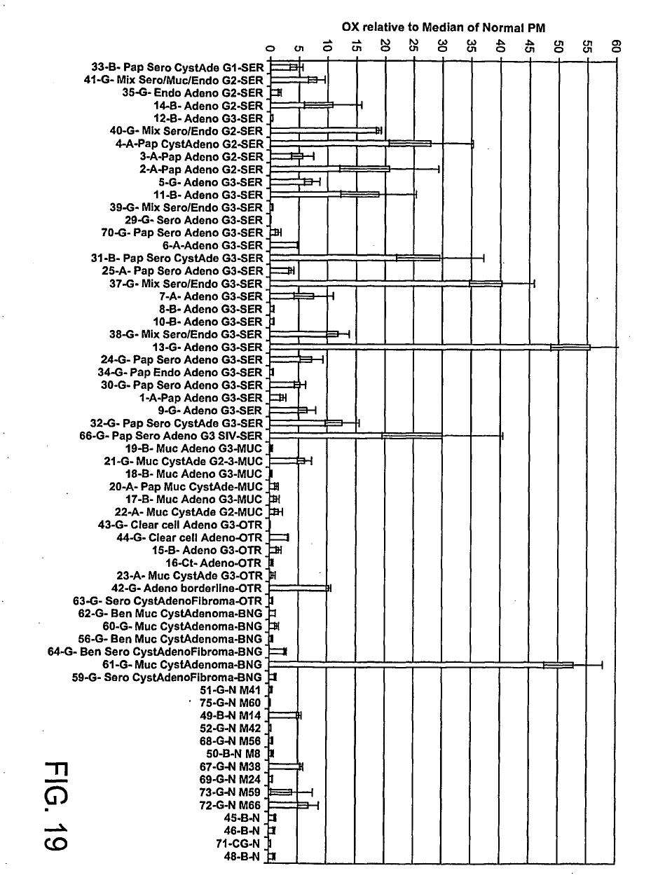

FIG. 19 is a histogram showing over expression of the above-indicated Homo sapiens B-factor, properdin (BF) transcripts in cancerous Ovary samples relative to the normal samples. Values represent the average of duplicate experiments. Error bars indicate the minimal and maximal values obtained.

As is evident from FIG. 19, the expression of Homo sapiens B-factor, properdin (BF) transcripts detectable by the above amplicon in serous carcinoma samples was significantly higher than in the non-cancerous samples (sample numbers 45, 46, 71 and 48, Table 1_{—}1 above). Notably an over-expression of at least 5 fold was found in 20 out of 43 adenocarcinoma samples, specifically in 18 out of 30 serous carcinoma samples.

Statistical analysis was applied to verify the significance of these results, as described below.

The P value for the difference in the expression levels of Homo sapiens B-factor, properdin (BF) transcripts detectable by the above amplicon in Ovary serous carcinoma samples versus the normal tissue samples was determined by T test as 1.60e-04. The P value for the difference in the expression levels of Homo sapiens B-factor, properdin (BF) transcripts detectable by the above amplicon in Ovary adenocarcinoma samples versus the normal tissue samples was determined by T test as 1.28e-04.

Threshold of 5 fold over expression was found to differentiate between serous carcinoma and normal samples with P value of 3.92e-02 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: D12115_seg6F (SEQ. ID NO: 147) forward primer; and D12115_seg6R (SEQ. ID NO: 148) reverse primer.

The present invention also preferably encompasses any amplicon obtained through the use of any suitable primer pair; for example, for the above experiment, the following amplicon was obtained as a non-limiting illustrative example only of a suitable amplicon: D12115_seg6 (SEQ. ID NO: 149).

Expression of Homo sapiens B-factor, properdin (BF) D12115 transcripts which are detectable by amplicon as depicted in sequence name D12115_seg40WT (SEQ. ID NO: 152) in different normal tissues

Expression of Homo sapiens B-factor, properdin (BF) transcripts detectable by or according to seg40WT-D12115_seg40WT (SEQ. ID NO: 152) amplicon and primers D12115_seg40WTF (SEQ. ID NO: 150) and D12115_seg40WTR (SEQ. ID NO: 151) was measured by real time PCR. In parallel the expression of four housekeeping genes—SDHA (GenBank Accession No. NM_{—}004168 (SEQ. ID NO: 33); amplicon—SDHA-amplicon (SEQ. ID NO:36)), Ubiquitin (GenBank Accession No. BC000449 (SEQ. ID NO: 29); amplicon—Ubiquitin-amplicon (SEQ. ID NO: 32)), RPL19 (GenBank Accession No. NM_{—}000981 (SEQ. ID NO: 21); RPL19 amplicon (SEQ. ID NO: 24)) and TATA box (GenBank Accession No. NM_{—}003194 (SEQ. ID NO: 25); TATA amplicon (SEQ. ID NO: 28)) was measured similarly. For each RT sample, the expression of the above amplicon was normalized to the geometric mean of the quantities of the housekeeping genes. The normalized quantity of each RT sample was then divided by the median of the quantities of the ovary samples (sample numbers 19 and 20, Table 1_{—}6 above), to obtain a value of relative expression of each sample relative to median of the ovary samples.

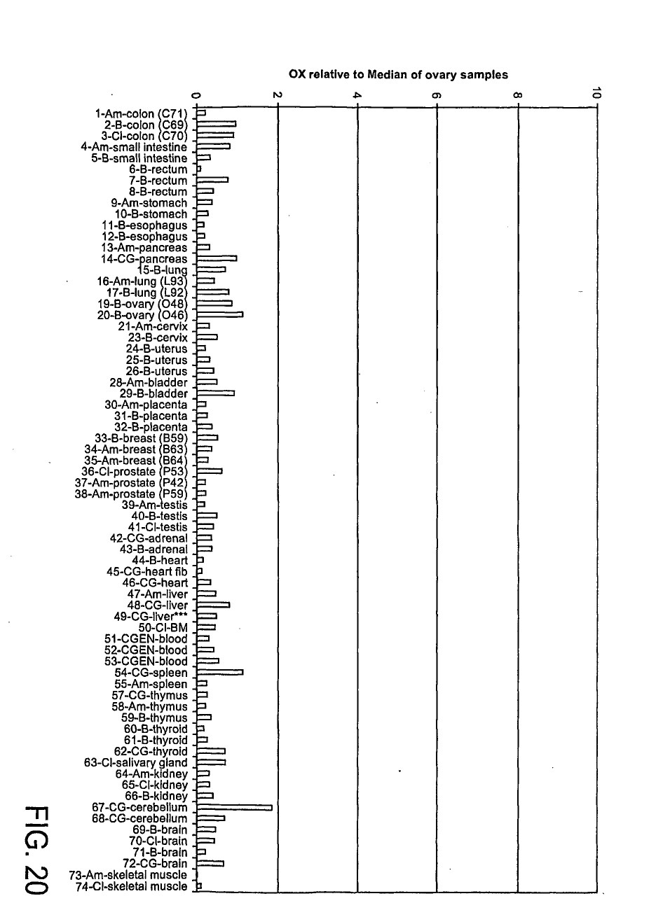

FIG. 20 is a histogram showing expression of the Homo sapiens B-factor, properdin (BF) D12115 transcripts which are detectable by amplicon as depicted in sequence name D12115 seg40WT (SEQ. ID NO: 152) in different normal samples.

Expression of Homo sapiens B-factor, properdin (BF) D12115 transcripts which are detectable by amplicon as depicted in sequence name D12115_seg46-47 (SEQ. ID NO: 155) in different normal tissues

Expression of Homo sapiens B-factor, properdin (BF) transcripts detectable by or according to seg46-47-D12115_seg46-47 (SEQ. ID NO: 155) amplicon and primers D12115_seg46-47F (SEQ. ID NO: 153) and D12115_seg46-47R (SEQ. ID NO: 154) was measured by real time PCR. In parallel the expression of four housekeeping genes—SDHA (GenBank Accession No. NM_{—}004168 (SEQ. ID NO: 33); amplicon—SDHA-amplicon (SEQ. ID NO:36), Ubiquitin (GenBank Accession No. BC000449 (SEQ. ID NO: 29); amplicon—Ubiquitin-amplicon (SEQ. ID NO: 32)), RPL19 (GenBank Accession No. NM_{—}000981 (SEQ. ID NO: 21); RPL19 amplicon (SEQ. ID NO: 24)) and TATA box (GenBank Accession No. NM 003194 (SEQ. ID NO: 25); TATA amplicon (SEQ. ID NO: 28)) was measured similarly. For each RT sample, the expression of the above amplicon was normalized to the geometric mean of the quantities of the housekeeping genes. The normalized quantity of each RT sample was then divided by the median of the quantities of the ovary samples (sample numbers 19 and 20, Table 1_{—}6 above), to obtain a value of relative expression of each sample relative to median of the ovary samples.

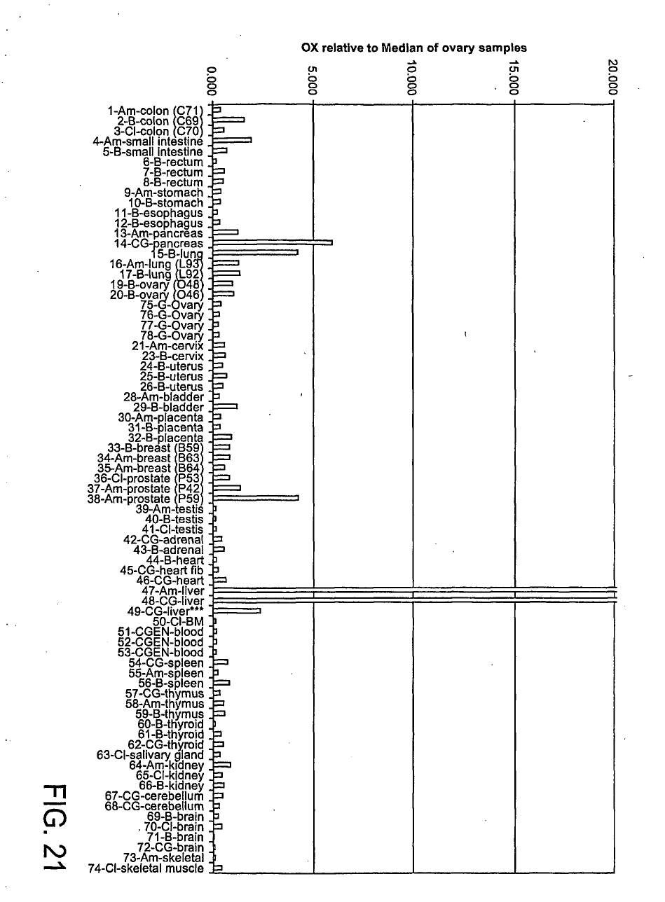

FIG. 21 is a histogram showing expression of the Homo sapiens B-factor, properdin (BF)

D12115 transcripts which are detectable by amplicon as depicted in sequence name D12115_seg46-47 (SEQ. ID NO: 155) in different normal samples.

Expression of Homo sapiens B-factor, properdin (BF) D12115 transcripts which are detectable by amplicon as depicted in sequence name D12115_seg46-47 (SEQ. ID NO: 155) in normal and cancerous Ovary tissues

Expression of Homo sapiens B-factor, properdin (BF) transcripts detectable by or according to seg46-47-D12115_seg46-47 (SEQ. ID NO: 155) amplicon and primers D12115_seg46-47F (SEQ. ID NO: 153) and D12115_seg46-47R (SEQ. ID NO: 154) was measured by real time PCR. In parallel the expression of four housekeeping genes—SDHA (GenBank Accession No. NM_{—}004168 (SEQ. ID NO: 33); amplicon—SDHA-amplicon (SEQ. ID NO:36)), HPRT1 (GenBank Accession No. NM_{—}000194 (SEQ. ID NO: 5); amplicon—HPRTI-amplicon (SEQ. ID NO: 8)), PBGD (GenBank Accession No. BC019323 (SEQ. ID NO: 1); amplicon—PBGD-amplicon (SEQ. ID NO: 4)) and GAPDH (GenBank Accession No. BC026907 (SEQ. ID NO: 9); GAPDH amplicon (SEQ. ID NO: 12)) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to median of the normal PM samples.

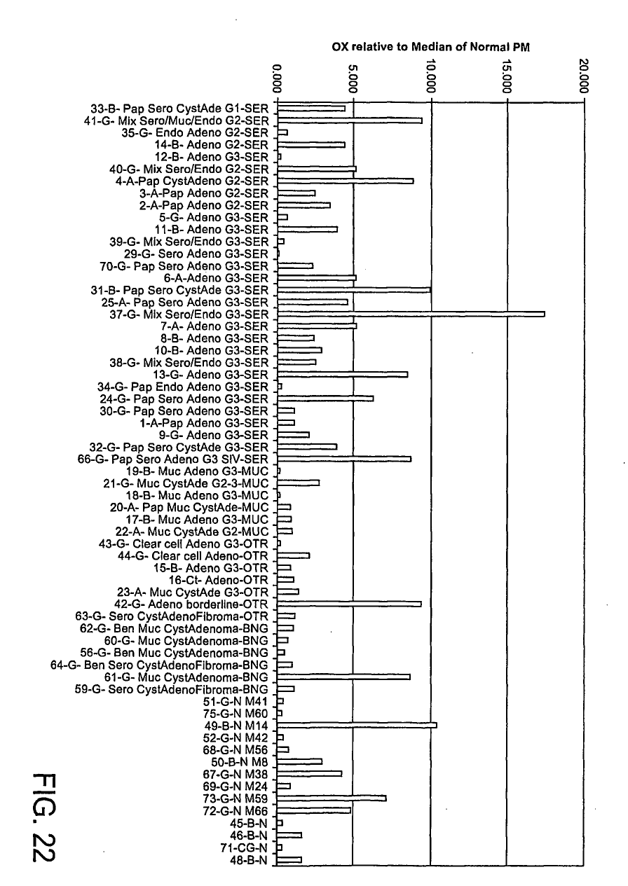

FIG. 22 is a histogram showing over expression of the above-indicated Homo sapiens B-factor, properdin (BF) transcripts in cancerous Ovary samples relative to the normal samples.

As is evident from FIG. 22, the expression of Homo sapiens B-factor, properdin (BF) transcripts detectable by the above amplicon in serous carcinoma samples was higher than in the non-cancerous samples (sample numbers 45, 46, 71 and 48, Table 1_{—}1 above). Notably an over-expression of at least 5 fold was found in 11 out of 43 adenocarcinoma samples, specifically in 10 out of 30 serous carcinoma samples.

Statistical analysis was applied to verify the significance of these results, as described below.

The P value for the difference in the expression levels of Homo sapiens B-factor, properdin (BF) transcripts detectable by the above amplicon in Ovary serous carcinoma samples versus the normal tissue samples was determined by T test as 3.19e-04. The P value for the difference in the expression levels of Homo sapiens B-factor, properdin (BF) transcripts detectable by the above amplicon in Ovary adenocarcinoma samples versus the normal tissue samples was determined by T test as 1.11e-03.

The above values demonstrate statistical significance of the results.

Primer pairs are also optionally and preferably encompassed within the present invention; for example, for the above experiment, the following primer pair was used as a non-limiting illustrative example only of a suitable primer pair: D12115_seg46-47F (SEQ. ID NO: 153) forward primer; and D12115_seg46-47R (SEQ. ID NO: 154) reverse primer.

The present invention also preferably encompasses any amplicon obtained through the use of any suitable primer pair; for example, for the above experiment, the following amplicon was obtained as a non-limiting illustrative example only of a suitable amplicon: D12115_seg46-47 (SEQ. ID NO: 155).

Expression of Homo sapiens B-factor, properdin (BF) D12115 transcripts which are detectable by amplicons as depicted in sequence names D12115seg4 (SEQ. ID NO: 146) and D12115seg6 (SEQ. ID NO: 149) in normal and cancerous colon tissues

Expression of Homo sapiens B-factor, properdin (BF) transcripts detectable by or according to seg4 and seg6-D12115seg4 (SEQ. ID NO: 146) and D12115seg6 (SEQ. ID NO: 149) amplicons and primers D12115seg4F (SEQ. ID NO: 144), D12115seg4R (SEQ. ID NO: 145), D12115seg6F (SEQ. ID NO: 147) and D12115seg6R (SEQ. ID NO: 148) was measured by real time PCR. In parallel expression of several housekeeping genes as detailed in “Materials and Experimental Procedures” section herein, was measured similarly. For each RT sample, the expression of the above amplicons was normalized to the normalization factor calculated from the expression of the house keeping genes that were selected for colon tissue panel, as described in normalization method 2 in the “materials and methods” section. The normalized quantity of each RT sample was then divided by the median of the quantities of the normal samples of colon panel (Table 1_{—}4 above), to obtain a value of fold differential expression for each sample relative to median of the normal samples.

In one experiment that was carried out with D12115seg4 (SEQ. ID NO: 146) and D12115seg6 (SEQ. ID NO: 149) no differential expression in the colon cancerous samples relative to the normal samples was observed.

Expression of Homo sapiens B-factor, properdin (BF) D12115 transcripts which are detectable by amplicons as depicted in sequence names D12115seg27 (SEQ. ID NO: 320) and D12115seg34 (SEQ. ID NO: 323) in normal and cancerous tissues

Expression of Homo sapiens B-factor, properdin (BF) transcripts detectable by or according to seg27 and seg34-D12115seg27 (SEQ. ID NO: 320) and D12115seg34 (SEQ. ID NO: 323) amplicons and primers D12115seg27F (SEQ. ID NO: 318), D12115seg27R (SEQ. ID NO: 319), D12115seg34F (SEQ. ID NO: 321) and D12115seg34R (SEQ. ID NO: 322) was measured by real time PCR. In parallel expression of several housekeeping genes, as detailed in “Materials and Experimental Procedures” section above, was measured similarly. For each RT sample, the expression of the above amplicons was normalized to the normalization factor calculated from the expression of the house keeping genes that were selected for the relevant tissue panel, as described in normalization method 2 in the “materials and methods” section. The normalized quantity of each RT sample was then divided by the median of the quantities of the normal samples of the relevant panel (Tables 1_{—}4, 1_{—}3, 1_{—b 1} and 1_{—}2 above), to obtain a value of fold differential expression for each sample relative to median of the normal samples.

In one experiment that was carried out with D12115seg27 (SEQ. ID NO: 320) and D12115seg34 (SEQ. ID NO: 323) no differential expression in the colon cancerous samples relative to the normal samples was observed.

In one experiment that was carried out with D12115seg27 (SEQ. ID NO: 320) and D12115seg34 (SEQ. ID NO: 323) no differential expression in the breast cancerous samples relative to the normal samples was observed.

In one experiment that was carried out with D12115seg27 (SEQ. ID NO: 320) and D12115seg34 (SEQ. ID NO: 323) no differential expression in the ovary cancerous samples relative to the normal samples was observed.

In one experiment that was carried out with D12115seg27 (SEQ. ID NO: 320) and D12115seg34 (SEQ. ID NO: 323) no differential expression in the lung cancerous samples relative to the normal samples was observed.

### Description for Cluster C03950

Cluster C03950 features 15 transcript(s) and 38 segment(s) of interest, the names for which are given in Tables 85 and 86, respectively. The selected protein variants are given in table 87.

TABLE 85
Transcripts of interest
Transcript Name

C03950_3_T2 (SEQ. ID NO: 156)
C03950_3_T4 (SEQ. ID NO: 157)
C03950_3_T7 (SEQ. ID NO: 158)
C03950_3_T8 (SEQ. ID NO: 159)
C03950_3_T9 (SEQ. ID NO: 160)
C03950_3_T10 (SEQ. ID NO: 161)
C03950_3_T11 (SEQ. ID NO: 162)
C03950_3_T13 (SEQ. ID NO: 163)
C03950_3_T15 (SEQ. ID NO: 164)
C03950_3_T17 (SEQ. ID NO: 165)
C03950_3_T18 (SEQ. ID NO: 166)
C03950_3_T19 (SEQ. ID NO: 167)
C03950_3_T21 (SEQ. ID NO: 168)
C03950_3_T22 (SEQ. ID NO: 169)
C03950_3_T23 (SEQ. ID NO: 170)

TABLE 86
Segments of interest
Segment Name

C03950_3_N2 (SEQ. ID NO: 171)
C03950_3_N6 (SEQ. ID NO: 172)
C03950_3_N23 (SEQ. ID NO: 173)
C03950_3_N27 (SEQ. ID NO: 174)
C03950_3_N33 (SEQ. ID NO: 175)
C03950_3_N44 (SEQ. ID NO: 176)
C03950_3_N45 (SEQ. ID NO: 177)
C03950_3_N48 (SEQ. ID NO: 178)
C03950_3_N49 (SEQ. ID NO: 179)
C03950_3_N56 (SEQ. ID NO: 180)
C03950_3_N62 (SEQ. ID NO: 181)
C03950_3_N63 (SEQ. ID NO: 182)
C03950_3_N67 (SEQ. ID NO: 183)
C03950_3_N71 (SEQ. ID NO: 184)
C03950_3_N77 (SEQ. ID NO: 185)
C03950_3_N0 (SEQ. ID NO: 186)
C03950_3_N4 (SEQ. ID NO: 187)
C03950_3_N9 (SEQ. ID NO: 188)
C03950_3_N13 (SEQ. ID NO: 189)
C03950_3_N15 (SEQ. ID NO: 190)
C03950_3_N17 (SEQ. ID NO: 191)
C03950_3_N19 (SEQ. ID NO: 192)
C03950_3_N21 (SEQ. ID NO: 193)
C03950_3_N29 (SEQ. ID NO: 194)
C03950_3_N31 (SEQ. ID NO: 195)
C03950_3_N35 (SEQ. ID NO: 196)
C03950_3_N37 (SEQ. ID NO: 197)
C03950_3_N39 (SEQ. ID NO: 198)
C03950_3_N40 (SEQ. ID NO: 199)
C03950_3_N42 (SEQ. ID NO: 200)
C03950_3_N47 (SEQ. ID NO: 201)
C03950_3_N51 (SEQ. ID NO: 202)
C03950_3_N58 (SEQ. ID NO: 203)
C03950_3_N60 (SEQ. ID NO: 204)
C03950_3_N65 (SEQ. ID NO: 205)
C03950_3_N69 (SEQ. ID NO: 206)
C03950_3_N73 (SEQ. ID NO: 207)
C03950_3_N75 (SEQ. ID NO: 208)

TABLE 87
Proteins of interest

| Protein Name | Corresponding Transcript(s) |
|---|---|
| C03950_3_P5 | C03950_3_T2 (SEQ. ID NO: 156) |
| (SEQ. ID NO: 212) | |
| C03950_3_P7 | C03950_3_T4 (SEQ. ID NO: 157) |
| (SEQ. ID NO: 213) | |
| C03950_3_P9 | C03950_3_T7 (SEQ. ID NO: 158) |
| (SEQ. ID NO: 214) | |
| C03950_3_P10 | C03950_3_T8 (SEQ. ID NO: 159) |
| (SEQ. ID NO: 215) | |
| C03950_3_P11 | C03950_3_T9 (SEQ. ID NO: 160) |
| (SEQ. ID NO: 216) | |
| C03950_3_P12 | C03950_3_T10 (SEQ. ID NO: 161) |
| (SEQ. ID NO: 217) | |
| C03950_3_P13 | C03950_3_T11 (SEQ. ID NO: 162) |
| (SEQ. ID NO: 218) | |
| C03950_3_P15 | C03950_3_T13 (SEQ. ID NO: 163) |
| (SEQ. ID NO: 219) | |
| C03950_3_P17 | C03950_3_T15 (SEQ. ID NO: 164) |
| (SEQ. ID NO: 220) | |
| C03950_3_P19 | C03950_3_T17 (SEQ. ID NO: 165) |
| (SEQ. ID NO: 221) | |
| C03950_3_P20 | C03950_3_T18 (SEQ. ID NO: 166) |
| (SEQ. ID NO: 222) | |
| C03950_3_P21 | C03950_3_T19 (SEQ. ID NO: 167) |
| (SEQ. ID NO: 223) | |
| C03950_3_P23 | C03950_3_T21 (SEQ. ID NO: 168) |
| (SEQ. ID NO: 224) | |
| C03950_3_P24 | C03950_3_T22 (SEQ. ID NO: 169) |
| (SEQ. ID NO: 225) | |
| C03950_3_P25 | C03950_3_T23 (SEQ. ID NO: 170) |
| (SEQ. ID NO: 226) | |
| C03950_3_P28 | C03950_3_T2 (SEQ. ID NO: 156) |
| (SEQ. ID NO: 227) | |
| C03950_3_P31 | C03950_3_T10 (SEQ. ID NO: 161) |
| (SEQ. ID NO: 228) | |
| C03950_3_P33 | C03950_3_T13 (SEQ. ID NO: 163) |
| (SEQ. ID NO: 229) | |
| C03950_3_P35 | C03950_3_T18 (SEQ. ID NO: 166) |
| (SEQ. ID NO: 230) | |

These sequences are variants of the known protein Serine/threonine-protein kinase TNNI3K (SEQ. ID NO:209) (SwissProt accession identifier TNI3K_HUMAN (SEQ. ID NO: 396); known also according to the synonyms EC 2.7.1.37; TNNI3—interacting kinase; Cardiac ankyrin repeat kinase), referred to herein as the previously known protein.

The variants CO3950_T10 (SEQ. ID NO: 161) and C03950_T18 (SEQ. ID NO: 166) were previously disclosed by the inventors in published PCT application no WO0603527, and the variants C03950_T17 (SEQ. ID NO: 165), C03950_P19 (SEQ. ID NO: 221) and C03950_P35 (SEQ. ID NO: 230) were previously disclosed by the inventors in published PCT application no WO2005/071058, hereby incorporated by reference as if fully set forth herein, but have now been shown to have novel and surprising diagnostic uses as described herein for other variants of cluster C03950.

According to optional but preferred embodiments of the present invention, variants of this cluster according to the present invention (amino acid and/or nucleic acid sequences of C03950) may optionally have one or more of the following utilities. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

A non-limiting example of such a utility is the detection, diagnosis and/or determination of risk of melanoma. The method comprises detecting a C03950 variant, for example a variant protein, protein fragment, peptide, polynucleotide, polynucleotide fragment and/or oligonucleotide as described herein, optionally and preferably in a serum sample. The expression levels of the C03950 variant as determined in a patient can be further compared to those in a normal individual.

Polymorphic variants of the known Serine/threonine-protein kinase TNNI3K (SEQ. ID NO:209) is described with regard to PCT Application No. WO 05/017176, hereby incorporated by reference as if fully set forth herein. These variants were shown to be related to risk of melanoma.

Another non-limiting example of such a utility is the detection, diagnosis and/or determination of lymphoma, optionally including prediction of survival. The method comprises detecting a C03950 variant, for example a variant protein, protein fragment, peptide, polynucleotide, polynucleotide fragment and/or oligonucleotide as described herein, optionally and preferably in a serum sample. The expression levels of the C03950 variant as determined in a patient can be further compared to those in a normal individual.

Expression level of the known Serine/threonine-protein kinase TNNI3K (SEQ. ID NO:209) is described with regard to PCT Application No. WO 05/024043, hereby incorporated by reference as if fully set forth herein. The expression level, measured by using microarrays, was shown to be related to risk of lymphoma, including but not limited to follicular lymphoma, diffuse large B cell lymphoma or mantle cell lymphoma.

Another non-limiting example of such a utility is the detection, diagnosis and/or determination of lung cancer, optionally including prediction of survival, including but not limited to small cell lung carcinoma (oat cell carcinoma), or non-small cell carcinomas (e.g., squamous cell carcinoma, adenocarcinoma, large cell lung carcinoma, carcinoid, granulomatous). The method comprises detecting a C03950 variant, for example a variant protein, protein fragment, peptide, polynucleotide, polynucleotide fragment and/or oligonucleotide as described herein, optionally and preferably in a serum sample. The expression levels of the C03950 variant as determined in a patient can be further compared to those in a normal individual.

Expression level of the known Serine/threonine-protein kinase TNNI3K (SEQ. ID NO:209) is described with regard to PCT Application No. WO 02/086443, hereby incorporated by reference as if fully set forth herein. The expression level, measured by using the Eos/Afiymetrix Hu03 Genechip array, was shown to be related to lung cancer, including but not limited to early detection of lung cancers, monitoring and early detection of relapse following treatment of lung cancers, monitoring response to therapy of lung cancers, determining prognosis of lung cancers, directing therapy of lung cancers, selecting patients for postoperative chemotherapy or radiation therapy, selecting therapy, determining tumor prognosis, treatment, or response to treatment, and early detection of precancerous lesions of the lung. Examples of benign or precancerous lesions include but are not limited to atelectasis, emphysema, brochitis, chronic obstructive pulmonary disease, fibrosis, I hypersensitivity pneumonitis (HP), interstitial pulmonary fibrosis (IPF), asthma, and bronchiectasis.

Protein Serine/threonine-protein kinase TNNI3K (SEQ. ID NO:209) is known or believed to have a role in cardiac physiology. The sequence for protein Serine/threonine-protein kinase TNNI3K (SEQ. ID NO:209) is given. Known polymorphisms for this sequence are as shown in Table 88.

TABLE 88
Amino acid mutations for Known Protein

| SNP position(s) on | |
|---|---|
| amino acid sequence | Comment |
| 228 | I -> V |
| 351 | I -> M |
| 468 | N -> S |
| 730 | R -> L |

Protein Serine/threonine-protein kinase TNNI3K (SEQ. ID NO:209) localization is believed to be Nuclear Expressed at lower levels in the cytoplasm.

Other non-limiting exemplary utilities for CO3950 variants according to the present invention are described in greater detail below and also with regard to the previous section on clinical utility.

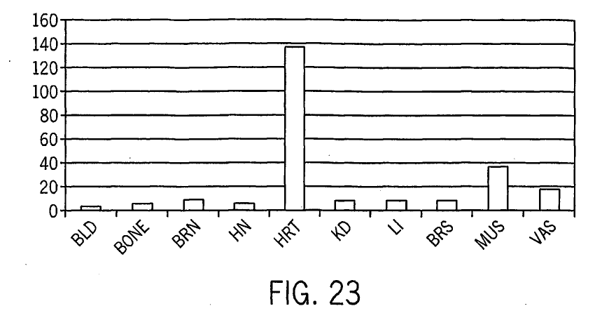

The heart-selective diagnostic marker prediction engine provided the following results with regard to cluster C03950. Predictions were made for selective expression of transcripts of this contig in heart tissue, according to the previously described methods. The'numbers on the y-axis of FIG. 23 below refer to weighted expression of ESTs in each category, as “parts per million” (ratio of the expression of ESTs for a particular cluster to the expression of all ESTs in that category, according to parts per million).

Overall, the following results were obtained as shown with regard to the histogram in FIG. 23, concerning the number of heart-specific clones in libraries/sequences.

This cluster was found to be selectively expressed in heart for the following reasons: in a comparison of the ratio of expression of the cluster in heart specific ESTs to the overall expression of the cluster in non-heart ESTs, which was found to be 9.5; the ratio of expression of the cluster in heart specific ESTs to the overall expression of the cluster in muscle-specific ESTs which was found to be 3.7; and fisher exact test P-values were computed both for library and weighted clone counts to check that the counts are statistically significant, and were found to be 1.40E-03.

One particularly important measure of specificity of expression of a cluster in heart tissue is the previously described comparison of the ratio of expression of the cluster in heart as opposed to muscle. This cluster was found to be specifically expressed in heart as opposed to non-heart ESTs as described above. However, many proteins have been shown to be generally expressed at a higher level in both heart and muscle, which is less desirable. For this cluster, as described above, the ratio of expression of the cluster in heart specific ESTs to the overall expression of the cluster in muscle-specific ESTs which was found to be 9.5, which clearly supports specific expression in heart tissue.

As noted above, cluster C03950 features 15 transcript(s), which were listed in Table 85 above. These transcript(s) encode for protein(s) which are variant(s) of protein Serine/threonine-protein kinase TNNI3K (SEQ. ID NO:209). A description of each variant protein according to the present invention is now provided.

Variant protein C03950_{—}3_P5 (SEQ. ID NO:212) according to the present invention is encoded by transcript C03950_{—}3_T2 (SEQ. ID NO:156). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

### 1. Comparison Report Between C03950_{—}3_P5 (SEQ. ID NO:212) and TNI3K_HUMAN (SEQ. ID NO: 396):

A. An isolated chimeric polypeptide encoding for C03950_{—}3_P5 (SEQ. ID NO:212), comprising a first amino acid sequence being at least 70%, optionally at least 80%, preferably at least 85%, more preferably at least 90% and most preferably at least 95%, homologous to a polypeptide having the sequence AVRRGLREGGA (SEQ. ID NO: 342) corresponding to amino acids 1-11 of C03950_{—}3_P5 (SEQ. ID NO:212), a second amino acid sequence being at least 90% homologous to MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTTLLIHSDEWKKKVSES YVITIERLEDDLQIKEKELTELRNIFGSDEAFSKVNLNYRTENGLSLLHLCCICGGKKSHIRTLM LKGLRPSRLTRNGFTALHLAVYKDNAELITSLLHSGADIQQVGYGGLTALHEATTAGHLEAAD VLLQHGANVNIQDAVFFTPLHIAAYYGHEQVTRLLLKFGADVNVSGEVGDRPLHLASAKGFL NIAKLLMEEGSKADVNAQDNEDHVPLHFCSRFGHHDIVKYLLQSDLEVQPHVVNIYGDTPLH LACYNGKFEVAKELPISGTESLTKENIFSETAFHSACTYGKSIDLVKFLLDQNVININHQGRDG HTGLHSACYFIGHIRLVQFLLDNGADMNLVACDPSRSSGEKDEQTCLMWAYEKGBDAIVTLL KHYKRPQDELPCNEYSQPGGDGSYVSVPSPLGKIKSMTKEKADILLLRAGLPSHFHLQLSEIEF HEIIGSGSFGKVYKGRCRNKIVAIKRYRANTYCSKSDVDMFCREVSILCQLNIAPCVIQFVGAC LNDPSQFANTQYISGGSLFSLLHEQKRILDLQSKLIIAVDVAKGMEYLHNLTQPIIHRDLN corresponding to amino acids 1-691 of TNI3K HUMAN (SEQ. ID NO: 396), which also corresponds to amino acids 12-702 of C03950_{—}3_P5 (SEQ. ID NO:212), a third amino acid sequence being at least 70%, optionally at least 80%, preferably at least 85%, more preferably at least 90% and most preferably at least 95%, homologous to a polypeptide having the sequence RSAITSRIWITHSICIWRGAHYFNREECNFRCMLTSAILK corresponding to amino acids 703-742 of C03950_{—}3_P5 (SEQ. ID NO:212), and a fourth amino acid sequence being at least 90% homologous to ESRFLQSLDEDNMTKQPGNLRWMAPEVFTQCTRYTIKADVESYALCLWEILTGELPFAHLKPA AAAADMAYHHIRPPIGYSIPKPISSLLIRGWNACPEGRPEFSEVVMKLEECLCNIELMSPASSNS SGSLSPSSSSDCLVNRGGPGRSHVAALRSRFELEYALNARSYAALSQSAGQYSSQGLSLEEMK RSLQYTPIDKYGYVSDPMSSMHFHSCRNSSSFEDSS corresponding to amino acids 710-936 of TNI3K_HUMAN (SEQ. ID NO: 396), which also corresponds to amino acids 743-969 of C03950_{—}3_P5 (SEQ. ID NO:212), wherein said first amino acid sequence, second amino acid sequence, third amino acid sequence and fourth amino acid sequence are contiguous and in a sequential order.

B. An isolated polypeptide encoding for a head of C03950_{—}3_P5 (SEQ. ID NO:212), comprising a polypeptide being at least 70%, optionally at least about 80%, preferably at least about 85%, more preferably at least about 90% and most preferably at least about 95% homologous to the sequence AVRRGLREGGA (SEQ. ID NO: 342) of C03950_{—}3_P5 (SEQ. ID NO:212).

### 2. Comparison Report Between C03950_{—}3_P5 (SEQ. ID NO:212) and NP_{—}057062 (SEQ. ID NO:210):

A. An isolated chimeric polypeptide encoding for C03950_{—}3_P5 (SEQ. ID NO:212), comprising a first amino acid sequence being at least 70%, optionally at least 80%, preferably at least 85%, more preferably at least 90% and most preferably at least 95%, homologous to a polypeptide having the sequence AVRRGLREGGAMAAARDPPEVSLREATQRKLRRFSELRGKLVARGEFWDIVAITAADEKQEL AYNQQLSEKLKRKELPLGVQYHVPVDPAGAKIGNGGSTLCALQCLEKLYGDKWNSFTILLIH S (SEQ. ID NO: 343) corresponding to amino acids 1-125 of C03950_{—}3_P5 (SEQ. ID NO:212), a second amino acid sequence being at least 90% homologous to DEWKKKVSESYVITIERLEDDLQIKEKELTELRNIFGSDEAFSKVNLNYRTENGLSLLHLCCIC GGKKSHIRTLMLKGLRPSRLTRNGFTALHLAVYKDNAELITSLLHSGADIQQVGYGGLTALHI ATIAGHLEAADVLLQHGANVNIQDAVFFTPLHLAAYYGHEQVTRLLLKFGADVNVSGEVGDR PLHLASAKGFLNIAKLLMEEGSKADVNAQDNEDHVPLHFCSRFGHHDIVKYLLQSDLEVQPH VVNIYGDTPLHLACYNGKPEVAKEIIQISGTESLTKENIFSETAFHSACTYGKSIDLVKFLLDQN VININHQGRDGHTGLHSACYHGHIRLVQFLLDNGADMNLVACDPSRSSGEKDEQTCLMWAY EKGHDAIVTLLKHYKRPQDELPCNEYSQPGGDGSYVSVPSPLGKIKSMTKEKADILLLRAGLP SHFHLQLSEIEFIIEIIGSGSFGKVYKGRCRNKIVAIKRYRANTYCSKSDVDMFCREVSILCQLN HPCVIQFVGACLNDPSQFATVTQYISGGSLFSLLHEQKRILDLQSKLBAVDVAKGMEYLHNLT QPIIHRDLN corresponding to amino acids 14-590 of NP_{—}057062 (SEQ. ID NO:210), which also corresponds to amino acids 126-702 of C03950_{—}3_P5 (SEQ. ID NO:212), a third amino acid sequence being at least 70%, optionally at least 80%, preferably at least 85%, more preferably at least 90% and most preferably at least 95%, homologous to a polypeptide having the sequence RSAITSRIWITHSICIWRGAHYPNREECNFRCNILTSAELK corresponding to amino acids 703-742 of C03950_{—}3_P5 (SEQ. ID NO:212), and a fourth amino acid sequence being at least 90% homologous to ESRFLQSLDEDNMTKQPGNLRWMAPEVFTQCTRYTIKADVFSYALCLWEILTGETPFABLKPA AAAADMAYHHIRPPIGYSJPKPISSLLIRGWNACPEGRPEFSEVVMKLEECLCNIELMSPASSNS SGSLSPSSSSDCLVNRGGPGRSHVAALRSRFELEYALNARSYAALSQSAGQYSSQGLSLEEMK RSLQYTPIDKYGYVSDPMSSMHFHSCRNSSSFEDSS corresponding to amino acids 609-835 of NP_{—}057062 (SEQ. ID NO:210), which also corresponds to amino acids 743-969 of C03950_{—}3_P5 (SEQ. ID NO:212), wherein said first amino acid sequence, second amino acid sequence, third amino acid sequence and fourth amino acid sequence are contiguous and in a sequential order.

B. An isolated polypeptide encoding for a head of C03950_{—}3_P5 (SEQ. ID NO:212), comprising a polypeptide being at least 70%, optionally at least about 80%, preferably at least about 85%, more preferably at least about 90% and most preferably at least about 95% homologous to the sequence AVRRGLREGGAMAAARDPPEVSLREATQRKLRRFSELRGKLVARGEFWDIVAITAADEKQEL AYNQQLSEKLKRKELPLGVQYHVFVDPAGAKIGNGGSTLCALQCLEKLYGDKWNSFTILLIH S (SEQ. ID NO: 343) of C03950_{—}3_P5 (SEQ. ID NO:212).

### 3. Comparison Report Between C03950_{—}3_P5 (SEQ. ID NO:212) and Q9Y2V6_HUMAN (SEQ. ID NO:210):

A. An isolated chimeric polypeptide encoding for C03950_{—}3_P5 (SEQ. ID NO:212), comprising a first amino acid sequence being at least 70%, optionally at least 80%, preferably at least 85%, more preferably at least 90% and most preferably at least 95%, homologous to a polypeptide the sequence AVRRGLREGGAMAAARDPPEVSLREATQRKLRRFSELRGKLVARGEFWDIVAITAADEKQEL AYNQQLSEKLKRKELPLGVQYHVFVDPAGAKIGNGGSTLCALQCLEKLYGDKWNSFTILLIH S (SEQ. ID NO: 343) corresponding to amino acids 1-125 of C03950_{—}3_P5 (SEQ. ID NO:212), second amino acid sequence being at least 90% homologous to DEWKKKVSESYVITIERLEDDLQIKEKELTELRNLFGSDEAFSKVNLNYRTENGLSLLHLCCIC GGKKSHIRTLMLKGLRPSRLTRNGFTALHLAVYKDNAELITSLLHSGADIQQVGYGGLTALHE ATIAGHLEAADVLLQHGANVNIQDAVFFTPLHIAAYYGHEQVTRLLLKFGADVNVSGEVGDR PLHLASAKGFLNIAKLLMEEGSKADVNAQDNEDHVPLHFCSRFGHHDIVKYLLQSDLEVQPH VVNIYGDTPLHLACYNGKFEVAKEIIQISGTESLTKENIFSETAFHSACTYGKSLDLVKFLLDQN VININHQGRDGHTGLHSACYHGHIRLVQFLLDNGADMNLVACDPSRSSGEKDEQTCLMWAY EKGHDAIVTLLKHYKRPQDELPCNEYSQPGGDGSYVSVPSPLGKIKSMTKEKADILLLRAGLP SHFHLQLSEIEFHEIIGSGSFGKVYKGRCRNKIVAIKRYRANTYCSKSDVDMFCREVSILCQLN HPCVIQFVGACLNDPSQFAIVTQYISGGSLFSLLHEQKRILDLQSKLIIAVDVAKGMEYLHNLT QPIJHRDLN corresponding to amino acids 14-590 of Q9Y2V6_HUMAN (SEQ. ID NO:210), which also corresponds to amino acids 126-702 of C03950_{—}3_P5 (SEQ. ID NO:212), a third amino acid sequence being at least 70%, optionally at least 80%, preferably at least 85%, more preferably at least 90% and most preferably at least 95%, homologous to a polypeptide having the sequence RSAITSRIWITHSICIWRGAHYFNREECNFRCMLTSAILK corresponding to amino acids 703-742 of C03950_{—}3_P5 (SEQ. ID NO:212), and a fourth amino acid sequence being at least 90% homologous to ESRFLQSLDEDNMTKQPGNLRWMAPEVFTQCTRYTEKADVFSYALCLWEILTGEIPFAHLKPA AAAADMAYIIHIRPPIGYSIPKPISSLLIRGWNACPEGRPEFSEVVMKLEECLCNIELMSPASSNS SGSLSPSSSSDCLVNRGGPGRSHVAALRSRFELEYALNARSYAALSQSAGQYSSQGLSLEEMK RSLQYTPIDKYGYVSDPMSSMITFHSCRNSSSFEDSS corresponding to amino acids 609-835 of Q9Y2V6_HUMAN (SEQ. ID NO:210), which also corresponds to amino acids 743-969 of C03950_{—}3_P5 (SEQ. ID NO:212), wherein said first amino acid sequence, second amino acid sequence, third amino acid sequence and fourth amino acid sequence are contiguous and in a sequential order.

B. An isolated polypeptide encoding for a head of C03950_{—}3_P5 (SEQ. ID NO:212), comprising a polypeptide being at least 70%, optionally at least about 80%, preferably at least about 85%, more preferably at least about 90% and most preferably at least about 95% homologous to the sequence AVRRGLREGGAMAAARDPPEVSLREATQRKLRRFSELRGKLVARGEFWDIVAITAADEKQEL AYNQQLSEKLKRKELPLGVQYHVFVDPAGAKIGNGGSTLCALQCLEKLYGDKWNSFTILLM S (SEQ. ID NO: 343) of C03950_{—}3_P5 (SEQ. ID NO:212).

### 4. Comparison Report Between C03950_{—}3_P5 (SEQ. ID NO:212) and Q6MZS9_HUMAN (SEQ. ID NO:211):

A. An isolated chimeric polypeptide encoding for C03950_{—}3P5 (SEQ. ID NO:212), comprising a first amino acid sequence being at least 70%, optionally at least 80%, preferably at least 85%, more preferably at least 90% and most preferably at least 95%, homologous tom polypeptide having the sequence AVRRGLR (SEQ. ID NO: 344) corresponding to amino acids 1-7 of

C03950_{—}3_P5 (SEQ. ID NO:212), a second amino acid sequence being at least 90% homologous to EGGAMAAARDPPEVSLREATQRKLRRFSELRGKLVARGEFWDIVAITAADEKQELAYNQQLS EKLKRKELPLGVQYHVFVDPAGAKIGNGGSTLCALQCLEKLYGDKVVNSFTILLIHSDEWKKK VSESYVITIERLEDDLQIKEKELTELRNIFGSDEAFSKVNLNYRTENGLSLLHLCCICGGKKSHI RTLMLKGLRPSRLTRNGFTALHLAVYKDNAELITSLLHSGADIQQVGYGGLTALITIATIAGHL EAADVLLQHGANVNIQDAVFFTPLHIAAYYGHEQVTRLLLKFGADVNVSGEVGDRPLHLAS AKGFLNIAKLLMEEGSKADVNAQDNEDHVPLHFCSRFGHHD corresponding to amino acids 14-367 of Q6MZS9_HUMAN (SEQ. ID NO:211), which also corresponds to amino acids 8-361 of C03950_{—}3_P5 (SEQ. ID NO:212), a bridging amino acid I corresponding to amino acid 362 of C03950_{—}3_P5 (SEQ. ID NO:212), a third amino acid sequence being at least 90% homologous to VKYLLQSDLEVQPHVVNTYGDTPLHLACYNGKFEVAKEIIQISGTESLTKENIFSETAFHSACT YGKSIDLVKFLLDQNVININFIQGRDGHTGLHSACYHGHIRLVQFLLDNGADM corresponding to amino acids 369-484 of Q6MZS9_HUMAN (SEQ. ID NO:211), which also corresponds to amino acids 363-478 of C03950_{—}3_P5 (SEQ. ID NO:212), a bridging amino acid N corresponding to amino acid 479 of C03950_{—}3_P5 (SEQ. ID NO:212), a fourth amino acid sequence being at least 90% homologous to LVACDPSRSSGEKDEQTCLMWAYEKGHDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNHPCVIQFVGACLNDPSQFAIVTQYISGGSLFSLLHEQKRI LDLQSKLIIAVDVAKGMEYLHNLTQPIIHRDLNR corresponding to amino acids 486-709 of Q6MZS9_HUMAN (SEQ. ID NO:211), which also corresponds to amino acids 480-703 of C03950_{—}3P5 (SEQ. ID NO:212), and a fifth amino acid sequence being at least 70%, optionally at least 80%, preferably at least 85%, more preferably at least 90% and most preferably at least 95% homologous to a polypeptide having the sequence SAITSRIWITHSICIWRGAHYFNREECNFRCMLTSAILKESRFLQSLDEDNMTKQPGNLRWMA PEVFTQCTRYTIKADVFSYALCLWEILTGEIPFAHLKPAAAAADMAYHHIRPPIGYSIPKPISSL LIRGWNACPEGRPEFSEVVMKLEECLCNIELMSPASSNSSGSLSPSSSSDCLVNRGGPGRSHVA ALRSRFELEYALNARSYAALSQSAGQYSSQGLSLEEMKRSLQYTPIDKYGYVSDPMSSMHFH SCRNSSSFEDSS (SEQ. ID NO: 345)corresponding to amino acids 704-969 of C03950_{—}3_P5 (SEQ. ID NO:212), wherein said first amino acid sequence, second amino acid sequence, bridging amino acid, third amino acid sequence, bridging amino acid, fourth amino acid sequence and fifth amino acid sequence are contiguous and in a sequential order.

B. An isolated polypeptide encoding for a head of C03950_{—}3_P5 (SEQ. ID NO:212), comprising a polypeptide being at least 70%, optionally at least about 80%, preferably at least about 85%, more preferably at least about 90% and most preferably at least about 95% homologous to the sequence AVRRGLR (SEQ. ID NO: 344) of C03950_{—}3_P5 (SEQ. ID NO:212).

C. An isolated polypeptide encoding for an edge portion of C03950_{—}3_P5 (SEQ. ID NO:212), comprising an amino acid sequence being at least 70%, optionally at least about 80%, preferably at least about 85%, more preferably at least about 90% and most preferably at least about 95% homologous to the sequence SAITSRIWITHSICIWRGAHYFNREECNFRCMLTSAILKESRFLQSLDEDNMTKQPGNLRWMA PEVFTQCTRYTIKADVFSYALCLWEILTGEIPFAHLKPAAAAADMAYHHIRPPIGYSIPKPISSL LIRGWNACPEGRPEFSEVVMKLEECLCNIELMSPASSNSSGSLSPSSSSDCLVNRGGPGRSHVA ALRSRFELEYALNARSYAALSQSAGQYSSQGLSLEEMKRSLQYTPIDKYGYVSDPMSSMHFH SCRNSSSFEDSS (SEQ. ID NO: 345) of C03950_{—}3_P5 (SEQ. ID NO:212).

The localization of the variant protein was determined according to results from a number of different software programs and analyses, including analyses from SignalP and other specialized programs. The variant protein is believed to be located intracellularly.

Variant protein C03950_{—}3_P5 (SEQ. ID NO:212) also has the following non-silent SNPs (Single Nucleotide Polymorphisms) as listed in Table 89, (given according to their position(s) on the amino acid sequence, with the alternative amino acid(s) listed.

TABLE 89
Amino acid mutations

| SNP position(s) on amino acid | |
|---|---|
| sequence | Alternative amino acid(s) |
| 119 | T -> P |

The variant protein has the following domains, as determined by using InterPro. The domains are described in Table 90:

TABLE 90
InterPro domain(s)

| Domain | | |
|---|---|---|
| description | Analysis type | Position(s) on protein |
| Protein kinase | BlastProDom | 575-799 |
| Ankyrin | FPrintScan | 279-291, 464-476 |
| Ankyrin | HMMPfam | 178-211, 212-244, 245-277, 278-310, |
| | | 311-345, 346-376, 381-414, 416-450, |
| | | 451-483, 493-525 |
| Protein kinase | HMMPfam | 575-853 |
| Tyrosine protein | HMMSmart | 575-853 |
| kinase | | |
| Serine | HMMSmart | 575-857 |
| Ankyrin | HMMSmart | 178-208, 212-241, 245-274, 278-307, |
| | | 311-342, 346-377, 381-412, 416-447, |
| | | 451-480, 493-522 |
| Protein kinase | ProfileScan | 575-857 |
| Ankyrin | ProfileScan | 212-244, 245-277, 281-310, 311-343, |
| | | 381-413, 451-483 |
| Ankyrin | ProfileScan | 170-513 |
| Protein kinase | ScanRegExp | 581-602 |

Variant protein C03950_{—}3_P5 (SEQ. ID NO:212) is encoded by the following transcript(s): C03950_{—}3_T2 (SEQ. ID NO:156), for which the coding portion starts at position 3 and ends at position 2909. The transcript also has the following SNPs as listed in Table 91 (given according to their position on the nucleotide sequence, with the alternative nucleic acid listed.

TABLE 91
Nucleic acid SNPs

| SNP position(s) on nucleotide | |
|---|---|
| sequence | Alternative nucleic acid(s) |
| 263 | A -> G |
| 357 | A -> C |

Variant protein C03950_{—}3_P7 (SEQ. ID NO:213) according to the present invention is encoded by transcript C03950_{—}3_T4 (SEQ. ID NO:157). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

### 1. Comparison Report Between C03950_{—}3_P7 (SEQ. ID NO:213) and TNI3K_HUMAN (SEQ. ID NO: 396):

A. An isolated chimeric polypeptide encoding for C03950_{—}3_P7 (SEQ. ID NO:213), comprising a first amino acid sequence being at least 70%, optionally at least 80%, preferably at least 85%, more preferably at least 90% and most preferably at least 95%, homologous to a polypeptide having the sequence MGNYKSRPTQTCT (SEQ. ID NO: 346) corresponding to amino acids 1-13 of C03950_{—}3_P7 (SEQ. ID NO:213), a second amino acid sequence being at least 90% homologous to DEWKKKVSESYVITTERLEDDLQIKEKELTELRNIEGSDEAFSKVNLNYRTENGLSLLHLCCIC GGKKSHIRTLMLKGLRPSRLTRNGFTALHLAVYKDNAELITSLLHSGADIQQVGYGGLTALHI ATIAGHLEAADVLLQHGANVNIQDAVFFTPLHIAAYYGHBQVTRLLLKFGADVNVSGEVGDR PLHLASAKGFLNIAKLLMEEGSKADVNAQDNEDHVPLHFCSRFGHHDIVKYLLQSDLEVQPH VVNIYGDTPLHLACYNGKFEVAKEIIQISGTESLTKENIFSETAHISACTYGKSIDLVKFLLDQN VININHQGRDGHTGLHSACYHGHIRLVQFLLDNGADMNLVACDPSRSSGEKDEQTCLMWAY EKGHDAIVTILKHYKRTIQDELPCNEYSQPGGDGSYVSVPSPLGKIKSMTKEKADILLLRAGLP SHFHLQLSEIEFHEIIGSGSFGKVYKGRCRNKIVAIKRYRANTYCSKSDVDMFCREVSILCQLN HPCVIQFVGACLNDPSQFAIVTQYISGGSLFSLLHEQKRILDLQSKLIIAVDVAKGMEYLHNLT QPIIHRDLN corresponding to amino acids 115-691 of TNI3K_HUMAN (SEQ. ID NO: 396), which also corresponds to amino acids 14-590 of C03950_{—}3_P7 (SEQ. ID NO:213), a third amino acid sequence being at least 70%, optionally at least 80%, preferably at least 85%, more preferably at least 90% and most preferably at least 95%, homologous to a polypeptide having the sequence RSAITSRIWITHSICIWRGAHYFNREECNFRCMLTSAILK corresponding to amino acids 591-630 of C03950_{—}3_P7 (SEQ. ID NO:213), and a fourth amino acid sequence being at least 90% homologous to ESRFLQSLDEDNMTKQPGNIAWMAPEVFTQCTRYTIKADVFSYALCLWEELTGEIPFAHLKPA AAAADMAYHHERPPIGYSIPKPISSLLIRGWNACPEGRPEFSEVVIVIKLEECLCNIELMSPASSNS SGSLSPSSSSDCLVNRGGPGRSHVAALRSRFELEYALNARSYAALSQSAGQYSSQGLSLEEMK RSLQYTPIDKYGYVSDPMSSMHFHSCRNSSSFEDSS corresponding to amino acids 710-936 of TNI3K_HUMAN (SEQ. ID NO: 396), which also corresponds to amino acids 631-857 of C03950_{—}3_P7 (SEQ. ID NO:213), wherein said first amino acid sequence, second amino acid sequence, third amino acid sequence and fourth amino acid sequence are contiguous and in a sequential order.

B. An isolated polypeptide encoding for a head of C03950_{—}3_P7 (SEQ. ID NO:213), comprising a polypeptide being at least 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_T¹7 (SEQ. ID NO:213).

### 2. Comparison Report Between C03950_{—}3_P7 (SEQ. ID NO:213) and NP_{—}057062 (SEQ. ID NO:210):

A. An isolated chimeric polypeptide encoding for C03950_{—}3_P7 (SEQ. ID NO:213), comprising a first amino acid sequence being at least 90% homologous to MGNYKSRPTQTCTDEWKKKVSESYVITIERLEDDLQIKEKELTELRNIFGSDEAFSKVNLNYR TENGLSLLHLCCICGGKKSHIRTLMLKGLRPSRLTRNGFTALHLAVYKDNAELITSLLHSGADI QQVGYGGLTALHLATIAGHLEAADVLLQHGANVNIQDAVFFTPLIIIAAYYGHEQVTRLLLKF GADVNVSGEVGDRPLHLASAKGFLNIAKLLMEEGSKADVNAQDNEDHVPLHFCSRFGHEDI VKYLLQSDLEVQPHVVNIYGDTPLHLACYNGKFEVAKEIIQISGTESLTKENIFSETAFHSACT YGKSIDLVKFLLDQNVININHQGRDGHTGLHSACYHGBIRLVQFLLDNGADMNLVACDPSRS SGEKDEQTCLMWAYEKGHDAIVTLLKHYKRPQDELPCNEYSQPGGDGSYVSVPSPLGKIKSM TKEKADILLLRAGLPSHFHLQLSEIEFHEIIGSGSFGKVYKGRCRNKIVAIKRYRANTYCSKSDV DMFCREVSILCQLNFIPCVIQFVGACLNDPSQFAIVTQYISGGSLFSLLHEQKRILDLQSKLIIAV DVAKGMEYLHNLTQPIIHRDLN corresponding to amino acids 1-590 of NP_{—}057062 (SEQ. ID NO:210), which also corresponds to amino acids 1-590 of C03950_{—}3_P7 (SEQ. ID NO:213), a second amino acid sequence being at least 70%, optionally at least 80%, preferably at least 85%, more preferably at least 90% and most preferably at least 95%, homologous to a polypeptide having the sequence RSAITSRIWITHSICIWRGAHYFNREECNFRCMLTSAILK corresponding to amino acids 591-630 of C03950_{—}3_P7 (SEQ. ID NO:213), and a third amino acid sequence being at least 90% homologous to ESRFLQSLDEDNMTKQPGNLRWMAPEVFTQCTRYTIKADVFSYALCLWEILTGEIPFAHLKPA AAAADMAYHHIRPPIGYSIPKPISSLLIRGWNACPEGRPEFSEVVMKLEECLCNIELMSPASSNS SGSLSPSSSSDCLVNRGGPGRSHVAALRSRFELEYALNARSYAALSQSAGQYSSQGLSLEEMK RSLQYTPIDKYGYVSDPMSSMHFHSCRNSSSFEDSS corresponding to amino acids 609-835 of NP_{—}057062 (SEQ. ID NO:210); which also corresponds to amino acids 631-857 of C03950_{—}3_P7 (SEQ. ID NO:213), wherein said first amino acid sequence, second amino acid sequence and third amino acid sequence are contiguous and in a sequential order.

### 3. Comparison Report Between C03950_{—}3_P7 (SEQ. ID NO:213) and Q9Y2V6_HUMAN (SEQ. ID NO:210):

A. An isolated chimeric polypeptide encoding for C03950_{—}3_P7 (SEQ. ID NO:213), comprising a first amino acid sequence being at least 90% homologous to MGNYKSRPTQTCTDEWKKKVSESYVITTERLEDDLQIKEKELTELRNIFGSDEAFSKVNLNYR TENGLSLLIILCCICGGKKSHIRTLMLKGLRPSRLTRNGFTALBLAVYKDNAELITSLLHSGADI QQVGYGGLTALBIATIAGHLEAADVLLQHGANVNIQDAVFFTPLHIAAYYGHEQVTRLLLKF GADVNVSGEVGDRPLHLASAKGFLNIAKLLMEEGSKADVNAQDNEDHVPLHFCSRFGEHDI VKYLLQSDLEVQPHVVNIYGDTPLHLACYNGKFEVAKEIIQISGTESLTKENIFSETAFHSACT YGKSIDLVKFLLDQNVININHQGRDGHTGLHSACYHGHIRLVQFLLDNGADMNLVACDPSRS SGEKDEQTCLMWAYEKGHDAIVTLLKHYKRPQDELPCNEYSQPGGDGSYVSVPSPLGKIKSM TKEKADILLLRAGLPSIEFHLQLSEIEFHEIIGSGSFGKVYKGRCRNKIVAIKRYRANTYCSKSDV DMFCREVSILCQLNHPCVIQFVGACLNDPSQFAIVTQYISGGSLFSLLHEQKRILDLQSKLIIAV DVAKGMEYLHNLTQPIIHRDLN corresponding to amino acids 1-590 of Q9Y2V6_HUMAN (SEQ. ID NO:210), which also corresponds to amino acids 1-590 of C03950_{—}3_P7 (SEQ. ID NO:213), a second amino acid sequence being at least 70%, optionally at least 80%, preferably at least 85%, more preferably at least 90% and most preferably at least 95%, homologous to a polypeptide having the sequence RSAITSRIWITHSICIWRGAHYFNREECNFRCMLTSAILK corresponding to amino acids 591-630 of C03950_{—}3_P7 (SEQ. ID NO:213), and a third amino acid sequence being at least 90% homologous to ESRFLQSLDEDNMTKQPGNLRWMAPEVFTQCTRYTIKADVFSYALCLWEILTGEIPFAHLKPA AAAADMAYHEIRPPIGYSIPKPISSLLIRGWNACPEGRPEFSEVVMKLEECLCNIELMSPASSNS SGSLSPSSSSDCLVNRGGPGRSHVAALRSRFELEYALNARSYAALSQSAGQYSSQGLSLEEMK RSLQYTPIDKYGYVSDPMSSMHFHSCRNSSSFEDSS corresponding to amino acids 609-835 of Q9Y2V6_HUMAN (SEQ. ID NO:210), which also corresponds to amino acids 631-857 of C03950_{—}3_P7 (SEQ. ID NO:213), wherein said first amino acid sequence, second amino acid sequence and third amino acid sequence are contiguous and in a sequential order.

### 4. Comparison Report Between C03950_{—}3_P7 (SEQ. ID NO:213), and Q6MZS9_HUMAN (SEQ. ID NO:211):

A. An isolated chimeric polypeptide encoding for C03950_{—}3_P7 (SEQ. ID NO:213), comprising a first amino acid sequence being at least 70%, optionally at least 80%, preferably at least 85%, more preferably at least 90% and most preferably at least 95%, homologous to a polypeptide having the sequence MGNYKSRPTQTCT (SEQ. ID NO: 346) corresponding to amino acids 1-13 of C03950_{—}3_P7 (SEQ. ID NO:213), a second amino acid sequence being at least 90% homologous to DEWKKKVSESYVITIERLEDDLQIKEKELTELRNIFGSDEAFSKVNLNYRTENGLSLLHLCCIC GGKKSHIRTLMLKGLRPSRLTRNGFTALHLAVYKDNAELITSLLHSGADIQQVGYGGLTALBI ATIAGMEAADVLLQHGANVNIQDAVFFTPLHEAAYYGREQVTRLLLKFGADVNVSGEVGDR PLHLASAKGFLNIAKLLMEEGSKADVNAQDNEDHVPLHFCSRFGHHD corresponding to amino acids 132-367 of Q6MZS9_HUMAN (SEQ. ID NO:211), which also corresponds to amino acids 14-249 of C03950_{—}3_P7 (SEQ. ID NO:213), a bridging amino acid I corresponding to amino acid 250 of C03950_{—}3_P7 (SEQ. ID NO:213), a third amino acid sequence being at least 90% homologous to VKYLLQSDLEVQPHVVNIYGDTPLHLACYNGKFEVAKEIIQISGTESLTKENEFSETAFHSACT YGKSIDLVKFLLDQNVININHQGRDGHTGLHSACYHGHIRLVQFLLDNGADM corresponding to amino acids 369-484 of Q6MZS9_HUMAN (SEQ. ID NO:211), which also corresponds to amino acids 251-366 of C03950_{—}3_P7 (SEQ. ID NO:213), a bridging amino acid N corresponding to amino acid 367 of C03950_{—}3_P7 (SEQ. ID NO:213), a fourth amino acid sequence being at least 90% homologous to LVACDPSRSSGEKDEQTCLMWAYEKGBDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNHPCVIQFVGACLNDPSQFAIVTQYISGGSLFSLLHEQKRI LDLQSKLIIAVDVAKGMEYLHNLTQPIIHRDLNR corresponding to amino acids 486-709 of Q6MZS9_HUMAN (SEQ. ID NO:211), which also corresponds to amino acids 368-591 of C03950_{—}3_P7 (SEQ. ID NO:213), and a fifth amino acid sequence being at least 70%, optionally at least 80%, preferably at least 85%, more preferably at least 90% and most preferably at least 95% homologous to a polypeptide having the sequence SAITSRIWITHSICIWRGAHYFNREECNFRCMLTSAILKESRFLQSLDEDNMTKQPGNLRWMA PEVFTQCTRYTIKADVESYALCLWEILTGEIPFAHLKPAAAAADMAYHHIRPPIGYSIPKPISSL LIRGWNACPEGRPEFSEVVMKLEECLCNIELMSPASSNSSGSLSPSSSSDCLVNRGGPGRSHVA ALRSRFELEYALNARSYAALSQSAGQYSSQGLSLEEMKRSLQYTPIDKYGYVSDPMSSMHFH SCRNSSSFEDSS (SEQ. ID NO: 345) corresponding to amino acids 592-857 of C03950_{—}3_P7 (SEQ. ID NO:213), wherein said first amino acid sequence, second amino acid sequence, bridging amino acid, third amino acid sequence, bridging amino acid, fourth amino acid sequence and fifth amino acid sequence are contiguous and in a sequential order.

B. An isolated polypeptide encoding for a head of C03950_{—}3_P7 (SEQ. ID NO:213), comprising a polypeptide being at least 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P7 (SEQ. ID NO:213).

C. An isolated polypeptide encoding for an edge portion of C03950_{—}3_P7 (SEQ. ID NO:213), comprising an amino acid sequence being at least 70%, optionally at least about 80%, preferably at least about 85%, more preferably at least about 90% and most preferably at least about 95% homologous to the sequence SAITSRIWITHSICIWRGAHYFNREECNFRCMLTSAILKESRFLQSLDEDNMTKQPGNLRWMA PEVFTQCTRYTIKADVFSYALCLWEILTGEIPFAHLKPAAAAADMAYHHIRPPIGYSIPKPISSL LIRGWNACPEGRPEFSEVVMKLEECLCNIELMSPASSNSSGSLSPSSSSDCLVNRGGPGRSHVA ALRSRFELEYALNARSYAALSQSAGQYSSQGLSLEEMKRSLQYTPIDKYGYVSDPMSSMHFH SCRNSSSFEDSS (SEQ. ID NO: 345) of C03950_{—}3_P7 (SEQ. ID NO:213).

The localization of the variant protein was determined according to results from a number of different software programs and analyses, including analyses from SignalP and other specialized programs. The variant protein is believed to be located intracellularly.

The variant protein has the following domains, as determined by using InterPro. The domains are described in Table 92:

TABLE 92
InterPro domain(s)

| Domain | | |
|---|---|---|
| description | Analysis type | Position(s) on protein |
| Protein kinase | BlastProDom | 463-687 |
| Ankyrin | FPrintScan | 167-179, 352-364 |
| Ankyrin | HMMPfam | 66-99, 100-132, 133-165, 166-198, |
| | | 199-233, 234-264, 269-302, 304-338, |
| | | 339-371, 381-413 |
| Protein kinase | HMMPfam | 463-741 |
| Tyrosine protein | HMMSmart | 463-741 |
| kinase | | |
| Serine | HMMSmart | 463-745 |
| Ankyrin | HMMSmart | 66-96, 100-129, 133-162, 166-195, |
| | | 199-230, 234-265, 269-300, 304-335, |
| | | 339-368, 381-410 |
| Protein kinase | ProfileScan | 463-745 |
| Ankyrin | ProfileScan | 100-132, 133-165, 169-198, 199-231, |
| | | 269-301, 339-371 |
| Ankyrin | ProfileScan | 58-401 |
| Protein kinase | ScanRegExp | 469-490 |

Variant protein C03950_{—}3_P7 (SEQ. ID NO:213) is encoded by the following transcript(s): C03950_{—}3_T4 (SEQ. ID NO:157), for which the coding portion starts at position 389 and ends at position 2959.

Variant protein C03950_{—}3_P9 (SEQ. ID NO:214) according to the present invention is encoded by transcript C03950_{—}3_T7 (SEQ. ID NO:158). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

### 1. Comparison Report Between C03950_{—}33³9 (SEQ. ID NO:214) and TNI3K_HUMAN (SEQ. ID NO: 396):

A. An isolated chimeric polypeptide encoding for C03950_{—}3_P9 (SEQ. ID NO:214), comprising a first amino acid sequence being at least 70%, optionally at least 80%, preferably at least 85%, more preferably at least 90% and most preferably at least 95%, homologous to a polypeptide having the sequence MGNYKSRPTQTCT (SEQ. ID NO: 346) corresponding to amino acids 1-13 of C03950_{—}3_P9 (SEQ. ID NO:214), a second amino acid sequence being at least 90% homologous to DEWKKKVSESYVITIERLEDDLQIKEKELTELRNIEGSDEAFSKVNLNYRTENGLSLUILCCIC GGKKSHIRTLMIKGLRPSRLTRNGFTALBLAVYKDNAELITSLLHSGADIQQVGYGGLTALHI ATIAGHLEAADVLLQHGANVNIQDAVFFTPLHIAAYYGHEQVIRLLLKFGADVNVSGEVGDR PLIILASAKGFLNIAKLLMEEGSKADVNAQDNEDHVPLHFCSRFGHHDIVKYLLQSDLEVQPH VVNIYGDTPLHLACYNGKFEVAKEIEQISGTESLTKENIFSETAFHSACTYGKSIDLVKFLLDQN VININHQGRDGHTGLHSACYHGBIRLVQFLLDNGADMNLVACDPSRSSGEKDEQTCLMWAY EKGBDAIVTLLKHYKRPQDELPCNEYSQPGGDGSYVSVPSPLGKIKSMTKEKADILLLRAGLP SHFHLQLSEIEFHEUGSGSFGKVYKGRCRNKIVAIKRYRANTYCSKSDVDMFCREVSILCQLN HPCVIQFVGACLNDPSQFAIVTQYISGGSLFSLLHEQKRILDLQSKLIIAVDVAKGMEYLHNLT QPIIHRDLNSFINILLYEDGHAVVADFGESRFLQSLDEDNMTKQPGNLRWMAPEVFTQCTRYTI KADVFSYALCLWEILTGEIPFAHLKPAAAAADMAYBBIRPPIGYSIPKPISSLLIRGWNACPEG RPEFSEVVMKLEECLCNTELMSPASSNSSGSLSPSSSSDCLVNRGGPGRSHVAALRSRFELEYA LNARSYAALSQSAGQYSSQGLSLEEMKRSLQYTPIDKY corresponding to amino acids 115-911 of TNI3K_HUMAN (SEQ. ID NO: 396), which also corresponds to amino acids 14-810 of C03950_{—}3_P9 (SEQ. ID NO:214), and a third amino acid sequence being at least 70%, optionally at least 80%, preferably at least 85%, more preferably at least 90% and most preferably at least 95% homologous to a polypeptide having the sequence DVTS (SEQ. ID NO: 349) corresponding to amino acids 811-814 of C03950_{—}3P9 (SEQ. ID NO:214), wherein said first amino acid sequence, second amino acid sequence and third amino acid sequence are contiguous and in a sequential order.

B. An isolated polypeptide encoding for a head of C03950_{—}3_P9 (SEQ. ID NO:214), comprising a polypeptide being at least 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P9 (SEQ. ID NO:214).

C. An isolated polypeptide encoding for an edge portion of C03950_{—}3_P9 (SEQ. ID NO:214), comprising an amino acid sequence being at least 70%, optionally at least about 80%, preferably at least about 85%, more preferably at least about 90% and most preferably at least about 95% homologous to the sequence DVTS (SEQ. ID NO: 349) of C03950_{—}3_P9 (SEQ. ID NO:214).

### 2. Comparison Report Between C03950_{—}3_P9 (SEQ. ID NO:214) and NP_{—}057062 (SEQ. ID NO:210):

A. An isolated chimeric polypeptide encoding for C03950_{—}3_P9 (SEQ. ID NO:214), comprising a first amino acid sequence being at least 90% homologous to MGNYKSRPTQTCTDEWKKKVSESYVITIERLEDDLQIKEKELTELRNIFGSDEAFSKVNLNYR TENGLSLLHLCCICGGKKSHIRTLMLKGLRPSRLTRNGFTALHLAVYKDNAELITSLLHSGADI QQVGYGGLTALHIATIAGHLEAADVLLQHGANVNIQDAVFFTPLHIAAYYGREQVTRLLLKF GADVNVSGEVGDRPLBLASAKGFLNIAKLLMEEEGSKADVNAQDNEDHVPLHFCSREGUHDI VKYLLQSDLEVQPHVVNIYGDTPLBLACYNGKFEVAKEIIQISGTESLTKENIFSETAFHSACT YGKSIDLVKFLLDQNVININHQGRDGHTGLHSACYHGHERLVQFLLDNGADMNLVACDPSRS SGEKDEQTCLMWAYEKGBDAIVTLLKHYKRPQDELPCNEYSQPGGDGSYVSVPSPLGKIKSM TKEKADILLLRAGLPSHFHLQLSEIEFHEIIGSGSFGKVYKGRCRNKIVAIKRYRANTYCSKSDV DMFCREVSILCQLNIIPCVIQFVGACLNDPSQFAIVTQYISGGSLFSLLHEQKRELDLQSKLIIAV DVAKGMEYLBNLTQPIEHRDLNSIINILLYEDGHAVVADFGESRFLQSLDEDNMTKQPGNLR WMAPEVETQCTRYTIKADVESYALCLWEILTGEIPFAHLKPAAAAADMAYHHIRPPIGYSIPKP ISSLLIRGWNACPEGRPEFSEVVMKLEECLCNIELMSPASSNSSGSLSPSSSSDCLVNRGGPGRS HVAALRSRFELEYALNARSYAALSQSAGQYSSQGLSLEEMKRSLQYTPIDKY corresponding to amino acids 1-810 of NP_{—}057062 (SEQ. ID NO:210), which also corresponds to amino acids 1-810 of C03950_{—}3_P9 (SEQ. ID NO:214), and a second amino acid sequence being at least 70%, optionally at least 80%, preferably at least 85%, more preferably at least 90% and most preferably at least 95% homologous to a polypeptide having the sequence DVTS (SEQ. ID NO: 349) corresponding to amino acids 811-814 of C03950_{—}3_P9 (SEQ. ID NO:214), wherein said first amino acid sequence and second amino acid sequence are contiguous and in a sequential order.

### 3. Comparison Report Between C03950_{—}3_P9 (SEQ. ID NO:214) and Q9Y2V6_HUMAN (SEQ. ID NO:210):

A. An isolated chimeric polypeptide encoding for C03950_{—}3_P9 (SEQ. ID NO:214), comprising a first amino acid sequence being at least 90% homologous to MGNYKSRPTQTCTDEWKKKVSESYVITIERLEDDLQIKEKELTELRNIFGSDEAFSKVNLNYR TENGLSLLHLCCICGGKKSHIRTLMILKGLRPSRLTRNGFTALHLAVYKDNAELITSLLHSGADI QQVGYGGLTALHIATIAGHLEAADVLLQHGANVNIQDAVFFTPLBIAAYYGHEQVTRLLLKF GADVNVSGEVGDRPLHLASAKGFLNIAKLLMEEGSKADVNAQDNEDHVPLHFCSRFGHHDI VKYLLQSDLEVQPHVVNTYGDTPLHLACYNGKFEVAKEIIQISGTESLTKENIFSETAFHSACT YGKSIDLVKFLLDQNVININHQGRDGHTGLHSACYHGHIRLVQFLLDNGADMNLVACDPSRS SGEKDEQTCLMWAYEKGHDAIVTLLKHYKRPQDELPCNEYSQPGGDGSYVSVPSPLGKIKSM TKEKADILLLRAGLPSHFHLQLSEMEHEIIGSGSFGKVYKGRCRNKIVAIKRYRANTYCSKSDV DMFCREVSILCQLNHPCVIQFVGACLNDPSQFAIVTQYISGGSLFSLLHEQKRILDLQSKLIIAV DVAKGMEYLHNLTQPIIHRDLNSHNILLYEDGHAVVADFGESRFLQSLDEDNMTKQPGNLR WMAPEVFTQCTRYTIKADVFSYALCLWEILTGEIPFAHLKPAAAAADMAYHHIRPPIGYSIPKP ISSLLIRGWNACPEGRPEFSEVVMKLEECLCNIELMSPASSNSSGSLSPSSSSDCLVNRGGPGRS HVAALRSRFELEYALNARSYAALSQSAGQYSSQGLSLEEMKRSLQYTPIDKY corresponding to amino acids 1-810 of Q9Y2V6_HUMAN (SEQ. ID NO:210), which also corresponds to amino acids 1-810 of C03950_{—}339 (SEQ. ID NO:214), and a second amino acid sequence being at least 70%, optionally at least 80%, preferably at least 85%, more preferably at least 90% and most preferably at least 95% homologous to a polypeptide having the sequence DVTS (SEQ. ID NO: 349) corresponding to amino acids 811-814 of C03950_{—}3_P9 (SEQ. ID NO:214), wherein said first amino acid sequence and second amino acid sequence are contiguous and in a sequential order.

B. An isolated polypeptide encoding for an edge portion of C03950_{—}3_P9 (SEQ. ID NO:214), comprising an amino acid sequence being at least 70%, optionally at least about 80%, preferably at least about 85%, more preferably at least about 90% and most preferably at least about 95% homologous to the sequence DVTS (SEQ. ID NO: 349) of C03950_{—}3_P9 (SEQ. ID NO:214).

### 4. Comparison Report Between C03950_{—}3_P9 (SEQ. ID NO:214) and Q6MZS9_HUMAN (SEQ. ID NO:211):

A. An isolated chimeric polypeptide encoding for C03950_{—}3_P9 (SEQ. ID NO:214), comprising a first amino acid sequence being at least 70%, optionally at least 80%, preferably at least 85%, more preferably at least 90% and most preferably at least 95%, homologous to a polypeptide having the sequence MGNYKSRPTQTCT (SEQ. ID NO: 346) corresponding to amino acids 1-13 of C03950_{—}3_P9 (SEQ. ID NO:214), a second amino acid sequence being at least 90% homologous to DEWKKKVSESYVITIERLEDDLQIKEKELTELRNIFGSDEAFSKVNLNYRTENGLSLUILCCIC GGKKSHIRTLMLKGLRPSRLTRNGFTALBLAVYKDNAELITSLLHSGADIQQVGYGGLTALHI ATJAGHLEAADVLLQHGANVNIQDAVFFTPLBIAAYYGHEQVTRLLLKFGADVNVSGEVGDR PLHLASAKGFLNIAKLLMEEGSKADVNAQDNEDHVPLHFCSRFGHHD corresponding to amino acids 132-367 of Q6MZS9_HUMAN (SEQ. ID NO:211), which also corresponds to amino acids 14-249 of C03950_{—}3_P9 (SEQ. ID NO:214), a bridging amino acid I corresponding to amino acid 250 of C03950_{—}3_P9 (SEQ. ID NO:214), a third amino acid sequence being at least 90% homologous to VKYLLQSDLEVQPHVVNTYGDTPUILACYNGKFEVAKEIIQISGTESLTKENIFSETAFHSACT YGKSIDLVKFLLDQNVININHQGRDGHTGLHSACYHGHIRLVQFLLDNGADM corresponding to amino acids 369-484 of Q6MZS9_HUMAN (SEQ. ID NO:211), which also corresponds to amino acids 251-366 of C03950_{—}3_P9 (SEQ. ID NO:214), a bridging amino acid N corresponding to amino acid 367. of C03950_{—}3_P9 (SEQ. ID NO:214), a fourth amino acid sequence being at least 90% homologous to LVACDPSRSSGEKDEQTCLMWAYEKGHDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNHPCVIQFVGACLNDPSQFAIVTQYISGGSLFSLLHEQKRI LDLQSKLIIAVDVAKGMEYLHNLTQPIIHRDLN corresponding to amino acids 486-708 of Q6MZS9_HUMAN (SEQ. ID NO:211), which also corresponds to amino acids 368-590 of C03950_{—}3_P9 (SEQ. ID NO:214), and a fifth amino acid sequence being at least 70%, optionally at least 80%, preferably at least 85%, more preferably at least 90% and most preferably at least 95% homologous to a polypeptide having the sequence SHNILLYEDGHAVVADFGESRFLQSLDEDNMTKQPGNLRWMAPEVFTQCTRYTIKADVFSYA LCLWEELTGEIPFAHLKPAAAAADMAYHHIRPPIGYSIPKPISSLLIRGWNACPEGRPEFSEVVM KLEECLCNIELMSPASSNSSGSLSPSSSSDCLVNRGGPGRSHVAALRSRFELEYALNARSYAAL SQSAGQYSSQGLSLEEMKRSLQYTPIDKYDVTS (SEQ. ID NO: 350) corresponding to amino acids 591-814 of C03950_{—}3_P9 (SEQ. ID NO:214), wherein said first amino acid sequence, second amino acid sequence, bridging amino acid, third amino acid sequence, bridging amino acid, fourth amino acid sequence and fifth amino acid sequence are contiguous and in a sequential order.

B. An isolated polypeptide encoding for a head of C03950_{—}3_P9 (SEQ. ID NO:214), comprising a polypeptide being at least 70%, optionally at least about 80%, preferably at least about 85%, more preferably at least about 90% and most preferably at least about 95% homologous to the sequence MGNYKSRPTQTCT (SEQ. ID NO: 346) of C03950_{—}3P9 (SEQ. ID NO:214).

C. An isolated polypeptide encoding for an edge portion of C03950_{—}3_P9 (SEQ. ID NO:214), comprising an amino acid sequence being at least 70%, oₚtionally at least about 80%, preferably at least about 85%, more preferably at least about 90% and most preferably at least about 95% homologous to the sequence SHNILLYEDGHAVVADFGESRFLQSLDEDNMTKQPGNLRWMAPEVFTQCTRYTIKADVFSYA LCLWELLTGEIPFAHLKPAAAAADMAYHHIRPPIGYSIPKPISSLIIRGWNACPEGRPEFSEVVM KLEECLCNIELMSPASSNSSGSLSPSSSSDCLVNRGGPGRSHVAALRSRFELEYALNARSYAAL SQSAGQYSSQGLSLEEMKRSLQYTPIDKYDVTS (SEQ. ID NO: 350) of C03950_{—}3_P9 (SEQ. ID NO:214).

The localization of the variant protein was determined according to results from a number of different software programs and analyses, including analyses from SignalP and other specialized programs. The variant protein is believed to be located intracellularly.

The variant protein has the following domains, as determined by using InterPro. The domains are described in Table 93:

TABLE 93
InterPro domain(s)

| Domain | | |
|---|---|---|
| description | Analysis type | Position(s) on protein |
| Protein kinase | BlastProDom | 463-665 |
| Tyrosine protein | FPrintScan | 539-552, 578-596, 626-636, 690-712 |
| kinase | | |
| Ankyrin | FPrintScan | 167-179, 352-364 |
| Ankyrin | HMMPfam | 66-99, 100-132, 133-165, 166-198, |
| | | 199-233, 234-264, 269-302, 304-338, |
| | | 339-371, 381-413 |
| Protein kinase | HMMPfam | 463-719 |
| Tyrosine protein | HMMSmart | 463-719 |
| kinase | | |
| Serine | HMMSmart | 463-723 |
| Ankyrin | HMMSmart | 66-96, 100-129, 133-162, 166-195, |
| | | 199-230, 234-265, 269-300, 304-335, |
| | | 339-368, 381-410 |
| Protein kinase | ProfileScan | 463-723 |
| Ankyrin | ProfileScan | 100-132, 133-165, 169-198, 199-231, |
| | | 269-301, 339-371 |
| Ankyrin | ProfileScan | 58-401 |
| Protein kinase | ScanRegExp | 469-490 |

Variant protein C03950_{—}3_P9 (SEQ. ID NO:214) is encoded by the following transcript(s): C03950_{—}3_T7 (SEQ. ID NO:158), for which the coding portion starts at position 389 and ends at position 2830.

Variant protein C03950_{—}3_P10 (SEQ. ID NO:215) according to the present invention is encoded by transcript C03950_{—}3_T8 (SEQ. ID NO:159). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

### 1. Comparison Report Between C03950_{—}3_P10 (SEQ. ID NO:215) and TNI3K_HUMAN (SEQ. ID NO: 396):

A. An isolated chimeric polypeptide encoding for C03950_{—}3P10 (SEQ. ID NO:215), comprising a first amino acid sequence being at least 70%, optionally at least 80%, preferably at least 85%, more preferably at least 90% and most preferably at least 95%, homologous to a polypeptide having the sequence PSPCGLHFLIPWLTQ (SEQ. ID NO: 351) corresponding to amino acids 1-15 of C03950_{—}3_P10 (SEQ. ID NO:215), and a second amino acid sequence being at least 90% homologous to DNAELITSLLHSGADIQQVGYGGLTALHIATIAGHLEAADVLLQHGANVNIQDAVFFTPLHIA AYYGHEQVTRLLLKFGADVNVSGEVGDRPLHLASAKGFLNIAKLLMEEGSKADVNAQDNED HVPLHFCSRFGHHDIVKYLLQSDLEVQPHVVNIYGDTPLHLACYNGKFEVAKEIIQISGTESLT KENIFSETAPHSACTYGKSIDLVKFLLDQNVININHQGRDGHTGLHSACYHGHIRLVQFLLDN GADMNLVACDPSRSSGEKDEQTCLMWAYEKGFIDANTLLKHYKRPQDELPCNEYSQPGGD GSYVSVPSPLGKIKSMTKEKADILLLRAGLPSHFHLQLSEIEFHEIIGSGSFGKVYKGRCRNKIV ALKRYRANTYCSKSDVDMCFCREVSILCQLNITPCVIQFVGACLNDPSQFAIVTQYISGGSLFSLL HEQKRILDLQSKLBAVDVAKGMEYLHNLTQPBEHRDLNSHNILLYEDGHAVVADFGESRFLQS LDEDNMTKQPGNLRWMAPEVFTQCTRYTIKADVFSYALCLWEILTGEIPFAHLKPAAAAAD MAYHHIRPPIGYSEPKPISSLLIRGWNACPEGRPEFSEVVIAKLEECLCNIELMSPASSNSSGSLSP SSSSDCLVNIZGGPGRSHVAALRSRFELEYALNARSYAALSQSAGQYSSQGLSLEEMXRSLQY TPIDKYGYVSDPMSSMHFHSCRNSSSFEDSS corresponding to amino acids 213-936 of TNI3K_HUMAN (SEQ. ID NO: 396), which also corresponds to amino acids 16-739 of C03950_{—}3_P10 (SEQ. ID NO:215), wherein said first amino acid sequence and second amino acid sequence are contiguous and in a sequential order.

B. An isolated polypeptide encoding for a head of C03950_{—}3_P10 (SEQ. ID NO:215), comprising a polypeptide being at least 70%, optionally at least about 80%, preferably at least about 85%, more preferably at least about 90% and most preferably at least about 95% homologous to the sequence PSPCGLHFLIPWLTQ (SEQ. ID NO: 351) of C03950_{—}3_P10 (SEQ. ID NO:215).

### 2. Comparison Report Between C03950_{—}3_P10 (SEQ. ID NO:215) and Q6MZS9_HUMAN (SEQ NO:211):

A. An isolated chimeric polypeptide encoding for C03950_{—}3_P10 (SEQ. ID NO:215), comprising a first amino acid sequence being at least 70%, optionally at least 80%, preferably at least 85%, more preferably at least 90% and most preferably at least 95%, homologous to a polypeptide having the sequence PSPCGLHFLIPWLTQ (SEQ. ID NO: 351) corresponding to amino acids 1-15 of C03950_{—}3_P10 (SEQ. ID NO:215), a second amino acid sequence being at least 90% homologous to DNAELITSLLHSGADIQQVGYGGLTALHIATIAGHLEAADVLLQHGANVNIQDAVFFTPLHIA AYYGREQVTRLLLKFGADVNVSGEVGDRPLHLASAKGFLNIAKLLMEEGSKADVNAQDNED HVPLHFCSRFGHHD corresponding to amino acids 230-367 of Q6MZS9_HUMAN (SEQ. ID NO:211), which also corresponds to amino acids 16-153 of C03950_{—}3_P10 (SEQ. ID NO:215), bridging amino acid I corresponding to amino acid 154 of C03950_{—}3_P10 (SEQ. ID NO:215), a third amino acid sequence being at least 90% homologous to VKYLLQSDLEVQPHVVNIYGDTPLHLACYNGKFEVAKEIIQISGTESLTKENIFSETAFHSACT YGKSEDLVKFLLDQNVININHQGRDGHTGLHSACYHGHIRLVQFLLDNGADM corresponding to amino acids 369-484 of Q6MZS9_HUMAN (SEQ. ID NO:211), which also corresponds to amino acids 155-270 of C03950_{—}3P10 (SEQ. ID NO:215), a bridging amino acid N corresponding to amino acid 271 of C03950_{—}3_P10 (SEQ. ID NO:215), a fourth amino acid sequence being at least 90% homologous to LVACDPSRSSGEKDEQTCLMWAYEKGHDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNHPCVIQFVGACLNDPSQFAIVTQYISGGSLFSLLHEQKRI LDLQSKLIIAVDVAKGMEYLHNLTQPIIHRDLN corresponding to amino acids 486-708 of Q6MZS9_HUMAN (SEQ. ID NO:211), which also corresponds to amino acids 272-494 of C03950_{—}3_P10 (SEQ. ID NO:215), and a fifth amino acid sequence being at least 70%, optionally at least 80%, preferably at least 85%, more preferably at least 90% and most preferably at least 95% homologous to a polypeptide having the sequence SHNILLYEDGHAVVADFGESRFLQSLDEDNMTKQPGNLRWMAPEVFTQCTRYTIKADVFSYA LCLWEILTGELPFAHLKPAAAAADMAYHHIRPPIGYSIPKPISSLLIRGWNACPEGRPEFSEVVM KLEECLCNIELMSPASSNSSGSLSPSSSSDCLVNRGGPGRSHVAALRSRFELEYALNARSYAAL SQSAGQYSSQGLSLEEMKRSLUTPIDKYGYVSDPMSSMEFHSCRNSS(SEQ. ID NO: 352) corresponding to amino acids 495-739 of C03950_{—}3_P10 (SEQ. ID NO:215), wherein said first amino acid sequence, second amino acid sequence, bridging amino acid, third amino acid sequence, bridging amino acid, fourth amino acid sequence and fifth amino acid sequence are contiguous and in a sequential order.

C. An isolated polypeptide encoding for an edge portion of C03950_{—}3_P10 (SEQ. ID NO:215), comprising an amino acid sequence being at least 70%, optionally at least about 80%, preferably at least about 85%, more preferably at least about 90% and most preferably at least about 95% homologous to the sequence SHNILLYEDGHAVVADFGESRFLQSLDEDNMTKQPGNLRWMAPEVFTQCTRYTIKADVFSYA LCLWEILTGEIPFAHLKPAAAAADMAYHHIRPPIGYSEFKPISSLLIRGWNACPEGRPEFSEVVM KLEECLCNIELMSPASSNSSGSLSPSSSSDCLVNRGGPGRSHVAALRSRFELEYALNARSYAAL SQSAGQYSSQGLSLEEMKRSLQYTPIDKYGYVSDPMSSMHFHSCRNSS(SEQ. ID NO: 352) of C03950_{—}3_P10 (SEQ. ID NO:215).

The localization of the variant protein was determined according to results from a number of different software programs and analyses, including analyses from SignalP and other specialized programs. The variant protein is believed to be located intracellularly.

The variant protein has the following domains, as determined by using InterPro. The domains are described in Table 94:

TABLE 94
InterPro domain(s)

| Domain | | |
|---|---|---|
| description | Analysis type | Position(s) on protein |
| Protein kinase | BlastProDom | 367-569 |
| Tyrosine protein | FPrintScan | 443-456, 482-500, 530-540, 594-616 |
| kinase | | |
| Ankyrin | FPrintScan | 71-83, 256-268 |
| Ankyrin | HMMPfam | 37-69, 70-102, 103-137, 138-168, |
| | | 173-206, 208-242, 243-275, 285-317 |
| Protein kinase | HMMPfam | 367-623 |
| Tyrosine protein | HMMSmart | 367-623 |
| kinase | | |
| Serine | HMMSmart | 367-627 |
| Ankyrin | HMMSmart | 37-66, 70-99, 103-134, 138-169, |
| | | 173-204, 208-239, 243-272, 285-314 |
| Protein kinase | ProfileScan | 367-627 |
| Ankyrin | ProfileScan | 37-69, 73-102, 103-135, 173-205, |
| | | 243 -275 |
| Ankyrin | ProfileScan | 17-305 |
| Protein kinase | ScanRegExp | 373-394 |

Variant protein C03950_{—}3_P10 (SEQ. ID NO:215) is encoded by the following transcript(s): C03950_{—}3_T8 (SEQ. ID NO:159), for which the coding portion starts at position 1 and ends at position 2217.

Variant protein C03950_{—}3_P11 (SEQ. ID NO:216) according to the present invention is encoded by transcript C03950_{—}3_T9 (SEQ. ID NO:160). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

### 1. Comparison Report Between C03950_{—}3_P11 (SEQ. ID NO:216) and TNL3K_HUMAN (SEQ. ID NO: 396):

A. An isolated chimeric polypeptide encoding for C03950_{—}3_P11 (SEQ. ID NO:216), comprising a first amino acid sequence being at least 70%, optionally at least 80%, preferably at least 85%, more preferably at least 90% and most preferably at least 95%, homologous to a polypeptide having the sequence PSPCGLHFLIPWLTQ (SEQ. ID NO: 351) corresponding to amino acids 1-15 of C03950_{—}3_P11 (SEQ. ID NO:216), a second amino acid sequence being at least 90% homologous to DNAELITSLLHSGADIQQVGYGGLTALHIATIAGHLEAADVLLQHGANVNIQDAVFFTPLHIA AYYGHEQVTRILLKFGADVNVSGEVGDRPLITLASAKGFLNIAKLLMEEGSKADVNAQDNED HVPLHFCSRFGHHDIVKYLLQSDLEVQPHVVNIYGDTPLHLACYNGKFEVAKEIEQISGTESLT KENIFSETAFHSACTYGKSIDLVKFLLDQNVININHQGRDGHTGLHSACYHGHIRLVQFLLDN GADMNLVACDPSRSSGEKDEQTCLMWAYEKGBDAIVTLLKHYKRPQDELPCNEYSQPGGD GSYVSVPSPLGKIKSMTKEKADILLLRAGLPSHFHLQLSEIEFHEIIGSGSFGKVYKGRCRNKIV AIKRYRANTYCSKSDVDMFCREVSILCQLNHPCVIQFVGACLNDPSQFAIVTQYISGGSLFSLL HEQKRILDLQSKLIIAVDVAKGMEYLHNLTQPIIHRDLN corresponding to amino acids 213-691 of TNI3K_HUMAN (SEQ. ID NO: 396), which also corresponds to amino acids 16-494 of C03950_{—}3_P11 (SEQ. ID NO:216), a third amino acid sequence being at least 70%, optionally at least 80%, preferably at least 85%, more preferably at least 90% and most preferably at least 95%, homologous to a polypeptide having the sequence RSAITSRIWITHSICIWRGAHYFNREECNFRCMLTSAILK corresponding to amino acids 495-534 of C03950_{—}3_P11 (SEQ. ID NO:216), and a fourth amino acid sequence being at least 90% homologous to ESRFLQSLDEDNMTKQPGNLRWMAPEVFTQCTRYTIKADVFSYALCLWEILTGEIPFAHLKPA AAAADMAYHHIRPPIGYSEPKPISSLLIRGWNACPEGRPEFSEVVMKLEECLCNIELMSPASSNS SGSLSPSSSSDCLVNRGGPGRSHVAALRSRFELEYALNARSYAALSQSAGQYSSQGLSLEEMK RSLQYTPIDKYGYVSDPMSSMHFHSCRNSSSFEDSS corresponding to amino acids 710-936 of TNI3K_HUMAN (SEQ. ID NO: 396), which also corresponds to amino acids 535-761 of C03950_{—}3_P11 (SEQ. ID NO:216), wherein said first amino acid sequence, second amino acid sequence, third amino acid sequence and fourth amino acid sequence are contiguous and in a sequential order.

B. An isolated polypeptide encoding for a head of C03950_{—}3_P11 (SEQ. ID NO:216), comprising a polypeptide being at least 70%, optionally at least about 80%, preferably at least about 85%, more preferably at least about 90% and most preferably at least about 95% homologous to the sequence PSPCGLHFLIPWLTQ (SEQ. ID NO: 351) of C03950_{—}3_P11 (SEQ. ID NO:216).

### 2. Comparison Report Between C03950_{—}3_P11 (SEQ. ID NO:216) and Q6MZS9_HUMAN (SEQ. ID NO:211):

A. An isolated chimeric polypeptide encoding for C03950_{—}3_P11 (SEQ. ID NO:216), comprising a first amino acid sequence being at least 70%, optionally at least 80%, preferably at least 85%, more preferably at least 90% and most preferably at least 95%, homologous to a polypeptide having the sequence PSPCGLHFLIPWLTQ (SEQ. ID NO: 351) corresponding to amino acids 1-15 of C03950_{—}3_P11 (SEQ. ID NO:216), a second amino acid sequence being at least 90% homologous to DNAELITSLLHSGADIQQVGYGGLTALHIATIAGHLENADVLLQHGANVNIQDAVFFTPLHIA AYYGHEQVTRLLLKFGADVNVSGEVGDRPLHLASAKGFLNIAKLLMEEGSKADVNAQDNED HVPLHFCSRFGHHD corresponding to amino acids 230-367 of Q6MZS9_HUMAN (SEQ. ID NO:211), which also corresponds to amino acids 16-153 of C03950_{—}3_P11 (SEQ. ID NO:216), a bridging amino acid I corresponding to amino acid 154 of C03950_{—}3_P11 (SEQ. ID NO:216), a third amino acid sequence being at least 90% homologous to VKYLLQSDLEVQPHVVNIYGDTPLHLACYNGKFEVAKEIIQISGTESLTKENIFSETAFHSACT YGKSIDLVKFLLDQNVININHQGRDGHTGLHSACYHGHIRLVQFLLDNGADM corresponding to amino acids 369-484 of Q6MZS9_HUMAN (SEQ. ID NO:211), which also corresponds to amino acids 155-270 of C03950_{—}3_P11 (SEQ. ID NO:216), a bridging amino acid N corresponding to amino acid 271 of C03950_{—}3_P11 (SEQ. ID NO:216), a fourth amino acid sequence being at least 90% homologous to LVACDPSRSSGEKDEQTCLMWAYEKGHDAIVILLKHYKRPQDELPCNEYSQPGGDGSYVSV PSPLGIUKSMTKEKADILLLRAGLPSHFHLQLSEIEFHEIIGSGSFGKVYKGRCRNKIVAIKRYR ANTYCSKSDVDMFCREVSILCQLNIIPCVIQFVGACLNDPSQFANTQYISGGSLFSLLHEQKRI LDLQSKLIIAVDVAKGMEYLHNLTQPITIARDLNR corresponding to amino acids 486-709 of Q6MZS9_HUMAN (SEQ. ID NO:211), which also corresponds to amino acids 272-495 of C03950_{—}3_P11 (SEQ. ID NO:216), and a fifth amino acid sequence being at least 70%, optionally at least 80%, preferably at least 85%, more preferably at least 90% and most preferably at least 95% homologous to a polypeptide having the sequence SAITSRIWITHSICIWRGAHYFNREECNFRCMLTSAILKESRFLQSLDEDNMTKQPGNLRWMA PEVFTQCTRYTIKADVFSYALCLWEILTGEIPFAHLKPAAAAADMAYHHIRPPIGYSIPKPISSL LIRGWNACPEGRPEFSEVVMKLEECLCNIELMSPASSNSSGSLSPSSSSDCLVNRGGPGRSHVA ALRSRFELEYALNARSYAALSQSAGQYSSQGLSLEEMKRSLQYTPIDKYGYVSDPMSSMHFH SCRNSSSFEDSS (SEQ. ID NO: 345) corresponding to amino acids 496-761 of C03950_{—}3_P11 (SEQ. ID NO:216), wherein said first amino acid sequence, second amino acid sequence, bridging amino acid, third amino acid sequence, bridging amino acid, fourth amino acid sequence and fifth amino acid sequence are contiguous and in a sequential order.

B. An isolated polypeptide encoding for a head of C03950_{—}3_P11 (SEQ. ID NO:216), comprising a polypeptide being at least 70%, optionally at least about 80%, preferably at least about 85%, more preferably at least about 90% and most preferably at least about 95% homologous to the sequence PSPCGLHFLIPVVLTQ (SEQ. ID NO: 351) of C03950_{—}3_P11 (SEQ. ID NO:216).

C. An isolated polypeptide encoding for an edge portion of C03950_{—}3 P11 (SEQ. ID NO:216), comprising an amino acid sequence being at least 70%, optionally at least about 80%, preferably at least about 85%, more preferably at least about 90% and most preferably at least about 95% homologous to the sequence SAITSRIWITHSICIWRGAHYFNREECNFRCMLTSAILKESRFLQSLDEDNMTKQPGNLRWMA PEVFTQCTRYTHCADVESYALCLWEILTGEIPFAHLKPAAAAADMAYHFIIRPPIGYSIPKPISSL LIRGWNACPEGRPEFSEVVMKLEECLCNIELMSPASSNSSGSLSPSSSSDCLVNRGGPGRSHVA ALRSRFELEYALNARSYAALSQSAGQYSSQGLSLEEMKRSLQYTPlDKYGYVSDPMSSMHFH SCRNSSSFEDSS (SEQ. ID NO: 345) of C03950_{—}3_P11 (SEQ. ID NO:216).

The localization of the variant protein was determined according to results from a number of different software programs and analyses, including analyses from SignalP and other specialized programs. The variant protein is believed to be located intracellularly.

The variant protein has the following domains, as determined by using InterPro. The domains are described in Table 95:

TABLE 95
InterPro domain(s)

| Domain | | |
|---|---|---|
| description | Analysis type | Position(s) on protein |
| Protein kinase | BlastProDom | 367-591 |
| Ankyrin | FPrintScan | 71-83, 256-268 |
| Ankyrin | HMMPfam | 37-69, 70-102, 103-137, 138-168, |
| | | 173-206, 208-242, 243-275, 285-317 |
| Protein kinase | HMMPfam | 367-645 |
| Tyrosine protein | HMMSmart | 367-645 |
| kinase | | |
| Serine | HMMSmart | 367-649 |
| Ankyrin | HMMSmart | 37-66, 70-99, 103-134, 138-169, |
| | | 173-204, 208-239, 243-272, 285-314 |
| Protein kinase | ProfileScan | 367-649 |
| Ankyrin | ProfileScan | 37-69, 73-102, 103-135, 173-205, |
| | | 243-275 |
| Ankyrin | ProfileScan | 17-305 |
| Protein kinase | ScanRegExp | 373-394 |

Variant protein C03950_{—}3_P11 (SEQ. ID NO:216) is encoded by the following transcript(s): C03950_{—}3_T9 (SEQ. ID NO:160), for which the coding portion starts at position 1 and ends at position 2283.

Variant protein C03950_{—}3_P12 (SEQ. ID NO:217) according to the present invention is encoded by transcript C03950_{—}3_T10 (SEQ. ID NO:161). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

### 1. Comparison Report Between C03950_{—}3_P12 (SEQ. ID NO:217) and TNI3K_HUMAN (SEQ. ID NO: 396):

A. An isolated chimeric polypeptide encoding for C03950_{—}3312 (SEQ. ID NO:217), comprising a first amino acid sequence being at least 70%, optionally at least 80%, preferably at least 85%, more preferably at least 90% and most preferably at least 95%, homologous to a polypeptide having the sequence AVRRGLREGGA (SEQ. ID NO: 342) corresponding to amino acids 1-11 of C03950_{—}3_P12 (SEQ. ID NO:217), a second amino acid sequence being at least 90% homologous to MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHSDEWKKKVSES YVITIERLEDDLQIKEKELTELRNIFGSDEAFSKVNLNYRTENGLSLLBLCCICGGKKSHERTLM LKGLRPSRLTRNGFTALHLAVYKDNAELITSLLHSGADIQQVGYGGLTALHIATIAGHLEAAD VLLQHGANVNIQDAVFFTPLHIAAYYGHEQVTRLLLKFGADVNVSGEVGDRPLFELASAKGFL NIAKLLMEEGSKADVNAQDNEDHVPLHFCSRFGHHDIVKYLLQSDLEVQPHVVNIYGDTPLH LACYNGKFEVAKEIPISGTESLTKENJFSETAFHSACTYGKSIDLVKFLLDQNVININHQGRDG HTGLHSACYHGHERLVQFLLDNGADMNLVACDPSRSSGEKDEQTCLMWAYEKGHDAIVTLL KHYKRPQDELPCNEYSQPGGDGSYVSVPSPLGKIKSMTKEKADILLLRAGLPSHFHLQLSEIEF HEIIGSGSFGKVYKGRCRNKIVAIKRYRANTYCSKSDVDMFCREVSILCQLNHPCVIQFVGAC LNDPSQFAIVTQYISGGSLFSLLHEQKRILDLQSKLIIAVDVAKGMEYLHNLTQPIIHRDLNSHN ILLYEDGHAVVADFGESRFLQSLDEDNMTKQPGNLRWMAPEVFTQCTRYTIKADVFSYALCL WEILTGEIPFAHLKPAAAAADMAYBHERPPIGYSIPKPISSLLIRGWNACPE corresponding to amino acids 1-808 of TNI3K_HUMAN (SEQ. ID NO: 396), which also corresponds to amino acids 12-819 of C03950_{—}3_P12 (SEQ. ID NO:217), and a third amino acid sequence being at least 70%, optionally at least 80%, preferably at least 85%, more preferably at least 90% and most preferably at least 95% homologous to a polypeptide having the sequence AKSRPSHYPVSSVYTETLKKKNEDRFGMWEEYLRR (SEQ. ID NO: 356) corresponding to amino acids 820-854 of C03950_{—}3_P12 (SEQ. ID NO:217), wherein said first amino acid sequence, second amino acid sequence and third amino acid sequence are contiguous and in a sequential order.

B. An isolated polypeptide encoding for a head of C03950_{—}3_P12 (SEQ. ID NO:217), comprising a polypeptide being at least 70%, optionally at least about 80%, preferably at least about 85%, more preferably at least about 90% and most preferably at least about 95% homologous to the sequence AVRRGLREGGA (SEQ. ID NO: 342) of C03950_{—}3_P12 (SEQ. ID NO:217).

C. An isolated polypeptide encoding for an edge portion of C03950_{—}3_P12 (SEQ. ID NO:217), comprising an amino acid sequence being at least 70%, optionally at least about 80%, preferably at least about 85%, more preferably at least about 90% and most preferably at least about 95% homologous to the sequence AKSRPSHYPVSSVYTETLKKKNEDRFGMWIEYLRR (SEQ. ID NO: 356) of C03950_{—}3_P12 (SEQ. ID NO:217).

2. Comparison report between C03950_{—}3_P12 (SEQ. ID NO:217) and Q6MZS9_HUMAN (SEQ NO:211):

A. An isolated chimeric polypeptide encoding for C03950_{—}3_P12 (SEQ. ID NO:217), comprising a first amino acid sequence being at least 70%, optionally at least 80%, preferably at least 85%, more preferably at least 90% and most preferably at least 95%, homologous to a polypeptide having the sequence AVRRGLR (SEQ. ID NO: 344) corresponding to amino acids 1-7 of C03950_{—}3_P12 (SEQ. ID NO:217), a second amino acid sequence being at least 90% homologous to EGGAMAAARDPPEVSLREATQRKLRRFSELRGKLVARGEFWDIVAITAADEKQELAYNQQLS EKLKRKELPLGVQYHVFVDPAGAKIGNGGSTLCALQCLEKLYGDKWNSFULLIHSDEWKKK VSESYVIIIERLEDDLQIKEKELTELRNIEGSDEAFSKVNLNYRTENGLSLLHLCCICGGKKSHI RILMLKGLRPSRLTRNGFTALHLAVYKDNAELITSLLHSGADIQQVGYGGLTALHIATIAGHL EAADVLLQHGANVNIQDAVFFTPLIZAAYYGHEQVTRLLLKFGADVNVSGEVGDRPLHLAS AKGFLNIAKLLMEEGSKADVNAQDNEDHVPLHFCSRFGHHD corresponding to amino acids 14-367 of Q6MZS9_HUMAN (SEQ. ID NO:211), which also corresponds to amino acids 8-361 of C03950_{—}3_P12 (SEQ. ID NO:217), a bridging amino acid I corresponding to amino acid 362 of C03950_{—}31³12 (SEQ. ID NO:217), a third amino acid sequence being at least 90% homologous to VKYLLQSDLEVQPHVVNIYGDTPLHLACYNGKFEVAKEIIQISGTESLTKENIFSETAFHSACT YGKSIDLVKFLLDQNVININHQGRDGHTGLHSACYHGHIRLVQFLLDNGADM corresponding to amino acids 369-484 of Q6MZS9_HUMAN (SEQ. ID NO:211), which also corresponds to amino acids 363-478 of C03950_{—}3_P12 (SEQ. ID NO:217), a bridging amino acid N corresponding to amino acid 479 of C03950_{—}3_P12 (SEQ. ID NO:217), a fourth amino acid sequence being at least 90% homologous to LVACDPSRSSGEKDEQTCLMWAYEKGHDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNIIPCVIQFVGACLNDPSQFAIVTQYISGGSLFSLLHEQKRI LDLQSKLIIAVDVAKGMEYLHNLTQPIIHRDLN corresponding to amino acids 486-708 of Q6MZS9_HUMAN (SEQ. ID NO:211), which also corresponds to amino acids 480-702 of C03950_{—}3_P12 (SEQ. ID NO:217), and a fifth amino acid sequence being at least 70%, optionally at least 80%, preferably at least 85%, more preferably at least 90% and most preferably at least 95% homologous to a polypeptide having the sequence SHNILLYEDGHAVVADFGESRFLQSLDEDNMTKQPGNLRWMAPEVFTQCTRYTIKADVFSYA LCLWEMTGEIPFAHLKPAAAAADMAYHHIRPPIGYSIPKPISSLLIRGWNACPEAKSRPSHYPV SSVYTETLKKKNEDRFGMWIEYLRR (SEQ. ID NO: 358) corresponding to amino acids 703-854 of C03950_{—}3_P12 (SEQ. ID NO:217), wherein said first amino acid sequence, second amino acid sequence, bridging amino acid, third amino acid sequence, bridging amino acid, fourth amino acid sequence and fifth amino acid sequence are contiguous and in a sequential order.

B. An isolated polypeptide encoding for a head of C03950_{—}3_P12 (SEQ. ID NO:217), comprising a polypeptide being at least 70%, optionally at least about 80%, preferably at least about 85%, more preferably at least about 90% and most preferably at least about 95% homologous to the sequence AVRRGLR (SEQ. ID NO: 344) of C03950_{—}3_P12 (SEQ. ID NO:217).

C. An isolated polypeptide encoding for an edge portion of C03950_{—}3_P12 (SEQ. ID NO:217), comprising an amino acid sequence being at least 70%, optionally at least about 80%, preferably at least about 85%, more preferably at least about 90% and most preferably at least about 95% homologous to the sequence SHNELLYEDGHAVVADFGESRFLQSLDEDNMTKQPGNLRWMAPEVFTQCTRYTIKADVFSYA LCLWEILTGEWAHLKPAAAAADMAYHEIMPPIGYSIPKPISSLLIRGWNACPEAKSRPSHYPV SSVYTETLKKKNEDRFGMWIBYLRR (SEQ. ID NO: 358) of C03950_{—}3_P12 (SEQ. ID NO:217).

### 3. Comparison Report Between C03950_{—}3_P12 (SEQ. ID NO:217) and NI³_{—}057062 (SEQ. ID NO:210):

A. An isolated chimeric polypeptide encoding for C03950_{—}3_P12 (SEQ. ID NO:217), comprising a first amino acid sequence being at least 70%, optionally at least 80%, preferably at least 85%, more preferably at least 90% and most preferably at least 95%, homologous to a polypeptide having the sequence AVRRGLREGGAMAAARDPPEVSLREATQRKLRRFSELRGKLVARGEFWDIVAITAADEKQEL AYNQQLSEKLKRKELPLGVQYHVFVDPAGAKIGNGGSTLCALQCLEKLYGDKWNSFTILLIH S (SEQ. ID NO: 343) corresponding to amino acids 1-125 of C03950_{—}3_P12 (SEQ. ID NO:217), second amino acid sequence being at least 90% homologous to DEWKKKVSESYVITIERLEDDLQIKEKELTELRNIFGSDEAFSKVNLNYRTENGLSLLHLCCIC GGKKSHIRTLIALKGLRPSRLTRNGFTALHLAVYKDNAELITSLLHSGADIQQVGYGGLTALHE ATIAGHLEAADVLLQHGANVNIQDAVFFTPLHIAAYYGHEQVIRLLLKFGADVNVSGEVGDR PLHLASAKGFLNIAKLLMEEGSKADVNAQDNEDHVPLHFCSRFGHHDIVKYLLQSDLEVQPH VVNIYGDTPLHLACYNGKFEVAKEIPISGTESLTKENIFSETAFHSACTYGKSJDLVKFLLDQN VININHQGRDGHTGLHSACYHGHIRLVQFLLDNGADMNLVACDPSRSSGEKDEQTCLMWAY EKGHDAIVTLLKHYKRPQDELPCNEYSQPGGDGSYVSVPSPLGKIKSMTKEKADILLLRAGLP SHFHLQLSELEFHEIIGSGSFGKVYKGRCRNKIVATKRYRANTYCSKSDVDMFCREVSILCQLN HPCVIQFVGACLNDPSQFAIVTQYISGGSLFSLLHEQKRILDLQSKLIIAVDVAKGMEYLHNLT QPIRIRDLNSFINILLYEDGHAVVADFGESRFLQSLDEDNMTKQPGNLRWMAPEVFTQCTRYTI KADVFSYALCLWEILTGEIPFAHLKPAAAAADMAYHHIRPPIGYSIPKPISSLLIRGWNACPE corresponding to amino acids 14-707 of NP_{—}057062 (SEQ. ID NO:210), which also corresponds to amino acids 126-819 of C03950_{—}3_P12 (SEQ. ID NO:217), and a third amino acid sequence being at least 70%, optionally at least 80%, preferably at least 85%, more preferably at least 90% and most preferably at least 95% homologous to a polypeptide having the sequence AKSRPSHYPVSSVYTETLKKKNEDRFGMWMYLRR (SEQ. ID NO: 356) corresponding to amino acids 820-854 of C03950_{—}3_P12 (SEQ. ID NO:217), wherein said first amino acid sequence, second amino acid sequence and third amino acid sequence are contiguous and in a sequential order.

B. An isolated polypeptide encoding for a head of C03950_{—}3_P12 (SEQ. ID NO:217), comprising a polypeptide being at least 70%, optionally at least about 80%, preferably at least about 85%, more preferably at least about 90% and most preferably at least about 95% homologous to the sequence AVRRGLREGGAMAAARDPPEVSLREATQRKLRRFSELRGKLVARGEFWDIVAITAADEKQEL AYNQQLSEKLKRKELPLGVQYHVFVDPAGAKIGNGGSTLCALQCLEKLYGDKWNSFTILLIH S (SEQ. ID NO: 343) of C03950_{—}3_P12 (SEQ. ID NO:217).

The localization of the variant protein was determined according to results from a number of different software programs and analyses, including analyses from SignalP and other specialized programs. The variant protein is believed to be located intracellularly.

Variant protein C03950_{—}3_P12 (SEQ. ID NO:217) also has the following non-silent SNPs (Single Nucleotide Polymorphisms) as listed in Table 96, (given according to their position(s) on the amino acid sequence, with the alternative amino acid(s) listed.

TABLE 96
Amino acid mutations

| SNP position(s) on amino acid | |
|---|---|
| sequence | Alternative amino acid(s) |
| 119 | T -> P |

The variant protein has the following domains, as determined by using InterPro. The domains are described in Table 97:

TABLE 97
InterPro domain(s)

| Domain | | |
|---|---|---|
| description | Analysis type | Position(s) on protein |
| Protein kinase | BlastProDom | 575-777 |
| Ankyrin | FPrintScan | 279-291, 464-476 |
| Ankyrin | HMMPfam | 178-211, 212-244, 245-277, 278-310, |
| | | 311-345, 346-376, 381-414, 416-450, |
| | | 451-483, 493-525 |
| Protein kinase | HMMPfam | 575-832 |
| Tyrosine protein | HMMSmart | 575-833 |
| kinase | | |
| Serine | HMMSmart | 575-834 |
| Ankyrin | HMMSmart | 178-208, 212-241, 245-274, 278-307, |
| | | 311-342, 346-377, 381-412, 416-447, |
| | | 451-480, 493-522 |
| Protein kinase | ProfileScan | 575-837 |
| Ankyrin | ProfileScan | 212-244, 245-277, 281-310, 311-343, |
| | | 381-413, 451-483 |
| Ankyrin | ProfileScan | 170-513 |
| Protein kinase | ScanRegExp | 581-602 |

Variant protein C03950_{—}3_P12 (SEQ. ID NO:217) is encoded by the following transcript(s): C03950_{—}3_T10 (SEQ. ID NO:161), for which the coding portion starts at position 3 and ends at position 2564. The transcript also has the following SNPs as listed in Table 98 (given according to their position on the nucleotide sequence, with the alternative nucleic acid listed.

TABLE 98
Nucleic acid SNPs

| SNP position(s) on nucleotide | |
|---|---|
| sequence | Alternative nucleic acid(s) |
| 263 | A -> G |
| 357 | A -> C |
| 2677 | T -> C |

Variant protein C03950_{—}3_P13 (SEQ. ID NO:218) according to the present invention is encoded by transcript C03950_{—}3_T11 (SEQ. ID NO:162). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

1. Comparison report between C03950_{—}3_P13 (SEQ. ID NO:218) and TNI3K_HUMAN (SEQ. ID NO: 396):

A. An isolated chimeric polypeptide encoding for C03950_{—}3_P13 (SEQ. ID NO:218), comprising a first amino acid sequence being at least 70%, optionally at least 80%, preferably at least 85%, more preferably at least 90% and most preferably at least 95%, homologous to a polypeptide having the sequence MGNYKSRPTQTCT (SEQ. ID NO: 346) corresponding to amino acids 1-13 of C03950_{—}31³13 (SEQ. ID NO:218), a second amino acid sequence being at least 90% homologous to DEWKKKVSESYVITIERLEDDLQIKEKELTELRNIFGSDEAFSKVNLNYRTENGLSLLHLCCIC GGKKSHIRTLMLKGLRPSRLTRNGFTALHLAVYKDNAELITSLLHSGADIQQVGYGGLTALHI ATIAGHLEAADVLLQHGANVNIQDAVEFTPLIBAAYYGHEQVTRLLLKFGADVNVSGEVGDR PLHLASAKGELNIAKLLMEEGSKADVNAQDNEDHVPLHFCSRFGHBDIVKYLLQSDLEVQPH VVNTYGDTPUILACYNGKFEVAKEIIQISGTESLIXENIFSETAFHSACTYGKSMILVKFLLDQN VININHQGRDGHTGLHSACYHGHERLVQFLLDNGADMINTLVACDPSRSSGEKDEQTCLMWAY EKGHDAIVTLLKHYKRPQDELPCNEYSQPGGDGSYVSVPSPLGKIKSMTKEKADILLLRAGLP SHFHLQLSELEFHEIIGSGSFGKVYKGRCRNKIVAIKRYRANTYCSKSDVDMFCREVSILCQLN HPCVIQFVGACLNDPSQFAIVTQYISGGSLFSLLHEQKRILDLQSKLIIAVDVAKGMEYLHNLT QPBHRDLNSHNILLYEDGHAVVADFGESRFLQSLDEDNMTKQPGNLRWMAPEVETQCTRYTI KADVFSYALCLWEILTGEIPFAHLKPAAAAADMAYHHIRPPIGYSIPKPISSLLIRGWNACPE corresponding to amino acids 115-808 of TNI3K_HUMAN (SEQ. ID NO: 396), which also corresponds to amino acids 14-707 of C03950_{—}3_P13 (SEQ. ID NO:218), and a third amino acid sequence being at least 70%, optionally at least 80%, preferably at least 85%, more preferably at least 90% and most preferably at least 95% homologous to a polypeptide having the sequence AKSRPSHYPVSSVYTETLKKKNEDRFGMWIEYLRR (SEQ. ID NO: 356) corresponding to amino acids 708-742 of C03950_{—}3_P13 (SEQ. ID NO:218), wherein said first amino acid sequence, second amino acid sequence and third amino acid sequence are contiguous and in a sequential order.

B. An isolated polypeptide encoding for a head of C03950_{—}3_P13 (SEQ. ID NO:218), comprising a polypeptide being at least 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P13 (SEQ. ID NO:218).

C. An isolated polypeptide encoding for an edge portion of C03950_{—}3_P13 (SEQ. ID NO:218), comprising an amino acid sequence being at least 70%, optionally at least about 80%, preferably at least about 85%, more preferably at least about 90% and most preferably at least about 95% homologous to the sequence AKSRPSHYPVSSVYTETLKKKNEDRFGMIVIEYLRR (SEQ. ID NO: 356) of C03950_{—}3_P13 (SEQ. ID NO:218).

### 2. Comparison Report Between C03950_{—}3_P13 (SEQ. ID NO:218) and NP_{—}057062 (SEQ. ID NO:210):

A. An isolated chimeric polypeptide encoding for C03950_{—}3_P13 (SEQ. ID NO:218), comprising a first amino acid sequence being at least 90% homologous to MGNYKSRPTQTCTDEWKKKVSESYVITIERLEDDLQIKEKELTELRNIFGSDEAFSKVNLNYR TENGLSLLHLCCICGGKKSHIRTLMLKGLRPSRLTRNGFTALHLAVYKDNAELITSLLHSGADI QQVGYGGLTALHIATIAGHLEAADVLLQHGANVNIQDAVFFTPLHIAAYYGHEQVTRLLLKF GADVNVSGEVGDRPLHLASAKGFLNIAKLLMEEGSKADVNAQDNEDHVPLHFCSRFGHHDI VKYLLQSDLEVQPHVVNIYGDTPLIILACYNGKFEVAKELIQISGTESLTKENIFSETAFHSACT YGKSEDLVKFLLDQNVININHQGRDGHTGLHSACYHGHIRLVQFLLDNGADMNLVACDPSRS SGEKDEQTCLMWAYEKGIIDAIVTLLKHYKRPQDELPCNEYSQPGGDGSYVSVPSPLGKIKSM TKEKADILLLRAGLPSHFHLQLSEIEFHEIIGSGSFGKVYKGRCRNKIVAIKRYRANTYCSKSDV DMFCREVSILCQLNHPCVIQFVGACLNDPSQFAIVTQYISGGSLFSLLHEQKRILDLQSKLIIAV DVAKGMEYLHNLTQPIIHRDLNSHNILLYEDGHAVVADFGESRFLQSLDEDNMTKQPGIILR WMAPEVFTQCTRYTIKADVFSYALCLWEILTGEIPFAHLKPAAAAADMAYBEIRPPIGYSTKP ISSLLIRGWNACPE corresponding to amino acids 1-707 of NP_{—}057062 (SEQ. ID NO:210), which also corresponds to amino acids 1-707 of C03950_{—}3_P13 (SEQ. ID NO:218), and a second amino acid sequence being at least 70%, optionally at least 80%, preferably at least 85%, more preferably at least 90% and most preferably at least 95% homologous to a polypeptide having the sequence AKSRPSHYPVSSVYTETLKKKNEDRFGMWEBYLRR (SEQ. ID NO: 356) corresponding to amino acids 708-742 of C03950_{—}3_P13 (SEQ. ID NO:218), wherein said first amino acid sequence and second amino acid sequence are contiguous and in a sequential order.

### 4. Comparison Report Between C03950_{—}3_P13 (SEQ. ID NO:218) and Q6MZS9_HUMAN (SEQ. ID NO:211):

A. An isolated chimeric polypeptide encoding for C03950_{—}3_P13 (SEQ. ID NO:218), comprising a first amino acid sequence being at least 70%, optionally at least 80%, preferably at least 85%, more preferably at least 90% and most preferably at least 95%, homologous to a polypeptide having the sequence MGNYKSRPTQTCT (SEQ. ID NO: 346) corresponding to amino acids 1-13 of C03950_{—}3_P13 (SEQ. ID NO:218), a second amino acid sequence being at least 90% homologous to DEWKKKVSESYVITIERLEDDLQIKEKELTELRNEFGSDEAFSKVNLNYRTENGLSLLHLCCIC GGKKSHERTLMLKGLRPSRLTRNGFTALHLAVYKDNAELITSLLHSGADIQQVGYGGLTALHI ATIAGHLEAADVLLQHGANVNIQDAVFFTPLHEAAYYGHEQVTRLLLKFGADVNVSGEVGDR PLBLASAKGFLNIAKLLMEEGSKADVNAQDNEDHVPLITFCSRFGHHD corresponding to amino acids 132-367 of Q6MZS9_HUMAN (SEQ. ID NO:211), which also corresponds to amino acids 14-249 of C03950_{—}3_P13 (SEQ. ID NO:218), a bridging amino acid I corresponding to amino acid 250 of C03950_{—}3_P13 (SEQ. ID NO:218), a third amino acid sequence being at least 90% homologous to VKYLLQSDLEVQPHVVNIYGDTPLHLACYNGKFEVAKEIIQISGTESLTKENIFSETAFHSACT YGKSIDLVKFLLDQNVININHQGRDGHTGLHSACYHGHERLVQFLLDNGADM corresponding to amino acids 369-484 of Q6MZS9_HUMAN (SEQ. ID NO:211), which also corresponds to amino acids 251-366 of C03950_{—}3_P13 (SEQ. ID NO:218), a bridging amino acid N corresponding to amino acid 367 of C03950_{—}3_P13 (SEQ. ID NO:218), a fourth amino acid sequence being at least 90% homologous to LVACDPSRSSGEKDEQTCLMWAYEKGHDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNHPCVIQFVGACLNDPSQFAIVTQYISGGSLF SLLHEQKRI LDLQSKIIIAVDVAKGMEYLHNLTQPIIHRDLN corresponding to amino acids 486-708 of Q6MZS9_HUMAN (SEQ. ID NO:211), which also corresponds to amino acids 368-590 of C03950_{—}3_P13 (SEQ. ID NO:218), and a fifth amino acid sequence being at least 70%, optionally at least 80%, preferably at least 85%, more preferably at least 90% and most preferably at least 95% homol4ous to a polypeptide having the sequence SHNILLYEDGHAVVADFGESRFLQSLDEDNMTKQPGNLRWMAPEVFTQCTRYTIKADVFSYA LCLWEILTGEIPFAHLKPAAAAADMAYHHIRPPIGYSIPKPISSLLIRGWNACPEAKSRPSHYPV SSVYTETLKKKNEDRFGMWIEYLRR (SEQ. ID NO: 358) corresponding to amino acids 591-742 of C03950_{—}3_P13 (SEQ. ID NO:218), wherein said first amino acid sequence, second amino acid sequence, bridging amino acid, third amino acid sequence, bridging amino acid, fourth amino acid sequence and fifth amino acid seqUence are contiguous and in a sequential order.

B. An isolated polypeptide encoding for an edge portion of C03950_{—}3_P13 (SEQ. ID NO:218), comprising an amino acid sequence being at least 70%, optionally at least about 80%, preferably at least about 85%, more preferably at least about 90% and most preferably at least about 95% homologous to the sequence SHNILLYEDGHAVVADFGESRFLQSLDEDNMTKQPGNLRWMAPEVFTQCTRYTIKADVFSYA LCLWEILTGEIPFAHLKPAAAAADMAYHHIRPPIGYSIPKPISSLLIRGWNACPEAKSRPSHYPV SSVYTETLKKKNEDRFGMWIEYLRR (SEQ. ID NO: 358) of C03950_{—}3_P13 (SEQ. ID NO:218).

The localization of the variant protein was determined according to results from a number of different software programs and analyses, including analyses from SignalP and other specialized programs. The variant protein is believed to be located intracellularly.

The variant protein has the following domains, as determined by using InterPro. The domains are described in Table 99:

TABLE 99
InterPro domain(s)

| Domain | | |
|---|---|---|
| description | Analysis type | Position(s) on protein |
| Protein kinase | BlastProDom | 463-665 |
| Ankyrin | FPrintScan | 167-179, 352-364 |
| Ankyrin | HMMPfam | 66-99, 100-132, 133-165, 166-198, |
| | | 199-233, 234-264, 269-302, 304-338, |
| | | 339-371, 381-413 |
| Protein kinase | HMMPfam | 463-720 |
| Tyrosine protein | HMMSmart | 463-721 |
| kinase | | |
| Serine | HMMSmart | 463-722 |
| Ankyrin | HMMSmart | 66-96, 100-129, 133-162, 166-195, |
| | | 199-230, 234-265, 269-300, 304-335, |
| | | 339-368, 381-410 |
| Protein kinase | ProfileScan | 463-725 |
| Ankyrin | ProfileScan | 100-132, 133-165, 169-198, 199-231, |
| | | 269-301, 339-371 |
| Ankyrin | ProfileScan | 58-401 |
| Protein kinase | ScanRegExp | 469-490 |

Variant protein C03950_{—}3_P13 (SEQ. ID NO:218) is encoded by the following transcript(s): C03950_{—}3_T11 (SEQ. ID NO:162), for which the coding portion starts at position 389 and ends at position 2614. The transcript also has the following SNPs as listed in Table 100 (given according to their position on the nucleotide sequence, with the alternative nucleic acid listed.

TABLE 100
Nucleic acid SNPs

| SNP position(s) on nucleotide | |
|---|---|
| sequence | Alternative nucleic acid(s) |
| 2727 | T -> C |

Variant protein C03950_{—}3_P15 (SEQ. ID NO:219) according to the present invention is encoded by transcript C03950_{—}3_T13 (SEQ. ID NO:163). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

### 1. Comparison Report Between C03950_{—}3_P15 (SEQ. ID NO:219) and TNI3K_HUMAN (SEQ. ID NO: 396):

A. An isolated chimeric polypeptide encoding for C03950_{—}3_P15 (SEQ. ID NO:219), comprising a first amino acid sequence being at least 70%, optionally at least 80%, preferably at least 85%, more preferably at least 90% and most preferably at least 95%, homologous to a polypeptide having the sequence AVRRGLREGGA (SEQ. ID NO: 342) corresponding to amino acids 1-11 of C03950_{—}3_P15 (SEQ. ID NO:219), a second amino acid sequence being at least 90% homologous to MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHSDEWKKKVSES YVITIERLEDDLQIKEKELTELRNEFGSDEAFSKVNLNYRTENGLSLUILCCICGGKKSHIRTLM LKGLRPSRLTRNGFTALHLAVYKDNAELITSLLHSGADIQQVGYGGLTALMATIAGHLEAAD VLLQHGANVNIQDAVFFTPLIZAAYYGHEQVTRLLLKFGADVNVSGEVGDRPLHLASAKGFL NIAKLLMEEGSKADVNAQDNEDHVPLHFCSRFGHHDIVKYLLQSDLEVQPHVVNIYGDTPLH LACYNGKFEVAKEIIQISGTESLTKENIFSETAIESACTYGKSIDLVKFLLDQNVININHQGRDG HTGLHSACYHGBIRLVQFLLDNGADMNLVACDPSRSSGEKDEQTCLMWAYEKGHDAIVTLL KHYKRPQDELPCNEYSQPGGDGSYVSVPSPLGKIKSMTKEKADILLLRAGLPSHFHLQLSEIEF HEIIGSGSFGKVYKGRCRNKIVAIKRYRANTYCSKSDVDMFCREVSILCQLNHPCVIQFVGAC LNDPSQFAIVTQYISGGSLFSLLHEQKRILDLQSKLIIAVDVAKGMEYLHNLTQPIIHRDLN corresponding to amino acids 1-691 of TNI3K_HUMAN (SEQ. ID NO: 396), which also corresponds to amino acids 12-702 of C03950_{—}3_P15 (SEQ. ID NO:219), and a third amino acid sequence being at least 70%, optionally at least 80%, preferably at least 85%, more preferably at least 90% and most preferably at least 95% homologous to a polypeptide having the sequence RYFFPK (SEQ. ID NO: 364) corresponding to amino acids 703-708 of C03950_{—}3_P15 (SEQ. ID NO:219), wherein said first amino acid sequence, second amino acid sequence and third amino acid sequence are contiguous and in a sequential order.

B. An isolated polypeptide encoding for a head of C03950_{—}3_P15 (SEQ. ID NO:219), comprising a polypeptide being at least 70%, optionally at least about 80%, preferably at least about 85%, more preferably at least about 90% and most preferably at least about 95% homologous to the sequence AVRRGLREGGA (SEQ. ID NO: 342) of C03950_{—}3_P15 (SEQ. ID NO:219).

C. An isolated polypeptide encoding for an edge portion of C03950_{—}3_P15 (SEQ. ID NO:219), comprising an amino acid sequence being at least 70%, optionally at least about 80%, preferably at least about 85%, more preferably at least about 90% and most preferably at least about 95% homologous to the sequence RYFFPK (SEQ. ID NO: 364) of C03950_{—}3_P15 (SEQ. ID NO:219).

2. Comparison report between C03950_{—}3_P15 (SEQ. ID NO:219) and Q6MZS9_HUMAN (SEQ. ID NO:211):

A. An isolated chimeric polypeptide encoding for C03950_{—}3_P15 (SEQ. ID NO:219), comprising a first amino acid sequence being at least 70%, optionally at least 80%, preferably at least 85%, more preferably at least 90% and most preferably at least 95%, homologous to a polypeptide having the sequence AVRRGLR (SEQ. ID NO: 344) corresponding to amino acids 1-7 of C03950_{—}3_P15 (SEQ. ID NO:219), a second amino acid sequence being at least 90% homologous to EGGAMAAARDPPEVSLREATQRKLRRFSELRGKLVARGEFWDIVAITAADEKQELAYNQQLS EKLKRKELPLGVQYHVFVDPAGAKIGNGGSTLCALQCLEKLYGDKWNSFTILLIHSDEWKKK VSESYVITTERLEDDLQIKEKELTELRNIFGSDEAFSKVNLNYRTENGLSLLHLCCICGGKKSHI RTLMLKGLRPSRLTRNGFTALBLAVYKDNAELITSLLHSGADIQQVGYGGLTALHIATIAGHL EAADVLLQHGANVNIQDAVFFTPLHIAAYYGHEQVTRLLLKFGADVNVSGEVGDRPLIALAS AKGFLNIAKLLMEEGSKADVNAQDNEDHVPLHFCSRFGHHD corresponding to amino acids 14-367 of Q6MZS9_HUMAN (SEQ. ID NO:211), which also corresponds to amino acids 8-361 of C03950_{—}3_P15 (SEQ. ID NO:219), a bridging amino acid I corresponding to amino acid 362 of C03950_{—}3_P15 (SEQ. ID NO:219), a third amino acid sequence being at least 90% homologous to VKYLLQSDLEVQPHVVNIYGDTPLHLACYNGKFEVAKEIIQISGTESLTKENIFSETAFHSACT YGKSIDLVKFLLDQNVININHQGRDGHTGLHSACYHGHERLVQFLLDNGADM corresponding to amino acids 369-484 of Q6MZS9_HUMAN (SEQ. ID NO:211), which also corresponds to amino acids 363-478 of C03950_{—}3_P15 (SEQ. ID NO:219), a bridging amino acid N corresponding to amino acid 479 of C03950_{—}3_P15 (SEQ. ID NO:219), and a fourth amino acid sequence being at least 90% homologous to LVACDPSRSSGEKDEQTCLMWAYEKGHDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNHPCVIQFVGACLNDPSQFAIVTQYISGGSLFSLLHEQKRI LDLQSKLLIAVDVAKGKEYLIINLTQPIIHRDLNRYFFPK corresponding to amino acids 486-714 of Q6MZS9_HUMAN (SEQ. ID NO:211), which also corresponds to amino acids 480-708 of C03950_{—}3_P15 (SEQ. ID NO:219), wherein said first amino acid sequence, second amino acid sequence, bridging amino acid, third amino acid sequence, bridging amino acid and fourth amino acid sequence are contiguous and in a sequential order.

B. An isolated polypeptide encoding for a head of C03950_{—}3_P15 (SEQ. ID NO:219), comprising a polypeptide being at least 70%, optionally at least about 80%, preferably at least about 85%, more preferably at least about 90% and most preferably at least about 95% homologous to the sequence AVRRGLR (SEQ. ID NO: 344) of C03950_{—}3_P15 (SEQ. ID NO:219).

### 3. Comparison Report Between C03950_{—}3_P15 (SEQ. ID NO:219) and NP_{—}057062 (SEQ. ID NO:210):

A. An isolated chimeric polypeptide encoding for C03950_{—}3_P15 (SEQ. ID NO:219), comprising a first amino acid sequence being at least 70%, optionally at least 80%, preferably at least 85%, more preferably at least 90% and most preferably at least 95%, homologous to a polypeptide having the sequence AVRRGLREGGAMAAARDPPEVSLREATQRKLRRFSELRGKLVARGEFWDIVAITAADEKQEL AYNQQLSEKLKRKELPLGVQYHVFVDPAGAKIGNGGSTLCALQCLEKLYGDKWNSFTILLTH S (SEQ. ID NO: 343) corresponding to amino acids 1-125 of C03950_{—}3_P15 (SEQ. LD NO:219), second amino acid sequence being at least 90% homologous to DEWKKKVSESYVITIERLEDDLQIKEKELTELRNTFGSDEAFSKVNLNYRTENGLSLLHLCCIC GGKKSHIRTLMLKGLRPSRLTRNGFTALHLAVYKDNAELITSLLHSGADIQQVGYGGLTALHI ATIAGHLEAADVLLQHGANVNIQDAVFFTPLHIAAYYGHEQVTRLLLKFGADVNVSGEVGDR PLHLASAKGFLNIAKLLMEEGSKADVNAQDNEDHVPLHFCSRFGHHDIVKYLLQSDLEVQPH VVNIYGDTPLHLACYNGKFEVAKEIIQISGTESLTKENIFSETAFHSACTYGKSIDLVKFILDQN VININHQGRDGHTGLHSACYHGHIRLVQFLLDNGADMNLVACDPSRSSGEKDEQTCLMWAY EKGHDAIVTLLKHYKRPQDELPCNEYSQPGGDGSYVSVPSPLGKIKSMTKEKADILLLRAGLP SHFHLQLSEIEFHEIIGSGSFGKVYKGRCRNKIVALKRYRANTYCSKSDVDMFCREVSILCQLN HPCVIQFVGACLNDPSQFAIVTQYISGGSLFSLLHEQKRILDLQSKLHAVDVAKGMEYLHNLT QPIIHRDLN corresponding to amino acids 14-590 of NP_{—}057062 (SEQ. ID NO:210), which also corresponds to amino acids 126-702 of C03950_{—}3 1315 (SEQ. ID NO:219), and a third amino acid sequence being at least 70%, optionally at least 80%, preferably at least 85%, more preferably at least 90% and most preferably at least 95% homologous to a polypeptide having the sequence RYFFPK (SEQ. ID NO: 364) corresponding to amino acids 703-708 of C03950_{—}3_P15 (SEQ. ID NO:219), wherein said first amino acid sequence, second amino acid sequence and third amino acid sequence are contiguous and in a sequential order.

B. An isolated polypeptide encoding for a head of C03950_{—}3_P15 (SEQ. ID NO:219), comprising a polypeptide being at least 70%, optionally at least about 80%, preferably at least about 85%, more preferably at least about 90% and most preferably at least about 95% homologous to the sequence AVRRGLREGGAMAAARDPPEVSLREATQRKLRRFSELRGKLVARGEFWDIVAITAADEKQEL AYNQQLSEKLKRKELPLGVQYHVFVDPAGAKIGNGGSTLCALQCLEKLYGDKWNSFTILLIH S (SEQ. ID NO: 343) of C03950_{—}3_P15 (SEQ. ID NO:219).

The localization of the variant protein was determined according to results from a number of different software programs and analyses, including analyses from SignalP and other specialized programs. The variant protein is believed to be located intracellularly.

Variant protein C03950_{—}3_P15 (SEQ. ID NO:219) also has the following non-silent SNPs (Single Nucleotide Polymorphisms) as listed in Table 101, (given according to their position(s) on the amino acid sequence, with the alternative amino acid(s) listed.

TABLE 101
Amino acid mutations

| SNP position(s) on amino acid | |
|---|---|
| sequence | Alternative amino acid(s) |
| 119 | T -> P |

The variant protein has the following domains, as determined by using InterPro. The domains are described in Table 102:

TABLE 102
InterPro domain(s)

| Domain | | |
|---|---|---|
| description | Analysis type | Position(s) on protein |
| Protein kinase | BlastProDom | 575-704 |
| Ankyrin | FPrintScan | 279-291, 464-476 |
| Ankyrin | HMMPfam | 178-211, 212-244, 245-277, 278-310, |
| | | 311-345, 346-376, 381-414, 416-450, |
| | | 451-483, 493-525 |
| Tyrosine protein | HMMSmart | 575-708 |
| kinase | | |
| Serine | HMMSmart | 575-705 |
| Ankyrin | HMMSmart | 178-208, 212-241, 245-274, 278-307, |
| | | 311-342, 346-377, 381-412, 416-447, |
| | | 451-480, 493-522 |
| Protein kinase | ProfileScan | 575-708 |
| Ankyrin | ProfileScan | 212-244, 245-277, 281-310, 311-343, |
| | | 381-413, 451-483 |
| Ankyrin | ProfileScan | 170-513 |
| Protein kinase | ScanRegExp | 581-602 |

Variant protein C03950_{—}3_P15 (SEQ. ID NO:219) is encoded by the following transcript(s): C03950_{—}3_T13 (SEQ. ID NO:163), for which the coding portion starts at position 3 and ends at position 2126. The transcript also has the following SNPs as listed in Table 103 (given according to their position on the nucleotide sequence, with the alternative nucleic acid listed.

TABLE 103
Nucleic acid SNPs

| SNP position(s) on nucleotide | |
|---|---|
| sequence | Alternative nucleic acid(s) |
| 263 | A -> G |
| 357 | A -> C |
| 7207 | C -> A |

Variant protein C03950_{—}3_P17 (SEQ. ID NO:220) according to the present invention is encoded by transcript C03950_{—}3_T15 (SEQ. ID NO:164). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

### 1. Comparison Report Between C03950_{—}3_P17 (SEQ. ID NO:220) and TNI3K_HUMAN (SEQ. ID NO: 396):

A. An isolated chimeric polypeptide encoding for C03950_{—}3_P17 (SEQ. ID NO:220), comprising a first amino acid sequence being at least 70%, optionally at least 80%, preferably at least 85%, more preferably at least 90% and most preferably at least 95%, homologous to a polypeptide having the sequence MGNYKSRPTQTCT (SEQ. ID NO: 346) corresponding to amino acids 1-13 of C03950_{—}3_P17 (SEQ. ID NO:220), a second amino acid sequence being at least 90% homologous to DEWKKKVSESYVITTERLEDDLQLKEKELTELRNIFGSDEAFSKVNLNYRTENGLSLLHLCCIC GGKKSHIRTLMELKGLRPSRLTRNGFTALBLAVYKDNAELITSLLHSGADIQQVGYGGLTALHI ATIAGHLEAADVLLQHGANVNIQDAVFFTPLHIAAYYGITEQVTRLLLKFGADVNVSGEVGDR PLHLASAKGFLNIAKLLMEEGSKADVNAQDNEDHVPLHFCSRFGHHDIVKYLLQSDLEVQPH VVNIYGDTPLHLACYNGKFEVAKEIIQISGTESLTKENIFSETAPHSACTYGKSIDLVKFLLDQN VININHQGRDGHTGLHSACYHGHIRLVQFLLDNGADMNLVACDPSRSSGEKDEQTCLMWAY EKGHDAIVTLLKHYKRPQDELPCNEYSQPGGDGSYVSVPSPLGKIKSMTKEKADILLLRAGLP SHFHLQLSEIEFHEIIGSGSFGKVYKGRCRNKIVAIKRYRANTYCSKSDVDMFCREVSILCQLN HPCVIQFVGACLNDPSQFAIVTQYISGGSLFSLLHEQKRILDLQSKLILAVDVAKGMEYLHNLT QPIIHRDLN corresponding to amino acids 115-691 of TNI3K_HUMAN (SEQ. ID NO: 396), which also corresponds to amino acids 14-590 of C03950_{—}3_P17 (SEQ. ID NO:220), and a third amino acid sequence being at least 70%, optionally at least 80%, preferably at least 85%, more preferably at least 90% and most preferably at least 95% homologous to a polypeptide having the sequence RCCTGWLSCYHPD (SEQ. ID NO: 368) corresponding to amino acids 591-603 of C03950_{—}3_P17 (SEQ. ID NO:220), wherein said first amino acid sequence, second amino acid sequence and third amino acid sequence are contiguous and in a sequential order.

B. An isolated polypeptide encoding for a head of C03950_{—}3_P17 (SEQ. ID NO:220), comprising a polypeptide being at least 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P17 (SEQ. ID NO:220).

C. An isolated polypeptide encoding for an edge portion of C03950_{—}3_P1 7 (SEQ. ID NO:220), comprising an amino acid sequence being at least 70%, optionally at least about 80%, preferably at least about 85%, more preferably at least about 90% and most preferably at least about 95% homologous to the sequence RCCTGWLSCYHPD (SEQ. ID NO: 368) of C03950_{—}3_P17 (SEQ. ID NO:220).

2. Comparison report between C03950_{—}3_P17 (SEQ. ID NO:220) and Q6MZS9_HUMAN (SEQ. ID NO:211):

A. An isolated chimeric polypeptide encoding for C03950_{—}3_P17 (SEQ. ID NO:220), comprising a first amino acid sequence being at least 70%, optionally at least 80%, preferably at least 85%, more preferably at least 90% and most preferably at least 95%, homologous to a polypeptide having the sequence MGNYKSRPTQTCT (SEQ. ID NO: 346) corresponding to amino acids 1-13 of C03950_{—}3_P17 (SEQ. ID NO:220), a second amino acid sequence being at least 90% homologous to DEWKKKVSESYVMERLEDDLQIKEKELTELRNIFGSDEAFSKVNLNYRTENGLSLLHLCCIC GGKKSHIRTLMLKGLRPSRLTRNGFTALHLAVYKDNAELITSLLHSGADIQQVGYGGLTALHE ATIAGHLEAADVLLQHGANVNIQDAVFFTPLHIAAYYGHEQVTRLLLKFGADVNVSGEVGDR PLHLASAKGFLNIAKLLMEEGSKADVNAQDNEDHVPLHFCSRFGHHD corresponding to amino acids 132-367 of Q6MZS9_HUMAN (SEQ. ID NO:211), which also corresponds to amino acids 14-249 of C03950_{—}3_P17 (SEQ. ID NO:220), a bridging amino acid I corresponding to amino acid 250 of C03950_{—}3_P17 (SEQ. ID NO:220), a third amino acid sequence being at least 90% homologous to VKYLLQSDLEVQPHVVNIYGDTPLHLACYNGKFEVAKEIIQISGTESLTKENIFSETAFHSACT YGKSIDLVKFLLDQNVININHQGRDGHTGLHSACYHGHIRLVQFLLDNGADM corresponding to amino acids 369-484 of Q6MZS9_HUMAN (SEQ. ID NO:211), which also corresponds to amino acids 251-366 of C03950_{—}3_P17 (SEQ. ID NO:220), a bridging amino acid N corresponding to amino acid 367 of C03950_{—}3_P17 (SEQ. ID NO:220), a fourth amino acid sequence being at least 90% homologous to LVACDPSRSSGEKDEQTCLMWAYEKGHDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNHPCVIQFVGACLNDPSQFAIVTQYISGGSLFSLLHEQKRI LDLQSKLIIAVDVAKGMEYLHNLTQPIIHRDLNR corresponding to amino acids 486-709 of Q6MZS9_HUMAN (SEQ. ID NO:211), which also corresponds to amino acids 368-591 of C03950_{—}3_P17 (SEQ. ID NO:220), and a fifth amino acid sequence being at least 70%, optionally at least 80%, preferably at least 85%, more preferably at least 90% and most preferably at least 95% homologous to a polypeptide having the sequence CCTGWLSCYHPD (SEQ. ID NO: 369) corresponding to amino acids 592-603 of C03950_{—}3_P17 (SEQ. ID NO:220), wherein said first amino acid sequence, second amino acid sequence, bridging amino acid, third amino acid sequence, bridging amino acid, fourth amino acid sequence and fifth amino acid sequence are contiguous and in a sequential order.

B. An isolated polypeptide encoding for a head of C03950_{—}3_P17 (SEQ. ID NO:220), comprising a polypeptide being at least 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P17 (SEQ. ID NO:220).

C. An isolated polypeptide encoding for an edge portion of C03950_{—}3_P17 (SEQ. ID NO:220), comprising an amino acid sequence being at least 70%, optionally at least about 80%, preferably at least about 85%, more preferably at least about 90% and most preferably at least about 95% homologous to the sequence CCTGWLSCYHPD (SEQ. ID NO: 369) of C03950_{—}3_P17 (SEQ. ID NO:220).

### 3. Comparison Report Between C03950_{—}3_P17 (SEQ. ID NO:220) and NP_{—}057062 (SEQ. ID NO:210):

A. An isolated chimeric polypeptide encoding for C03950_{—}3_P17 (SEQ. ID NO:220), comprising a first amino acid sequence being at least 90% homologous to MGNYKSRPTQTCTDEWKKKVSESYVITIERLEDDLQIKEKELTELRNIFGSDEAFSKVNLNYR TENGLSLLHLCCKGGKKSHIRTLMIKGLRPSRLTRNGFTALHLAVYKDNAELITSLLHSGADI QQVGYGGLTALMAITAGHLEAADVLLQHGANVNIQDAVFFTPLHIANYYGHEQVTRLLLKF GADVNVSGEVGDRPLHLASAKGFLNIAKLLMEEGSKADVNAQDNEDHVPLHFCSRFGHHDI VKYLLQSDLEVQPHVVNIYGDTPUILACYNGKFEVAKEIIQISGTESLTKENIFSETAFHSACT YGKSIDLVKFLLDQNVININHQGRDGHTGLHSACYHGHIRLVQFLLDNGADMNLVACDPSRS SGEKDEQTCLMWAYEKGHDAIVTLLKHYKRPQDELPCNEYSQPGGDGSYVSVPSPLGKIKSM TKEKADILLLRAGLPSIIFHLQLSEIEFHEIIGSGSFGKVYKGRCRNKIVAIKRYRANTYCSKSDV DMFCREVSILCQLNHPCVIQFVGACLNDPSQFAIVTQYISGGSLFSLLBEQKRILDLQSKLIIAV DVAKGMEYLHNLTQPIIHRDLN corresponding to amino acids 1-590 of NP_{—}057062 (SEQ. ID NO:210), which also corresponds to amino acids 1-590 of C03950_{—}3_P17 (SEQ. ID NO:220), a second amino acid sequence being at least 70%, optionally at least 80%, preferably at least 85%, more preferably at least 90% and most preferably at least 95% homologous to a polypeptide having the sequence RCCTGWLSCYHPD (SEQ. ID NO: 368) corresponding to amino acids 591-603 of C03950_{—}3_P17 (SEQ. ID NO:220), wherein said first amino acid sequence and second amino acid sequence are contiguous and in a sequential order.

The localization of the variant protein was determined according to results from a number of different software programs and analyses, including analyses from SignalP and other specialized programs. The variant protein is believed to be located intracellularly.

The variant protein has the following domains, as determined by using InterPro. The domains are described in Table 104:

TABLE 104
InterPro domain(s)

| Domain | | |
|---|---|---|
| description | Analysis type | Position(s) on protein |
| Protein kinase | BlastProDom | 463-590 |
| Ankyrin | FPrintScan | 167-179, 352-364 |
| Ankyrin | HMMPfam | 66-99, 100-132, 133-165, 166-198, |
| | | 199-233, 234-264, 269-302, 304-338, |
| | | 339-371, 381-413 |
| Tyrosine protein | HMMSmart | 463-602 |
| kinase | | |
| Serine | HMMSmart | 463-603 |
| Ankyrin | HMMSmart | 66-96, 100-129, 133-162, 166-195, |
| | | 199-230, 234-265, 269-300, 304-335, |
| | | 339-368, 381-410 |
| Protein kinase | ProfileScan | 463-603 |
| Ankyrin | ProfileScan | 100-132, 133-165, 169-198, 199-231, |
| | | 269-301, 339-371 |
| Ankyrin | ProfileScan | 58-401 |
| Protein kinase | ScanRegExp | 469-490 |

Variant protein C03950_{—}3_P17 (SEQ. ID NO:220) is encoded by the following transcript(s): C03950_{—}3_T15 (SEQ. ID NO:164), for which the coding portion starts at position 389 and ends at position 2197.

Variant protein C03950_{—}3_T³19 (SEQ. ID NO:221) according to the present invention is encoded by transcript C03950_{—}3_T17 (SEQ. ID NO:165). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

### 1. Comparison Report Between C03950_{—}3_P19 (SEQ. ID NO:221) and TNI3K HUMAN (SEQ. ID NO: 396):

A. An isolated chimeric polypeptide encoding for C03950_{—}3_P19 (SEQ. ID NO:221), comprising a first amino acid sequence being at least 70%, optionally at least 80%, preferably at least 85%, more preferably at least 90% and most preferably at least 95%, homologous to a polypeptide having the sequence MGNYKSRPTQTCT (SEQ. ID NO: 346) corresponding to amino acids 1-13 of C03950_{—}3_P19 (SEQ. ID NO:221), a second amino acid sequence being at least 90% homologous to DEWKKKVSESYVITIERLEDDLQIKEKELTELRNIFGSDEAFSKVNLNYRTENGLSLLHLCCIC GGKKSHIRTLNILKGLRPSRLTRNGFTALHLAVYKDNAELITSLLHSGADIQQVGYGGLTALHI ATIAGHLEAADVLLQHGANVNIQDAVFFTPLHIAAYYGHEQVTRLLLKFGADVNVSGEVGDR PLHLASAKGFLNIAKLLMEEGSKADVNAQDNEDHVPLHFCSRFGHHDIVKYLLQSDLEVQPH VVNIYGDTPLHLACYNGKFEVAKEIIQISGTESLTKENIFSETAPHSACTYGKSIDLVKFLLDQN VININHQGRDGHTGLHSACYHGHIRLVQFLLDNGADMNLVACDPSRSSGEKDEQTCLMWAY EKGHDAIVTLLKHYKRPQDELPCNEYSQPGGDGSYVSVPSPLGKIKSMTKEKADILLLRAGLP SHFHLQLSEIEFHEIIGSGSFGKVYKGRCRNKIVAIKRYRANTYCSKSDVDMFCREVSILCQLN HPCVIQFVGACLNDPSQFAIVTQYISGGSLFSLLHEQKRILDLQSKLITAVDVAKGMEYLHNLT QPIIHRDLN corresponding to amino acids 115-691 of TNI3K_HUMAN (SEQ. ID NO: 396), which also corresponds to amino acids 14-590 of C03950_{—}3_P19 (SEQ. ID NO:221), and a third amino acid sequence being at least 70%, optionally at least 80%, preferably at least 85%, more preferably at least 90% and most preferably at least 95% homologous to a polypeptide having the sequence RAS (SEQ. ID NO: 370) corresponding to amino acids 591-593 of C03950_{—}3_P19 (SEQ. ID NO:221), wherein said first amino acid sequence, second amino acid sequence and third amino acid sequence are contiguous and in a sequential order.

B. An isolated polypeptide encoding for an edge portion of C03950_{—}3_P19 (SEQ. ID NO:221), comprising an amino acid sequence being at least 70%, optionally at least about 80%, preferably at least about 85%, more preferably at least about 90% and most preferably at least about 95% homologous to the sequence RAS (SEQ. ID NO: 370) of C03950_{—}3_P19 (SEQ. ID NO:221).

### 2. Comparison Report Between C03950_{—}3_P19 (SEQ. ID NO:221) and Q6MZS9_HUMAN (SEQ. ID NO:211):

A. An isolated chimeric polypeptide encoding for C03950_{—}3_P19 (SEQ. ID NO:221), comprising a first amino acid sequence being at least 70%, optionally at least 80%, preferably at least 85%, more preferably at least 90% and most preferably at least 95%, homologous to a polypeptide having the sequence MGNYKSRPTQTCT (SEQ. ID NO: 346) corresponding to amino acids 1-13 of C03950_{—}3_P19 (SEQ. ID NO:221), a second amino acid sequence being at least 90% homologous to DEWKKKVSESYVITIERLEDDLQIKEKELTELRNIFGSDEAFSKVNLNYRTENGLSLLHLCCIC GGKKSILIRTLMLKGLRPSRLTRNGFTALHLAVYKDNAELITSLLHSGADIQQVGYGGLTALHE ATIAGHLEAADVLLQHGANVNIQDAVFFTPLHIAAYYGHEQVTRLLLKFGADVNVSGEVGDR PLHLASAKGFLNIAKLLMEEGSKADVNAQDNEDHVPLHFCSRFGHHD corresponding to amino acids 132-367 of Q6MZS9_HUMAN (SEQ. ID NO:211), which also corresponds to amino acids 14-249 of C03950_{—}3_P19 (SEQ. ID NO:221), a bridging amino acid I corresponding to amino acid 250 of C03950_{—}3_P19 (SEQ. ID NO:221), a third amino acid sequence being at least 90% homologous to VKYLLQSDLEVQPHVVNIYGDTPLHLACYNGKFEVAKEIIQISGTESLTKENIFSETAFHSACT YGKSIDINKFLLDQNVININHQGRDGHTGLHSACYHGHERLVQFLLDNGADM corresponding to amino acids 369-484 of Q6MZS9_HUMAN (SEQ. ID NO:211), which also corresponds to amino acids 251-366 of C03950_{—}3_P19 (SEQ. ID NO:221), a bridging amino acid N corresponding to amino acid 367 of C03950_{—}3_P19 (SEQ. ID NO:221), a fourth amino acid sequence being at least 90% homologous to LVACDPSRSSGEKDEQTCLMWAYEKGEDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNHPCVIQFVGACLNDPSQFAIVTQYISGGSLFSLLHEQKRI LDLQSKLIIAVDVAKGMEYLHNLTQPIIHRDLNR corresponding to amino acids 486-709 of Q6MZS9_HUMAN (SEQ. ID NO:211), which also corresponds to amino acids 368-591 of C03950_{—}3_P19 (SEQ. ID NO:221), and a fifth amino acid sequence being at least 70%, optionally at least 80%, preferably at least 85%, more preferably at least 90% and most preferably at least 95% homologous to a polypeptide having the sequence AS corresponding to amino acids 592-593 of C03950_{—}3_P19 (SEQ. ID NO:221), wherein said first amino acid sequence, second amino acid sequence, bridging amino acid, third amino acid sequence, bridging amino acid, fourth amino acid sequence and fifth amino acid sequence are contiguous and in a sequential order.

### 3. Comparison Report Between C03950_{—}3_P19 (SEQ. ID NO:221) and NP_{—}057062 (SEQ. ID NO:210):

A. An isolated chimeric polypeptide encoding for C03950_{—}3_P19 (SEQ. 10 NO:221), comprising a first amino acid sequence being at least 90% homologous to MGNYKSRPTQTCTDEWKKKVSESYVITIERLEDDLQIKEKELTELRNIFGSDEAFSKVNLNYR TENGLSLLHLCCKGGKKSHIRTLMLKGLRPSRLTRNGFTALHLAVYKDNAELITSLLHSGADI QQVGYGGLTALHIATIAGHLEAADVLLQHGANVNIQDAVFFTPLEHAAYYGHEQVTRLLLKF GADVNVSGEVGDRPLHLASAKGFLNIAKLLMEEGSKADVNAQDNEDHVPLHFCSRFGHHDI VKYLLQSDLEVQPHVVNIYGDTPLHLACYNGKFEVAKEIIQISGTESLTKENIFSETAFHSACT YGKSIDLVKFLLDQNVINTNHQGRDGHTGLHSACYHGHERLVQFLLDNGADMNLVACDPSRS SGEKDEQTCLMWAYEKGHDAIVTLLKHYKRPQDELPCNEYSQPGGDGSYVSVPSPLGKIKSM TKEKADILLLRAGLPSHFHLQLSEIEFHEIIGSGSFGKVYKGRCRNKIVAIKRYRANTYCSKSDV DMFCREVSILCQLNHPCVIQFVGACLNDPSQFAIVTQYISGGSLFSLLHEQKRILDLQSKLHAV DVAKGMEYLHNLTQPIIHRDLN corresponding to amino acids 1-590 of NP_{—}057062 (SEQ. ID NO:210), which also corresponds to amino acids 1-590 of C03950_{—}3_P19 (SEQ. ID NO:221), and a second amino acid sequence being at least 70%, optionally at least 80%, preferably at least 85%, more preferably at least 90% and most preferably at least 95% homologous to a polypeptide having the sequence RAS (SEQ. ID NO: 370) corresponding to amino acids 591-593 of C03950_{—}3_P19 (SEQ. ID NO:221), wherein said first amino acid sequence and second amino acid sequence are contiguous and in a sequential order.

The localization of the variant protein was determined according to results from a number of different software programs and analyses, including analyses from SignalP and other specialized programs. The variant protein is believed to be located intracellularly.

The variant protein has the following domains, as determined by using InterPro. The domains are described in Table 105:

TABLE 105
InterPro domain(s)

| Domain | | |
|---|---|---|
| description | Analysis type | Position(s) on protein |
| Protein kinase | BlastProDom | 463-590 |
| Ankyrin | FPrintScan | 167-179, 352-364 |
| Ankyrin | HMMPfam | 66-99, 100-132, 133-165, 166-198, |
| | | 199-233, 234-264, 269-302, 304-338, |
| | | 339-371, 381-413 |
| Tyrosine protein | HMMSmart | 463-593 |
| kinase | | |
| Serine | HMMSmart | 463-592 |
| Ankyrin | HMMSmart | 66-96, 100-129, 133-162, 166-195, |
| | | 199-230, 234-265, 269-300, 304-335, |
| | | 339-368, 381-410 |
| Protein kinase | ProfileScan | 463-593 |
| Ankyrin | ProfileScan | 100-132, 133-165, 169-198, 199-231, |
| | | 269-301, 339-371 |
| Ankyrin | ProfileScan | 58-401 |
| Protein kinase | ScanRegExp | 469-490 |

Variant protein C03950_{—}3_P19 (SEQ. ID NO:221) is encoded by the following transcript(s): C03950_{—}3_T17 (SEQ. ID NO:165), for which the coding portion starts at position 389 and ends at position 2167.

Variant protein C03950_{—}3_P20 (SEQ. ID NO:222) according to the present invention is encoded by transcript C03950_{—}3_T18 (SEQ. ID NO:166). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

### 1. Comparison Report Between C03950_{—}3_P20 (SEQ. ID NO:222) and TNI3K_HUMAN (SEQ. ID NO: 396):

A. An isolated chimeric polypeptide encoding for C03950_{—}3_P20 (SEQ. ID NO:222), comprising a first amino acid sequence being at least 70%, optionally at least 80%, preferably at least 85%, more preferably at least 90% and most preferably at least 95%, homologous to a polypeptide having the sequence AVRRGLREGGA (SEQ. ID NO: 342) corresponding to amino acids 1-11 of C03950_{—}3_P20 (SEQ. ID NO:222), a second amino acid sequence being at least 90% homologous to MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHSDEWKKKVSES YVITIERLEDDLQIKEKELTELRNIFGSDEAFSKVNLNYRTENGLSLLHLCCKGGKKSHIRTLM LKGLRPSRLTRNGFTALHLAVYKDNAELITSLLHSGADIQQVGYGGLTALHIATIAGHLEAAD VLLQHGANVNIQDAVFFTPLIIIAAYYGHEQVTRLLLKFGADVNVSGEVGDRPLHLASAKGFL NIAKLLMEEGSKADVNAQDNEDHVPLHFCSRFGHHDIVKYLLQSDLEVQPHVVNIYGDTPLH LACYNGKFEVAKEIIQISGTESLTKENIFSETAFHSACTYGKSIDLVKFLLDQNVININHQGRDG HTGLHSACYHGHIRLVQFLLDNGADMNLVACDPSRSSGEKDEQTCLMWAYEKGHDAIVTLL KHYKRPQDELPCNEYSQPGGDGSYVSVPSPLGKIKSMTKEKADILLLRAGLPSITFHLQLSEIEF HEIIGSGSFGKVYKGRCRNKIVAIKRYRANTYCSKSDVDMFCREVSILCQLNHPCVIQFVGAC LNDPSQFAIVTQYISGGSLFSLLHEQKR corresponding to amino acids 1-657 of TNI3K_HUMAN (SEQ. ID NO: 396), which also corresponds to amino acids 12-668 of C03950_{—}3_P20 (SEQ. ID NO:222), and a third amino acid sequence being at least 70%, optionally at least 80%, preferably at least 85%, more preferably at least 90% and most preferably at least 95% homologous to a polypeptide having the sequence YGSFVLIYPWTFRRNYSCNTSEGFPLDEPSPFEI (SEQ. ID NO: 372) corresponding to amino acids 669-702 of C03950_{—}3_P20 (SEQ. ID NO:222), wherein said amino acid sequence, second amino acid sequence and third amino acid sequence are contiguous and in a sequential order.

B. An isolated polypeptide encoding for a head of C03950_{—}3_P20 (SEQ. ID NO:222), comprising a polypeptide being at least 70%, optionally at least about 80%, preferably at least about 85%, more preferably at least about 90% and most preferably at least about 95% homologous to the sequence AVRRGLREGGA (SEQ. ID NO: 342) of C03950_{—}3_P20 (SEQ. ID NO:222).

C. An isolated polypeptide encoding for an edge portion of C03950_{—}3_P20 (SEQ. ID NO:222), comprising an amino acid sequence being at least 70%, optionally at least about 80%, preferably at least about 85%, more preferably at least about 90% and most preferably at least about 95% homologous to the sequence YGSFVLIYPWTFRRNYSCNTSEGFPLDEPSPFEI (SEQ. ID NO: 372) of C03950_{—}3_P20 (SEQ. ID NO:222).

### 2. Comparison Report Between C03950_{—}3_P20 (SEQ. ID NO:222) and Q6MZS9_HUMAN (SEQ. ID NO:211):

A. An isolated chimeric polypeptide encoding for C03950_{—}3_P20 (SEQ. ID NO:222), comprising a first amino acid sequence being at least 70%, optionally at least 80%, preferably at least 85%, more preferably at least 90% and most preferably at least 95%, homologous to a polypeptide having the sequence AVRRGLR (SEQ. ID NO: 344) corresponding to amino acids 1-7 of C03950_{—}3_P20 (SEQ. ID NO:222), a second amino acid sequence being at least 90% homologous to EGGAMAAARDPPEVSLREATQRKLRRFSELRGKLVARGEFWDIVAITAADEKQELAYNQQLS EKLKRKELPLGVQYHVFVDPAGAKIGNGGSTLCALQCLEKLYGDKWNSFTILLIHSDEWKKK VSESYVITIERLEDDLQIKEKELTELRNIFGSDEAFSKVNLNYRTENGLSLLHLCCICGGKSHI RTLMLKGLRPSRLTRNGFTALHLAVYKDNAELITSLLHSGADIQQVGYGGLTALHIATIAGHL EAADVLLQHGANVNIQDAVFFTPLHIAAYYGHEQVTRULKFGADVNVSGEVGDRPLHLAS AKGFLNIAKLLMEEGSKADVNAQDNEDHVPLHFCSRFGHHD corresponding to amino acids 14-367 of Q6MZS9_HUMAN (SEQ. ID NO:211), which also corresponds to amino acids 8-361 of C03950_{—}3P20 (SEQ. ID NO:222), a bridging amino acid I corresponding to amino acid 362 of C03950_{—}3_P20 (SEQ. ID NO:222), a third amino acid sequence being at least 90% homologous to VKYLLQSDLEVQPHVVNIYGDTPLHLACYNGKFEVAKEIIQISGTESLTKENIFSETAFESACT YGKSIDLVICFLLDQNVININHQGRDGHTGLHSACYHGHIRLVQFLLDNGADM corresponding to amino acids 369-484 of Q6MZS9_HUMAN (SEQ. ID NO:211), which also corresponds to amino acids 363-478 of C03950_{—}3_P20 (SEQ. ID NO:222), a bridging amino acid N corresponding to amino acid 479 of C03950_{—}3_P20 (SEQ. ID NO:222), a fourth amino acid sequence being at least 90% homologous to LVACDPSRSSGEKDEQTCLMWAYEKGHDANTLLKHYKRPQDELPCNEYSQPGGDGSYVSV PSPLGKEKSMTKEICADILLLRAGLPSHIHLQLSEIEFHETIGSGSFGKVYKGRCRNICIVAIKRYR ANTYCSKSDVDMFCREVSILCQLNHPCVIQFVGACLNDPSQFAIVTQYISGGSLFSLLHEQKR corresponding to amino acids 486-674 of Q6MZS9_HUMAN (SEQ. ID NO:211), which also corresponds to amino acids 480-668 of C03950_{—}3_P20 (SEQ. ID NO:222), and a fifth amino acid sequence being at least 70%, optionally at least 80%, preferably at least 85%, more preferably at least 90% and most preferably at least 95% homologous to a polypeptide having the sequence YGSFVLIYPWTFRRNYSCNTSEGFPLDEPSPFEI (SEQ. ID NO: 372) corresponding to amino acids 669-702 of C03950_{—}3_P20 (SEQ. ID NO:222), wherein said first amino acid sequence, second amino acid sequence, bridging amino acid, third amino acid sequence, bridging amino acid, fourth amino acid sequence and fifth amino acid sequence are contiguous and in a sequential order.

B. An isolated polypeptide encoding for a head of C03950_{—}3_P20 (SEQ. ID NO:222), comprising a polypeptide being at least 70%, optionally at least about 80%, preferably at least about 85%, more preferably at least about 90% and most preferably at least about 95% homologous to the sequence AVRRGLR (SEQ. ID NO: 344) of C03950_{—}3_P20 (SEQ. ID NO:222).

### 3. Comparison Report Between C03950_{—}3_P20 (SEQ. ID NO:222) and NP_{—}057062 (SEQ. ID NO:210):

A. An isolated chimeric polypeptide encoding for C03950_{—}3_P20 (SEQ. ID NO:222), comprising a first amino acid sequence being at least 70%, optionally at least 80%, preferably at least 85%, more preferably at least 90% and most preferably at least 95%, homologous to a polypeptide having the sequence AVRRGLREGGAMAAARDPPEVSLREATQRKLRRFSELRGKLVARGEFWDIVAITAADEKQEL AYNQQLSEKLKRKELPLGVQYHVFVDPAGAKIGNGGSTLCALQCLEKLYGDKWNSFTILLIH S (SEQ. ID NO: 343) corresponding to amino acids 1-125 of C03950_{—}3_P20 (SEQ. ID NO:222), a second amino acid sequence being at least 90% homologous to DEWKKKVSESYVITIERLEDDLQIKEKELTELRNIFGSDEAFSKVNLNYRTENGLSLLHLCCIC GGKKSHIRTLMLKGLRPSRLTRNGFTALHLAVYKDNAELITSLLHSGADIQQVGYGGLTALHI ATIAGHLEAADVLLQHGANVNIQDAVFFTPLHRAYYGHEQVTRILLKFGADVNVSGEVGDR PLBLASAKGFLNIAKLLMEEGSKADVNAQDNEDHVPLHFCSRFGHHDIVKYLLQSDLEVQPH VVNIYGDTPLHLACYNGKFEVAKEIIQISGTESLTKENIFSETAFHSACTYGKSIDLVKFLLDQN VININHQGRDGHTGLHSACYHGHIRLVQFLLDNGADMNLVACDPSRSSGEKDEQTCLMWAY EKGHDAIVTLLKHYKRPQDELPCNEYSQPGGDGSYVSVPSPLGKIKSMTKEKADILLLRAGLP SHFHLQLSEIEFHEIIGSGSFGKVYKGRCRNKIVAIKRYRANTYCSKSDVDMFCREVSILCQLN HPCVIQFVGACLNDPSQFAIVTQYISGGSLFSLLHEQKR corresponding to amino acids 14-556 of NP_{—}057062 (SEQ. ID NO:210), which also corresponds to amino acids 126-668 of C03950_{—}3_P20 (SEQ. ID NO:222), and a third amino acid sequence being at least 70%, optionally at least 80%, preferably at least 85%, more preferably at least 90% and most preferably at least 95% homologous to a polypeptide having the sequence YGSFVLIYPWTFRRNYSCNTSEGFPLDEPSPFEI (SEQ. ID NO: 372) corresponding to amino acids 669-702 of C03950_{—}3_P20 (SEQ. ID NO:222), wherein said first amino acid sequence, second amino acid sequence and third amino acid sequence are contiguous and in a sequential order.

B. An isolated polypeptide encoding for a head of C03950_{—}3_P20 (SEQ. ID NO:222), comprising a polypeptide being at least 70%, optionally at least about 80%, preferably at least about 85%, more preferably at least about 90% and most preferably at least about 95% homologous to the sequence AVRRGLREGGAMAAARDPPEVSLREATQRKLRRFSELRGKLVARGEFWDIVAITAADEKQEL AYNQQLSEKLKRKELPLGVQYHVEVDPAGAKIGNGGSTLCALQCLEKLYGDKWNSFTILLIH S (SEQ. ID NO: 343) of C03950_{—}3_P20 (SEQ. ID NO:222).

The localization of the variant protein was determined according to results from a number of different software programs and analyses, including analyses from SignalP and other specialized programs. The variant protein is believed to be located intracellularly.

Variant protein C03950_{—}3_P20 (SEQ. ID NO:222) also has the following non-silent SNPs (Single Nucleotide Polymorphisms) as listed in Table 106, (given according to their position(s) on the amino acid sequence, with the alternative amino acid(s) listed.

TABLE 106
Amino acid mutations

| SNP position(s) on amino acid | |
|---|---|
| sequence | Alternative amino acid(s) |
| 119 | T -> P |

The variant protein has the following domains, as determined by using InterPro. The domains are described in Table 107:

TABLE 107
InterPro domain(s)

| Domain | | |
|---|---|---|
| description | Analysis type | Position(s) on protein |
| Protein kinase | BlastProDom | 575-668 |
| Ankyrin | FPrintScan | 279-291, 464-476 |
| Ankyrin | HMMPfam | 178-211, 212-244, 245-277, 278-310, |
| | | 311-345, 346-376, 381-414, 416-450, |
| | | 451-483, 493-525 |
| Tyrosine protein | HMMSmart | 575-702 |
| kinase | | |
| Serine | HMMSmart | 575-700 |
| Ankyrin | HMMSmart | 178-208, 212-241, 245-274, 278-307, |
| | | 311-342, 346-377, 381-412, 416-447, |
| | | 451-480, 493-522 |
| Protein kinase | ProfileScan | 575-702 |
| Ankyrin | ProfileScan | 212-244, 245-277, 281-310, 311-343, |
| | | 381-413, 451-483 |
| Ankyrin | ProfileScan | 170-513 |
| Protein kinase | ScanRegExp | 581-602 |

Variant protein C03950_{—}3_P20 (SEQ. ID NO:222) is encoded by the following transcript(s): C03950_{—}3 T18 (SEQ. ID NO:166), for which the coding portion starts at position 3 and ends at position 2108. The transcript also has the following SNPs as listed in Table 108 (given according to their position on the nucleotide sequence, with the alternative nucleic acid listed.

TABLE 108
Nucleic acid SNPs

| SNP position(s) on nucleotide | |
|---|---|
| sequence | Alternative nucleic acid(s) |
| 263 | A -> G |
| 357 | A -> C |

Variant protein C03950_{—}3P21 (SEQ. ID NO:223) according to the present invention is encoded by transcript C03950_{—}3 T19 (SEQ. ID NO:167). One or more alignments to one or more previously published Serine/threonine-protein kinase TNNI3K ((SEQ. ID NO:209) protein sequences are given in the alignment table on the attached CD-ROM. A brief description of the relationship of the variant protein according to the present invention to each such aligned protein is as follows:

1. Comparison report between C03950_{—}3_P21 (SEQ. ID NO:223) and TNI3K_HUMAN (SEQ. ID NO: 396):

A. An isolated chimeric polypeptide encoding for C03950_{—}3_P21 (SEQ. ID NO:223), comprising a first amino acid sequence being at least 70%, optionally at least 80%, preferably at least 85%, more preferably at least 90% and most preferably at least 95%, homologous to a polypeptide having the sequence MGNYKSRPTQTCT (SEQ. ID NO: 346) corresponding to amino acids 1-13 of C03950_{—}3P21 (SEQ. ID NO:223), a second amino acid sequence being at least 90% homologous to DEWKKKVSESYVITERLEDDLQIKEKELTELRNIFGSDEAFSKVNLNYRTENGLSLLHLCCIC GGKKSHIRTLMLKGLRPSRLTRNGFTALHLAVYKDNAELITSLLHSGADIQQVGYGGLTALHI ATIAGBLEAADVLLOHGANVNIQDAVFFTPLHIAAYYGBEQVTRILLKFGADVNVSGEVGDR PLIALASAKGFLNIAKLLMEEGSKADVNAQDNEDHVPLHFCSRFGHEIDIVKYLLQSDLEVQPH VVNIYGDTPLHLACYNGKFEVAKEIIQISGTESLTKENIFSETAFHSACTYGKSIDLVKFLLDQN VININHQGRDGHTGLHSACYHGHIRLVQFLLDNGADMNLVACDPSRSSGEKDEQTCLMWAY EKGHDAFVTLLKHYKRPQDELPCNEYSQPGGDGSYVSVPSPLGKIKSMTKEKADILLLRAGLP SHFHLQLSEIEFHEIIGSGSFGKVYKGRCRNKIVAIKRYRANTYCSKSDVDMECREVSILCQLN HPCVIQFVGACLNDPSQFAIVTQYISGGSLFSLLHEQKR corresponding to amino acids 115-657 of TNI3K_HUMAN (SEQ. ID NO: 396), which also corresponds to amino acids 14-556 of C03950_{—}3_P21 (SEQ. ID NO:223), and a third amino acid sequence being at least 70%, optionally at least 80%, preferably at least 85%, more preferably at least 90% and most preferably at least 95% homologous to a polypeptide having the sequence YGSFVLIYPWTFRRNYSCNTSEGFPLDEPSPFEI (SEQ. ID NO: 372) corresponding to amino acids 557-590 of C03950_{—}3_P21 (SEQ. ID NO:223), wherein said first amino acid sequence, second amino acid sequence and third amino acid sequence are contiguous and in a sequential order.

B. An isolated polypeptide encoding for a head of C03950_{—}3_P21 (SEQ. ID NO:223), comprising a polypeptide being at least 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P21 (SEQ. ID NO:223).

C. An isolated polypeptide encoding for an edge portion of C03950_{—}3_P21 (SEQ. ID NO:223), comprising an amino acid sequence being at least 70%, optionally at least about 80%, preferably at least about 85%, more preferably at least about 90% and most preferably at least about 95% homologous to the sequence YGSFVLIYPWTFRRNYSCNTSEGFPLDEPSPFEI (SEQ. ID NO: 372) of C03950_{—}3_P21 (SEQ. ID NO:223).

2. Comparison report between C03950_{—}3_P21 (SEQ. ID NO:223) and Q6MZS9_HUMAN (SEQ ID NO:211):

A. An isolated chimeric polypeptide encoding for C039503P21 (SEQ. ID NO:223), comprising a first amino acid sequence being at least 70%, optionally at least 80%, preferably at least 85%, more preferably at least 90% and most preferably at least 95%, homologous to a polypeptide having the sequence MGNYKSRPTQTCT (SEQ. ID NO: 346) corresponding to amino acids 1-13 of C03950_{—}3_P21 (SEQ. ID NO:223), a second amino acid sequence being at least 90% homologous to DEWKKKVSESYVITIERLEDDLQIKEKELTELRNIFGSDEAFSKVNLNYRTENGLSLHLCCIC GGKKSHIRTLMLKGLRPSRLTRNGFTALHLAVYKDNAELITSLLHSGADIQQVGYGGLTALHI ATIAGHLEAADVLLQHGANVNIQDAVFFTPLHIAAYYGHEQVTRLLLKFGADVNVSGEVGDR PLHLASAKGFLNIAKLLMEEGSKADVNAQDNEDHVPLHFCSRFGHHD corresponding to amino acids 132-367 of Q6MZS9_HUMAN (SEQ. ID NO:211), which also corresponds to amino acids 14-249 of C03950_{—}3_P21 (SEQ. ID NO:223), a bridging amino acid I corresponding to amino acid 250 of C03950_{—}3_P21 (SEQ. ID NO:223), a third amino acid sequence being at least 90% homologous to VKYLLQSDLEVQPHVVNIYGDTPLHACYNGKFEVAKEIIQISGTESLTKENIFSETAFHSACT YGKSIDLVKFLLDQNVININHQGRDGHTGLHSACYHGHIRLVQFLLDNGADM corresponding to amino acids 369-484 of Q6MZS9 HUMAN (SEQ. ID NO:211), which also corresponds to amino acids 251-366 of C03950_{—}3_P21 (SEQ. ID NO:223), a bridging amino acid N corresponding to amino acid 367 of C03950_{—}3_P21 (SEQ. ID NO:223), a fourth amino acid sequence being at least 90% homologous to LVACDPSRSSGEKDEQTCLMWAYEKGHDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNHPCVIQFVGACLNDPSQFAIVTQYISGGSLFSLLHEQKR corresponding to amino acids 486-674 of Q6MZS9_HUMAN (SEQ. ID NO:211), which also corresponds to amino acids 368-556 of C03950_{—}3_P21 (SEQ. ID NO:223), and a fifth amino acid sequence being at least 70%, optionally at least 80%, preferably at least 85%, more preferably at least 90% and most preferably at least 95% homologous to a polypeptide having the sequence YGSFVLIYPWTFRRNYSCNTSEGFPLDEPSPFEI (SEQ. ID NO: 372) corresponding to amino acids 557-590 of C03950_{—}3_P21 (SEQ. ID NO:223), wherein said first amino acid sequence, second amino acid sequence, bridging amino acid, third amino acid sequence, bridging amino acid, fourth amino acid sequence and fifth amino acid sequence are contiguous and in a sequential order.

3. Comparison report between C03950_{—}3_P21 (SEQ. ID NO:223) and NP_{—}057062 (SEQ. ID NO:210):

A. An isolated chimeric polypeptide encoding for C03950_{—}3_P21 (SEQ. ID NO:223), comprising a first amino acid sequence being at least 90% homologous to MGNYKSRPTQTCTDEWKKKVSESYVITIERLEDDLQIKEKELTELRNIFGSDEAFSKVNLNYR TENGLSLLHLCCICGGKKSHIRTLMLKGLRPSRLTRNGFTALHLAVYKDNAELITSLLHSGADI QQVGYGGLTALHIATIAGHLEAADVILQHGANVNIQDAVFFTPLHEAAYYGREQVTRLLLKF GADVNVSGEVGDRPLHLASAKGFLNIAKLLMEEGSKADVNAQDNEDHVPLHFCSRFGHHDI VKYLLQSDLEVQPHVVNTYGDTPLHLACYNGKFEVAKEIIQISGTESLTKENIFSETAFHSACT YGKSIDLVKFLLDQNVININHQGRDGHTGLHSACYHGHERLVQFLLDNGADMNLVACDPSRS SGEKDEQTCLMWAYEKGHDAIVTLLKHYKRPQDELPCNEYSQPGGDGSYVSVPSPLGKIKSM TKEKADILLLRAGLPSHFHLQLSEIEFHEIIGSGSFGKVYKGRCRNKIVAIKRYRANTYCSKSDV DMFCREVSILCQLNHPCVIQFVGACLNDPSQFAIVTQYISGGSLFSLLHEQKR corresponding to amino acids 1-556 of NP_{—}057062 (SEQ. ID NO:210), which also corresponds to amino acids 1-556 of C03950_{—}3_P21 (SEQ. ID NO:223), and a second amino acid sequence being at least 70%, optionally at least 80%, preferably at least 85%, more preferably at least 90% and most preferably at least 95% homologous to a polypeptide having the sequence YGSFVLIYPWTFRRNYSCNTSEGFPLDEPSPFEI (SEQ. ID NO: 372) corresponding to amino acids 557-590 of C03950_{—}3_P21 (SEQ. ID NO:223), wherein said first amino acid sequence and second amino acid sequence are contiguous and in a sequential order.

The localization of the variant protein was determined according to results from a number of different software programs and analyses, including analyses from SignalP and other specialized programs. The variant protein is believed to be located intracellularly.

The variant protein has the following domains, as determined by using InterPro. The domains are described in Table 109:

TABLE 109
InterPro domain(s)

| Domain | | |
|---|---|---|
| description | Analysis type | Position(s) on protein |
| Protein kinase | BlastProDom | 463-556 |
| Ankyrin | FPrintScan | 167-179, 352-364 |
| Ankyrin | HMMPfam | 66-99, 100-132, 133-165, 166-198, |
| | | 199-233, 234-264, 269-302, 304-338, |
| | | 339-371, 381-413 |
| Tyrosine protein | HMMSmart | 463-590 |
| kinase | | |
| Serine | HMMSmart | 463-588 |
| Ankyrin | HMMSmart | 66-96, 100-129, 133-162, 166-195, |
| | | 199-230, 234-265, 269-300, 304-335, |
| | | 339-368, 381-410 |
| Protein kinase | ProfileScan | 463-590 |
| Ankyrin | ProfileScan | 100-132, 133-165, 169-198, 199-231, |
| | | 269-301, 339-371 |
| Ankyrin | ProfileScan | 58-401 |
| Protein kinase | ScanRegExp | 469-490 |

Variant protein C03950_{—}3 P21 (SEQ. ID NO:223) is encoded by the following transcript(s): C03950_{—}3_T19 (SEQ. ID NO:167), for which the coding portion starts at position 389 and ends at position 2158.

Variant protein C03950_{—}3_P23 (SEQ. ID NO:224) according to the present invention is encoded by transcript C03950_{—}3 T21 (SEQ. ID NO:168). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

1. Comparison report between C03950_{—}3_P23 (SEQ. ID NO:224) and Q6MZS9_HUMAN (SEQ NO:211):

A. An isolated chimeric polypeptide encoding for C03950_{—}3_P23 (SEQ. ID NO:224), comprising a first amino acid sequence being at least 70%, optionally at least 80%, preferably at least 85%, more preferably at least 90% and most preferably at least 95%, homologous to a polypeptide having the sequence MGNYKSRPTQTCT (SEQ. ID NO: 346) corresponding to amino acids 1-13 of C03950_{—}3_P23 (SEQ. ID NO:224), a second amino acid sequence being at least 90% homologous to DEWKKKVSESYVITIERLEDDLQIKEKELTELRNIFGSDEAFSKVNLNYRTENGLSLLBLCCIC GGKKSHIRTLMLKGLRPSRLTRNGFTALHELAVYKDNAELITSLLHSGADIQQVGYGGLTALHI ATIAGHLEAADVLLQHGANVNIQDAVFFTPLHIAAYYGHEQVTRLLLKFGADVNVSGEVGDR PLHLASAKGFLNIAKLLMEEGSKADVNAQDNEDHVPLHFCSRFGHHD corresponding to amino acids 132-367 of Q6MZS9_HUMAN (SEQ. ID NO:211), which also corresponds to amino acids 14-249 of C03950_{—}3_P23 (SEQ. ID NO:224), a bridging amino acid I corresponding to amino acid 250 of C03950_{—}3_P23 (SEQ. ID NO:224), a third amino acid sequence being at least 90% homologous to VKYLLQSDLEVQPHVVNIYGDTPLHLACYNGKFEVAKEIIQISGTESLTKENIFSETAFHSACT YGKSIDLVKFLLDQNVININHQGRDGHTGLHSACYHGHIRLVQFLLDNGADM corresponding to amino acids 369-484 of Q6MZS9_HUMAN (SEQ. ID NO:211), which also corresponds to amino acids 251-366 of C03950_{—}3_P23 (SEQ. ID NO:224), a bridging amino acid N corresponding to amino acid 367 of C03950_{—}3_P23 (SEQ. ID NO:224), a fourth amino acid sequence being at least 90% homologous to LVACDPSRSSGEKDEQTCLMWAYEKGHDAIVTLLKHYKRPQDELPCNEYSQPGGDGSYVSV PSPLGKIKSMTKEKADILLLRAGLPSHFHLQLSEIEFHEIIGSG corresponding to amino acids 486-590 of Q6MZS9_HUMAN (SEQ. ID NO:211), which also corresponds to amino acids 368-472 of C03950_{—}3_P23 (SEQ. ID NO:224), and a fifth amino acid sequence being at least 70%, optionally at least 80%, preferably at least 85%, more preferably at least 90% and most preferably at least 95% homologous to a polypeptide having the sequence NLK (SEQ. ID NO: 378) corresponding to amino acids 473-475 of C03950_{—}3_P23 (SEQ. ID NO:224), wherein said first amino acid sequence, second amino acid sequence, bridging amino acid, third amino acid sequence, bridging amino acid, fourth amino acid sequence and fifth amino acid sequence are contiguous and in a sequential order.

B. An isolated polypeptide encoding for a head of C03950_{—}3_P23 (SEQ. ID NO:224), comprising a polypeptide being at least 70%, optionally at least about 80%, preferably at least about 85%, more preferably at least about 90% and most preferably at least about 95% homologous to the sequence MGNYKSRPTQTCT (SEQ. ID NO: 346) of C03950_{—}3_P23 (SEQ. ID NO:224).

C. An isolated polypeptide encoding for an edge portion of C03950_{—}3_P23 (SEQ. ID NO:224), comprising an amino acid sequence being at least 70%, optionally at least about 80%, preferably at least about 85%, more preferably at least about 90% and most preferably at least about 95% homologous to the sequence NLK (SEQ. ID NO: 378) of C03950_{—}3_P23 (SEQ. ID NO:224).

2. Comparison report between C03950_{—}3_P23 (SEQ. ID NO:224) and NP_{—}057062 (SEQ. ID NO:210):

A. An isolated chimeric polypeptide encoding for C03950_{—}3_P23 (SEQ. ID NO:224), comprising a first amino acid sequence being at least 90% homologous to MGNYKSRPTQTCTDEWKKKVSESYVITIERLEDDLQIKEKELTELRNIFGSDEAFSKVNLNYR TENGLSLLHLCCICGGKKSHIRTLMLKGLRPSRLTRNGFTALHLAVYKDNAELITSLLHSGADI QQVGYGGLTALHIATIAGHLEAADVLLQHGANVNIQDAVFFTPLHIAAYYGHEQVTRLLLKF GADVNVSGEVGDRPLHLASAKGFLNIAKLLMEEGSKADVNAQDNEDHVPLHFCSRFGHHDI VKYLLQSDLEVQPHVVNIYGDTPLELACYNGKFEVAKEIIQSGTESLTKENIFSETAMSACT YGKSIDLVKFLLDQNVININHQGRDGHTGLHSACYHGHERLVQFLLDNGADMNLVACDPSRS SGEKDEQTCLMWAYEKGHDAIVTLLKHYKRPQDELPCNEYSQPGGDGSYVSVPSPLGKIKSM TKEKADILLLRAGLPSHFHLQLSEIEFHEIIGSG corresponding to amino acids 1-472 of NP_{—}057062 (SEQ. ID NO:210), which also corresponds to amino acids 1-472 of C03950_{—}3_P23 (SEQ. ID NO:224), and a second amino acid sequence being at least 70%, optionally at least 80%, preferably at least 85%, more preferably at least 90% and most preferably at least 95% homologous to a polypeptide having the sequence NLK (SEQ. ID NO: 378) corresponding to amino acids 473-475 of C03950_{—}3_P23 (SEQ. ID NO:224), wherein said first amino acid sequence and second amino acid sequence are contiguous and in a sequential order.

The localization of the variant protein was determined according to results from a number of different software programs and analyses, including analyses from SignalP and other specialized programs. The variant protein is believed to be located intracellularly.

The variant protein has the following domains, as determined by using InterPro. The domains are described in Table 110:

TABLE 110
InterPro domain(s)

| Domain | | |
|---|---|---|
| description | Analysis type | Position(s) on protein |
| Ankyrin | FPrintScan | 167-179, 352-364 |
| Ankyrin | HMMPfam | 66-99, 100-132, 133-165, 166-198, |
| | | 199-233, 234-264, 269-302, 304-338, |
| | | 339-371, 381-413 |
| Ankyrin | HMMSmart | 66-96, 100-129, 133-162, 166-195, |
| | | 199-230, 234-265, 269-300, 304-335, |
| | | 339-368, 381-410 |
| Ankyrin | ProfileScan | 100-132, 133-165, 169-198, 199-231, |
| | | 269-301, 339-371 |
| Ankyrin | ProfileScan | 58-401 |

Variant protein C03950_{—}3_P23 (SEQ. ID NO:224) is encoded by the following transcript(s): C03950_{—}3 T21 (SEQ. ID NO:168), for which the coding portion starts at position 389 and ends at position 1813.

Variant protein C03950_{—}3 P24 (SEQ. ID NO:225) according to the present invention is encoded by transcript C03950_{—}3_T22 (SEQ. ID NO:169).

The localization of the variant protein was determined according to results from a number of different software programs and analyses, including analyses from SignalP and other specialized programs. The variant protein is believed to be located intracellularly.

The variant protein has the following domains, as determined by using InterPro. The domains are described in Table 111:

TABLE 111
InterPro domain(s)

| Domain description | Analysis type | Position(s) on protein |
|---|---|---|
| Protein kinase | BlastProDom | 46-84 |
| Protein kinase | ProfileScan | 1-142 |

Variant protein C03950_{—}3_P24 (SEQ. ID NO:225) is encoded by the following transcript(s): C03950_{—}3_T322 (SEQ. ID NO:169), for which the coding portion starts at position 457 and ends at position 1218.

Variant protein C03950_{—}3_P25 (SEQ. ID NO:226) according to the present invention is encoded by transcript C03950_{—}3_T23 (SEQ. ID NO:170).

The localization of the variant protein was determined according to results from a number of different software programs and analyses, including analyses from SignalP and other specialized programs. The variant protein is believed to be located intracellularly.

The variant protein has the following domains, as determined by using InterPro. The domains are described in Table 112:

TABLE 112
InterPro domain(s)

| Domain description | Analysis type | Position(s) on protein |
|---|---|---|
| Protein kinase | BlastProDom | 46-84 |
| Protein kinase | ProfileScan | 1-142 |

Variant protein C03950_{—}3 P25 (SEQ. ID NO:226) is encoded by the following transcript(s): C03950_{—}3_T23 (SEQ. ID NO:170), for which the coding portion starts at position 457 and ends at position 1155.

Variant protein C03950_{—}3_P28 (SEQ. ID NO:227) according to the present invention is encoded by transcript C03950_{—}3_T2 (SEQ. ID NO:156). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

1. Comparison report between C03950_{—}3_P28 (SEQ. ID NO:227) and TNI3K HUMAN (SEQ. ID NO: 396):

A. An isolated chimeric polypeptide encoding for C03950_{—}3_P28 (SEQ. ID NO:227), comprising a first amino acid sequence being at least 90% homologous to MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFITLLIHSDEWKKKVSES YVITEERLEDDLQIKEKELTELRNIFGSDEAFSKVNLNYRTENGLSLLHLCCICGGKKSHERTLM LKGLRPSRLTRNGFTALHLAVYKDNAELITSLLHSGADIQQVGYGGLTALHIATIAGHLEAAD VLLQHGANVNIQDAVFFTPLHIAAYYGHEQVTRLLLKFGADVNVSGEVGDRPLHLASAKGFL NIAKLLMEEGSKADVNAQDNEDHVPLHFCSRFGHHDIVKYLLQSDLEVQPHVVNIYGDTPLH LACYNGKFEVAKEIIQISGTESLTKENIFSETAFHSACTYGKSIDLVKFLLDQNVININHQGRDG HTGLHSACYHGHIRLVQFLLDNGADMNLVACDPSRSSGEKDEQTCLMWAYEKGHDAIVTLL KHYKRPQDELPCNEYSQPGGDGSYVSVPSPLGKIKSMTKEKADILLLRAGLPSHFHLQLSEIEF HEIIGSGSFGKVYKGRCRNKIVAIKRYRANTYCSKSDVDMFCREVSILCQLNHPCVIQFVGAC LNDPSQFAIVTQYISGGSLFSLLHEQKRILDLQSKLIIAVDVAKGMEYLHNLTQPIIHRDLN corresponding to amino acids 1-691 of TNI3K_HUMAN (SEQ. ID NO: 396), which also corresponds to amino acids 1-691 of C03950_{—}3 P28 (SEQ. ID NO:227), a second amino acid sequence being at least 70%, optionally at least 80%, preferably at least 85%, more preferably at least 90% and most preferably at least 95%, homologous to a polypeptide having the sequence RSAITSRWITHSICIWRGAHYFNREECNFRCMLTSAILK corresponding to amino acids 692-731 of C03950_{—}3_P28 (SEQ. ID NO:227), and a third amino acid sequence being at least 90% homologous to ESRFLQSLDEDNMTKQPGNLRWMAPEVFTQCTRYTIKADVFSYALCLWEILTGEIPFAHLKPA AAAADMAYHHIRPPIGYSIPKPISSLLIRGWNACPEGRPEFSEVVMKLEECLCNIELMSPASSNS SGSLSPSSSSDCLVNRGGPGRSHVAALRSRFELEYALNARSYAALSQSAGQYSSQGLSLEEMK RSLQYTPIDKYGYVSDPMSSMHFHSCRNSSSFEDSS corresponding to amino acids 710-936 of TNI3K_HUMAN (SEQ. ID NO: 396), which also corresponds to amino acids 732-958 of C03950_{—}3_P28 (SEQ. ID NO:227), wherein said first amino acid sequence, second amino acid sequence and third amino acid sequence are contiguous and in a sequential order.

2. Comparison report between C03950_{—}3_P28 (SEQ. ID NO:227) and NP_{—}057062 (SEQ. ID NO:210):

A. An isolated chimeric polypeptide encoding for C03950_{—}3_P28 (SEQ. ID NO:227), comprising a first amino acid sequence being at least 70%, optionally at least 80%, preferably at least 85%, more preferably at least 90% and most preferably at least 95%, homologous to a polypeptide having the sequence MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHS (SEQ. ID NO: 379) corresponding to amino acids 1-114 of C03950_{—}3_P28 (SEQ. ID NO:227), a second amino acid sequence being at least 90% homologous to DEWKKKVSESYVITIERLEDDLQIKEKELTELRNIFGSDEAFSKVNLNYRTENGLSLLHLCCIC GGKKSHIRTLMLKGLRPSRLTRNGFTALHLAVYKDNAELITSLLHSGADIQQVGYGGLTALHI ATIAGHLEAADVLLQHGANVNIQDAVFFTPLHIAAYYGHEQVTRLLLKFGADVNVSGEVGDR PLHLASAKGFLNIAKLLMEEGSKADVNAQDNEDHVPLHFCSRFGHBDIVKYLLQSDLEVQPH VVNIYGDTPLHACYNGKFEVAKEIIQISGTESLTKENIFSETAFHSACTYGKSIDLVKFILDQN VININHQGRDGHTGLHSACYHGHIRLVQFLLDNGADMNLVACDPSRSSGEKDEQTCLMWAY EKGHDAIVTLLKHYKRPQDELPCNEYSQPGGDGSYVSVPSPLGKIKSMTKEKADILLLRAGLP SHFHLQLSEIEFHEIIGSGSFGKVYKGRCRNKIVAIKRYRANTYCSKSDVDMFCREVSILCQLN HPCVIQFVGACLNDPSQFAIVTQYISGGSLFSLLHEQKRILDLQSKLIIAVDVAKGMEYLHNLT QPIIHRDLN corresponding to amino acids 14-590 of NP_{—}057062 (SEQ. ID NO:210), which also corresponds to amino acids 115-691 of C03950_{—}3_P28 (SEQ. ID NO:227), a third amino acid sequence being at least 70%, optionally at least 80%, preferably at least 85%, more preferably at least 90% and most preferably at least 95%, homologous to a polypeptide having the sequence RSAITSRIWITHSICIWRGAHYFNREECNFRCMLTSAILK corresponding to amino acids 692-731 of C03950_{—}3_P28 (SEQ. ID NO:227), and a fourth amino acid sequence being at least 90% homologous to ESRFLQSLDEDNMTKQPGNLRWMAPEVFTQCTRYTIKADVFSYALCLWEILTGEIPFAHLKPA AAAADMAYHHIRPPIGYSIPKPISSLLIRGWNACPEGRPEFSEVVMKLEECLCNIELMSPASSNS SGSLSPSSSSDCLVNRGGPGRSHVAALRSRFELEYALNARSYAALSQSAGQYSSQGLSLEEMK RSLQYTPIDKYGYVSDPMSSMHFHSCRNSSSFEDSS corresponding to amino acids 609-835 of NP_{—}057062 (SEQ. ID NO:210), which also corresponds to amino acids 732-958 of C03950_{—}3_P28 (SEQ. ID NO:227), wherein said first amino acid sequence, second amino acid sequence, third amino acid sequence and fourth amino acid sequence are contiguous and in a sequential order.

B. An isolated polypeptide encoding for a head of C03950_{—}3_P28 (SEQ. ID NO:227), comprising a polypeptide being at least 70%, optionally at least about 80%, preferably at least about 85%, more preferably at least about 90% and most preferably at least about 95% homologous to the sequence MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHS (SEQ. ID NO: 379) of C03950_{—}3_P28 (SEQ. ID NO:227).

3. Comparison report between C03950_{—}3_P28 (SEQ. ID NO:227) and Q9Y2V6_HUMAN (SEQ. ID NO:210):

A. An isolated chimeric polypeptide encoding for C03950_{—}3_P28 (SEQ. ID NO:227), comprising a first amino acid sequence being at least 70%, optionally at least 80%, preferably at least 85%, more preferably at least 90% and most preferably at least 95%, homologous to a polypeptide having the sequence MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHS (SEQ. ID NO: 379) corresponding to amino acids 1-114 of C039503P28 (SEQ. ID NO:227), a second amino acid sequence being at least 90% homologous to DEWKKKVSESYVITIERLEDDLQEKEKELTELRNLEGSDEAFSKVNLNYRTENGLSLLHLCCIC GGKKSHIRTLMLKGLRPSRLTRNGFTALBLAVYKDNAELITSLLHSGADIQQVGYGGLTALHI ATIAGHLEAADVLLQHGANVNIQDAVFFTPLHIAAYYGBEQVTRLLLKFGADVNVSGEVGDR PLHLASAKGFLNIAKLLMEEGSKADVNAQDNEDHVPLHFCSRFGHBDIVKYLLQSDLEVQPH VVNIYGDTPLHLACYNGKFEVAKEIIQISGTESLTKENIFSETAFHSACTYGKSIDLVKFLLDQN VININHQGRDGHTGLHSACYHGHIRLVQFLLDNGADMNLVACDPSRSSGEKDEQTCLMWAY EKGHDAIVTLLKHYKRPQDELPCNEYSQPGGDGSYVSVPSPLGKIKSMTKEKADILLLRAGLP SHFHLQLSEIEFHELIGSGSFGKVYKGRCRNKIVAIKRYRANTYCSKSDVDMFCREVSILCQLN HPCVIQFVGACLNDPSQFAIVTQYISGGSLFSLLHEQKRELDLQSKLIIAVDVAKGMBYLHNLT QPIIHRDLN corresponding to amino acids 14-590 of Q9Y2V6_HUMAN (SEQ. ID NO:210), which also corresponds to amino acids 115-691 of C03950_{—}3P28 (SEQ. ID NO:227), a third amino acid sequence being at least 70%, optionally at least 80%, preferably at least 85%, more preferably at least 90% and most preferably at least 95%, homologous to a polypeptide having the sequence RSAITSRIWITHSICIWRGAHYFNREECNFRCMLTSAILK corresponding to amino acids 692-731 of C03950_{—}3_P28 (SEQ. ID NO:227), and a fourth amino acid sequence being at least 90% homologous to ESRFLQSLDEDNMTKQPGNLRWMAPEVFTQCTRYTIKADVFSYALCLWEILTGEIPFAHLKPA AAAADMAYHHIRPPIGYSIPKPISSLLIRGWNACPEGRPEFSEVVMKLEECLCNIELMSPASSNS SGSLSPSSSSDCLVNRGGPGRSHVAALRSRFELEYALNARSYAALSQSAGQYSSQGLSLEEMK RSLQYTPIDKYGYVSDPMSSMHFHSCRNSSSFEDSS corresponding to amino acids 609-835 of Q9Y2V6_HUMAN (SEQ. ID NO:210), which also corresponds to amino acids 732-958 of C03950_{—}3P28 (SEQ. ID NO:227), wherein said first amino acid sequence, second amino acid sequence, third amino acid sequence and fourth amino acid sequence are contiguous and in a sequential order.

4. Comparison report between C03950_{—}3_P28 (SEQ. ID NO:227) and Q6MZS9_HUMAN (SEQ. ID NO:211):

A. An isolated chimeric polypeptide encoding for C03950_{—}3_P28 (SEQ. ID NO:227), comprising a first amino acid sequence being at least 90% homologous to MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHSDEWKKKVSES YVITIERLEDDLQIKEKELTELRNIFGSDEAFSKVNLNYRTENGLSLLHLCCICGGKKSHIRTLM LKGLRPSRLTRNGFTALHLAVYKDNAELITSLLHSGADIQQVGYGGLTALHIATIAGHLEAAD VLLQHGANVNTQDAVFFTPLHIAAYYGHEQVTRLLLKFGADVNVSGEVGDRPLHLASAKGFL NIAKLLMEEGSKADVNAQDNEDHVPLHFCSRFGHED corresponding to amino acids 18-367 of Q6MZS9_HUMAN (SEQ. ID NO:211), which also corresponds to amino acids 1-350 of C03950_{—}3_P28 (SEQ. ID NO:227), a bridging amino acid I corresponding to amino acid 351 of C03950_{—}3_P28 (SEQ. ID NO:227), a second amino acid sequence being at least 90% homologous to VKYLLQSDLEVQPHVVNIYGDTPLHLACYNGKFEVAKEIIQISGTESLTKENIFSETAFHSACT YGKSIDLVKFLLDQNVININHQGRDGHTGLHSACYHGHIRLVQFLLDNGADM corresponding to amino acids 369-484 of Q6MZS9HUMAN (SEQ. ID NO:211), which also corresponds to amino acids 352-467 of C03950_{—}3_P28 (SEQ. ID NO:227), a bridging amino acid N corresponding to amino acid 468 of C03950_{—}3_P28 (SEQ. ID NO:227), a third amino acid sequence being at least 90% homologous to LVACDPSRSSGEKDEQTCLMWAYEKGHDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNHPCVIQFVGACLNDPSQFAIVTQYISGGSLFSLLHEQKRI LDLQSKLIIAVDVAKGMEYLHNLTQPIIHRDLNR corresponding to amino acids 486-709 of Q6MZS9_HUMAN (SEQ. ID NO:211), which also corresponds to amino acids 469-692 of C039503_P28 (SEQ. ID NO:227), and a fourth amino acid sequence being at least 70%, optionally at least 80%, preferably at least 85%, more preferably at least 90% and most preferably at least 95% homologous to a polypeptide having the sequence SAITSRIWITHSICIWRGAHYFNREECNFRCMLTSAILKESRFLQSLDEDNMTKQPGNLRWMA PEVFTQCTRYTTKADVFSYALCLWEILTGEIPFAHLKPAAAAADMAYHHIRPPIGYSlPKPISSL LIRGWNACPEGRPEFSEVVMKLEECLCNIELMSPASSNSSGSLSPSSSSDCLVNRGGPGRSHVA ALRSRFELEYALNARSYAALSQSAGQYSSQGLSLEEMKRSLQYTPIDKYGYVSDPMSSMHFH SCRNSSSFEDSS (SEQ. ID NO: 345) corresponding to amino acids 693-958 of C03950_{—}3_P28 (SEQ. ID NO:227), wherein said, first amino acid sequence, bridging amino acid, second amino acid sequence, bridging amino acid, third amino acid sequence and fourth amino acid sequence are contiguous and in a sequential order.

B. An isolated polypeptide encoding for an edge portion of C03950_{—}3_P28 (SEQ. ID NO:227), comprising an amino acid sequence being at least 70%, optionally at least about 80%, preferably at least about 85%, more preferably at least about 90% and most preferably at least about 95% homologous to the sequence SAITSRIWITHSICIWRGAHYFNREECNFRCMLTSAILKESRFLQSLDEDNMTKQPGNLRWMA PEVFTQCTRYTIKADVFSYALCLWEILTGEIPFAHLKPAAAAADMAYHHIRPPIGYSTPKPISSL LIRGWNACPEGRPEFSEVVMKLEECLCNIELMSPASSNSSGSLSPSSSSDCLVNRGGPGRSHVA ALRSRFELEYALNARSYAALSQSAGQYSSQGLSLEEMKRSLQYTPEDKYGYVSDPMSSMHTH SCRNSSSFEDSS (SEQ. ID NO: 345) of C03950_{—}3_P28 (SEQ. ID NO:227).

The localization of the variant protein was determined according to results from a number of different software programs and analyses, including analyses from SignalP and other specialized programs. The variant protein is believed to be located intracellularly.

Variant protein C03950_{—}3_P28 (SEQ. ID NO:227) also has the following non-silent SNPs (Single Nucleotide Polimorphisms) as listed in Table 113, (given according to their position(s) on the amino acid sequence, with the alternative amino acid(s) listed.

TABLE 113
Amino acid mutations

| SNP position(s) on amino acid | |
|---|---|
| sequence | Alternative amino acid(s) |
| 108 | T -> P |

The variant protein has the following domains, as determined by using InterPro. The domains are described in Table 114:

TABLE 114
InterPro domain(s)

| Domain | | |
|---|---|---|
| description | Analysis type | Position(s) on protein |
| Protein kinase | BlastProDom | 564-788 |
| Ankyrin | FPrintScan | 268-280, 453-465 |
| Ankyrin | HMMPfam | 167-200, 201-233, 234-266, 267-299, |
| | | 300-334, 335-365, 370-403, 405-439, |
| | | 440-472, 482-514 |
| Protein kinase | HMMPfam | 564-842 |
| Tyrosine protein | HMMSmart | 564-842 |
| kinase | | |
| Serine | HMMSmart | 564-846 |
| Ankyrin | HMMSmart | 167-197, 201-230, 234-263, 267-296, |
| | | 300-331, 335-366, 370-401, 405-436, |
| | | 440-469, 482-511 |
| Protein kinase | ProfileScan | 564-846 |
| Ankyrin | ProfileScan | 201-233, 234-266, 270-299, 300-332, |
| | | 370-402, 440-472 |
| Ankyrin | ProfileScan | 159-502 |
| Protein kinase | ScanRegExp | 570-591 |

Variant protein C03950_{—}3_P28 (SEQ. ID NO:227) is encoded by the following transcript(s): C03950 3_T2 (SEQ. ID NO:156), for which the coding portion starts at position 36 and ends at position 2909. The transcript also has the following SNPs as listed in Table 115 (given according to their position on the nucleotide sequence, with the alternative nucleic acid listed.

TABLE 115
Nucleic acid SNPs

| SNP position(s) on nucleotide | |
|---|---|
| sequence | Alternative nucleic acid(s) |
| 263 | A -> G |
| 357 | A -> C |

Variant protein C03950_{—}3 P31 (SEQ. ID NO:228) according to the present invention is encoded by transcript C03950_{—}3 T10 (SEQ. ID NO:161). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

1. Comparison report between C03950_{—}3_P31 (SEQ. ID NO:228) and TNI3K_HUMAN (SEQ. ID NO: 396):

A. An isolated chimeric polypeptide encoding for C03950_{—}3_P31 (SEQ. ID NO:228), comprising a first amino acid sequence being at least 90% homologous to MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHSDEWKKKVSES YVITIERLEDDLQIKEKELTELRNIFGSDEAFSKVNLNYRTENGLSLLHLCCICGGKKSHIRTLM LKGLRPSRLTRNGFTALHLAVYKDNAELITSLLHSGADIQQVGYGGLTALHIATIAGHLEAAD VLLQHGANVNTQDAVFFTPLHIAAYYGHEQVTRLLLKFGADVNVSGEVGDRPLHLASAKGFL NIAKLLMEEGSKADVNAQDNEDHVPLHFCSRFGHHDIVKYLLQSDLEVQPHVVNIYGDTPLH LACYNGKFEVAKEIIQISGTESLTKENIFSETAFHSACTYGKSIDLVKFLLDQNVININHQGRDG HTGLHSACYHGRERLVQFLLDNGADMNLVACDPSRSSGEKDEQTCLMWAYEKGHDAIVTLL KHYKRPQDELPCNEYSQPGGDGSYVSVPSPLGKIKSMTKEKADILLLRAGLPSHFHLQLSEIEF HEIIGSGSFGKVYKGRCRNKIVAIKRYRANTYCSKSDVDMFCREVSILCQLNIPCVIQFVGAC LNDPSQFAIVTQYISGGSLFSLLHEQKRILDLQSKLIIAVDVAKGMEYLFINLTQPIIHRDLNSHN LLYEDGHAVVADFGESRFLQSLDEDNMTKQPGNLRWMAPEVFTQCTRYTIKADVFSYALCL WEILTGEIPFAHLKPAAAAADMAYHHIRPPIGYSIPKPISSLLIRGWNACPE corresponding to amino acids 1-808 of TNI3K_HUMAN (SEQ. ID NO: 396), which also corresponds to amino acids 1-808 of C03950_{—}3_P31 (SEQ. ID NO:228), and a second amino acid sequence being at least 70%, optionally at least 80%, preferably at least 85%, more preferably at least 90% and most preferably at least 95% homologous to a polypeptide having the sequence AKSRPSHYPVSSVYTETLKKKNEDRFGMWIEYLRR (SEQ. ID NO: 356) corresponding to amino acids 809-843 of C03950_{—}3_P31 (SEQ. ID NO:228), wherein said first amino acid sequence and second amino acid sequence are contiguous and in a sequential order.

B. An isolated polypeptide encoding for an edge portion of C03950_{—}3_P31 (SEQ. ID NO:228), comprising an amino acid sequence being at least 70%, optionally at least about 80%, preferably at least about 85%, more preferably at least about 90% and most preferably at least about 95% homologous to the sequence AKSRPSHYPVSSVYTETLKKKNEDRFGMWIEYLRR (SEQ. ID NO: 356) of C03950_{—}3_P31 (SEQ. ID NO:228).

2. Comparison report between C03950_{—}3_P31 (SEQ. ID NO:228) and Q6MZS9_HUMAN (SEQ. ID NO:211):

A. An isolated chimeric polypeptide encoding for C03950_{—}3 P31 (SEQ. ID NO:228), comprising a first amino acid sequence being at least 90% homologous to MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHSDEWKKKVSES YVITIERLEDDLQIKEKELTELRNIFGSDEAFSKVNLNYRTENGLSLLHLCCICGGKKSHIRTLM LKGLRPSRLTRNGFTALHLAVYKDNAELITSLLHSGADIQQVGYGGLTALHIATIAGHLEAAD VLLQHGANVNIQDAVFFTPLHLAAYYGREQVTRLLLKFGADVNVSGEVGDRPLBLASAKGFL NIAKLLMEEGSKADVNAQDNEDHVPLHFCSRFGHHD corresponding to amino acids 18-367 of Q6MZS9_HUMAN (SEQ. ID NO:211), which also corresponds to amino acids 1-350 of C03950_{—}3_P31 (SEQ. ID NO:228), a bridging amino acid I corresponding to amino acid 351 of C03950_{—}3_P31 (SEQ. ID NO:228), a second amino acid sequence being at least 90% homologous to VKYLLQSDLEVQPHVVNIYGDTPLHLACYNGKFEVAKEIIQISGTESLTKENIFSETAFHSACT YGKSIDLVKFLLDQNVININHQGRDGHTGLHSACYHGHIRLVQFLLDNGADM corresponding to amino acids 369-484 of Q6MZS9_HUMAN (SEQ. ID NO:211), which also corresponds to amino acids 352-467 of C03950_{—}3_P31 (SEQ. ID NO:228), a bridging amino acid N corresponding to amino acid 468 of C03950_{—}3_P31 (SEQ. ID NO:228), a third amino acid sequence being at least 90% homologous to LVACDPSRSSGEKDEQTCLMWAYEKGHDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNHPCVIQFVGACLNDPSQFAIVTQYISGGSLFSLLHEQKRI LDLQSKLIIAVDVAKGMEYLHNLTQPIIHRDLN corresponding to amino acids 486-708 of Q6MZS9 HUMAN (SEQ. ID NO:211), which also corresponds to amino acids 469-691 of C03950_{—}3_P31 (SEQ. ID NO:228), and a fourth amino acid sequence being at least 70%, optionally at least 80%, preferably at least 85%, more preferably at least 90% and most preferably at least 95% homologous to a polypeptide having the sequence SHNILLYEDGHAVVADFGESRFLQSLDEDNMTKQPGNLRWMAPEVFTQCTRYTIKADVFSYA LCLWEILTGEIPFAHLKPAAAAADMAYHHIRPPIGYSIPKPISSLLIRGWNACPEAKSRPSHYPV SSVYTETLKKKNEDRFGMWIEYLRR (SEQ. ID NO: 358) corresponding to amino acids 692-843 of C03950_{—}3_P31 (SEQ. ID NO:228), wherein said, first amino acid sequence, bridging amino acid, second amino acid sequence, bridging amino acid, third amino acid sequence and fourth amino acid sequence are contiguous and in a sequential order.

B. An isolated polypeptide encoding for an edge portion of C03950_{—}3_P31 (SEQ. ID NO:228), comprising an amino acid sequence being at least 70%, optionally at least about 80%, preferably at least about 85%, more preferably at least about 90% and most preferably at least about 95% homologous to the sequence SHNILLYEDGHAVVADFGESRFLQSLDEDNMTKQPGNLRWMAPEVFTQCTRYTIKADVFSYA LCLWEILTGEIPFAHLKPAAAAADMAYHHITRPPIGYSIPKPISSLLIRGWNACPEAKSRPSHYPV SSVYTETLKKXNEDRFGMWIEYLRR (SEQ. ID NO: 358) of C03950_{—}3_P31 (SEQ. ID NO:228).

3. Comparison report between C03950_{—}3_P31 (SEQ. ID NO:228) and NP_{—}057062 (SEQ. ID NO:210):

A. An isolated chimeric polypeptide encoding for C03950_{—}3_P31 (SEQ. ID NO:228), comprising a first amino acid sequence being at least 70%, optionally at least 80%, preferably at least 85%, more preferably at least 90% and most preferably at least 95%, homologous to a polypeptide having the sequence MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHS (SEQ. ID NO: 379) corresponding to amino acids 1-114 of C03950_{—}3_P31 (SEQ. ID NO:228), a second amino acid sequence being at least 90% homologous to DEWKKKVSESYVITIERLEDDLQIKEKELTELRNIFGSDEAFSKVNLNYRTENGLSLLHLCCIC GGKKSHLRTLMLKGLRPSRLTRNGFTALHLAVYKDNAELITSLLHSGADIQQVGYGGLTALHI ATIAGHLEAADVLLQHGANVNIQDAVFFTPLHIAAYYGHEQVTRLLLKFGADVNVSGEVGDR PLHLASAKGFLNIAKLLMEEGSKADVNAQDNEDHVPLHFCSRFGHHDIVKYLLQSDLEVQPH VVNIYGDTPLHLACYNGKFEVAKEIIQISGTESLTKENIFSETAFHSACTYGKSIDLVKFLLDQN VININHQGRDGHTGLHSACYHGHIRLVQFLLDNGADMNLVACDPSRSSGEKDEQTCLMWAY EKGHDAIVTLLKHYKRPQDELPCNEYSQPGGDGSYVSVPSPLGKIKSMTKEKADILLLRAGLP SHFHLQLSEIEFHEIIGSGSFGKVYKGRCRNKIVAIKRYRANTYCSKSDVDMFCREVSILCQLN HPCVIQFVGACLNDPSQFAIVTQYISGGSLFSLLHEQKRILDLQSKLIIAVDVAKGMEEYLHNLT QPIIHRDLNSHNILLYEDGHAVVADFGESRFLQSLDEDNMTKQPGNLRWMAPEVFTQCTRYTI KADVFSYALCLWEILTGEIPFABLKPAAAAADMAYHEERPPIGYSIPKPISSLLIRGWNACPE corresponding to amino acids 14-707 of NP_{—}057062 (SEQ. ID NO:210), which also corresponds to amino acids 115-808 of C03950_{—}3_P31 (SEQ. ID NO:228), and a third amino acid sequence being at least 70%, optionally at least 80%, preferably at least 85%, more preferably at least 90% and most preferably at least 95% homologous to a polypeptide having the sequence AKSRPSHYPVSSVYTETLKKKNEDRFGMWIEYLRR (SEQ. ID NO: 356) corresponding to amino acids 809-843 of C03950_{—}3_P31 (SEQ. ID NO:228), wherein said first amino acid sequence, second amino acid sequence and third amino acid sequence are contiguous and in a sequential order.

B. An isolated polypeptide encoding for a head of C03950_{—}3_P31 (SEQ. ID NO:228), comprising a polypeptide being at least 70%, optionally at least about 80%, preferably at least about 85%, more preferably at least about 90% and most preferably at least about 95% homologous to the sequence MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHS (SEQ. ID NO: 379) of C03950_{—}3_P31 (SEQ. ID NO:228).

C. An isolated polypeptide encoding for an edge portion of C03950_{—}3 P31 (SEQ. ID NO:228), comprising an amino acid sequence being at least 70%, optionally at least about 80%, preferably at least about 85%, more preferably at least about 90% and most preferably at least about 95% homologous to the sequence AKSRPSHYPVSSVYTETLKKKNEDRFGMWIEYLRR (SEQ. ID NO: 356) of C03950_{—}3_P31 (SEQ. ID NO:228).

The localization of the variant protein was determined according to results from a number of different software programs and analyses, including analyses from SignalP and other specialized programs. The variant protein is believed to be located intracellularly.

Variant protein C03950_{—}3_P31 (SEQ. ID NO:228) also has the following non-silent SNPs (Single Nucleotide Polymorphisms) as listed in Table 116, (given according to their position(s) on the amino acid sequence, with the alternative amino acid(s) listed.

TABLE 116
Amino acid mutations

| SNP position(s) on amino acid | |
|---|---|
| sequence | Alternative amino acid(s) |
| 108 | T -> P |

The variant protein has the following domains, as determined by using InterPro. The domains are described in Table 117:

TABLE 117
InterPro domain(s)

| Domain | | |
|---|---|---|
| description | Analysis type | Position(s) on protein |
| Protein kinase | BlastProDom | 564-766 |
| Ankyrin | FPrintScan | 268-280, 453-465 |
| Ankyrin | HMMPfam | 167-200, 201-233, 234-266, 267-299, |
| | | 300-334, 335-365, 370-403, 405-439, |
| | | 440-472, 482-514 |
| Protein kinase | HMMPfam | 564-821 |
| Tyrosine protein | HMMSmart | 564-822 |
| kinase | | |
| Serine | HMMSmart | 564-823 |
| Ankyrin | HMMSmart | 167-197, 201-230, 234-263, 267-296, |
| | | 300-331, 335-366, 370-401, 405-436, |
| | | 440-469, 482-511 |
| Protein kinase | ProfileScan | 564-826 |
| Ankyrin | ProfileScan | 201-233, 234-266, 270-299, 300-332, |
| | | 370-402, 440-472 |
| Ankyrin | ProfileScan | 159-502 |
| Protein kinase | ScanRegExp | 570-591 |

Variant protein C03950_{—}3_P31 (SEQ. ID NO:228) is encoded by the following transcript(s): C03950_{—}3_T10 (SEQ. ID NO:161), for which the coding portion starts at position 36 and ends at position 2564. The transcript also has the following SNPs as listed in Table 118 (given according to their position on the nucleotide sequence, with the alternative nucleic acid listed.

TABLE 118
Nucleic acid SNPs

| SNP position(s) on nucleotide | |
|---|---|
| sequence | Alternative nucleic acid(s) |
| 263 | A -> G |
| 357 | A -> C |
| 2677 | T -> C |

Variant protein C03950_{—}3P33 (SEQ. ID NO:229) according to the present invention is encoded by transcript C03950 3_T13 (SEQ. ID NO:163). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

1. Comparison report between C03950_{—}3_P33 (SEQ. ID NO:229) and TNI3K_HUMAN (SEQ. ID NO: 396):

A. An isolated chimeric polypeptide encoding for C03950_{—}3_P33 (SEQ. ID NO:229), comprising a first amino acid sequence being at least 90% homologous to MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHSDEWKKKVSES YVITIERLEDDLQIKEKELTELRNIFGSDEAFSKVNLNYRTENGLSLLHLCCICGGKKSHIRTLM LKGLRPSRLTRNGFTALHLAVYKDNAELITSLLHSGADIQQVGYGGLTALHIATIAGHLEAAD VLLQHGANVNIQDAVFFTPLHIAAYYGHEQVTRLLLKFGADVNVSGEVGDRPLHLASAKGFL NIAKLLMEEGSKADVNAQDNEDHVPLHFCSRFGHHDIVKYLLQSDLEVQPHVVNIYGDTPLH LACYNGKFEVAKEIIQISGTESLTKENIFSETAFHSACTYGKSIDLVKFLLDQNVININHQGRDG HTGLHSACYHGHIRLVQFLLDNGADMNLVACDPSRSSGEKDEQTCLMWAYEKGHDAIVTLL KHYKRPQDELPCNEYSQPGGDGSYVSVPSPLGKIKSMTKEKADILLLRAGLPSHFHLQLSEIEF HEIIGSGSFGKVYKGRCRNKIVAIKRYRANTYCSKSDVDMFCREVSILCQLNHPCVIQFVGAC LNDPSQFAIVTQYISGGSLFSLLHEQKRILDLQSKLIIAVDVAKGMEYLHNLTQPIIHRDLN corresponding to amino acids 1-691 of TNI3K_HUMAN (SEQ. ID NO: 396), which also corresponds to amino acids 1-691 of C03950_{—}3_P33 (SEQ. ID NO:229), and a second amino acid sequence being at least 70%, optionally at least 80%, preferably at least 85%, more preferably at least 90% and most preferably at least 95% homologous to a polypeptide having the sequence RYFFPK (SEQ. ID NO: 364) corresponding to amino acids 692-697 of C03950_{—}3_P33 (SEQ. ID NO:229), wherein said first amino acid sequence and second amino acid sequence are contiguous and in a sequential order.

B. An isolated polypeptide encoding for an edge portion of C03950_{—}3_P33 (SEQ. ID NO:229), comprising an amino acid sequence being at least 70%, optionally at least about 80%, preferably at least about 85%, more preferably at least about 90% and most preferably at least about 95% homologous to the sequence RYFFPK (SEQ. ID NO: 364) of C03950_{—}3_P33 (SEQ. ID NO:229).

2. Comparison report between C03950_{—}3_P33 (SEQ. ID NO:229) and Q6MZS9_HUMAN (SEQ. ID NO:211):

A. An isolated chimeric polypeptide encoding for C03950_{—}3_P33 (SEQ. ID NO:229), comprising a first amino acid sequence being at least 90% homologous to MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHSDEWKKKVSES YVITIERLEDDLQIKEKELTELRNIFGSDEAFSKVNLNYRTENGLSLLHLCCICGGKKSHERTLM LKGLRPSRLTRNGFTALHLAVYKDNAELITSLLHSGADIQQVGYGGLTALHIATIAGHLEAAD VLLQHGANVNIQDAVFFTPLHIAAYYGHEQVTRLLLKFGADVNVSGEVGDRPLHLASAKGFL NIAKLLMEEGSKADVNAQDNEDHVPLHFCSRFGHHD corresponding to amino acids 18-367 of Q6MZS9_HUMAN (SEQ. ID NO:211), which also corresponds to amino acids 1-350 of C03950_{—}3_P33 (SEQ. ID NO:229), a bridging amino acid I corresponding to amino acid 351 of C03950_{—}3_P33 (SEQ. ID NO:229), a second amino acid sequence being at least 90% homologous to VKYLLQSDLEVQPHVVNIYGDTPLHLACYNGKFEVAKEIIQISGTESLTKENIFSETAFHSACT YGKSIDLVKFLLDQNVININHQGRDGHTGLHSACYHGBIRLVQFLLDNGADM corresponding to amino acids 369-484 of Q6MZS9_HUMAN (SEQ. ID NO:211), which also corresponds to amino acids 352-467 of C03950_{—}3_P33 (SEQ. ID NO:229), a bridging amino acid N corresponding to amino acid 468 of C03950_{—}3_P33 (SEQ. ID NO:229), and a third amino acid sequence being at least 90% homologous to LVACDPSRSSGEKDEQTCLMWAYEKGHDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNHPCVIQFVGACLNDPSQFAIVTQYISGGSLFSLLHEQKRI LDLQSKLIIAVDVAKGMEYLHNLTQPIIHRDLNRYFFPK corresponding to amino acids 486-714 of Q6MZS9_HUMAN (SEQ. ID NO:211), which also corresponds to amino acids 469-697 of C03950_{—}3_P33 (SEQ. ID NO:229), wherein said, first amino acid sequence, bridging amino acid, second amino acid sequence, bridging amino acid and third amino acid sequence are contiguous and in a sequential order.

3. Comparison report between C03950_{—}3_P33 (SEQ. ID NO:229) and NP_{—}057062 (SEQ. ID NO:210):

A. An isolated chimeric polypeptide encoding for C03950_{—}3_P33 (SEQ. ID NO:229), comprising a first amino acid sequence being at least 70%, optionally at least 80%, preferably at least 85%, more preferably at least 90% and most preferably at least 95%, homologous to a polypeptide having the sequence MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHS (SEQ. ID NO: 379) corresponding to amino acids 1-114 of C03950_{—}3_P33 (SEQ. ID NO:229), a second amino acid sequence being at least 90% homologous to DEWKKKVSESYVITIERLEDDLQIKEKELTELRNIFGSDEAFSKVNLNYRTENGLSLLHLCCIC GGKKSHIRTLMLKGLRPSRLTRNGFTALHLAVYKDNAELITSLLHSGADIQQVGYGGLTALHI ATIAGBLEAADVLLQHGANVNIQDAVFFTPLHIAAYYGHEQVTRILLKFGADVNVSGEVGDR PLHLASAKGFLNIAKLLMEEGSKADVNAQDNEDHVPLHFCSRFGHHDIVKYLLQSDLEVQPH VVNIYGDTPLHLACYNGKFEVAKIIQISGTESLTKENIFSETAFHSACTYGKSIDLKFLLDQN VININHQGRDGHTGLHSACYHGHIRLVQFLLDNGADMNLVACDPSRSSGEKDEQTCLMWAY EKGHDAIVTLLKHYKRPQDELPCNEYSQPGGDGSYVSVPSPLGKIKSMTKEKADILLLRAGLP SHFHLQLSEIEFHEIIGSGSFGKVYKGRCRNKIVAIKRYRANTYCSKSDVDMFCREVSILCQLN HPCVIQFVGACLNDPSQFAIVTQYISGGSLFSLLHEQKRILDLQSKLIIAVDVAKGMEYLHNLT QPIIHRDLN corresponding to amino acids 14-590 of NP_{—}057062 (SEQ. ID NO:210), which also corresponds to amino acids 115-691 of C03950_{—}3_P33 (SEQ. ID NO:229), and a third amino acid sequence being at least 70%, optionally at least 80%, preferably at least 85%, more preferably at least 90% and most preferably at least 95% homologous to a polypeptide having the sequence RYFFPK (SEQ. ID NO: 364) corresponding to amino acids 692-697 of C03950_{—}3_P33 (SEQ. ID NO:229), wherein said first amino acid sequence, second amino acid sequence and third amino acid sequence are contiguous and in a sequential order.

B. An isolated polypeptide encoding for a head of C03950_{—}3_P33 (SEQ. ID NO:229), comprising a polypeptide being at least 70%, optionally at least about 80%, preferably at least about 85%, more preferably at least about 90% and most preferably at least about 95% homologous to the sequence MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHS (SEQ. ID NO: 379) of C03950_{—}3_P33 (SEQ. ID NO:229).

C. An isolated polypeptide encoding for an edge portion of C03950_{—}3_P33 (SEQ. ID NO:229), comprising an amino acid sequence being at least 70%, optionally at least about 80%, preferably at least about 85%, more preferably at least about 90% and most preferably at least about 95% homologous to the sequence RYFFPK (SEQ. ID NO: 364) of C039503_P33 (SEQ. ID NO:229).

The localization of the variant protein was determined according to results from a number of different software programs and analyses, including analyses from SignalP and other specialized programs. The variant protein is believed to be located intracellularly.

Variant protein C03950 3_P33 (SEQ. ID NO:229) also has the following non-silent SNPs (Single Nucleotide Polymorphisms) as listed in Table 119, (given according to their position(s) on the amino acid sequence, with the alternative amino acid(s) listed.

TABLE 119
Amino acid mutations

| SNP position(s) on amino acid | |
|---|---|
| sequence | Alternative amino acid(s) |
| 108 | T -> P |

The variant protein has the following domains, as determined by using InterPro. The domains are described in Table 120:

TABLE 120
InterPro domain(s)

| Domain | | |
|---|---|---|
| description | Analysis type | Position(s) on protein |
| Protein kinase | BlastProDom | 564-693 |
| Ankyrin | FPrintScan | 268-280, 453-465 |
| Ankyrin | HMMPfam | 167-200, 201-233, 234-266, 267-299, |
| | | 300-334, 335-365, 370-403, 405-439, |
| | | 440-472, 482-514 |
| Tyrosine protein | HMMSmart | 564-697 |
| kinase | | |
| Serine | HMMSmart | 564-694 |
| Ankyrin | HMMSmart | 167-197, 201-230, 234-263, 267-296, |
| | | 300-331, 335-366, 370-401, 405-436, |
| | | 440-469, 482-511 |
| Protein kinase | ProfileScan | 564-697 |
| Ankyrin | ProfileScan | 201-233, 234-266, 270-299, 300-332, |
| | | 370-402, 440-472 |
| Ankyrin | ProfileScan | 159-502 |
| Protein kinase | ScanRegExp | 570-591 |

Variant protein C03950_{—}3_P33 (SEQ. ID NO:229) is encoded by the following transcript(s): C03950_{—}3_T13 (SEQ. ID NO:163), for which the coding portion starts at position 36 and ends at position 2126. The transcript also has the following SNPs as listed in Table 121 (given according to their position on the nucleotide sequence, with the alternative nucleic acid listed.

TABLE 121
Nucleic acid SNPs

| SNP position(s) on nucleotide | |
|---|---|
| sequence | Alternative nucleic acid(s) |
| 263 | A -> G |
| 357 | A -> C |
| 7207 | C -> A |

Variant protein C03950_{—}3_P35 (SEQ. ID NO:230) according to the present invention is encoded by transcript C03950_{—}3_{—l T}18 (SEQ. ID NO:166). One or more alignments to one or more previously published Serine/threonine-protein kinase TNNI3K (SEQ. ID NO:209) protein sequences are given in the alignment table on the attached CD-ROM. A brief description of the relationship of the variant protein according to the present invention to each such aligned protein is as follows:

1. Comparison report between C03950_{—}3_P35 (SEQ. ID NO:230) and TNI3K HUMAN (SEQ. ID NO: 396):

A. An isolated chimeric polypeptide encoding for C03950_{—}3_P35 (SEQ. ID NO:230), comprising a first amino acid sequence being at least 90% homologous to MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLHSDEWKKKVSES YVITIERLEDDLQIKEKELTELRNIFGSDEAFSKVNLNYRTENGLSLLHLCCICGGKKSHERTLM LKGLRPSRLTRNGFTALHLAVYKDNAELITSLLHSGADIQQVGYGGLTALHIATIAGHLEAAD VLLQHGANVNIQDAVFFTPLHIAAYYGHEQVTRLLLKFGADVNVSGEVGDRPLHLASAKGFL NIAKLLMEEGSKADVNAQDNEDHVPLHFCSRFGHHDIVKYLLQSDLEVQPHVVNIYGDTPLH LACYNGKFEVAKEIIQISGTESLTKENIFSETAPHSACTYGKSIDLVKFLLDQNVININHQGRDG HTGLHSACYHGHIRLVQFLLDNGADMNLVACDPSRSSGEKDEQTCLMWAYEKGHDAIVTLL KHYKRPQDELPCNEYSQPGGDGSYVSVPSPLGKIKSMTKEKADILLLRAGLPSHFHLQLSEIEF HEIIGSGSFGKVYKGRCRNKIVAIKRYRANTYCSKSDVDMFCREVSILCQLNHPCVIQFVGAC LNDPSQFAIVTQYISGGSLFSLLHEQKR corresponding to amino acids 1-657 of TNI3K_HUMAN (SEQ. ID NO: 396), which also corresponds to amino acids 1-657 of C03950_{—}3_P35 (SEQ. ID NO:230), and a second amino acid sequence being at least 70%, optionally at least 80%, preferably at least 85%, more preferably at least 90% and most preferbly at least 95% homologous to a polypeptide having the sequence YGSFVLIYPWTFRRNYSCNTSEGFPLDEPSPFEI (SEQ. ID NO: 372) corresponding to amino acids 658-691 of C03950_{—}3_P35 (SEQ. ID NO:230), wherein said first amino acid sequence and second amino acid sequence are contiguous and in a sequential order.

B. An isolated polypeptide encoding for an edge portion of C03950_{—}3_P35 (SEQ. ID NO:230), comprising an amino acid sequence being at least 70%, optionally at least about 80%, preferably at least about 85%, more preferably at least about 90% and most preferably at least about 95% homologous to the sequence YGSFVLIYPWTFRRNYSCNTSEGFPLDEPSPFEI (SEQ. ID NO: 372) of C03950_{—}3_P35 (SEQ. ID NO:230).

2. Comparison report between C03950_{—}3_P35 (SEQ. ID NO:230) and Q6MZS9_HUMAN (SEQ. ID NO:211):

A. An isolated chimeric polypeptide encoding for C03950_{—}3_P35 (SEQ. ID NO:230), comprising a first amino acid sequence being at least 90% homologous to MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHSDEWKKKVSES YVITLERLEDDLQIKEKELTELRNIFGSDEAFSKVNLNYRTENGLSLLHLCCICGGKKSHIRTLM LKGLRPSRLTRNGFTALHLAVYKDNAELITSLLHSGADIQQVGYGGLTALHIATIAGHLEAAD VLLQHGANVNIQDAVFFTPLHIAAYYGHEQVTRLLLKFGADVNVSGEVGDRPLHLASAKGFL NIAKLLMEEGSKADVNAQDNEDHVPLHFCSRFGHED corresponding to amino acids 18-367 of Q6MZS9_HUMAN (SEQ. ID NO:211), which also corresponds to amino acids 1-350 of C03950_{—}3_P35 (SEQ. ID NO:230), a bridging amino acid I corresponding to amino acid 351 of C03950_{—}3_P35 (SEQ. ID NO:230), a second amino acid sequence being at least 90% homologous to VKYLLQSDLEVQPHVVNIYGDTPLIMACYNGKFEVAKEIIQISGTESLTKENIFSETAFHSACT YGKSIDLVKFLLDQNVININHQGRDGHTGLHSACYHGHIRLVQFLLDNGADM corresponding to amino acids 369-484 of Q6MZS9_HUMAN (SEQ. ID NO:211), which also corresponds to amino acids 352-467 of C03950_{—}3_P35 (SEQ. ID NO:230), a bridging amino acid N corresponding to amino acid 468 of C03950_{—}3_P35 (SEQ. ID NO:230), a third amino acid sequence being at least 90% homologous to LVACDPSRSSGEKDEQTCLMWAYEKGHDAIVTLLKHYKRPQDELPCNEYSQPGGDGSYVSV PSPLGKIKSMTKEKADILLLRAGLPSHFHLQLSEIEFHEIIGSGSFGKVYKGRCRNKIVAIKRYR ANTYCSKSDVDMFCREVSILCQLNHPCVIQFVGACLNDPSQFAIVTQYISGGSLFSLLHEQKR corresponding to amino acids 486-674 of Q6MZS9_HUMAN (SEQ. ID NO:211), which also corresponds to amino acids 469-657 of C03950_{—}3_P35 (SEQ. ID NO:230), and a fourth amino acid sequence being at least 70%, optionally at least 80%, preferably at least 85%, more preferably at least 90% and most preferably at least 95% homologous to a polypeptide having the sequence YGSFVLIYPWTFRRNYSCNTSEGFPLDEPSPFEI (SEQ. ID NO: 372) corresponding to amino acids 658-691 of C03950_{—}3_P35 (SEQ. ID NO:230), wherein said , first amino acid sequence, bridging amino acid, second amino acid sequence, bridging amino acid, third amino acid sequence and fourth amino acid sequence are contiguous and in a sequential order.

B. An isolated polypeptide encoding for an edge portion of C03950_{—}3_P35 (SEQ. ID NO:230), comprising an amino acid sequence being at least 70%, optionally at least about 80%, preferably at least about 85%, more preferably at least about 90% and most preferably at least about 95% homologous to the sequence YGSFVLIYPWTFRRNYSCNTSEGFPLDEPSPFEI (SEQ. ID NO: 372) of C03950_{—}3_P35 (SEQ. ID NO:230).

3. Comparison report between C03950_{—}3_P35 (SEQ. ID NO:230) and NP_{—}057062 (SEQ. ID NO:210):

A. An isolated chimeric polypeptide encoding for C03950_{—}3_P35 (SEQ. ID NO:230), comprising a first amino acid sequence being at least 70%, optionally at least 80%, preferably at least. 85%, more preferably at least 90% and most preferably at least 95%, homologous to a polypeptide having the sequence MAAARDPFEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHS (SEQ. ID NO: 379) corresponding to amino acids 1-114 of C03950_{—}3_P35 (SEQ. ID NO:230), a second amino acid sequence being at least 90% homologous to DEWKKKVSESYVITIERLEDDLQIKEKELTELRNIFGSDEAFSKVNLNYRTENGLSLLHLCCIC GGKKSHIRTLMLKGLRPSRLTRNGFTALHLAVYKDNAELITSLLHSGADIQQVGYGGLTALHI ATIAGHLEAADVLLQHGANVNIQDAVFFTPLHIAAYYGHEQVTRLLLKFGADVNVSGEVGDR PLHLASAKGFLNIAKLLMEEGSKADVNAQDNEDHVPLHFCSRFGHHDIVKYLLQSDLEVQPH VVNIYGDTPLHLACYNGKFEVAKEIIQISGTESLTKENIFSETAFHSACTYGKSIDLVKFLLDQN VININHQGRDGHTGLHSACYHGHIRLVQFLLDNGADMNLVACDPSRSSGEKDEQTCLMWAY EKGHDAIVTLLKHYKRPQDELPCNEYSQPGGDGSYVSVPSPLGKIKSMTKEKADILLLRAGLP SHFHIQLSELEFHEIIGSGSFGKVYKGRCRNKIVAIKRYRANTYCSKSDVDMFCREVSILCQLN HPCVIQFVGACLNDPSQFAIVTQYISGGSLFSLLHEQKR corresponding to amino acids 14-556 of NP_{—}057062 (SEQ. ID NO:210), which also corresponds to amino acids 115-657 of C03950_{—}3235 (SEQ. ID NO:230), and a third amino acid sequence being at least 70%, optionally at least 80%, preferably at least 85%, more preferably at least 90% and most preferably at least 95% homologous to a polypeptide having the sequence YGSFVLIYPWTFRRNYSCNTSEGFPLDEPSPFEI (SEQ. ID NO: 372) corresponding to amino acids 658-691 of C03950_{—}3_P35 (SEQ. ID NO:230), wherein said first amino acid sequence, second amino acid sequence and third amino acid sequence are contiguous and in a sequential order.

B. An isolated polypeptide encoding for a head of C03950_{—}3_P35 (SEQ. ID NO:230), comprising a polypeptide being at least 70%, optionally at least about 80%, preferably at least about 85%, more preferably at least about 90% and most preferably at least about 95% homologous to the sequence MAAARDPPEVSLREATQRKLRRFSELRGKLVARGEFWDIVAITAADEKQELAYNQQLSEKLK RKELPLGVQYHVFVDPAGAKIGNGGSTLCALQCLEKLYGDKWNSFTILLIHS (SEQ. ID NO: 379) of C03950_{—}3_P35 (SEQ. ID NO:230).

The localization of the variant protein was determined according to results from a number of different software programs and analyses, including analyses from SignalP and other specialized programs. The variant protein is believed to be located intracellularly.

Variant protein C03950_{—}3_P35 (SEQ. ID NO:230) also has the following non-silent SNPs (Single Nucleotide Polymorphisms) as listed in Table 122, (given according to their position(s) on the amino acid sequence, with the alternative amino acid(s) listed.

TABLE 122
Amino acid mutations

| SNP position(s) on amino acid | |
|---|---|
| sequence | Alternative amino acid(s) |
| 108 | T -> P |

The variant protein has the following domains, as determined by using InterPro. The domains are described in Table 123:

TABLE 123
InterPro domain(s)

| Domain | | |
|---|---|---|
| description | Analysis type | Position(s) on protein |
| Protein kinase | BlastProDom | 564-657 |
| Ankyrin | FPrintScan | 268-280, 453-465 |
| Ankyrin | HMMPfam | 167-200, 201-233, 234-266, 267-299, |
| | | 300-334, 335-365, 370-403, 405-439, |
| | | 440-472, 482-514 |
| Tyrosine protein | HMMSmart | 564-691 |
| kinase | | |
| Serine | HMMSmart | 564-689 |
| Ankyrin | HMMSmart | 167-197, 201-230, 234-263, 267-296, |
| | | 300-331, 335-366, 370-401, 405-436, |
| | | 440-469, 482-511 |
| Protein kinase | ProfileScan | 564-691 |
| Ankyrin | ProfileScan | 201-233, 234-266, 270-299, 300-332, |
| | | 370-402, 440-472 |
| Ankyrin | ProfileScan | 159-502 |
| Protein kinase | ScanRegExp | 570-591 |

Variant protein C03950_{—}3 P35 (SEQ. ID NO:230) is encoded by the following transcript(s): C03950_{—}3_T18 (SEQ. ID NO:166), for which the coding portion starts at position 36 and ends at position 2108. The transcript also has the following SNPs as listed in Table 124 (given according to their position on the nucleotide sequence, with the alternative nucleic acid listed.

TABLE 124
Nucleic acid SNPs

| SNP position(s) on nucleotide | |
|---|---|
| sequence | Alternative nucleic acid(s) |
| 263 | A -> G |
| 357 | A -> C |

As noted above, cluster C03950 features 38 segment(s), which were listed in Table 86 above and for which the sequence(s) are given. These segment(s) are portions of nucleic acid sequence(s) which are described herein separately because they are of particular interest. A description of several segments according to the present invention is now provided.

Segment cluster C03950_{—}3_N44 (SEQ. ID NO:176) according to the present invention is supported by 21 libraries. The number of libraries was determined as previously described. This segment can be found in the following transcript(s): C03950_{—}3 T10 (SEQ. ID NO:161), C03950_{—}3_T11 (SEQ. ID NO:162), C03950_{—}3_T13 (SEQ. ID NO:163), C03950_{—}3_T15 (SEQ. ID NO:164), C03950_{—}3_T17 (SEQ. ID NO:165), C03950_{—}3_T18 (SEQ. ID NO:166), C03950_{—}3_{—T}19 (SEQ. ID NO:167), C03950_{—}3_T2 (SEQ. ID NO:156), C03950_{—}3 T4 (SEQ. ID NO:157), C03950_{—}3_T7 (SEQ. ID NO:158), C03950_{—}3_T8 (SEQ. ID NO:159) and C03950_{—}3_T9 (SEQ. ID NO:160). Table 125 below describes the starting and ending position of this segment on each transcript.

TABLE 125
Segment location on transcripts

| | Segment | Segment |
|---|---|---|
| Transcript name | starting position | ending position |
| C03950_3_T10 (SEQ. ID NO: 161) | 1811 | 2005 |
| C03950_3_T11 (SEQ. ID NO: 162) | 1861 | 2055 |
| C03950_3_T13 (SEQ. ID NO: 163) | 1811 | 2005 |
| C03950_3_T15 (SEQ. ID NO: 164) | 1861 | 2055 |
| C03950_3_T17 (SEQ. ID NO: 165) | 1861 | 2055 |
| C03950_3_T18 (SEQ. ID NO: 166) | 1811 | 2005 |
| C03950_3_T19 (SEQ. ID NO: 167) | 1861 | 2055 |
| C03950_3_T2 (SEQ. ID NO: 156) | 1811 | 2005 |
| C03950_3_T4 (SEQ. ID NO: 157) | 1861 | 2055 |
| C03950_3_T7 (SEQ. ID NO: 158) | 1861 | 2055 |
| C03950_3_T8 (SEQ. ID NO: 159) | 1185 | 1379 |
| C03950_3_T9 (SEQ. ID NO: 160) | 1185 | 1379 |

Segment cluster C03950_{—}3 N45 (SEQ. ID NO:177), according to the present invention is supported by 2 libraries. The number of libraries was determined as previously described. This segment can be found in the following transcript(s): C03950_{—}3_T18 (SEQ. ID NO:166) and C03950_{—}3_T19 (SEQ. ID NO:167). Table 126 below describes the starting and ending position of this segment on each transcript.

TABLE 126
Segment location on transcripts

| | Segment | Segment |
|---|---|---|
| Transcript name | starting position | ending position |
| C03950_3_T18 (SEQ. ID NO: 166) | 2006 | 2339 |
| C03950_3_T19 (SEQ. ID NO: 167) | 2056 | 2389 |

Segment cluster C03950_{—}3 N48 (SEQ. ID NO:178) according to the present invention is supported by 2 libraries. The number of libraries was determined as previously described. This segment can be found in the following transcript(s): C03950_{—}3 T13 (SEQ. ID NO:163). Table 127 below describes the starting and ending position of this segment on each transcript.

TABLE 127
Segment location on transcripts

| | Segment | Segment |
|---|---|---|
| Transcript name | starting position | ending position |
| C03950_3_T13 (SEQ. ID NO: 163) | 2111 | 7282 |

Segment cluster C03950_{—}3 N49 (SEQ. ID NO:179) according to the present invention is supported by 10 libraries. The number of libraries was determined as previously described. This segment can be found in the following transcript(s): C03950_{—}3_T13 (SEQ. ID NO:163) and C03950_{—}3.315 (SEQ. ID NO:164). Table 128 below describes the starting and ending position of this segment on each transcript.

TABLE 128
Segment location on transcripts

| | Segment | Segment |
|---|---|---|
| Transcript name | starting position | ending position |
| C03950_3_T13 (SEQ. ID NO: 163) | 7283 | 7566 |
| C03950_3_T15 (SEQ. ID NO: 164) | 2161 | 2444 |

Segment cluster C03950_{—}3_N56 (SEQ. ID NO:180) according to the present invention is supported by 8 libraries. The number of libraries was determined as previously described. This segment can be found in the following transcript(s): C03950_{—}3_T17 (SEQ. ID NO:165). Table 129 below describes the starting and ending position of this segment on each transcript.

TABLE 129
Segment location on transcripts

| | Segment | Segment |
|---|---|---|
| Transcript name | starting position | ending position |
| C03950_3_T17 (SEQ. ID NO: 165) | 2161 | 2352 |

Segment cluster C03950_{—}3 N62 (SEQ. ID NO:181) according to the present invention is supported by 2 libraries. The number of libraries was determined as previously described. This segment can be found in the following transcript(s): C03950_{—}3_T22 (SEQ. ID NO:169) and C03950_{—}3_T23 (SEQ. ID NO:170). Table 130 below describes the starting and ending position of this segment on each transcript.

TABLE 130
Segment location on transcripts

| | Segment | Segment |
|---|---|---|
| Transcript name | starting position | ending position |
| C03950_3_T22 (SEQ. ID NO: 169) | 1 | 591 |
| C03950_3_T23 (SEQ. ID NO: 170) | 1 | 591 |

Segment cluster C039503 N67 (SEQ. ID NO:183) according to the present invention is supported by 3 libraries. The number of libraries was determined as previously described. This segment can be found in the following transcript(s): C03950_{—}3_T10 (SEQ. ID NO:161) and C03950_{—}3_T11 (SEQ. ID NO:162). Table 131 below describes the starting and ending position of this segment on each transcript.

TABLE 131
Segment location on transcripts

| | Segment | Segment |
|---|---|---|
| Transcript name | starting position | ending position |
| C03950_3_T10 (SEQ. ID NO: 161) | 2460 | 2750 |
| C03950_3_T11 (SEQ. ID NO: 162) | 2510 | 2800 |

According to an optional embodiment of the present invention, short segments related to the above cluster are also provided. These segments are up to about 120 by in length, and so are included in a separate description.

Segment cluster C03950_{—}3_N17 (SEQ. ID NO:191) according to the present invention is supported by 1 libraries. The number of libraries was determined as previously described. This segment can be found in the following transcript(s): C03950_{—}3_T8 (SEQ. ID NO:159) and C03950_{—}3_T9 (SEQ. ID NO:160). Table 132 below describes the starting and ending position of this segment on each transcript.

TABLE 132
Segment location on transcripts

| | Segment | Segment |
|---|---|---|
| Transcript name | starting position | ending position |
| C03950_3_T8 (SEQ. ID NO: 159) | 1 | 45 |
| C03950_3_T9 (SEQ. ID NO: 160) | 1 | 45 |

Segment cluster C03950_{—}3 N40 (SEQ. ID NO:199) according to the present invention is supported by 2 libraries. The number of libraries was determined as previously described. This segment can be found in the following transcript(s): C03950_{—}3_T21 (SEQ. ID NO:168). Table 133 below describes the starting and ending position of this segment on each transcript.

TABLE 133
Segment location on transcripts

| | Segment | Segment |
|---|---|---|
| Transcript name | starting position | ending position |
| C03950_3_T21 (SEQ. ID NO: 168) | 1803 | 1829 |

Segment cluster C039503 N51 (SEQ. ID NO:202) according to the present invention is supported by 1 libraries. The number of libraries was determined as previously described. This segment can be found in the following transcript(s): C03950_{—}3_T2 (SEQ. ID NO:156), C03950_{—}3_T4 (SEQ. ID NO:157) and C03950_{—}3_T9 (SEQ. ID NO:160). Table 134 below describes the starting and ending position of this segment on each transcript.

TABLE 134
Segment location on transcripts

| | Segment | Segment |
|---|---|---|
| Transcript name | starting position | ending position |
| C03950_3_T2 (SEQ. ID NO: 156) | 2111 | 2229 |
| C03950_3_T4 (SEQ. ID NO: 157) | 2161 | 2279 |
| C03950_3_T9 (SEQ. ID NO: 160) | 1485 | 1603 |

Segment cluster C03950_{—}3 N75 (SEQ. ID NO:208) according to the present invention is supported by 1 libraries. The number of libraries was determined as previously described. This segment can be found in the following transcript(s): C03950_{—}3_T23 (SEQ. ID NO:170) and C03950_{—}3 T7 (SEQ. ID NO:158). Table 135 below describes the starting and ending position of this segment on each transcript.

TABLE 135
Segment location on transcripts

| | Segment | Segment |
|---|---|---|
| Transcript name | starting position | ending position |
| C03950_3_T23 (SEQ. ID NO: 170) | 1145 | 1218 |
| C03950_3_T7 (SEQ. ID NO: 158) | 2820 | 2893 |

Expression of Homo sapiens TNNI3 interacting kinase (TNNI3K) C03950 transcripts which are detectable by amplicon as depicted in sequence name C03950_seg44WT (SEQ. ID NO: 233) specifically in heart tissue

Expression of Homo sapiens TNNI3 interacting kinase (TNNI3K) transcripts detectable by or according to seg44WT-C03950_seg44WT (SEQ. ID NO: 233) amplicon and primers C03950_seg44WTF (SEQ. ID NO: 231) and C03950_seg44WTR (SEQ. ID NO: 232) was measured by real time PCR. In parallel the expression of four housekeeping genes-SDHA (GenBank Accession No. NM_{—}004168 (SEQ. ID NO: 33); amplicon-SDHA-amplicon (SEQ. ID NO:36)), Ubiquitin (GenBank Accession No. BC000449 (SEQ. ID NO: 29); amplicon-Ubiquitin-amplicon (SEQ. ID NO: 32)), RPL19 (GenBank Accession No. NM_{—}000981 (SEQ. ID NO: 21); RPL19 amplicon (SEQ. ID NO: 24)) and TATA box (GenBank Accession No. NM_{—}003194 (SEQ. ID NO: 25); TATA amplicon (SEQ. ID NO: 28)) was measured similarly. For each RT sample, the expression of the above amplicon was normalized to the geometric mean of the quantities of the housekeeping genes. The normalized quantity of each RT sample was then divided by the median of the quantities of the heart samples (sample numbers 44, 45 and 46, Table 1_{—}6 above), to obtain a value of relative expression for each sample relative to median of the heart samples.

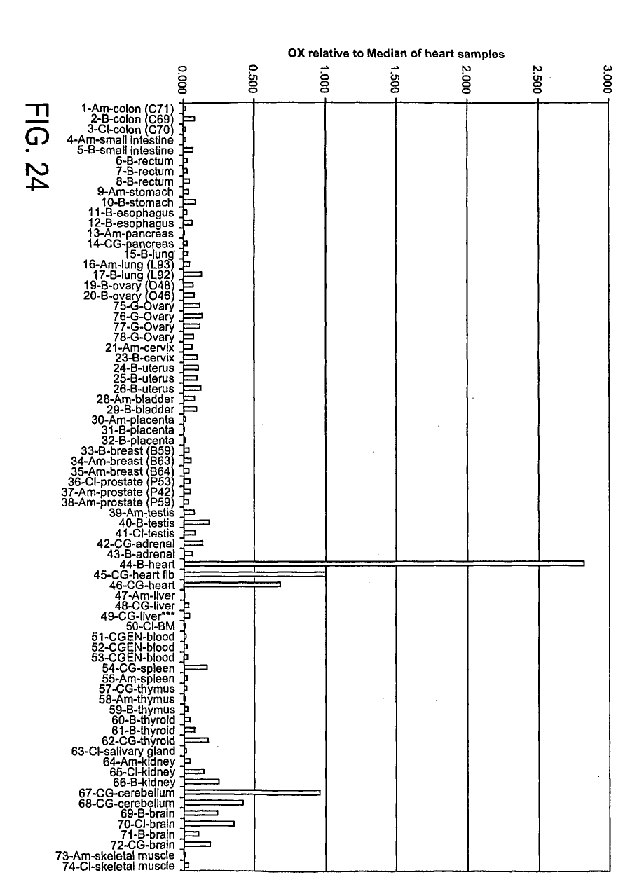

FIG. 24 is a histogram showing relative expression of the above₇indicated Homo sapiens TNNI3 interacting kinase (TNNI3K) transcripts in heart tissue samples as opposed to other tissues.

As is evident from FIG. 24, the expression of Homo sapiens TNNI3 interacting kinase (TNNI3K) transcripts detectable by the above amplicon in heart tissue samples was significantly higher than in most of the other samples (sample numbers 1, 2, 3, 4, 5, 6, 7, 8, 9, 10, 11, 12, 13, 14, 15, 16, 17, 19, 20, 75, 76, 77, 78, 21, 23, 24, 25, 26, 28, 29, 30, 31, 32, 33, 34, 35, 36, 37, 38, 39, 40, 41, 42, 43, 47, 48, 49, 50, 51, 52, 53, 54, 55, 57, 58, 59, 60, 61, 62, 63, 64, 65, 66, 67, 68, 69, 70, 71, 72, 73 and 74, Table 1_{—}6 above) except for the brain samples.

Primer pairs are also optionally and preferably encompassed within the present invention; for example, for the above experiment, the following primer pair was used as a non-limiting illustrative example only of a suitable primer pair: C03950_seg44WTF (SEQ. ID NO: 231) forward primer, and C03950_seg44WTR (SEQ. ID NO: 232) reverse primer.

The present invention also preferably encompasses any amplicon obtained through the use of any suitable primer pair; for example, for the above experiment, the following amplicon was obtained as a non-limiting illustrative example only of a suitable amplicon: C03950_seg44WT (SEQ. ID NO: 233).

Forward Primer C03950_seg44WTF
(SEQ. ID NO: 231): GAGCCAATACCTACTGCTCCAAG
Reverse Primer C03950_seg44WTR
(SEQ. ID NO: 232): GCAAGCACCCACAAACTGAATTA
Amplicon (C03950_seg44WT
(SEQ. ID NO: 233)): GAGCCAATACCTACTGCTCCAAGTCAGA
TGTGGATATGTTTTGCCGAGAGGTGTCCATTCTCTGCCAGCT
CAATCATCCCTGCGTAATTCAGTTTGTGGGTGCTTGC

Expression of Homo sapiens TNNI3 interacting kinase (TNNI3K) C03950 transcripts which are detectable by amplicon as depicted in sequence name C03950_seg51 (SEQ. ID NO: 236) specifically in heart tissue

Expression of Homo sapiens TNNI3 interacting kinase (TNNI3K) transcripts detectable by or according to seg51-C03950_seg51 (SEQ. ID NO: 236) amplicon and primers C03950_seg51F (SEQ. ID NO: 234) and C039500seg51R (SEQ. ID NO: 235) was measured by real time PCR. In parallel the expression of four housekeeping genes-SDHA (GenBank Accession No. NM_{—}004168 (SEQ. ID NO: 33); amplicon-SDHA-amplicon (SEQ. ID NO:36)), Ubiquitin (GenBank Accession No. BC000449 (SEQ. ID NO: 29); amplicon-Ubiquitin-amplicon (SEQ. ID NO: 32)), RPL19 (GenBank Accession No. NM_{—}000981 (SEQ. ID NO: 21); RPL19 amplicon (SEQ. ID NO: 24)) and TATA box (GenBank Accession No. NM_{—}003194 (SEQ. ID NO: 25); TATA amplicon (SEQ. ID NO: 28)) was measured similarly. For each RT sample, the expression of the above amplicon was normalized to the geometric mean of the quantities of the housekeeping genes. The normalized quantity of each RT sample was then divided by the median of the quantities of the heart samples (sample numbers 44, 45 and 46, Table 1_{—}6 above), to obtain a value of relative expression for each sample relative to median of the heart samples.

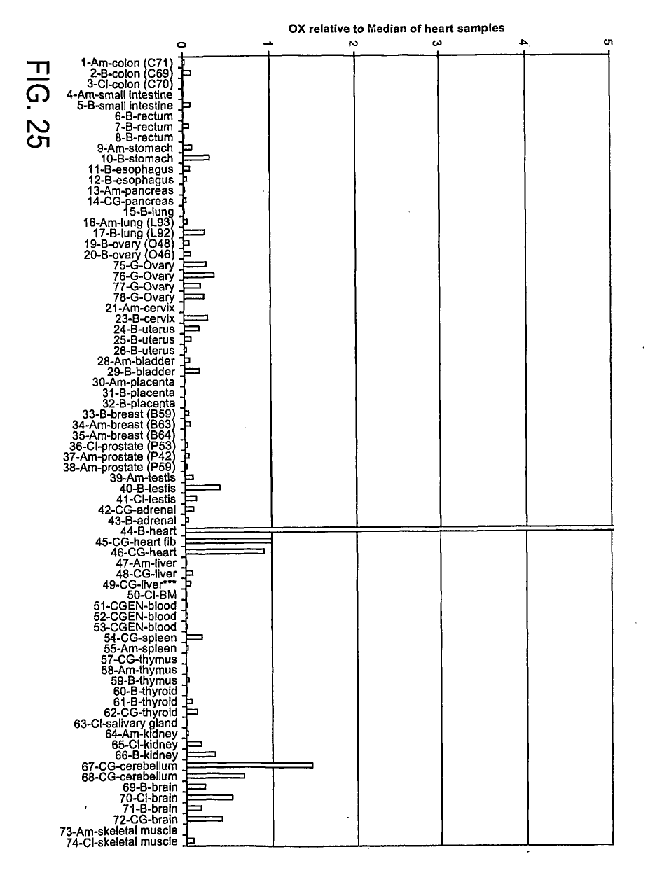

FIG. 25 is a histogram showing relative expression of the above-indicated Homo sapiens TNNI3 interacting kinase (TNNI3K) transcripts in heart tissue samples as opposed to other tissues.

As is evident from FIG. 25, the expression of Homo sapiens TNNI3 interacting kinase (TNNI3K) transcripts detectable by the above amplicon in heart tissue samples was significantly higher than in most of the other samples (sample numbers 1, 2, 3, 4, 5, 6, 7, 8, 9, 10, 11, 12, 13, 14, 15, 16, 17, 19, 20, 75, 76, 77, 78, 21, 23, 24, 25, 26, 28, 29, 30, 31, 32, 33, 34, 35, 36, 37, 38, 39, 40, 41, 42, 43, 47, 48, 49, 50, 51, 52, 53, 54, 55, 57, 58, 59, 60, 61, 62, 63, 64, 65, 66, 67, 68, 69, 70, 71, 72, 73 and 74, Table 1_{—}6 above) except for the brain samples.

Primer pairs are also optionally and preferably encompassed within the present invention; for example, for the above experiment, the following primer pair was used as a non-limiting illustrative example only of a suitable primer pair: C03950_seg51F (SEQ. ID NO: 234) forward primer; and C03950_seg51R (SEQ. ID NO: 235) reverse primer.

The present invention also preferably encompasses any amplicon obtained through the use of any suitable primer pair; for example, for the above experiment, the following amplicon was obtained as a non-limiting illustrative example only of a suitable amplicon: C03950_seg51 (SEQ. ID NO: 236).

Forward Primer (C03950_seg51F
(SEQ. ID NO: 234)): TCTGCCATTACCTCTAGGATCTGG
Reverse Primer (C03950_seg51R
(SEQ. ID NO: 235)): GGCAGAAGTAAGCATACACCTGAAA
Amplicon (C03950_seg51
(SEQ. ID NO: 236)): TCTGCCATTACCTCTAGGATCTGGATCA
CCCATAGTATTTGCATCTGGAGGGGAGCTCATTACTTTAACA
GGGAAGAATGCAATTTCAGGTGTATGCTTACTTCTGCC

Expression of Homo sapiens TNNI3 interacting kinase (TNNI3K) C03950 transcripts which are detectable by amplicon as depicted in sequence name C03950_seg67F2R2 (SEQ. ID NO: 239) specifically in heart tissue

Expression of Homo sapiens TNNI3 interacting kinase (TNNI3K) transcripts detectable by or according to seg67F2R2-C03950_seg67F2R2 (SEQ. ID NO: 239) amplicon and primers C03950_seg67F2 (SEQ. ID NO: 237) and C03950_seg67R2 (SEQ. ID NO: 238) was measure real time PCR. In parallel the expression of four housekeeping genes-SDHA (GenBank Accession No. NM_{—}004168 (SEQ. ID NO: 33); amplicon-SDHA-amplicon (SEQ. ID NO:36)), Ubiquitin (GenBank Accession No. BC000449 (SEQ. ID NO: 29); amplicon-Ubiquitin-amplicon (SEQ. ID NO: 32)), RPL19 (GenBank Accession No. NM_{—}000981 (SEQ. ID NO: 21); RPL19 amplicon (SEQ. ID NO: 24)) and TATA box (GenBank Accession No. NM_{—}003194 (SEQ. ID NO: 25); TATA amplicon (SEQ. ID NO: 28)) was measured similarly. For each RT sample, the expression of the above amplicon was normalized to the geometric mean of the quantities of the housekeeping genes. The normalized quantity of each RT sample was then divided by the median of the quantities of the heart samples (sample numbers 44, 45 and 46, Table 1_{—}6 above), to obtain a value of relative expression for each sample relative to median of the heart samples.

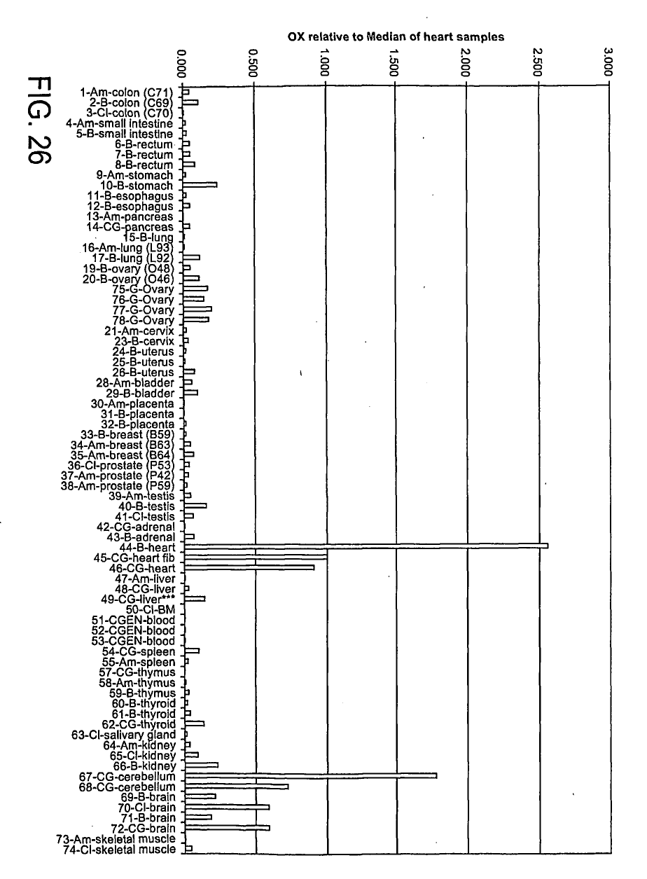

FIG. 26 is a histogram showing relative expression of the above-indicated Homo sapiens TNNI3 interacting kinase (TNNI3K) transcripts in heart tissue samples as opposed to other tissues.

As is evident from FIG. 26, the expression of Homo sapiens TNNI3 interacting kinase (TNNI3K) transcripts detectable by the above amplicon in heart tissue samples was significantly higher than in most of the other samples (sample numbers 1, 2, 3, 4, 5, 6, 7, 8, 9, 10, 11, 12, 13, 14, 15, 16, 17, 19, 20, 75, 76, 77, 78, 21, 23, 24, 25, 26, 28, 29, 30, 31, 32, 33, 34, 35, 36, 37, 38, 39, 40, 41, 42, 43, 47, 48, 49, 50, 51, 52, 53, 54, 55, 57, 58, 59, 60, 61, 62, 63, 64, 65, 66, 67, 68, 69, 70, 71, 72, 73 and 74, Table 1_{—}6 above) except for the brain samples.

Primer pairs are also optionally and preferably encompassed within the present invention; for example, for the above experiment, the following primer pair was used as a non-limiting illustrative example only of a suitable primer pair: C03950_seg67F2 (SEQ. ID NO: 237) forward primer; and C03950_seg67R2 (SEQ. ID NO: 238) reverse primer.

The present invention also preferably encompasses any amplicon obtained through the use of any suitable primer pair; for example, for the above experiment, the following amplicon was obtained as a non-limiting illustrative example only of a suitable amplicon: C03950_seg67F2R2 (SEQ. ID NO: 239).

Forward Primer C03950_seg67F2
(SEQ. ID NO: 237): CGTTTTGGGATGTGGATTGAGT
Reverse Primer C03950_seg67R2
(SEQ. ID NO: 238): ACCGCTTTCATGGAGCTAACA
Amplicon C03950_seg67F2R2
(SEQ. ID NO: 239) CGTTTTGGGATGTGGATTGAGTATCTCAGAAG
ATAACCTCTTATCCTGGCCATTCAACCTGATGTGTTACATGTTTATTTG
TTTAGAATCTTCCATCACTACCAAAATGTTAGCTCCATGAAAGCGGT

### Description for Cluster R15601

Cluster R15601 features 2 transcript(s) and 25 segment(s) of interest, the names for which are given in Tables 136 and 137, respectively. The selected protein variants are given in table 138.

TABLE 136
Transcripts of interest
Transcript Name

R15601_T8 (SEQ. ID NO: 240)
R15601_T9 (SEQ. ID NO: 241)

TABLE 137
Segments of interest
Segment Name

R15601_N4 (SEQ. ID NO: 242)
R15601_N6 (SEQ. ID NO: 243)
R15601_N10 (SEQ. ID NO: 244)
R15601_N14 (SEQ. ID NO: 245)
R15601_N16 (SEQ. ID NO: 246)
R15601_N18 (SEQ. ID NO: 247)
R15601_N20 (SEQ. ID NO: 248)
R15601_N22 (SEQ. ID NO: 249)
R15601_N26 (SEQ. ID NO: 250)
R15601_N28 (SEQ. ID NO: 251)
R15601_N30 (SEQ. ID NO: 252)
R15601_N32 (SEQ. ID NO: 253)
R15601_N42 (SEQ. ID NO: 254)
R15601_N45 (SEQ. ID NO: 255)
R15601_N0 (SEQ. ID NO: 256)
R15601_N3 (SEQ. ID NO: 257)
R15601_N8 (SEQ. ID NO: 258)
R15601_N12 (SEQ. ID NO: 259)
R15601_N24 (SEQ. ID NO: 260)
R15601_N27 (SEQ. ID NO: 261)
R15601_N34 (SEQ. ID NO: 262)
R15601_N36 (SEQ. ID NO: 263)
R15601_N38 (SEQ. ID NO: 264)
R15601_N40 (SEQ. ID NO: 265)
R15601_N44 (SEQ. ID NO: 266)

TABLE 138
Proteins of interest

| Protein Name | Corresponding Transcript(s) |
|---|---|
| R15601_P2 (SEQ. ID NO: 268) | R15601_T8 (SEQ. ID NO: 240) |
| R15601_P3 (SEQ. ID NO: 269) | R15601_T9 (SEQ. ID NO: 241) |

These sequences are variants of the known protein cardiomyopathy associated 4 (SEQ. ID NO:267) (SwissProt accession identifier NP 775259 (SEQ. ID NO: 397)), referred to herein as the previously known protein.

The sequence for protein cardiomyopathy associated 4 (SEQ. ID NO:267) is given.

Non-limiting exemplary utilities for R15601 variants according to the present invention are described in greater detail below and also with regard to the previous section on clinical utility. The heart-selective diagnostic marker prediction engine provided the following results with regard to cluster R15601. Predictions were made for selective expression of transcripts of this contig in heart tissue, according to the previously described methods. The numbers on the y-axis of the first figure below refer to weighted expression of ESTs in each category, as “parts per million” (ratio of the expression of ESTs for a particular cluster to the expression of all ESTs in that category, according to parts per million).

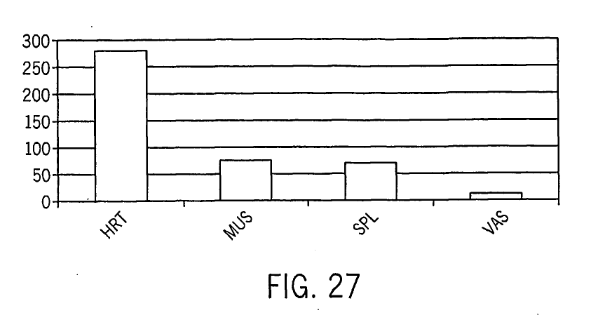

Overall, the following results were obtained as shown with regard to the histogram in FIG. 27, concerning the number of heart-specific clones in libraries/sequences.

This cluster was found to be selectively expressed in heart for the following reasons: in a comparison of the ratio of expression of the cluster in heart specific ESTs to the overall expression of the cluster in non-heart ESTs, which was found to be 16.3; the ratio of expression of the cluster in heart specific ESTs to the overall expression of the cluster in muscle-specific ESTs which was found to be 3.8; and fisher exact test P-values were computed both for library and weighted clone counts to check that the counts are statistically significant, and were found to be 2.70E-07.

One particularly important measure of specificity of expression of a cluster in heart tissue is the previously described comparison of the ratio of expression of the cluster in heart as opposed to muscle. This cluster was found to be specifically expressed in heart as opposed to non-heart ESTs as described above. However, many proteins have been shown to be generally expressed at a higher level in both heart and muscle, which is less desirable. For this cluster, as described above, the ratio of expression of the cluster in heart specific ESTs to the overall expression of the cluster in muscle-specific ESTs which was found to be 16.3, which clearly supports specific expression in heart tissue.

As noted above, cluster R15601 features 2 transcript(s), which were listed in Table 136 above. These transcript(s) encode for protein(s) which are variant(s) of protein cardiomyopathy associated 4 (SEQ. ID NO:267). A description of each variant protein according to the present invention is now provided.

Variant protein R15601_P2 (SEQ. ID NO:268) according to the present invention is encoded by transcript R15601_T8 (SEQ. ID NO:240). One or more alignments to one or more previously published cardiomyopathy associated 4 (SEQ. ID NO:267) protein sequences are given in the alignment table on the attached CD-ROM. A brief description of the relationship of the variant protein according to the present invention to each such aligned protein is as follows:

1. Comparison report between R15601_P2 (SEQ. ID NO:268) and NP_{—}775259 (SEQ. ID NO: 397):

A. An isolated chimeric polypeptide encoding for R15601P2 (SEQ. ID NO:268), comprising a first amino acid sequence being at least 70%, optionally at least 80%, preferably at least 85%, more preferably at least 90% and most preferably at least 95%, homologous to a polypeptide having the sequence MPRKDRNSSRAESAQCQVLSCVIHGILLMAREIAVVVLPLSQ (SEQ. 113 NO: 388) corresponding to amino acids 1-42 of R15601_P2 (SEQ. ID NO:268), and a second amino acid sequence being at least 90% homologous to ESYVQAASDASRAIDINSSDIKALYRRCQALEHLGKLDQAFKDVQRCATLEPRNQNFQEMLR RLNTSIQEKLRVQFSTDSRVQKMFEILLDENSEADKREKAANNLIVLGREEAGAEKIFQNNGV ALLLQLLDTKKPELVLAAVRTLSGMCSGHQARATVILHAVRIDRKSLMAVENEEMSLAVCN LLQAIIDSLSGEDKREHRGKEEALVLDTKKDLKQITSHLLDIVILVSKKVSGQGRDQALNLLNK NVPRKDLAIHDNSRTIYVVDNGLRKILKVVGQVPDLPSCLPLTDNTRMLASILINKLYDDLRC DPERDBFRKKEEYITGKFDPQDMDKNLNAIQTVSGILQGPFDLGNQLLGLKGVMEMMVALC GSERETDQLVAVEALIHASTKLSRATFIITNGVSLLKQIYKTTKNEKEKIRTLVGLCKLGSAGGT DYGLRQFAEGSTEKLAKQCRKWLCNMSIDTRTRRWAVEGLAYLTLDADVKDDFVQDVPAL QAMFELAKAGTSDKTILYSVATTLVNCTNSYDVKEVIPELVQLAKFSKQHVPEEHPKDKKDFI DMRVKRILKAGVISALACMVKADSAILTDQTKELLARVFLALCDNPKDRGTIVAQGGGKALI PLALEGTDVGKVKAAHALAKIAAVSNPDIAFPGERVYEVVRPLVRLLDTQRDGLQNYEALLG LTNLSGRSDKLRQKIFKERALPDIENYMFENHDQLRQAATECMCNMVLHKEVQERFLADGN DRLKLVVLLCGEDDDKVQNAAAGALAMLTAAHKKLCLKMTQVTTQWLEILQRLCLHDQLS VQHRGLWAYNLLAADAELAKKINESELLEILTVVGKQEPDEKKAEVYQTARECLIKCMDYG FIKPVS corresponding to amino acids 57-931 of NP_{—}775259 (SEQ. ID NO: 397), which also corresponds to amino acids 43-917 of R15601_P2 (SEQ. ID NO:268), wherein said first amino acid sequence and second amino acid sequence are contiguous and in a sequential order.

B. An isolated polypeptide encoding for a head of R15601_P2 (SEQ. ID NO:268), comprising a polypeptide being at least 70%, optionally at least about 80%, preferably at least about 85%, more preferably at least about 90% and most preferably at least about 95% homologous to the sequence MPRKDRNSSRAESAQCQVLSCVIHGILLMAREIAVVVLPLSQ (SEQ. ID NO: 388) of R15601₇P2 (SEQ. ID NO:268).

The localization of the variant protein was determined according to results from a number of different software programs and analyses, including analyses from SignalP and other specialized programs. The variant protein is believed to be membranal with regard to the cell.

Variant protein R15601_P2 (SEQ. ID NO:268) also has the following non-silent SNPs (Single Nucleotide Polymorphisms) as listed in Table 139, (given according to their position(s) on the amino acid sequence, with the alternative amino acid(s) listed.

TABLE 139
Amino acid mutations

| SNP position(s) on amino acid | |
|---|---|
| sequence | Alternative amino acid(s) |
| 46 | V -> I |
| 838 | I -> N |

Variant protein R15601_P2 (SEQ. ID NO:268) is encoded by the following transcript(s): R15601_T8 (SEQ. ID NO:240), for which the coding portion starts at position 56 and ends at position 2806. The transcript also has the following SNPs as listed in Table 140. (given according to their position on the nucleotide sequence, with the alternative nucleic acid listed.

TABLE 140
Nucleic acid SNPs

| SNP position(s) on nucleotide | |
|---|---|
| sequence | Alternative nucleic acid(s) |
| 191 | G -> A |
| 394 | G -> A |
| 1480 | C -> T |
| 1807 | C -> T |
| 2568 | T -> A |
| 2822 | A -> C |
| 2880 | G -> A |
| 2919 | A -> G |
| 2919 | A -> T |

Variant protein R15601_P3 (SEQ. ID NO:269) according to the present invention is encoded by transcript R15601_T9 (SEQ. ID NO:241). One or more alignments to one or more previously published cardiomyopathy associated 4 (SEQ. ID NO:267) protein sequences are given in the alignment table on the attached CD-ROM. A brief description of the relationship of the variant protein according to the present invention to each such aligned protein is as follows:

1. Comparison report between R15601_P3 (SEQ. ID NO:269) and NP_{—}775259 (SEQ. ID NO: 397):

A. An isolated chimeric polypeptide encoding for R15601_P3 (SEQ. ID NO:269), comprising a first amino acid sequence being at least 90% homologous to MAEVEAVQLKEEGNRHFQLQDYKAATNSYSQALKLTKDKALLATLYRNRAACGLKTESYV QAASDASRAIDINSSDIKALYRRCQALEHLGKLDQAFKDVQRCATLEPRNQNFQEMLRRLNT SIQEKLRVQFSTDSRVQKMFEILLDENSEADKREKAANNLIVLGREEAGAEKIFQNNGVALLL QLLDTKKPELVLAAVRTLSGMCSGHQARATVILHAVRIDRKSLMAVENEEMSLAVCNLLQA IIDSLSGEDKREHRGKEEALVLDTKKDLKQITSHLLDMLVSKKVSGQGRDQALNLLNKNVPR KDLAIHDNSRTIYVVDNGLRKILKVVGQVPDLPSCLPLTDNTRMLASILINKLYDDLRCDPER DHFRKKEEYITGKFDPQDMDKNLNAIQTVSGILQGPFDLGNQLLGLKGVMEMMVALCGSER ETDQLVAVEALIHASTKLSRATFIITNGVSLLKQIYKTTKNEKIKIRTLVGLCKLGSAGGIDYG LRQFAEGSTEKLAKQCRKWLCNMSIDTRTRRWAVEGLAYLTLDADVKDDFVQDVPALQAM FELAKAG corresponding to amino acids 1-565 of NP_{—}775259 (SEQ. ID NO: 397), which also corresponds to amino acids 1-565 of R15601_P3 (SEQ. ID NO:269), and a second amino acid sequence being at least 70%, optionally, at least 80%, preferably at least 85%, more preferably at least 90% and most preferably at least 95% homologous to a polypeptide having the sequence VGESGPTTNLRKGLLGPDPQGMDPSLPPGSTPYPCINMIGYFPLSGPHFT (SEQ. ID NO: 389) corresponding to amino acids 566-615 of R15601_P3 (SEQ. ID NO:269), wherein said first amino acid sequence and second amino acid sequence are contiguous and in a sequential order.

B. An isolated polypeptide encoding for an edge portion of R15601_P3 (SEQ. ID NO:269), comprising an amino acid sequence being at least 70%, optionally at least about 80%, preferably at least about 85%, more preferably at least about 90% and most preferably at least about 95% homologous to the sequence VGESGPTTNLRKGLLGPDPQGMDPSLPPGSTPYPCINMIGYFPLSGPHFT (SEQ. ID NO: 389) of R15601_P3 (SEQ. ID NO:269).

The localization of the variant protein was determined according to results from a number of different software programs and analyses, including analyses from SignalP and other specialized programs. The variant protein is believed to be located intracellularly.

Variant protein R15601_P3 (SEQ. ED NO:269) also has the following non-silent SNPs (Single Nucleotide Polymorphisms) as listed in Table 141, (given according to their position(s) on the amino acid sequence, with the alternative amino acid(s) listed.

TABLE 141
Amino acid mutations

| SNP position(s) on amino acid | |
|---|---|
| sequence | Alternative amino acid(s) |
| 60 | V -> I |

Variant protein R1560 1_P3 (SEQ. ID NO:269) is encoded by the following transcript(s): R15601_T9 (SEQ. ID NO:241), for which the coding portion starts at position 95 and ends at position 1939. The transcript also has the following SNPs as listed in Table 142 (given according to their position on the nucleotide sequence, with the alternative nucleic acid listed.

TABLE 142
Nucleic acid SNPs

| SNP position(s) on nucleotide | |
|---|---|
| sequence | Alternative nucleic acid(s) |
| 238 | C -> T |
| 272 | G -> A |
| 475 | G -> A |
| 1561 | C -> T |
| 1840 | A -> G |

As noted above, cluster R15601 features 25 segment(s), which were listed in Table 137 above and for which the sequence(s) are given. These segment(s) are portions of nucleic acid sequence(s) which are described herein separately because they are of particular interest. A description of several segments according to the present invention is now provided.

Segment cluster R15601 N6 (SEQ. ID NO:243) according to the present invention is supported by 1 libraries. The number of libraries was determined as previously described. This segment can be found in the following transcript(s): R15601 T8 (SEQ. ID NO:240). Table 143 below describes the starting and ending position of this segment on each transcript.

TABLE 143
Segment location on transcripts

| | Segment | Segment |
|---|---|---|
| Transcript name | starting position | ending position |
| R15601_T8 (SEQ. ID NO: 240) | 1 | 181 |

Segment cluster R15601 N28 (SEQ. ID NO:251) according to the present invention is supported by 1 libraries. The number of libraries was determined as previously described. This segment can be found in the following transcript(s): R1560 l_T9 (SEQ. ID NO:241). Table 144 below describes the starting and ending position of this segment on each transcript.

TABLE 144
Segment location on transcripts

| | Segment | Segment |
|---|---|---|
| Transcript name | starting position | ending position |
| R15601_T9 (SEQ. ID NO: 241) | 1790 | 1956 |

Segment cluster R15601 N30 (SEQ. ID NO:252) according to the present invention is supported by 13 libraries. The number of libraries was determined as previously described. This segment can be found in the following transcript(s): R15601_T8 (SEQ. ID NO:240). Table 145 below describes the starting and ending position of this segment on each transcript.

TABLE 145
Segment location on transcripts

| | Segment | Segment |
|---|---|---|
| Transcript name | starting position | ending position |
| R15601_T8 (SEQ. ID NO: 240) | 1709 | 1849 |

Expression of Homo sapiens cardiomyopathy associated 4 (CMYA4) R15601 transcripts which are detectable by amplicon as depicted in sequence name R15601_seg28 (SEQ. ID NO: 272) specifically in heart tissue

Expression of Homo sapiens cardiomyopathy associated 4 (CMYA4) transcripts detectable by or according to seg28-R15601_seg28 (SEQ. ID NO: 272) amplicon and primers R15601_seg28F (SEQ. ID NO: 270) and R15601_seg28R (SEQ. ID NO: 271) was measured by real time PCR. In parallel the expression of four housekeeping genes-SDHA (GenBank Accession No. NM_{—}004168 (SEQ. ID NO: 33); amplicon-SDHA-amplicon (SEQ. ID NO:36)), Ubiquitin (GenBank Accession No. BC000449 (SEQ. ID NO: 29); amplicon-Ubiquitin-amplicon (SEQ. ID NO: 32)), RPL19 (GenBank Accession No. NM_{—}000981 (SEQ. ID NO: 21); RPL19 amplicon (SEQ. ID NO: 24)) and TATA box (GenBank Accession No. NM_{—}003194 (SEQ. ID NO: 25); TATA amplicon (SEQ. ID NO: 28)) was measured similarly. For each RT sample, the expression of the above amplicon was normalized to the geometric mean of the quantities of the housekeeping genes. The normalized quantity of each RT sample was then divided by the median of the quantities of the heart samples (sample numbers 44, 45 and 46, Table 1_{—}6 above), to obtain a value of relative expression for each sample relative to median of the heart samples.

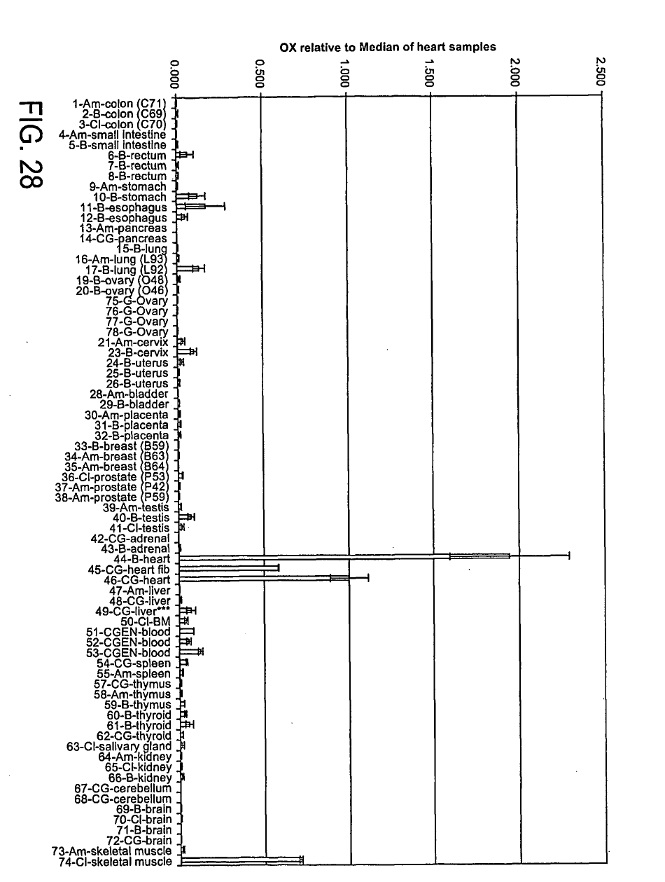

FIG. 28 is a histogram showing relative expression of the above-indicated Homo sapiens cardiomyopathy associated 4 (CMYA4) transcripts in heart tissue samples as opposed to other tissues. Values represent the average of duplicate experiments. Error bars indicate the minimal and maximal values obtained.

As is evident from FIG. 28, the expression of Homo sapiens cardiomyopathy associated 4 (CMYA4) transcripts detectable by the above amplicon in heart tissue samples was significantly higher than in most of the other samples (sample numbers 1; 2, 3, 4, 5, 6, 7, 8, 9, 10, 11, 12, 13, 14, 15, 16, 17, 19, 20, 75, 76, 77, 78, 21, 23, 24, 25, 26, 28, 29, 30, 31, 32, 33, 34, 35, 36, 37, 38, 39, 40, 41, 42, 43, 47, 48, 49, 50, 51, 52, 53, 54, 55, 57, 58, 59, 60, 61, 62, 63, 64, 65, 66, 67, 68, 69, 70, 71, 72, 73 and 74, Table 1_{—}6 above).

Primer pairs are also optionally and preferably encompassed within the present invention; for example, for the above experiment, the following primer pair was used as a non-limiting illustrative example only of a suitable primer pair: R15601_seg28F (SEQ. ID NO: 270) forward primer; and R15601_seg28R (SEQ. ID NO: 271) reverse primer.

The present invention also preferably encompasses any amplicon obtained through the use of any suitable primer pair; for example, for the above experiment, the following amplicon was obtained as a non-limiting illustrative example only of a suitable amplicon: R15601_seg28 (SEQ. ID NO: 272).

Forward Primer (R15601_seg28F
(SEQ. ID NO: 270)): GTCTGGCCCGACCACAAAC
Reverse Primer (R15601_seg28R
(SEQ. ID NO: 271)): GGTAAGGAGTAGAGCCAGGAGGA
Amplicon (R15601_seg28
(SEQ. ID NO: 272)): GTCTGGCCCGACCACAAACCTCAGGAAAGG
TCTGCTGGGTCCAGACCCACAGGGAATGGATCCCAGTCTTCCTCCTGGCT
CTACTCCTTACC

Expression of Homo sapiens cardiomyopathy associated 4 (CMYA4) R15601 transcripts which are detectable by amplicon as depicted in sequence name R15601_seg30WT (SEQ. ID NO: 275) specifically in heart tissue

Expression of Homo sapiens cardiomyopathy associated 4 (CMYA4) transcripts detectable by or according to seg30WT-R15601_seg30WT (SEQ. 1513 NO: 275) amplicon and primers R15601_seg30WTF (SEQ. ID NO: 273) and R15601_seg30WTR (SEQ. ID NO: 274) was measured by real time PCR. In parallel the expression of four housekeeping genes-SDHA (GenBank Accession No. NM_{—}004168 (SEQ. ID NO. 33); amplicon-SDHA-amplicon (SEQ. ID NO:36)), Ubiquitin (GenBank Accession No. BC000449 (SEQ. ID NO: 29); amplicon-Ubiquitin-amplicon (SEQ. ID NO: 32)), RPL19 (GenBank Accession No. NM_{—}000981 (SEQ. ID NO: 21); RPL19 amplicon (SEQ. ID NO: 24)) and TATA box (GenBank Accession No. NM_{—}003194 (SEQ. ID NO: 25); TATA amplicon (SEQ. ID NO: 28)) was measured similarly. For each RT sample, the expression of the above amplicon was normalized to the geometric mean of the quantities of the housekeeping genes. The normalized quantity of each RT sample was then divided by the median of the quantities of the heart samples (sample numbers 44, 45 and 46, Table 1_{—}6 above), to obtain a value of relative expression for each sample relative to median of the heart samples.

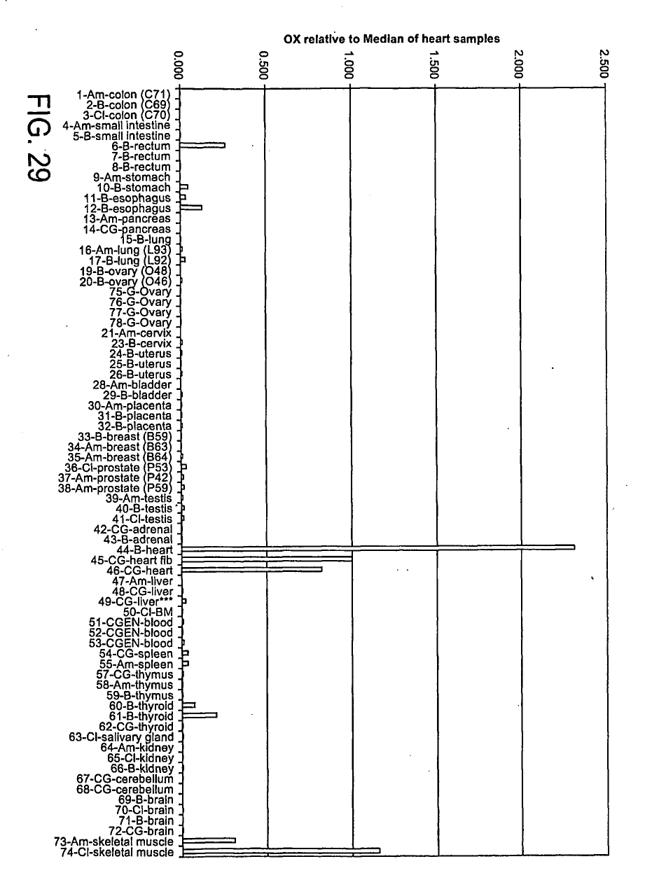

FIG. 29 is a histogram showing relative expression of the above-indicated Homo sapiens cardiomyopathy associated 4 (CMYA4) transcripts in heart tissue samples as opposed to other tissues.

As is evident from FIG. 29, the expression of Homo sapiens cardiomyopathy associated 4 (CMYA4) transcripts detectable by the above amplicon in heart tissue samples was significantly higher than in most of the other samples (sample numbers 1, 2, 3, 4, 5, 6, 7, 8, 9, 10, 11, 12, 13, 14, 15, 16, 17, 19, 20, 75, 76, 77, 78, 21, 23, 24, 25, 26, 28, 29, 30, 31, 32, 33, 34, 35, 36, 37, 38, 39, 40, 41, 42, 43, 47, 48, 49, 50, 51, 52, 53, 54, 55, 57, 58, 59, 60, 61, 62, 63, 64, 65, 66, 67, 68, 69, 70, 71, 72, 73 and 74, Table 1_{—}6 above).

Primer pairs are also optionally and preferably encompassed within the present invention; for example, for the above experiment, the following primer pair was used as a non-limiting illustrative example only of a suitable primer pair: R15601_seg30WTF (SEQ. ID NO: 273) forward primer; and R15601_seg30WTR (SEQ. ID NO: 274) reverse primer.

The present invention also preferably encompasses any amplicon obtained through the use of any suitable primer pair; for example, for the above experiment, the following amplicon was obtained as a non-limiting illustrative example only of a suitable amplicon: R15601_seg30WT (SEQ. ID NO: 275).

Forward Primer (R15601_seg30WTF
(SEQ. ID NO: 273)): ACCATCCTGTACTCGGTGGC
Reverse Primer (R15601_seg30WTR
(SEQ. ID NO: 274)): CATGCTGCTTGGAGAACTTGG
Amplicon (R15601_seg30WT
(SEQ. ID NO: 275)): ACCATCCTGTACTCGGTGGCCACCACCCTG
GTGAACTGCACCAACAGCTACGATGTCAAGGAGGTCATCCCAGAGCTT
GTCCAGCTCGCCAAGTTCTCCAAGCAGCATG

### Description for Cluster T11811

Cluster T11811 features 6 transcript(s) and 20 segment(s) of interest, the names for which are given in Tables 146 and 147, respectively. The selected protein variants are given in table 148.

TABLE 146
Transcripts of interest
Transcript Name

T11811_T3 (SEQ. ID NO: 276)
T11811_T6 (SEQ. ID NO: 277)
T11811_T12 (SEQ. ID NO: 278)
T11811_T13 (SEQ. ID NO: 279)
T11811_T15 (SEQ. ID NO: 280)
T11811_T24 (SEQ. ID NO: 281)

TABLE 147
Segments of interest
Segment Name

T11811_N0 (SEQ. ID NO: 282)
T11811_N10 (SEQ. ID NO: 283)
T11811_N26 (SEQ. ID NO: 284)
T11811_N2 (SEQ. ID NO: 285)
T11811_N4 (SEQ. ID NO: 286)
T11811_N5 (SEQ. ID NO: 287)
T11811_N7 (SEQ. ID NO: 288)
T11811_N8 (SEQ. ID NO: 289)
T11811_N9 (SEQ. ID NO: 290)
T11811_N11 (SEQ. ID NO: 291)
T11811_N12 (SEQ. ID NO: 292)
T11811_N13 (SEQ. ID NO: 293)
T11811_N14 (SEQ. ID NO: 294)
T11811_N15 (SEQ. ID NO: 295)
T11811_N17 (SEQ. ID NO: 296)
T11811_N18 (SEQ. ID NO: 297)
T11811_N20 (SEQ. ID NO: 298)
T11811_N21 (SEQ. ID NO: 299)
T11811_N22 (SEQ. ID NO: 300)
T11811_N23 (SEQ. ID NO: 301)

TABLE 148
Proteins of interest

| Protein Name | Corresponding Transcript(s) |
|---|---|
| T11811_P2 (SEQ. ID NO: 303) | T11811_T3 (SEQ. ID NO: 276) |
| T11811_P4 (SEQ. ID NO: 304) | T11811_T6 (SEQ. ID NO: 277) |
| T11811_P7 (SEQ. ID NO: 305) | T11811_T12 (SEQ. ID NO: 278) |
| T11811_P8 (SEQ. ID NO: 306) | T11811_T13 (SEQ. ID NO: 279) |
| T11811_P10 (SEQ. ID NO: 307) | T11811_T15 (SEQ. ID NO: 280) |
| T11811_P15 (SEQ. ID NO: 308) | T11811_T24 (SEQ. ID NO: 281) |

These sequences are variants of the known protein Myosin regulatory light chain 2 (SEQ. ID NO:302), atrial isoform (SwissProt accession identifier MLRA_HUMAN (SEQ. ID NO: 398); known also according to the synonyms Myosin light chain 2a; MLC-2a; MLC2a; Myosin regulatory light chain 7), referred to herein as the previously known protein.

The sequence for protein Myosin regulatory light chain 2 (SEQ. ID NO:302), atrial isoform is given.

According to optional but preferred embodiments of the present invention, variants of this cluster according to the present invention (amino acid and/or nucleic acid sequences of T11811) may optionally have one or more of the following utilities, as described in greater detail below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

A non-limiting example of such a utility is the detection, diagnosis and/or determination of dilated cardiomyopathy (DCM). The method comprises detecting a T11811 variant, for example a variant protein, protein fragment, peptide, polynucleotide, polynucleotide fragment and/or oligonucleotide as described herein, optionally and preferably in a serum sample. The expression levels of the T11811 variant as determined in a patient can be further compared to those in a normal individual.

Differential expression of the known Myosin regulatory light chain 2 (SEQ. ID NO:302), atrial isoform is described with regard to PCT Application No. WO 03/040407, hereby incorporated by reference as if fully set forth herein. Differential expression was measured in samples taken from healthy cardiac tissue and also from samples taken from patients suffering from DCM, using total or amplified RNA. According to other optional embodiments of the present invention, variants of this cluster according to the present invention (amino acid and/or nucleic acid sequences of T11811) may optionally have one or more of the following utilities, some of which are related to utilities described above. It should be noted that these utilities are optionally and preferably suitable for human and non-human animals as subjects, except where otherwise noted.

The Table below (Table 149) describes diagnostic utilities for the cluster T11811 that were found through microarrays, including the statistical significance thereof and a reference. One or more T11811 variants according to the present invention may optionally have one or more of these utilities.

TABLE 149
Microarray data for T11811

| | |
|---|---|
| identification of heart cellular | jp_atlas, GNF1, GNF2, |
| damage, due to very high | med_all_avg (internal |
| expression in heart. | database). |

The following GO Annotation(s) apply to the previously known protein. The following annotation(s) were found: actin filament-based movement; smooth muscle contraction, which are annotation(s) related to Biological Process; ATPase activity, coupled; calcium ion binding; microfilament motor activity, which are annotation(s) related to Molecular Function; and myosin, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

As noted above, cluster T11811 features 6 transcript(s), which were listed in Table 146 above. These transcript(s) encode for protein(s) which are variant(s) of protein Myosin regulatory light chain 2 (SEQ. ID NO:302), atrial isoform. A description of each variant protein according to the present invention is now provided.

Variant protein T11811_P2 (SEQ. ID NO:303) according to the present invention is encoded by transcript T11811_T3 (SEQ. ID NO:276). One or more alignments to one or more previously published Myosin regulatory light chain 2 (SEQ. ID NO:302) protein sequences are given in the alignment table on the attached CD-ROM. A brief description of the relationship of the variant protein according to the present invention to each such aligned protein is as follows:

1. Comparison report between T11811_P2 (SEQ. ID NO:303) and MLRA_HUMAN (SEQ. ID NO: 398):

A. An isolated chimeric polypeptide encoding for T11811_P2 (SEQ. ID NO:303), comprising a first amino acid sequence being at least 90% homologous to MASRKAGTRGKVAATKQAQRGSSNVFSMFEQAQIQEFKEAFSCIDQNRDGIICKADLRETYS QLGKVSVPEEELDAMLQEGKGPINFIVFLTLFGEKLNGTDPEEAILSAFRMFDPSGKGVVNKD EFKQLLLTQADKFSPAE corresponding to amino acids 1,-142 of MLRA_HUMAN (SEQ. ID NO: 398), which also corresponds to amino acids 1-142 of T11811_P2 (SEQ. ID NO:303), a second amino acid sequence being at least 70%, optionally at least 80%, preferably at least 85%, more preferably at least 90% and most preferably at least 95% homologous to a polypeptide having the sequence VRLPSPFNTHPQHLLWAFTHDPEPSTSEAVAGR (SEQ. ID NO: 390) corresponding to amino acids 143-175 of T11811_P2 (SEQ. ID NO:303), and a third amino acid sequence being at least 90% homologous to VEQMFALTPMDLAGNIDYKSLCYIITHGDEKEE corresponding to amino acids 143-175 of MLRA_HUMAN (SEQ. ID NO: 398), which also corresponds to amino acids 176-208 of T11811_P2 (SEQ. ID NO:303), wherein said first amino acid sequence, second amino acid sequence and third amino acid sequence are contiguous and in a sequential order.

B. An isolated polypeptide encoding for an edge portion of T11811_P2 (SEQ. ID NO:303), comprising an amino acid sequence being at least 70%, optionally at least about 80%, preferably at least about 85%, more preferably at least about 90% and most preferably at least about 95% homologous to the sequence VRLPSPFNTHPQHLLWAFTHDPEPSTSEAVAGR (SEQ. ID NO: 390) of T11811_P2 (SEQ. ID NO:303).

The localization of the variant protein was determined according to results from a number of different software programs and analyses, including analyses from SignalP and other specialized programs. The variant protein is believed to be located intracellularly.

Variant protein T11811_P2 (SEQ. 113 NO:303) also has the following non-silent SNPs (Single

Nucleotide Polymorphisms) as listed in Table 150, (given according to their position(s) on the amino acid sequence, with the alternative amino acid(s) listed.

TABLE 150
Amino acid mutations

| SNP position(s) on | |
|---|---|
| amino acid | Alternative |
| sequence | amino acid(s) |
| 8 | T -> |
| 37 | F -> |
| 54 | K -> |
| 58 | R -> |
| 69 | V -> |
| 77 | M -> I |
| 82 | K -> |
| 90 | F -> |
| 97 | K -> * |
| 97 | K -> |
| 102 | D -> |
| 105 | E -> |
| 118 | G -> R |

Variant protein T11811_P2 (SEQ. ID NO:303) is encoded by the following transcript(s): T11811_T3 (SEQ. ID NO:276), for which the coding portion starts at position 347 and ends at position. 970. The transcript also has the following SNPs as listed in Table 151 (given according to their position on the nucleotide sequence, with the alternative nucleic acid listed.

TABLE 151
Nucleic acid SNPs

| SNP position(s) | |
|---|---|
| on nucleotide | |
| sequence | Alternative nucleic acid(s) |
| 234 | A -> |
| 237 | G -> A |
| 319 | C -> T |
| 369 | C -> |
| 371 | C -> A |
| 455 | T -> |
| 463 | A -> G |
| 506 | A -> |
| 519 | G -> |
| 553 | C -> |
| 553 | C -> T |
| 577 | G -> T |
| 592 | G -> |
| 613 | C -> T |
| 616 | C -> |
| 635 | A -> |
| 635 | A -> T |
| 652 | C -> |
| 660 | A -> |
| 698 | G -> C |
| 988 | C -> |

Variant protein T11811_P4 (SEQ. ID NO:304) according to the present invention is encoded by transcript T11811_T6 (SEQ. ID NO:277). One or more alignments to one or more previously published Myosin regulatory light chain 2 (SEQ. ID NO:302) protein sequences are given in the alignment table on the attached CD-ROM. A brief description of the relationship of the variant protein according to the present invention to each such aligned protein is as follows:

1. Comparison report between T11811_P4 (SEQ. ID NO:304) and MLRA_HUMAN (SEQ. ID NO: 398):

A. An isolated chimeric polypeptide encoding for T11811_P4 (SEQ. ID NO:304), comprising a first amino acid sequence being at least 90% homologous to MASRKAGTRGKVAATKQAQRGSSNVFSMFEQAQIQEFKEAFSCIDQNRDGIIKCADLRETYS QLGKVSVPEEELDAMLQEGKGPINFTVFLTLFGEKLNGTDPEEAILSAFRIVTDPSGKGVVNKD corresponding to amino acids 1-125 of MLRA_HUMAN (SEQ. ID NO: 398), which also corresponds to amino acids 1-125 of T11811_P4 (SEQ. 11) NO:304), and a second amino acid sequence being at least 70%, optionally at least 80%, preferably at least 85%, more preferably at least 90% and most preferably at least 95% homologous to a polypeptide having the sequence DQPFPAPWEPPYPPSLCSHSPAVSCSDPPHPPGSSSFS (SEQ. ID NO: 391) corresponding to amino acids 126-163 of T11811_P4 (SEQ. ID NO:304), wherein said first amino acid sequence and second amino acid sequence are contiguous and in a sequential order.

B. An isolated polypeptide encoding for an edge portion of T11811_P4 (SEQ. ID NO:304), comprising an amino acid sequence being at least 70%, optionally at least about 80%, preferably at least about 85%, more preferably at least about 90% and most preferably at least about 95% homologous to the sequence DQPFPAPWEPPYPPSLCSHSPAVSCSDPPHPPGSSSFS (SEQ. ID NO: 391) of T11811_P4 (SEQ. ID NO:304).

The localization of the variant protein was determined according to results from a number of different software programs and analyses, including analyses from SignalP and other specialized programs. The variant protein is believed to be located intracellularly.

Variant protein T11811_P4 (SEQ. ID NO:304) also has the following non-silent SNPs (Single Nucleotide Polymorphisms) as listed in Table 152, (given according to their position(s) on the amino acid sequence, with the alternative amino acid(s) listed.

TABLE 152
Amino acid mutations

| SNP position(s) on | Alternative |
|---|---|
| amino acid sequence | amino acid(s) |
| 8 | T -> |
| 37 | F -> |
| 54 | K -> |
| 58 | R -> |
| 69 | V -> |
| 77 | M -> I |
| 82 | K -> |
| 90 | F -> |
| 97 | K -> * |
| 97 | K -> |
| 102 | D -> |
| 105 | E -> |
| 118 | G -> R |
| 138 | P -> S |
| 140 | S -> T |

Variant protein T11811_P4 (SEQ. ID NO:304) is encoded by the following transcript(s): T11811_T6 (SEQ. ID NO:277), for which the coding portion starts at position 347 and ends at position 835. The transcript also has the following SNPs as listed in Table 153 (given according to their position on the nucleotide sequence, with the alternative nucleic acid listed.

TABLE 153
Nucleic acid SNPs

| SNP position(s) on | Alternative |
|---|---|
| nucleotide sequence | nucleic acid(s) |
| 234 | A -> |
| 237 | G -> A |
| 319 | C -> T |
| 369 | C -> |
| 371 | C -> A |
| 455 | T -> |
| 463 | A -> G |
| 506 | A -> |
| 519 | G -> |
| 553 | C -> |
| 553 | C -> T |
| 577 | G -> T |
| 592 | G -> |
| 613 | C -> T |
| 616 | C -> |
| 635 | A -> |
| 635 | A -> T |
| 652 | C -> |
| 660 | A -> |
| 698 | G -> C |
| 758 | C -> T |
| 764 | T -> A |
| 984 | C -> |

Variant protein T11811_P7 (SEQ. ID NO:305) according to the present invention is encoded by transcript T11811_T12 (SEQ. ID NO:278). One or more alignments to one or more previously published Myosin regulatory light chain 2 (SEQ. ID NO:302) protein sequences are given in the alignment table on the attached CD-ROM. A brief description of the relationship of the variant protein according to the present invention to each such aligned protein is as follows:

1. Comparison report between T11811_P7 (SEQ. ID NO:305) and MLRA_HUMAN (SEQ. ID NO: 398):

A. An isolated chimeric polypeptide encoding for T11811_P7 (SEQ. ID NO:305), comprising a first amino acid sequence being at least 90% homologous to MASRKAGTRGKVAATKQAQRGSSNVFSMFEQAQIQEFKE corresponding to amino acids 1-39 of MLRA_HUMAN (SEQ. ID NO: 398), which also corresponds to amino acids 1-39 of T118111_P7 (SEQ. ID NO:305), a second amino acid sequence being at least 70%, optionally at least 80%, preferably at least 85%, more preferably at least 90% and most preferably at least 95% homologous to a polypeptide having the sequence VSPPPPTFPRAGGCSHLKAPIPQ (SEQ. ID NO: 392) corresponding to amino acids 40-62 of T11811_P7 (SEQ. ID NO:305), and a third amino acid sequence being at least 90% homologous to AFSCIDQNRDGIICKADLRETYSQLGKVSVPEEELDAMLQEGKGPINFTVFLTLFGEKLNGTDP EEAILSAFRMFDPSGKGVVNKDEFKQLLLTQADKFSPAEVEQMFALTPMDLAGNIDYKSLCYI ITHGDEKEE corresponding to amino acids 40-175 of MLRA_HUMAN (SEQ. ID NO: 398), which also corresponds to amino acids 63-198 of T11811_P7 (SEQ. ID NO:305), wherein said first amino acid sequence, second amino acid sequence and third amino acid sequence are contiguous and, in a sequential order.

B. An isolated polypeptide encoding for an edge portion of T11811_P7 (SEQ. ID NO:305), comprising an amino acid sequence being at least 70%, optionally at least about 80%, preferably at least about 85%, more preferably at least about 90% and most preferably at least about 95% homologous to the sequence VSPPPPTFPRAGGCSHLKAPIPQ (SEQ. ID NO: 392) of T11811_P7 (SEQ. ID NO:305).

The localization of the variant protein was determined according to results from a number of different software programs and analyses, including analyses from SignalP and other specialized programs. The variant protein is believed to be located intracellularly.

Variant protein T11811_P7 (SEQ. ID NO:305) also has the following non-silent SNPs (Single Nucleotide Polymorphisms) as listed in Table 154, (given according to their position(s) on the amino acid sequence, with the alternative amino acid(s) listed.

TABLE 154
Amino acid mutations

| SNP position(s) on amino | Alternative |
|---|---|
| acid sequence | amino acid(s) |
| 8 | T -> |
| 37 | F -> |
| 77 | K -> |
| 81 | R -> |
| 92 | V -> |
| 100 | M -> I |
| 105 | K -> |
| 113 | F -> |
| 120 | K -> * |
| 120 | K -> |
| 125 | D -> |
| 128 | E -> |
| 141 | G -> R |

Variant protein T11811_P7 (SEQ. ID NO:305) is encoded by the following transcript(s): T11811_T12 (SEQ. ID NO:278), for which the coding portion starts at position 347 and ends at position 940. The transcript also has the following SNPs as listed in Table 155 (given according to their position on the nucleotide sequence, with the alternative nucleic acid listed.

TABLE 155
Nucleic acid SNPs

| SNP position(s) on | Alternative |
|---|---|
| nucleotide sequence | nucleic acid(s) |
| 234 | A -> |
| 237 | G -> A |
| 319 | C -> T |
| 369 | C -> |
| 371 | C -> A |
| 455 | T -> |
| 463 | A -> G |
| 575 | A -> |
| 588 | G -> |
| 622 | C -> |
| 622 | C -> T |
| 646 | G -> T |
| 661 | G -> |
| 682 | C -> T |
| 685 | C -> |
| 704 | A -> |
| 704 | A -> T |
| 721 | C -> |
| 729 | A -> |
| 767 | G -> C |
| 958 | C -> |

Variant protein T11811_P8 (SEQ. ID NO:306) according to the present invention is encoded by transcript T11811_T13 (SEQ. ID NO:279). One or more alignments to one or more previously published Myosin regulatory light chain 2 (SEQ. ID NO:302) protein sequences are given in the alignment table on the attached CD-ROM. A brief description of the relationship of the variant protein according to the present invention to each such aligned protein is as follows:

1. Comparison report between T11811_P8 (SEQ. ID NO:306) and MLRA_HUMAN (SEQ. ID NO: 398):

A. An isolated chimeric polypeptide encoding for T118111_P8 (SEQ. ID NO:306), comprising a first amino acid sequence being at least 90% homologous to MASRKAGTRGKVAATKQAQRGSSNVFSMFEQAQIQEFKEAFSCIDQNRDGIICKADLRETYS QLGKVSVPEEELDAMLQEGKGPINFTVFLTLFGEKLNGTDPEEAILSAFRMFDPSGKGVVNKD E corresponding to amino acids 1-126 of MLRA_HUMAN (SEQ. ID NO: 398), which also corresponds to amino acids 1-126 of T11811_P8 (SEQ. ID NO:306), and a second amino acid sequence being at least 70%, optionally at least 80%, preferably at least 85%, more preferably at least 90% and most preferably at least 95% homologous to a polypeptide having the sequence WSRCSP (SEQ. ID NO: 393) corresponding to amino acids 127-132 of T11811_P8 (SEQ. ID NO:306), wherein said first amino acid sequence and second amino acid sequence are contiguous and in a sequential order.

B. An isolated polypeptide encoding for an edge portion of T11811_P8 (SEQ. ID NO:306), comprising an amino acid sequence being at least 70%, optionally at least about 80%, preferably at least about 85%, more preferably at least about 90% and most preferably at least about 95% homologous to the sequence WSRCSP (SEQ. ID NO: 393) of T11811_P8 (SEQ. ID NO:306).

The localization of the variant protein was determined according to results from a number of different software programs and analyses, including analyses from SignalP and other specialized programs. The variant protein is believed to be located intracellularly.

Variant protein T11811_P8 (SEQ. ID NO:306) also has the following non-silent SNPs (Single Nucleotide Polymorphisms) as listed in Table 156, (given according to their position(s) on the amino acid sequence, with the alternative amino acid(s) listed.

TABLE 156
Amino acid mutations

| SNP position(s) on amino | Alternative |
|---|---|
| acid sequence | amino acid(s) |
| 8 | T -> |
| 37 | F -> |
| 54 | K -> |
| 58 | R -> |
| 69 | V -> |
| 77 | M -> I |
| 82 | K -> |
| 90 | F -> |
| 97 | K -> * |
| 97 | K -> |
| 102 | D -> |
| 105 | E -> |
| 118 | G -> R |

Variant protein T11811_P8 (SEQ. ID NO:306) is encoded by the following transcript(s): T11811_T13 (SEQ. ID NO:279), for which the coding portion starts at position 347 and ends at position 742. The transcript also has the following SNPs as listed in Table 157 (given according to their position on the nucleotide sequence, with the alternative nucleic acid listed.

TABLE 157
Nucleic acid SNPs

| SNP position(s) on | Alternative |
|---|---|
| nucleotide sequence | nucleic acid(s) |
| 234 | A -> |
| 237 | G -> A |
| 319 | C -> T |
| 369 | C -> |
| 371 | C -> A |
| 455 | T -> |
| 463 | A -> G |
| 506 | A -> |
| 519 | G -> |
| 553 | C -> |
| 553 | C -> T |
| 577 | G -> T |
| 592 | G -> |
| 613 | C -> T |
| 616 | C -> |
| 635 | A -> |
| 635 | A -> T |
| 652 | C -> |
| 660 | A -> |
| 698 | G -> C |
| 840 | C -> |

Variant protein T11811_P10 (SEQ. ID NO:307) according to the present invention is encoded by transcript T11811_T15 (SEQ. ID NO:280).

The localization of the variant protein was determined according to results from a number of different software programs and analyses, including analyses from SignalP and other specialized programs. The variant protein is believed to be located intracellularly.

Variant protein T11811_P10 (SEQ. ID NO:307) also has the following non-silent SNPs (Single Nucleotide Polymorphisms) as listed in Table 158, (given according to their position(s) on the amino acid sequence, with the alternative amino acid(s) listed.

TABLE 158
Amino acid mutations

| SNP position(s) on amino | Alternative |
|---|---|
| acid sequence | amino acid(s) |
| 8 | T -> |
| 37 | F -> |
| 54 | K -> |
| 58 | R -> |
| 77 | P -> |
| 100 | P -> |
| 100 | P -> S |
| 108 | A -> S |
| 113 | G -> |
| 120 | L -> F |
| 121 | P -> |
| 127 | E -> |
| 127 | E -> V |
| 133 | P -> |
| 135 | G -> |

Variant protein T11811_P10 (SEQ. ID NO:307) is encoded by the following transcript(s): T11811_T15 (SEQ. ID NO:280), for which the coding portion starts at position 347 and ends at position 778. The transcript also has the following SNPs as listed in Table 159 (given according to their position on the nucleotide sequence, with the alternative nucleic acid listed.

TABLE 159
Nucleic acid SNPs

| SNP position(s) on | Alternative |
|---|---|
| nucleotide sequence | nucleic acid(s) |
| 234 | A -> |
| 237 | G -> A |
| 319 | C -> T |
| 369 | C -> |
| 371 | C -> A |
| 455 | T -> |
| 463 | A -> G |
| 506 | A -> |
| 519 | G -> |
| 576 | C -> |
| 577 | C -> |
| 644 | C -> |
| 644 | C -> T |
| 668 | G -> T |
| 683 | G -> |
| 704 | C -> T |
| 707 | C -> |
| 726 | A -> |
| 726 | A -> T |
| 743 | C -> |
| 751 | A -> |
| 789 | G -> C |
| 980 | C -> |

Variant protein T11811_P15 (SEQ. ID NO:308) according to the present invention is encoded by transcript T11811_T24 (SEQ. ID NO:281).

The localization of the variant protein was determined according to results from a number of different software programs and analyses, including analyses from SignalP and other specialized programs. The variant protein is believed to be located intracellularly.

Variant protein T11811_P15 (SEQ. ID NO:308) also has the following non-silent SNPs (Single Nucleotide Polymorphisms) as listed in Table 160, (given according to their position(s) on the amino acid sequence, with the alternative amino acid(s) listed.

TABLE 160
Amino acid mutations

| SNP | |
|---|---|
| position(s) on | Alternative |
| amino acid | amino |
| sequence | acid(s) |
| 8 | T -> |
| 37 | F -> |
| 77 | K -> |
| 81 | R -> |

Variant protein T11811_P15 (SEQ. ID NO:308) is encoded by the following transcript(s): T11811_T24 (SEQ. ID NO:281), for which the coding portion starts at position 347 and ends at position 652. The transcript also has the following SNPs as listed in Table 161 (given according to their position on the nucleotide sequence, with the alternative nucleic acid listed.

TABLE 161
Nucleic acid SNPs

| SNP | |
|---|---|
| position(s) on | Alternative |
| nucleotide | nucleic |
| sequence | acid(s) |
| 234 | A -> |
| 237 | G -> A |
| 319 | C -> T |
| 369 | C -> |
| 371 | C -> A |
| 455 | T -> |
| 463 | A -> G |
| 575 | A -> |
| 588 | G -> |
| 693 | T -> C |
| 736 | C -> T |
| 758 | C -> G |
| 758 | C -> T |
| 839 | C -> |
| 840 | C -> |
| 907 | C -> |
| 907 | C -> T |
| 931 | G -> T |
| 946 | G -> |
| 967 | C -> T |
| 970 | C -> |
| 989 | A -> |
| 989 | A -> T |
| 1006 | C -> |
| 1014 | A -> |
| 1052 | G -> C |
| 1243 | C -> |

As noted above, cluster T11811 features 20 segment(s), which were listed in Table 147 above and for which the sequence(s) are given. These segment(s) are portions of nucleic acid sequence(s) which are described herein separately because they are of particular interest. A description of several segments according to the present invention is now provided.

Segment cluster T11811_N10 (SEQ. ID NO:283) according to the present invention is supported by 17 libraries. The number of libraries was determined as previously described. This segment can be found in the following transcript(s): T11811_T24 (SEQ. ID NO:281). Table 162 below describes the starting and ending position of this segment on each transcript.

TABLE 162
Segment location on transcripts

| | Segment | Segment |
|---|---|---|
| | starting | ending |
| Transcript name | position | position |
| T11811_T24 (SEQ. ID NO: 281) | 609 | 802 |

Segment cluster T11811_N26 (SEQ. ID NO:284) according to the present invention is supported by 54 libraries. The number of libraries was determined as previously described. This segment can be found in the following transcript(s): T11811_T12 (SEQ. ID NO:278), T11811_T13 (SEQ. ID NO:279), T11811_T15 (SEQ. ID NO:280), T11811_T24 (SEQ. ID NO:281), T11811_T3 (SEQ. ID NO:276) and T11811_T6 (SEQ. ID NO:277). Table 163 below describes the starting and ending position of this segment on each transcript.

TABLE 163
Segment location on transcripts

| | Segment | Segment |
|---|---|---|
| | starting | ending |
| Transcript name | position | position |
| T11811_T12 (SEQ. ID NO: 278) | 842 | 999 |
| T11811_T13 (SEQ. ID NO: 279) | 724 | 881 |
| T11811_T15 (SEQ. ID NO: 280) | 864 | 1021 |
| T11811_T24 (SEQ. ID NO: 281) | 1127 | 1284 |
| T11811_T3 (SEQ. ID NO: 276) | 872 | 1029 |
| T11811_T6 (SEQ. ID NO: 277) | 868 | 1025 |

According to an optional embodiment of the present invention, short segments related to the above cluster are also provided. These segments are up to about 120 by in length, and so are included in a separate description.

Segment cluster T11811_N7 (SEQ. ID NO:288) according to the present invention is supported by 22 libraries. The number of libraries was determined as previously described. This segment can be found in the following transcript(s): T11811_T12 (SEQ. ID NO:278) and T11811_T24 (SEQ. ID NO:281). Table 164 below describes the starting and ending position of this segment on each transcript.

TABLE 164
Segment location on transcripts

| | Segment | Segment |
|---|---|---|
| | starting | ending |
| Transcript name | position | position |
| T11811_T12 (SEQ. ID NO: 278) | 464 | 492 |
| T11811_T24 (SEQ. ID NO: 281) | 464 | 492 |

Segment cluster T11811_N8 (SEQ. ID NO:289) according to the present invention is supported by 26 libraries. The number of libraries was determined as previously described. This segment can be found in the following transcript(s): T11811_T12 (SEQ. ID NO:278) and T11811_T24 (SEQ. ID NO:281). Table 165 below describes the starting and ending position of this segment on each transcript.

TABLE 165
Segment location on transcripts

| | Segment | Segment |
|---|---|---|
| | starting | ending |
| Transcript name | position | position |
| T11811_T12 (SEQ. ID NO: 278) | 493 | 532 |
| T11811_T24 (SEQ. ID NO: 281) | 493 | 532 |

Segment cluster T11811_N9 (SEQ. ID NO:290) according to the present invention is supported by 64 libraries. The number of libraries was determined as previously described. This segment can be found in the following transcript(s): T11811_T12 (SEQ. ID NO:278), T11811_T13 (SEQ. ID NO:279), T11811_T15 (SEQ. ID NO:280), T11811_T24 (SEQ. ID NO:281), T11811_T3 (SEQ. ID NO:276) and T11811_T6 (SEQ. ID NO:277). Table 166 below describes the starting and ending position of this segment on each transcript.

TABLE 166
Segment location on transcripts

| | Segment | Segment |
|---|---|---|
| | starting | ending |
| Transcript name | position | position |
| T11811_T12 (SEQ. ID NO: 278) | 533 | 608 |
| T11811_T13 (SEQ. ID NO: 279) | 464 | 539 |
| T11811_T15 (SEQ. ID NO: 280) | 464 | 539 |
| T11811_T24 (SEQ. ID NO: 281) | 533 | 608 |
| T11811_T3 (SEQ. ID NO: 276) | 464 | 539 |
| T11811_T6 (SEQ. ID NO: 277) | 464 | 539 |

Segment cluster T11811_N11 (SEQ. ID NO:291) according to the present invention is supported by 14 libraries. The number of libraries was determined as previously described. This segment can be found in the following transcript(s): T11811_T15 (SEQ. ID NO:280) and T11811_T24 (SEQ. ID NO:281). Table 167 below describes the starting and ending position of this segment on each transcript.

TABLE 167
Segment location on transcripts

| | Segment | Segment |
|---|---|---|
| | starting | ending |
| Transcript name | position | position |
| T11811_T15 (SEQ. ID NO: 280) | 540 | 563 |
| T11811_T24 (SEQ. ID NO: 281) | 803 | 826 |

Segment cluster T11811_N12 (SEQ. ID NO:292) according to the present invention is supported by 13 libraries. The number of libraries was determined as previously described. This segment can be found in the following transcript(s): T11811_T15 (SEQ. ID NO:280) and T11811_T24 (SEQ. ID NO:281). Table 168 below describes the starting and ending position of this segment on each transcript.

TABLE 168
Segment location on transcripts

| | Segment | Segment |
|---|---|---|
| | starting | ending |
| Transcript name | position | position |
| T11811_T15 (SEQ. ID NO: 280) | 564 | 600 |
| T11811_T24 (SEQ. ID NO: 281) | 827 | 863 |

Segment cluster T11811_N13 (SEQ. ID NO:293) according to the present invention is supported by 10 libraries. The number of libraries was determined as previously described. This segment can be found in the following transcript(s): T11811_T15 (SEQ. ID NO:280) and T11811_T24 (SEQ. ID NO:281). Table 169 below describes the starting and ending position of this segment on each transcript.

TABLE 169
Segment location on transcripts

| | Segment | Segment |
|---|---|---|
| | starting | ending |
| Transcript name | position | position |
| T11811_T15 (SEQ. ID NO: 280) | 601 | 630 |
| T11811_T24 (SEQ. ID NO: 281) | 864 | 893 |

Segment cluster T11811_N14 (SEQ. ID NO:294) according to the present invention is supported by 57 libraries. The number of libraries was determined as previously described. This segment can be found in the following transcript(s): T11811_T12 (SEQ. ID NO:278), T11811_T13 (SEQ. ID NO:279), T11811_T15 (SEQ. ID NO:280), T11811_T24 (SEQ. ID NO:281), T11811_T3 (SEQ. ID NO:276) and T11811_T6 (SEQ. ID NO:277). Table 170 below describes the starting and ending position of this segment on each transcript.

TABLE 170
Segment location on transcripts

| | Segment | Segment |
|---|---|---|
| | starting | ending |
| Transcript name | position | position |
| T11811_T12 (SEQ. ID NO: 278) | 609 | 693 |
| T11811_T13 (SEQ. ID NO: 279) | 540 | 624 |
| T11811_T15 (SEQ. ID NO: 280) | 631 | 715 |
| T11811_T24 (SEQ. ID NO: 281) | 894 | 978 |
| T11811_T3 (SEQ. ID NO: 276) | 540 | 624 |
| T11811_T6 (SEQ. ID NO: 277) | 540 | 624 |

Segment cluster T11811_N18 (SEQ. ID NO:297) according to the present invention is supported by 61 libraries. The number of libraries was determined as previously described. This segment can be found in the following transcript(s): T11811_T12 (SEQ. ID NO:278), T11811_T13 (SEQ. ID NO:279), T11811_T15 (SEQ. ID NO:280), T11811_T24 (SEQ. ID NO:281), T11811_T3 (SEQ. ID NO:276) and TI 1811_T6 (SEQ. ID NO:277). Table 171 below describes the starting and ending position of this segment on each transcript.

TABLE 171
Segment location on transcripts

| | Segment | Segment |
|---|---|---|
| | starting | ending |
| Transcript name | position | position |
| T11811_T12 (SEQ. ID NO: 278) | 720 | 792 |
| T11811_T13 (SEQ. ID NO: 279) | 651 | 723 |
| T11811_T15 (SEQ. ID NO: 280) | 742 | 814 |
| T11811_T24 (SEQ. ID NO: 281) | 1005 | 1077 |
| T11811_T3 (SEQ. ID NO: 276) | 651 | 723 |
| T11811_T6 (SEQ. ID NO: 277) | 651 | 723 |

Segment cluster T11811_N20 (SEQ. ID NO:298) according to the present invention is supported by 7 libraries. The number of libraries was determined as previously described. This segment can be found in the following transcript(s): T11811_T6 (SEQ. ID NO:277). Table 172 below describes the starting and ending position of this segment on each transcript.

TABLE 172
Segment location on transcripts

| | Segment | Segment |
|---|---|---|
| | starting | ending |
| Transcript name | position | position |
| T11811_T6 (SEQ. ID NO: 277) | 724 | 818 |

Segment cluster T11811_N22 (SEQ. ID NO:300) according to the present invention is supported by 58 libraries. The number of libraries was determined as previously described. This segment can be found in the following transcript(s): T11811_T12 (SEQ. ID NO:278), T11811_T15 (SEQ. ID NO:280), T11811_T24 (SEQ. ID NO:281), T11811_T3 (SEQ. ID NO:276) and T11811_T6 (SEQ. ID NO:277). Table 173 below describes the starting and ending position of this segment on each transcript.

TABLE 173
Segment location on transcripts

| | Segment | Segment |
|---|---|---|
| | starting | ending |
| Transcript name | position | position |
| T11811_T12 (SEQ. ID NO: 278) | 809 | 841 |
| T11811_T15 (SEQ. ID NO: 280) | 831 | 863 |
| T11811_T24 (SEQ. ID NO: 281) | 1094 | 1126 |
| T11811_T3 (SEQ. ID NO: 276) | 740 | 772 |
| T11811_T6 (SEQ. ID NO: 277) | 835 | 867 |

Segment cluster T11811_N23 (SEQ. ID NO:301) according to the present invention is supported by 7 libraries. The number of libraries was determined as previously described. This segment can be found in the following transcript(s): T11811_T3 (SEQ. ID NO:276). Table 174 below describes the starting and ending position of this segment on each transcript.

TABLE 174
Segment location on transcripts

| | Segment | Segment |
|---|---|---|
| | starting | ending |
| Transcript name | position | position |
| T11811_T3 (SEQ. ID NO: 276) | 773 | 871 |

Expression of Homo sapiens myosin, light polypeptide 7, regulatory (MYL7) T11811 transcripts which are detectable by amplicon as depicted in sequence name T11811_seg14WT (SEQ. ID NO: 311) specifically in heart tissue

Expression of Homo sapiens myosin, light polypeptide 7, regulatory (MYL7) transcripts detectable by or according to seg14WT-T11811_seg14WT (SEQ. ID NO: 311) amplicon and primers T11811_seg14WTF (SEQ. ID NO: 309) and T11811_seg14WTR (SEQ. ID NO: 310) was measured by real time PCR. In parallel the expression of four housekeeping genes-SDHA (GenBank Accession No. NM_{—}004168 (SEQ. ID NO: 33); amplicon-SDHA-amplicon (SEQ. ID NO:36)), Ubiquitin (GenBank Accession No. BC000449 (SEQ. ID NO: 29); amplicon-Ubiquitin-amplicon (SEQ. ID NO: 32)), RPL19 (GenBank Accession No. NM_{—}000981 (SEQ. ID NO: 21); RPL19 amplicon (SEQ. ID NO: 24)) and TATA box (GenBank Accession No. NM_{—}003194 (SEQ. ID NO: 25); TATA amplicon (SEQ. ID NO: 28)) was measured similarly. For each RT sample, the expression of the above amplicon was normalized to the geometric mean of the quantities of the housekeeping genes. The normalized quantity of each RT sample was then divided by the average of the quantities of the heart samples (sample numbers 44, 45 and 46, Table 1_{—}6 above), to obtain a value of relative expression of each sample relative to average of the heart samples.

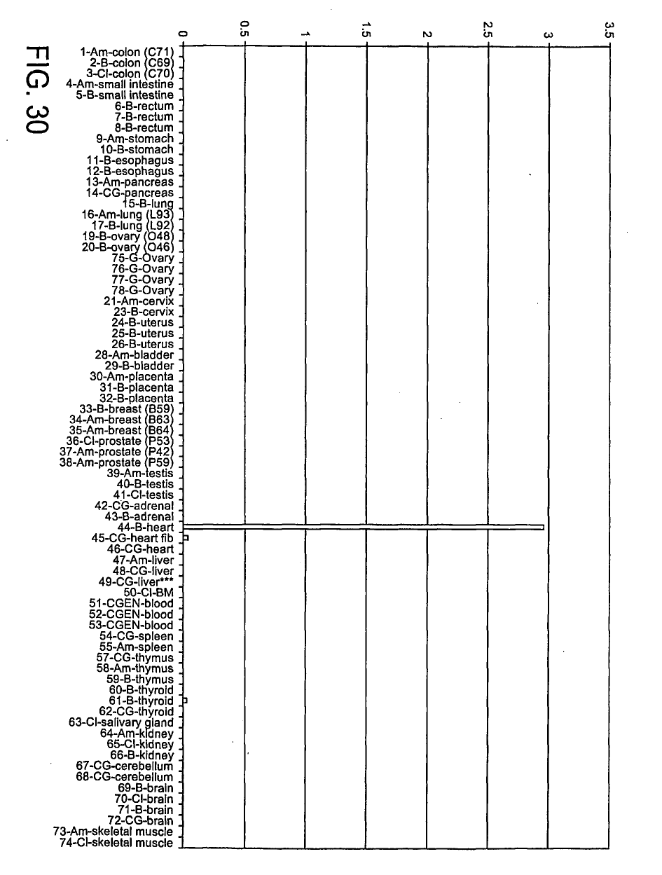

FIG. 30 is a histogram showing relative expression of the above-indicated Homo sapiens myosin, light polypeptide 7, regulatory (MYL7) transcripts in heart tissue samples as opposed to other tissues.

As is evident from FIG. 30, the expression of Homo sapiens myosin, light polypeptide 7, regulatory (MYL7) transcripts detectable by the above amplicon in 2 of the heart tissue samples was significantly higher than in the other samples (sample numbers 1, 2, 3, 4, 5, 6, 7, 8, 9, 10, 11, 12, 13, 14, 15, 16, 17, 19, 20, 75, 76, 77, 78, 21, 23, 24, 25, 26, 28, 29, 30, 31, 32, 33, 34, 35, 36, 37, 38, 39, 40, 41, 42, 43, 47, 48, 49, 50, 51, 52, 53, 54, 55, 57, 58, 59, 60, 61, 62, 63, 64, 65, 66, 67, 68, 69, 70, 71, 72, 73 and 74, Table 1_{—}6 above).

Primer pairs are also optionally and preferably encompassed within the present invention; for example, for the above experiment, the following primer pair was used as a non-limiting illustrative example only of a suitable primer pair: T11811_seg14WTF (SEQ. ID NO: 309) forward primer; and T11811_seg14WTR (SEQ. ID NO: 310) reverse primer.

The present invention also preferably encompasses any amplicon obtained through the use of any suitable primer pair; for example, for the above experiment, the following amplicon was obtained as a non-limiting illustrative example only of a suitable amplicon: T11811_seg14WT (SEQ. ID NO: 311).

Forward Primer (T11811_seg14WTF
(SEQ. ID NO: 309)): GGAAGGTGAGTGTCCCAGAGG
Reverse Primer (T11811_seg14WTR
(SEQ. ID NO: 310)): GTGAGGAAGACGGTGAAGTTGAT
Amplicon (T11811_seg14WT
(SEQ. ID NO: 311)): GGAAGGTGAGTGTCCCAGAGGAGGAGCTGG
ACGCCATGCTGCAAGAGGGCAAGGGCCCCATCAACTTCACCGTCTTCC
TCAC

Expression of Homo sapiens myosin, light polypeptide 7, regulatory (MYL7) T11811 transcripts which are detectable by amplicon as depicted in sequence name T11811_seg7-8-9 (SEQ. ID NO: 314) specifically in heart tissue

Expression of Homo sapiens myosin, light polypeptide 7, regulatory (MYL7) transcripts detectable by or according to seg7-8-9-T11811_seg7-8-9 (SEQ. ID NO: 314) amplicon and primers T11811_seg7-8-9F (SEQ. ID NO: 312) and T11811seg7-8-9R (SEQ. ID NO: 313) was measured by real time PCR. In parallel the expression of four housekeeping genes-SDHA (GenBank Accession No. NM_{—}004168 (SEQ. ID NO: 33); amplicon-SDHA-amplicon (SEQ. ID NO:36)), Ubiquitin (GenBank Accession No. BC000449 (SEQ. ID NO: 29); amplicon-Ubiquitin-amplicon (SEQ. ID NO: 32)), RPL19 (GenBank Accession No. NM_{—}000981 (SEQ. ID NO: 21); RPL19 amplicon (SEQ. ID NO: 24)) and TATA box (GenBank Accession No. NM_{—}003194 (SEQ. ID NO: 25); TATA amplicon (SEQ. ID NO: 28)) was measured similarly. For each RT sample, the expression of the above amplicon was normalized to the geometric mean of the quantities of the housekeeping genes. The normalized quantity of each RT sample was then divided by the avarage of the quantities of the heart samples (sample numbers 44 and 45, Table 1_{—}6 above), to obtain a value of relative expression of each sample relative to avarage of the heart samples.

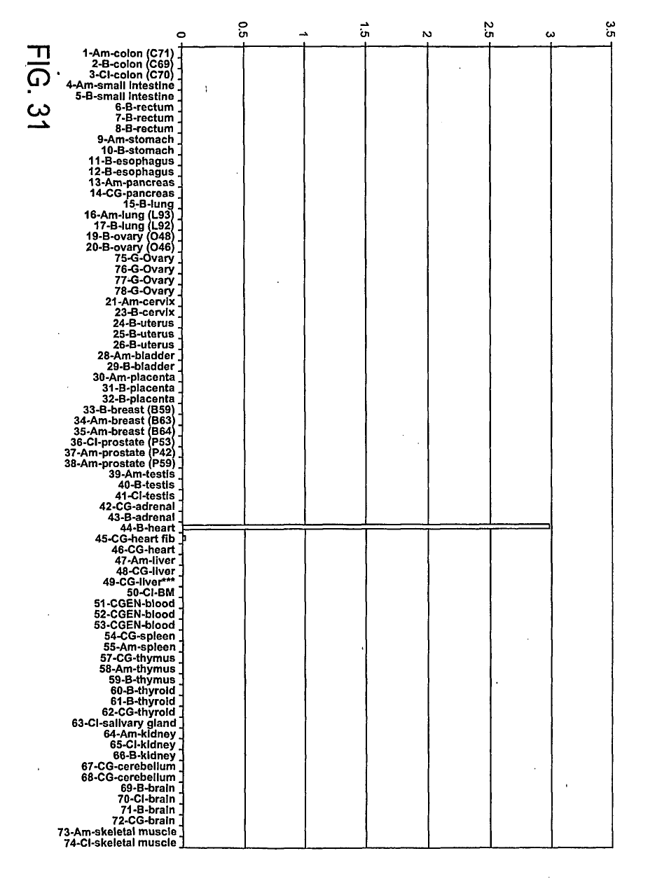

FIG. 31 is a histogram showing relative expression of the above-indicated Homo sapiens myosin, light polypeptide 7, regulatory (MYL7) transcripts in heart tissue samples as opposed to other tissues.

As is evident from FIG. 31, the expression of Homo sapiens myosin, light polypeptide 7, regulatory (MYL7) transcripts detectable by the above amplicon in 2 of the heart tissue samples was significantly higher than in the other samples (sample numbers 1, 2, 3, 4, 5, 6, 7, 8, 9, 10, 11, 12, 13, 14, 15, 16, 17, 19, 20, 75, 76, 77, 78, 21, 23, 24, 25, 26, 28, 29, 30, 31, 32, 33, 34, 35, 36, 37, 38, 39, 40, 41, 42, 43, 47, 48, 49, 50, 51, 52, 53, 54, 55, 57, 58, 59, 60, 61, 62, 63, 64, 65, 66, 67, 68, 69, 70, 71, 72, 73 and 74, Table 1_{—}6 above).

Primer pairs are also optionally and preferably encompassed within the present invention; for example, for the above experiment, the following primer pair was used as a non-limiting illustrative example only of a suitable primer pair: T11811_seg7-8-9F (SEQ. ID NO: 312) forward primer; and T11811_seg7-8-9R (SEQ. ID NO: 313) reverse primer.

The present invention also preferably encompasses any amplicon obtained through the use of any suitable primer pair; for example, for the above experiment, the following amplicon was obtained as a non-limiting illustrative example only of a suitable amplicon: T11811_seg7-8-9 (SEQ. ID NO: 314).

Forward Primer (T11811_seg7-8-9F
(SEQ. ID NO: 312)): CCCACCTTCCCTAGAGCTGG
Reverse Primer (T11811_seg7-8-9R
(SEQ. ID NO: 313)): AGGTCTGCCTTGCAGATGATG
Amplicon (T11811_seg7-8-9
(SEQ. ID NO: 314)): CCCACCTTCCCTAGAGCTGGGGGCTGCTCC
CACCTGAAGGCCCCCATCCCACAGGCCTTCAGCTGTATCGACCAGAATCG
TGATGGCATCATCTGCAAGGCAGACCT

Expression of Homo sapiens myosin, light polypeptide 7, regulatory (MYL7) T11811 transcripts which are detectable by amplicon as depicted in sequence name T11811_seg23 (SEQ. ID NO: 317) specifically in heart tissue

Expression of Homo sapiens myosin, light polypeptide 7, regulatory (MYL7) transcripts detectable by or according to seg23-T11811_seg23_F2R2 (SEQ. ID NO: 317) amplicon and primers T11811_seg23F2 (SEQ. ID NO: 315) and T11811_seg23R2 (SEQ. ID NO: 316) was measured by real time PCR. In parallel the expression of four housekeeping genes-SDHA (GenBank Accession No. NM_{—}004168 (SEQ. ID NO: 33); amplicon-SDHA-amplicon (SEQ. ID NO:36)), Ubiquitin (GenBank Accession No. BC000449 (SEQ. ID NO: 29); amplicon-Ubiquitin-amplicon (SEQ. ID NO: 32)), RPL19 (GenBank Accession No. NM_{—}000981 (SEQ. ID NO: 21); RPL19 amplicon (SEQ. ID NO: 24)) and TATA box (GenBank Accession No. NM_{—}003194 (SEQ. ID NO: 25); TATA amplicon (SEQ. ID NO: 28)) was measured similarly. For each RT sample, the expression of the above amplicon was normalized to the geometric mean of the quantities of the housekeeping genes. The normalized quantity of each RT sample was then divided by the avarage of the quantities of the heart samples (sample numbers 44, 45 and 46, Table 1_{—}6 above), to obtain a value of relative expression of each sample relative to avarage of the heart samples.

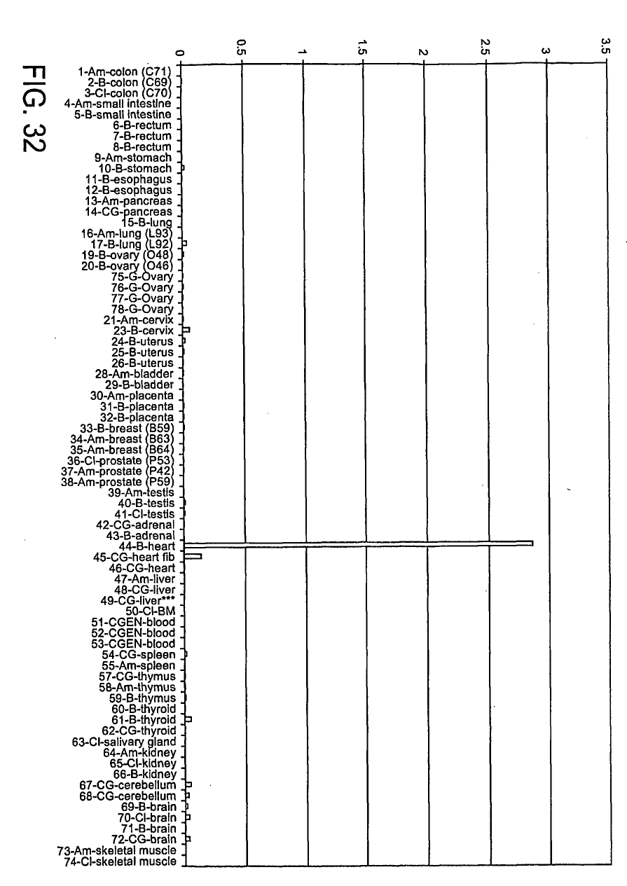

FIG. 32 is a histogram showing relative expression of the above-indicated Homo sapiens myosin, light polypeptide 7, regulatory (MYL7) transcripts in heart tissue samples as opposed to other tissues.

As is evident from FIG. 32, the expression of Homo sapiens myosin, light polypeptide 7, regulatory (MYL7) transcripts detectable by the above amplicon in 2 of the heart tissue samples was significantly higher than in the other samples (sample numbers 1, 2, 3, 4, 5, 6, 7, 8, 9, 10, 11, 12, 13, 14, 15, 16, 17, 19, 20, 75, 76, 77, 78, 21, 23, 24, 25, 26, 28, 29, 30, 31, 32, 33, 34, 35, 36, 37, 38, 39, 40, 41, 42, 43, 47, 48, 49, 50, 51, 52, 53, 54, 55, 57, 58, 59, 60, 61, 62, 63, 64, 65, 66, 67, 68, 69, 70, 71, 72, 73 and 74, Table 1_{—}6 above).

Primer pairs are also optionally and preferably encompassed within the present invention; for example, for the above experiment, the following primer pair was used as a non-limiting illustrative example only of a suitable primer pair: T11811_seg23F2 (SEQ. ID NO: 315) forward primer; and T11811_seg23R2 (SEQ. ID NO: 316) reverse primer.

The present invention also preferably encompasses any amplicon obtained through the use of any suitable primer pair; for example, for the above experiment, the following amplicon was obtained as a non-limiting illustrative example only of a suitable amplicon: T11811_seg23F2R2 (SEQ. ID NO: 317).

Forward Primer (T11811_seg23F2
(SEQ. ID NO: 315)): AGGCAGACAAGTTCTCTCCAGCT
Reverse Primer (T11811_seg23R2
(SEQ. ID NO: 316)): GGTGAAGGCCCAGAGAAGG
Amplicon (T11811_seg23_F2R2
(SEQ. ID NO: 317)): AGGCAGACAAGTTCTCTCCAGCTGAGGTGA
GGCTGCCCAGCCCCTTCAATACTCATCCCCAGCACCTTCTCTGGGCC
TTCACC

It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-61. (canceled)

62. An isolated polynucleotide comprising a nucleic acid sequence set forth in a member selected from the group consisting of SEQ ID NOs:37-39 and homologues and fragments thereof.

63. The polynucleotide of claim 62, wherein the nucleic acid sequence encodes a polypeptide having an amino acid sequence set forth in a member selected from the group consisting of SEQ ID NOs:57-58.

64. The polynucleotide of claim 62, wherein the nucleic acid sequence encodes a polypeptide comprising contiguous amino acids having at least about 70%, 80%, 85%, 90%, 95% or 100% homology to any one of SEQ ID NO:326-328.

65. An isolated polynucleotide, comprising a nucleic acid sequence complementary to any one of the nucleic acid sequences of claim 62.

66. An isolated polynucleotide, comprising a nucleic acid sequence that hybridizes under stringent conditions to any one of the nucleic acid sequences of claim 62.

67. An isolated polypeptide having an amino acid sequence encoded by any one of the nucleic acid sequence of claim 62.

68. An isolated polypeptide comprising an amino acid sequence at least about 70%, 80%, 85%, 90% or 95% homologous to SEQ ID NOs:57-58.

69. The isolated polypeptide of claim 68, having an amino acid sequence as set forth in any one of SEQ ID NOs:57-58.

70. The polypeptide of claim 68, comprising a first portion having an amino acid sequence being at least about 90% homologous to amino acids 1-43 of SEQ ID NO:57, and a second portion having amino acid sequence being at least about 70%, 80%, 85%, 90%, 95% or 100% homologous to SEQ ID NO:326, wherein the first amino acid sequence and the second amino acid sequence are contiguous and in a sequential order.

71. The polypeptide of claim 68, comprising a first portion having an amino acid sequence being at least about 90% homologous to amino acids 1-23 of SEQ ID NO:58, and a second portion having amino acid sequence being at least about 70%, 80%, 85%, 90%, 95% or 100% homologous to SEQ ID NO:327, wherein the first amino acid sequence and the second amino acid sequence are contiguous and in a sequential order.

72. A polypeptide comprising an amino acid sequence at least about 70%, 80%, 85%, 90%, 95% or 100% homologous to any one of SEQ ID NO:326-SEQ ID NO:328.

73. An antibody which binds to at least one epitope of a polypeptide having an amino acid sequence according to claim 68.

74. The antibody of claim 73, wherein said antibody is capable of differentiating between a polypeptide having the epitope and a corresponding known protein.

75. An expression vector comprising any one of the polynucleotide sequence according to claim 62.

76. A host cell comprising the vector according to claim 75.

77. A process for producing a polypeptide comprising: culturing the host cell according to claim 76 under conditions suitable to produce the polypeptide encoded by the polynucleotide; and recovering said polypeptide.

78. An isolated polynucleotide segment, consisting of a nucleic acid sequence selected from the group consisting of: SEQ ID NOs:40-53.

79. An isolated polynucleotide segment consisting of a nucleic acid sequence complementary to any one of the nucleic acid sequences of the segments of claim 78.

80. An isolated polynucleotide segment consisting of a nucleic acid sequence that hybridizes under stringent conditions to any one of the nucleic acid sequences of the segments of claim 78.

81. A kit for detecting cancer, comprising a marker capable of detecting a DLL3 protein or a variant thereof or a polynucleotide encoding same selected from the group consisting of:
a polypeptide comprising an amino acid sequence at least about 70%, 80%, 85%, 90%, 95% or 100% homologous to any one of SEQ ID NOs:57-58; or a polypeptide comprising an amino acid sequence at least about 70%, 80%, 85%, 90%, 95% or 100% homologous to any on of SEQ ID NO:326-328; or a polypeptide having a sequence as set forth in any one of SEQ ID NOs:54-56, 59; or
a polynucleotide comprising a nucleic acid sequence set forth in a member selected from the group consisting of SEQ ID NOs:37-39 and homologues and fragments thereof; or
a polynucleotide comprising a nucleic acid sequence set forth in a member selected from the group consisting of SEQ ID NOs:40-53, 62, 67, 70, 73; or
a polynucleotide having a sequence as set forth in any one of SEQ ID NOs:74-75.

82. The kit of claim 81, wherein said kit comprises an antibody which binds to at least one epitope of a polypeptide having an amino acid sequence at least about 70%, 80%, 85%, 90%, or 95% homologous to SEQ ID NOs:57-58, and wherein said kit further comprises at least one reagent for performing an immunoassay.

83. The kit of claim 82, wherein said immunoassay is selected from the group consisting of an ELISA, a RIA (radio immunoassay), a slot blot, immunohistochemical assay, FACS (fluorescence activated cell sorting), a radio-imaging assay or a Western blot.

84. The kit of claim 81, wherein the cancer is lung cancer.

85. The kit of claim 81, wherein the cancer is invasive or metastatic.

86. The kit of claim 81, wherein said kit comprises at least one oligonucleotide, probe or primer pair.

87. The kit of claim 86, wherein said kit comprises at least one oligonucleotide capable of selectively hybridizing to a nucleic acid sequence as set forth in any one of SEQ ID NOs:37-53, 74-75, or a homologue or fragment thereof.

88. The kit of claim 86, wherein said at least one primer pair amplifies an amplicon comprising the sequence as set forth in SEQ ID NO:62, 67, 70, 73.

89. The primer pair of claim 88, comprising a pair of isolated oligonucleotides selected from the group consisting of SEQ ID NO:65 and SEQ ID NO:66; SEQ ID NO:66 and SEQ ID NO:69; SEQ ID NO:69 and SEQ ID NO:72; SEQ ID NO:60 and SEQ ID NO:61.

90. The kit of claim 86, wherein said kit comprises at least one oligonucleotide or probe capable of selectively hybridizing to a polynucleotide comprising a nucleic acid sequence set forth in a member selected from the group consisting of SEQ ID NOs:37-39 and homologues and fragments diagnosis.

91. The kit of claim 90, wherein the probe has a nucleic acid sequence selected from the group consisting of SEQ ID NOs:64, 68 and 71.

92. A method for at least one of detecting cancer, monitoring cancer progression, monitoring cancer-treatment efficacy, detecting acute or chronic exacerbation of cancer and selecting a therapy for cancer, comprising detecting in a sample differential expression of at least one polypeptide of a DLL3 protein or a variant thereof selected from the group consisting of:
a polypeptide comprising an amino acid sequence at least about 70%, 80%, 85%, 90%, 95% or 100% homologous to any one of SEQ ID NOs:57-58; or
a polypeptide comprising an amino acid sequence at least about 70%, 80%, 85%, 90%, 95% or 100% homologous to any on of SEQ ID NO:326-328; or
a polypeptide having a sequence as set forth in any one of SEQ ID NOs: 54-56, and 59.

93. The method of claim 92, wherein the cancer is lung cancer.

94. The method of claim 92, wherein said cancer is invasive or metastatic.

95. The method of claim 92, wherein detecting the differential expression of the polypeptide is performed with an antibody that binds to at least one epitope of a polypeptide having an amino acid sequence at least about 70%, 80%, 85%, 90%, or 95% homologous to SEQ ID NOs:57-58,

96. A method for at least one of detecting cancer, monitoring cancer progression, monitoring cancer-treatment efficacy, detecting acute or chronic exacerbation of cancer and selecting a therapy for cancer, comprising detecting in a sample differential expression of at least one polynucleotide or a part thereof, encoding a DLL3 protein or a variant thereof, selected from the group consisting of:
a polynucleotide comprising a nucleic acid sequence set forth in a member selected from the group consisting of SEQ ID NOs:37-39 and homologues and fragments thereof; or
polynucleotide comprising a nucleic acid sequence set forth in a member selected from the group consisting of SEQ ID NOs: 40-53, 62, 67, 70, 73; or
a polynucleotide having a sequence as set forth in any one of SEQ ID NOs: 74-75.

97. The method of claim 96, wherein the cancer is lung cancer.

98. The method of claim 96, wherein said cancer is invasive or metastatic.

99. The method of claim 96, wherein detecting the differential expression of the at least one polynucleotide is performed using a pair of isolated oligonucleotides selected from the group consisting of SEQ ID NO:65 and SEQ ID NO:66; SEQ ID NO:66 and SEQ ID NO:69; SEQ ID NO:69 and SEQ ID NO:72; SEQ ID NO:60 and SEQ ID NO:61.

100. The method of claim 96, wherein detecting the differential expression of the at least one polynucleotide is performed using a probe having a nucleic acid sequence selected from the group consisting of SEQ ID NOs:64, 68 and 71.


SEQUENCE LISTING (398 sequences)

SEQ ID NO: 1 - DNA, 1536 bp, Homo sapiens
gtgacgcgaggctctgcggagaccaggagtcagactgtaggacgacctcgggtcccacgtgtccccggtactcgccggccggagcccccggcttcccggggccgggggaccttagcggcacccacacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctggcaacagctggcctgcagcgcatgggctggcacaaccgggtggggcagatcctgcaccctgaggaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggtaaaaaaaaaaaaaaaaaaaaaaaaaaaaaaaaaaa

SEQ ID NO: 2 - DNA, 19 bp, Artificial Sequence
  Synthetic oligonucleotide
tgagagtgattcgcgtggg

SEQ ID NO: 3 - DNA, 21 bp, Artificial Sequence
  Synthetic oligonucleotide
ccagggtacgaggctttcaat

SEQ ID NO: 4 - DNA, 91 bp, Artificial Sequence
  Synthetic oligonucleotide
tgagagtgattcgcgtgggtacccgcaagagccagcttgctcgcatacagacggacagtgtggtggcaacattgaaagcctcgtaccctgg

SEQ ID NO: 5 - DNA, 1261 bp, Homo sapiens
ccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaattt

SEQ ID NO: 6 - DNA, 21 bp, Artificial Sequence
  Synthetic oligonucleotide
tgacactggcaaaacaatgca

SEQ ID NO: 7 - DNA, 21 bp, Artificial Sequence
  Synthetic oligonucleotide
ggtccttttcaccagcaagct

SEQ ID NO: 8 - DNA, 94 bp, Artificial Sequence
  Synthetic oligonucleotide
tgacactggcaaaacaatgcagactttgctttccttggtcaggcagtataatccaaagatggtcaaggtcgcaagcttgctggtgaaaaggacc

SEQ ID NO: 9 - DNA, 1261 bp, Homo sapiens
cttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccaaaaaaaaaaaaaaa

SEQ ID NO: 10 - DNA, 20 bp, Artificial Sequence
  Synthetic oligonucleotide
tgcaccaccaactgcttagc

SEQ ID NO: 11 - DNA, 19 bp, Artificial Sequence
  Synthetic oligonucleotide
ccatcacgccacagtttcc

SEQ ID NO: 12 - DNA, 116 bp, Artificial Sequence
  Synthetic oligonucleotide
tgcaccaccaactgcttagcacccctggccaaggtcatccatgacaactttggtatcgtggaaggactcatgaccacagtccatgccatcactgccacccagaagactgtggatgg

SEQ ID NO: 13 - DNA, 2650 bp, Homo sapiens
agggacagcccagaggaggcgtggccacgctgccggcggaagtggagccctccgcgagcgcgcgaggccgccggggcaggcggggaaaccggacagtaggggcggggccgggccggcgatggggatgcgggagcactacgcggagctgcacccgtgcccgccggaattggggatgcagagcagcggcagcgggtatggcaggcagccggcgggccggcctccagcgcaggtgcccgagaggcaggggctggcctgggatgcgcgcgcacctgccctcgccccgccccgcccgcacgaggggtggtggccgaggccccgccccgcacgcctcgcctgaggcgggtccgctcagcccaggcgcccgcccccgcccccgccgattaaatgggccggcggggctcagcccccggaaacggtcgtaacttcggggctgcgagcgcggagggcgacgacgacgaagcgcagacagcgtcatggcagagcaggtggccctgagccggacccaggtgtgcgggatcctgcgggaagagcttttccagggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatggatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaaccacctactgcagatgctgtgtctggtggccatggagaagcccgcctccaccaactcagatgacgtccgtgatgagaaggtcaaggtgttgaaatgcatctcagaggtgcaggccaacaatgtggtcctgggccagtacgtggggaaccccgatggagagggcgaggccaccaaagggtacctggacgaccccacggtgccccgcgggtccaccaccgccacttttgcagccgtcgtcctctatgtggagaatgagaggtgggatggggtgcccttcatcctgcgctgcggcaaggccctgaacgagcgcaaggccgaggtgaggctgcagttccatgatgtggccggcgacatcttccaccagcagtgcaagcgcaacgagctggtgatccgcgtgcagcccaacgaggccgtgtacaccaagatgatgaccaagaagccgggcatgttcttcaaccccgaggagtcggagctggacctgacctacggcaacagatacaagaacgtgaagctccctgacgcctacgagcgcctcatcctggacgtcttctgcgggagccagatgcacttcgtgcgcagcgacgagctccgtgaggcctggcgtattttcaccccactgctgcaccagattgagctggagaagcccaagcccatcccctatatttatggcagccgaggccccacggaggcagacgagctgatgaagagagtgggtttccagtatgagggcacctacaagtgggtgaacccccacaagctctgagccctgggcacccacctccacccccgccacggccaccctccttcccgccgcccgaccccgagtcgggaggactccgggaccattgacctcagctgcacattcctggccccgggctctggccaccctggcccgcccctcgctgctgctactacccgagcccagctacattcctcagctgccaagcactcgagaccatcctggcccctccagaccctgcctgagcccaggagctgagtcacctcctccactcactccagcccaacagaaggaaggaggagggcgcccattcgtctgtcccagagcttattggccactgggtctcactcctgagtggggccagggtgggagggagggacaagggggaggaaaggggcgagcacccacgtgagagaatctgcctgtggccttgcccgccagcctcagtgccacttgacattccttgtcaccagcaacatctcgagccccctggatgtcccctgtcccaccaactctgcactccatggccaccccgtgccacccgtaggcagcctctctgctataagaaaagcagacgcagcagctgggacccctcccaacctcaatgccctgccattaaatccgcaaacagccaaaaaaaaaaaaaaaaaaaa

SEQ ID NO: 14 - DNA, 20 bp, Artificial Sequence
  Synthetic oligonucleotide
gaggccgtcaccaagaacat

SEQ ID NO: 15 - DNA, 19 bp, Artificial Sequence
  Synthetic oligonucleotide
ggacagccggtcagagctc

SEQ ID NO: 16 - DNA, 111 bp, Artificial Sequence
  Synthetic oligonucleotide
gaggccgtcaccaagaacattcacgagtcctgcatgagccagataggctggaaccgcatcatcgtggagaagcccttcgggagggacctgcagagctctgaccggctgtcc

SEQ ID NO: 17 - DNA, 541 bp, Homo sapiens
cttttcgatccgccatctgcggtggagccgccaccaaaatgcagattttcgtgaaaacccttacggggaagaccatcaccctcgaggttgaaccctcggatacgatagaaaatgtaaaggccaagatccaggataaggaaggaattcctcctgatcagcagagactgatctttgctggcaagcagctggaagatggacgtactttgtctgactacaatattcaaaaggagtctactcttcatcttgtgttgagacttcgtggtggtgctaagaaaaggaagaagaagtcttacaccactcccaagaagaataagcacaagagaaagaaggttaagctggctgtcctgaaatattataaggtggatgagaatggcaaaattagtcgccttcgtcgagagtgcccttctgatgaatgtggtgctggggtgtttatggcaagtcactttgacagacattattgtggcaaatgttgtctgacttactgtttcaacaaaccagaagacaagtaactgtatgagttaataaaagacatgaactaaca

SEQ ID NO: 18 - DNA, 20 bp, Artificial Sequence
  Synthetic oligonucleotide
ctggcaagcagctggaagat

SEQ ID NO: 19 - DNA, 23 bp, Artificial Sequence
  Synthetic oligonucleotide
tttcttagcaccaccacgaagtc

SEQ ID NO: 20 - DNA, 101 bp, Artificial Sequence
  Synthetic oligonucleotide
ctggcaagcagctggaagatggacgtactttgtctgactacaatattcaaaaggagtctactcttcatcttgtgttgagacttcgtggtggtgctaagaaa

SEQ ID NO: 21 - DNA, 748 bp, Homo sapiens
gcagataatgggaggagccgggcccgagcgagctctttcctttcgctgctgcggccgcagccatgagtatgctcaggcttcagaagaggctcgcctctagtgtcctccgctgtggcaagaagaaggtctggttagaccccaatgagaccaatgaaatcgccaatgccaactcccgtcagcagatccggaagctcatcaaagatgggctgatcatccgcaagcctgtgacggtccattcccgggctcgatgccggaaaaacaccttggcccgccggaagggcaggcacatgggcataggtaagcggaagggtacagccaatgcccgaatgccagagaaggtcacatggatgaggagaatgaggattttgcgccggctgctcagaagataccgtgaatctaagaagatcgatcgccacatgtatcacagcctgtacctgaaggtgaaggggaatgtgttcaaaaacaagcggattctcatggaacacatccacaagctgaaggcagacaaggcccgcaagaagctcctggctgaccaggctgaggcccgcaggtctaagaccaaggaagcacgcaagcgccgtgaagagcgcctccaggccaagaaggaggagatcatcaagactttatccaaggaggaagagaccaagaaataaaacctcccactttgtctgtacatactggcctctgtgattacatagatcagccattaaaataaaacaagccttaatctgcaaaaaaaaaaaaaaaa

SEQ ID NO: 22 - DNA, 23 bp, Artificial Sequence
  Synthetic oligonucleotide
tggcaagaagaaggtctggttag

SEQ ID NO: 23 - DNA, 22 bp, Artificial Sequence
  Synthetic oligonucleotide
tgatcagcccatctttgatgag

SEQ ID NO: 24 - DNA, 101 bp, Artificial Sequence
  Synthetic oligonucleotide
tggcaagaagaaggtctggttagaccccaatgagaccaatgaaatcgccaatgccaactcccgtcagcagatccggaagctcatcaaagatgggctgatca

SEQ ID NO: 25 - DNA, 1867 bp, Homo sapiens
ggttcgctgtggcgggcgcctgggccgccggctgtttaacttcgcttccgctggcccatagtgatctttgcagtgacccagcagcatcactgtttcttggcgtgtgaagataacccaaggaattgaggaagttgctgagaagagtgtgctggagatgctctaggaaaaaattgaatagtgagacgagttccagcgcaagggtttctggtttgccaagaagaaagtgaacatcatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttaaaaaaaaaaaaaaaaaaaaaa

SEQ ID NO: 26 - DNA, 20 bp, Artificial Sequence
  Synthetic oligonucleotide
cggtttgctgcggtaatcat

SEQ ID NO: 27 - DNA, 21 bp, Artificial Sequence
  Synthetic oligonucleotide
tttcttgctgccagtctggac

SEQ ID NO: 28 - DNA, 122 bp, Artificial Sequence
  Synthetic oligonucleotide
cggtttgctgcggtaatcatgaggataagagagccacgaaccacggcactgattttcagttctgggaaaatggtgtgcacaggagccaagagtgaagaacagtccagactggcagcaagaaa

SEQ ID NO: 29 - DNA, 2201 bp, Homo sapiens
cgggatttgggtcgcggttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtggagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgatcagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaaaaaaaaaaaaaaaaaaaaaaaa

SEQ ID NO: 30 - DNA, 19 bp, Artificial Sequence
  Synthetic oligonucleotide
atttgggtcgcggttcttg

SEQ ID NO: 31 - DNA, 21 bp, Artificial Sequence
  Synthetic oligonucleotide
tgccttgacattctcgatggt

SEQ ID NO: 32 - DNA, 133 bp, Artificial Sequence
  Synthetic oligonucleotide
atttgggtcgcggttcttgtttgtggatcgctgtgatcgtcacttgacaatgcagatcttcgtgaagactctgactggtaagaccatcaccctcgaggttgagcccagtgacaccatcgagaatgtcaaggca

SEQ ID NO: 33 - DNA, 2207 bp, Homo sapiens
tggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcgcagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggacggtctctgcgatatgataccagctattttgtggagtattttgccttggatctcctgatggagaacggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccccacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtcgatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacagacctggtggagaccctggagctgcagaacctgatgctgtgtgcgctgcagaccatctacggagcagaggcgcggaaggagtcacggggcgcgcatgccagggaagactacaaggtgcggattgatgagtacgattactccaagcccatccaggggcaacagaagaagccctttgaggagcactggaggaagcacaccctgtcctttgtggacgttggcactgggaaggtcactctggaatatagacccgtaatcgacaaaactttgaacgaggctgactgtgccaccatcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgag

SEQ ID NO: 34 - DNA, 20 bp, Artificial Sequence
  Synthetic oligonucleotide
tgggaacaagagggcatctg

SEQ ID NO: 35 - DNA, 22 bp, Artificial Sequence
  Synthetic oligonucleotide
ccaccactgcatcaaattcatg

SEQ ID NO: 36 - DNA, 86 bp, Artificial Sequence
  Synthetic oligonucleotide
tgggaacaagagggcatctgctaaagtttcagattccatttctgctcagtatccagtagtggatcatgaatttgatgcagtggtgg

SEQ ID NO: 37 - DNA, 2017 bp, Homo sapiens
ggccattctctcagatataaggcttggaagccagcagctgcgactcccgagacccccccaccagaaggccatggtctccccacggatgtccgggctcctctcccagactgtgatcctagcgctcattttcctcccccagacacggcccgctggcgtcttcgagctgcagatccactctttcgggccgggtccaggcccggctcccctgccgcctcttcttcagagtctgcctgaagcctgggctctcagaggaggccgccgagtccccgtgcgccctgggcgcggcgctgagtgcgcgcggaccggtctacaccgagcagcccggagcgcccgcgcctgatctcccactgcccgacggcctcttgcaggtgcccttccgggacgcctggcctggcaccttctctttcatcatcgaaacctggagagaggagttaggagaccagattggagggcccgcctggagcctgctggcgcgcgtggctggcaggcggcgcttggcagccggaggcccgtgggcccgggacattcagcgcgcaggcgcctgggagctgcgcttctcgtaccgcgcgcgctgcgagccgcctgccgtcgggaccgcgtgcacgcgcctctgccgtccgcgcagcgccccctcgcggtgcggtccgggactgcgcccctgcgcaccgctcgaggacgaatgtgaggcgccgctggtgtgccgagcaggctgcagccctgagcatggcttctgtgaacagcccggtgaatgccgatgcctagagggctggactggacccctctgcacggtccctgtctccaccagcagctgcctcagccccaggggcccgtcctctgctaccaccggatgccttgtccctgggcctgggccctgtgacgggaacccgtgtgccaatggaggcagctgtagtgagacacccaggtcctttgaatgcacctgcccgcgtgggttctacgggctgcggtgtgaggtgagcggggtgacatgtgcagatggaccctgcttcaacggcggcttgtgtgtcgggggtgcagaccctgactctgcctacatctgccactgcccacccggtttccaaggctccaactgtgagaagagggtggaccggtgcagcctgcagccatgccgcaatggcggactctgcctggacctgggccacgccctgcgctgccgctgccgcgccggcttcgcgggtcctcgctgcgagcacgacctggacgactgcgcgggccgcgcctgcgctaacggcggcacgtgtgtggagggcggcggcgcgcaccgctgctcctgcgcgctgggcttcggcggccgcgactgccgcgagcgcgcggacccgtgcgccgcgcgcccctgtgctcacggcggccgctgctacgcccacttctccggcctcgtctgcgcttgcgctcccggctacatgggagcgcggtgtgagttcccagtgcaccccgacggcgcaagcgccttgcccgcggccccgccgggcctcaggcccggggaccctcagcgctaccttttgcctccggctctgggactgctcgtggccgcgggcgtggccggcgctgcgctcttgctggtccacgtgcgccgccgtggccactcccaggatgctgggtctcgcttgctggctgggaccccggagccgtcagtccacgcactcccggatgcactcaacaacctaaggacgcaggagggttccggggatggtccgagctcgtccgtagattggaatcgccctgaagatgtagaccctcaagggatttatgtcatatctgctccttccatctacgctcgggaggcctgacgcgtctcctccatccgcacctggagtcagagcgtggatttttgtatttgctcggtggtgcccagtctctgccccagaggctttggagttcaatcttgaaggggtgtctgggggaactttactgttgcaagttgtaaataatggttatttatatcctattttttctcaccccatctctctagaaacacctataaaggctattattgtgatca

SEQ ID NO: 38 - DNA, 2057 bp, Homo sapiens
ggccattctctcagatataaggcttggaagccagcagctgcgactcccgagacccccccaccagaaggccatggtctccccacggatgtccgggctcctctcccagactgtgatcctagcgctcattttcctcccccaggtcagagccagacacggcccgctggcgtcttcgagctgcagatccactctttcgggccgggtccaggccctggggccccgcggtccccctgcagcgcccggctcccctgccgcctcttcttcagagtctgcctgaagcctgggctctcagaggaggccgccgagtccccgtgcgccctgggcgcggcgctgagtgcgcgcggaccggtctacaccgagcagcccggagcgcccgcgcctgatctcccactgcccgacggcctcttgcaggtgcccttccgggacgcctggcctggcaccttctctttcatcatcgaaacctggagagaggagttaggagaccagattggagggcccgcctggagcctgctggcgcgcgtggctggcaggcggcgcttggcagccggaggcccgtgggcccgggacattcagcgcgcaggcgcctgggagctgcgcttctcgtaccgcgcgcgctgcgagccgcctgccgtcgggaccgcgtgcacgcgcctctgccgtccgcgcagcgccccctcgcggtgcggtccgggactgcgcccctgcgcaccgctcgaggacgaatgtgaggcgccgctggtgtgccgagcaggctgcagccctgagcatggcttctgtgaacagcccggtgaatgccgatgcctagagggctggactggacccctctgcacggtccctgtctccaccagcagctgcctcagccccaggggcccgtcctctgctaccaccggatgccttgtccctgggcctgggccctgtgacgggaacccgtgtgccaatggaggcagctgtagtgagacacccaggtcctttgaatgcacctgcccgcgtgggttctacgggctgcggtgtgaggtgagcggggtgacatgtgcagatggaccctgcttcaacggcggcttgtgtgtcgggggtgcagaccctgactctgcctacatctgccactgcccacccggtttccaaggctccaactgtgagaagagggtggaccggtgcagcctgcagccatgccgcaatggcggactctgcctggacctgggccacgccctgcgctgccgctgccgcgccggcttcgcgggtcctcgctgcgagcacgacctggacgactgcgcgggccgcgcctgcgctaacggcggcacgtgtgtggagggcggcggcgcgcaccgctgctcctgcgcgctgggcttcggcggccgcgactgccgcgagcgcgcggacccgtgcgccgcgcgcccctgtgctcacggcggccgctgctacgcccacttctccggcctcgtctgcgcttgcgctcccggctacatgggagcgcggtgtgagttcccagtgcaccccgacggcgcaagcgccttgcccgcggccccgccgggcctcaggcccggggaccctcagcgctaccttttgcctccggctctgggactgctcgtggccgcgggcgtggccggcgctgcgctcttgctggtccacgtgcgccgccgtggccactcccaggatgctgggtctcgcttgctggctgggaccccggagccgtcagtccacgcactcccggatgcactcaacaacctaaggacgcaggagggttccggggatggtccgagctcgtccgtagattggaatcgccctgaagatgtagaccctcaagggatttatgtcatatctgctccttccatctacgctcgggaggcctgacgcgtctcctccatccgcacctggagtcagagcgtggatttttgtatttgctcggtggtgcccagtctctgccccagaggctttggagttcaatcttgaaggggtgtctgggggaactttactgttgcaagttgtaaataatggttatttatatcctattttttctcaccccatctctctagaaacacctataaaggctattattgtgatca

SEQ ID NO: 39 - DNA, 1866 bp, Homo sapiens
ggccattctctcagatataaggcttggaagccagcagctgcgactcccgagacccccccaccagaaggccatggtctccccacggatgtccgggctcctctcccagactgtgatcctagcgctcattttcctcccccagacacggcccgctggcgtcttcgagctgcagatccactctttcgggccgggtccaggccctggggccccgcggtccccctgcagcgcccggctcccctgccgcctcttcttcagagtctgcctgaagcctgggctctcagaggaggccgccgagtccccgtgcgccctgggcgcggcgctgagtgcgcgcggaccggtctacaccgagcagcccggagcgcccgcgcctgatctcccactgcccgacggcctcttgcaggtgcccttccgggacgcctggcctggcaccttctctttcatcatcgaaacctggagagaggagttaggagaccagattggagggcccgcctggagcctgctggcgcgcgtggctggcaggcggcgcttggcagccggaggcccgtgggcccgggacattcagcgcgcaggcgcctgggagctgcgcttctcgtaccgcgcgcgctgcgagccgcctgccgtcgggaccgcgtgcacgcgcctctgccgtccgcgcagcgccccctcgcggtgcggtccgggactgcgcccctgcgcaccgctcgaggacgaatgtgaggcgccgctggtgtgccgagcaggctgcagccctgagcatggcttctgtgaacagcccggtgaatgccgatgcctagagggctggactggacccctctgcacggtccctgtctccaccagcagctgcctcagccccaggggcccgtcctctgctaccaccggatgccttgtccctgggcctgggccctgtgacgggaacccgtgtgccaatggaggcagctgtagtgagacacccaggtcctttgaatgcacctgcccgcgtgggttctacgggctgcggtgtgaggtgagcggggtgacatgtgcagatggaccctgcttcaacggcggcttgtgtgtcgggggtgcagaccctgactctgcctacatctgccactgcccacccggtttccaaggctccaactgtgagaagagggtggaccggtgcagcctgcagccatgccgcaatggtgaggcctggaggcctgaacggcgagggatggggtgggggtcctggatggctcagacagtccagggttggaatcctggctttgactcttctaaccctagggcctggggacctgaccttccacctgcaagcctgtaaaatgggcaaggagacattccctatctcataactattaatatttactgagaatttactgtgtgccaggccctattctaggcactgaggatacagcagggaatgaaacagacaaagtccctggccctgcctgatagagctgaggtgcctggtgtgctgaggataagcagggaagccagtgtggttatactgagatgaggtcagggaggtgactgaggcacatcttgtgggaccccctgggtcacaagaaggggaactttcacttttcccctgagtgagatggagccacaggaaggttctgaggagagaagagacctgatatcggtgctaaaagattaaatgcggcccggcacggcgactcacgcctgtaattccagcactttgggaggccgaggcgggcagatcacctgagctcaggagttggagaccagcccgggccacatggtgaaaccccgtctctactaaaaatacaaaaaattagccgggcgtgatggcaggtgcttgtaatcccagctactcgggaggctaaggcggaagaatcacttgaacccgggaggcggaggttgcag

SEQ ID NO: 40 - DNA, 139 bp, Homo sapiens
ggccattctctcagatataaggcttggaagccagcagctgcgactcccgagacccccccaccagaaggccatggtctccccacggatgtccgggctcctctcccagactgtgatcctagcgctcattttcctcccccag

SEQ ID NO: 41 - DNA, 195 bp, Homo sapiens
cggctcccctgccgcctcttcttcagagtctgcctgaagcctgggctctcagaggaggccgccgagtccccgtgcgccctgggcgcggcgctgagtgcgcgcggaccggtctacaccgagcagcccggagcgcccgcgcctgatctcccactgcccgacggcctcttgcaggtgcccttccgggacgcctggcct

SEQ ID NO: 42 - DNA, 231 bp, Homo sapiens
ggcccgcctggagcctgctggcgcgcgtggctggcaggcggcgcttggcagccggaggcccgtgggcccgggacattcagcgcgcaggcgcctgggagctgcgcttctcgtaccgcgcgcgctgcgagccgcctgccgtcgggaccgcgtgcacgcgcctctgccgtccgcgcagcgccccctcgcggtgcggtccgggactgcgcccctgcgcaccgctcgaggacgaat

SEQ ID NO: 43 - DNA, 218 bp, Homo sapiens
tggtgtgccgagcaggctgcagccctgagcatggcttctgtgaacagcccggtgaatgccgatgcctagagggctggactggacccctctgcacggtccctgtctccaccagcagctgcctcagccccaggggcccgtcctctgctaccaccggatgccttgtccctgggcctgggccctgtgacgggaacccgtgtgccaatggaggcagctgtagt

SEQ ID NO: 44 - DNA, 223 bp, Homo sapiens
gagacacccaggtcctttgaatgcacctgcccgcgtgggttctacgggctgcggtgtgaggtgagcggggtgacatgtgcagatggaccctgcttcaacggcggcttgtgtgtcgggggtgcagaccctgactctgcctacatctgccactgcccacccggtttccaaggctccaactgtgagaagagggtggaccggtgcagcctgcagccatgccgcaatg

SEQ ID NO: 45 - DNA, 703 bp, Homo sapiens
gtgaggcctggaggcctgaacggcgagggatggggtgggggtcctggatggctcagacagtccagggttggaatcctggctttgactcttctaaccctagggcctggggacctgaccttccacctgcaagcctgtaaaatgggcaaggagacattccctatctcataactattaatatttactgagaatttactgtgtgccaggccctattctaggcactgaggatacagcagggaatgaaacagacaaagtccctggccctgcctgatagagctgaggtgcctggtgtgctgaggataagcagggaagccagtgtggttatactgagatgaggtcagggaggtgactgaggcacatcttgtgggaccccctgggtcacaagaaggggaactttcacttttcccctgagtgagatggagccacaggaaggttctgaggagagaagagacctgatatcggtgctaaaagattaaatgcggcccggcacggcgactcacgcctgtaattccagcactttgggaggccgaggcgggcagatcacctgagctcaggagttggagaccagcccgggccacatggtgaaaccccgtctctactaaaaatacaaaaaattagccgggcgtgatggcaggtgcttgtaatcccagctactcgggaggctaaggcggaagaatcacttgaacccgggaggcggaggttgcag

SEQ ID NO: 46 - DNA, 580 bp, Homo sapiens
gcggactctgcctggacctgggccacgccctgcgctgccgctgccgcgccggcttcgcgggtcctcgctgcgagcacgacctggacgactgcgcgggccgcgcctgcgctaacggcggcacgtgtgtggagggcggcggcgcgcaccgctgctcctgcgcgctgggcttcggcggccgcgactgccgcgagcgcgcggacccgtgcgccgcgcgcccctgtgctcacggcggccgctgctacgcccacttctccggcctcgtctgcgcttgcgctcccggctacatgggagcgcggtgtgagttcccagtgcaccccgacggcgcaagcgccttgcccgcggccccgccgggcctcaggcccggggaccctcagcgctaccttttgcctccggctctgggactgctcgtggccgcgggcgtggccggcgctgcgctcttgctggtccacgtgcgccgccgtggccactcccaggatgctgggtctcgcttgctggctgggaccccggagccgtcagtccacgcactcccggatgcactcaacaacctaaggacgcaggagggttccggggatggtccgag

SEQ ID NO: 47 - DNA, 419 bp, Homo sapiens
gcctgacgcgtctcctccatccgcacctggagtcagagcgtggatttttgtatttgctcggtggtgcccagtctctgccccagaggctttggagttcaatcttgaaggggtgtctgggggaactttactgttgcaagttgtaaataatggttatttatatcctattttttctcaccccatctctctagaaacacctataaaggctattattgtgatcagttttgactaacgaggctgaatgctttgtatttgatgttggggctgaggggaagagattttgtctcctgaggagaatagtagacaggactctttatgttgtaggggtcggaaacgactcaaactgctttaaatataaaagaatttatggctgggtgcagtggctcacggctgtaatcccactgaaccactgagtgagatca

SEQ ID NO: 48 - DNA, 11 bp, Homo sapiens
gtcagagccag

SEQ ID NO: 49 - DNA, 58 bp, Homo sapiens
acacggcccgctggcgtcttcgagctgcagatccactctttcgggccgggtccaggcc

SEQ ID NO: 50 - DNA, 29 bp, Homo sapiens
ctggggccccgcggtccccctgcagcgcc

SEQ ID NO: 51 - DNA, 58 bp, Homo sapiens
ggcaccttctctttcatcatcgaaacctggagagaggagttaggagaccagattggag

SEQ ID NO: 52 - DNA, 12 bp, Homo sapiens
gtgaggcgccgc

SEQ ID NO: 53 - DNA, 85 bp, Homo sapiens
ctcgtccgtagattggaatcgccctgaagatgtagaccctcaagggatttatgtcatatctgctccttccatctacgctcgggag

SEQ ID NO: 54 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 55 - protein, 587 aa, Homo sapiens [sequence omitted]

SEQ ID NO: 56 - protein, 587 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREA

SEQ ID NO: 57 - protein, 89 aa, Homo sapiens
MVSPRMSGLLSQTVILALIFLPQTRPAGVFELQIHSFGPGPGPAPLPPLLQSLPEAWALRGGRRVPVRPGRGAECARTGLHRAARSARA

SEQ ID NO: 58 - protein, 67 aa, Homo sapiens
MVSPRMSGLLSQTVILALIFLPQVRARHGPLASSSCRSTLSGRVQALGPRGPPAAPGSPAASSSESA

SEQ ID NO: 59 - protein, 409 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGEAWRPERRGMGWGSWMAQTVQGWNPGFDSSNPRAWGPDLPPASL

SEQ ID NO: 60 - DNA, 18 bp, Artificial Sequence
  Synthetic oligonucleotide
tgtgaacagcccggtgaa

SEQ ID NO: 61 - DNA, 20 bp, Artificial Sequence
  Synthetic oligonucleotide
gacaaggcatccggtggtag

SEQ ID NO: 62 - DNA, 126 bp, Artificial Sequence
  Synthetic oligonucleotide
tgtgaacagcccggtgaatgccgatgcctagagggctggactggacccctctgcacggtccctgtctccaccagcagctgcctcagccccaggggcccgtcctctgctaccaccggatgccttgtc

SEQ ID NO: 63 - protein, 44 aa, Artificial Sequence
  Synthetic peptide
EAWRPERRGMGWGSWMAQTVQGWNPGFDSSNPRAWGPDLPPASL

SEQ ID NO: 64 - DNA, 14 bp, Homo sapiens
agccagacacggcc

SEQ ID NO: 65 - DNA, 20 bp, Artificial Sequence
  Synthetic oligonucleotide
tcattttcctcccccaggtc

SEQ ID NO: 66 - DNA, 21 bp, Artificial Sequence
  Synthetic oligonucleotide
gtggatctgcagctcgaagac

SEQ ID NO: 67 - DNA, 64 bp, Artificial Sequence
  Synthetic oligonucleotide
tcattttcctcccccaggtcagagccagacacggcccgctggcgtcttcgagctgcagatccac

SEQ ID NO: 68 - DNA, 16 bp, Homo sapiens
tcagagccagacacgg

SEQ ID NO: 69 - DNA, 22 bp, Artificial Sequence
  Synthetic oligonucleotide
tcctctcccagactgtgatcct

SEQ ID NO: 70 - DNA, 91 bp, Artificial Sequence
  Synthetic oligonucleotide
tcctctcccagactgtgatcctagcgctcattttcctcccccaggtcagagccagacacggcccgctggcgtcttcgagctgcagatccac

SEQ ID NO: 71 - DNA, 15 bp, Homo sapiens
cctcccccagacacg

SEQ ID NO: 72 - DNA, 17 bp, Artificial Sequence
  Synthetic oligonucleotide
acccggcccgaaagagt

SEQ ID NO: 73 - DNA, 95 bp, Artificial Sequence
  Synthetic oligonucleotide
tcctctcccagactgtgatcctagcgctcattttcctcccccagacacggcccgctggcgtcttcgagctgcagatccactctttcgggccgggt

SEQ ID NO: 74 - DNA, 2359 bp, Homo sapiens
actcccgagacccccccaccagaaggccatggtctccccacggatgtccgggctcctctcccagactgtgatcctagcgctcattttcctcccccagacacggcccgctggcgtcttcgagctgcagatccactctttcgggccgggtccaggccctggggccccgcggtccccctgcagcgcccggctcccctgccgcctcttcttcagagtctgcctgaagcctgggctctcagaggaggccgccgagtccccgtgcgccctgggcgcggcgctgagtgcgcgcggaccggtctacaccgagcagcccggagcgcccgcgcctgatctcccactgcccgacggcctcttgcaggtgcccttccgggacgcctggcctggcaccttctctttcatcatcgaaacctggagagaggagttaggagaccagattggagggcccgcctggagcctgctggcgcgcgtggctggcaggcggcgcttggcagccggaggcccgtgggcccgggacattcagcgcgcaggcgcctgggagctgcgcttctcgtaccgcgcgcgctgcgagccgcctgccgtcgggaccgcgtgcacgcgcctctgccgtccgcgcagcgccccctcgcggtgcggtccgggactgcgcccctgcgcaccgctcgaggacgaatgtgaggcgccgctggtgtgccgagcaggctgcagccctgagcatggcttctgtgaacagcccggtgaatgccgatgcctagagggctggactggacccctctgcacggtccctgtctccaccagcagctgcctcagccccaggggcccgtcctctgctaccaccggatgccttgtccctgggcctgggccctgtgacgggaacccgtgtgccaatggaggcagctgtagtgagacacccaggtcctttgaatgcacctgcccgcgtgggttctacgggctgcggtgtgaggtgagcggggtgacatgtgcagatggaccctgcttcaacggcggcttgtgtgtcgggggtgcagaccctgactctgcctacatctgccactgcccacccggtttccaaggctccaactgtgagaagagggtggaccggtgcagcctgcagccatgccgcaatggcggactctgcctggacctgggccacgccctgcgctgccgctgccgcgccggcttcgcgggtcctcgctgcgagcacgacctggacgactgcgcgggccgcgcctgcgctaacggcggcacgtgtgtggagggcggcggcgcgcaccgctgctcctgcgcgctgggcttcggcggccgcgactgccgcgagcgcgcggacccgtgcgccgcgcgcccctgtgctcacggcggccgctgctacgcccacttctccggcctcgtctgcgcttgcgctcccggctacatgggagcgcggtgtgagttcccagtgcaccccgacggcgcaagcgccttgcccgcggccccgccgggcctcaggcccggggaccctcagcgctaccttttgcctccggctctgggactgctcgtggccgcgggcgtggccggcgctgcgctcttgctggtccacgtgcgccgccgtggccactcccaggatgctgggtctcgcttgctggctgggaccccggagccgtcagtccacgcactcccggatgcactcaacaacctaaggacgcaggagggttccggggatggtccgagctcgtccgtagattggaatcgccctgaagatgtagaccctcaagggatttatgtcatatctgctccttccatctacgctcgggaggtagcgacgccccttttccccccgctacacactgggcgcgctgggcagaggcagcacctgctttttccctacccttcctcgattctgtccgtgaaatgaattgggtagagtctctggaaggttttaagcccattttcagttctaacttactttcatcctattttgcatccctcttatcgttttgagctacctgccatcttctctttgaaaaacctatgggcttgaggaggtcacgatgccgactccgccagagcttttccactgattgtactcagcggggaggcaggggaggcagaggggcagcctctctaatgcttcctactcattttgtttctaggcctgacgcgtctcctccatccgcacctggagtcagagcgtggatttttgtatttgctcggtggtgcccagtctctgccccagaggctttggagttcaatcttgaaggggtgtctgggggaactttactgttgcaagttgtaaataatggttatttatatcctattttttctcaccccatctctctagaaacacctataaaggctattattgtgatcaaaaaaaaaaaaaaaaaaa

SEQ ID NO: 75 - DNA, 2022 bp, Homo sapiens
actcccgagacccccccaccagaaggccatggtctccccacggatgtccgggctcctctcccagactgtgatcctagcgctcattttcctcccccagacacggcccgctggcgtcttcgagctgcagatccactctttcgggccgggtccaggccctggggccccgcggtccccctgcagcgcccggctcccctgccgcctcttcttcagagtctgcctgaagcctgggctctcagaggaggccgccgagtccccgtgcgccctgggcgcggcgctgagtgcgcgcggaccggtctacaccgagcagcccggagcgcccgcgcctgatctcccactgcccgacggcctcttgcaggtgcccttccgggacgcctggcctggcaccttctctttcatcatcgaaacctggagagaggagttaggagaccagattggagggcccgcctggagcctgctggcgcgcgtggctggcaggcggcgcttggcagccggaggcccgtgggcccgggacattcagcgcgcaggcgcctgggagctgcgcttctcgtaccgcgcgcgctgcgagccgcctgccgtcgggaccgcgtgcacgcgcctctgccgtccgcgcagcgccccctcgcggtgcggtccgggactgcgcccctgcgcaccgctcgaggacgaatgtgaggcgccgctggtgtgccgagcaggctgcagccctgagcatggcttctgtgaacagcccggtgaatgccgatgcctagagggctggactggacccctctgcacggtccctgtctccaccagcagctgcctcagccccaggggcccgtcctctgctaccaccggatgccttgtccctgggcctgggccctgtgacgggaacccgtgtgccaatggaggcagctgtagtgagacacccaggtcctttgaatgcacctgcccgcgtgggttctacgggctgcggtgtgaggtgagcggggtgacatgtgcagatggaccctgcttcaacggcggcttgtgtgtcgggggtgcagaccctgactctgcctacatctgccactgcccacccggtttccaaggctccaactgtgagaagagggtggaccggtgcagcctgcagccatgccgcaatggcggactctgcctggacctgggccacgccctgcgctgccgctgccgcgccggcttcgcgggtcctcgctgcgagcacgacctggacgactgcgcgggccgcgcctgcgctaacggcggcacgtgtgtggagggcggcggcgcgcaccgctgctcctgcgcgctgggcttcggcggccgcgactgccgcgagcgcgcggacccgtgcgccgcgcgcccctgtgctcacggcggccgctgctacgcccacttctccggcctcgtctgcgcttgcgctcccggctacatgggagcgcggtgtgagttcccagtgcaccccgacggcgcaagcgccttgcccgcggccccgccgggcctcaggcccggggaccctcagcgctaccttttgcctccggctctgggactgctcgtggccgcgggcgtggccggcgctgcgctcttgctggtccacgtgcgccgccgtggccactcccaggatgctgggtctcgcttgctggctgggaccccggagccgtcagtccacgcactcccggatgcactcaacaacctaaggacgcaggagggttccggggatggtccgagctcgtccgtagattggaatcgccctgaagatgtagaccctcaagggatttatgtcatatctgctccttccatctacgctcgggaggcctgacgcgtctcctccatccgcacctggagtcagagcgtggatttttgtatttgctcggtggtgcccagtctctgccccagaggctttggagttcaatcttgaaggggtgtctgggggaactttactgttgcaagttgtaaataatggttatttatatcctattttttctcaccccatctctctagaaacacctataaaggctattattgtgatcaaaaaaaaaaaaaaaaaaa

SEQ ID NO: 76 - protein, 44 aa, Artificial Sequence
  Synthetic peptide
VRARHGPLASSSCRSTLSGRVQALGPRGPPAAPGSPAASSSESA

SEQ ID NO: 77 - protein, 46 aa, Artificial Sequence
  Synthetic peptide
APLPPLLQSLPEAWALRGGRRVPVRPGRGAECARTGLHRAARSARA

SEQ ID NO: 78 - DNA, 2809 bp, Homo sapiens
ggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcgtgctgccctggctgaaggagaaactccaagatgaggatttgggttttctataaggggtttcctgctggacaggggcgtgggattgaattaaaacagctgcgacaaca

SEQ ID NO: 79 - DNA, 3143 bp, Homo sapiens
ggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggtcagaagggacctctctcctgtcccagcctccccaccttctcagaccagcatgtggcccttaagtccacttgtaacactatacccatggttggggccctgaatgtgactcatagctggctgttcatctctcctgtgacccttcataaggaattcttcctaagccctgtgatcaactatctctaacccttcctcaacttgctcaccctgccatgtgtatccctgcctttagccagtttatcttccttatctcctaccctcatggtcctgtctcttctgcaggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaaca

SEQ ID NO: 80 - DNA, 3262 bp, Homo sapiens
ggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagaggtttgagggcaatgagtgtgggcagtggcctaaggcagaaacagggcaggcggcagcaaggtcaggactaggatgagactaggcagggtgacaaggtgggctgaccgggagtaggagcagttttagggtggcaggcggaaagggggcaagaaaaagcggagttaacccttactaagcatttaccctgggcttccaggcagccctggaagtcaagagaacactcagaaatggggagggagaagcagtggaaatccatatgggttgaggagtaggtaagatgctgcttctgcgggactgggaatgcgctgtttctcagtgacatggtctccgagaccaggagggatacacctaaggcagcctttccctcttgatgacttctacttgtccccccttctcaaagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaaca

SEQ ID NO: 81 - DNA, 3132 bp, Homo sapiens
ggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagtgagtcctccctttcctatctggggagatgccaagtggtcagcatgggccccaaagcaggaaagctcaatgcatgtggctagtaattcgaggtaggcagagcctgcctcaccttaggaccgcatgtcttgcctgcgtgtgtcaagaacgaggctgagctgggtccctagtctgattcctttaggtcagctaagacgcaagcaggaacagccatgcttccaggattaggaattctactgaatgatccatggcaccccactgcctctgcaggttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaaca

SEQ ID NO: 82 - DNA, 2860 bp, Homo sapiens
ggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaagaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaaca

SEQ ID NO: 83 - DNA, 3130 bp, Homo sapiens
ggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggaggtgagaagcatcccctccccctacattgctgtctccctgacggcgcccagcccgaggagtgggcactcggctccggacactgtaactcttgctctctaccttgctcacggggcctcaggcttcagtgcttacctcgatgtctcatacctctgcagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggaatcctcctatccctgaactcgggggaatggaatctcgctgatcttccaggactagctccctgatcattccagcccctctgaacaacagggccccaggaaaatctccaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaaca

SEQ ID NO: 84 - DNA, 2785 bp, Homo sapiens
ggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcagaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaaca

SEQ ID NO: 85 - DNA, 3228 bp, Homo sapiens
ggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataaggtgagaaacgggcatcctaaggaggcactctaggccccaatccttcctaagccacttctgttcattacttctccatgcttcccacctcccctacagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagtgagtcctccctttcctatctggggagatgccaagtggtcagcatgggccccaaagcaggaaagctcaatgcatgtggctagtaattcgaggtaggcagagcctgcctcaccttaggaccgcatgtcttgcctgcgtgtgtcaagaacgaggctgagctgggtccctagtctgattcctttaggtcagctaagacgcaagcaggaacagccatgcttccaggattaggaattctactgaatgatccatggcaccccactgcctctgcaggttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaaca

SEQ ID NO: 86 - DNA, 2631 bp, Homo sapiens
ggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtagaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaaca

SEQ ID NO: 87 - DNA, 1957 bp, Homo sapiens
ggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcggtagggagatacaagggaataaagaacacaactctc

SEQ ID NO: 88 - DNA, 2238 bp, Homo sapiens
ggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggtcagaagggacctctctcctgtcccagcctccccaccttctcagaccagcatgtggcccttaagtccacttgtaacactatacccatggttggggccctgaatgtgactcatagctggctgttcatctctcctgtgacccttcataaggaattcttcctaagccctgtgatcaactatctctaacccttcctcaacttgctcaccctgccatgtgtatccctgcctttagccagtttatcttccttatctcctaccctcatggtcctgtctcttctgcaggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcggtagggagatacaagggaataaagaacacaactctc

SEQ ID NO: 89 - DNA, 577 bp, Homo sapiens
ggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggag

SEQ ID NO: 90 - DNA, 156 bp, Homo sapiens
gtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacac

SEQ ID NO: 91 - DNA, 400 bp, Homo sapiens
gtttgagggcaatgagtgtgggcagtggcctaaggcagaaacagggcaggcggcagcaaggtcaggactaggatgagactaggcagggtgacaaggtgggctgaccgggagtaggagcagttttagggtggcaggcggaaagggggcaagaaaaagcggagttaacccttactaagcatttaccctgggcttccaggcagccctggaagtcaagagaacactcagaaatggggagggagaagcagtggaaatccatatgggttgaggagtaggtaagatgctgcttctgcgggactgggaatgcgctgtttctcagtgacatggtctccgagaccaggagggatacacctaaggcagcctttccctcttgatgacttctacttgtccccccttctcaaag

SEQ ID NO: 92 - DNA, 186 bp, Homo sapiens
caatccactgtccaagaccacacgacttcgagaacggggaatactggccccggtctccctactacaatgtgagtgatgagatctctttccactgctatgacggttacactctccggggctctgccaatcgcacctgccaagtgaatggccggtggagtgggcagacagcgatctgtgacaacggag

SEQ ID NO: 93 - DNA, 155 bp, Homo sapiens
gtgagaagcatcccctccccctacattgctgtctccctgacggcgcccagcccgaggagtgggcactcggctccggacactgtaactcttgctctctaccttgctcacggggcctcaggcttcagtgcttacctcgatgtctcatacctctgcag

SEQ ID NO: 94 - DNA, 174 bp, Homo sapiens
cggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaag

SEQ ID NO: 95 - DNA, 132 bp, Homo sapiens
accacaagttgaagtcagggactaacaccaagaaggccctccaggcagtgtacagcatgatgagctggccagatgacgtccctcctgaaggctggaaccgcacccgccatgtcatcatcctcatgactgatg

SEQ ID NO: 96 - DNA, 281 bp, Homo sapiens
gtcagaagggacctctctcctgtcccagcctccccaccttctcagaccagcatgtggcccttaagtccacttgtaacactatacccatggttggggccctgaatgtgactcatagctggctgttcatctctcctgtgacccttcataaggaattcttcctaagccctgtgatcaactatctctaacccttcctcaacttgctcaccctgccatgtgtatccctgcctttagccagtttatcttccttatctcctaccctcatggtcctgtctcttctgcag

SEQ ID NO: 97 - DNA, 145 bp, Homo sapiens
gtagggagatacaagggaataaagaacacaactctcctcaggttcccctgaagtaattcattcttcctctacacctgaagctctagttgcctggaaagccttcttcattcctccttctctacctcagtgtcactattcttgtttc

SEQ ID NO: 98 - DNA, 154 bp, Homo sapiens
gaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcag

SEQ ID NO: 99 - DNA, 270 bp, Homo sapiens
gtgagtcctccctttcctatctggggagatgccaagtggtcagcatgggccccaaagcaggaaagctcaatgcatgtggctagtaattcgaggtaggcagagcctgcctcaccttaggaccgcatgtcttgcctgcgtgtgtcaagaacgaggctgagctgggtccctagtctgattcctttaggtcagctaagacgcaagcaggaacagccatgcttccaggattaggaattctactgaatgatccatggcaccccactgcctctgcag

SEQ ID NO: 100 - DNA, 78 bp, Homo sapiens
gtacctgcagatctacggggtcctggagcaccctgaagactcaagaccaaaagactgtcaggaaggcagagtgcagag

SEQ ID NO: 101 - DNA, 68 bp, Homo sapiens
actccttcatgtacgacacccctcaagaggtggccgaagctttcctgtcttccctgacagagaccata

SEQ ID NO: 102 - DNA, 34 bp, Homo sapiens
gaaggagtcgatgctgaggatgggcacggcccag

SEQ ID NO: 103 - DNA, 84 bp, Homo sapiens
gggaacaacagaagcggaagatcgtcctggacccttcaggctccatgaacatctacctggtgctagatggatcagacagcattg

SEQ ID NO: 104 - DNA, 18 bp, Homo sapiens
gggccagcaacttcacag

SEQ ID NO: 105 - DNA, 35 bp, Homo sapiens
gagccaaaaagtgtctagtcaacttaattgagaag

SEQ ID NO: 106 - DNA, 113 bp, Homo sapiens
gtggaatcctcctatccctgaactcgggggaatggaatctcgctgatcttccaggactagctccctgatcattccagcccctctgaacaacagggccccaggaaaatctccag

SEQ ID NO: 107 - DNA, 56 bp, Homo sapiens
gtggcaagttatggtgtgaagccaagatatggtctagtgacatatgccacataccc

SEQ ID NO: 108 - DNA, 30 bp, Homo sapiens
caaaatttgggtcaaagtgtctgaagcaga

SEQ ID NO: 109 - DNA, 24 bp, Homo sapiens
cagcagtaatgcagactgggtcac

SEQ ID NO: 110 - DNA, 7 bp, Homo sapiens
gaagcag

SEQ ID NO: 111 - DNA, 22 bp, Homo sapiens
ctcaatgaaatcaattatgaag

SEQ ID NO: 112 - DNA, 83 bp, Homo sapiens
gattgcacaacatgggcggggacccaattactgtcattgatgagatccgggacttgctatacattggcaaggatcgcaaaaac

SEQ ID NO: 113 - DNA, 19 bp, Homo sapiens
ccaagggaggattatctgg

SEQ ID NO: 114 - DNA, 35 bp, Homo sapiens
atgtctatgtgtttggggtcgggcctttggtgaac

SEQ ID NO: 115 - DNA, 9 bp, Homo sapiens
caagtgaac

SEQ ID NO: 116 - DNA, 94 bp, Homo sapiens
atcaatgctttggcttccaagaaagacaatgagcaacatgtgttcaaagtcaaggatatggaaaacctggaagatgttttctaccaaatgatcg

SEQ ID NO: 117 - DNA, 98 bp, Homo sapiens
atgaaagccagtctctgagtctctgtggcatggtttgggaacacaggaagggtaccgattaccacaagcaaccatggcaggccaagatctcagtcatt

SEQ ID NO: 118 - DNA, 64 bp, Homo sapiens
cgcccttcaaagggacacgagagctgtatgggggctgtggtgtctgagtactttgtgctgacag

SEQ ID NO: 119 - DNA, 54 bp, Homo sapiens
cagcacattgtttcactgtggatgacaaggaacactcaatcaaggtcagcgtag

SEQ ID NO: 120 - DNA, 77 bp, Homo sapiens
gcccatttgtctcccctgcaccgagggaacaactcgagctttgaggcttcctccaactaccacttgccagcaacaaa

SEQ ID NO: 121 - DNA, 2 bp, Homo sapiens
ag

SEQ ID NO: 122 - DNA, 31 bp, Homo sapiens
gaagagctgctccctgcacaggatatcaaag

SEQ ID NO: 123 - DNA, 68 bp, Homo sapiens
ctctgtttgtgtctgaggaggagaaaaagctgactcggaaggaggtctacatcaagaatggggataag

SEQ ID NO: 124 - DNA, 96 bp, Homo sapiens
gtgagaaacgggcatcctaaggaggcactctaggccccaatccttcctaagccacttctgttcattacttctccatgcttcccacctcccctacag

SEQ ID NO: 125 - DNA, 97 bp, Homo sapiens
aaaggcagctgtgagagagatgctcaatatgccccaggctatgacaaagtcaaggacatctcagaggtggtcacccctcggttcctttgtactggag

SEQ ID NO: 126 - DNA, 36 bp, Homo sapiens
gagtgagtccctatgctgaccccaatacttgcagag

SEQ ID NO: 127 - DNA, 50 bp, Homo sapiens
gtgattctggcggccccttgatagttcacaagagaagtcgtttcattcaa

SEQ ID NO: 128 - DNA, 50 bp, Homo sapiens
gttggtgtaatcagctggggagtagtggatgtctgcaaaaaccagaagcg

SEQ ID NO: 129 - DNA, 53 bp, Homo sapiens
gcaaaagcaggtacctgctcacgcccgagactttcacatcaacctctttcaag

SEQ ID NO: 130 - DNA, 107 bp, Homo sapiens
tgctgccctggctgaaggagaaactccaagatgaggatttgggttttctataaggggtttcctgctggacaggggcgtgggattgaattaaaacagctgcgacaaca

SEQ ID NO: 131 - protein, 764 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 132 - protein, 621 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGKDATEGPGLHLCSPGNTSHFLQILHSTHPQCSPIPCTPDQSGMGEDVKLGMTRGQRQEAAHKEVVPTLLLQEGRSGTWR

SEQ ID NO: 133 - protein, 764 aa, Homo sapiens
MGSNLSPQLCLMPFILGLLSGGVTTTPWSLAQPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 134 - protein, 740 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRAALAEGETPR

SEQ ID NO: 135 - protein, 450 aa, Homo sapiens [sequence omitted]

SEQ ID NO: 136 - protein, 744 aa, Homo sapiens [sequence omitted]

SEQ ID NO: 137 - protein, 633 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQRRAAPCTGYQSSVCV

SEQ ID NO: 138 - protein, 608 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRGRAAPCTGYQSSVCV

SEQ ID NO: 139 - protein, 662 aa, Homo sapiens [sequence omitted]

SEQ ID NO: 140 - protein, 687 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVEEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 141 - protein, 481 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIGREIQGNKEHNS

SEQ ID NO: 142 - protein, 289 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGGEKHPLPLHCCLPDGAQPEEWALGSGHCNSCSLPCSRGLRLQCLPRCLIPLQRGTAPTRASPLAQGRWAASTALKTASPTTAAGGLPCVAPSGERVRKVALGAGRSLPAKTPSCTTPLKRWPKLSCLP

SEQ ID NO: 143 - protein, 122 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRGLRAMSVGSGLRQKQGRRQQGQD

SEQ ID NO: 144 - DNA, 21 bp, Artificial Sequence
  Synthetic oligonucleotide
gtttgagggcaatgagtgtgg

SEQ ID NO: 145 - DNA, 21 bp, Artificial Sequence
  Synthetic oligonucleotide
aaactgctcctactcccggtc

SEQ ID NO: 146 - DNA, 123 bp, Artificial Sequence
  Synthetic oligonucleotide
gtttgagggcaatgagtgtgggcagtggcctaaggcagaaacagggcaggcggcagcaaggtcaggactaggatgagactaggcagggtgacaaggtgggctgaccgggagtaggagcagttt

SEQ ID NO: 147 - DNA, 22 bp, Artificial Sequence
  Synthetic oligonucleotide
ctacattgctgtctccctgacg

SEQ ID NO: 148 - DNA, 22 bp, Artificial Sequence
  Synthetic oligonucleotide
aggtaagcactgaagcctgagg

SEQ ID NO: 149 - DNA, 114 bp, Artificial Sequence
  Synthetic oligonucleotide
ctacattgctgtctccctgacggcgcccagcccgaggagtgggcactcggctccggacactgtaactcttgctctctaccttgctcacggggcctcaggcttcagtgcttacct

SEQ ID NO: 150 - DNA, 21 bp, Artificial Sequence
  Synthetic oligonucleotide
aggcaacacctcccactttct

SEQ ID NO: 151 - DNA, 19 bp, Artificial Sequence
  Synthetic oligonucleotide
ttcacgtcttcccccatcc

SEQ ID NO: 152 - DNA, 101 bp, Artificial Sequence
  Synthetic oligonucleotide
aggcaacacctcccactttctacagatcctacactccacccatcctcaatgcagccccattccttgcaccccagaccagtcagggatgggggaagacgtga

SEQ ID NO: 153 - DNA, 18 bp, Artificial Sequence
  Synthetic oligonucleotide
gaagagctgctccctgca

SEQ ID NO: 154 - DNA, 22 bp, Artificial Sequence
  Synthetic oligonucleotide
ccccattcttgatgtagacctc

SEQ ID NO: 155 - DNA, 94 bp, Artificial Sequence
  Synthetic oligonucleotide
gaagagctgctccctgcacaggatatcaaagctctgtttgtgtctgaggaggagaaaaagctgactcggaaggaggtctacatcaagaatgggg

SEQ ID NO: 156 - DNA, 3362 bp, Homo sapiens
gtgctgtgcggcgcggtctcagggaaggtggggctatggcagctgctagggaccctccggaagtatcgctgcgagaagccacccagcgaaaattgcggaggttttccgagctaagaggcaaacttgtagcacgtggagaattctgggacatagttgcaataacagcggctgatgaaaaacaggaacttgcttacaaccaacagctgtcagaaaagctgaaaagaaaggagttaccccttggagttcaatatcacgtttttgtagatcctgctggagccaaaattggaaatggaggatcaacactttgtgcccttcaatgtttggaaaagctatatggagataaatggaattcttttaccatcttattaattcactctgatgaatggaagaaaaaagtcagtgaatcatatgttatcacaatagaaagattagaagatgacctgcagatcaaggaaaaagaactgacagaactaaggaatatatttggctctgatgaagccttcagtaaagtcaatttaaattaccgcactgaaaatgggctgtctctacttcatttatgttgcatttgtggaggcaagaaatcacatattcgaactcttatgttgaaagggctccgcccatctcgactgacaagaaatggatttacagccttgcatttagcagtttacaaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggttcttttgggaaagtatataaaggacgatgcagaaataaaatagtggctataaaacgttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagaggattcttgatttgcagtctaaattaattattgcagtagatgttgccaaaggcatggagtaccttcacaacctgacacagccaattatacatcgtgacttgaacaggtctgccattacctctaggatctggatcacccatagtatttgcatctggaggggagctcattactttaacagggaagaatgcaatttcaggtgtatgcttacttctgccatcctaaaagaatcaagatttctacagtctctggatgaagacaacatgacaaaacaacctgggaacctccgttggatggctcctgaggtgttcacgcagtgcactcggtacaccatcaaagcagatgtcttcagctatgctctgtgtctgtgggaaattctcactggcgaaattccattcgctcatctcaagccagcggctgcggcagcagacatggcttaccaccacatcagacctcccattggctattccattcccaagcccatatcatctctgctgatacgagggtggaacgcatgtcctgaaggaagacccgaattttctgaagttgtcatgaagttagaagagtgtctctgcaacattgagctgatgtctcctgcatcaagtaacagcagtgggtctctctcaccttcttcttcttctgattgcctggtgaaccggggaggacctggccggagtcatgtggcagcattaagaagtcgtttcgaattggaatatgctctaaatgcaaggtcctatgctgctttgtcccaaagtgctggacaatattcctctcaaggtctgtctttggaggagatgaaaagaagtcttcaatacacacccattgacaaatatggctatgtatccgatcccatgagctcaatgcattttcattcttgccgaaatagtagcagctttgaggacagcagctgacagcattcggcgtatacctaaggagagttttttccccgaactgacagcaacgattccaaccacggcaagctggcttccaactataacattttactctcaaaggtctccttaaattgggcttgtttttacttgtcctatttaattccccactattagcaggctttggatttgtgcctaaggaataatatgcaaaagaaccaagacagaatgtatatgaagaattgtttttaattttgtaaattaaaaaaaaatttagatcgttacttggaaatggagcctaagtctgtggtggacagataataattatgttttcctgggctgaattatgtagacttgtgtttgacagctatgggtttatttcttagaacattgttcattttcttttctcattatgttacttctagtgttcacctctgtgattaaagattctttggtgaaatagaaaattga

SEQ ID NO: 157 - DNA, 3412 bp, Homo sapiens
cttgaaaacaggtctatgtagttttaacagctcttttgtgaacttcggatgtaactgaatcattttctagatttatctgttaaataaatggtccatgaaagtatgtatgggtagataattgatggaagcaaataatcctattgaaacatttgctatttatttttgatacagttcttttaaacagactgattagttccctttatgatttcaccctcgaaagcacacacgattttgttctttatcattgttgcatgacattccatttggcagttgggcttatttttagccagactatcaaagtatttttcaactggactgtcactgcacttgaacttggaatcttataacttgaagaactgccctggagaaaggaagaaacttataataaatgggaaattataaatctagaccaacccaaacttgtactgatgaatggaagaaaaaagtcagtgaatcatatgttatcacaatagaaagattagaagatgacctgcagatcaaggaaaaagaactgacagaactaaggaatatatttggctctgatgaagccttcagtaaagtcaatttaaattaccgcactgaaaatgggctgtctctacttcatttatgttgcatttgtggaggcaagaaatcacatattcgaactcttatgttgaaagggctccgcccatctcgactgacaagaaatggatttacagccttgcatttagcagtttacaaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggttcttttgggaaagtatataaaggacgatgcagaaataaaatagtggctataaaacgttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagaggattcttgatttgcagtctaaattaattattgcagtagatgttgccaaaggcatggagtaccttcacaacctgacacagccaattatacatcgtgacttgaacaggtctgccattacctctaggatctggatcacccatagtatttgcatctggaggggagctcattactttaacagggaagaatgcaatttcaggtgtatgcttacttctgccatcctaaaagaatcaagatttctacagtctctggatgaagacaacatgacaaaacaacctgggaacctccgttggatggctcctgaggtgttcacgcagtgcactcggtacaccatcaaagcagatgtcttcagctatgctctgtgtctgtgggaaattctcactggcgaaattccattcgctcatctcaagccagcggctgcggcagcagacatggcttaccaccacatcagacctcccattggctattccattcccaagcccatatcatctctgctgatacgagggtggaacgcatgtcctgaaggaagacccgaattttctgaagttgtcatgaagttagaagagtgtctctgcaacattgagctgatgtctcctgcatcaagtaacagcagtgggtctctctcaccttcttcttcttctgattgcctggtgaaccggggaggacctggccggagtcatgtggcagcattaagaagtcgtttcgaattggaatatgctctaaatgcaaggtcctatgctgctttgtcccaaagtgctggacaatattcctctcaaggtctgtctttggaggagatgaaaagaagtcttcaatacacacccattgacaaatatggctatgtatccgatcccatgagctcaatgcattttcattcttgccgaaatagtagcagctttgaggacagcagctgacagcattcggcgtatacctaaggagagttttttccccgaactgacagcaacgattccaaccacggcaagctggcttccaactataacattttactctcaaaggtctccttaaattgggcttgtttttacttgtcctatttaattccccactattagcaggctttggatttgtgcctaaggaataatatgcaaaagaaccaagacagaatgtatatgaagaattgtttttaattttgtaaattaaaaaaaaatttagatcgttacttggaaatggagcctaagtctgtggtggacagataataattatgttttcctgggctgaattatgtagacttgtgtttgacagctatgggtttatttcttagaacattgttcattttcttttctcattatgttacttctagtgttcacctctgtgattaaagattctttggtgaaatagaaaattga

SEQ ID NO: 158 - DNA, 3420 bp, Homo sapiens
cttgaaaacaggtctatgtagttttaacagctcttttgtgaacttcggatgtaactgaatcattttctagatttatctgttaaataaatggtccatgaaagtatgtatgggtagataattgatggaagcaaataatcctattgaaacatttgctatttatttttgatacagttcttttaaacagactgattagttccctttatgatttcaccctcgaaagcacacacgattttgttctttatcattgttgcatgacattccatttggcagttgggcttatttttagccagactatcaaagtatttttcaactggactgtcactgcacttgaacttggaatcttataacttgaagaactgccctggagaaaggaagaaacttataataaatgggaaattataaatctagaccaacccaaacttgtactgatgaatggaagaaaaaagtcagtgaatcatatgttatcacaatagaaagattagaagatgacctgcagatcaaggaaaaagaactgacagaactaaggaatatatttggctctgatgaagccttcagtaaagtcaatttaaattaccgcactgaaaatgggctgtctctacttcatttatgttgcatttgtggaggcaagaaatcacatattcgaactcttatgttgaaagggctccgcccatctcgactgacaagaaatggatttacagccttgcatttagcagtttacaaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggttcttttgggaaagtatataaaggacgatgcagaaataaaatagtggctataaaacgttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagaggattcttgatttgcagtctaaattaattattgcagtagatgttgccaaaggcatggagtaccttcacaacctgacacagccaattatacatcgtgacttgaacagtcacaatattcttctctatgaggatgggcatgctgtggtggcagattttggagaatcaagatttctacagtctctggatgaagacaacatgacaaaacaacctgggaacctccgttggatggctcctgaggtgttcacgcagtgcactcggtacaccatcaaagcagatgtcttcagctatgctctgtgtctgtgggaaattctcactggcgaaattccattcgctcatctcaagccagcggctgcggcagcagacatggcttaccaccacatcagacctcccattggctattccattcccaagcccatatcatctctgctgatacgagggtggaacgcatgtcctgaaggaagacccgaattttctgaagttgtcatgaagttagaagagtgtctctgcaacattgagctgatgtctcctgcatcaagtaacagcagtgggtctctctcaccttcttcttcttctgattgcctggtgaaccggggaggacctggccggagtcatgtggcagcattaagaagtcgtttcgaattggaatatgctctaaatgcaaggtcctatgctgctttgtcccaaagtgctggacaatattcctctcaaggtctgtctttggaggagatgaaaagaagtcttcaatacacacccattgacaaatatgatgtaacttcataaacacaaaacatagggcatcctttgaaacatttgctttgaccagaagtcttcccttttgtggctatgtatccgatcccatgagctcaatgcattttcattcttgccgaaatagtagcagctttgaggacagcagctgacagcattcggcgtatacctaaggagagttttttccccgaactgacagcaacgattccaaccacggcaagctggcttccaactataacattttactctcaaaggtctccttaaattgggcttgtttttacttgtcctatttaattccccactattagcaggctttggatttgtgcctaaggaataatatgcaaaagaaccaagacagaatgtatatgaagaattgtttttaattttgtaaattaaaaaaaaatttagatcgttacttggaaatggagcctaagtctgtggtggacagataataattatgttttcctgggctgaattatgtagacttgtgtttgacagctatgggtttatttcttagaacattgttcattttcttttctcattatgttacttctagtgttcacctctgtgattaaagattctttggtgaaatagaaaattga

SEQ ID NO: 159 - DNA, 2670 bp, Homo sapiens
ccttctccatgtggcctgcattttcttataccatggctgacacaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggttcttttgggaaagtatataaaggacgatgcagaaataaaatagtggctataaaacgttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagaggattcttgatttgcagtctaaattaattattgcagtagatgttgccaaaggcatggagtaccttcacaacctgacacagccaattatacatcgtgacttgaacagtcacaatattcttctctatgaggatgggcatgctgtggtggcagattttggagaatcaagatttctacagtctctggatgaagacaacatgacaaaacaacctgggaacctccgttggatggctcctgaggtgttcacgcagtgcactcggtacaccatcaaagcagatgtcttcagctatgctctgtgtctgtgggaaattctcactggcgaaattccattcgctcatctcaagccagcggctgcggcagcagacatggcttaccaccacatcagacctcccattggctattccattcccaagcccatatcatctctgctgatacgagggtggaacgcatgtcctgaaggaagacccgaattttctgaagttgtcatgaagttagaagagtgtctctgcaacattgagctgatgtctcctgcatcaagtaacagcagtgggtctctctcaccttcttcttcttctgattgcctggtgaaccggggaggacctggccggagtcatgtggcagcattaagaagtcgtttcgaattggaatatgctctaaatgcaaggtcctatgctgctttgtcccaaagtgctggacaatattcctctcaaggtctgtctttggaggagatgaaaagaagtcttcaatacacacccattgacaaatatggctatgtatccgatcccatgagctcaatgcattttcattcttgccgaaatagtagcagctttgaggacagcagctgacagcattcggcgtatacctaaggagagttttttccccgaactgacagcaacgattccaaccacggcaagctggcttccaactataacattttactctcaaaggtctccttaaattgggcttgtttttacttgtcctatttaattccccactattagcaggctttggatttgtgcctaaggaataatatgcaaaagaaccaagacagaatgtatatgaagaattgtttttaattttgtaaattaaaaaaaaatttagatcgttacttggaaatggagcctaagtctgtggtggacagataataattatgttttcctgggctgaattatgtagacttgtgtttgacagctatgggtttatttcttagaacattgttcattttcttttctcattatgttacttctagtgttcacctctgtgattaaagattctttggtgaaatagaaaattga

SEQ ID NO: 160 - DNA, 2736 bp, Homo sapiens
ccttctccatgtggcctgcattttcttataccatggctgacacaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggttcttttgggaaagtatataaaggacgatgcagaaataaaatagtggctataaaacgttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagaggattcttgatttgcagtctaaattaattattgcagtagatgttgccaaaggcatggagtaccttcacaacctgacacagccaattatacatcgtgacttgaacaggtctgccattacctctaggatctggatcacccatagtatttgcatctggaggggagctcattactttaacagggaagaatgcaatttcaggtgtatgcttacttctgccatcctaaaagaatcaagatttctacagtctctggatgaagacaacatgacaaaacaacctgggaacctccgttggatggctcctgaggtgttcacgcagtgcactcggtacaccatcaaagcagatgtcttcagctatgctctgtgtctgtgggaaattctcactggcgaaattccattcgctcatctcaagccagcggctgcggcagcagacatggcttaccaccacatcagacctcccattggctattccattcccaagcccatatcatctctgctgatacgagggtggaacgcatgtcctgaaggaagacccgaattttctgaagttgtcatgaagttagaagagtgtctctgcaacattgagctgatgtctcctgcatcaagtaacagcagtgggtctctctcaccttcttcttcttctgattgcctggtgaaccggggaggacctggccggagtcatgtggcagcattaagaagtcgtttcgaattggaatatgctctaaatgcaaggtcctatgctgctttgtcccaaagtgctggacaatattcctctcaaggtctgtctttggaggagatgaaaagaagtcttcaatacacacccattgacaaatatggctatgtatccgatcccatgagctcaatgcattttcattcttgccgaaatagtagcagctttgaggacagcagctgacagcattcggcgtatacctaaggagagttttttccccgaactgacagcaacgattccaaccacggcaagctggcttccaactataacattttactctcaaaggtctccttaaattgggcttgtttttacttgtcctatttaattccccactattagcaggctttggatttgtgcctaaggaataatatgcaaaagaaccaagacagaatgtatatgaagaattgtttttaattttgtaaattaaaaaaaaatttagatcgttacttggaaatggagcctaagtctgtggtggacagataataattatgttttcctgggctgaattatgtagacttgtgtttgacagctatgggtttatttcttagaacattgttcattttcttttctcattatgttacttctagtgttcacctctgtgattaaagattctttggtgaaatagaaaattga

SEQ ID NO: 161 - DNA, 2750 bp, Homo sapiens
gtgctgtgcggcgcggtctcagggaaggtggggctatggcagctgctagggaccctccggaagtatcgctgcgagaagccacccagcgaaaattgcggaggttttccgagctaagaggcaaacttgtagcacgtggagaattctgggacatagttgcaataacagcggctgatgaaaaacaggaacttgcttacaaccaacagctgtcagaaaagctgaaaagaaaggagttaccccttggagttcaatatcacgtttttgtagatcctgctggagccaaaattggaaatggaggatcaacactttgtgcccttcaatgtttggaaaagctatatggagataaatggaattcttttaccatcttattaattcactctgatgaatggaagaaaaaagtcagtgaatcatatgttatcacaatagaaagattagaagatgacctgcagatcaaggaaaaagaactgacagaactaaggaatatatttggctctgatgaagccttcagtaaagtcaatttaaattaccgcactgaaaatgggctgtctctacttcatttatgttgcatttgtggaggcaagaaatcacatattcgaactcttatgttgaaagggctccgcccatctcgactgacaagaaatggatttacagccttgcatttagcagtttacaaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggttcttttgggaaagtatataaaggacgatgcagaaataaaatagtggctataaaacgttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagaggattcttgatttgcagtctaaattaattattgcagtagatgttgccaaaggcatggagtaccttcacaacctgacacagccaattatacatcgtgacttgaacagtcacaatattcttctctatgaggatgggcatgctgtggtggcagattttggagaatcaagatttctacagtctctggatgaagacaacatgacaaaacaacctgggaacctccgttggatggctcctgaggtgttcacgcagtgcactcggtacaccatcaaagcagatgtcttcagctatgctctgtgtctgtgggaaattctcactggcgaaattccattcgctcatctcaagccagcggctgcggcagcagacatggcttaccaccacatcagacctcccattggctattccattcccaagcccatatcatctctgctgatacgagggtggaacgcatgtcctgaagcaaaatccagaccaagtcattacccagtctcatctgtgtacacagaaactcttaagaagaaaaatgaagatcgttttgggatgtggattgagtatctcagaagataacctcttatcctggccattcaacctgatgtgttacatgtttatttgtttagaatcttccatcactaccaaaatgttagctccatgaaagcggtcctttttgtttattttgttcactgctataacactaacatctaaagctggatgcttaagaatgtttgttgagtaaatgaatgaataccagta

SEQ ID NO: 162 - DNA, 2800 bp, Homo sapiens
cttgaaaacaggtctatgtagttttaacagctcttttgtgaacttcggatgtaactgaatcattttctagatttatctgttaaataaatggtccatgaaagtatgtatgggtagataattgatggaagcaaataatcctattgaaacatttgctatttatttttgatacagttcttttaaacagactgattagttccctttatgatttcaccctcgaaagcacacacgattttgttctttatcattgttgcatgacattccatttggcagttgggcttatttttagccagactatcaaagtatttttcaactggactgtcactgcacttgaacttggaatcttataacttgaagaactgccctggagaaaggaagaaacttataataaatgggaaattataaatctagaccaacccaaacttgtactgatgaatggaagaaaaaagtcagtgaatcatatgttatcacaatagaaagattagaagatgacctgcagatcaaggaaaaagaactgacagaactaaggaatatatttggctctgatgaagccttcagtaaagtcaatttaaattaccgcactgaaaatgggctgtctctacttcatttatgttgcatttgtggaggcaagaaatcacatattcgaactcttatgttgaaagggctccgcccatctcgactgacaagaaatggatttacagccttgcatttagcagtttacaaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggttcttttgggaaagtatataaaggacgatgcagaaataaaatagtggctataaaacgttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagaggattcttgatttgcagtctaaattaattattgcagtagatgttgccaaaggcatggagtaccttcacaacctgacacagccaattatacatcgtgacttgaacagtcacaatattcttctctatgaggatgggcatgctgtggtggcagattttggagaatcaagatttctacagtctctggatgaagacaacatgacaaaacaacctgggaacctccgttggatggctcctgaggtgttcacgcagtgcactcggtacaccatcaaagcagatgtcttcagctatgctctgtgtctgtgggaaattctcactggcgaaattccattcgctcatctcaagccagcggctgcggcagcagacatggcttaccaccacatcagacctcccattggctattccattcccaagcccatatcatctctgctgatacgagggtggaacgcatgtcctgaagcaaaatccagaccaagtcattacccagtctcatctgtgtacacagaaactcttaagaagaaaaatgaagatcgttttgggatgtggattgagtatctcagaagataacctcttatcctggccattcaacctgatgtgttacatgtttatttgtttagaatcttccatcactaccaaaatgttagctccatgaaagcggtcctttttgtttattttgttcactgctataacactaacatctaaagctggatgcttaagaatgtttgttgagtaaatgaatgaataccagta

SEQ ID NO: 163 - DNA, 7566 bp, Homo sapiens
gtgctgtgcggcgcggtctcagggaaggtggggctatggcagctgctagggaccctccggaagtatcgctgcgagaagccacccagcgaaaattgcggaggttttccgagctaagaggcaaacttgtagcacgtggagaattctgggacatagttgcaataacagcggctgatgaaaaacaggaacttgcttacaaccaacagctgtcagaaaagctgaaaagaaaggagttaccccttggagttcaatatcacgtttttgtagatcctgctggagccaaaattggaaatggaggatcaacactttgtgcccttcaatgtttggaaaagctatatggagataaatggaattcttttaccatcttattaattcactctgatgaatggaagaaaaaagtcagtgaatcatatgttatcacaatagaaagattagaagatgacctgcagatcaaggaaaaagaactgacagaactaaggaatatatttggctctgatgaagccttcagtaaagtcaatttaaattaccgcactgaaaatgggctgtctctacttcatttatgttgcatttgtggaggcaagaaatcacatattcgaactcttatgttgaaagggctccgcccatctcgactgacaagaaatggatttacagccttgcatttagcagtttacaaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggttcttttgggaaagtatataaaggacgatgcagaaataaaatagtggctataaaacgttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagaggattcttgatttgcagtctaaattaattattgcagtagatgttgccaaaggcatggagtaccttcacaacctgacacagccaattatacatcgtgacttgaacaggtatttttttcctaaataatgaactcagaagggtatgactaactgggagtttaagacagatttcagtgaagatacattttagacttattgcagatcagggtactctgtggttaagaggacaggctaccataagcttaggcgatagaaagttttgtcattgtcttttaaggagtagaaagctgtataactgcttctcagggatcttttaaatctatgaagaatattcacaaatatagtaatatctgtaagatgcaccagctaactagttcccaggtcagtaaggtcacatcaagcagtgagctaaatggctattaaaaaaatgatatgtctcctataaagtgaagtgttggagtagctcctgatataaaattatctgaaacgtactttgtgctaccgtaaattcagcttaaccacaacagcttatttacctagttatcaggtaaagaggatttcatgtgaaacaaatataaaatcagagtgttccagtggtggaaccccaagtgacctcaaaacttttcctaaatagtgaaaactttctctgagtaaagtcttataggaaaggccaatatacaaaatggagcagtgttgggtgaaggtaaagtggggaggagaaaactctatttacttcccatttcccactgctaaggagagagattcctgcaccacctctgtggaagcccaaaggtgctgtagagaatagttggacaaacactgatttagtttatgaagcaagtcagattattccacatcaatcactttcattttatgatagtcaataagattcaaatagattgtgtatatagtcctttatagaagctttacagttctgaagctagtaaatattgattactctagtcagtcatttggtcaacaactatgtttgaacatatatgtttaaagactattttcagcacagtagagcttacaaagtgggacgttcatagactacattcagggagttttcacactctatgtagaggagaaaaatatttacctaaatgtatttgaggtataaaataataagttatatacaagatttagtacaaaaatttcaggaacaagaatattaattgcttctgaggattctaacaacaactctatggaagggatgacatatgagcttggtcctgaaagatgggtagtttggaagagcatcctataggcttagggaagacataataaaatgcatagagattagaaagttaaaggtatatataggaactatagtttcattttagtatacaaagtgtataaatgtatgtaatgtgtataatgtaagaagtggacaactggaaaaaggtatagaaaaatagaaaaagagcacagacaccagtgaatgctattactaaggtatttcagattttttttcctgtaaggagaccataaagaggtttgagctgaaaatcacaaatttggagctatattattgaaagatgaatccagcatctggtaaaatatcaacttgaagggaaagtcaagaaactagtgagggagttgttgcagtaatctagataagaagtaacgaaaagcagaatttcaccggtggcagtggatatgaaaggaagagacatgtcaactttaaataacaaaatacagagtttattcaagctcaaagcttgaggatcattacccatgagcatggactcaagttgtcctgaatacatactccaattagcagtggttacaagatagtttttaagaaaaaaaaaaaaaaaaaaaaaagcagtttttaagttgtttacaaagaatttacataaataatataaactattgattggttgtaccttgttctttaatcaaaaattataggaacataaagataatgggtaacacagctagtcaggaacaaaatacctttacactattgccccagggaaatggagattggagagatgactaaagtcccatgctcatgtctctctgggcctgataaattttgtgttgctcatacagctcagactgccctcagctatttttctcttctcagatagatatgaaagataggagagataaatcagatacgtgagtggtagaatcttaaatattgaaagtaaatgtaaataagtctcaatctatttataattatttactagttttcacataaaagtcatttcttaatttttagctattagttctttacctttcttaatttcaaaccaaacatttacgttaacaaacatttaactatttcagacacagatttagatgataaattagcataatataaaccgatgattttgagttttaagagtattatctttccatcccaaacatttttgcacatggtattaaaacaaatgagtaatcattgcaataagtaatcattgcaagttttctctagctgtaactcttcatttgtggggccattatatattctgttgccctgagttatgtgcaagttgttaccaccagttgtcaggcaggaccaaaaatataaatgttgattaaaataaaaacagaacatttactatatagctgatataagtttatatctacaatttttgctgcctctaatctaacctaaatgcttaattgcatcaaaaagtttttaaatcttatgaaacaccattttgtaggtgccttcattatgacagagtcccaaactattgtatcaattatttgtagactattttctatcaatctttaatctggtagcaacagcaaaacattgtttttcagatgcatgcatatgtagcattctacatggctcaaaagagcctgggtcctttccctcagagagcttactgcactacttggcaaataagacatatgcccagaaagcaactagagttccactttatatagaaaataattaagggtcaaaatgatgaaagagcaactttcagaggatttcaagtaagtcaaagatgaaagttgagtagaatgcttcttagcctatgtggagctaaacgtagagtttgaagaacaggcattaatcttttctcaggcttttcacagcatactaacctctgctttagcatggtacctagaaagtctcactcaaagaagtagagagaaagatgcacatcagagactgggaaactcatacacttatttgcatttgtattgacatggtgtgtttatatttttatattccccattagcctgggcaagtcagggtttcaggggcagagactgctctcttcatctcacattttctgaactaagcattgtgtcttacacagagaagatgtctaattaatattttttaactgaaccaaattattgtggatttctatgagtgtcatttcacgaagcaatgacctaattggtataagtatgactacttgtgtatagcatccaacttgcccaataacttttataattttaaagggaaaaggatgtttgagcccacgggatggggcacttttatatttgagtttgtggttgttgcaattgtttgagtgaaaaaaaagaactctacttttcccatggcaagcaaagccagctgagggtttgtaccatccagttctgaaagttaattttgatcttcagtaaatgtcccttggcatctgcaggggattggttccaggacctctcatggataccaaagtctgagaatgctcaagtccctggtattaattgctgtagcatttgcatatttcctatgcacatcctctcctgtactttaaatcattgaaagagtacctataatatctagtacaatgtaaatgctctgtaaatagtttttgcactatattgtctagggaataatgacaaaggaaaaagagtctatacatcttcagtacagacacaatctttaaaaaaattttttgatccatggttagttgaatccacagaagtgaaacctatggatttagagggccaactatatatactaaggaaaagttcaaggaaggagtaagaaagtagttattttcaatgtaaatgatgataatgataatacctaccatttatttatcaccatatgaaaggagctttgctaagtttttcatgctttcatttaaccacgcaactctatgggatagttgttattatttccattttacagataaggaaactgactaaagagactaagtctgtagtattctgtatcttctactgtaacaacaacaacagcaaaaccatgtgagaagagagagatagaaggagcgtactgatgatattgaatgggcatcagcaatatttatcagccttactctctgtactttaatcttttaaaaagtcaattcctgtgacccaaacaatttctcatatcatcttatgcttctgtgttcctgctttatttcttctttcacactccatgttctgctggtgtggaacagatgttgcatagaaagcatgcagataggttttgtttggcccatagtgtttaaatattttgaaaatgttcataattcaagcaggaatgtacctctctaatttgctgtagacctcaccacctaccaacttatatttggtctaatttatacattcattttgtggcattggagtttgctagtcattatttatagcttaaaggatttaaactttttacttaatactagaagctgtgtgtttttcttggaaaagtatatttttcaaattgtttgacactgaagcagaaacatcactgcaaacatacatggatcaaatacatgatattctccttctataaagatatagtattatatgttatattaacactgtaaggaattaaatagtgataacctccatcacaaagtgcacatggcattcacatgaaatatgtatatgaaatcattttataaactgtgaagcaccatacaaaatgcattttttattactcttaattcctcatttgttccctttaatattggggatggaaaaacactccacaattaaaaccacctttgcaaaaactacaactgagaaaattatgactgaaagatctgacttgactccactttgcttctaacctgcaagctgttcttgttcattcatggacataggccaaaataactttgggaggaacttgaaactaagacaataatagccctttcacaaaacaaacctctttcctgcctggggactacactaccttcataggactaacaaattagccacaagattagaaataatggtttaggaatcacatagctggagactgaaagattcgaaacctccccagattgcttctggggataaatcactattgcaaaacctaagatcagtgcttgagatattttgcagatgctgtacaggatggctcagctgctaccacccagactgataaactgggtcatctggtctcctggccccaggagactgactcagcatcaagaggacaactttgacaccctatgatatcatctccaacccaaccaatcagcagtccccactttcagacccctacccaccaagttttccttaaaacccccatcctcaagtttttgagaagactgatttgagtaataataacactcttctctcctgtacagctggctgtgcgtgaattaaacttttctctattgcaa

SEQ ID NO: 164 - DNA, 2444 bp, Homo sapiens
cttgaaaacaggtctatgtagttttaacagctcttttgtgaacttcggatgtaactgaatcattttctagatttatctgttaaataaatggtccatgaaagtatgtatgggtagataattgatggaagcaaataatcctattgaaacatttgctatttatttttgatacagttcttttaaacagactgattagttccctttatgatttcaccctcgaaagcacacacgattttgttctttatcattgttgcatgacattccatttggcagttgggcttatttttagccagactatcaaagtatttttcaactggactgtcactgcacttgaacttggaatcttataacttgaagaactgccctggagaaaggaagaaacttataataaatgggaaattataaatctagaccaacccaaacttgtactgatgaatggaagaaaaaagtcagtgaatcatatgttatcacaatagaaagattagaagatgacctgcagatcaaggaaaaagaactgacagaactaaggaatatatttggctctgatgaagccttcagtaaagtcaatttaaattaccgcactgaaaatgggctgtctctacttcatttatgttgcatttgtggaggcaagaaatcacatattcgaactcttatgttgaaagggctccgcccatctcgactgacaagaaatggatttacagccttgcatttagcagtttacaaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggttcttttgggaaagtatataaaggacgatgcagaaataaaatagtggctataaaacgttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagaggattcttgatttgcagtctaaattaattattgcagtagatgttgccaaaggcatggagtaccttcacaacctgacacagccaattatacatcgtgacttgaacagatgctgtacaggatggctcagctgctaccacccagactgataaactgggtcatctggtctcctggccccaggagactgactcagcatcaagaggacaactttgacaccctatgatatcatctccaacccaaccaatcagcagtccccactttcagacccctacccaccaagttttccttaaaacccccatcctcaagtttttgagaagactgatttgagtaataataacactcttctctcctgtacagctggctgtgcgtgaattaaacttttctctattgcaa

SEQ ID NO: 165 - DNA, 2352 bp, Homo sapiens
cttgaaaacaggtctatgtagttttaacagctcttttgtgaacttcggatgtaactgaatcattttctagatttatctgttaaataaatggtccatgaaagtatgtatgggtagataattgatggaagcaaataatcctattgaaacatttgctatttatttttgatacagttcttttaaacagactgattagttccctttatgatttcaccctcgaaagcacacacgattttgttctttatcattgttgcatgacattccatttggcagttgggcttatttttagccagactatcaaagtatttttcaactggactgtcactgcacttgaacttggaatcttataacttgaagaactgccctggagaaaggaagaaacttataataaatgggaaattataaatctagaccaacccaaacttgtactgatgaatggaagaaaaaagtcagtgaatcatatgttatcacaatagaaagattagaagatgacctgcagatcaaggaaaaagaactgacagaactaaggaatatatttggctctgatgaagccttcagtaaagtcaatttaaattaccgcactgaaaatgggctgtctctacttcatttatgttgcatttgtggaggcaagaaatcacatattcgaactcttatgttgaaagggctccgcccatctcgactgacaagaaatggatttacagccttgcatttagcagtttacaaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggttcttttgggaaagtatataaaggacgatgcagaaataaaatagtggctataaaacgttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagaggattcttgatttgcagtctaaattaattattgcagtagatgttgccaaaggcatggagtaccttcacaacctgacacagccaattatacatcgtgacttgaacagggcttcctgaactatgtatgtgattgctagagctcaagcagccatctttgaacatgaggcagtgcatcagaaagagcagagcagcaagaaagaaggcatctcagttcctaacgaatgaagtcaccacatcagccatagaagcctgcctctaggactctgaaataaaaaagagaaataaactcttagtttaag

SEQ ID NO: 166 - DNA, 2339 bp, Homo sapiens
gtgctgtgcggcgcggtctcagggaaggtggggctatggcagctgctagggaccctccggaagtatcgctgcgagaagccacccagcgaaaattgcggaggttttccgagctaagaggcaaacttgtagcacgtggagaattctgggacatagttgcaataacagcggctgatgaaaaacaggaacttgcttacaaccaacagctgtcagaaaagctgaaaagaaaggagttaccccttggagttcaatatcacgtttttgtagatcctgctggagccaaaattggaaatggaggatcaacactttgtgcccttcaatgtttggaaaagctatatggagataaatggaattcttttaccatcttattaattcactctgatgaatggaagaaaaaagtcagtgaatcatatgttatcacaatagaaagattagaagatgacctgcagatcaaggaaaaagaactgacagaactaaggaatatatttggctctgatgaagccttcagtaaagtcaatttaaattaccgcactgaaaatgggctgtctctacttcatttatgttgcatttgtggaggcaagaaatcacatattcgaactcttatgttgaaagggctccgcccatctcgactgacaagaaatggatttacagccttgcatttagcagtttacaaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggttcttttgggaaagtatataaaggacgatgcagaaataaaatagtggctataaaacgttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagaggtatgggtcttttgttctgatttatccttggacattccgtagaaactactcttgcaatacttcagagggtttcccattggatgagccttcaccttttgaaatctgagtgctgctagcaagtgtgtcatttttaatatcgctaataatttatgcctaataactgtgtttcaatgaatagaaatactttatgctttcttagcagaaattcagttgaagataacaaatttaaggaatttaaattggaaactggagcttaaaataattaggctaaatttgaaatactgataaagaagatatatgtaagctcagaataaaataaacttttaaaaaagtg

SEQ ID NO: 167 - DNA, 2389 bp, Homo sapiens
cttgaaaacaggtctatgtagttttaacagctcttttgtgaacttcggatgtaactgaatcattttctagatttatctgttaaataaatggtccatgaaagtatgtatgggtagataattgatggaagcaaataatcctattgaaacatttgctatttatttttgatacagttcttttaaacagactgattagttccctttatgatttcaccctcgaaagcacacacgattttgttctttatcattgttgcatgacattccatttggcagttgggcttatttttagccagactatcaaagtatttttcaactggactgtcactgcacttgaacttggaatcttataacttgaagaactgccctggagaaaggaagaaacttataataaatgggaaattataaatctagaccaacccaaacttgtactgatgaatggaagaaaaaagtcagtgaatcatatgttatcacaatagaaagattagaagatgacctgcagatcaaggaaaaagaactgacagaactaaggaatatatttggctctgatgaagccttcagtaaagtcaatttaaattaccgcactgaaaatgggctgtctctacttcatttatgttgcatttgtggaggcaagaaatcacatattcgaactcttatgttgaaagggctccgcccatctcgactgacaagaaatggatttacagccttgcatttagcagtttacaaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggttcttttgggaaagtatataaaggacgatgcagaaataaaatagtggctataaaacgttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagaggtatgggtcttttgttctgatttatccttggacattccgtagaaactactcttgcaatacttcagagggtttcccattggatgagccttcaccttttgaaatctgagtgctgctagcaagtgtgtcatttttaatatcgctaataatttatgcctaataactgtgtttcaatgaatagaaatactttatgctttcttagcagaaattcagttgaagataacaaatttaaggaatttaaattggaaactggagcttaaaataattaggctaaatttgaaatactgataaagaagatatatgtaagctcagaataaaataaacttttaaaaaagtg

SEQ ID NO: 168 - DNA, 1829 bp, Homo sapiens
cttgaaaacaggtctatgtagttttaacagctcttttgtgaacttcggatgtaactgaatcattttctagatttatctgttaaataaatggtccatgaaagtatgtatgggtagataattgatggaagcaaataatcctattgaaacatttgctatttatttttgatacagttcttttaaacagactgattagttccctttatgatttcaccctcgaaagcacacacgattttgttctttatcattgttgcatgacattccatttggcagttgggcttatttttagccagactatcaaagtatttttcaactggactgtcactgcacttgaacttggaatcttataacttgaagaactgccctggagaaaggaagaaacttataataaatgggaaattataaatctagaccaacccaaacttgtactgatgaatggaagaaaaaagtcagtgaatcatatgttatcacaatagaaagattagaagatgacctgcagatcaaggaaaaagaactgacagaactaaggaatatatttggctctgatgaagccttcagtaaagtcaatttaaattaccgcactgaaaatgggctgtctctacttcatttatgttgcatttgtggaggcaagaaatcacatattcgaactcttatgttgaaagggctccgcccatctcgactgacaagaaatggatttacagccttgcatttagcagtttacaaggataatgcagaattgatcacttctctgcttcacagtggagctgatatacagcaggttggatacggtggcctcactgccctccatattgctacaatagctggccacctagaggctgctgatgtgctgttgcaacatggagctaatgtcaatattcaagatgcagtttttttcactccattgcatattgcagcgtactatggacatgaacaggtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatgtgaatgctcaagataatgaagaccatgtcccactccatttctgttctcgatttggacaccatgatatagttaagtatctgctgcaaagtgatttggaagttcaacctcatgttgttaatatctatggagatacccccttacacctggcatgctacaatggcaaatttgaagttgccaaggaaatcatccaaatatcaggaacagaaagtctgactaaggaaaacatcttcagtgaaacagcttttcatagtgcttgtacctatggcaagagcattgacctagtcaaatttcttcttgatcagaatgtcataaacatcaaccaccaaggaagggatgggcacactggattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaagggcatgatgccattgtcacactcctgaagcattataagagaccacaagatgaattgccctgtaatgaatattctcagcctggaggagatggctcctatgtgtctgttccatcacccttggggaagattaaaagcatgacaaaagagaaggcagatattctcctcctaagagctggattgccttcacatttccatcttcagctctcagaaattgagttccatgagattattggctcaggtaacctaaaataaataaataaataaa

SEQ ID NO: 169 - DNA, 1671 bp, Homo sapiens
acctgatccagccagtgataaatctacttcataggaatattctaggaactcctcgggagccatttctctcctcagccatgagcacactatttatgggcctaatttagtaacttcctaaattacaacctgaagacttgaaggaggtgcctcccacagtgtgtgattcactcccagggagacatagggtccctggatgaatccatggttggctattctgaaccttgtaaggcttctaagggaggaagatgtttatagatttaaaaaaataatgctgcttctcattgtataagaggcagacttacttgatgattaattacttgtgtgatgttttaggaagaattatgaactttatgatcttccaatgattaaaaaatgccctttgaggaactgaacagccacagtaaacacactgtgtatgtataaacatgtttattgcacacactgagggcaatatgtatggatgttaatttatatttatgccttttgagagcatcgggagaacacagtaaattctcactaagaagaatgctactctgcagtggaagaaccaaacacttggtaaatggcttgtggatgtttcttgatgtgcagaacctccgttggatggctcctgaggtgttcacgcagtgcactcggtacaccatcaaagcagatgtcttcagctatgctctgtgtctgtgggaaattctcactggcgaaattccattcgctcatctcaagccagcggctgcggcagcagacatggcttaccaccacatcagacctcccattggctattccattcccaagcccatatcatctctgctgatacgagggtggaacgcatgtcctgaaggaagacccgaattttctgaagttgtcatgaagttagaagagtgtctctgcaacattgagctgatgtctcctgcatcaagtaacagcagtgggtctctctcaccttcttcttcttctgattgcctggtgaaccggggaggacctggccggagtcatgtggcagcattaagaagtcgtttcgaattggaatatgctctaaatgcaaggtcctatgctgctttgtcccaaagtgctggacaatattcctctcaaggtctgtctttggaggagatgaaaagaagtcttcaatacacacccattgacaaatatggctatgtatccgatcccatgagctcaatgcattttcattcttgccgaaatagtagcagctttgaggacagcagctgacagcattcggcgtatacctaaggagagttttttccccgaactgacagcaacgattccaaccacggcaagctggcttccaactataacattttactctcaaaggtctccttaaattgggcttgtttttacttgtcctatttaattccccactattagcaggctttggatttgtgcctaaggaataatatgcaaaagaaccaagacagaatgtatatgaagaattgtttttaattttgtaaattaaaaaaaaatttagatcgttacttggaaatggagcctaagtctgtggtggacagataataattatgttttcctgggctgaattatgtagacttgtgtttgacagctatgggtttatttcttagaacattgttcattttcttttctcattatgttacttctagtgttcacctctgtgattaaagattctttggtgaaatagaaaattga

SEQ ID NO: 170 - DNA, 1745 bp, Homo sapiens
acctgatccagccagtgataaatctacttcataggaatattctaggaactcctcgggagccatttctctcctcagccatgagcacactatttatgggcctaatttagtaacttcctaaattacaacctgaagacttgaaggaggtgcctcccacagtgtgtgattcactcccagggagacatagggtccctggatgaatccatggttggctattctgaaccttgtaaggcttctaagggaggaagatgtttatagatttaaaaaaataatgctgcttctcattgtataagaggcagacttacttgatgattaattacttgtgtgatgttttaggaagaattatgaactttatgatcttccaatgattaaaaaatgccctttgaggaactgaacagccacagtaaacacactgtgtatgtataaacatgtttattgcacacactgagggcaatatgtatggatgttaatttatatttatgccttttgagagcatcgggagaacacagtaaattctcactaagaagaatgctactctgcagtggaagaaccaaacacttggtaaatggcttgtggatgtttcttgatgtgcagaacctccgttggatggctcctgaggtgttcacgcagtgcactcggtacaccatcaaagcagatgtcttcagctatgctctgtgtctgtgggaaattctcactggcgaaattccattcgctcatctcaagccagcggctgcggcagcagacatggcttaccaccacatcagacctcccattggctattccattcccaagcccatatcatctctgctgatacgagggtggaacgcatgtcctgaaggaagacccgaattttctgaagttgtcatgaagttagaagagtgtctctgcaacattgagctgatgtctcctgcatcaagtaacagcagtgggtctctctcaccttcttcttcttctgattgcctggtgaaccggggaggacctggccggagtcatgtggcagcattaagaagtcgtttcgaattggaatatgctctaaatgcaaggtcctatgctgctttgtcccaaagtgctggacaatattcctctcaaggtctgtctttggaggagatgaaaagaagtcttcaatacacacccattgacaaatatgatgtaacttcataaacacaaaacatagggcatcctttgaaacatttgctttgaccagaagtcttcccttttgtggctatgtatccgatcccatgagctcaatgcattttcattcttgccgaaatagtagcagctttgaggacagcagctgacagcattcggcgtatacctaaggagagttttttccccgaactgacagcaacgattccaaccacggcaagctggcttccaactataacattttactctcaaaggtctccttaaattgggcttgtttttacttgtcctatttaattccccactattagcaggctttggatttgtgcctaaggaataatatgcaaaagaaccaagacagaatgtatatgaagaattgtttttaattttgtaaattaaaaaaaaatttagatcgttacttggaaatggagcctaagtctgtggtggacagataataattatgttttcctgggctgaattatgtagacttgtgtttgacagctatgggtttatttcttagaacattgttcattttcttttctcattatgttacttctagtgttcacctctgtgattaaagattctttggtgaaatagaaaattga

SEQ ID NO: 171 - DNA, 168 bp, Homo sapiens
gcaaacttgtagcacgtggagaattctgggacatagttgcaataacagcggctgatgaaaaacaggaacttgcttacaaccaacagctgtcagaaaagctgaaaagaaaggagttaccccttggagttcaatatcacgtttttgtagatcctgctggagccaaaattg

SEQ ID NO: 172 - DNA, 428 bp, Homo sapiens
cttgaaaacaggtctatgtagttttaacagctcttttgtgaacttcggatgtaactgaatcattttctagatttatctgttaaataaatggtccatgaaagtatgtatgggtagataattgatggaagcaaataatcctattgaaacatttgctatttatttttgatacagttcttttaaacagactgattagttccctttatgatttcaccctcgaaagcacacacgattttgttctttatcattgttgcatgacattccatttggcagttgggcttatttttagccagactatcaaagtatttttcaactggactgtcactgcacttgaacttggaatcttataacttgaagaactgccctggagaaaggaagaaacttataataaatgggaaattataaatctagaccaacccaaacttgtactg

SEQ ID NO: 173 - DNA, 139 bp, Homo sapiens
gtaactcgccttcttttgaaatttggtgctgatgtaaatgtaagtggtgaagttggagatagacccctccacctagcatctgcaaaaggattcttgaatattgcaaaactcttgatggaagaaggcagcaaagcagatg

SEQ ID NO: 174 - DNA, 145 bp, Homo sapiens
tgaatgctcaagataatgaagaccatgtcccactccatttctgttctcgatttggacaccatgatatagttaagtatctgctgcaaagtgatttggaagttcaacctcatgttgttaatatctatggagatacccccttacacct

SEQ ID NO: 175 - DNA, 150 bp, Homo sapiens
gattacactctgcttgctaccacggtcacattcgcctggttcagttcttactggataatggagctgatatgaatctagtggcttgtgatcccagcaggtctagtggtgaaaaagatgagcagacatgtttgatgtgggcttatgaaaaag

SEQ ID NO: 176 - DNA, 195 bp, Homo sapiens
ttatcgagccaatacctactgctccaagtcagatgtggatatgttttgccgagaggtgtccattctctgccagctcaatcatccctgcgtaattcagtttgtgggtgcttgcttgaatgatcccagccagtttgccattgtcactcaatacatatcagggggttctctgttctccctccttcatgagcagaagag

SEQ ID NO: 177 - DNA, 334 bp, Homo sapiens
gtatgggtcttttgttctgatttatccttggacattccgtagaaactactcttgcaatacttcagagggtttcccattggatgagccttcaccttttgaaatctgagtgctgctagcaagtgtgtcatttttaatatcgctaataatttatgcctaataactgtgtttcaatgaatagaaatactttatgctttcttagcagaaattcagttgaagataacaaatttaaggaatttaaattggaaactggagcttaaaataattaggctaaatttgaaatactgataaagaagatatatgtaagctcagaataaaataaacttttaaaaaagtg

SEQ ID NO: 178 - DNA, 5172 bp, Homo sapiens
gtatttttttcctaaataatgaactcagaagggtatgactaactgggagtttaagacagatttcagtgaagatacattttagacttattgcagatcagggtactctgtggttaagaggacaggctaccataagcttaggcgatagaaagttttgtcattgtcttttaaggagtagaaagctgtataactgcttctcagggatcttttaaatctatgaagaatattcacaaatatagtaatatctgtaagatgcaccagctaactagttcccaggtcagtaaggtcacatcaagcagtgagctaaatggctattaaaaaaatgatatgtctcctataaagtgaagtgttggagtagctcctgatataaaattatctgaaacgtactttgtgctaccgtaaattcagcttaaccacaacagcttatttacctagttatcaggtaaagaggatttcatgtgaaacaaatataaaatcagagtgttccagtggtggaaccccaagtgacctcaaaacttttcctaaatagtgaaaactttctctgagtaaagtcttataggaaaggccaatatacaaaatggagcagtgttgggtgaaggtaaagtggggaggagaaaactctatttacttcccatttcccactgctaaggagagagattcctgcaccacctctgtggaagcccaaaggtgctgtagagaatagttggacaaacactgatttagtttatgaagcaagtcagattattccacatcaatcactttcattttatgatagtcaataagattcaaatagattgtgtatatagtcctttatagaagctttacagttctgaagctagtaaatattgattactctagtcagtcatttggtcaacaactatgtttgaacatatatgtttaaagactattttcagcacagtagagcttacaaagtgggacgttcatagactacattcagggagttttcacactctatgtagaggagaaaaatatttacctaaatgtatttgaggtataaaataataagttatatacaagatttagtacaaaaatttcaggaacaagaatattaattgcttctgaggattctaacaacaactctatggaagggatgacatatgagcttggtcctgaaagatgggtagtttggaagagcatcctataggcttagggaagacataataaaatgcatagagattagaaagttaaaggtatatataggaactatagtttcattttagtatacaaagtgtataaatgtatgtaatgtgtataatgtaagaagtggacaactggaaaaaggtatagaaaaatagaaaaagagcacagacaccagtgaatgctattactaaggtatttcagattttttttcctgtaaggagaccataaagaggtttgagctgaaaatcacaaatttggagctatattattgaaagatgaatccagcatctggtaaaatatcaacttgaagggaaagtcaagaaactagtgagggagttgttgcagtaatctagataagaagtaacgaaaagcagaatttcaccggtggcagtggatatgaaaggaagagacatgtcaactttaaataacaaaatacagagtttattcaagctcaaagcttgaggatcattacccatgagcatggactcaagttgtcctgaatacatactccaattagcagtggttacaagatagtttttaagaaaaaaaaaaaaaaaaaaaaaagcagtttttaagttgtttacaaagaatttacataaataatataaactattgattggttgtaccttgttctttaatcaaaaattataggaacataaagataatgggtaacacagctagtcaggaacaaaatacctttacactattgccccagggaaatggagattggagagatgactaaagtcccatgctcatgtctctctgggcctgataaattttgtgttgctcatacagctcagactgccctcagctatttttctcttctcagatagatatgaaagataggagagataaatcagatacgtgagtggtagaatcttaaatattgaaagtaaatgtaaataagtctcaatctatttataattatttactagttttcacataaaagtcatttcttaatttttagctattagttctttacctttcttaatttcaaaccaaacatttacgttaacaaacatttaactatttcagacacagatttagatgataaattagcataatataaaccgatgattttgagttttaagagtattatctttccatcccaaacatttttgcacatggtattaaaacaaatgagtaatcattgcaataagtaatcattgcaagttttctctagctgtaactcttcatttgtggggccattatatattctgttgccctgagttatgtgcaagttgttaccaccagttgtcaggcaggaccaaaaatataaatgttgattaaaataaaaacagaacatttactatatagctgatataagtttatatctacaatttttgctgcctctaatctaacctaaatgcttaattgcatcaaaaagtttttaaatcttatgaaacaccattttgtaggtgccttcattatgacagagtcccaaactattgtatcaattatttgtagactattttctatcaatctttaatctggtagcaacagcaaaacattgtttttcagatgcatgcatatgtagcattctacatggctcaaaagagcctgggtcctttccctcagagagcttactgcactacttggcaaataagacatatgcccagaaagcaactagagttccactttatatagaaaataattaagggtcaaaatgatgaaagagcaactttcagaggatttcaagtaagtcaaagatgaaagttgagtagaatgcttcttagcctatgtggagctaaacgtagagtttgaagaacaggcattaatcttttctcaggcttttcacagcatactaacctctgctttagcatggtacctagaaagtctcactcaaagaagtagagagaaagatgcacatcagagactgggaaactcatacacttatttgcatttgtattgacatggtgtgtttatatttttatattccccattagcctgggcaagtcagggtttcaggggcagagactgctctcttcatctcacattttctgaactaagcattgtgtcttacacagagaagatgtctaattaatattttttaactgaaccaaattattgtggatttctatgagtgtcatttcacgaagcaatgacctaattggtataagtatgactacttgtgtatagcatccaacttgcccaataacttttataattttaaagggaaaaggatgtttgagcccacgggatggggcacttttatatttgagtttgtggttgttgcaattgtttgagtgaaaaaaaagaactctacttttcccatggcaagcaaagccagctgagggtttgtaccatccagttctgaaagttaattttgatcttcagtaaatgtcccttggcatctgcaggggattggttccaggacctctcatggataccaaagtctgagaatgctcaagtccctggtattaattgctgtagcatttgcatatttcctatgcacatcctctcctgtactttaaatcattgaaagagtacctataatatctagtacaatgtaaatgctctgtaaatagtttttgcactatattgtctagggaataatgacaaaggaaaaagagtctatacatcttcagtacagacacaatctttaaaaaaattttttgatccatggttagttgaatccacagaagtgaaacctatggatttagagggccaactatatatactaaggaaaagttcaaggaaggagtaagaaagtagttattttcaatgtaaatgatgataatgataatacctaccatttatttatcaccatatgaaaggagctttgctaagtttttcatgctttcatttaaccacgcaactctatgggatagttgttattatttccattttacagataaggaaactgactaaagagactaagtctgtagtattctgtatcttctactgtaacaacaacaacagcaaaaccatgtgagaagagagagatagaaggagcgtactgatgatattgaatgggcatcagcaatatttatcagccttactctctgtactttaatcttttaaaaagtcaattcctgtgacccaaacaatttctcatatcatcttatgcttctgtgttcctgctttatttcttctttcacactccatgttctgctggtgtggaacagatgttgcatagaaagcatgcagataggttttgtttggcccatagtgtttaaatattttgaaaatgttcataattcaagcaggaatgtacctctctaatttgctgtagacctcaccacctaccaacttatatttggtctaatttatacattcattttgtggcattggagtttgctagtcattatttatagcttaaaggatttaaactttttacttaatactagaagctgtgtgtttttcttggaaaagtatatttttcaaattgtttgacactgaagcagaaacatcactgcaaacatacatggatcaaatacatgatattctccttctataaagatatagtattatatgttatattaacactgtaaggaattaaatagtgataacctccatcacaaagtgcacatggcattcacatgaaatatgtatatgaaatcattttataaactgtgaagcaccatacaaaatgcattttttattactcttaattcctcatttgttccctttaatattggggatggaaaaacactccacaattaaaaccacctttgcaaaaactacaactgagaaaattatgactgaaagatctgacttgactccactttgcttctaacctgcaagctgttcttgttcattcatggacataggccaaaataactttgggaggaacttgaaactaagacaataatagccctttcacaaaacaaacctctttcctgcctggggactacactaccttcataggactaacaaattagccacaagattagaaataatggtttaggaatcacatagctggagactgaaagattcgaaacctccccagattgcttctggggataaatcactattgcaaaacctaagatcagtgcttgagatattttgcag

SEQ ID NO: 179 - DNA, 284 bp, Homo sapiens
atgctgtacaggatggctcagctgctaccacccagactgataaactgggtcatctggtctcctggccccaggagactgactcagcatcaagaggacaactttgacaccctatgatatcatctccaacccaaccaatcagcagtccccactttcagacccctacccaccaagttttccttaaaacccccatcctcaagtttttgagaagactgatttgagtaataataacactcttctctcctgtacagctggctgtgcgtgaattaaacttttctctattgcaa

SEQ ID NO: 180 - DNA, 192 bp, Homo sapiens
ggcttcctgaactatgtatgtgattgctagagctcaagcagccatctttgaacatgaggcagtgcatcagaaagagcagagcagcaagaaagaaggcatctcagttcctaacgaatgaagtcaccacatcagccatagaagcctgcctctaggactctgaaataaaaaagagaaataaactcttagtttaag

SEQ ID NO: 181 - DNA, 591 bp, Homo sapiens
acctgatccagccagtgataaatctacttcataggaatattctaggaactcctcgggagccatttctctcctcagccatgagcacactatttatgggcctaatttagtaacttcctaaattacaacctgaagacttgaaggaggtgcctcccacagtgtgtgattcactcccagggagacatagggtccctggatgaatccatggttggctattctgaaccttgtaaggcttctaagggaggaagatgtttatagatttaaaaaaataatgctgcttctcattgtataagaggcagacttacttgatgattaattacttgtgtgatgttttaggaagaattatgaactttatgatcttccaatgattaaaaaatgccctttgaggaactgaacagccacagtaaacacactgtgtatgtataaacatgtttattgcacacactgagggcaatatgtatggatgttaatttatatttatgccttttgagagcatcgggagaacacagtaaattctcactaagaagaatgctactctgcagtggaagaaccaaacacttggtaaatggcttgtggatgtttcttgatgtgcag

SEQ ID NO: 182 - DNA, 133 bp, Homo sapiens
aacctccgttggatggctcctgaggtgttcacgcagtgcactcggtacaccatcaaagcagatgtcttcagctatgctctgtgtctgtgggaaattctcactggcgaaattccattcgctcatctcaagccag

SEQ ID NO: 183 - DNA, 291 bp, Homo sapiens
gcaaaatccagaccaagtcattacccagtctcatctgtgtacacagaaactcttaagaagaaaaatgaagatcgttttgggatgtggattgagtatctcagaagataacctcttatcctggccattcaacctgatgtgttacatgtttatttgtttagaatcttccatcactaccaaaatgttagctccatgaaagcggtcctttttgtttattttgttcactgctataacactaacatctaaagctggatgcttaagaatgtttgttgagtaaatgaatgaataccagta

SEQ ID NO: 184 - DNA, 170 bp, Homo sapiens
ctgatgtctcctgcatcaagtaacagcagtgggtctctctcaccttcttcttcttctgattgcctggtgaaccggggaggacctggccggagtcatgtggcagcattaagaagtcgtttcgaattggaatatgctctaaatgcaaggtcctatgctgctttgtcccaaag

SEQ ID NO: 185 - DNA, 527 bp, Homo sapiens
gctatgtatccgatcccatgagctcaatgcattttcattcttgccgaaatagtagcagctttgaggacagcagctgacagcattcggcgtatacctaaggagagttttttccccgaactgacagcaacgattccaaccacggcaagctggcttccaactataacattttactctcaaaggtctccttaaattgggcttgtttttacttgtcctatttaattccccactattagcaggctttggatttgtgcctaaggaataatatgcaaaagaaccaagacagaatgtatatgaagaattgtttttaattttgtaaattaaaaaaaaatttagatcgttacttggaaatggagcctaagtctgtggtggacagataataattatgttttcctgggctgaattatgtagacttgtgtttgacagctatgggtttatttcttagaacattgttcattttcttttctcattatgttacttctagtgttcacctctgtgattaaagattctttggtgaaatagaaaattga

SEQ ID NO: 186 - DNA, 117 bp, Homo sapiens
gtgctgtgcggcgcggtctcagggaaggtggggctatggcagctgctagggaccctccggaagtatcgctgcgagaagccacccagcgaaaattgcggaggttttccgagctaagag

SEQ ID NO: 187 - DNA, 93 bp, Homo sapiens
gaaatggaggatcaacactttgtgcccttcaatgtttggaaaagctatatggagataaatggaattcttttaccatcttattaattcactctg

SEQ ID NO: 188 - DNA, 109 bp, Homo sapiens
atgaatggaagaaaaaagtcagtgaatcatatgttatcacaatagaaagattagaagatgacctgcagatcaaggaaaaagaactgacagaactaaggaatatatttgg

SEQ ID NO: 189 - DNA, 86 bp, Homo sapiens
ctctgatgaagccttcagtaaagtcaatttaaattaccgcactgaaaatgggctgtctctacttcatttatgttgcatttgtggag

SEQ ID NO: 190 - DNA, 98 bp, Homo sapiens
gcaagaaatcacatattcgaactcttatgttgaaagggctccgcccatctcgactgacaagaaatggatttacagccttgcatttagcagtttacaag

SEQ ID NO: 191 - DNA, 45 bp, Homo sapiens
ccttctccatgtggcctgcattttcttataccatggctgacacag

SEQ ID NO: 192 - DNA, 111 bp, Homo sapiens
gataatgcagaattgatcacttctctgcttcacagtggagctgatatacagcaggttggatacggtggcctcactgccctccatattgctacaatagctggccacctagag

SEQ ID NO: 193 - DNA, 99 bp, Homo sapiens
gctgctgatgtgctgttgcaacatggagctaatgtcaatattcaagatgcagtttttttcactccattgcatattgcagcgtactatggacatgaacag

SEQ ID NO: 194 - DNA, 105 bp, Homo sapiens
ggcatgctacaatggcaaatttgaagttgccaaggaaatcatccaaatatcaggaacagaaagtctgactaaggaaaacatcttcagtgaaacagcttttcatag

SEQ ID NO: 195 - DNA, 95 bp, Homo sapiens
tgcttgtacctatggcaagagcattgacctagtcaaatttcttcttgatcagaatgtcataaacatcaaccaccaaggaagggatgggcacactg

SEQ ID NO: 196 - DNA, 87 bp, Homo sapiens
ggcatgatgccattgtcacactcctgaagcattataagagaccacaagatgaattgccctgtaatgaatattctcagcctggaggag

SEQ ID NO: 197 - DNA, 57 bp, Homo sapiens
atggctcctatgtgtctgttccatcacccttggggaagattaaaagcatgacaaaag

SEQ ID NO: 198 - DNA, 93 bp, Homo sapiens
agaaggcagatattctcctcctaagagctggattgccttcacatttccatcttcagctctcagaaattgagttccatgagattattggctcag

SEQ ID NO: 199 - DNA, 27 bp, Homo sapiens
gtaacctaaaataaataaataaataaa

SEQ ID NO: 200 - DNA, 58 bp, Homo sapiens
gttcttttgggaaagtatataaaggacgatgcagaaataaaatagtggctataaaacg

SEQ ID NO: 201 - DNA, 105 bp, Homo sapiens
gattcttgatttgcagtctaaattaattattgcagtagatgttgccaaaggcatggagtaccttcacaacctgacacagccaattatacatcgtgacttgaacag

SEQ ID NO: 202 - DNA, 119 bp, Homo sapiens
gtctgccattacctctaggatctggatcacccatagtatttgcatctggaggggagctcattactttaacagggaagaatgcaatttcaggtgtatgcttacttctgccatcctaaaag

SEQ ID NO: 203 - DNA, 53 bp, Homo sapiens
tcacaatattcttctctatgaggatgggcatgctgtggtggcagattttggag

SEQ ID NO: 204 - DNA, 53 bp, Homo sapiens
aatcaagatttctacagtctctggatgaagacaacatgacaaaacaacctggg

SEQ ID NO: 205 - DNA, 110 bp, Homo sapiens
cggctgcggcagcagacatggcttaccaccacatcagacctcccattggctattccattcccaagcccatatcatctctgctgatacgagggtggaacgcatgtcctgaa

SEQ ID NO: 206 - DNA, 60 bp, Homo sapiens
ggaagacccgaattttctgaagttgtcatgaagttagaagagtgtctctgcaacattgag

SEQ ID NO: 207 - DNA, 80 bp, Homo sapiens
tgctggacaatattcctctcaaggtctgtctttggaggagatgaaaagaagtcttcaatacacacccattgacaaatatg

SEQ ID NO: 208 - DNA, 74 bp, Homo sapiens
atgtaacttcataaacacaaaacatagggcatcctttgaaacatttgctttgaccagaagtcttcccttttgtg

SEQ ID NO: 209 - protein, 936 aa, Homo sapiens
MAAARDPPEVSLREATQRKLRRFSELRGKLVARGEFWDIVAITAADEKQELAYNQQLSEKLKRKELPLGVQYHVFVDPAGAKIGNGGSTLCALQCLEKLYGDKWNSFTILLIHSDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNSHNILLYEDGHAVVADFGESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYGYVSDPMSSMHFHSCRNSSSFEDSS

SEQ ID NO: 210 - protein, 835 aa, Homo sapiens
MGNYKSRPTQTCTDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNSHNILLYEDGHAVVADFGESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYGYVSDPMSSMHFHSCRNSSSFEDSS

SEQ ID NO: 211 - protein, 714 aa, Homo sapiens
SSGINAEWPLWPGEGGAMAAARDPPEVSLREATQRKLRRFSELRGKLVARGEFWDIVAITAADEKQELAYNQQLSEKLKRKELPLGVQYHVFVDPAGAKIGNGGSTLCALQCLEKLYGDKWNSFTILLIHSDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDMVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMSLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNRYFFPK

SEQ ID NO: 212 - protein, 969 aa, Homo sapiens
AVRRGLREGGAMAAARDPPEVSLREATQRKLRRFSELRGKLVARGEFWDIVAITAADEKQELAYNQQLSEKLKRKELPLGVQYHVFVDPAGAKIGNGGSTLCALQCLEKLYGDKWNSFTILLIHSDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNRSAITSRIWITHSICIWRGAHYFNREECNFRCMLTSAILKESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYGYVSDPMSSMHFHSCRNSSSFEDSS

SEQ ID NO: 213 - protein, 857 aa, Homo sapiens
MGNYKSRPTQTCTDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNRSAITSRIWITHSICIWRGAHYFNREECNFRCMLTSAILKESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYGYVSDPMSSMHFHSCRNSSSFEDSS

SEQ ID NO: 214 - protein, 814 aa, Homo sapiens
MGNYKSRPTQTCTDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNSHNILLYEDGHAVVADFGESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYDVTS

SEQ ID NO: 215 - protein, 739 aa, Homo sapiens
PSPCGLHFLIPWLTQDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNSHNILLYEDGHAVVADFGESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYGYVSDPMSSMHFHSCRNSSSFEDSS

SEQ ID NO: 216 - protein, 761 aa, Homo sapiens
PSPCGLHFLIPWLTQDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNRSAITSRIWITHSICIWRGAHYFNREECNFRCMLTSAILKESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYGYVSDPMSSMHFHSCRNSSSFEDSS

SEQ ID NO: 217 - protein, 854 aa, Homo sapiens
AVRRGLREGGAMAAARDPPEVSLREATQRKLRRFSELRGKLVARGEFWDIVAITAADEKQELAYNQQLSEKLKRKELPLGVQYHVFVDPAGAKIGNGGSTLCALQCLEKLYGDKWNSFTILLIHSDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNSHNILLYEDGHAVVADFGESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEAKSRPSHYPVSSVYTETLKKKNEDRFGMWIEYLRR

SEQ ID NO: 218 - protein, 742 aa, Homo sapiens
MGNYKSRPTQTCTDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNSHNILLYEDGHAVVADFGESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEAKSRPSHYPVSSVYTETLKKKNEDRFGMWIEYLRR

SEQ ID NO: 219 - protein, 708 aa, Homo sapiens
AVRRGLREGGAMAAARDPPEVSLREATQRKLRRFSELRGKLVARGEFWDIVAITAADEKQELAYNQQLSEKLKRKELPLGVQYHVFVDPAGAKIGNGGSTLCALQCLEKLYGDKWNSFTILLIHSDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNRYFFPK

SEQ ID NO: 220 - protein, 603 aa, Homo sapiens
MGNYKSRPTQTCTDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNRCCTGWLSCYHPD

SEQ ID NO: 221 - protein, 593 aa, Homo sapiens
MGNYKSRPTQTCTDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNRAS

SEQ ID NO: 222 - protein, 702 aa, Homo sapiens
AVRRGLREGGAMAAARDPPEVSLREATQRKLRRFSELRGKLVARGEFWDIVAITAADEKQELAYNQQLSEKLKRKELPLGVQYHVFVDPAGAKIGNGGSTLCALQCLEKLYGDKWNSFTILLIHSDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRYGSFVLIYPWTFRRNYSCNTSEGFPLDEPSPFEI

SEQ ID NO: 223 - protein, 590 aa, Homo sapiens
MGNYKSRPTQTCTDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRYGSFVLIYPWTFRRNYSCNTSEGFPLDEPSPFEI

SEQ ID NO: 224 - protein, 475 aa, Homo sapiens
MGNYKSRPTQTCTDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGNLK

SEQ ID NO: 225 - protein, 254 aa, Homo sapiens
MDVNLYLCLLRASGEHSKFSLRRMLLCSGRTKHLVNGLWMFLDVQNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYGYVSDPMSSMHFHSCRNSSSFEDSS

SEQ ID NO: 226 - protein, 233 aa, Homo sapiens
MDVNLYLCLLRASGEHSKFSLRRMLLCSGRTKHLVNGLWMFLDVQNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYDVTS

SEQ ID NO: 227 - protein, 958 aa, Homo sapiens
MAAARDPPEVSLREATQRKLRRFSELRGKLVARGEFWDIVAITAADEKQELAYNQQLSEKLKRKELPLGVQYHVFVDPAGAKIGNGGSTLCALQCLEKLYGDKWNSFTILLIHSDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNRSAITSRIWITHSICIWRGAHYFNREECNFRCMLTSAILKESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYGYVSDPMSSMHFHSCRNSSSFEDSS

SEQ ID NO: 228 - protein, 843 aa, Homo sapiens
MAAARDPPEVSLREATQRKLRRFSELRGKLVARGEFWDIVAITAADEKQELAYNQQLSEKLKRKELPLGVQYHVFVDPAGAKIGNGGSTLCALQCLEKLYGDKWNSFTILLIHSDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNSHNILLYEDGHAVVADFGESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEAKSRPSHYPVSSVYTETLKKKNEDRFGMWIEYLRR

SEQ ID NO: 229 - protein, 697 aa, Homo sapiens
MAAARDPPEVSLREATQRKLRRFSELRGKLVARGEFWDIVAITAADEKQELAYNQQLSEKLKRKELPLGVQYHVFVDPAGAKIGNGGSTLCALQCLEKLYGDKWNSFTILLIHSDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRILDLQSKLIIAVDVAKGMEYLHNLTQPIIHRDLNRYFFPK

SEQ ID NO: 230 - protein, 691 aa, Homo sapiens
MAAARDPPEVSLREATQRKLRRFSELRGKLVARGEFWDIVAITAADEKQELAYNQQLSEKLKRKELPLGVQYHVFVDPAGAKIGNGGSTLCALQCLEKLYGDKWNSFTILLIHSDEWKKKVSESYVITIERLEDDLQIKEKELTELRNIFGSDEAFSKVNLNYRTENGLSLLHLCCICGGKKSHIRTLMLKGLRPSRLTRNGFTALHLAVYKDNAELITSLLHSGADIQQVGYGGLTALHIATIAGHLEAADVLLQHGANVNIQDAVFFTPLHIAAYYGHEQVTRLLLKFGADVNVSGEVGDRPLHLASAKGFLNIAKLLMEEGSKADVNAQDNEDHVPLHFCSRFGHHDIVKYLLQSDLEVQPHVVNIYGDTPLHLACYNGKFEVAKEIIQISGTESLTKENIFSETAFHSACTYGKSIDLVKFLLDQNVININHQGRDGHTGLHSACYHGHIRLVQFLLDNGADMNLVACDPSRSSGEKDEQTCLMWAYEKGHDAIVTLLKHYKRPQDELPCNEYSQPGGDGSYVSVPSPLGKIKSMTKEKADILLLRAGLPSHFHLQLSEIEFHEIIGSGSFGKVYKGRCRNKIVAIKRYRANTYCSKSDVDMFCREVSILCQLNHPCVIQFVGACLNDPSQFAIVTQYISGGSLFSLLHEQKRYGSFVLIYPWTFRRNYSCNTSEGFPLDEPSPFEI

SEQ ID NO: 231 - DNA, 23 bp, Artificial Sequence
  Synthetic oligonucleotide
gagccaatacctactgctccaag

SEQ ID NO: 232 - DNA, 23 bp, Artificial Sequence
  Synthetic oligonucleotide
gcaagcacccacaaactgaatta

SEQ ID NO: 233 - DNA, 107 bp, Artificial Sequence
  Synthetic oligonucleotide
gagccaatacctactgctccaagtcagatgtggatatgttttgccgagaggtgtccattctctgccagctcaatcatccctgcgtaattcagtttgtgggtgcttgc

SEQ ID NO: 234 - DNA, 24 bp, Artificial Sequence
  Synthetic oligonucleotide
tctgccattacctctaggatctgg

SEQ ID NO: 235 - DNA, 25 bp, Artificial Sequence
  Synthetic oligonucleotide
ggcagaagtaagcatacacctgaaa

SEQ ID NO: 236 - DNA, 108 bp, Artificial Sequence
  Synthetic oligonucleotide
tctgccattacctctaggatctggatcacccatagtatttgcatctggaggggagctcattactttaacagggaagaatgcaatttcaggtgtatgcttacttctgcc

SEQ ID NO: 237 - DNA, 22 bp, Artificial Sequence
  Synthetic oligonucleotide
cgttttgggatgtggattgagt

SEQ ID NO: 238 - DNA, 21 bp, Artificial Sequence
  Synthetic oligonucleotide
accgctttcatggagctaaca

SEQ ID NO: 239 - DNA, 128 bp, Artificial Sequence
  Synthetic oligonucleotide
cgttttgggatgtggattgagtatctcagaagataacctcttatcctggccattcaacctgatgtgttacatgtttatttgtttagaatcttccatcactaccaaaatgttagctccatgaaagcggt

SEQ ID NO: 240 - DNA, 2955 bp, Homo sapiens
gccgactctcctggatgcctggggttggagaagggtgcctccaaccccctgacccatgccccggaaagacagaaattcatccagagcagaatcagcccaatgccaggtgctttcatgtgttattcatggcatcttactaatggcccgtgagatagctgttgttgtcctccccctatcgcaggagagctacgtccaggcagcttcagatgcctccagagccatcgacatcaactcctcggacatcaaggctctgtatcggcgatgccaggcactggagcacctggggaagctggaccaggccttcaaagacgtgcagcgttgtgccaccctcgagccacggaaccagaacttccaggagatgctgaggagactcaacaccagcattcaggagaagctccgagtgcagttctccacagactcgagggtacagaagatgtttgagatcctcttggatgaaaacagtgaggctgataagcgggaaaaggctgccaacaatctcattgtcctaggccgtgaggaagcaggggctgagaagatcttccagaacaatggagtagccttgctactgcagcttctggacactaagaagcctgagctggtgctggctgcagtgcggaccctgtcgggcatgtgcagcggccaccaagccagagccacagtgattctgcatgcagtgcggatagaccgaatctgtagcctcatggccgtggagaatgaggagatgtctctggctgtctgcaacctgctccaagccatcattgactccttgtctggggaggacaagcgggagcatcgagggaaggaggaggccctggttctagacaccaagaaggacctgaagcagatcaccagccacctgctggacatgctagtcagcaagaaggtgtctggccagggcagggatcaggcgctgaacctgctcaataagaatgttcccaggaaggaccttgccattcatgacaactcacgtaccatctatgtggtggataatggtctgaggaagatcctgaaggttgtggggcaggttccagatctgccatcctgcctgcccctgactgacaacacccgcatgctggcctctatcctcatcaacaagctctatgatgacctgcgctgtgacccggagcgcgatcacttccgcaagatctgtgaggaatatatcacgggcaagtttgacccccaggacatggacaagaacttgaatgccatccagacagtgtcagggatcctgcagggcccctttgacctgggcaaccagctgctgggactgaaaggtgtgatggagatgatggtggcactatgtggctcagagcgcgagacggaccagctggtggccgtggaggccctcatccatgcctccacgaagctcagccgcgccaccttcatcatcaccaatggagtgtcactgctcaaacagatctacaagaccaccaaaaatgagaagatcaagatccgcacactggtgggactctgtaagctcggctctgcaggtggcacagactacggtctcaggcagtttgcggaagggtcgacagaaaaactggccaaacagtgtcgcaagtggctgtgcaatatgtccatagacactcggacccgacgctgggcagtggagggcctggcctacctcacgctggacgctgatgtgaaggacgactttgtccaggacgtccctgccctgcaggccatgtttgagctggccaaggcaggtaccagtgacaagaccatcctgtactcggtggccaccaccctggtgaactgcaccaacagctacgatgtcaaggaggtcatcccagagcttgtccagctcgccaagttctccaagcagcatgtgcccgaggaacaccccaaggacaagaaggactttatagacatgcgggtgaagcggcttctgaaggcgggtgtcatctctgccctggcttgcatggtgaaagcagatagtgccatcctcactgaccagaccaaggagctgctggccagggtattcctggcactgtgtgacaacccaaaggaccgaggcaccattgtggctcaaggtggtggcaaggccctgattcccctggctttggagggcacagatgtgggcaaggtgaaggcagcccacgctctagcaaagatcgctgctgtctccaatccggacattgcttttcctggggagcgggtgtatgaggtggtgcggccccttgtaagactcttggacacacagagggatgggcttcagaactatgaggctctcctaggcctcaccaacctgtctgggcggagtgacaaactccggcagaagatctttaaggagagggccttgccagacatcgagaactacatgtttgagaatcatgatcagctgcggcaggcggccaccgagtgcatgtgcaacatggtgctccacaaggaggtacaggaaaggttcttggctgacgggaatgaccggctgaagctggtggtgctgctctgcggggaggatgatgataaggtgcagaatgcggctgcaggggctctggccatgctgacagcagcacacaagaaactgtgcctcaagatgactcaagtgacaacccagtggttggagatcctccagcggctttgcctgcacgaccagctgtctgtccaacaccggggcctggtcattgcctacaacctactggcagccgatgctgagctggccaagaagctggtggagagtgagctgctggagatcctgactgtggtgggcaaacaggagccagatgagaagaaggcagaagtggttcagacagcccgagaatgtctcatcaagtgcatggattatggtttcattaaaccagtgtcttagacagcgaccctcagggatgctgggagtggtcctgtactgtgcagagtcctgggttggttgggttctcctggagagtcaggtcatctagggatcatagcagtgacaatgaagtctcaatataaaggaaagacttgattgttctctga

SEQ ID NO: 241 - DNA, 1956 bp, Homo sapiens
agactgaggctggctggctggaggttgacacaggagtgctcaggggagcagcatcacaagagggcagatcgaaagcatcgtccttgctgaaaaaatggcagaggtggaagcggtacagctgaaggaggaaggaaaccggcatttccagctccaggactacaaggccgccacaaatagctacagccaggccctgaagctgaccaaggacaaggccctgctggccacgctttatcggaaccgggcagcctgtggcctgaaaacggagagctacgtccaggcagcttcagatgcctccagagccatcgacatcaactcctcggacatcaaggctctgtatcggcgatgccaggcactggagcacctggggaagctggaccaggccttcaaagacgtgcagcgttgtgccaccctcgagccacggaaccagaacttccaggagatgctgaggagactcaacaccagcattcaggagaagctccgagtgcagttctccacagactcgagggtacagaagatgtttgagatcctcttggatgaaaacagtgaggctgataagcgggaaaaggctgccaacaatctcattgtcctaggccgtgaggaagcaggggctgagaagatcttccagaacaatggagtagccttgctactgcagcttctggacactaagaagcctgagctggtgctggctgcagtgcggaccctgtcgggcatgtgcagcggccaccaagccagagccacagtgattctgcatgcagtgcggatagaccgaatctgtagcctcatggccgtggagaatgaggagatgtctctggctgtctgcaacctgctccaagccatcattgactccttgtctggggaggacaagcgggagcatcgagggaaggaggaggccctggttctagacaccaagaaggacctgaagcagatcaccagccacctgctggacatgctagtcagcaagaaggtgtctggccagggcagggatcaggcgctgaacctgctcaataagaatgttcccaggaaggaccttgccattcatgacaactcacgtaccatctatgtggtggataatggtctgaggaagatcctgaaggttgtggggcaggttccagatctgccatcctgcctgcccctgactgacaacacccgcatgctggcctctatcctcatcaacaagctctatgatgacctgcgctgtgacccggagcgcgatcacttccgcaagatctgtgaggaatatatcacgggcaagtttgacccccaggacatggacaagaacttgaatgccatccagacagtgtcagggatcctgcagggcccctttgacctgggcaaccagctgctgggactgaaaggtgtgatggagatgatggtggcactatgtggctcagagcgcgagacggaccagctggtggccgtggaggccctcatccatgcctccacgaagctcagccgcgccaccttcatcatcaccaatggagtgtcactgctcaaacagatctacaagaccaccaaaaatgagaagatcaagatccgcacactggtgggactctgtaagctcggctctgcaggtggcacagactacggtctcaggcagtttgcggaagggtcgacagaaaaactggccaaacagtgtcgcaagtggctgtgcaatatgtccatagacactcggacccgacgctgggcagtggagggcctggcctacctcacgctggacgctgatgtgaaggacgactttgtccaggacgtccctgccctgcaggccatgtttgagctggccaaggcaggtgtcggggagtctggcccgaccacaaacctcaggaaaggtctgctgggtccagacccacagggaatggatcccagtcttcctcctggctctactccttacccctgtataaacatgattggttatttccccctttctggccctcattttacctgattgtaaaatggact

SEQ ID NO: 242 - DNA, 152 bp, Homo sapiens
cggtacagctgaaggaggaaggaaaccggcatttccagctccaggactacaaggccgccacaaatagctacagccaggccctgaagctgaccaaggacaaggccctgctggccacgctttatcggaaccgggcagcctgtggcctgaaaacg

SEQ ID NO: 243 - DNA, 181 bp, Homo sapiens
gccgactctcctggatgcctggggttggagaagggtgcctccaaccccctgacccatgccccggaaagacagaaattcatccagagcagaatcagcccaatgccaggtgctttcatgtgttattcatggcatcttactaatggcccgtgagatagctgttgttgtcctccccctatcgcag

SEQ ID NO: 244 - DNA, 176 bp, Homo sapiens
ccatcgacatcaactcctcggacatcaaggctctgtatcggcgatgccaggcactggagcacctggggaagctggaccaggccttcaaagacgtgcagcgttgtgccaccctcgagccacggaaccagaacttccaggagatgctgaggagactcaacaccagcattcaggagaag

SEQ ID NO: 245 - DNA, 168 bp, Homo sapiens
gctgccaacaatctcattgtcctaggccgtgaggaagcaggggctgagaagatcttccagaacaatggagtagccttgctactgcagcttctggacactaagaagcctgagctggtgctggctgcagtgcggaccctgtcgggcatgtgcagcggccaccaagccaga

SEQ ID NO: 246 - DNA, 169 bp, Homo sapiens
gccacagtgattctgcatgcagtgcggatagaccgaatctgtagcctcatggccgtggagaatgaggagatgtctctggctgtctgcaacctgctccaagccatcattgactccttgtctggggaggacaagcgggagcatcgagggaaggaggaggccctggttctag

SEQ ID NO: 247 - DNA, 171 bp, Homo sapiens
acaccaagaaggacctgaagcagatcaccagccacctgctggacatgctagtcagcaagaaggtgtctggccagggcagggatcaggcgctgaacctgctcaataagaatgttcccaggaaggaccttgccattcatgacaactcacgtaccatctatgtggtggataatg

SEQ ID NO: 248 - DNA, 172 bp, Homo sapiens
gtctgaggaagatcctgaaggttgtggggcaggttccagatctgccatcctgcctgcccctgactgacaacacccgcatgctggcctctatcctcatcaacaagctctatgatgacctgcgctgtgacccggagcgcgatcacttccgcaagatctgtgaggaatatatcac

SEQ ID NO: 249 - DNA, 301 bp, Homo sapiens
gggcaagtttgacccccaggacatggacaagaacttgaatgccatccagacagtgtcagggatcctgcagggcccctttgacctgggcaaccagctgctgggactgaaaggtgtgatggagatgatggtggcactatgtggctcagagcgcgagacggaccagctggtggccgtggaggccctcatccatgcctccacgaagctcagccgcgccaccttcatcatcaccaatggagtgtcactgctcaaacagatctacaagaccaccaaaaatgagaagatcaagatccgcacactggtg

SEQ ID NO: 250 - DNA, 142 bp, Homo sapiens
gtggctgtgcaatatgtccatagacactcggacccgacgctgggcagtggagggcctggcctacctcacgctggacgctgatgtgaaggacgactttgtccaggacgtccctgccctgcaggccatgtttgagctggccaag

SEQ ID NO: 251 - DNA, 167 bp, Homo sapiens
gtcggggagtctggcccgaccacaaacctcaggaaaggtctgctgggtccagacccacagggaatggatcccagtcttcctcctggctctactccttacccctgtataaacatgattggttatttccccctttctggccctcattttacctgattgtaaaatggact

SEQ ID NO: 252 - DNA, 141 bp, Homo sapiens
accagtgacaagaccatcctgtactcggtggccaccaccctggtgaactgcaccaacagctacgatgtcaaggaggtcatcccagagcttgtccagctcgccaagttctccaagcagcatgtgcccgaggaacaccccaag

SEQ ID NO: 253 - DNA, 128 bp, Homo sapiens
gacaagaaggactttatagacatgcgggtgaagcggcttctgaaggcgggtgtcatctctgccctggcttgcatggtgaaagcagatagtgccatcctcactgaccagaccaaggagctgctggccag

SEQ ID NO: 254 - DNA, 156 bp, Homo sapiens
gtacaggaaaggttcttggctgacgggaatgaccggctgaagctggtggtgctgctctgcggggaggatgatgataaggtgcagaatgcggctgcaggggctctggccatgctgacagcagcacacaagaaactgtgcctcaagatgactcaagtg

SEQ ID NO: 255 - DNA, 3019 bp, Homo sapiens
cggctttgcctgcacgaccagctgtctgtccaacaccggggcctggtcattgcctacaacctactggcagccgatgctgagctggccaagaagctggtggagagtgagctgctggagatcctgactgtggtgggcaaacaggagccagatgagaagaaggcagaagtggttcagacagcccgagaatgtctcatcaagtgcatggattatggtttcattaaaccagtgtcttagacagcgaccctcagggatgctgggagtggtcctgtactgtgcagagtcctgggttggttgggttctcctggagagtcaggtcatctagggatcatagcagtgacaatgaagtctcaatataaaggaaagacttgattgttctctgagttgtgagtcttctcctttgtcctgacagagtttggatgtttcactctctctcttgcttcctgtctccttatatttgtcatgtttcagaaattcccagaataattttcacccatgattagaaataggttggatcattctttctgtacccattctgaaggccaggataataggttgaagttctttatattttggaaaatggattttgtggtaaggtaggaactaacgggtgtgtgcatataaaggttaatgctgttgtatttggatgctgatctgtgctgttttcatgacctctctcccacactatttgaatcagtctgttcagaactgtgtgtttcacctgcagtgctctgtcccagtcccatgcccattcccatcacttcactgtgatggaaatagagtttatgttcaacagtggggcatgacctctgcaatttgcttgggaaagtcttccaagcaagatggacatgatgaatacaaataagatctactcaaagtacttcaaacaaaaaataaataattcattggctcatgtatcttggccacccagggaaggtctgacattgttagttagatccagagtttcaaatgtcatcaccatggatgtgtctttttctctctctcatttcccctccccatatcttgtcttttatttattataggtttgtctcattccctggcaggctctctccctgtgataggaaagagagtccccagcagccccagggtgacatagttgttatagttcattatggaatagaagagagaagagcattctcaataacccggcaaagttcccagggatgactctgatatgtctatgtctcaggtcacatttccatctatgaaccaatcatattcagaggtggaatgctaattggccaggcctgggtcatatatacaagtctagggaagaaatgagcttcatccctgtccaattgacatggactgattaggggtattaatggaagaggtgtgccaccacaaaagaatgtaccctgggcagatcaaagaacatattctgtatgtcaggcttggccacaaaagaatgacacaagtaatatgctgtagatcagaacctctctgctaatattgcctttttagcatggttaagatagctaagatctagtactgtcactccagtatgtcccaattctacctacgtttattgaagggtcaacagttctgatctcagcattgggtaaagggtgggacattcagatttacggtccttgataaaaacaatttacaacgttccgttgtgtaataaatgtaagtgtacatatgcctgggacatcagctggaaaagggacagactatcagagagttgcactgttgcggtatgggccaaatccaacataatacccgctgtacctctagagaactaaaaccttaatttctcagatcttttctgcactaatggtctttacatacagcctacattttaactaactcttgcatgggcttgtttcacagcaggaaactatattcatcatatccttattatgatagagaatgacaacattcaaaagggtgtggtgcttctgaaaatatacacaataaatggcatgatttgacctctgtcaaattcttctttcctgctttcctcatatgtgggcttgagtgggtgtatttagctcctttaaaggaaactctttcacaggcagggccacactcttactcgttttgatctcatgaggttacatctgccagtggcgttcagggccagttagataaagttcatcttgtttggattggagagactttttttttttttttttttgagacagggtcttgctctgtaacctaggctcaagtgcagtgcagtggtgcagtcatggctcaccgcagccttggtctcctgggctcaagtgatctttctacttcagcctcctcaatagctgggactacaggcacacaccaccacccccaggtaattaaaaaatttttttttgtagagacagggtctcactatgttgcccagtctgggagaccacattctttaacatgaatattttgtaaatagtccaagaagatctagaggaaagctgaattccttcatctgatttttgccactgggctgcctcccattgtctggctaagtcttcatgggaggcctttaagaaaagctgtgacaaatacctttgtttcccactgcctaggttacataagtagttggcttttccagcctagactcttatattgttgcttgtaagagggcccttctttgcagatttcttttttttttcatctatgctttcaaataggcagatgctagcctgagatcatctctttattgaggtagtttgcccaaaaacagcaaggaacagccaatacctgccaactttctaggtttttccaaccaattctcttacagaaaatctataaatatcttagaggtatgtttcttagtttcccaacatataatgggaaaattcatcatttatattattacttttaactgaagtatgttgttatcagagaacttggtctgtgtcatattaatcctttgccatttgagaagcatacagttttaaagtttatatctccaaaaggatattttgtaaaaatttaacccctttgactcttagtcttctcatttgtgaaaggtaaataataaagaatttctgttcctagtaag

SEQ ID NO: 256 - DNA, 94 bp, Homo sapiens
agactgaggctggctggctggaggttgacacaggagtgctcaggggagcagcatcacaagagggcagatcgaaagcatcgtccttgctgaaaaa

SEQ ID NO: 257 - DNA, 16 bp, Homo sapiens
atggcagaggtggaag

SEQ ID NO: 258 - DNA, 37 bp, Homo sapiens
gagagctacgtccaggcagcttcagatgcctccagag

SEQ ID NO: 259 - DNA, 90 bp, Homo sapiens
ctccgagtgcagttctccacagactcgagggtacagaagatgtttgagatcctcttggatgaaaacagtgaggctgataagcgggaaaag

SEQ ID NO: 260 - DNA, 95 bp, Homo sapiens
ggactctgtaagctcggctctgcaggtggcacagactacggtctcaggcagtttgcggaagggtcgacagaaaaactggccaaacagtgtcgcaa

SEQ ID NO: 261 - DNA, 6 bp, Homo sapiens
gcaggt

SEQ ID NO: 262 - DNA, 67 bp, Homo sapiens
ggtattcctggcactgtgtgacaacccaaaggaccgaggcaccattgtggctcaaggtggtggcaag

SEQ ID NO: 263 - DNA, 114 bp, Homo sapiens
gccctgattcccctggctttggagggcacagatgtgggcaaggtgaaggcagcccacgctctagcaaagatcgctgctgtctccaatccggacattgcttttcctggggagcgg

SEQ ID NO: 264 - DNA, 116 bp, Homo sapiens
gtgtatgaggtggtgcggccccttgtaagactcttggacacacagagggatgggcttcagaactatgaggctctcctaggcctcaccaacctgtctgggcggagtgacaaactccg

SEQ ID NO: 265 - DNA, 118 bp, Homo sapiens
gcagaagatctttaaggagagggccttgccagacatcgagaactacatgtttgagaatcatgatcagctgcggcaggcggccaccgagtgcatgtgcaacatggtgctccacaaggag

SEQ ID NO: 266 - DNA, 27 bp, Homo sapiens
acaacccagtggttggagatcctccag

SEQ ID NO: 267 - protein, 931 aa, Homo sapiens
MAEVEAVQLKEEGNRHFQLQDYKAATNSYSQALKLTKDKALLATLYRNRAACGLKTESYVQAASDASRAIDINSSDIKALYRRCQALEHLGKLDQAFKDVQRCATLEPRNQNFQEMLRRLNTSIQEKLRVQFSTDSRVQKMFEILLDENSEADKREKAANNLIVLGREEAGAEKIFQNNGVALLLQLLDTKKPELVLAAVRTLSGMCSGHQARATVILHAVRIDRICSLMAVENEEMSLAVCNLLQAIIDSLSGEDKREHRGKEEALVLDTKKDLKQITSHLLDMLVSKKVSGQGRDQALNLLNKNVPRKDLAIHDNSRTIYVVDNGLRKILKVVGQVPDLPSCLPLTDNTRMLASILINKLYDDLRCDPERDHFRKICEEYITGKFDPQDMDKNLNAIQTVSGILQGPFDLGNQLLGLKGVMEMMVALCGSERETDQLVAVEALIHASTKLSRATFIITNGVSLLKQIYKTTKNEKIKIRTLVGLCKLGSAGGTDYGLRQFAEGSTEKLAKQCRKWLCNMSIDTRTRRWAVEGLAYLTLDADVKDDFVQDVPALQAMFELAKAGTSDKTILYSVATTLVNCTNSYDVKEVIPELVQLAKFSKQHVPEEHPKDKKDFIDMRVKRLLKAGVISALACMVKADSAILTDQTKELLARVFLALCDNPKDRGTIVAQGGGKALIPLALEGTDVGKVKAAHALAKIAAVSNPDIAFPGERVYEVVRPLVRLLDTQRDGLQNYEALLGLTNLSGRSDKLRQKIFKERALPDIENYMFENHDQLRQAATECMCNMVLHKEVQERFLADGNDRLKLVVLLCGEDDDKVQNAAAGALAMLTAAHKKLCLKMTQVTTQWLEILQRLCLHDQLSVQHRGLVIAYNLLAADAELAKKLVESELLEILTVVGKQEPDEKKAEVVQTARECLIKCMDYGFIKPVS

SEQ ID NO: 268 - protein, 917 aa, Homo sapiens
MPRKDRNSSRAESAQCQVLSCVIHGILLMAREIAVVVLPLSQESYVQAASDASRAIDINSSDIKALYRRCQALEHLGKLDQAFKDVQRCATLEPRNQNFQEMLRRLNTSIQEKLRVQFSTDSRVQKMFEILLDENSEADKREKAANNLIVLGREEAGAEKIFQNNGVALLLQLLDTKKPELVLAAVRTLSGMCSGHQARATVILHAVRIDRICSLMAVENEEMSLAVCNLLQAIIDSLSGEDKREHRGKEEALVLDTKKDLKQITSHLLDMLVSKKVSGQGRDQALNLLNKNVPRKDLAIHDNSRTIYVVDNGLRKILKVVGQVPDLPSCLPLTDNTRMLASILINKLYDDLRCDPERDHFRKICEEYITGKFDPQDMDKNLNAIQTVSGILQGPFDLGNQLLGLKGVMEMMVALCGSERETDQLVAVEALIHASTKLSRATFIITNGVSLLKQIYKTTKNEKIKIRTLVGLCKLGSAGGTDYGLRQFAEGSTEKLAKQCRKWLCNMSIDTRTRRWAVEGLAYLTLDADVKDDFVQDVPALQAMFELAKAGTSDKTILYSVATTLVNCTNSYDVKEVIPELVQLAKFSKQHVPEEHPKDKKDFIDMRVKRLLKAGVISALACMVKADSAILTDQTKELLARVFLALCDNPKDRGTIVAQGGGKALIPLALEGTDVGKVKAAHALAKIAAVSNPDIAFPGERVYEVVRPLVRLLDTQRDGLQNYEALLGLTNLSGRSDKLRQKIFKERALPDIENYMFENHDQLRQAATECMCNMVLHKEVQERFLADGNDRLKLVVLLCGEDDDKVQNAAAGALAMLTAAHKKLCLKMTQVTTQWLEILQRLCLHDQLSVQHRGLVIAYNLLAADAELAKKLVESELLEILTVVGKQEPDEKKAEVVQTARECLIKCMDYGFIKPVS

SEQ ID NO: 269 - protein, 615 aa, Homo sapiens
MAEVEAVQLKEEGNRHFQLQDYKAATNSYSQALKLTKDKALLATLYRNRAACGLKTESYVQAASDASRAIDINSSDIKALYRRCQALEHLGKLDQAFKDVQRCATLEPRNQNFQEMLRRLNTSIQEKLRVQFSTDSRVQKMFEILLDENSEADKREKAANNLIVLGREEAGAEKIFQNNGVALLLQLLDTKKPELVLAAVRTLSGMCSGHQARATVILHAVRIDRICSLMAVENEEMSLAVCNLLQAIIDSLSGEDKREHRGKEEALVLDTKKDLKQITSHLLDMLVSKKVSGQGRDQALNLLNKNVPRKDLAIHDNSRTIYVVDNGLRKILKVVGQVPDLPSCLPLTDNTRMLASILINKLYDDLRCDPERDHFRKICEEYITGKFDPQDMDKNLNAIQTVSGILQGPFDLGNQLLGLKGVMEMMVALCGSERETDQLVAVEALIHASTKLSRATFIITNGVSLLKQIYKTTKNEKIKIRTLVGLCKLGSAGGTDYGLRQFAEGSTEKLAKQCRKWLCNMSIDTRTRRWAVEGLAYLTLDADVKDDFVQDVPALQAMFELAKAGVGESGPTTNLRKGLLGPDPQGMDPSLPPGSTPYPCINMIGYFPLSGPHFT

SEQ ID NO: 270 - DNA, 19 bp, Artificial Sequence
  Synthetic oligonucleotide
gtctggcccgaccacaaac

SEQ ID NO: 271 - DNA, 23 bp, Artificial Sequence
  Synthetic oligonucleotide
ggtaaggagtagagccaggagga

SEQ ID NO: 272 - DNA, 92 bp, Artificial Sequence
  Synthetic oligonucleotide
gtctggcccgaccacaaacctcaggaaaggtctgctgggtccagacccacagggaatggatcccagtcttcctcctggctctactccttacc

SEQ ID NO: 273 - DNA, 20 bp, Artificial Sequence
  Synthetic oligonucleotide
accatcctgtactcggtggc

SEQ ID NO: 274 - DNA, 21 bp, Artificial Sequence
  Synthetic oligonucleotide
catgctgcttggagaacttgg

SEQ ID NO: 275 - DNA, 109 bp, Artificial Sequence
  Synthetic oligonucleotide
accatcctgtactcggtggccaccaccctggtgaactgcaccaacagctacgatgtcaaggaggtcatcccagagcttgtccagctcgccaagttctccaagcagcatg

SEQ ID NO: 276 - DNA, 1029 bp, Homo sapiens
aaagggtattgggtctgacagataagagctcctgcactctaccagccagctactgacagacataggtctggctccagtggaggggcagcagccagtgagcccagcctggggtggcccactcctgctgcctccaggatgtcccctgtttccccagcccctctgctgtgccctcggccccagaagctggcgagactgcttctctggaacagcatcacgcaggcctgcccatcggcaccgctgtgcaccaggccttctggggatacagatgtcaaccaggtgataaggctgagacataaaggaggctgggccctgccaccacgacagcagccacacctctgcagagagaatggccagcaggaaggcggggacccggggcaaggtggcagccaccaagcaggcccaacgtggttcttccaacgtcttttccatgtttgaacaagcccagatacaggagttcaaagaagccttcagctgtatcgaccagaatcgtgatggcatcatctgcaaggcagacctgagggagacctactcccagctggggaaggtgagtgtcccagaggaggagctggacgccatgctgcaagagggcaagggccccatcaacttcaccgtcttcctcacgctctttggggagaagctcaatgggacagaccccgaggaagccatcctgagtgccttccgcatgtttgaccccagcggcaaaggggtggtgaacaaggatgagttcaagcagcttctcctgacccaggcagacaagttctctccagctgaggtgaggctgcccagccccttcaatactcatccccagcaccttctctgggccttcacccatgacccagagcccagtaccagtgaggcagttgctggaagggtggagcagatgttcgccctgacacccatggacctggcggggaacatcgactacaagtcactgtgctacatcatcacccatggagacgagaaagaggaatgaggggcagggccaggcccacgggggggcacctcaataaactctgttgcaaaattgga

SEQ ID NO: 277 - DNA, 1025 bp, Homo sapiens
aaagggtattgggtctgacagataagagctcctgcactctaccagccagctactgacagacataggtctggctccagtggaggggcagcagccagtgagcccagcctggggtggcccactcctgctgcctccaggatgtcccctgtttccccagcccctctgctgtgccctcggccccagaagctggcgagactgcttctctggaacagcatcacgcaggcctgcccatcggcaccgctgtgcaccaggccttctggggatacagatgtcaaccaggtgataaggctgagacataaaggaggctgggccctgccaccacgacagcagccacacctctgcagagagaatggccagcaggaaggcggggacccggggcaaggtggcagccaccaagcaggcccaacgtggttcttccaacgtcttttccatgtttgaacaagcccagatacaggagttcaaagaagccttcagctgtatcgaccagaatcgtgatggcatcatctgcaaggcagacctgagggagacctactcccagctggggaaggtgagtgtcccagaggaggagctggacgccatgctgcaagagggcaagggccccatcaacttcaccgtcttcctcacgctctttggggagaagctcaatgggacagaccccgaggaagccatcctgagtgccttccgcatgtttgaccccagcggcaaaggggtggtgaacaaggatgaccaacccttccctgcgccatgggagcctccgtacccaccttccctgtgcagtcactcccccgcagtctcctgctcagaccctcctcaccccccaggttcaagcagcttctcctgacccaggcagacaagttctctccagctgaggtggagcagatgttcgccctgacacccatggacctggcggggaacatcgactacaagtcactgtgctacatcatcacccatggagacgagaaagaggaatgaggggcagggccaggcccacgggggggcacctcaataaactctgttgcaaaattgga

SEQ ID NO: 278 - DNA, 999 bp, Homo sapiens
aaagggtattgggtctgacagataagagctcctgcactctaccagccagctactgacagacataggtctggctccagtggaggggcagcagccagtgagcccagcctggggtggcccactcctgctgcctccaggatgtcccctgtttccccagcccctctgctgtgccctcggccccagaagctggcgagactgcttctctggaacagcatcacgcaggcctgcccatcggcaccgctgtgcaccaggccttctggggatacagatgtcaaccaggtgataaggctgagacataaaggaggctgggccctgccaccacgacagcagccacacctctgcagagagaatggccagcaggaaggcggggacccggggcaaggtggcagccaccaagcaggcccaacgtggttcttccaacgtcttttccatgtttgaacaagcccagatacaggagttcaaagaagtctctcccccacctcccaccttccctagagctgggggctgctcccacctgaaggcccccatcccacaggccttcagctgtatcgaccagaatcgtgatggcatcatctgcaaggcagacctgagggagacctactcccagctggggaaggtgagtgtcccagaggaggagctggacgccatgctgcaagagggcaagggccccatcaacttcaccgtcttcctcacgctctttggggagaagctcaatgggacagaccccgaggaagccatcctgagtgccttccgcatgtttgaccccagcggcaaaggggtggtgaacaaggatgagttcaagcagcttctcctgacccaggcagacaagttctctccagctgaggtggagcagatgttcgccctgacacccatggacctggcggggaacatcgactacaagtcactgtgctacatcatcacccatggagacgagaaagaggaatgaggggcagggccaggcccacgggggggcacctcaataaactctgttgcaaaattgga

SEQ ID NO: 279 - DNA, 881 bp, Homo sapiens
aaagggtattgggtctgacagataagagctcctgcactctaccagccagctactgacagacataggtctggctccagtggaggggcagcagccagtgagcccagcctggggtggcccactcctgctgcctccaggatgtcccctgtttccccagcccctctgctgtgccctcggccccagaagctggcgagactgcttctctggaacagcatcacgcaggcctgcccatcggcaccgctgtgcaccaggccttctggggatacagatgtcaaccaggtgataaggctgagacataaaggaggctgggccctgccaccacgacagcagccacacctctgcagagagaatggccagcaggaaggcggggacccggggcaaggtggcagccaccaagcaggcccaacgtggttcttccaacgtcttttccatgtttgaacaagcccagatacaggagttcaaagaagccttcagctgtatcgaccagaatcgtgatggcatcatctgcaaggcagacctgagggagacctactcccagctggggaaggtgagtgtcccagaggaggagctggacgccatgctgcaagagggcaagggccccatcaacttcaccgtcttcctcacgctctttggggagaagctcaatgggacagaccccgaggaagccatcctgagtgccttccgcatgtttgaccccagcggcaaaggggtggtgaacaaggatgagtggagcagatgttcgccctgacacccatggacctggcggggaacatcgactacaagtcactgtgctacatcatcacccatggagacgagaaagaggaatgaggggcagggccaggcccacgggggggcacctcaataaactctgttgcaaaattgga

SEQ ID NO: 280 - DNA, 1021 bp, Homo sapiens
aaagggtattgggtctgacagataagagctcctgcactctaccagccagctactgacagacataggtctggctccagtggaggggcagcagccagtgagcccagcctggggtggcccactcctgctgcctccaggatgtcccctgtttccccagcccctctgctgtgccctcggccccagaagctggcgagactgcttctctggaacagcatcacgcaggcctgcccatcggcaccgctgtgcaccaggccttctggggatacagatgtcaaccaggtgataaggctgagacataaaggaggctgggccctgccaccacgacagcagccacacctctgcagagagaatggccagcaggaaggcggggacccggggcaaggtggcagccaccaagcaggcccaacgtggttcttccaacgtcttttccatgtttgaacaagcccagatacaggagttcaaagaagccttcagctgtatcgaccagaatcgtgatggcatcatctgcaaggcagacctgagggagacctactcccagctggcccctgtggccaggatggagggagggcggcctgggcccttctgggggacacccagggtccctgtgtgcacctcatgccccacccccaccagggaaggtgagtgtcccagaggaggagctggacgccatgctgcaagagggcaagggccccatcaacttcaccgtcttcctcacgctctttggggagaagctcaatgggacagaccccgaggaagccatcctgagtgccttccgcatgtttgaccccagcggcaaaggggtggtgaacaaggatgagttcaagcagcttctcctgacccaggcagacaagttctctccagctgaggtggagcagatgttcgccctgacacccatggacctggcggggaacatcgactacaagtcactgtgctacatcatcacccatggagacgagaaagaggaatgaggggcagggccaggcccacgggggggcacctcaataaactctgttgcaaaattgga

SEQ ID NO: 281 - DNA, 1284 bp, Homo sapiens
aaagggtattgggtctgacagataagagctcctgcactctaccagccagctactgacagacataggtctggctccagtggaggggcagcagccagtgagcccagcctggggtggcccactcctgctgcctccaggatgtcccctgtttccccagcccctctgctgtgccctcggccccagaagctggcgagactgcttctctggaacagcatcacgcaggcctgcccatcggcaccgctgtgcaccaggccttctggggatacagatgtcaaccaggtgataaggctgagacataaaggaggctgggccctgccaccacgacagcagccacacctctgcagagagaatggccagcaggaaggcggggacccggggcaaggtggcagccaccaagcaggcccaacgtggttcttccaacgtcttttccatgtttgaacaagcccagatacaggagttcaaagaagtctctcccccacctcccaccttccctagagctgggggctgctcccacctgaaggcccccatcccacaggccttcagctgtatcgaccagaatcgtgatggcatcatctgcaaggcagacctgagggagacctactcccagctgggtgcgtgcacccacctcccaccctgcgcactggggtccctactctgagctgctgggcgggtgggagtggctggggggacaggactctgctcccctgcttcccctcctccccgtctcctcacactgcccttccccccttgtcacgccttgcttccacttcaccttcccgacccacagctgcctctgcccctccagcccctgtggccaggatggagggagggcggcctgggcccttctgggggacacccagggtccctgtgtgcacctcatgccccacccccaccagggaaggtgagtgtcccagaggaggagctggacgccatgctgcaagagggcaagggccccatcaacttcaccgtcttcctcacgctctttggggagaagctcaatgggacagaccccgaggaagccatcctgagtgccttccgcatgtttgaccccagcggcaaaggggtggtgaacaaggatgagttcaagcagcttctcctgacccaggcagacaagttctctccagctgaggtggagcagatgttcgccctgacacccatggacctggcggggaacatcgactacaagtcactgtgctacatcatcacccatggagacgagaaagaggaatgaggggcagggccaggcccacgggggggcacctcaataaactctgttgcaaaattgga

SEQ ID NO: 282 - DNA, 276 bp, Homo sapiens
aaagggtattgggtctgacagataagagctcctgcactctaccagccagctactgacagacataggtctggctccagtggaggggcagcagccagtgagcccagcctggggtggcccactcctgctgcctccaggatgtcccctgtttccccagcccctctgctgtgccctcggccccagaagctggcgagactgcttctctggaacagcatcacgcaggcctgcccatcggcaccgctgtgcaccaggccttctggggatacagatgtcaaccag

SEQ ID NO: 283 - DNA, 194 bp, Homo sapiens
gtgcgtgcacccacctcccaccctgcgcactggggtccctactctgagctgctgggcgggtgggagtggctggggggacaggactctgctcccctgcttcccctcctccccgtctcctcacactgcccttccccccttgtcacgccttgcttccacttcaccttcccgacccacagctgcctctgcccctccag

SEQ ID NO: 284 - DNA, 1257 bp, Homo sapiens
gtggagcagatgttcgccctgacacccatggacctggcggggaacatcgactacaagtcactgtgctacatcatcacccatggagacgagaaagaggaatgaggggcagggccaggcccacgggggggcacctcaataaactctgttgcaaaattggaattgctgtggtgtcttgtctgtgacagatgggttggggaccagccaagggggatcccagggtctcagtgcgcacatcaccatgatcatggccaccatctacctcctgggagctggcccctcgccagctcaccttgattcactcccatgatgccaagtgaagtgtgaactatgatcatgcctagtttacagatgaggacactgaggcccagaaagtgtgagcatcttaccaaggccagccctctagaagaggagatggtgggatttacaccacctccaccaagcccaggaatgagccacaaagtgggcactgcccagctacttggggctgtgcagagaagaggctgcttgctgggcactcagcaaactctgcccaacagcccagcgggtgggcagcagccctgggacccccacacccaaccacacagcctcccctggcccactgctcgcaccccatctcaatacactggcttgggtgcctccctgcatgggccctttgtgaaaggcagagaggtacccatttgaaacacaaccagcttctcattgcaaatacaggcaaggcactaagacatgaggaacatggacaccaaagcaggggccaggtaacatgcaaatttctagaggaaatgcccagaacctggcatcatgcctcctgagcccctcatgcgccgtgaggggtaagagggtcagacagctggagtgtagggagacgacttctcaggagagaatagttagtgctcccgtcacccttcatctgagaacccaagagctagaggagaaagtgatcctcatgagtaccagaggagcagcaggggacatccaaagcaccagagagagaaacagagacagagagacaggcagtgacagctcaaacctcagccagatccagagcatacaaagtctcctgcctacaggacagcccagtaagagctctcagcttgcctccttccctccccacaagccctgctgcaatccctgtacctgggggtcagtgggaaggaggtgagcgagaaaggaggggcaccccttcctgaaggccccaagaggaaaggcgttttcacccagacaggtgttcagttttgattttatctggcgcctggc

SEQ ID NO: 285 - DNA, 73 bp, Homo sapiens
gtgataaggctgagacataaaggaggctgggccctgccaccacgacagcagccacacctctgcagagagaatg

SEQ ID NO: 286 - DNA, 96 bp, Homo sapiens
gccagcaggaaggcggggacccggggcaaggtggcagccaccaagcaggcccaacgtggttcttccaacgtcttttccatgtttgaacaagcccag

SEQ ID NO: 287 - DNA, 18 bp, Homo sapiens
atacaggagttcaaagaa

SEQ ID NO: 288 - DNA, 29 bp, Homo sapiens
gtctctcccccacctcccaccttccctag

SEQ ID NO: 289 - DNA, 40 bp, Homo sapiens
agctgggggctgctcccacctgaaggcccccatcccacag

SEQ ID NO: 290 - DNA, 76 bp, Homo sapiens
gccttcagctgtatcgaccagaatcgtgatggcatcatctgcaaggcagacctgagggagacctactcccagctgg

SEQ ID NO: 291 - DNA, 24 bp, Homo sapiens
cccctgtggccaggatggagggag

SEQ ID NO: 292 - DNA, 37 bp, Homo sapiens
ggcggcctgggcccttctgggggacacccagggtccc

SEQ ID NO: 293 - DNA, 30 bp, Homo sapiens
tgtgtgcacctcatgccccacccccaccag

SEQ ID NO: 294 - DNA, 85 bp, Homo sapiens
ggaaggtgagtgtcccagaggaggagctggacgccatgctgcaagagggcaagggccccatcaacttcaccgtcttcctcacgct

SEQ ID NO: 295 - DNA, 20 bp, Homo sapiens
ctttggggagaagctcaatg

SEQ ID NO: 296 - DNA, 6 bp, Homo sapiens
ggacag

SEQ ID NO: 297 - DNA, 73 bp, Homo sapiens
accccgaggaagccatcctgagtgccttccgcatgtttgaccccagcggcaaaggggtggtgaacaaggatga

SEQ ID NO: 298 - DNA, 95 bp, Homo sapiens
ccaacccttccctgcgccatgggagcctccgtacccaccttccctgtgcagtcactcccccgcagtctcctgctcagaccctcctcaccccccag

SEQ ID NO: 299 - DNA, 16 bp, Homo sapiens
gttcaagcagcttctc

SEQ ID NO: 300 - DNA, 33 bp, Homo sapiens
ctgacccaggcagacaagttctctccagctgag

SEQ ID NO: 301 - DNA, 99 bp, Homo sapiens
gtgaggctgcccagccccttcaatactcatccccagcaccttctctgggccttcacccatgacccagagcccagtaccagtgaggcagttgctggaagg

SEQ ID NO: 302 - protein, 175 aa, Homo sapiens
MASRKAGTRGKVAATKQAQRGSSNVFSMFEQAQIQEFKEAFSCIDQNRDGIICKADLRETYSQLGKVSVPEEELDAMLQEGKGPINFTVFLTLFGEKLNGTDPEEAILSAFRMFDPSGKGVVNKDEFKQLLLTQADKFSPAEVEQMFALTPMDLAGNIDYKSLCYIITHGDEKEE

SEQ ID NO: 303 - protein, 208 aa, Homo sapiens
MASRKAGTRGKVAATKQAQRGSSNVFSMFEQAQIQEFKEAFSCIDQNRDGIICKADLRETYSQLGKVSVPEEELDAMLQEGKGPINFTVFLTLFGEKLNGTDPEEAILSAFRMFDPSGKGVVNKDEFKQLLLTQADKFSPAEVRLPSPFNTHPQHLLWAFTHDPEPSTSEAVAGRVEQMFALTPMDLAGNIDYKSLCYIITHGDEKEE

SEQ ID NO: 304 - protein, 163 aa, Homo sapiens
MASRKAGTRGKVAATKQAQRGSSNVFSMFEQAQIQEFKEAFSCIDQNRDGIICKADLRETYSQLGKVSVPEEELDAMLQEGKGPINFTVFLTLFGEKLNGTDPEEAILSAFRMFDPSGKGVVNKDDQPFPAPWEPPYPPSLCSHSPAVSCSDPPHPPGSSSFS

SEQ ID NO: 305 - protein, 198 aa, Homo sapiens
MASRKAGTRGKVAATKQAQRGSSNVFSMFEQAQIQEFKEVSPPPPTFPRAGGCSHLKAPIPQAFSCIDQNRDGIICKADLRETYSQLGKVSVPEEELDAMLQEGKGPINFTVFLTLFGEKLNGTDPEEAILSAFRMFDPSGKGVVNKDEFKQLLLTQADKFSPAEVEQMFALTPMDLAGNIDYKSLCYIITHGDEKEE

SEQ ID NO: 306 - protein, 132 aa, Homo sapiens
MASRKAGTRGKVAATKQAQRGSSNVFSMFEQAQIQEFKEAFSCIDQNRDGIICKADLRETYSQLGKVSVPEEELDAMLQEGKGPINFTVFLTLFGEKLNGTDPEEAILSAFRMFDPSGKGVVNKDEWSRCSP

SEQ ID NO: 307 - protein, 144 aa, Homo sapiens
MASRKAGTRGKVAATKQAQRGSSNVFSMFEQAQIQEFKEAFSCIDQNRDGIICKADLRETYSQLAPVARMEGGRPGPFWGTPRVPVCTSCPTPTREGECPRGGAGRHAARGQGPHQLHRLPHALWGEAQWDRPRGSHPECLPHV

SEQ ID NO: 308 - protein, 102 aa, Homo sapiens
MASRKAGTRGKVAATKQAQRGSSNVFSMFEQAQIQEFKEVSPPPPTFPRAGGCSHLKAPIPQAFSCIDQNRDGIICKADLRETYSQLGACTHLPPCALGSLL

SEQ ID NO: 309 - DNA, 21 bp, Artificial Sequence
  Synthetic oligonucleotide
ggaaggtgagtgtcccagagg

SEQ ID NO: 310 - DNA, 23 bp, Artificial Sequence
  Synthetic oligonucleotide
gtgaggaagacggtgaagttgat

SEQ ID NO: 311 - DNA, 82 bp, Artificial Sequence
  Synthetic oligonucleotide
ggaaggtgagtgtcccagaggaggagctggacgccatgctgcaagagggcaagggccccatcaacttcaccgtcttcctcac

SEQ ID NO: 312 - DNA, 20 bp, Artificial Sequence
  Synthetic oligonucleotide
cccaccttccctagagctgg

SEQ ID NO: 313 - DNA, 21 bp, Artificial Sequence
  Synthetic oligonucleotide
aggtctgccttgcagatgatg

SEQ ID NO: 314 - DNA, 107 bp, Artificial Sequence
  Synthetic oligonucleotide
cccaccttccctagagctgggggctgctcccacctgaaggcccccatcccacaggccttcagctgtatcgaccagaatcgtgatggcatcatctgcaaggcagacct

SEQ ID NO: 315 - DNA, 23 bp, Artificial Sequence
  Synthetic oligonucleotide
aggcagacaagttctctccagct

SEQ ID NO: 316 - DNA, 19 bp, Artificial Sequence
  Synthetic oligonucleotide
ggtgaaggcccagagaagg

SEQ ID NO: 317 - DNA, 83 bp, Artificial Sequence
  Synthetic oligonucleotide
aggcagacaagttctctccagctgaggtgaggctgcccagccccttcaatactcatccccagcaccttctctgggccttcacc

SEQ ID NO: 318 - DNA, 17 bp, Artificial Sequence
  Synthetic oligonucleotide
tgtcccagcctccccac

SEQ ID NO: 319 - DNA, 19 bp, Artificial Sequence
  Synthetic oligonucleotide
gagtcacattcagggcccc

SEQ ID NO: 320 - DNA, 91 bp, Artificial Sequence
  Synthetic oligonucleotide
tgtcccagcctccccaccttctcagaccagcatgtggcccttaagtccacttgtaacactatacccatggttggggccctgaatgtgactc

SEQ ID NO: 321 - DNA, 21 bp, Artificial Sequence
  Synthetic oligonucleotide
caactctcctcaggttcccct

SEQ ID NO: 322 - DNA, 24 bp, Artificial Sequence
  Synthetic oligonucleotide
gagaaggaggaatgaagaaggctt

SEQ ID NO: 323 - DNA, 91 bp, Artificial Sequence
  Synthetic oligonucleotide
caactctcctcaggttcccctgaagtaattcattcttcctctacacctgaagctctagttgcctggaaagccttcttcattcctccttctc

SEQ ID NO: 324 - DNA, 50 bp, Homo sapiens
agtgggcactcggctccggacactgtaactcttgctctctaccttgctca

SEQ ID NO: 325 - DNA, 50 bp, Homo sapiens
atgccctttatcttgggcctcttgtctggaggtgtgaccaccactccatg

SEQ ID NO: 326 - protein, 46 aa, Artificial Sequence; identical to SEQ ID NO: 77
  Synthetic peptide

SEQ ID NO: 327 - protein, 44 aa, Artificial Sequence; identical to SEQ ID NO: 76
  Synthetic peptide

SEQ ID NO: 328 - protein, 44 aa, Artificial Sequence; identical to SEQ ID NO: 63
  Synthetic peptide

SEQ ID NO: 329 - protein, 10 aa, Artificial Sequence
  Synthetic peptide
AALAEGETPR

SEQ ID NO: 330 - protein, 198 aa, Artificial Sequence
  Synthetic peptide
GEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRAALAEGETPR

SEQ ID NO: 331 - protein, 60 aa, Artificial Sequence
  Synthetic peptide
QKGPLSCPSLPTFSDQHVALKSTCNTIPMVGALNVTHSWLFISPVTLHKEFFLSPVINYL

SEQ ID NO: 332 - protein, 30 aa, Artificial Sequence
  Synthetic peptide
SPPFPIWGDAKWSAWAPKQESSMHVASNSR

SEQ ID NO: 333 - protein, 202 aa, Artificial Sequence
  Synthetic peptide
GEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVSPPFPIWGDAKWSAWAPKQESSMHVASNSR

SEQ ID NO: 334 - protein, 15 aa, Artificial Sequence
  Synthetic peptide
RRAAPCTGYQSSVCV

SEQ ID NO: 335 - protein, 91 aa, Artificial Sequence
  Synthetic peptide
GEKRDLEIEVVLFHPNYNINGKKEAGIPEFYDYDVALIKLKNKLKYGQTIRPICLPCTEGTTRALRLPPTTTCQQQRRAAPCTGYQSSVCV

SEQ ID NO: 336 - protein, 15 aa, Artificial Sequence
  Synthetic peptide
GRAAPCTGYQSSVCV

SEQ ID NO: 337 - protein, 66 aa, Artificial Sequence
  Synthetic peptide
GEKRDLEIEVVLFHPNYNINGKKEAGIPEFYDYDVALIKLKNKLKYGQTIRGRAAPCTGYQSSVCV

SEQ ID NO: 338 - protein, 10 aa, Artificial Sequence
  Synthetic peptide
VRNGHPKEAL

SEQ ID NO: 339 - protein, 120 aa, Artificial Sequence
  Synthetic peptide
GEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKVRNGHPKEAL

SEQ ID NO: 340 - protein, 146 aa, Artificial Sequence
  Synthetic peptide
EEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 341 - protein, 12 aa, Artificial Sequence
  Synthetic peptide
GREIQGNKEHNS

SEQ ID NO: 342 - protein, 11 aa, Artificial Sequence
  Synthetic peptide
AVRRGLREGGA

SEQ ID NO: 343 - protein, 125 aa, Artificial Sequence
  Synthetic peptide
AVRRGLREGGAMAAARDPPEVSLREATQRKLRRFSELRGKLVARGEFWDIVAITAADEKQELAYNQQLSEKLKRKELPLGVQYHVFVDPAGAKIGNGGSTLCALQCLEKLYGDKWNSFTILLIHS

SEQ ID NO: 344 - protein, 7 aa, Artificial Sequence
  Synthetic peptide
AVRRGLR

SEQ ID NO: 345 - protein, 266 aa, Artificial Sequence
  Synthetic peptide
SAITSRIWITHSICIWRGAHYFNREECNFRCMLTSAILKESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYGYVSDPMSSMHFHSCRNSSSFEDSS

SEQ ID NO: 346 - protein, 13 aa, Artificial Sequence
  Synthetic peptide
MGNYKSRPTQTCT

SEQ ID NO: 347 - protein, 266 aa, Artificial Sequence; identical to SEQ ID NO: 345
  Synthetic peptide

SEQ ID NO: 348 - protein, 13 aa, Artificial Sequence
  Synthetic peptide
MGNYKSRPTQTCT

SEQ ID NO: 349 - protein, 4 aa, Artificial Sequence
  Synthetic peptide
DVTS

SEQ ID NO: 350 - protein, 224 aa, Artificial Sequence
  Synthetic peptide
SHNILLYEDGHAVVADFGESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYDVTS

SEQ ID NO: 351 - protein, 15 aa, Artificial Sequence
  Synthetic peptide
PSPCGLHFLIPWLTQ

SEQ ID NO: 352 - protein, 245 aa, Artificial sequence
  Synthetic peptide
SHNILLYEDGHAVVADFGESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEGRPEFSEVVMKLEECLCNIELMSPASSNSSGSLSPSSSSDCLVNRGGPGRSHVAALRSRFELEYALNARSYAALSQSAGQYSSQGLSLEEMKRSLQYTPIDKYGYVSDPMSSMHFHSCRNSSSFEDSS

SEQ ID NO: 353 - protein, 15 aa, Artificial Sequence
  Synthetic peptide
PSPCGLHFLIPWLTQ

SEQ ID NO: 354 - protein, 266 aa, Artificial Sequence; identical to SEQ ID NO: 345
  Synthetic peptide

SEQ ID NO: 355 - protein, 11 aa, Artificial Sequence
  Synthetic peptide
AVRRGLREGGA

SEQ ID NO: 356 - protein, 35 aa, Artificial Sequence
  Synthetic peptide
AKSRPSHYPVSSVYTETLKKKNEDRFGMWIEYLRR

SEQ ID NO: 357 - protein, 7 aa, Artificial Sequence
  Synthetic peptide
AVRRGLR

SEQ ID NO: 358 - protein, 152 aa, Artificial Sequence
  Synthetic peptide
SHNILLYEDGHAVVADFGESRFLQSLDEDNMTKQPGNLRWMAPEVFTQCTRYTIKADVFSYALCLWEILTGEIPFAHLKPAAAAADMAYHHIRPPIGYSIPKPISSLLIRGWNACPEAKSRPSHYPVSSVYTETLKKKNEDRFGMWIEYLRR

SEQ ID NO: 359 - protein, 125 aa, Artificial Sequence; identical to SEQ ID NO: 343
  Synthetic peptide

SEQ ID NO: 360 - protein, 13 aa, Artificial Sequence
  Synthetic peptide
MGNYKSRPTQTCT

SEQ ID NO: 361 - protein, 35 aa, Artificial Sequence; identical to SEQ ID NO: 356
  Synthetic peptide

SEQ ID NO: 362 - protein, 152 aa, Artificial Sequence; identical to SEQ ID NO: 358
  Synthetic peptide

SEQ ID NO: 363 - protein, 11 aa, Artificial Sequence
  Synthetic peptide
AVRRGLREGGA

SEQ ID NO: 364 - protein, 6 aa, Artificial Sequence
  Synthetic peptide
RYFFPK

SEQ ID NO: 365 - protein, 7 aa, Artificial Sequence
  Synthetic peptide
AVRRGLR

SEQ ID NO: 366 - protein, 125 aa, Artificial Sequence; identical to SEQ ID NO: 343
  Synthetic peptide

SEQ ID NO: 367 - protein, 13 aa, Artificial Sequence
  Synthetic peptide
MGNYKSRPTQTCT

SEQ ID NO: 368 - protein, 13 aa, Artificial Sequence
  Synthetic peptide
RCCTGWLSCYHPD

SEQ ID NO: 369 - protein, 12 aa, Artificial Sequence
  Synthetic peptide
CCTGWLSCYHPD

SEQ ID NO: 370 - protein, 3 aa, Artificial Sequence
  Synthetic peptide
RAS

SEQ ID NO: 371 - protein, 11 aa, Artificial Sequence
  Synthetic peptide
AVRRGLREGGA

SEQ ID NO: 372 - protein, 34 aa, Artificial Sequence
  Synthetic peptide
YGSFVLIYPWTFRRNYSCNTSEGFPLDEPSPFEI

SEQ ID NO: 373 - protein, 7 aa, Artificial Sequence
  Synthetic peptide
AVRRGLR

SEQ ID NO: 374 - protein, 125 aa, Artificial Sequence; identical to SEQ ID NO: 343
  Synthetic peptide

SEQ ID NO: 375 - protein, 13 aa, Artificial Sequence
  Synthetic peptide
MGNYKSRPTQTCT

SEQ ID NO: 376 - protein, 34 aa, Artificial Sequence; identical to SEQ ID NO: 372
  Synthetic peptide

SEQ ID NO: 377 - protein, 13 aa, Artificial Sequence
  Synthetic peptide
MGNYKSRPTQTCT

SEQ ID NO: 378 - protein, 3 aa, Artificial Sequence
  Synthetic peptide
NLK

SEQ ID NO: 379 - protein, 114 aa, Artificial Sequence
  Synthetic peptide
MAAARDPPEVSLREATQRKLRRFSELRGKLVARGEFWDIVAITAADEKQELAYNQQLSEKLKRKELPLGVQYHVFVDPAGAKIGNGGSTLCALQCLEKLYGDKWNSFTILLIHS

SEQ ID NO: 380 - protein, 266 aa, Artificial Sequence; identical to SEQ ID NO: 345
  Synthetic peptide

SEQ ID NO: 381 - protein, 35 aa, Artificial Sequence; identical to SEQ ID NO: 356
  Synthetic peptide

SEQ ID NO: 382 - protein, 152 aa, Artificial Sequence; identical to SEQ ID NO: 358
  Synthetic peptide

SEQ ID NO: 383 - protein, 114 aa, Artificial Sequence; identical to SEQ ID NO: 379
  Synthetic peptide

SEQ ID NO: 384 - protein, 6 aa, Artificial Sequence
  Synthetic peptide
RYFFPK

SEQ ID NO: 385 - protein, 114 aa, Artificial Sequence; identical to SEQ ID NO: 379
  Synthetic peptide

SEQ ID NO: 386 - protein, 34 aa, Artificial Sequence; identical to SEQ ID NO: 372
  Synthetic peptide

SEQ ID NO: 387 - protein, 114 aa, Artificial Sequence; identical to SEQ ID NO: 379
  Synthetic peptide

SEQ ID NO: 388 - protein, 42 aa, Artificial Sequence
  Synthetic peptide
MPRKDRNSSRAESAQCQVLSCVIHGILLMAREIAVVVLPLSQ

SEQ ID NO: 389 - protein, 50 aa, Artificial Sequence
  Synthetic peptide
VGESGPTTNLRKGLLGPDPQGMDPSLPPGSTPYPCINMIGYFPLSGPHFT

SEQ ID NO: 390 - protein, 33 aa, Artificial Sequence
  Synthetic peptide
VRLPSPFNTHPQHLLWAFTHDPEPSTSEAVAGR

SEQ ID NO: 391 - protein, 38 aa, Artificial Sequence
  Synthetic peptide
DQPFPAPWEPPYPPSLCSHSPAVSCSDPPHPPGSSSFS

SEQ ID NO: 392 - protein, 23 aa, Artificial Sequence
  Synthetic peptide
VSPPPPTFPRAGGCSHLKAPIPQ

SEQ ID NO: 393 - protein, 6 aa, Artificial Sequence
  Synthetic peptide
WSRCSP

SEQ ID NO: 394 - protein, 2 aa, Artificial Sequence
  Synthetic peptide
AS

SEQ ID NO: 395 - protein, 764 aa, Homo sapiens; identical to SEQ ID NO: 131

SEQ ID NO: 396 - protein, 936 aa, Homo sapiens; identical to SEQ ID NO: 209

SEQ ID NO: 397 - protein, 931 aa, Homo sapiens; identical to SEQ ID NO: 267

SEQ ID NO: 398 - protein, 175 aa, Homo sapiens; identical to SEQ ID NO: 302